(19) United States
(12) Patent Application Publication
(10) Pub. No.: US 2005/0197338 A1
(43) Pub. Date: Sep. 8, 2005

(54) Substituted pyrazole urea compounds for the treatment of inflammation

(76) Inventors: He Huang, Durham, NC (US); Michael A. Stealey, Libertyville, IL (US); Gunnar J. Hanson, Durham, NC (US); Patrick J. Lennon, Webster Groves, MO (US); Bruce C. Hamper, Kirkwood, MO (US); Jin Xie, Ballwin, MO (US); David S. Oburn, Ferguson, MO (US); Serge G. Wolfson, Chestefield, MO (US); Theresa R. Fletcher, Kirkwood, MO (US); Richard F. Heier, Columbia, IL (US); Matthew T. Reding, University City, MO (US); Michael T. Clare, Skokie, IL (US)

Correspondence Address: PHARMACIA CORPORATION;GLOBAL PATENT DEPARTMENT, POST OFFICE BOX 1027, ST. LOUIS, MO, 63006, US

(21) Appl. No.: 10/970,769

(22) Filed: Oct. 21, 2004

Related U.S. Application Data
(60) Provisional application No. 60/512,868, filed on Oct. 21, 2003.

(51) Int. Cl. A61K 31/53; A61K 31/506; A61K 31/497; A61K 31/4709; A61K 31/454; A61K 31/427; A61K 31/422; A61K 31/416

(52) U.S. Cl. 514/241; 514/406; 514/252.05; 514/255.05; 514/256; 514/326; 514/365; 514/374; 514/397; 514/314

(57) ABSTRACT

Compounds of Formula I: 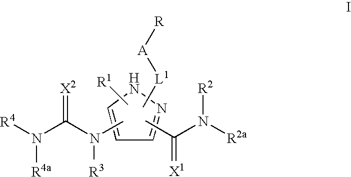
wherein X¹, X², L¹, A, R, L², R¹, R¹², R^{12a}, R¹³, R¹⁴ and R^{14a} are as defined herein, are disclosed.

### CROSS-REFERENCE TO RELATED APPLICATION

This application claims the benefit of U.S. Provisional Application Ser. No. 60/512,868, filed Oct. 21, 2003, the contents of which are hereby incorporated by reference in their entirety.

### FIELD OF THE INVENTION

This invention generally relates to anti-inflammatory pharmaceutical agents and specifically relates to pyrazole urea compounds as inhibitors of IKK-2, an IκB kinase. The invention is further related to compositions comprising such compounds, and methods for treating cancer, inflammation, and inflammation-associated disorders such as arthritis.

### BACKGROUND OF THE INVENTION

Rheumatoid arthritis is a common inflammatory disease affecting approximately 1% of the population. The disease is characterized by multiple painful swollen joints that severely limit the patient's daily function, and can progress to the destruction of the affected joints. A common treatment for rheumatoid arthritis is anti-inflammatory steroids. Steroids are clinically very effective, but are limited in their use because of multiple severe side-effects. Thus, a need exists for an anti-rheumatoid arthritis treatment that offers the potency of steroids without the associated toxicity. One of the mechanisms by which steroids exert their broad spectrum anti-inflammatory action is by inhibiting the activation of the transcription factor NF-κB. NF-κB plays a prominent role in immune and inflammatory responses by regulating the transcription of many early, inducible genes in a variety of cells including inflammatory enzymes such as COX-2 and iNOS. NF-κB is sequestered in an inactive form in the cytoplasm by a member of the IκB family of inhibitory proteins, and this prevents gene transcription of these responsive genes in the nucleus. Stimulation of cells leads to the phosphorylation, ubiquination and degradation of IκB thereby releasing NF-κB to the nucleus for activation of gene transcription. Chronic activation of NF-κB has been demonstrated in vascular endothelium and synovial lining cells from patients with RA. Recently the IκB kinases (IKK-1 and IKK-2), which phosphorylate IκB and thereby initiate its degradation, have been cloned and initially characterized; these kinases appear to represent the critical, common denominator in the activation of NF-κB since antisense or dominant-negative IKK constructs block NF-κB nuclear translocation and inhibit NF-κB linked reported genes. Therefore, IKK-1 and/or IKK-2 represent novel and powerful targets for drug development.

It has been reported that selective IKK-2 inhibitors could be useful for the treatment of inflammatory diseases. See, e.g., Karin et al., Nat. Revs. 3, 17-26, 2004.

U.S. Pat. No. 5,134,142 describes 1,5-diaryl pyrazoles, and specifically, 1-(4-fluorophenyl)-5-[4-(methylsulfonyl)phenyl]-3-trifluoromethyl pyrazole, as having anti-inflammatory activity.

PCT Patent Application No. WO 95/15316 discloses substituted pyrazolyl benzenesulfonamide derivatives having anti-inflammatory properties.

PCT Patent Application No. WO 01/58890 discloses pyrazole carboxamide derivatives, and other heteroaromatic carboxamide derivatives, and their use as IKK-2 inhibitors.

### SUMMARY OF THE INVENTION

This invention provides for, in part, IKK-2-inhibiting compounds of Formula I: 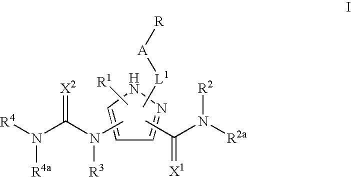
wherein X¹ is selected from the group consisting of O, S, and NR^{6a};
wherein X² is selected from the group consisting of O, S, and NR^{6b};
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b}.
wherein R is selected from the group consisting of hydrido and -L²R⁵;
wherein L¹ and L² are independently selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, alkylene, alkenylene, and alkynylene, wherein said R⁷, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR^{8a}, —(CH₂)ₑC(═O)R^{a}, —(CH₂)ₑC(═O)OR^{8a}, —(CH₂)ₑC(═O)NR^{8a}R^{8b}, —NR^{8a}R^{8b}, (CH₂)ₑOR^{8a}, —(CH₂)ₑNR^{8a}R^{8b}, and —(CH₂)ₑS(═O)_{f}R¹¹, or R¹ together with R^{4a} and the atoms to which they are attached form a heterocyclic ring moiety having the structure: 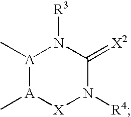
wherein R², R^{2a}, and R³ are independently selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R^{4a} is selected from the group consisting of hydrido, hydroxyl, alkoxy, alkyl, haloalkyl, aryl, and heteroaryl, or R^{4a} together with R¹ and the atoms to which they are attached form a heterocyclic ring having the structure: 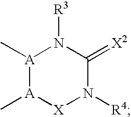
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl, and wherein R⁴, when R¹ together with R^{4a} and the atoms to which they are attached form a heterocyclic ring having 6 or more members, may form a double bond between the nitrogen to which R⁴ is attached and X;
wherein -A-A- represents any two adjacent atoms of the pyrazole ring in Formula I;
wherein X is selected from the group consisting of a bond, —(CHR^{9a})ₓ(CHR^{9b})_{y}(CHR^{9c})_{z}—, —NR^{9a}—, —CR^{9a}═CR^{9b}—(CHR^{9c})_{g}—, —(CH₂)ₕO—, —(C₂)ₕS—, —NR^{9a}—NR^{9b}, —N═N—, —O—NR^{9a}—, —S—NR^{9a}—, —CH═CH—CH₂—, —CH═CH—NR^{9a}—, —CH₂—O—CH₂—, —CH₂—S—CH₂—, —CH₂—N═N—, —NR^{9a}O—CH₂—, —CH₂—NR^{9a}—S—, and —NR^{9a}—S—CH₂—;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a R}^{14b}, —NR^{13a}C(═O)NR^{14a}R^{14b}, —C(═O)R^{13a}, and C(═O)NR^{12a}R^{12b};
wherein R^{6a}, R^{6b}, R⁷, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, O, N, and NR^{13a};
wherein a, d, and e are independently selected from the group consisting of 0, 1, 2, 3, 4, 5, 6, 7, or 8;
wherein b, c, f, and h are independently selected from the group consising of 0,1, or 2;
wherein g is 0 or 1;
wherein x, y, and z are independently 0, 1, 2, or 3, and wherein x+y+z<4;
or a pharmaceutically-acceptable salt thereof.

The instant invention is also directed to pharmaceutical compositions comprising a compound of Formula I or a pharmaceutically-acceptable salt thereof, as defined above, and a pharmaceutically acceptable carrier, diluent, or adjuvant.

The instant invention is also directed to a method of treating or preventing inflammation or an inflammation-associated disorder, the method comprising administering a compound of Formula I or a pharmaceutically acceptable salt thereof to a subject in need of such treatment or susceptible to such inflammation or inflammation-associated disorder.

Other objects of the invention will be in part apparent and in part pointed out hereinafter.

### DETAILED DESCRIPTION OF THE PREFERRED EMBODIMENTS

In accordance with the present invention, Applicants have discovered a class of IKK-2-inhibiting compounds of Formula I: 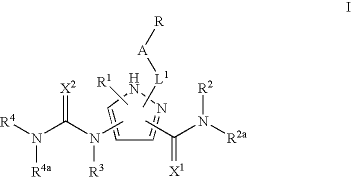
wherein X¹ is selected from the group consisting of O, S, and NR^{6a};
wherein X² is selected from the group consisting of O, S, and NR^{6b};
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}═O)NR^{14a}R^{14b}, —C(═O)R^{13a}, and C(═O)NR^{12a}R^{12b};
wherein R is selected from the group consisting of hydrido and -L²R⁵;
wherein L¹ and L² are independently selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, alkylene, alkenylene, and alkynylene, wherein said R⁷, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, (CH₂)ₑOR^{8a}, —(CH₂)ₑNR^{8a}R^{8b}, and —(CH₂)ₑS(═O)_{f}R¹¹, or R¹ together with R^{4a} and the atoms to which they are attached form a heterocyclic ring moiety having the structure: 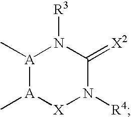
wherein R², R^{2a}, and R³ are independently selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R^{4a} is selected from the group consisting of hydrido, hydroxyl, alkoxy, alkyl, haloalkyl, aryl, and heteroaryl, or R^{4a} together with R¹ and the atoms to which they are attached form a heterocyclic ring having the structure: 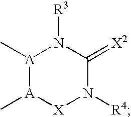
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl, and wherein R⁴, when R¹ together with R^{4a} and the atoms to which they are attached form a heterocyclic ring having 6 or more members, may form a double bond between the nitrogen to which R⁴ is attached and X;
wherein -A-A- represents any two adjacent atoms of the pyrazole ring in Formula I;
wherein X is selected from the group consisting of a bond, —(CHR^{9a}), (CHR^{9b}), (CHR^{9c})_{z}—, —NR^{9a}—, —CR^{9a}═CR^{9b}—(CHR^{9c})_{g}—, —(CH₂)ₕO—, —(CH₂)ₙS—, —NR^{9a}—NR^{9b}, —N═N—, —O—NR^{9a}, —S—NR^{9a}—, —CH═CH—CH₂—, —CH═CH—NR^{9a}, —CH₂—O—CH₂—, —CH₂—S—CH₂—, —CH₂—N═N—, —NR^{9a}O—CH₂—, —CH₂—NR^{9a}—S_, and —NR^{9a}—S—CH₂—;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R^{6a}, R^{6b}, R⁷, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂O, N, and NR^{13a};
wherein a, d, and e are independently selected from the group consisting of 0, 1, 2, 3, 4, 5, 6, 7, or 8;
wherein b, c, f, and h are independently selected from the group consising of 0, 1, or 2;
wherein g is 0 or 1;
wherein x, y, and z are independently 0,1, 2, or 3, and wherein x+y+z<4;
or a pharmaceutically-acceptable salt thereof.

Compounds of Formula I may be useful for treating, among other things, inflammation in a subject, such as, as an analgesic in the treatment of pain and headaches, or as an antipyretic for the treatment of fever. For example, compounds of the present invention may be useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis.

Compounds of the invention may be further useful in the treatment of frailty, asthma, chronic obstructive pulmonary disease (COPD), bronchitis, menstrual cramps (e.g., dysmenorrhea), premature labor, tendinitis, bursitis, dermatological conditions such as psoriasis, eczema, burns, sunburn, dermatitis, pancreatitis, hepatitis, and from post-operative inflammation including from ophthalmic surgery such as cataract surgery and refractive surgery. Compounds of the invention also would be useful to treat gastrointestinal conditions such as inflammatory bowel disease, Crohn's disease, gastritis, irritable bowel syndrome and ulcerative colitis. Compounds of the invention would be useful for the prevention or treatment of cancer, such as colorectal cancer, and cancer of the breast, lung, prostate, bladder, cervix and skin, as well as treatment of cancer stem cells. Compounds of the invention would be useful in treating inflammation and tissue damage in such diseases as vascular diseases, migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like.

The compounds would also be useful in the treatment of pulmonary inflammation, such as that associated with viral infections and cystic fibrosis. The compounds would also be useful for the treatment of certain central nervous system disorders, such as cortical dementias including Alzheimer's disease, and central nervous system damage resulting from stroke, ischemia and trauma. The compounds of the invention are useful as anti-inflammatory agents, such as for the treatment of arthritis, with the additional benefit of having significantly less harmful side effects. These compounds would also be useful in the treatment of allergic rhinitis, respiratory distress syndrome, and atherosclerosis. The compounds would also be useful in the treatment of pain, but not limited to postoperative pain, dental pain, muscular pain, and pain resulting from cancer. The compounds would be useful for the prevention of dementias, such as Alzheimer's disease.

Besides being useful for human treatment, these compounds are also useful for veterinary treatment of companion animals, exotic animals and farm animals, including mammals, rodents, and the like. More preferred animals include horses, dogs, and cats.

The present compounds may also be used in co-therapies, partially or completely, in place of other conventional antiinflammatory therapies, such as together with steroids, NSAIDs, COX-2 selective inhibitors, 5-lipoxygenase inhibitors, LTB₄ antagonists and LTA₄ hydrolase inhibitors.

Other conditions in which the compounds of the present invention may provide an advantage include cardiovascular ischemia, diabetes (type I or type II), congestive heart failure, myocarditis, atherosclerosis, migraine, glaucoma, aortic aneurysm, reflux esophagitis, diarrhea, irritable bowel syndrome, cystic fibrosis, emphysema, asthma, bronchiectasis, hyperalgesia (allodynia), and cerebral ischemia (both focal ischemia, thrombotic stroke and global ischemia (for example, secondary to cardiac arrest).

The compounds of the present invention may also be useful in the treatment of pain including somatogenic (either nociceptive or neuropathic), both acute and chronic. A compound of the present invention could be used in any situation including neuropathic pain that a common NSAID or opioid analgesic would traditionally be administered.

Conjunctive treatment of a compound of the present invention with an antineoplastic agent may produce a beneficial effect or alternatively reduce the toxic side effects associated with chemotherapy by reducing the therapeutic dose of the side effect-causing agent needed for therapeutic efficacy or by directly reducing symptoms of toxic side effects caused by the side effect-causing agent. A compound of the present invention may further be useful as an adjunct to radiation therapy to reduce side effects or enhance efficacy. In the present invention, another agent which can be combined therapeutically with a compound of the present invention includes any therapeutic agent which is capable of inhibiting the enzyme cyclooxygenase-2 (“COX-2”). Preferably such COX-2 inhibiting agents inhibit COX-2 selectively relative to the enzyme cyclooxygenase-1 (“COX-1”). Such a COX-2 inhibitor is known as a “COX-2 selective inhibitor”. More preferably, a compound of the present invention can be therapeutically combined with a COX-2 selective inhibitor wherein the COX-2 selective inhibitor selectively inhibits COX-2 at a ratio of at least 10:1 relative to inhibition of COX-1, more preferably at least 30:1, and still more preferably at least 50:1 in an in vitro test. COX-2 selective inhibitors useful in therapeutic combination with the compounds of the present invention include celecoxib, valdecoxib, deracoxib, etoricoxib, rofecoxib, ABT-963 (2-(3,4-difluorophenyl)-4-(3-hydroxy-3-methyl-1-butoxy)-5-[4-(methylsulfonyl)phenyl-3(2H)-pyridazinone; described in PCT Publication No. WO 00/24719), or meloxicam. A compound of the present invention can also be advantageously used in therapeutic combination with a prodrug of a COX-2 selective inhibitor, for example parecoxib.

Another chemotherapeutic agent which may be useful in combination with a compound of the present invention can be selected, for example, from the following non-comprehensive and non-limiting list: o-difluoromethylornithine (DFMO), 5-FU-fibrinogen, acanthifolic acid, aminothiadiazole, brequinar sodium, carmofur, Ciba-Geigy CGP-30694, cyclopentyl cytosine, cytarabine phosphate stearate, cytarabine conjugates, Lilly DATHF, Merrill Dow DDFC, dezaguanine, dideoxycytidine, dideoxyguanosine, didox, Yoshitomi DMDC, doxifluridine, Wellcome EHNA, Merck & Co. EX-015, fazarabine, floxuridine, fludarabine phosphate, 5-fluorouracil, N-(2′-furanidyl)-5-fluorouracil, Daiichi Seiyaku FO-152, isopropyl pyrrolizine, Lilly LY-188011, Lilly LY-264618, methobenzaprim, methotrexate, Wellcome MZPES, norspermidine, NCI NSC-127716, NCI NSC-264880, NCI NSC-39661, NCI NSC-612567, Warner-Lambert PALA, pentostatin, piritrexim, plicamycin, Asahi Chemical PL-AC, Takeda TAC-788, thioguanine, tiazofurin, Erbamont TIF, trimetrexate, tyrosine kinase inhibitors, tyrosine protein kinase inhibitors, Taiho UFT, uricytin, Shionogi 254-S, aldo-phosphamide analogues, altretamine, anaxirone, Boehringer Mannheim BBR-2207, bestrabucil, budotitane, Wakunaga CA-102, carboplatin, carmustine, Chinoin-139, Chinoin-153, chlorambucil, cisplatin, cyclophosphamide, American Cyanamid CL-286558, Sanofi CY-233, cyplatate, Degussa D-19-384, Sumimoto DACHP(Myr)₂, diphenylspiromustine, diplatinum cytostatic, Erba distamycin derivatives, Chugai DWA-2114R, ITI E09, elmustine, Erbamont FCE-24517, estramustine phosphate sodium, fotemustine, Unimed G-6-M, Chinoin GYKI-17230, hepsul-fam, ifosfamide, iproplatin, lomustine, mafosfamide, mitolactol, Nippon Kayaku NK-121, NCI NSC-264395, NCI NSC-342215, oxaliplatin, Upjohn PCNU, prednimustine, Proter PTT-119, ranimustine, semustine, SmithKline SK&F-101772, Yakult Honsha SN-22, spiromus-tine, Tanabe Seiyaku TA-077, tauromustine, temozolomide, teroxirone, tetraplatin, trimelamol, Taiho 4181-A, aclarubicin, actinomycin D, actinoplanone, Erbamont ADR-456, aeroplysinin derivative, Ajinomoto AN-201-II, Ajinomoto AN-3, Nippon Soda anisomycins, anthracycline, azino-mycin-A, bisucaberin, Bristol-Myers BL-6859, Bristol-Myers BMY-25067, Bristol-Myers BMY-25551, Bristol-Myers BMY-26605, Bristol-Myers BMY-27557, Bristol-Myers BMY-28438, bleomycin sulfate, bryostatin-1, Taiho C-1027, calichemycin, chromoximycin, dactinomycin, daunorubicin, Kyowa Hakko DC-102, Kyowa Hakko DC-79, Kyowa Hakko DC-88A, Kyowa Hakko DC89-A1, Kyowa Hakko DC92-B, ditrisarubicin B, Shionogi DOB-41, doxorubicin, doxorubicin-fibrinogen, elsamicin-A, epirubicin, erbstatin, esorubicin, esperamicin-Al, esperamicin-Alb, Erbamont FCE-21954, Fujisawa FK-973, fostriecin, Fujisawa FR-900482, glidobactin, gregatin-A, grincamycin, herbimycin, idarubicin, illudins, kazusamycin, kesarirhodins, Kyowa Hakko KM-5539, Kirin Brewery KRN-8602, Kyowa Hakko KT-5432, Kyowa Hakko KT-5594, Kyowa Hakko KT-6149, American Cyanamid LL-D49194, Meiji Seika ME 2303, menogaril, mitomycin, mitoxantrone, SmithKline M-TAG, neoenactin, Nippon Kayaku NK-313, Nippon Kayaku NKT-01, SRI International NSC-357704, oxalysine, oxaunomycin, peplomycin, pilatin, pirarubicin, porothramycin, pyrindamycin A, Tobishi R-I, rapamycin, rhizoxin, rodorubicin, sibanomicin, siwenmycin, Sumitomo SM-5887, Snow Brand SN-706, Snow Brand SN-07, sorangicin-A, sparsomycin, SS Pharmaceutical SS-21020, SS Pharmaceutical SS-7313B, SS Pharmaceutical SS-9816B, steffimycin B, Taiho 4181-2, talisomycin, Takeda TAN-868A, terpentecin, thrazine, tricrozarin A, Upjohn U-73975, Kyowa Hakko UCN-10028A, Fujisawa WF-3405, Yoshitomi Y-25024 zorubicin, α-carotene, α-difluoromethyl-arginine, acitretin, Biotec AD-5, Kyorin AHC-52, alstonine, amonafide, amphethinile, amsacrine, Angiostat, ankinomycin, anti-neoplaston A10, antineoplaston A2, antineoplaston A3, antineoplaston A5, antineoplaston AS2-1, Henkel APD, aphidicolin glycinate, asparaginase, Avarol, baccharin, batracylin, benfluron, benzotript, Ipsen-Beaufour BIM-23015, bisantrene, Bristo-Myers BMY-40481, Vestar boron-10, bromofosfamide, Wellcome BW-502, Wellcome BW-773, caracemide, carmethizole hydrochloride, Ajinomoto CDAF, chlorsulfaquinoxalone, Chemex CHX-2053, Chemex CHX-100, Warner-Lambert CI-921, Warner-Lambert CI-937, Warner-Lambert CI-941, Warner-Lambert CI-958, clanfenur, claviridenonei, ICN compound 1259, ICN compound 4711, Contracan, Yakult Honsha CPT-11, crisnatol, curaderm, cytochalasin B, cytarabine, cytocytin, Merz D-609, DABIS maleate, dacarbazine, datelliptinium, didemnin-B, dihaematoporphyrin ether, dihydrolenperone, dinaline, distamycin, Toyo Pharmar DM-341, Toyo Pharmar DM-75, Daiichi Seiyaku DN-9693, elliprabin, elliptinium acetate, Tsumura EPMTC, ergotamine, etoposide, etretinate, fenretinide, Fujisawa FR-57704, gallium nitrate, genkwadaphnin, Chugai GLA-43, Glaxo GR-63178, grifolan NMF-5N, hexadecylphosphocholine, Green Cross HO-221, homoharringtonine, hydroxyurea, BTG ICRF-187, ilmofosine, isoglutamine, isotretinoin, Otsuka JI-36, Ramot K-477, Otsuak K-76COONa, Kureha Chemical K-AM, MECT Corp KI-8110, American Cyanamid L-623, leukoregulin, lonidamine, Lundbeck LU-23-112, Lilly LY-186641, NCI (US) MAP, marycin, Merrel Dow MDL-27048, Medco MEDR-340, merbarone, merocyanine derivatives, methylanilinoacridine, Molecular Genetics MGI-136, minactivin, mitonafide, mitoquidone, mopidamol, motretinide, Zenyaku Kogyo MST-16, N-(retinoyl)amino acids, Nisshin Flour Milling N-021, N-acylated-dehydroalanines, nafazatrom, Taisho NCU-190, nocodazole derivative, Normosang, NCI NSC-145813, NCI NSC-361456, NCI NSC-604782, NCI NSC-95580, octreotide, Ono ONO-112, oquizanocine, Akzo Org-10172, pancratistatin, pazelliptine, Warner-Lambert PD-i 11707, Warner-Lambert PD-115934, Warner-Lambert PD-131141, Pierre Fabre PE-1001, ICRT peptide D, piroxantrone, polyhaematoporphyrin, polypreic acid, Efamol porphyrin, probimane, procarbazine, proglumide, Invitron protease nexin 1, Tobishi RA-700, razoxane, Sapporo Breweries RBS, restrictin-β, retelliptine, retinoic acid, Rhone-Poulenc RP-49532, Rhone-Poulenc RP-56976, SmithKline SK&F-104864, Sumitomo SM-108, Kuraray SMANCS, SeaPharm SP-10094, spatol, spirocyclopropane derivatives, spirogermanium, Unimed, SS Pharmaceutical SS-554, strypoldinone, Stypoldione, Suntory SUN 0237, Suntory SUN 2071, superoxide dismutase, Toyama T-506, Toyama T-680, taxol, Teijin TEI-0303, teniposide, thaliblastine, Eastman Kodak TJB-29, tocotrienol, Topostin, Teijin TT-82, Kyowa Hakko UCN-01, Kyowa Hakko UCN-1028, ukrain, Eastman Kodak USB6, vinblastine sulfate, vincristine, vindesine, vinestramide, vinorelbine, vintriptol, vinzolidine, withanolides, Yamanouchi YM-534, uroguanylin, combretastatin, dolastatin, idarubicin, epirubicin, estramustine, cyclophosphamide, 9-amino-2-(S)-camptothecin, topotecan, irinotecan (Camptosar), exemestane, decapeptyl (tryptorelin), or an omega-3 fatty acid.

Examples of radioprotective agents which may be used in a combination therapy with the compounds of this invention include AD-5, adchnon, amifostine analogues, detox, dimesna, I-102, MM-159, N-acylated-dehydroalanines, TGF-Genentech, tiprotimod, amifostine, WR-151327, FUT-187, ketoprofen transdermal, nabumetone, superoxide dismutase (Chiron) and superoxide dismutase Enzon.

The compounds of the present invention may also be useful in treatment or prevention of angiogenesis-related disorders or conditions, for example, tumor growth, metastasis, macular degeneration, and atherosclerosis.

In a further embodiment, the present invention also provides therapeutic combinations for the treatment or prevention of ophthalmic disorders or conditions such as glaucoma. For example the present inventive compounds advantageously may be used in therapeutic combination with a drug which reduces the intraocular pressure of patients afflicted with glaucoma. Such intraocular pressure-reducing drugs include without limitation latanoprost, travoprost, bimatoprost, or unoprostol. The therapeutic combination of a compound of the present invention plus an intraocular pressure-reducing drug may be useful because each is believed to achieve its effects by affecting a different mechanism.

In another combination of the present invention, the present inventive compounds can be used in therapeutic combination with an antihyperlipidemic or cholesterol-lowering drug such as a benzothiepine or a benzothiazepine antihyperlipidemic drug. Examples of benzothiepine antihyperlipidemic drugs useful in the present inventive therapeutic combination can be found in U.S. Pat. No. 5,994,391, herein incorporated by reference. Some benzothiazepine antihyperlipidemic drugs are described in PCT Publication No. WO 93/16055. Alternatively, the antihyperlipidemic or cholesterol-lowering drug useful in combination with a compound of the present invention can be an HMG Co-A reductase inhibitor. Examples of HMG Co-A reductase inhibitors useful in the present therapeutic combination include, individually, benfluorex, fluvastatin, lovastatin, pravastatin, simvastatin, atorvastatin, cerivastatin, bervastatin, ZD-9720 (described in PCT Publication No. WO 97/06802), ZD-4522 (CAS No. 147098-20-2 for the calcium salt; CAS No. 147098-18-8 for the sodium salt; described in European Patent No. EP 521471), BMS 180431 (CAS No. 129829-03-4), or NK-104 (CAS No. 141750-63-2). The therapeutic combination of a compound of the present invention plus an antihyperlipidemic or cholesterol-lowering drug may be useful, for example, in reducing the risk of formation of atherosclerotic lesions in blood vessels. For example, atherosclerotic lesions often initiate at inflamed sites in blood vessels. It is established that antihyperlipidemic or cholesterol-lowering drug reduce risk of formation of atherosclerotic lesions by lowering lipid levels in blood. Without limiting the invention to a single mechanism of action, it is believed that one way the compounds of the present combination may work in concert to provide improved control of atherosclerotic lesions by, for example, reducing inflammation of the blood vessels in concert with lowering blood lipid levels.

In another embodiment of the invention, the present compounds can be used in combination with other compounds or therapies for the treatment of central nervous conditions or disorders such as migraine. For example, the present compounds can be used in therapeutic combination with caffeine, a 5-HT-1B/1D agonist (for example, a triptan such as sumatriptan, naratriptan, zolmitriptan, rizatriptan, almotriptan, or frovatriptan), a dopamine D4 antagonist (e.g., sonepiprazole), aspirin, acetaminophen, ibuprofen, indomethacin, naproxen sodium, isometheptene, dichloralphenazone, butalbital, an ergot alkaloid (e.g., ergotamine, dihydroergotamine, bromocriptine, ergonovine, or methyl ergonovine), a tricyclic antidepressant (e.g., amitriptyline or nortriptyline), a serotonergic antagonist (e.g., methysergide or cyproheptadine), a β-andrenergic antagonist (e.g., propranolol, timolol, atenolol, nadolol, or metprolol), or a monoamine oxidase inhibitor (e.g., phenylzine or isocarboxazid).

The present invention includes compounds that selectively inhibit IKK-2 over other kinases. Such other kinases include, but are not limited to, Abl(h), Abl(T3151), Abl(T3151), AMPK, Aurora-A, BTK, CaMKII, CaMKIV, CDK1/cyclinB, CDK2, CDK2/cyclin A, CDK2/cyclinE, CHK1, CHK2, CK1, CK1(y), CK1δ, CK2, c-RAF(h), CSK, cSRC(h), DYRK1a, ERK2, Fyn, GSK3β, IGF-1R, IKK1, IKKi, IKK2(h), JNK/SAPK1c, JNK1, JNK1α1(h), JNK2, JNK2α2(h), JNK3, Lck, MAPK1(h), MAPK2(h), MAPK2/ERK2, MAPKAP-K1a, MAPKAP-K2, MEK1, MK-2, MK-3, MKK1, MKK4, MKK6, MKK7, MKK7β(h), MNK, MRSK2/APKAPk1b, MSK, MSK1, NEK2a, NEK6, p38α, p38β, p38δ, p38γ, p70 S6K, PAK2, PDGFRβ, PDK1, PHK, PKA, PKBΔph, PKCζ, PKCα, PKCγ, PKCδ, PKCε, PRAK, ROCK-II, Rskl, Rsk2, RSKB, SAPK2a/p38, SAPK2b, SAPK2b/p38β2, SAPK3, SAPK3/p38g, SAPK4, SAPK4/p38d, SGK, TBK-1, and ZAP-70. The compounds may have an IKK-2 IC₅₀ of less than about 10 μM, preferably less than about 1 μM, and have a selectivity ratio of IKK-2 inhibition over IKK-1 inhibition of at least 50, or at least 100. The compounds may have an IKK-1 IC₅₀ of greater than 10 μM, or greater than 100 μM.

In one preferred embodiment, the compound of Formula I is a compound wherein A is selected from the group consisting of C₃₋₁₂ cycloalkyl, C₃₋₁₂ cycloalkenyl, 3- to 12-membered heterocycloalkyl, 3- to 12-membered heterocycloalkenyl, C₁₋₂ aryl, and 5- to 12-membered heteroaryl, wherein A may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈, alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a} N(═CR^{13a}R^{13b}), —(CH₂)ₐOR¹¹, —(CH₂)ₐNR^{12a}R^{12b}, (CH₂)ₐS(═O)_{b}R, —(CH₂)ₐCO₂R¹¹, S—SO₂NR^{12a}R^{12b}, —NR^{13a}R^{13b}, —NR^{12a}C(═O)R^{13a}, NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ and L² are independently selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, C₁₋₆ alkylene, C₂₋₆ alkenylene, and C₂₋₆ alkynylene, wherein said R⁷, alkylene, alkenylene, and alkynylene moieties may be substituted by one or more substituents independently selected from R¹⁵;
wherein R¹ is selected from the group consisting of hydrido, cyano, C₁₋₆ alkyl, C₂₋₆ alkenyl, halo, C₁₋₆ haloalkyl, C₁₋₆ hydroxyalkyl, cyano(C₁₋₆ alkyl), C₅₋₁₂ aryl, 5- to 12-membered heteroaryl, —ORaa, —(CH₂)ₑC(═O)R^{a}, —(CH₂)ₑC(═O)OR^{a}, —(CH₂)ₑC(═O)NR^{8a}R^{8b}, —NR^{8a}R^{8b}, —(CH₂)ₑR^{8a}, (CH₂)ₑNR^{8a}R^{8b}, and (CH₂)ₑS(═O)_{f}R¹¹, or R¹ together with R^{4a} and the atoms to which they are attached form a 5- to 8-membered heterocyclic ring moiety having the structure: 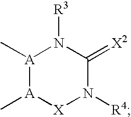
wherein R², R^{2a}, and R³ are independently selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R^{4a} is selected from the group consisting of hydrido, hydroxyl, C₁₋₆ alkoxy, C₁₋₆ alkyl, C₁₋₆ haloalkyl, C₅₋₁₂ aryl, and 5- to 12-membered heteroaryl, or R^{4a} together with R¹ and the atoms to which they are attached form a 5- to 8-membered heterocyclic ring having the structure: 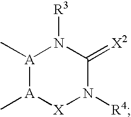
wherein R⁴ is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl, and wherein R⁴, when R¹ together with R^{4a} the atoms to which they are attached form a heterocyclic ring having 6 or more members, may form a double bond between the nitrogen to which R⁴ is attached and X;
wherein R⁵ is selected from the group consisting of C₁₋₆ alkyl, C₃₋₁₂ cycloalkyl, C₃₋₁₂ cycloalkenyl, 3- to 12-membered heterocycloalkyl, 3- to 12-membered heterocycloalkenyl, 5- to 12-membered heteroaryl, and C₅₋₁₂ aryl, wherein R⁵ may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═)NR^{14a}R^{14b}, —C(═O)R^{13a}, and —C(═O)NR^{12a}R^{12b};
wherein R^{6a}, R^{6b}, R⁷, R^{8a}, R^{8b}, R^{9a}, R^{9b}, R^{9c}, and R¹⁵ are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₄ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R¹⁰ is selected from the group consisting of C₃₋₁₂ cycloalkyl, C₃₋₁₂ cycloalkenyl, C₅₋₁₂ aryl, 3- to 12-membered heterocycloalkenyl, 5- to 12-membered heteroaryl, and C₂₋₆ alkenyl, wherein R¹⁰ is optionally substituted with one or more substituents selected from the group consisting of R^{13a};
wherein R¹¹, R^{12a}, and R^{12b} are independently selected from the group consisting of hydrido, C₅₋₁₂ aryl, 5- to 12-membered heteroaryl, 4- to 18-membered heteroaralkyl, C₁₋₆ alkyl, C₁₋₆ haloalkyl, C₂₋₆ alkenyl, C₂₋₆ alkynyl, C₁₋₆ hydroxyalkyl, C₁₋₆ aminoalkyl, C₂₋₁₂ alkylaminoalkyl, N-N-di(C₁₋₆ alkyl)amino(C₁₋₆alkyl), C₁₋₆ alkoxy, C₂₋₁₂ alkoxyalkyl, 3- to 12-membered heterocycloalkyl, 3- to 12-membered heterocycloalkenyl, C₃₋₁₂ cycloalkyl, C₄₋₁₈ cycloalkylalkyl, C₄₋₁₈ aralkyl, and C₄₋₁₈ aralkylamino, wherein said aryl is optionally substituted with one or more radicals selected from the group consisting of C₁₋₆ alkyl, C₁₋₆ aminoalkyl, C₁₋₆ alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₂₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{14a} and R^{14b} are independently selected from the group consisting of hydrido, C₁₋₆ alkyl, 5- to 12-membered heteroaryl, 3- to 12-membered heterocycloalkenyl, C₁₋₆ haloalkyl, C₄₋₁₈ aralkylamino, 4- to 18-membered heteroaralkyl, C₅₋₁₂ aryl, and C₄₋₁₈ aralkyl, wherein said aryl, heteroaryl, heterocycloalkenyl, and aralkyl moieties are optionally substituted with one or more substituents selected from the group consisting of C₁₋₆ alkyl, C₁₋₆ alkoxy, halo, C₁₋₆ haloalkyl, cyano, C₁₋₆ haloalkoxy, C₂₋₁₀ acyl, carboxyl, hydroxy, C₁₋₆ hydroxyalkoxy, phenoxy, benzyloxy, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl, wherein R^{14a} and R¹⁴bmay be taken together to form a 3- to 7-membered carbocyclic ring having 1 to 3 heteroatoms selected from S, SO, SO₂, O, N, and NR^{13a};
or a pharmaceutically-acceptable salt thereof.

In one particularly preferred embodiment, the compound of Formula 1 is a compound wherein A is selected from the group consisting of cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein A may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, —C(═N)NR^{12a}R^{12b}, —NR^{12a}N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ and L² are independently selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, methylene, ethylene, propylene, butylene, pentylene, hexylene, ethenylene, propenylene, butenylene, pentenylene, and ethynylene, propynylene, butynylene, pentynylene, wherein said R⁷, methylene, ethylene, propylene, butylene, pentylene, hexylene, ethenylene, propenylene, butenylene, pentenylene, and ethynylene, propynylene, butynylene, pentynylene moieties may be substituted by one or more substituents independently selected from R¹⁵;
wherein R¹ is selected from the group consisting of hydrido, cyano, methyl, ethyl, propyl, butyl, pentyl, hexyl, ethenyl, propenyl, butenyl, pentenyl, chloro, fluoro, bromo, iodo, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, cyanomethyl, cyanoethyl, cyanopropyl, cyanobutyl, cyanohexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, —OR^{8a}, —(CH₂)ₑC(═O)R^{8a}, —(CH₂)ₑC(═O)OR^{8a}, —(CH₂)ₑC(═O)NR^{a}R^{8b}, —NR^{8a}R^{8b}, —(CH₂)ₑOR^{8a}, —(CH₂)ₑNR^{8a}R^{8b}, and —(CH₂)ₑS(═O)_{f}R¹¹, or R¹ together with R^{4a} and the atoms to which they are attached form a 5- to 8-membered heterocyclic ring moiety having the structure: 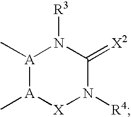
wherein R², R^{2a}, and R³ are independently selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{4a} is selected from the group consisting of hydrido, hydroxyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, or R^{4a} together with R¹ and the atoms to which they are attached form a 5- to 8-membered heterocyclic ring having the structure: 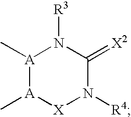
wherein R⁴ is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonyl butyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of methyl, ethyl, propyl, butyl, pentyl, hexyl, cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and phenyl, biphenyl, naphthyl, indenyl, wherein R⁵ may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), (CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR¹²R^{12b};
wherein R^{6a}, R^{6b}, R⁷, R⁸, R^{8b}, R^{9a}, R^{9b}, R^{9c}, and R⁵ are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R¹⁰ is selected from the group consisting of cyanomethyl, cyanoethyl, cyanopropyl, cyanobutyl, cyanopentyl, cyanohexyl, cyclopropenyl, cyclobutenyl, cyclopentenyl, cyclohexenyl, phenyl, biphenyl, naphthyl, indenyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, ethenyl, propenyl, butenyl, and pentenyl, wherein R¹⁰ is optionally substituted with one or more substituents selected from the group consisting of R^{13a};
wherein R¹¹, R^{12a}, and R^{12b} are independently selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, methyl, ethyl, propyl, butyl, pentyl, hexyl, chloromethyl, dichloromethyl, trichloromethyl, fluoromethyl, difluoromethyl, trifluoromethyl, ethenyl, propenyl, butenyl, pentenyl, ethynyl, propynyl, butynyl, pentynyl, hydroxymethyl, hydroxyethyl, hydroxypropyl, hydroxybutyl, hydroxypentyl, hydroxyhexyl, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, N,N-dimethylaminomethyl, N,N-dimethylaminoethyl, N-methyl-N-ethylaminomethyl, N-methyl-N-ethylaminoethyl, N-methyl-N-propylaminomethyl, N-methyl-N-propylaminoethyl, N,N-diethylaminomethyl, N,N-diethylaminoethyl, N-ethyl-N-propylaminomethyl, N-ethyl-N-propylaminoethyl, N,N-dipropylaminomethyl, N,N-dipropylaminoethyl, methoxy, ethoxy, propoxy, butoxy, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl, cyclohexylethyl, benzyl, phenylethyl, benzylamino, and phenylethylamino, wherein said phenyl, biphenyl, naphthyl, indenyl is optionally substituted with one or more radicals selected from the group consisting of methyl, ethyl, propyl, butyl, pentyl, hexyl, aminomethyl, aminoethyl, aminopropyl, aminobutyl, aminopentyl, aminohexyl, methoxy, ethoxy, propoxy, butoxy and chloro, fluoro, bromo, iodo, wherein R^{12a} and R^{12b} may be taken together to form a 3- to 7-membered carbocyclic ring having from 1 to 3 heteroatoms selected from S, SO, SO₂O, N, and NR^{13a};
wherein R^{13a} and R^{13b} are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{14a} and R^{14b} are independently selected from the group consisting of hydrido, methyl, ethyl, propyl, butyl, pentyl, hexyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, chloromethyl, dichloromethyl, trichloromethyl, fluoromethyl, difluoromethyl, trifluoromethyl, benzylamino, phenylethylamino, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, phenyl, biphenyl, naphthyl, indenyl, benzyl, and phenylethyl, wherein said phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, benzyl, and phenylethyl moieties are optionally substituted with one or more substituents selected from the group consisting of methyl, ethyl, propyl, butyl, pentyl, hexyl, methoxy, ethoxy, propoxy, butoxy, chloro, fluoro, bromo, iodo, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, benzyloxy, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

In a preferred embodiment, the compound of Formula I is a compound of Formula II: 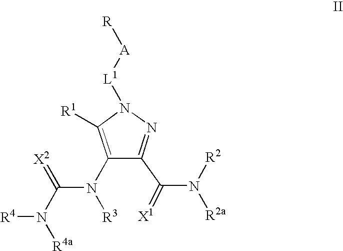
wherein X¹, X², L¹, L², A, R, R¹, R², R^{2a}, R³, R⁴, and R^{4a} are as defined above for Formula I.

In a preferred embodiment, the compound of Formula I is a compound of Formula III: 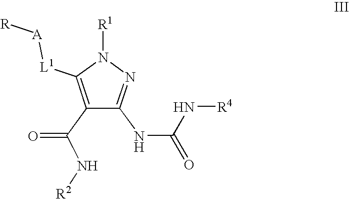
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR¹³SO₂NR^{14a}R^{14b}, —NR^{13a}C(═O)NR^{14a}R^{14b}, —C(═O)R^{13a}, and C(═O)NR^{12a}R^{12b};
wherein R is selected from the group consisting of hydrido and -L²R⁵;
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}, —R^{7a}C(═O)—, —OR^{7a}C(═O)—, and alkynylene, wherein said R^{7a}, alkylene, alkenylene, and alkynylene moieties may be substituted by one or more substituents independently selected from R⁵;
wherein L² is selected from the group consisting of a bond, —O—, —S(═O)ᵣ—, —O(CH₂)_{d}—, —C(═O)—, —OC(═O)—, —C(═O)O—, —OC(═O)R^{7b}, C(═O)R^{7b}, —R^{7b}C(═O)—, —OR^{7b}C(═O)—, alkylene, alkenylene, and alkynylene, wherein said R^{7b}, alkylene, alkenylene, and alkynylene moieties may be substituted by one or more substituents independently selected from R⁵;
wherein R¹ is selected from the group consisting of hydrido, cyano, alkyl, alkenyl, halo, haloalkyl, hydroxyalkyl, cyanoalkyl, aryl, heteroaryl, —OR⁸, (CH₂)ₑC(═O)R^{a}, —(CH₂)ₑC(═O)OR^{8a}, —(CH₂)ₑC(═O)NR^{8a}R^{8b}, —NR^{8a}R^{8b}, —(CH)ₑOR^{8a}, —(CH₂)ₑNR^{8a}R^{8b}, and —(CH₂)ₑS(═O)_{f}R¹¹;
wherein R² is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R^{7a} is selected from the group consisting of hydrido, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{7b}, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R¹³a and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, O, N, and NR^{13a};
wherein a, d, and e are independently selected from the group consisting of 0, 1, 2, 3, 4, 5, 6, 7, or 8;
wherein b, c, and f are independently selected from the group consising of 0, 1, or 2;
or a pharmaceutically-acceptable salt thereof.

In one preferred embodiment, the compound of Formula II is a compound wherein A is selected from the group consisting of C₃₋₁₂ cycloalkyl, C₃₋₁₂ cycloalkenyl, 3- to 12-membered heterocycloalkyl, 3- to 12-membered heterocycloalkenyl, C₅₋₁₂ aryl, and 5- to 12-membered heteroaryl, wherein A may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}, —R^{7a}C(═O)—, —OR^{7a}C(═O)—, and C₂₋₆ alkynylene, wherein said R⁷ and alkynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, C₁₋₆ alkylene, C₂₋₆ alkenylene, and C₂₋₆ alkynylene, wherein said R⁷, alkylene, alkenylene, and alkynylene moieties may be substituted by one or more substituents independently selected from R¹⁵;
wherein R¹ is selected from the group consisting of hydrido, cyano, C₁₋₆ alkyl, C₂₋₆ alkenyl, halo, C₁₋₆ haloalkyl, C₁₋₆ hydroxyalkyl, cyano(C₁₋₆ alkyl), C₅₋₁₂ aryl, 5- to 12-membered heteroaryl, —OR^{8a}, (CH₂)ₑC(═O)R^{8a}, (CH₂)ₑC(═O)OR^{8a}, —(CH₂)ₑC(═O)NR^{8a}R^{8b}, —NR^{8a}R^{8b}, —(CH₂)ₑOR^{8a}, —(CH₂)ₑNR^{8a}R^{8b}, and (CH₂)ₑS(═O)_{f}R¹¹;
wherein R² is selected from the group consisting of hydrido, hydroxyl, amino, C₁₋₈ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₁₋₂ aryl, and 5- to 12-membered heteroaryl;
wherein R⁴ is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R⁵ is selected from the group consisting of C₁₋₁₄ alkyl, C₃₋₁₂ cycloalkyl, C₃₋₁₂ cycloalkenyl, 3- to 12-membered heterocycloalkyl, 3- to 12-membered heterocycloalkenyl, 5- to 12-membered heteroaryl, and C₅₋₁₂ aryl, wherein R⁵ may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, C₅₋₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{7b}, R^{8a}, R^{8b}, R^{9a}, R^{9b}, R^{9c}, and R¹⁵ are independently selected from the group consisting of hydrido, C₁₋₆ alkyl, C₅₋₁₂ aryl, 5- to 12-membered heteroaryl, C₄₋₁₈, aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₂₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C, alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R¹⁰ is selected from the group consisting of C₃₋₁₂ cycloalkyl, C₃₋₁₂ cycloalkenyl, C₅₋₁₂ aryl, 3- to 12-membered heterocycloalkenyl, 5- to 12-membered heteroaryl, and C₂₋₆ alkenyl, wherein R¹⁰ is optionally substituted with one or more substituents selected from the group consisting of R^{13a};
wherein R¹¹, R^{12a}, and R^{12b} are independently selected from the group consisting of hydrido, C₁₋₂ aryl, 5- to 12-membered heteroaryl, 4- to 18-membered heteroaralkyl, C₁₋₆ alkyl, C₁₋₆ haloalkyl, C₂₋₆ alkenyl, C₂₋₆ alkynyl, C₁₋₆ hydroxyalkyl, C₁₋₆ aminoalkyl, C₂₋₁₂ alkylaminoalkyl, N-N-di(C₁₋₆ alkyl)amino(C₁₋₆ alkyl), C₁₋₆ alkoxy, C₂₋₁₂ alkoxyalkyl, 3- to 12-membered heterocycloalkyl, 3- to 12-membered heterocycloalkenyl, C₃₋₁₂ cycloalkyl, C₄₋₁₈ cycloalkylalkyl, C₄₋₁, aralkyl, and C₄₋₁₈ aralkylamino, wherein said aryl is optionally substituted with one or more radicals selected from the group consisting of C₁₋₆ alkyl, C₁₋₆ aminoalkyl, C₁₋₆ alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R¹³a and R^{13b} are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₄ alkoxycarbonyl, C₂₋₁₂ alkoxyalkyl, C₂₋₆ alkenylamino, C₂₋₆ alkynylamino, C₂₋₆ alkenyl, C₂, alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{14a} and R^{14b} are independently selected from the group consisting of hydrido, C₁₋₆ alkyl, 5- to 12-membered heteroaryl, 3- to 12-membered heterocycloalkenyl, C₁₋₆ haloalkyl, C₄₋₁₈ aralkylamino, 4- to 18-membered heteroaralkyl, C₅₋₁₂ aryl, and C₄₋₁₈ aralkyl, wherein said aryl, heteroaryl, heterocycloalkenyl, and aralkyl moieties are optionally substituted with one or more substituents selected from the group consisting of C₁₋₆ alkyl, C₁₋₆ alkoxy, halo, C₁₋₆ haloalkyl, cyano, C₁₋₆ haloalkoxy, C₂₋₁₀ acyl, carboxyl, hydroxy, C₁₋₆ hydroxyalkoxy, phenoxy, benzyloxy, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

In one particularly preferred embodiment, the compound of Formula III is a compound wherein A is selected from the group consisting of cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein A may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}, ——R^{7a}C(═O)—, —OR^{7a}C(═O)—, ethynylene, propynylene, butynylene, and pentynylene, wherein said R⁷, ethynylene, propynylene, butynylene, and pentynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, methylene, ethylene, propylene, butylene, pentylene, hexylene, ethenylene, propenylene, butenylene, pentenylene, ethynylene, propynylene, butynylene, and pentynylene, wherein said R⁷, methylene, ethylene, propylene, butylene, pentylene, hexylene, ethenylene, propenylene, butenylene, pentenylene, ethynylene, propynylene, butynylene, and pentynylene moieties may be substituted by one or more substituents independently selected from R¹⁵;
wherein R¹ is selected from the group consisting of hydrido, cyano, methyl, ethyl, propyl, butyl, pentyl, hexyl, ethenyl, propenyl, butenyl, pentenyl, chloro, fluoro, bromo, iodo, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, cyanomethyl, cyanoethyl, cyanopropyl, cyanobutyl, cyanopentyl, cyanohexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, —(CH₂)ₑOR^{8a}, —(CH₂)ₑNR^{8a}R^{8b}, and —(CH₂)ₑS(═O)_{f}R¹¹;
wherein R² is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R⁴ is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R⁵ is selected from the group consisting of methyl, ethyl, propyl, butyl, pentyl, hexyl, cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and phenyl, biphenyl, naphthyl, indenyl, wherein R⁵ may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R¹³a, and —C(═O)NR^{12a}R^{12b};
wherein R^{7a} is selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R^{7b}, R^{8a}, R^{8b}, R^{9a}, R^{9b}, R^{9c}, and R¹⁵ are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R¹⁰ is selected from the group consisting of cyanomethyl, cyanoethyl, cyanopropyl, cyanobutyl, cyanopentyl, cyanohexyl, cyclopropenyl, cyclobutenyl, cyclopentenyl, cyclohexenyl, phenyl, biphenyl, naphthyl, indenyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, ethenyl, propenyl, butenyl, and pentenyl, wherein R¹⁰ is optionally substituted with one or more substituents selected from the group consisting of R^{13a};
wherein R¹¹, R^{12a}, and R^{12b} are independently selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, methyl, ethyl, propyl, butyl, pentyl, hexyl, chloromethyl, dichloromethyl, trichloromethyl, fluoromethyl, difluoromethyl, trifluoromethyl, ethenyl, propenyl, butenyl, pentenyl, ethynyl, propynyl, butynyl, pentynyl, hydroxymethyl, hydroxyethyl, hydroxypropyl, hydroxybutyl, hydroxypentyl, hydroxyhexyl, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, N,N-dimethylaminomethyl, N,N-dimethylaminoethyl, N-methyl-N-ethylaminomethyl, N-methyl-N-ethylaminoethyl, N-methyl-N-propylaminomethyl, N-methyl-N-propylaminoethyl, N,N-diethylaminomethyl, N,N-diethylaminoethyl, N-ethyl-N-propylaminomethyl, N-ethyl-N-propylaminoethyl, N,N-dipropylaminomethyl, N,N-dipropylaminoethyl, methoxy, ethoxy, propoxy, butoxy, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl, cyclohexylethyl, benzyl, phenylethyl, benzylamino, and phenylethylamino, wherein said phenyl, biphenyl, naphthyl, indenyl is optionally substituted with one or more radicals selected from the group consisting of methyl, ethyl, propyl, butyl, pentyl, hexyl, aminomethyl, aminoethyl, aminopropyl, aminobutyl, aminopentyl, aminohexyl, methoxy, ethoxy, propoxy, butoxy and chloro, fluoro, bromo, iodo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{14a} and R^{14b} are independently selected from the group consisting of hydrido, methyl, ethyl, propyl, butyl, pentyl, hexyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, chloromethyl, dichloromethyl, trichloromethyl, fluoromethyl, difluoromethyl, trifluoromethyl, benzylamino, phenylethylamino, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, phenyl, biphenyl, naphthyl, indenyl, benzyl, and phenylethyl, wherein said phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, benzyl, and phenylethyl moieties are optionally substituted with one or more substituents selected from the group consisting of methyl, ethyl, propyl, butyl, pentyl, hexyl, methoxy, ethoxy, propoxy, butoxy, chloro, fluoro, bromo, iodo, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, benzyloxy, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

In a preferred embodiment, the compound of Formula I is a compound of Formula IV: 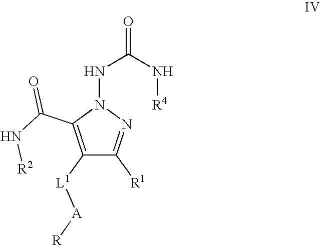
wherein L¹, L², A, R, R¹, R², and R⁴ are as defined above for Formula III.

In a preferred embodiment, the compound of Formula I is a compound of Formula V: 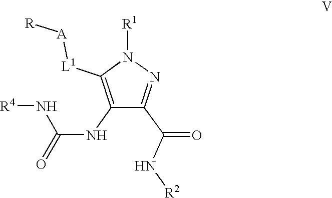
wherein L¹, A, R, R¹, R², and R⁴ are as defined above for Formula III.

In a preferred embodiment, the compound of Formula I is a compound of Formula VI: 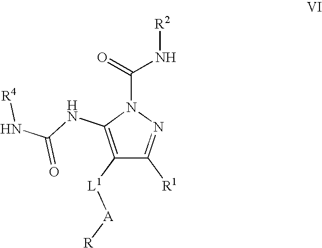
wherein L¹, A, R, R¹, R², and R⁴ are as defined above for Formula III.

In a preferred embodiment, the compound of Formula I is a compound of Formula VII: 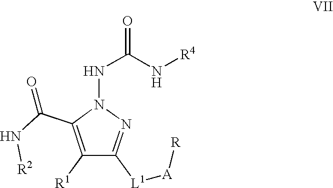
wherein L¹, A, R, R¹, R², and R⁴ are as defined above for Formula III.

In a preferred embodiment, the compound of Formula I is a compound of Formula VIIIl: 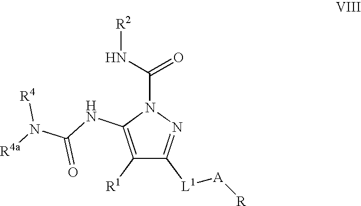
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R is selected from the group consisting of hydrido and -L²R⁵;
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}—, —R^{7a}C(═O)—, —OR^{7a}C(═O)—, and alkynylene, wherein said R^{7a}, alkylene, alkenylene, and alkynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R^{7b}, —C(═O)R^{7b}, —R^{7b}C(═O)—, —OR^{7b}C(═O)—, alkylene, alkenylene, and alkynylene, wherein said R^{7b}, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{b}, —(CH₂)ₑOR^{8a}, —(CH₂)ₑNR^{8a}R^{8b}, and —(CH₂)ₑS(═O)_{f}R¹¹, or R¹ together with R^{4a} and the atoms to which they are attached form a heterocyclic ring moiety having the structure: 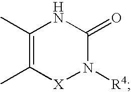
wherein R² is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R^{4a} is selected from the group consisting of hydrido, hydroxyl, alkoxy, alkyl, haloalkyl, aryl, and heteroaryl, or R^{4a} together with R¹ and the atoms to which they are attached form a heterocyclic ring having the structure: 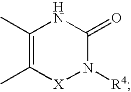
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl, and wherein R⁴, when R¹ together with R^{4a} and the atoms to which they are attached form a heterocyclic ring having 6 or more members, may form a double bond between the nitrogen to which R⁴ is attached and X;
wherein X is selected from the group consisting of a bond, —(CHR^{9a})ₓ(CHR^{9b})_{y}(CHR^{9c})₂, —NR^{9a}—, —CR^{9a}═CR^{9b}—(CHR^{9c})_{g}—, —(CH₂)ₕO—, —(CH₂)ₕS—, —NR^{9a}NR^{9b}, —N═N—, —ONR^{9a}—, SNR^{9a}—, —CH═CH—CH₂—, —CH═CH—NR^{9a}—, —CH₂—O—CH₂, —CH₂—S—CH₂—, —CH₂—N═N—, —NR^{9a}O—CH₂—, —CH₂—NR^{9a} S, and —NR⁹a—S—CH₂—;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R^{7a} is selected from the group consisting of hydrido, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{7b}, R^{8a}, R^{8b}, R^{9a}, R^{9b}, R^{9c}, and R⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R¹³a and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂70, N, and NR^{13a};
wherein a, d, and e are independently selected from the group consisting of 0, 1, 2, 3, 4, 5, 6, 7, or 8;
wherein b, c, f, and h are independently selected from the group consising of 0, 1, or 2;
wherein g is 0 or 1;
wherein x, y, and z are independently 0, 1, 2, or 3, and wherein x+y+z<4;
or a pharmaceutically-acceptable salt thereof.

In one particularly preferred embodiment, the compound of Formula VIII is a compound wherein A is selected from the group consisting of C₃₋₁₂ cycloalkyl, C₃₋₁₂ cycloalkenyl, 3- to 12-membered heterocycloalkyl, 3- to 12-membered heterocycloalkenyl, C₅₋₁₂ aryl, and 5- to 12-membered heteroaryl, wherein A may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O))R^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}, —C(═O)R^{7a}—, —R^{7a}C(═O)—, —OR^{7a}C(═O)—, and C₂₋₆ alkynylene, wherein said R⁷ and alkynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, C₁₋₆ alkylene, C₂₋₆ alkenylene, and C₂₋₆ alkynylene, wherein said R⁷, alkylene, alkenylene, and alkynylene moieties may be substituted by one or more substituents independently selected from R¹⁵;
wherein R¹ is selected from the group consisting of hydrido, cyano, C₁₋₆ alkyl, C₂₋₆ alkenyl, halo, C₁₋₆ haloalkyl, C₁₋₆ hydroxyalkyl, cyano(C₁₋₆ alkyl), C₅₋₁₂ aryl, 5- to 12-membered heteroaryl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}NR^{8b}, (CH₂)ₑOR^{8a}, —(CH₂)ₑNR^{8a}R^{8b}, and (CH₂)ₑS(═O)_{f}R¹, or R¹ together with R^{4a} and the atoms to which they are attached form a 5- to 8-membered heterocyclic ring moiety having the structure: 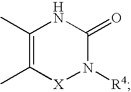
wherein R² is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R^{4a} is selected from the group consisting of hydrido, hydroxyl, C₁₋₆ alkoxy, C₁₋₆, alkyl, C₁₋₆ haloalkyl, C₅₋₁₂ aryl, and 5- to 12-membered heteroaryl, or R^{4a} together with R¹ and the atoms to which they are attached form a 5- to 8-membered heterocyclic ring having the structure: 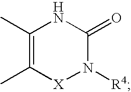
wherein R⁴ is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of C₁₋₆ alkyl, C₃₋₁₂ cycloalkyl, C₃₋₁₂ cycloalkenyl, 3- to 12-membered heterocycloalkyl, 3- to 12-membered heterocycloalkenyl, 5- to 12-membered heteroaryl, and C₅₋₁₂ aryl, wherein R⁵ may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆, haloalkyl, C₁₋₆ hydroxyalkyl, C₁₋₆ haloalkoxy, nitro, C₂₋₁₀ acylamino, R¹⁰, —OR¹¹, —C(═N)NR^{12a}R^{12b}, —NR^{12a}—N(═CR^{13a}R^{13b}), —(CH₂)ₑOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═)OR^{13a}, —NR^{13a}SO₂R^{14a}, —NR^{13a}SO₂NR¹⁴R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{7b}, R^{8a}, R^{8b}, R^{9a}, R^{9b}, R^{9c}, and R¹⁵ are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R¹⁰ is selected from the group consisting of C₃₋₁₂ cycloalkyl, C₃₋₁₂ cycloalkenyl, C₅₋₁₂ aryl, 3- to 12-membered heterocycloalkenyl, 5- to 12-membered heteroaryl, and C₂₋₆ alkenyl, wherein R¹⁰ is optionally substituted with one or more substituents selected from the group consisting of R^{13a};
wherein R¹¹, R^{12a}, and R^{12b} are independently selected from the group consisting of hydrido, C₅₋₁₂ aryl, 5- to 12-membered heteroaryl, 4- to 18-membered heteroaralkyl, C₁₋₆ alkyl, C₁₋₆ haloalkyl, C₂₋₆ alkenyl, C₂₋₆ alkynyl, C₁₋₆ hydroxyalkyl, C₁₋₆ aminoalkyl, C₂₋₁₂ alkylaminoalkyl, N-N-di(C₁₋₆ alkyl)amino(C₆alkyl), C₁₋₆ alkoxy, C₂₋₁₂ alkoxyalkyl, 3- to 12-membered heterocycloalkyl, 3- to 12-membered heterocycloalkenyl, C₃₋₁₂ cycloalkyl, C₄₋₁₈ cycloalkylalkyl, C₄₋₁₈ aralkyl, and C₄₋₁₈ aralkylamino, wherein said aryl is optionally substituted with one or more radicals selected from the group consisting of C₁₋₆ alkyl, C₁₋₆ aminoalkyl, C₁₋₆ alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{14a} and R^{14b} are independently selected from the group consisting of hydrido, C₁₋₆ alkyl, 5- to 12-membered heteroaryl, 3- to 12-membered heterocycloalkenyl, C₁₋₆ haloalkyl, C₄₋₁₈ aralkylamino, 4- to 18-membered heteroaralkyl, C₅₋₁₂ aryl, and C₄₋₁₈ aralkyl, wherein said aryl, heteroaryl, heterocycloalkenyl, and aralkyl moieties are optionally substituted with one or more substituents selected from the group consisting of C₁₋₆ alkyl, C₁₋₆ alkoxy, halo, C₁₋₆ haloalkyl, cyano, C₁₋₆ haloalkoxy, C₂₋₁₀ acyl, carboxyl, hydroxy, C₁₋₆ hydroxyalkoxy, phenoxy, benzyloxy, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl, wherein R^{14a} and R^{14b} may be taken together to form a 3- to 7-membered carbocyclic ring having 1 to 3 heteroatoms selected from S, SO, SO2, O, N, and NR^{13a};
or a pharmaceutically-acceptable salt thereof.

In an even more particularly preferred embodiment, the compound of Formula VIII is a compound wherein A is selected from the group consisting of cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein A may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentyinaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}, C(═O)R^{7a}, —R^{7a}C(═O)—, —OR^{7a}C(═O)—, ethynylene, propynylene, butynylene, and pentynylene, wherein said R⁷, ethynylene, propynylene, butynylene, and pentynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, methylene, ethylene, propylene, butylene, pentylene, hexylene, ethenylene, propenylene, butenylene, pentenylene, ethynylene, propynylene, butynylene, and pentynylene, wherein said R⁷, methylene, ethylene, propylene, butylene, pentylene, hexylene, ethenylene, propenylene, butenylene, pentenylene, ethynylene, propynylene, butynylene, and pentynylene moieties may be substituted by one or more substituents independently selected from R¹⁵;
wherein R¹ is selected from the group consisting of hydrido, cyano, methyl, ethyl, propyl, butyl, pentyl, hexyl, ethenyl, propenyl, butenyl, pentenyl, chloro, fluoro, bromo, iodo, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, cyanomethyl, cyanoethyl, cyanopropyl, cyanobutyl, cyanopentyl, cyanohexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, (CH₂)ₑOR^{8a}, —(CH₂)ₑNR^{8a}R^{8b}, and —(CH₂)ₐS(═O)_{f}R¹¹, or R¹ together with R^{4a} and the atoms to which they are attached form a 5- to 8-membered heterocyclic ring moiety having the structure: 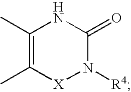
wherein R² is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonyl propyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{4a} is selected from the group consisting of hydrido, hydroxyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, or R^{4a} together with R¹ and the atoms to which they are attached form a 5- to 8-membered heterocyclic ring having the structure: 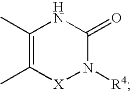
wherein R⁴ is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of methyl, ethyl, propyl, butyl, pentyl, hexyl, cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and phenyl, biphenyl, naphthyl, indenyl, wherein R⁵ may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R^{7b}, R^{8a}, R^{8b}, R^{9a}, R^{9b}, R^{9c}, and R¹⁵ are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R¹⁰ is selected from the group consisting of cyanomethyl, cyanoethyl, cyanopropyl, cyanobutyl, cyanopentyl, cyanohexyl, cyclopropenyl, cyclobutenyl, cyclopentenyl, cyclohexenyl, phenyl, biphenyl, naphthyl, indenyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, ethenyl, propenyl, butenyl, and pentenyl, wherein R¹⁰ is optionally substituted with one or more substituents selected from the group consisting of R^{13a};
wherein R¹¹, R^{12a}, and R^{12b} are independently selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, methyl, ethyl, propyl, butyl, pentyl, hexyl, chloromethyl, dichloromethyl, trichloromethyl, fluoromethyl, difluoromethyl, trifluoromethyl, ethenyl, propenyl, butenyl, pentenyl, ethynyl, propynyl, butynyl, pentynyl, hydroxymethyl, hydroxyethyl, hydroxypropyl, hydroxybutyl, hydroxypentyl, hydroxyhexyl, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, N,N-dimethylaminomethyl, N,N-dimethylaminoethyl, N-methyl-N-ethylaminomethyl, N-methyl-N-ethylaminoethyl, N-methyl-N-propylaminomethyl, N-methyl-N-propylaminoethyl, N,N-diethylaminomethyl, N,N-diethylaminoethyl, N-ethyl-N-propylaminomethyl, N-ethyl-N-propylaminoethyl, N,N-dipropylaminomethyl, N,N-dipropylaminoethyl, methoxy, ethoxy, propoxy, butoxy, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl, cyclohexylethyl, benzyl, phenylethyl, benzylamino, and phenylethylamino, wherein said phenyl, biphenyl, naphthyl, indenyl is optionally substituted with one or more radicals selected from the group consisting of methyl, ethyl, propyl, butyl, pentyl, hexyl, aminomethyl, aminoethyl, aminopropyl, aminobutyl, aminopentyl, aminohexyl, methoxy, ethoxy, propoxy, butoxy and chloro, fluoro, bromo, iodo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{14a} and R^{14b} are independently selected from the group consisting of hydrido, methyl, ethyl, propyl, butyl, pentyl, hexyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, chloromethyl, dichloromethyl, trichloromethyl, fluoromethyl, difluoromethyl, trifluoromethyl, benzylamino, phenylethylamino, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, phenyl, biphenyl, naphthyl, indenyl, benzyl, and phenylethyl, wherein said phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, benzyl, and phenylethyl moieties are optionally substituted with one or more substituents selected from the group consisting of methyl, ethyl, propyl, butyl, pentyl, hexyl, methoxy, ethoxy, propoxy, butoxy, chloro, fluoro, bromo, iodo, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, benzyloxy, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

In a preferred embodiment, the compound of Formula I is a compound of Formula IX: 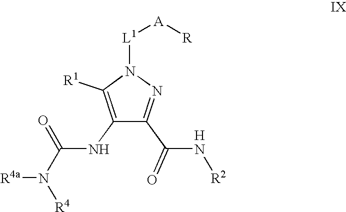
wherein L¹, A, R, R¹, R², R⁴, and R⁴′ are as defined above for Formula VIII.

In a preferred embodiment, the compound of Formula I is a compound of Formula X: 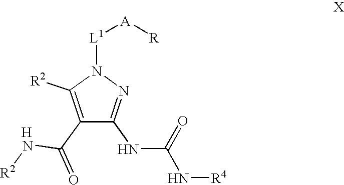
wherein L¹, A, R, R¹, R², and R⁴ are as defined above for Formula III.

In a preferred embodiment, the compound of Formula I is selected from the group of compounds consisting of the compounds shown in Table I below:

TABLE I

| Example | Name | Structure |
|---|---|---|
| 1 | 4-[(aminocarbonyl)amino]-1-[4- bromo-3-(trifluoromethyl)phenyl]- 1H-pyrazole-3-carboxamide | 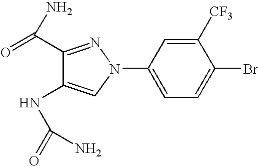 |
| 3 | 4-[(aminocarbonyl)amino]-1-(4- bromo-3-ethoxyphenyl)-1H- pyrazole-3-carboxamide | 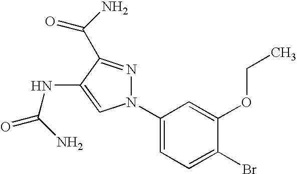 |
| 4 | 4-[(aminocarbonyl)amino]-1-(4- iodo-2-methylphenyl)-1H-pyrazole- 3-carboxamide | 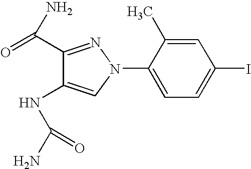 |
| 5 | 4-[(aminocarbonyl)amino]-1-(3′- hydroxy-3-methyl-1,1′-biphenyl-4- yl)-1H-pyrazole-3-carboxamide | 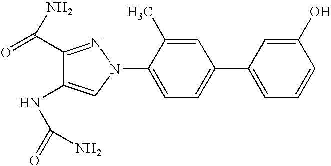 |
| 6 | 4-[(aminocarbonyl)amino]-1-(3- chloro-4-iodophenyl)-1H-pyrazole- 3-carboxamide | 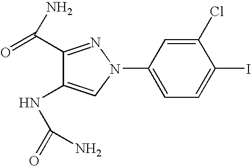 |
| 7 | 4-[(aminocarbonyl)amino]-1-(2- chloro-3′-hydroxy-1,1′-biphenyl-4- yl)-1H-pyrazole-3-carboxamide | 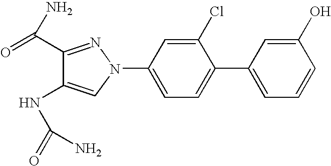 |
| 8 | 4-[(aminocarbonyl)amino]-1-(4- iodo-3-methylphenyl)-1H-pyrazole- 3-carboxamide | 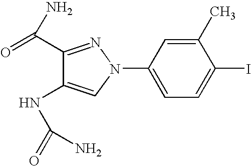 |
| 9 | 4-[(aminocarbonyl)amino]-1-[4- (morpholin-4-yl-carbonyl)phenyl]- 1H-pyrazole-3-carboxamide | 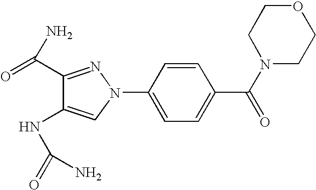 |
| 10 | 4-[(aminocarbonyl)amino]-1-(4- methoxyphenyl)-1H-pyrazole-3- carboxamide trifluoroacetate | 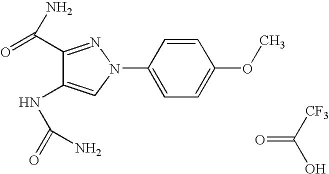 |
| 11 | 4-[(aminocarbonyl)amino]-1-[3- (3,4-dimethoxybenzyl)phenyl]-1H- pyrazole-3-carboxamide | 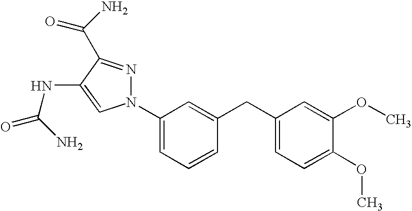 |
| 12 | 4-[(aminocarbonothioyl)amino]-1- (3-bromophenyl)-1H-pyrazole-3- carboxamide | 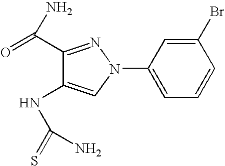 |
| 13 | 4-[(aminocarbonyl)amino]-1-[3-(2- chloropyridin-4-yl)phenyl]-1H- pyrazole-3-carboxamide | 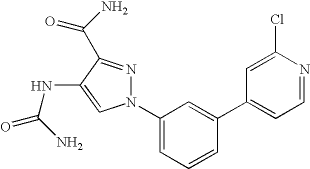 |
| 14 | 4-[(aminocarbonyl)amino]-1-(4- methoxyphenyl)-1H-pyrazole-3- carboxamide trifluoroacetate | 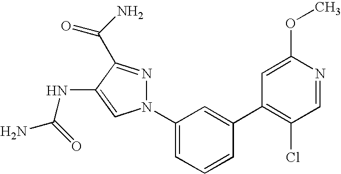 |
| 15 | 1-(3-bromophenyl)-4- {[(methylamino)carbonyl]amino}- 1H-pyrazole-3-carboxamide | 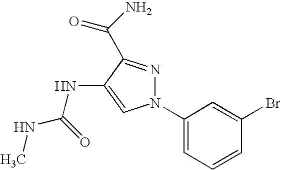 |
| 16 | 1-(4-bromophenyl)-4- {[(methylamino)carbonyl]amino}- 1H-pyrazole-3-carboxamide | 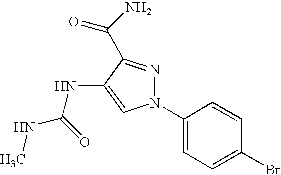 |
| 17 | 1-(4-iodophenyl)-4- {[(methylamino)carbonyl]amino}- 1H-pyrazole-3-carboxamide | 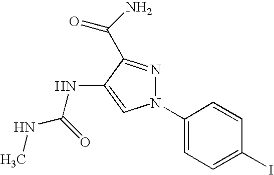 |
| 18 | 4-[(aminocarbonyl)amino]-1-(4- methylphenyl)-1H-pyrazole-3- carboxamide | 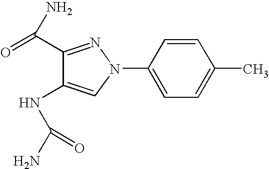 |
| 19 | 4-[(aminocarbonyl)amino]-1-(4- bromophenyl)-1H-pyrazole-3- carboxamide | 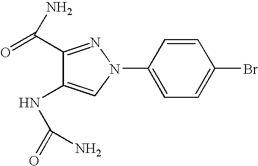 |
| 20 | 4-[(aminocarbonyl)amino]-1-(3- bromophenyl)-1H-pyrazole-3- carboxamide | 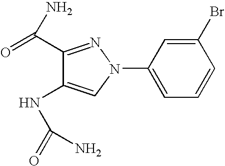 |
| 21 | 4-[(aminocarbonyl)amino]-1-(4- pyridin-3-ylphenyl)-1H-pyrazole-3- carboxamide | 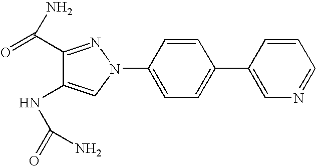 |
| 22 | 4-[(aminocarbonyl)amino]-1-(3′- hydroxy-1,1′-biphenyl-4-yl)-1H- pyrazole-3-carboxamide | 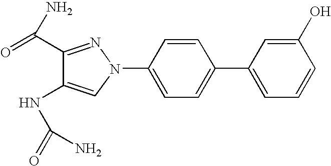 |
| 23 | 4-[(aminocarbonyl)amino]-1-(2′- hydroxy-1,1′-biphenyl-4-yl)-1H- pyrazole-3-carboxamide | 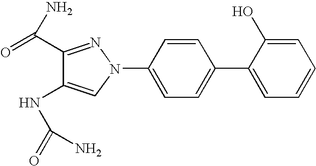 |
| 24 | 4-[(aminocarbonyl)amino]-1-[3′- (trifluoromethoxy)-1,1′-biphenyl-4- yl]-1H-pyrazole-3-carboxamide | 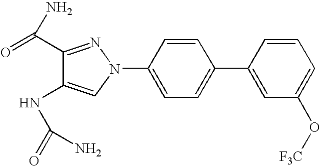 |
| 25 | 4-[(aminocarbonyl)amino]-1-(4′- fluoro-1,1′-biphenyl-4-yl)-1H- pyrazole-3-carboxamide | 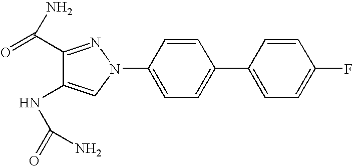 |
| 26 | 4-[(aminocarbonyl)amino]-1-[4-(1H- pyrrol-2-yl)phenyl]-1H-pyrazole-3- carboxamide | 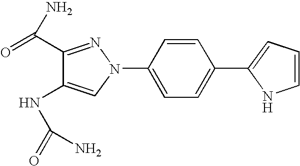 |
| 27 | 4-[(aminocarbonyl)amino]-1-[3′- (cyanomethyl)-1,1′-biphenyl-4-yl]- 1H-pyrazole-3-carboxamide | 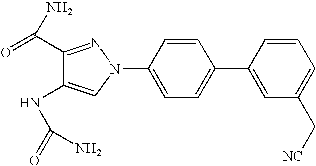 |
| 28 | 4-[(aminocarbonyl)amino]-1-[3′- (aminomethyl)-1,1′-biphenyl-4-yl]- 1H-pyrazole-3-carboxamide | 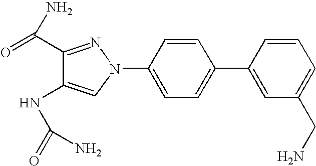 |
| 29 | methyl 2-amino-4′-{3- (aminocarbonyl)-4- [(aminocarbonyl)amino]-1H- pyrazol-1-yl}-1,1′-biphenyl-4- carboxylate | 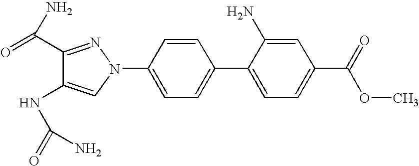 |
| 30 | 4-[(aminocarbonyl)amino]-1-(2′- fluoro-1,1′-biphenyl-4-yl)-1H- pyrazole-3-carboxamide | 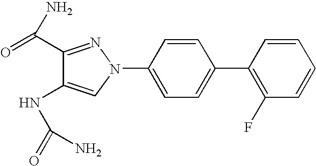 |
| 31 | 4-[(aminocarbonyl)amino]-1-{3′- [(tert-butylamino)carbonyl]-1,1′- biphenyl-4-yl}-1H-pyrazole-3- carboxamide | 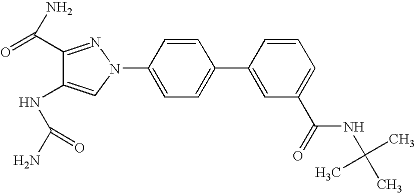 |
| 32 | 4-[(aminocarbonyl)amino]-1-{3′- [(E)-2-cyanovinyl]-1,1′-biphenyl-4- yl}-1H-pyrazole-3-carboxamide | 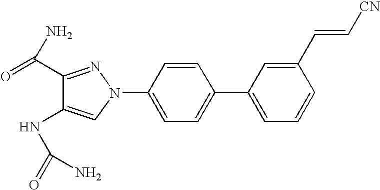 |
| 33 | 4-[(aminocarbonyl)amino]-1-{4′- [(pyridin-2-ylamino)carbonyl]-1,1′- biphenyl-4-yl}-1H-pyrazole-3- carboxamide | 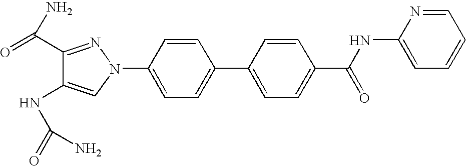 |
| 34 | 4-[(aminocarbonyl)amino]-1-[4′- (morpholin-4-ylmethyl)-1,1′- biphenyl-4-yl]-1H-pyrazole-3- carboxamide | 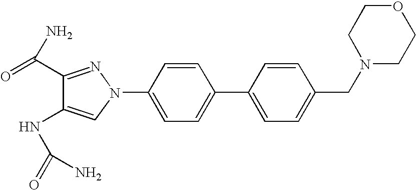 |
| 35 | 4-[(aminocarbonyl)amino]-1-{4′- [(benzylamino)carbonyl]-1,1′- biphenyl-4-yl}-1H-pyrazole-3- carboxamide | 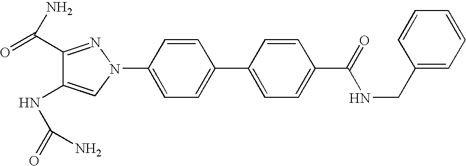 |
| 36 | 4-[(aminocarbonyl)amino]-1-(4′- hydroxy-1,1′-biphenyl-4-yl)-1H- pyrazole-3-carboxamide | 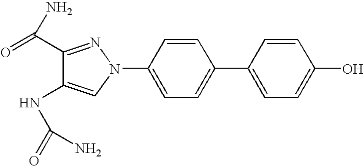 |
| 37 | 4-[(aminocarbonyl)amino]-1-[4′- (cyanomethyl)-1,1′-biphenyl-4-yl]- 1H-pyrazole-3-carboxamide | 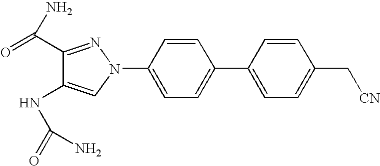 |
| 38 | 4-[(aminocarbonyl)amino]-1-[4′- (hydroxymethyl)-1,1′-biphenyl-4-yl]- 1H-pyrazole-3-carboxamide | 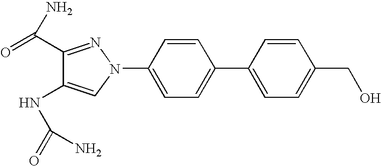 |
| 39 | 4-[(aminocarbonyl)amino]-1-[3′- (hydroxymethyl)-1,1′-biphenyl-4-yl]- 1H-pyrazole-3-carboxamide | 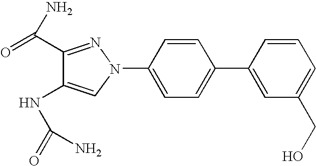 |
| 40 | 4-[(aminocarbonyl)amino]-1-(4- pyridin-4-ylphenyl)-1H-pyrazole-3- carboxamide | 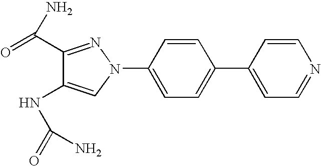 |
| 41 | 4-[(aminocarbonyl)amino]-1-(3′- fluoro-1,1′-biphenyl-3-yl)-1H- pyrazole-3-carboxamide | 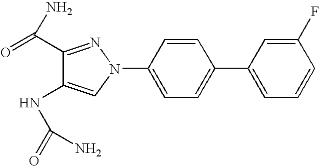 |
| 42 | 4-[(aminocarbonyl)amino]-1-(4′- fluoro-1,1′-biphenyl-3-yl)-1H- pyrazole-3-carboxamide | 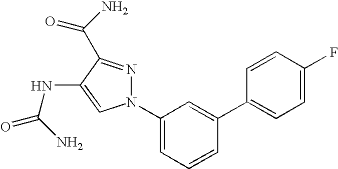 |
| 43 | 4-[(aminocarbonyl)amino]-1-(3- thien-3-ylphenyl)-1H-pyrazole-3- carboxamide | 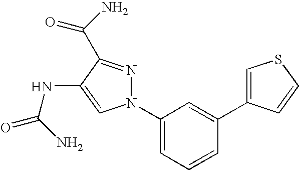 |
| 44 | 4-[(aminocarbonyl)amino]-1-[4′- (cyanomethyl)-1,1′-biphenyl-3-yl]- 1H-pyrazole-3-carboxamide | 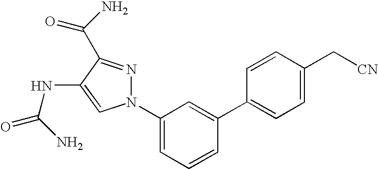 |
| 45 | 4-[(aminocarbonyl)amino]-1-(3- pyridin-4-ylphenyl)-1H-pyrazole-3- carboxamide | 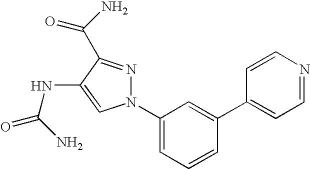 |
| 46 | 4-[(aminocarbonyl)amino]-1-(3- pyridin-4-ylphenyl)-1H-pyrazole-3- carboxamide | 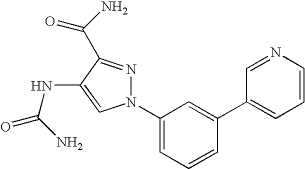 |
| 47 | 4-[(aminocarbonyl)amino]-1-(4′- hydroxy-1,1 ′-biphenyl-3-yl)-1H- pyrazole-3-carboxamide | 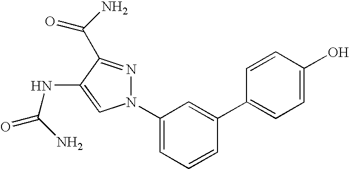 |
| 48 | 4-[(aminocarbonyl)amino]-1-[4′- (hydroxymethyl)-1,1′-biphenyl-3-yl]- 1H-pyrazole-3-carboxamide | 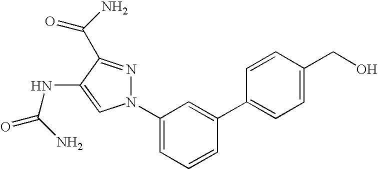 |
| 49 | 4-[(aminocarbonyl)amino]-1-[3′- (hydroxymethyl)-1,1′-biphenyl-3-yl]- 1H-pyrazole-3-carboxamide | 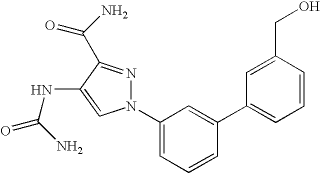 |
| 50 | 1-(3′-amino-1,1′-biphenyl-3-yl)-4- [(aminocarbonyl)amino]-1H- pyrazole-3-carboxamide | 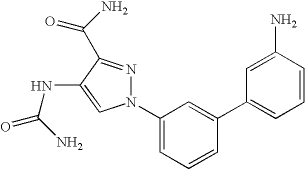 |
| 51 | 4-[(aminocarbonyl)amino]-1-[4- (morpholin-4-ylmethyl)-1,1′- biphenyl-3-yl]-1H-pyrazole-3- carboxamide | 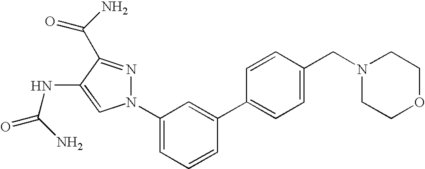 |
| 52 | 4-[(aminocarbonyl)amino]-1-[4′- (aminomethyl)-1,1′-biphenyl-3-yl]- 1H-pyrazole-3-carboxamide | 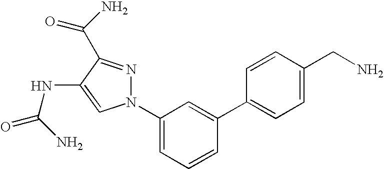 |
| 53 | 1-(4′-acetyl-1,1′-biphenyl-3-yl)-4- [(aminocarbonyl)amino]-1H- pyrazole-3-carboxamide | 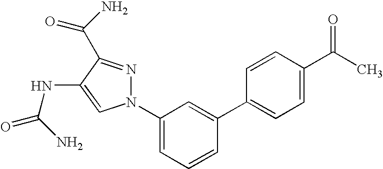 |
| 54 | 1-(4-amino-1,1′-biphenyl-3-yl)-4- [(aminocarbonyl)amino]-1H- pyrazole-3-carboxamide | 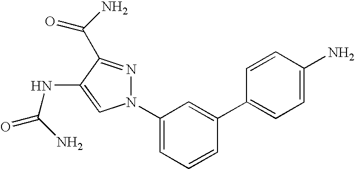 |
| 55 | 1-[4′-(acetylamino)-1,1′-biphenyl-3- yl]-4-[(aminocarbonyl)amino]-1H- pyrazole-3-carboxamide | 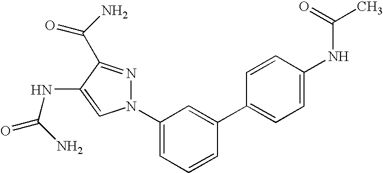 |
| 56 | 4-[(aminocarbonyl)amino]-1-(3,4′- dimethoxy-1,1′-biphenyl-3-yl)-1H- pyrazole-3-carboxamide | 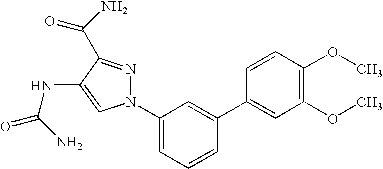 |
| 57 | 4-[(aminocarbonyl)amino]-1-[3-(1H- pyrazol-4-yl)phenyl]-1H-pyrazole-3- carboxamide | 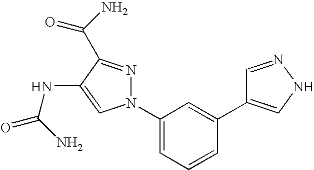 |
| 58 | 3′-{3-(aminocarbonyl)-4- [(aminocarbonyl)amino]-1H- pyrazol-1-yl}-1,1′-biphenyl-4- carboxylic acid | 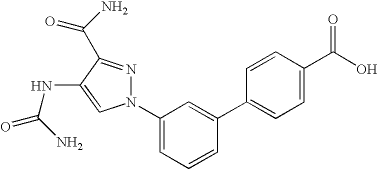 |
| 59 | 4-[(aminocarbonyl)amino]-1-{4′- [(methylsulfonyl)amino]-1,1′- biphenyl-3-yl}-1H-pyrazole-3- carboxamide | 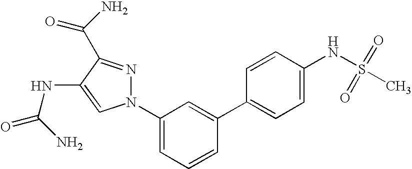 |
| 60 | 4-[(aminocarbonyl)amino]-1-(3- quinolin-5-ylphenyl)-1H-pyrazole-3- carboxamide | 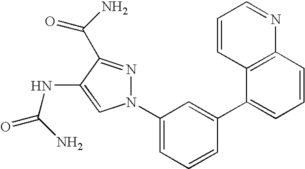 |
| 61 | 4-[(aminocarbonyl)aminol-1-[4′- (dimethylamino)-1,1′-biphenyl-3-yl]- 1H-pyrazole-3-carboxamide | 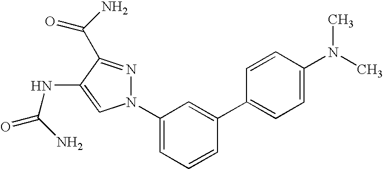 |
| 62 | 4-[(aminocarbonyl)amino]-1-[3- (1,3-benzodioxol-5-yl)phenyl]-1H- pyrazole-3-carboxamide | 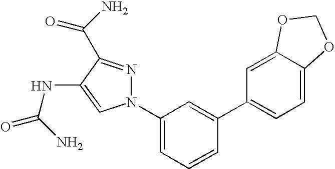 |
| 63 | 4-[(aminocarbonyl)amino]-1-[3-(2- methylpyridin-4-yl)phenyl]-1H- pyrazole-3-carboxamide | 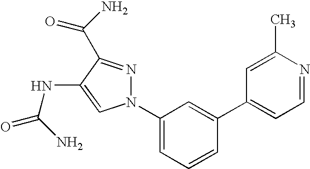 |
| 64 | 4-[(aminocarbonyl)amino]-1-[3-(4- methyl-3,4-dihydro-2H-1,4- benzoxazin-7-yl)phenyl]-1H- pyrazole-3-carboxamide | 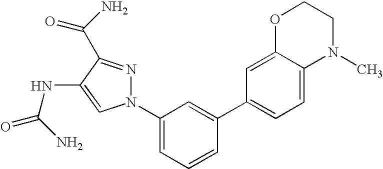 |
| 65 | 4-[(aminocarbonyl)amino]-1-[3-(1H- indol-5-yl)phenyl]-1H-pyrazole-3- carboxamide | 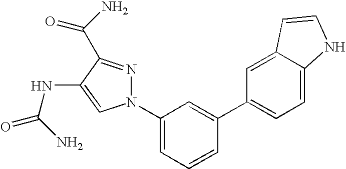 |
| 66 | 4-[(aminocarbonyl)amino]-1-(3- pyrimidin-5-ylphenyl)-1H-pyrazole- 3-carboxamide | 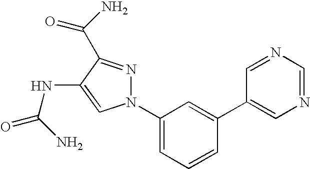 |
| 67 | 4-[(aminocarbonyl)amino]-1-{3- [(E)-2-phenylvinyl]phenyl}-1H- pyrazole-3-carboxamide | 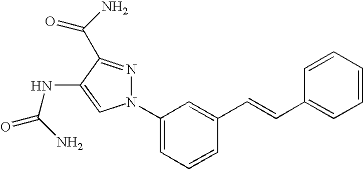 |
| 68 | 4-[(aminocarbonyl)amino]-1-[3-(1H- pyrrol-2-yl)phenyll-1H-pyrazole-3- carboxamide | 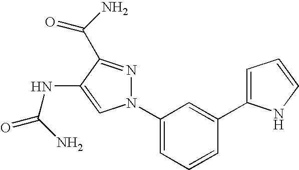 |
| 69 | 4-[(aminocarbonyl)amino]-1-[3-(3- methylpyridin-4-yl)phenyl]-1H- pyrazole-3-carboxamide | 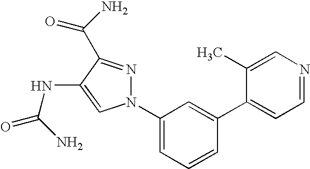 |
| 70 | 4-[(aminocarbonyl)amino]-1-[3- (3,5-dimethylisoxazol-4-yl)phenyl]- 1H-pyrazole-3-carboxamide | 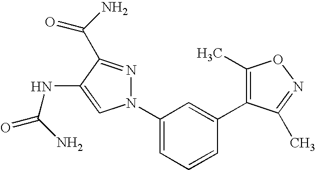 |
| 71 | 4-[(aminocarbonyl)amino]-1-(2′- hydroxy-1,1′-biphenyl-3-yl)-1H- pyrazole-3-carboxamide | 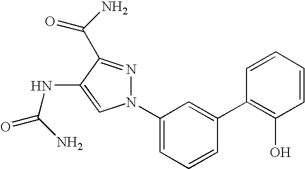 |
| 72 | 4-[(aminocarbonyl)amino]-1-(3′- hydroxy-1,1′-biphenyl-3-yl)-1H- pyrazole-3-carboxamide | 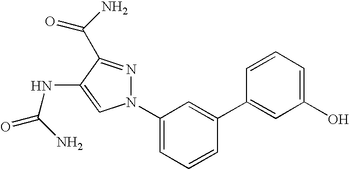 |
| 73 | 3′-{3-(aminocarbonyl)-4- [(aminocarbonyl)amino]-1H- pyrazol-1-yl}-1,1′-biphenyl-3- carboxylic acid | 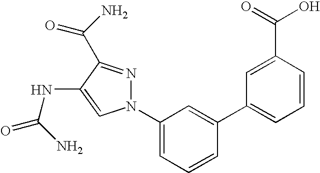 |
| 74 | 4-[(aminocarbonyl)amino]-1-{4′- [(methylamino)carbonyl]-1,1′- biphenyl-3-yl}-1H-pyrazole-3- carboxamide | 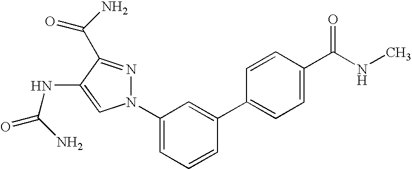 |
| 75 | 4-[(aminocarbonyl)amino]-1-{4′- [(propylamino)carbonyl]-1,1′- biphenyl-3-yl}-1H-pyrazole-3- carboxamide | 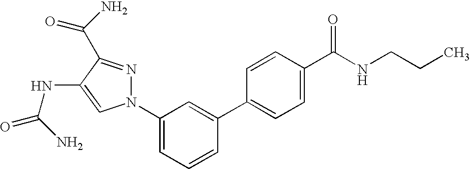 |
| 76 | 1-[3′-(acetylamino)-1,1′-biphenyl-3- yl]-4-[(aminocarbonyl)amino]-1H- pyrazole-3-carboxamide | 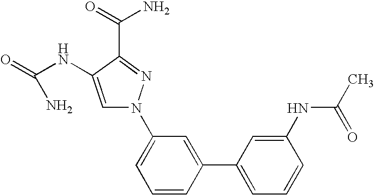 |
| 77 | 4-[(aminocarbonyl)amino]-1-(3′- amino-4′-methyl-1,1′-biphenyl-3-yl)- 1H-pyrazole-3-carboxamide | 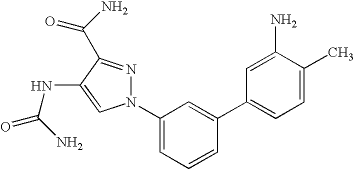 |
| 78 | 4-[(aminocarbonyl)amino]-1-[3′- (aminomethyl)-1,1′-biphenyl-3-yl]- 1H-pyrazole-3-carboxamide | 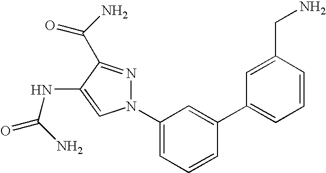 |
| 79 | 4-[(aminocarbonyl)amino]-1-[3′- (aminocarbonyl)-1,1′-biphenyl-3-yl]- 1H-pyrazole-3-carboxamide | 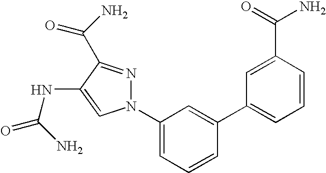 |
| 80 | 4-[(aminocarbonyl)amino]-1-{4′- [(cyclopropylamino)carbonyl]-1,1′- biphenyl-3-yl}-1H-pyrazole-3- carboxamide | 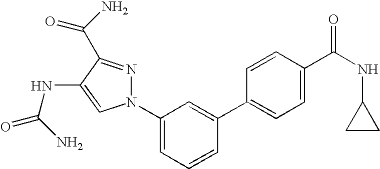 |
| 81 | 4-[(aminocarbonyl)amino]-1-{3′- [(cyclopropylamino)carbonyll-1,1′- biphenyl-3-yl}-1H-pyrazole-3- carboxamide | 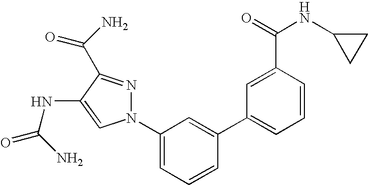 |
| 82 | 4-[(aminocarbonyl)amino]-1-(3′- {[(2-cyanoethyl)amino]carbonyl}- 1,1′-biphenyl-3-yl)-1H-pyrazole-3- carboxamide | 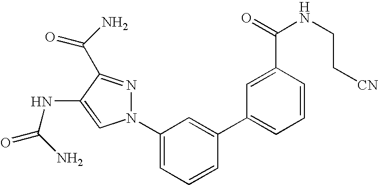 |
| 83 | 4-[(aminocarbonyl)amino]-1-{3′- [(methylamino)carbonyl]-1,1′- biphenyl-3-yl}-1H-pyrazole-3- carboxamide | 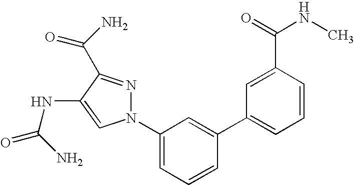 |
| 84 | 4-[(aminocarbonyl)amino]-1-(4′- {[(2-cyanoethyl)amino]carbonyl}- 1,1′-biphenyl-3-yl)-1H-pyrazole-3- carboxamide | 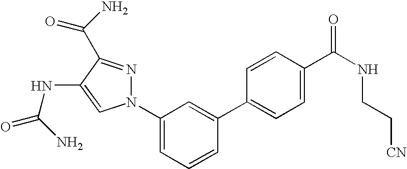 |
| 85 | N-[(3′-{3-(aminocarbonyl)-4- [(aminocarbonyl)amino]-1H- pyrazol-1-yl}-1,1′-biphenyl-4- yl)carbonyl]glycine | 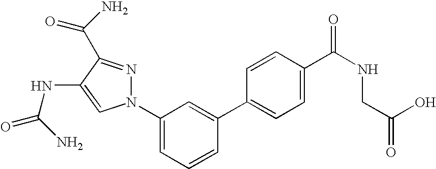 |
| 86 | 4-[(aminocarbonyl)amino]-1-[2′- (benzyloxy)-1,1′-biphenyl-3-yl]-1H- pyrazole-3-carboxamide | 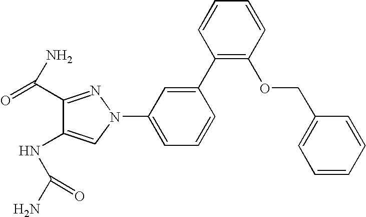 |
| 87 | 4-[(aminocarbonyl)aminol-1-[2′- (hydroxymethyl)-1,1′-biphenyl-3-yl]- 1H-pyrazole-3-carboxamide | 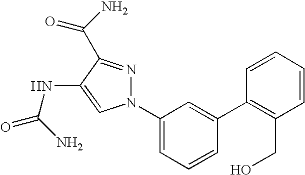 |
| 88 | 4-[(aminocarbonyl)amino]-1-(2′- methoxy-1,1′-biphenyl-3-yl)-1H- pyrazole-3-carboxamide | 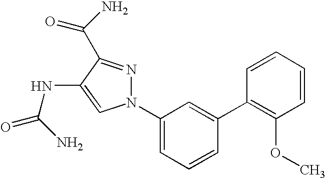 |
| 89 | 4-[(aminocarbonyl)amino]-1-(4′- hydroxy-2′-methyl-1,1′-biphenyl-3- yl)-1H-pyrazole-3-carboxamide | 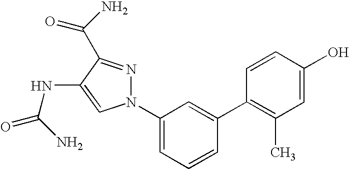 |
| 90 | 4-[(aminocarbonyl)amino]-1-{4′-[(4- oxopiperidin-1-yl)carbonyl]-1,1′- biphenyl-3-yl}-1H-pyrazole-3- carboxamide | 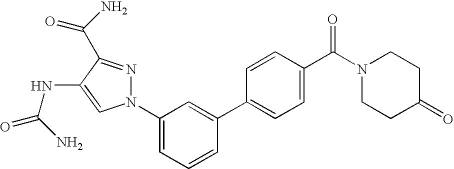 |
| 91 | 1-(2′-acetyl-1,1′-biphenyl-3-yl)-4- [(aminocarbonyl)amino]-1H- pyrazole-3-carboxamide | 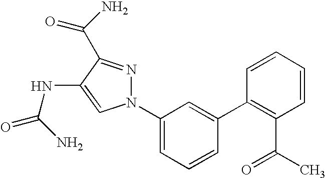 |
| 92 | 4-[(aminocarbonyl)amino]-1-(4′- fluoro-2′-hydroxy-1,1′-biphenyl-3- yl)-1H-pyrazole-3-carboxamide | 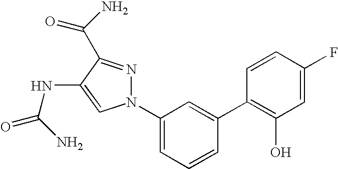 |
| 93 | 4-[(aminocarbonyl)amino]-1-(5′- fluoro-2′-hydroxy-1,1′-biphenyl-3- yl)-1H-pyrazole-3-carboxamide | 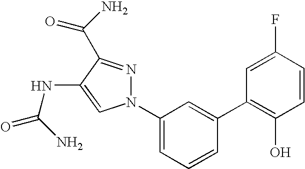 |
| 94 | 4-[(aminocarbonyl)amino]-1-[2′- (cyanomethoxy)-5′-fluoro-1,1′- biphenyl-3-yl]-1H-pyrazole-3- carboxamide | 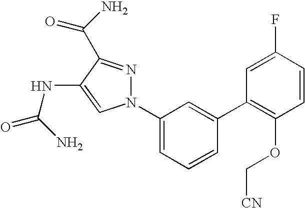 |
| 95 | 1-[4′-(aminocarbonyl)-1,1′-biphenyl- 4-yl]-4- {[(methylamino)carbonyl]amino}- 1H-pyrazole-3-carboxamide | 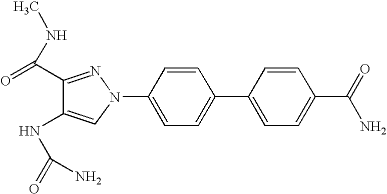 |
| 96 | 4′-{3-(aminocarbonyl)-4- [(aminocarbonyl)amino]-1H- pyrazol-1-yl}-1,1′-biphenyl-3- carboxylic acid | 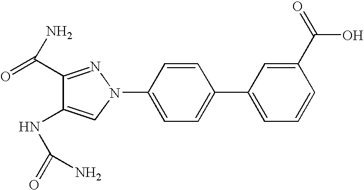 |
| 97 | 4′-{3-(aminocarbonyl)-4- [(aminocarbonyl)amino]-1H- pyrazol-1-yl}-3-fluoro-1,1′-biphenyl- 4-carboxylic acid | 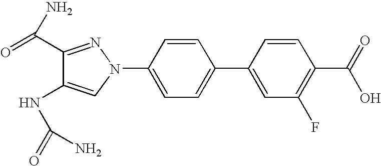 |
| 98 | 4-[(aminocarbonyl)amino]-1-(4′- fluoro-2′-hydroxy-1,1′-biphenyl-4- yl)-1H-pyrazole-3-carboxamide | 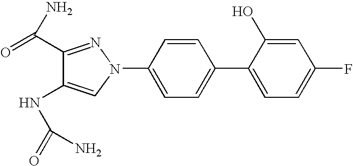 |
| 99 | 4-[(aminocarbonyl)amino]-1-(5′- fluoro-2′-hydroxy-1,1′-biphenyl-4- yl)-1H-pyrazole-3-carboxamide | 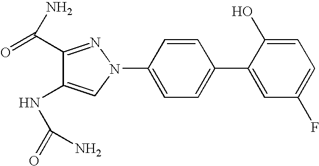 |
| 100 | 4-[(aminocarbonyl)amino]-1-(4′- cyano-1,1′-biphenyl-4-yl)-1H- pyrazole-3-carboxamide | 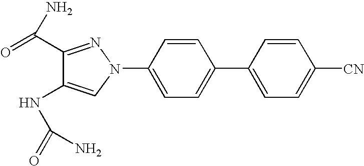 |
| 101 | 4-[(aminocarbonyl)amino]-1-(1,1′- biphenyl-4-yl)-1H-pyrazole-3- carboxamide | 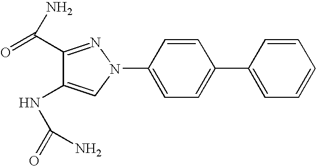 |
| 102 | 4-[(aminocarbonyl)amino]-1-(3′- methyl-1,1′-biphenyl-4-yl)-1H- pyrazole-3-carboxamide | 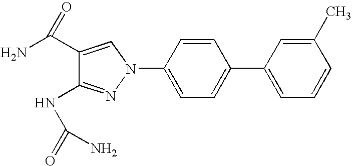 |
| 104 | 4-[(aminocarbonyl)amino]-1-(3′- cyano-1,1′-biphenyl-4-yl)-1H- pyrazole-3-carboxamide | 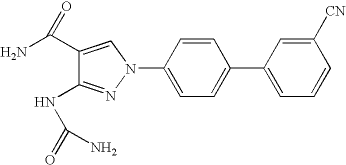 |
| 105 | 4-[(aminocarbonyl)amino]-1-[2′- (hydroxymethyl)-1,1′-biphenyl-4-yl]- 1H-pyrazole-3-carboxamide | 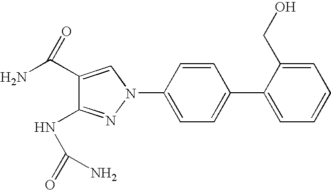 |
| 106 | 4-[(aminocarbonyl)amino]-1-(2′- methoxy-1,1′-biphenyl-4-yl)-1H- pyrazole-3-carboxamide | 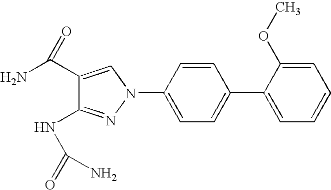 |
| 107 | 4-[(aminocarbonyl)amino]-1-(3′- methoxy-1,1′-biphenyl-4-yl)-1H- pyrazole-3-carboxamide | 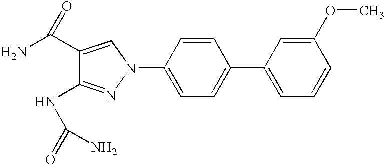 |
| 108 | 4-[(aminocarbonyl)amino]-1-(4′- fluoro-2′-methyl-1,1′-biphenyl-4-yl)- 1H-pyrazole-3-carboxamide | 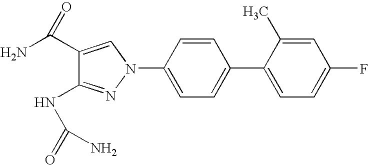 |
| 109 | 4-[(aminocarbonyl)amino]-1-(4′- fluoro-3′-methyl-1,1′-biphenyl-4-yl)- 1H-pyrazole-3-carboxamide | 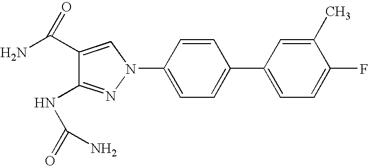 |
| 110 | 4-[(aminocarbonyl)amino]-1-(2′- chloro-1,1′-biphenyl-4-yl)-1H- pyrazole-3-carboxamide | 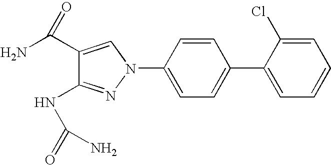 |
| 111 | 4-[(aminocarbonyl)amino]-1-(3′- chloro-1,1′-biphenyl-4-yl)-1H- pyrazole-3-carboxamide | 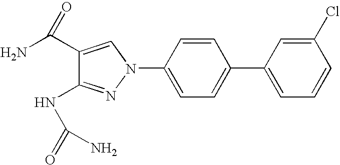 |
| 112 | 4-[(aminocarbonyl)amino]-1-(2′,3′- difluoro-1,1′-biphenyl-4-yl)-1- pyrazole-3-carboxamide | 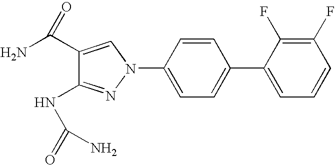 |
| 113 | 4-[(aminocarbonyl)amino]-1- difluoro-1,1′-biphenyl-4-yl)-1- pyrazole-3-carboxamide | 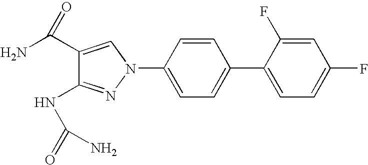 |
| 114 | 4-[(aminocarbonyl)amino]-1- difluoro-1,1′-biphenyl-4-yl)-1- pyrazole-3-carboxamide | 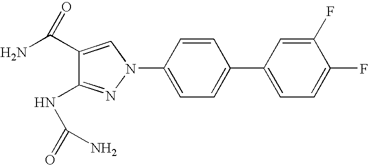 |
| 115 | 4-[(aminocarbonyl)amino]-1-(3′,5′- difluoro-1,1′-biphenyl-4-yl)-1H- pyrazole-3-carboxamide | 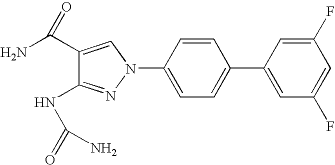 |
| 118 | 4-[(aminocarbonyl)amino]-1- (methylthio)-1,1′-biphenyl-4-yl]-1H- pyrazole-3-carboxamide | 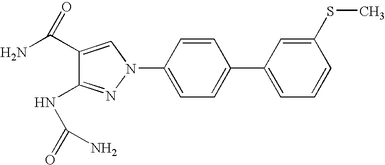 |
| 119 | 4-[(aminocarbonyl)amino]-1- (methylthio)-1,1′-biphenyl-4-yl]-1H- pyrazole-3-carboxamide | 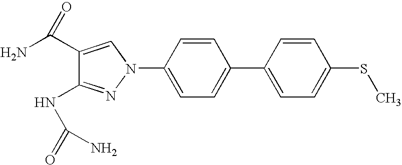 |
| 120 | 4-[(aminooarbonyl)amino]-1-(2′- fluoro-3′-methoxy-1,1′-biphenyl-4- yl)-1H-pyrazole-3-carboxamide | 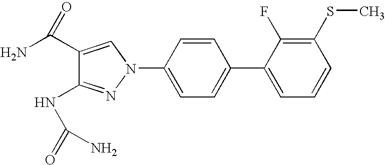 |
| 121 | 4-[(aminocarbonyl)amino]-1-(5′- fluoro-2′-methoxy-1,1′-biphenyl-4- yl)-1H-pyrazole-3-carboxamide | 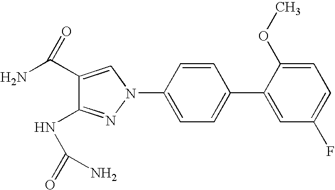 |
| 122 | 4-[(aminocarbonyl)amino]-1-(3′- fluoro-4′-methoxy-1,1′-biphenyl-4- yl)-1H-pyrazole-3-carboxamide | 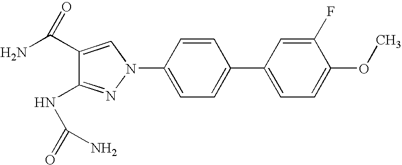 |
| 124 | 4-[(aminocarbonyl)amino]-1-[4-(1- naphthyl)phenyl]-1H-pyrazole-3- carboxamide | 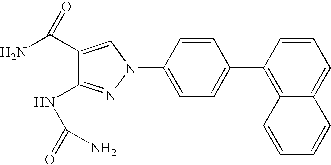 |
| 125 | 4-[(aminocarbonyl)amino]-1-(3′- chloro-4′-fluoro-1,1′-biphenyl-4-yl)- 1H-pyrazole-3-carboxamide | 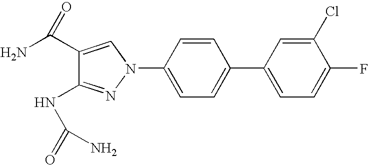 |
| 129 | 4-[(aminocarbonyl)amino]-1 -(5′- chloro-2′-methoxy-1,1′-biphenyl-4- yl)-1H-pyrazole-3-carboxamide | 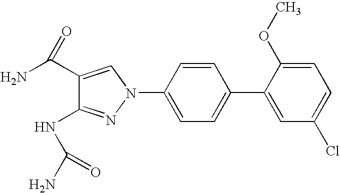 |
| 130 | 4-[(aminocarbonyl)amino]-1-[3′- (1H-pyrazol-1-yl)-1,1′-biphenyl-4- yl]-1H-pyrazole-3-carboxamide | 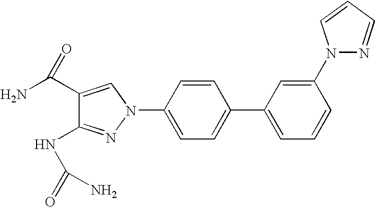 |
| 131 | 4-[(aminocarbonyl)amino]-1-[3′- (trifluoromethyl)-1,1′-biphenyl-4- 1H-pyrazole-3-carboxamide | 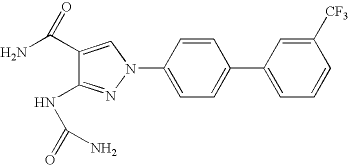 |
| 133 | 4-[(aminocarbonyl)amino]-1-(2′,3′- dichloro-1,1′-biphenyl-4-yl)- pyrazole-3-carboxamide | 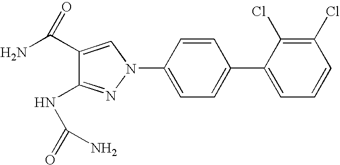 |
| 134 | 4-[(aminocarbonyl)amino]-1-(2′,4′- dichloro-1,1′-biphenyl-4-yl)-1- pyrazole-3-carboxamide | 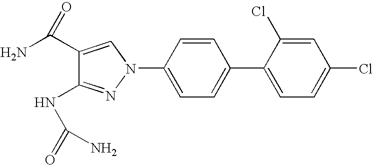 |
| 135 | 4-[(aminocarbonyl)amino]-1-(2′,5′- dichloro-1,1′-biphenyl-4-yl)-1- pyrazole-3-carboxamide | 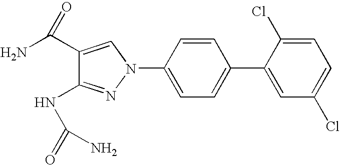 |
| 139 | 4-[(aminocarbonyl)amino]-1- (1,1′:3′,1″-terphenyl-4-yl)-1H- pyrazole-3-carboxamide | 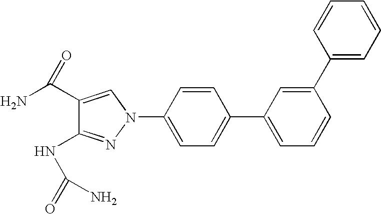 |
| 141 | 4-[(aminocarbonyl)amino]-1-[4-(1H- indol-2-yl)phenyl]-1H-pyrazole-3- carboxamide | 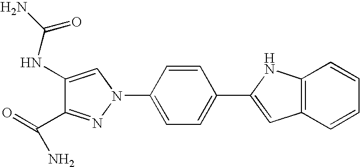 |
| 142 | 4′-{3-(aminocarbonyl)-4- [(aminocarbonyl)amino]-1H- pyrazol-1-yl}-1,1′-biphenyl-4- carboxylic acid | 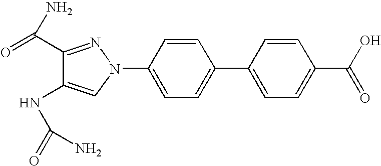 |
| 143 | 4-[(aminocarbonyl)amino]-1-{4′- [(diethylamino)carbonyl]-1- biphenyl-4-yl}-1H-pyrazole-3- carboxamide | 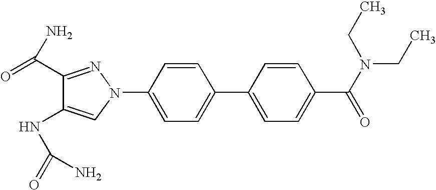 |
| 144 | ethyl 4′-{3-(aminocarbonyl)-4- [(aminocarbonyl)amino]-1H- pyrazol-1-yl}-1,1′-biphenyl-3- carboxylate | 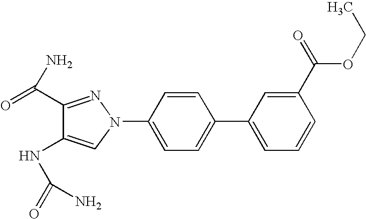 |
| 145 | 4-[(aminocarbonyl)amino]-1-{4′- [(methylamino)carbonyl]-1,1′- biphenyl-4-yl}-1H-pyrazole-3- carboxamide | 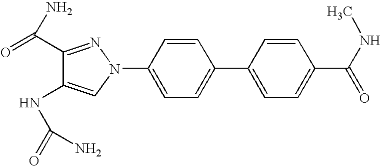 |
| 146 | 4-[(aminocarbonyl)amino]-1-{4′- [(propylamino)carbonyl]-1,1′- biphenyl-4-yl}-1H-pyrazole-3- carboxamide | 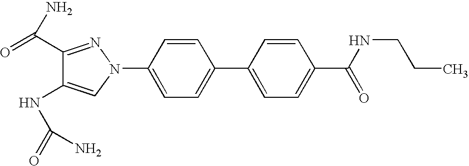 |
| 147 | ethyl 4′-{3-(aminocarbonyl)-4- [(aminocarbonyl)amino]-1H- pyrazol-1-yl}-1,1′-biphenyl-4- carboxylate | 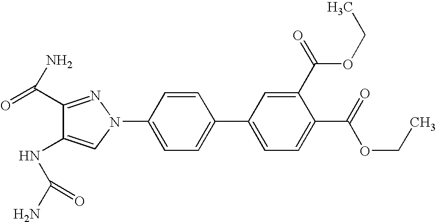 |
| 148 | 4-[(aminacarbonyl)amina]-1-{3′- [(isopropylamino)carbonyl]-1,1′- biphenyl-4-yl}-1H-pyrazole-3- carboxamide | 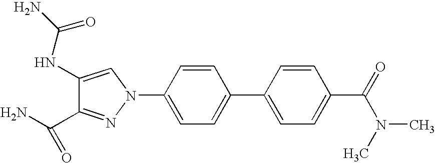 |
| 149 | 4-[(aminocarbonyl)amino]-1-[4′- (aminocarbonyl)-1,1′-biphenyl-4-yl]- 1H-pyrazole-3-carboxamide | 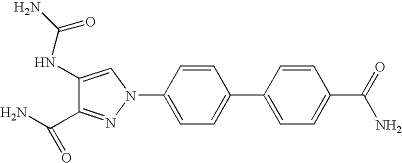 |
| 150 | 4-[(aminocarbonyl)amino]-1-(3′- fluoro-4′-{[(4- methoxybenzyl)amino]carbonyl}- 1,1′-biphenyl-4-yl)-1H-pyrazole-3- carboxamide | 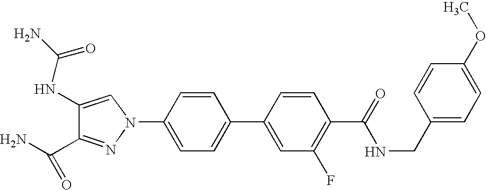 |
| 151 | 4-[(aminocarbonyl)amino]-1-{4′- [(benzylamino)carbonyl]-3′- 1,1′-biphenyl-4-yl}-1H- carboxamide | 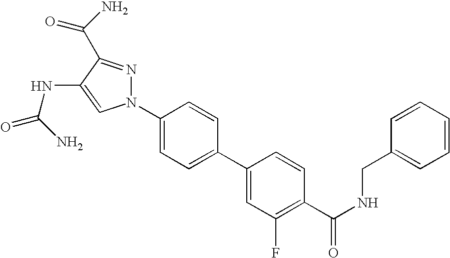 |
| 152 | 4-[(aminocarbonyl)amino]-1-{3′- fluoro-4′-[(methylamino)carbonyl]- 1,1′-biphenyl-4-yl}-1H-pyrazole-3- carboxamide | 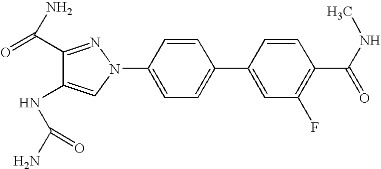 |
| 153 | 4-[(aminocarbonyl)amino]-1-(3′- fluoro-4′-{[(3- methoxybenzyl)amino]carbonyl}- 1,1′-biphenyl-4-yl)-1H-pyrazole-3- carboxamide | 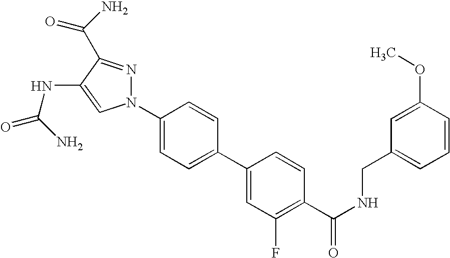 |
| 154 | 4-[(aminocarbonyl)amino]-1-(3′- fluoro-4′-{[(2- methoxybenzyl)amino]carbonyl}- 1,1′-biphenyl-4-yl)-1H-pyrazole-3- carboxamide | 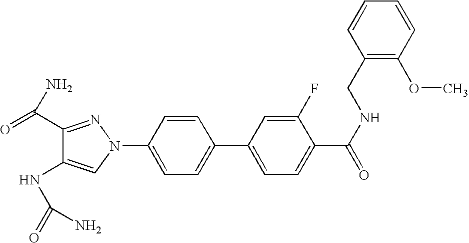 |
| 155 | 4-[(aminocarbonyl)amino]-1-(3′- fluoro-4′-{[(4- fluorobenzyl)amino]carbonyl}-1,1′- biphenyl-4-yl)-1H-pyrazole-3- carboxamide | 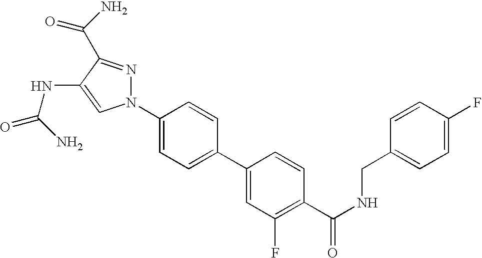 |
| 156 | 4-[(aminocarbonyl)amino]-1-(3′- fluoro-4′-{[(3- fluorobenzyl)amino]carbonyl}-1,1′- biphenyl-4-yl-1H-pyrazole-3- carboxamide | 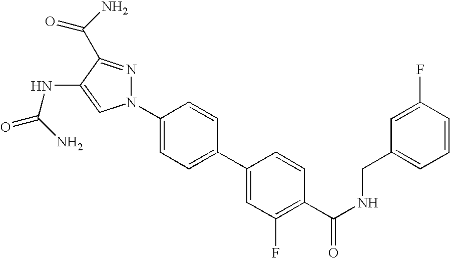 |
| 157 | 4-[(aminocarbonyl)amino]-1-(3′- fluoro-4′-{[(2- fluorobenzyl)amino]carbonyl}-1,1′- biphenyl-4-yl)-1H-pyrazole-3- carboxamide | 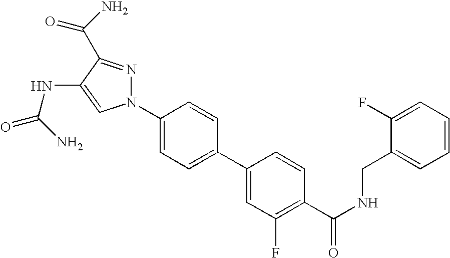 |
| 158 | 4-[(aminocarbonyl)amino]-1-(3′- fluoro-4′-{[(4- fluorobenzyl)(methyl)amino]carbon yl}-1,1′-biphenyl-4-yl)-1H-pyrazole- 3-carboxamide | 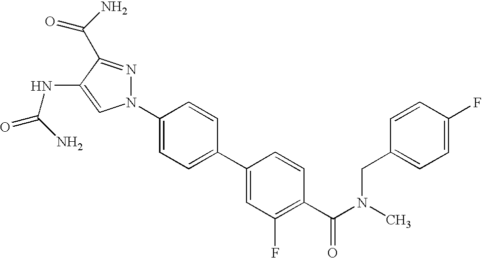 |
| 159 | 4-[(aminocarbonyl)amino]-1-(3′- fluoro-4′-[(1,2,3,4- tetrahydronaphthalen-1- ylamino)carbonyl]-1,1′-biphenyl-4- yl}-1H-pyrazole-3-carboxamide | 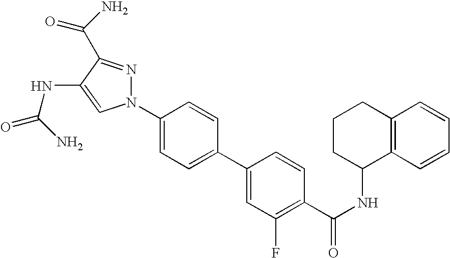 |
| 160 | 4-[(aminocarbonyl)amino]-1-(4′- {[[2- (dimethylamino)ethyl](methyl)amino]carbonyl}-3′-fluoro-1,1′-biphenyl- 4-yl)-1H-pyrazole-3-carboxamide | 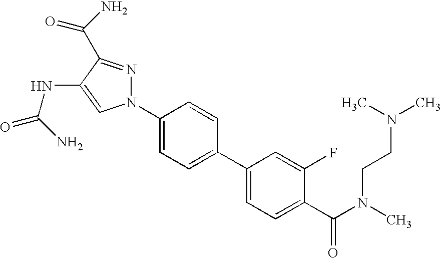 |
| 161 | 4-[(aminocarbonyl)amino]-1-[5′- fluoro-2′-(2-morpholin-4-yl-2- oxoethoxy)-1,1′-biphenyl-4-yl]-1H- pyrazole-3-carboxamide | 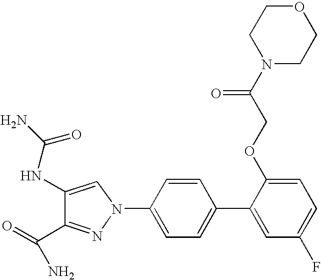 |
| 164 | 4-fluorobenzyl 4′-{3- (aminocarbonyl)-4- [(aminocarbonyl)amino]-1H- pyrazol-1-yl}-3-fluoro-1,1′-biphenyl- 4-carboxylate | 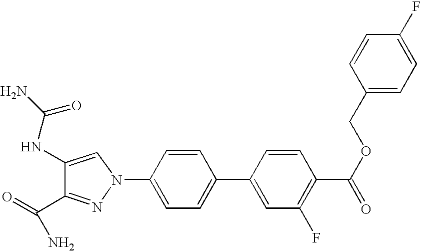 |
| 165 | 4′-{3-(aminocarbonyl)-4- [(aminocarbonyl)amino]-1H- pyrazol-1-yl}-1,1′-biphenyl-3- yl)methyl acetate | 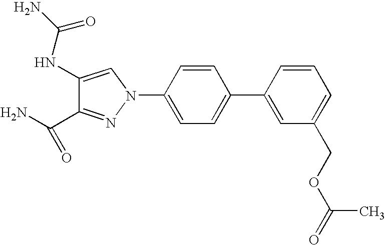 |
| 166 | 4-[(aminocarbonyl)amino]-1-[3′- (cyanomethoxy)-1,1′-biphenyl-4-yl]- 1H-pyrazole-3-carboxamide | 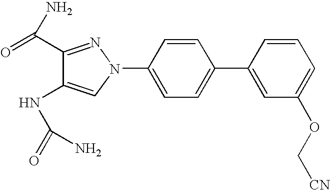 |
| 167 | 4-[(aminocarbonyl)amino]-1-[2′- (cyanomethoxy)-1,1′-biphenyl-4-yl]- 1H-pyrazole-3-carboxamide | 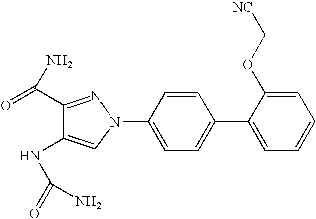 |
| 168 | 4-[(aminocarbonyl)amino]-1-[3′- (pyridin-2-ylmethoxy)-1,1′-biphenyl- 4-yl]-1H-pyrazole-3-carboxamide | 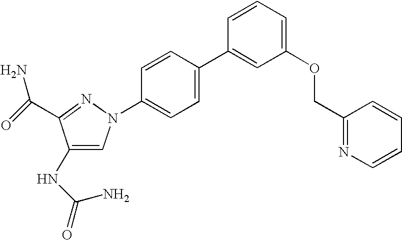 |
| 169 | 4-[(aminocarbonyl)amino]-1-[3′-(1- cyanoethoxy)-1,1′-biphenyl-4-yl]- 1H-pyrazole-3-carboxamide | 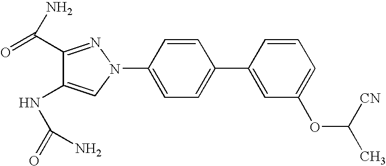 |
| 170 | 4-[(aminocarbonyl)amino]-1-[3′- (pyridin-3-ylmethoxy)-1,1′-biphenyl- 4-yl]-1H-pyrazole-3-carboxamide | 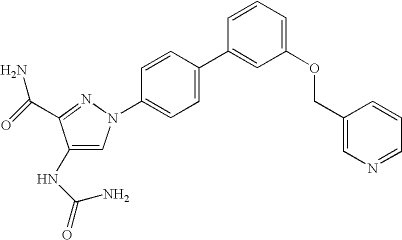 |
| 171 | 4-[(aminocarbonyl)amino]-1-[3′- (pyridin-4-ylmethoxy)-1,1′-biphenyl- 4-yl]-1H-pyrazole-3-carboxamide | 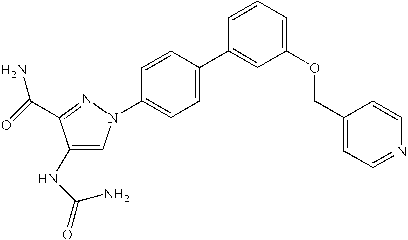 |
| 172 | 4-[(aminocarbonyl)amino]-1-[2′- (cyanomethoxy)-5′-fluoro-1,1′- biphenyl-4-yl]-1H-pyrazole-3- carboxamide | 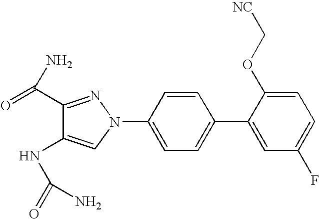 |
| 173 | 4-[(aminocarbonyl)amino]-1-[2′- (cyanomethoxy)-4′-fluoro-1,1′- biphenyl-4-yl]-1H-pyrazole-3- carboxamide | 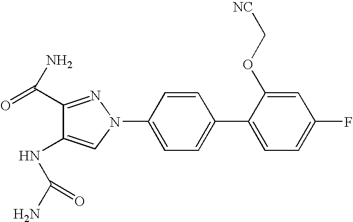 |
| 174 | tert-butyl [(4′-{3-(aminocarbonyl)-4- [(aminocarbonyl)amino]-1H- pyrazol-1-yl}-1,1′-biphenyl-3- yl)oxy]acetate | 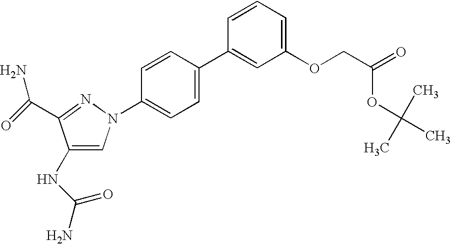 |
| 175 | 4-[(aminocarbonyl)amino]-1-[3′-(2- amino-2-oxoethoxy)-1,1′-biphenyl- 4-yl]-1H-pyrazole-3-carboxamide | 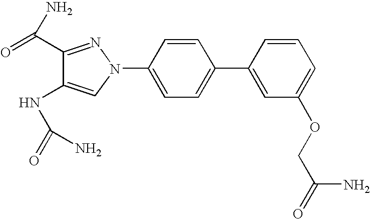 |
| 176 | 4-[(aminocarbonyl)amino]-1-[2′- (cyanomethoxy)-1,1′-biphenyl-3-yl]- 1H-pyrazole-3-carboxamide | 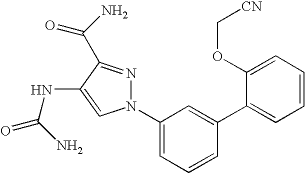 |
| 177 | 4-[(aminocarbonyl)amino]-1-[4′- (oyanomethoxy)-1,1′-biphenyl-3-yl]- 1H-pyrazole-3-carboxamide | 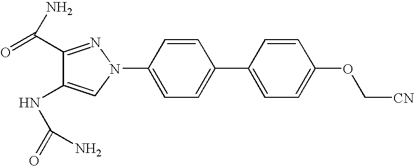 |
| 178 | 4-[(aminocarbonyl)amino]-1-[3′- (cyanomethoxy)-1,1′-biphenyl-3-yl]- 1H-pyrazole-3-oarboxamide | 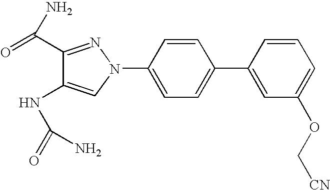 |
| 179 | 4-[(aminocarbonyl)amino]-1-[3′- (cyanomethoxy)-3-fluoro-1,1′- biphenyl-4-yl]-1H-pyrazole-3- carboxamide | 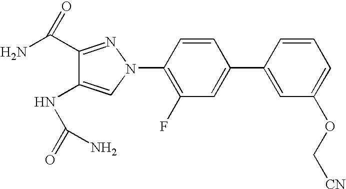 |
| 180 | 4-[(aminocarbonyl)amino]-1-[2′-(1- cyanoethoxy)-5′-fluoro-1,1′- biphenyl-4-yl]-1H-pyrazole-3- carboxamide | 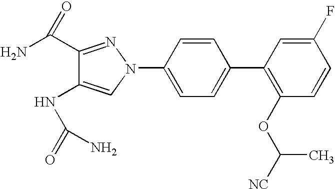 |
| 181 | 4-[(aminocarbonyl)amino]-1-{3′-[2- (dimethylamino)-2-oxoethoxy]-1,1′- biphenyl-4-yl}-1H-pyrazole-3- carboxamide | 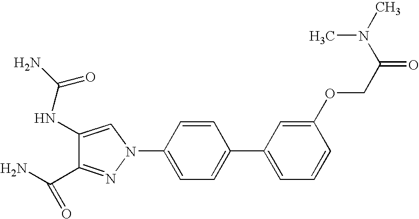 |
| 183 | 4-[(aminocarbonyl)amino]-1-[4′- (aminocarbonyl)-3′-fluoro-1,1′- biphenyl-4-yl]-1H-pyrazole-3- carboxamide | 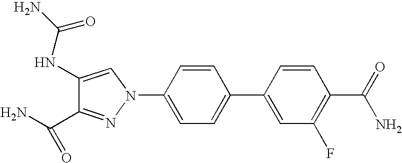 |
| 184 | methyl 4-{3-(aminocarbonyl)-4- [(aminocarbonyl)amino]-1H- pyrazol-1-yl}benzoate | 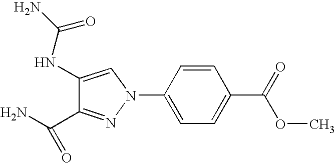 |
| 185 | methyl 3-{3-(aminocarbonyl)-4- [(aminocarbonyl)amino]-1H- pyrazol-1-yl}benzoate | 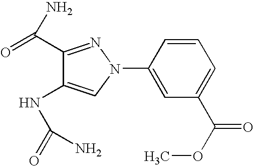 |
| 186 | 4-[(aminocarbonyl)amino]-1-[3′-(2- cyanoethyl)-1,1′-biphenyl-4-yl]-1H- pyrazole-3-carboxamide | 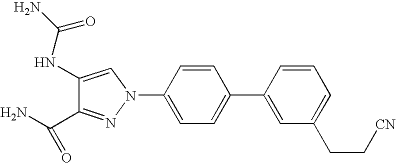 |
| 187 | 4-[(aminocarbonyl)amino]-1-[4′- (1H-tetrazol-5-yl)-1,1′-biphenyl-4- yl]-1H-pyrazole-3-carboxamide | 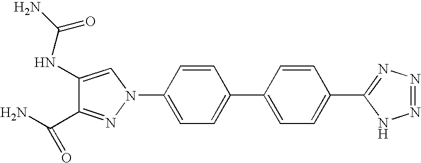 |
| 188 | 4-[(aminocarbonyl)amino]-1-(3,4- dichlorophenyl)-1H-pyrazole-3- carboxamide | 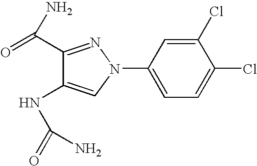 |
| 189 | 1-(3-carbamoyl-1-(4- (cyclohexylthio)phenyl)-1H-pyrazol- 4-yl)urea | 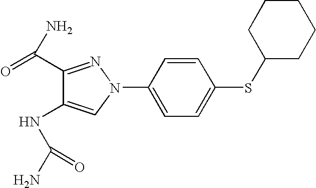 |
| 190 | 1-(1-(4-(2-fluorophenylthio)phenyl)- 3-carbamoyl-1H-pyrazol-4-yl)urea | 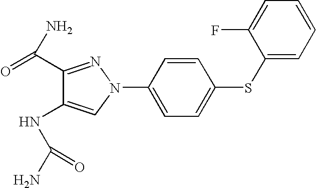 |
| 191 | 1-(3-carbamoyl-1-(4- (cyclohexylsulfinyl)phenyl)-1H- pyrazol-4-yl)urea | 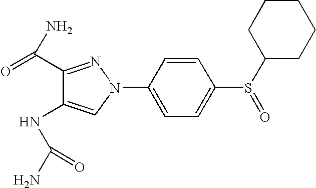 |
| 192 | 1-(3-carbamoyl-1-(4- (cyclohexylsulfonyl)phenyl)-1H- pyrazol-4-yl)urea | 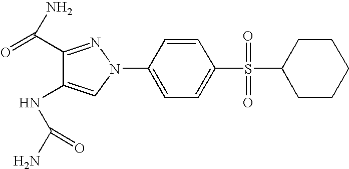 |
| 193 | 1-(1-(4-(2- fluorophenylsulfinyl)phenyl)-3- carbamoyl-1H-pyrazol-4-yl)urea | 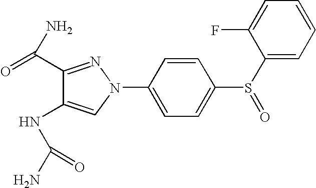 |
| 194 | 1-(1-(4-(2- fluorophenylsulfonyl)phenyl)-3- carbamoyl-1H-pyrazol-4-yl)urea | 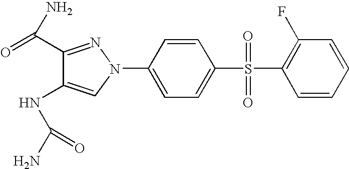 |
| 195 | 1-(3-carbamoyl-1-(3-iodophenyl)- 1H-pyrazol-4-yl)urea | 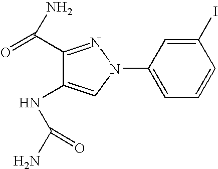 |
| 196 | 1-(1-(4-(4-fluorophenylthio)phenyl)- 3-carbamoyl-1H-pyrazol-4-yl)urea | 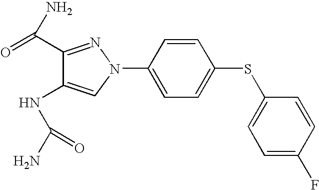 |
| 197 | 1-(1-(4-(3-fluorophenylthio)phenyl)- 3-carbamoyl-1H-pyrazol-4-yl)urea | 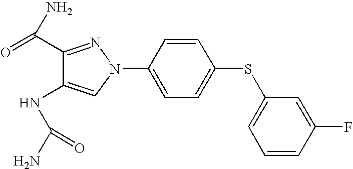 |
| 198 | 1-(1-(3-(2-fluorophenylthio)phenyl)- 3-carbamoyl-1H-pyrazol-4-yl)urea | 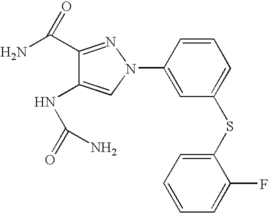 |
| 199 | 1-(1-(4-(4- hydroxyphenylthio)phenyl)-3- carbamoyl-1H-pyrazol-4-yl)urea | 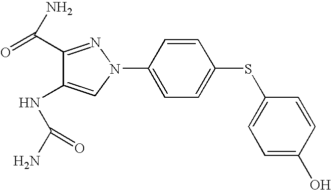 |
| 200 | 1-(1-(4-(4- fluorophenylsulfinyl)phenyl)-3- carbamoyl-1H-pyrazol-4-yl)urea | 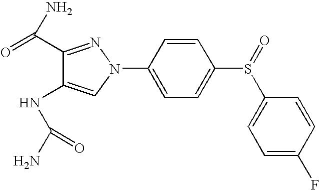 |
| 201 | 1-(1-(4-(3- fluorophenylsulfinyl)phenyl)-3- carbamoyl-1H-pyrazol-4-yl)urea | 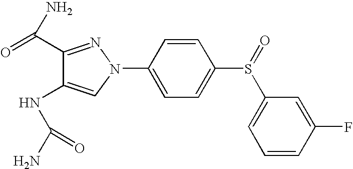 |
| 202 | 1-(1-(3-(2- fluorophenylsulfinyl)phenyl)-3- carbamoyl-1H-pyrazol-4-yl)urea | 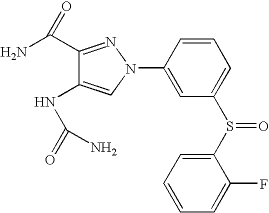 |
| 203 | 1-(1-(4-(3- fluorophenylsulfonyl)phenyl)-3- carbamoyl-1H-pyrazol-4-yl)urea | 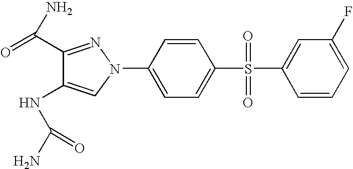 |
| 204 | 1-(1-(4-(4- fluorophenylsulfonyl)phenyl)-3- carbamoyl-1H-pyrazol-4-yl)urea | 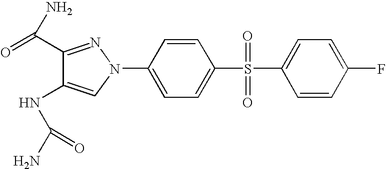 |
| 205 | 1-(3-carbamoyl-1-(4-(pyridin-2- ylthio)phenyl)-1H-pyrazol-4-yl)urea | 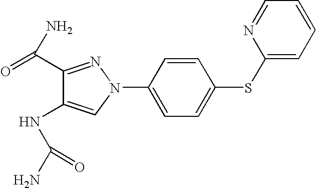 |
| 206 | 1-(3-carbamoyl-1-(4-(pyridin-4- ylthio)phenyl)-1H-pyrazol-4-yl)urea | 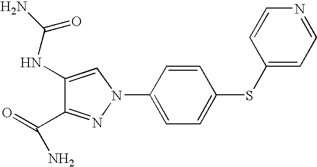 |
| 207 | 1-(3-carbamoyl-1-(4- (cyclopentyhhio)phenyl)-1H- pyrazol-4-yl)urea | 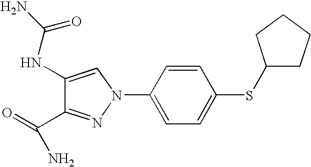 |
| 208 | 1-(1-(4-(2- hydroxyphenylthio)phenyl)-3- carbamoyl-1H-pyrazol-4-yl)urea | 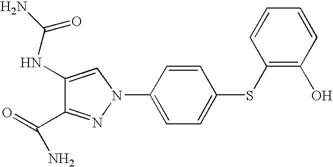 |
| 209 | 4-[(Aminocarbonyl)amino]-1-(3- chloro-4-methoxyphenyl)-1H- pyrazole-3-carboxamide | 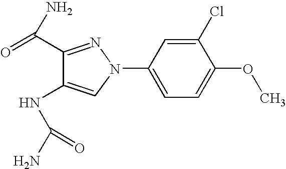 |
| 210 | 1-(1-(4-(2-fluorophenylthio)-2- fluorophenyl)-3-carbamoyl-1H- pyrazol-4-yl)urea | 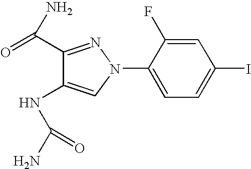 |
| 212 | 4-[(aminocarbonyl)amino]-1-(4- fluorophenyl)-1H-pyrazole-3- carboxamide | 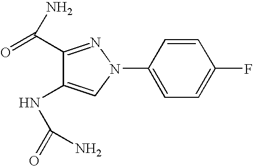 |
| 213 | 4-[(aminocarbonyl)amino]-1-(1,3- benzodioxol-5-yl)-1H-pyrazole-3- carboxamide | 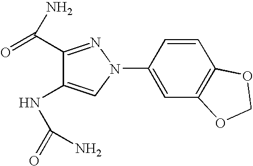 |
| 214 | 4-[(aminocarbonyl)amino]-1-(4- chlorophenyl)-1H-pyrazole-3- carboxamide | 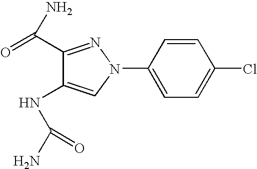 |
| 215 | 4-[(aminocarbonyl)amino]-1-(4- iodophenyl)-1H-pyrazole-3- carboxamide | 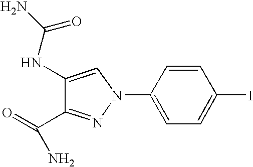 |
| 216 | 4-[(aminooarbonyl)amino]-1-(4- isopropylphenyl)-1H-pyrazole-3- carboxamide | 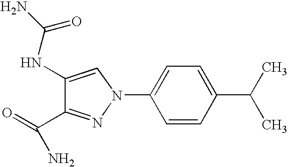 |
| 217 | 4-[(aminocarbonyl)amino]-1-(1H- indol-5-yl)-1H-pyrazole-3- carboxamide | 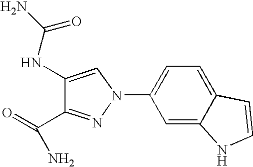 |
| 218 | 4-[(aminocarbonyl)amino]-1-(3,4- difluorophenyl)-1H-pyrazole-3- carboxamide | 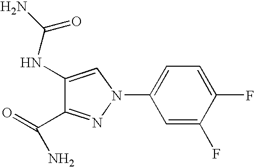 |
| 219 | 4-[(aminocarbonyl)amino]-1-(2- naphthyl)-1H-pyrazole-3- carboxamide | 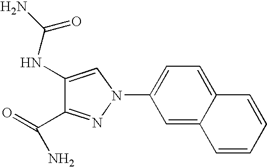 |
| 220 | 4-[(aminocarbonyl)amino]-1-[2- chloro-3′-(cyanomethoxy)-1,1′- biphenyl-4-yl]-1H-pyrazole-3- carboxamide | 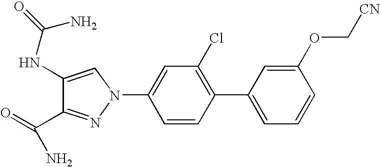 |
| 221 | 4-[(aminocarbonyl)amino]-1-{3- chloro-4-[(4- fluorophenyl)thio]phenyl}-1H- pyrazole-3-carboxamide | 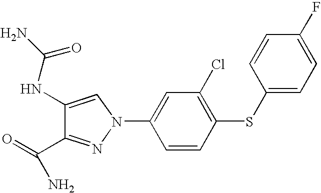 |
| 223 | 4-[(aminocarbonyl)amino]-1-(2- chloro-2′-hydroxy-1,1′-biphenyl-4- yl)-1H-pyrazole-3-carboxamide | 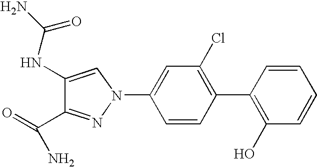 |
| 224 | 4-[(aminocarbonyl)amino]-1-[2- chloro-2′-(cyanomethoxy)-1,1′- biphenyl-4-yl]-1H-pyrazole-3- carboxamide | 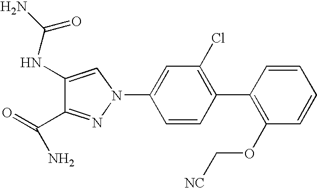 |
| 225 | 4-[(aminocarbonyl)amino]-1-(4- bromo-3-fluorophenyl)-1H- pyrazole-3-carboxamide | 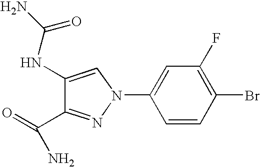 |
| 226 | 4-[(aminocarbonyl)amino]-1-(2- fluoro-3′-hydroxy-1,1′-biphenyl-4- yl)-1H-pyrazole-3-carboxamide | 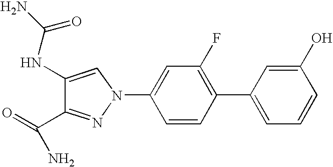 |
| 227 | 4-[(aminocarbonyl)amino]-1-[3′- (cyanomethoxy)-2-fluoro-1,1′- biphenyl-4-yl]-1H-pyrazole-3- carboxamide | 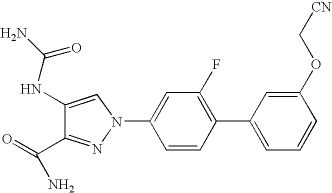 |
| 229 | 4-[(aminocarbonyl)amino]-1-{4-[(4- fluorophenylsulfonyl]-2- methylphenyl}-1H-pyrazole-3- carboxamide | 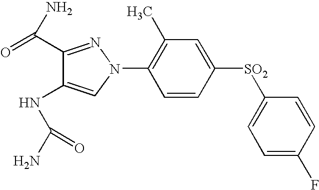 |
| 230 | 4-[(aminocarbonyl)amino]-1-(2- ethoxy-3′-hydroxy-1,1′-biphenyl-4- yl)-1H-pyrazole-3-carboxamide | 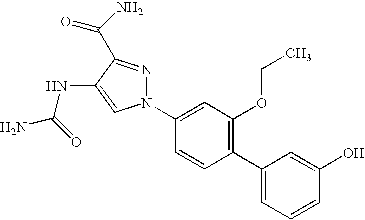 |
| 231 | 4-[(aminocarbonyl)amino]-1-[3′- (cyanomethoxy)-2-ethoxy-1,1′- biphenyl-4-yl]-1H-pyrazole-3- carboxamide | 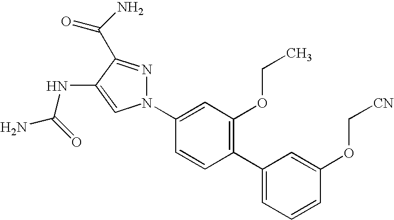 |
| 233 | 4-[(aminooarbonyl)amino]-1-[3′- hydroxy-2-(trifluoromethyl)-1,1′- biphenyl-4-yl]-1H-pyrazole-3- carboxamide | 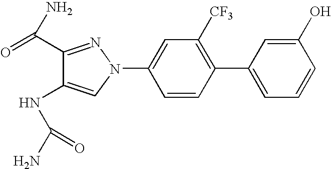 |
| 234 | 4-[(aminocarbonyl)amino]-1-[2′- hydroxy-2-(trifluoromethyl)-1,1′- biphenyl-4-yl]-1H-pyrazole-3- carboxamide | 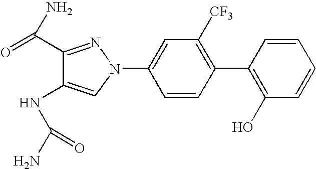 |
| 236 | 4-[(aminocarbonyl)amino]-1-[2′- (cyanomethoxy)-2-ethoxy-1,1′- biphenyl-4-yl]-1H-pyrazole-3- carboxamide | 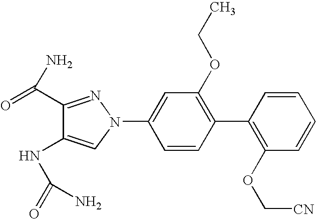 |
| 237 | 4-[(aminocarbonyl)amino]-1-[3′- (cyanomethoxy)-2-(trifluoromethyl)- 1,1′-biphenyl-4-yl]-1H-pyrazole-3- carboxamide | 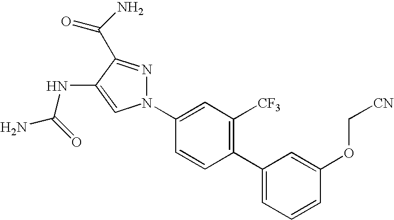 |
| 238 | 4-[(aminocarbonyl)amino]-1-[2′- (cyanomethoxy)-2-(trifluoromethyl)- 1,1′-biphenyl-4-yl]-1H-pyrazole-3- carboxamide | 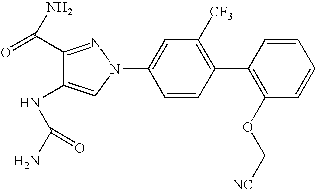 |
| 239 | 1-(3′-hydroxy-2-methyl-biphenyl-4- yl)-4-ureido-1H-pyrazole-3- carboxylic acid amide | 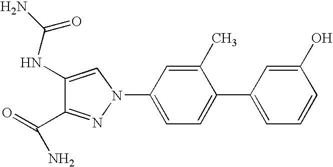 |
| 240 | 1-(2′-Hydroxy-2-methyl-biphenyl-4- yl)-4-ureido-1H-pyrazole-3- carboxylic acid amide | 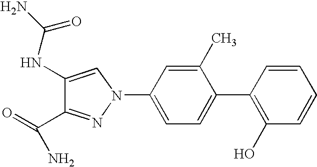 |
| 241 | 1-(4′-Fluoro-2′-hydroxy-2-methyl- biphenyl-4-yl)-4-ureido-1H- pyrazole-3-carboxylic acid amide | 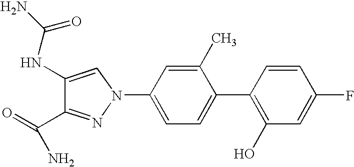 |
| 242 | 1-(5′-Fluoro-2′-hydroxy-2-methyl- biphenyl-4-yl)-4-ureido-1H- pyrazole-3-carboxylic acid amide | 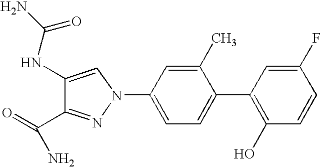 |
| 243 | 1-(3′-Cyanomethoxy-2-methyl- biphenyl-4-yl)-4-ureido-1H- pyrazole-3-carboxylic acid amide | 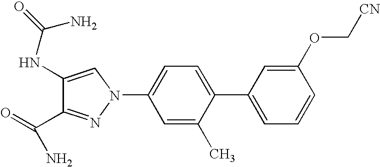 |
| 244 | 1-(2′-Cyanomethoxy-4′-fluoro-2- methyl-biphenyl-4-yl)-4-ureido-1H- pyrazole-3-carboxylic acid amide | 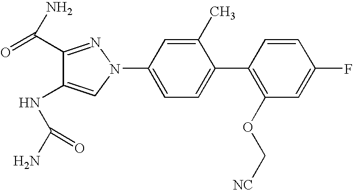 |
| 245 | 1-(4-Iodo-3-methyl-phenyl)-4-(3- methyl-ureido)-1H-pyrazole-3- carboxylic acid amide | 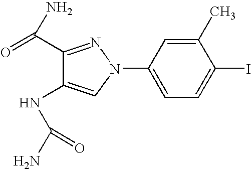 |
| 246 | 1-(5-chloro-2-fluoro-phenyl)-4- ureido-1H-pyrazole-3-carboxylic acid amide | 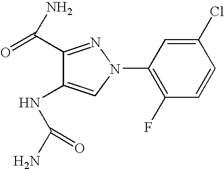 |
| 247 | 4-[(aminocarbonyl)amino]-1-(3- fluoro-3′-hydroxy-1,1′-biphenyl-4- yl)-1H-pyrazole-3-carboxamide | 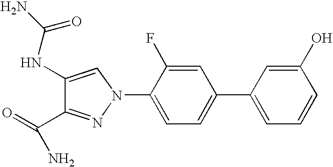 |
| 248 | 4′-{3-(aminocarbonyl)-4- [(aminocarbonyl)amino]-1H- pyrazol-1-yl}-3,3′-difluoro-1,1′- biphenyl-4-carboxylic acid | 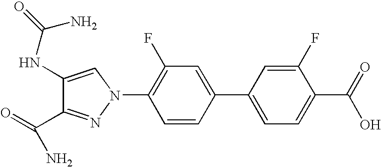 |
| 249 | 4-[(aminocarbonyl)amino]-1-(3- fluoro-2′-hydroxy-1,1′-biphenyl-4- yl)-1H-pyrazole-3-carboxamide | 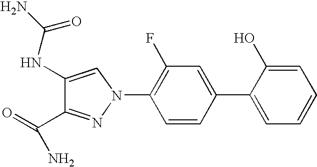 |
| 250 | 4-[(aminocarbonyl)amino]-1-[2′- (cyanomethoxy)-3-fluoro-1,1′- biphenyl-4-yl]-1H-pyrazole-3- carboxamide | 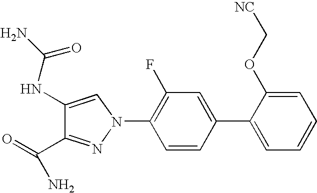 |
| 251 | 4-[(aminocarbonyl)aminol-1-(2- chloro-4-iodophenyl)-1H-pyrazole- 3-carboxamide | 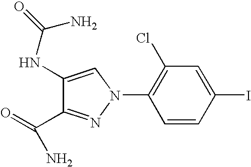 |
| 252 | 4-[(aminocarbonyl)amino]-1-[2′-(2- amino-2-oxoethoxy)-5′-fluoro-1,1- biphenyl-4-yl]-1H-pyrazole-3- carboxamide | 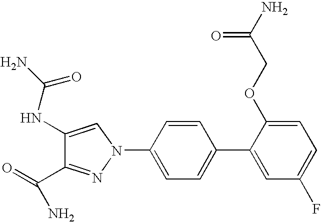 |
| 253 | 4-[(aminocarbonyl)amino]-1-[3′-(2- oxopropoxy)-1,1′-biphenyl-4-yl]-1H- pyrazole-3-carboxamide | 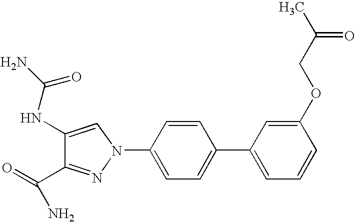 |
| 254 | 4-[(aminocarbonyl)amino]-1-[3′- (1,3-oxazol-2-ylmethoxy)-1,1′- biphenyl-4-yl]-1H-pyrazole-3- carboxamide | 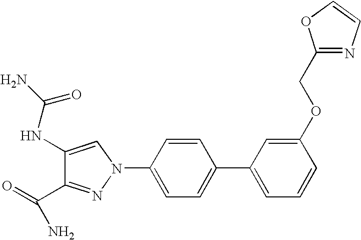 |
| 255 | 4-[(aminocarbonyl)amino]-1-[5′- fluoro-2′-(1,3-oxazol-2-ylmethoxy)- 1,1′-biphenyl-4-yl]-1H-pyrazole-3- carboxamide | 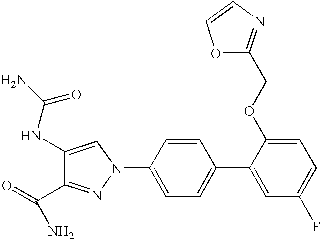 |
| 256 | 4-[(aminocarbonyl)amino]-1-{5′- fluoro-2′-[2-(methylamino)-2- oxoethoxy]-1,1′-biphenyl-4-yl}-1H- pyrazole-3-carboxamide | 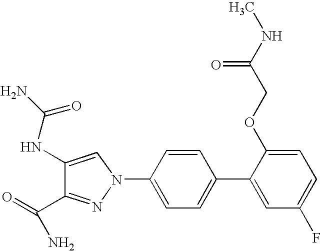 |
| 257 | 4-[(aminocarbonyl)amino]-1-{5′- fluoro-2′-[2-(ethylamino)-2- oxoethoxy]-1,1′-biphenyl-4-yl}-1H- pyrazole-3-carboxamide | 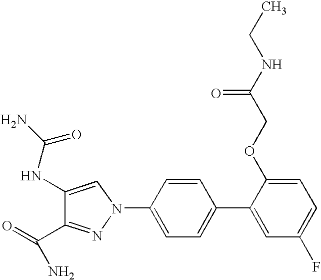 |
| 258 | [(4′-{3-(aminocarbonyl)-4- [(aminocarbonyl)aminol-1H- pyrazol-1-yl}-1,1′-biphenyl-3- yl)oxy]acetic acid | 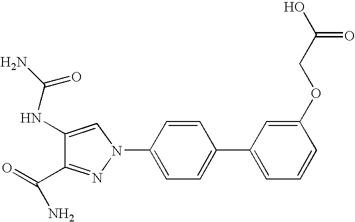 |
| 259 | [(4′-{3-(aminocarbonyl)-4- [(aminocarbonyl)amino]-1H- pyrazol-1-yl}-5-fluoro-1,1′-biphenyl- 2-yl)oxy]acetic acid | 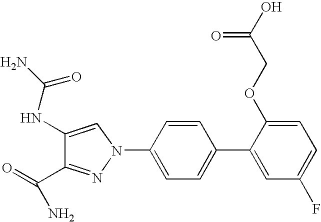 |
| 260 | 4-[(aminocarbonyl)amino]-1-[4- (4,4,5,5-tetramethyl-1,3,2- dioxaborolan-2-yl)phenyl]-1H- pyrazole-3-carboxamide | 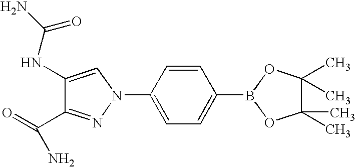 |
| 261 | 4-[(aminocarbonyl)amino]-1-(2- fluoro-2′-hydroxy-1,1′-biphenyl-4- yl)-1H-pyrazole-3-carboxamide | 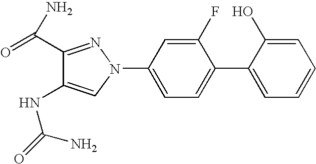 |
| 262 | 4-[(aminocarbonyl)amino]-1-(2,4′- difluoro-2′-hydroxy-1,1′-biphenyl-4- yl)-1H-pyrazole-3-carboxamide | 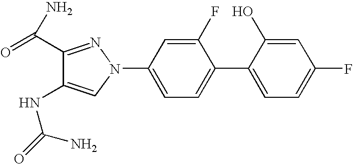 |
| 263 | 4-[(aminocarbonyl)amino]-1-(2,5′- difluoro-2′-hydroxy-1,1′-biphenyl-4- yl)-1H-pyrazole-3-carboxamide | 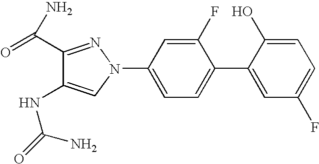 |
| 264 | 4-[(aminocarbonyl)amino]-1-(2- fluoro-4′-hydroxy-1,1′-biphenyl-4- yl)-1H-pyrazole-3-carboxamide | 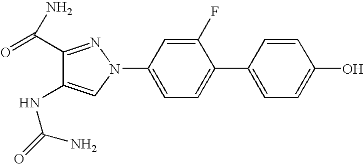 |
| 265 | 4-[(aminocarbonyl)amino]-1-(2,3′- difluoro-4′-hydroxy-1,1′-biphenyl-4- yl)-1H-pyrazole-3-carboxamide | 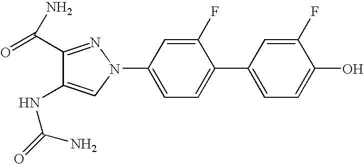 |
| 266 | 4-[(aminocarbonyl)amino]-1-[2′-(2- amino-2-oxoethoxy)-2-fluoro-1,1′- biphenyl-4-yl]-1H-pyrazole-3- carboxamide | 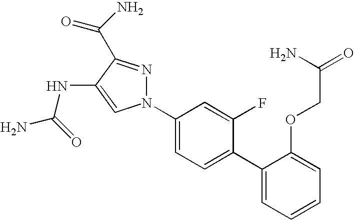 |
| 267 | 4-[(aminocarbonyl)amino]-1-(2- fluoro-2′-methoxy-1,1′-biphenyl-4- yl)-1H-pyrazole-3-carboxamide | 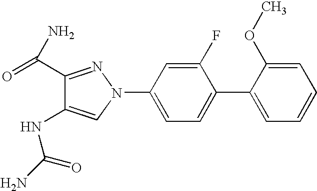 |
| 268 | 4-[(aminocarbonyl)amino]-1-[2′-(2- amino-2-oxoethoxy)-2,4′-difluoro- 1,1′-biphenyl-4-yl]-1H-pyrazole-3- carboxamide | 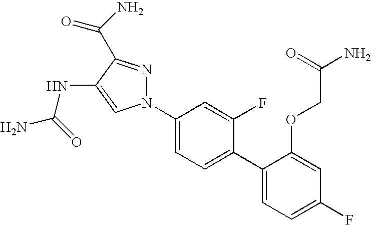 |
| 269 | 4-[(aminocarbonyl)amino]-1-(2,5′- difluoro-2′-methoxy-1,1′-biphenyl-4- yl)-1H-pyrazole-3-carboxamide | 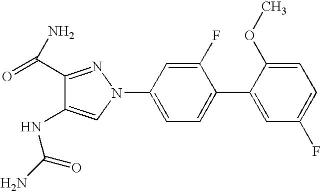 |
| 270 | 4-[(aminocarbonyl)amino]-1-{3- chloro-4-[(2- fluorophenyl)thio]phenyl}-1H- pyrazole-3-carboxamide | 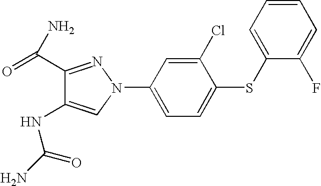 |
| 271 | 4-[(aminocarbonyl)amino]-1-{3- chloro-4-[(2- fluorophenyl)sulfonyl]phenyl}-1H- pyrazole-3-carboxamide | 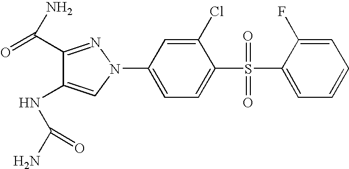 |
| 272 | 4-[(aminocarbonyl)amino]-1-(2- chloro-4′-hydroxy-1,1′-biphenyl-4- yl)-1H-pyrazole-3-carboxamide | 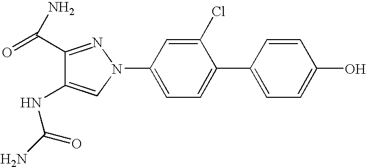 |
| 273 | 1-(3-chloro-2-fluoro-phenyl)-4- ureido-1h-pyrazole-3-carboxylic acid amide | 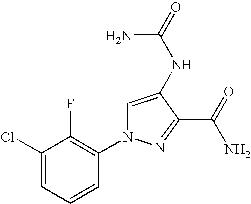 |
| 276 | 4-[(aminocarbonyl)amino]-1-(4- bromo-3-hydroxyphenyl)-1H- pyrazole-3-carboxamide | 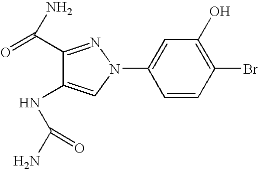 |
| 277 | (2E)-2-[(4-bromo-2- ethylphenyl)hydrazono]-2- cyanoacetamide | 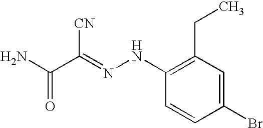 |
| 283 | 1-(3-carbamoyl-1-(4- (cyclopentylsulfinyl)phenyl)-1H- pyrazol-4-yl)urea | 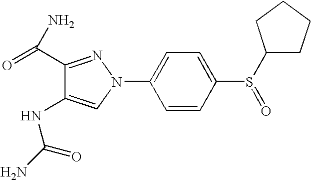 |
| 284 | 1-(3-carbamoyl-1-(4-(pyridin-2- ylsulfinyl)phenyl)-1H-pyrazol-4- yl)urea | 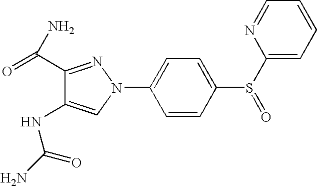 |
| 285 | 1-(1-(4-(2- hydroxyphenylsulfinyl)phenyl)-3- carbamoyl-1H-pyrazol-4-yl)urea | 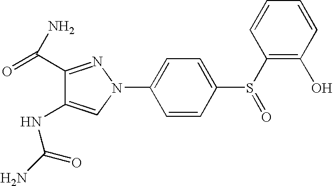 |
| 286 | 1-(3-carbamoyl-1-(4-(pyridin-2- ylsulfonyl)phenyl)-1H-pyrazol-4- yl)urea | 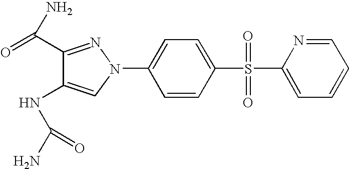 |
| 287 | 1-(3-carbamoyl-1-(4- (cyclopentylsulfonyl)phenyl)-1H- pyrazol-4-yl)urea | 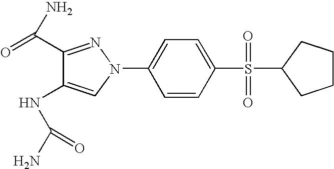 |
| 288 | 1-(1-(4-(2- hydroxyphenylsulfonyl)phenyl)-3- carbamoyl-1H-pyrazol-4-yl)urea | 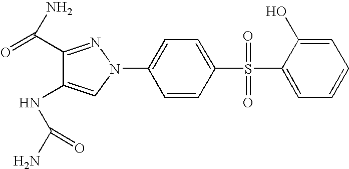 |
| 290 | 1-(1-(4-(4- chlorophenylthio)phenyl)-3- carbamoyl-1H-pyrazol-4-yl)urea | 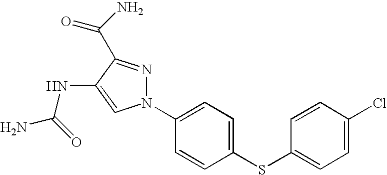 |
| 291 | 1-(3-carbamoyl-1-(4- (isopentylthio)phenyl)-1H-pyrazol- 4-yl)urea | 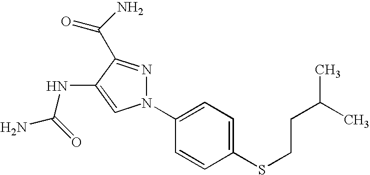 |
| 292 | 1-(1-(4-(m-tolylthio)phenyl)-3- carbamoyl-1H-pyrazol-4-yl)urea | 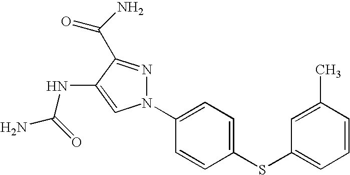 |
| 293 | 1-(1-(4-(3- bromophenylthio)phenyl)-3- carbamoyl-1H-pyrazol-4-yl)urea | 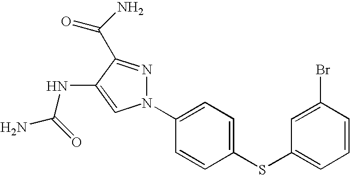 |
| 294 | 1-(3-carbamoyl-1-(4-(pyridin-4- ylsulfinyl)phenyl)-1H-pyrazol-4- yl)urea | 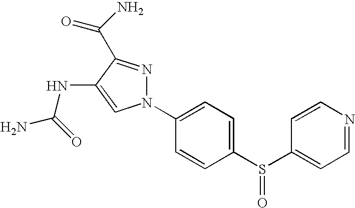 |
| 295 | 1-(3-carbamoyl-1-(4-(pyridin-4- ylsulfonyl)phenyl)-1H-pyrazol-4- yl)urea | 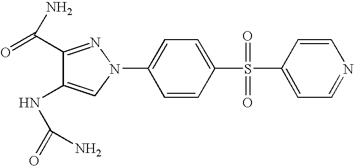 |
| 296 | 1-(2′-carbamoylmethoxy-4′-fluoro- 2-methyl-biphenyl-4-yl)-4-ureido- 1H-pyrazole-3-carboxylic acid amide | 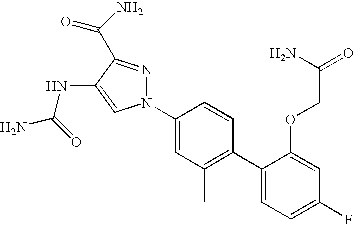 |
| 297 | 1-[4-(4-fluoro-phenylsulfanyl)-3- methyl-phenyl]-4-ureido-1H- pyrazole-3-carboxylic acid amide | 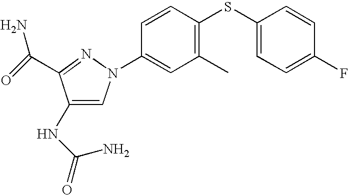 |
| 298 | 1-[4-(4-fluoro-benzenesulfonyl)-3- methyl-phenyl]-4-ureido-1H- pyrazole-3-carboxylic acid amide | 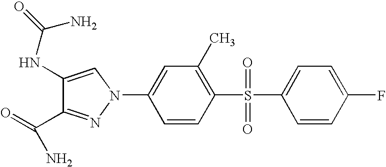 |
| 299 | 1-(3-hydroxy-phenyl)-4-ureido-1H- pyrazole-3-carboxylic acid 4- methoxy-benzylamide | 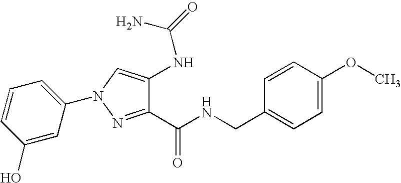 |
| 300 | 1-(3-hydroxy-phenyl)-4-ureido-1H- pyrazole-3-carboxylic acid amide | 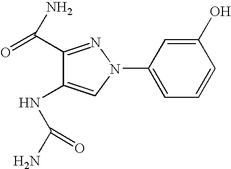 |
| 301 | 4-[(aminocarbonyl)amino]-1-[4- (aminosulfonyl)phenyl]-1H- pyrazole-3-carboxamide | 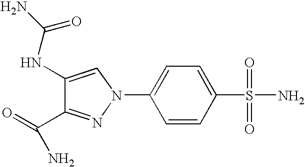 |
| 302 | 4-[(aminocarbonyl)aminol-1-(4- chloro-3-methylphenyl)-1H- pyrazole-3-carboxamide | 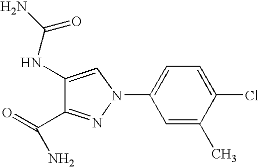 |
| 303 | 4-[(aminocarbonyl)amino]-1-[4- (piperidin-1-ylcarbonyl)phenyl]-1H- pyrazole-3-carboxamide | 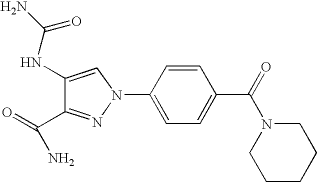 |
| 304 | 4-[(aminocarbonyl)aminol-1-(4- fluoro-3-methylphenyl)-1H- pyrazole-3-carboxamide | 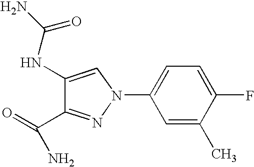 |
| 305 | 4-[(aminocarbonyl)amino]-1-(4- propylphenyl)-1H-pyrazole-3- carboxamide | 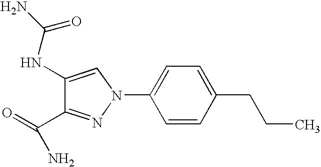 |
| 306 | 4-[(aminocarbonyl)amino]-1-(3- methyl-4-propoxyphenyl)-1H- pyrazole-3-carboxamide | 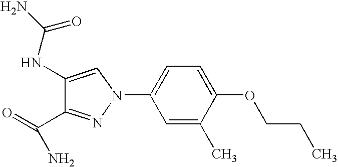 |
| 307 | 4-[(aminocarbonyl)amino]-1-[4- (benzyloxy)-3-methylphenyl]-1H- pyrazole-3-carboxamide | 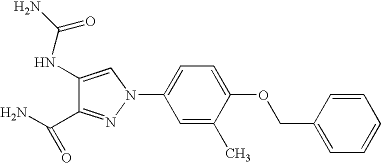 |
| 308 | 1-(4′-hydroxy-2-methyl-biphenyl-4- yl)-4-ureido-1H-pyrazole-3- carboxylic acid amide | 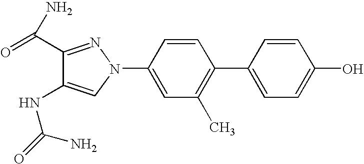 |
| 309 | 1-(4′-hydroxy-2,2′-dimethyl- biphenyl-4-yl)-4-ureido-1H- pyrazole-3-carboxylic acid amide | 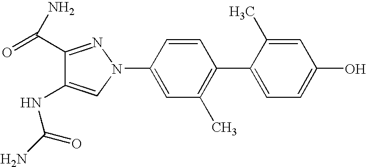 |
| 310 | 1-(3′-fluoro-4′-hydroxy-2-methyl- biphenyl-4-yl)-4-ureido-1H- pyrazole-3-carboxylic acid amide | 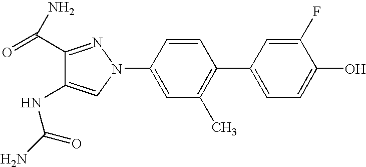 |
| 311 | 1-[4-(3-fluoro-phenylsulfanyl)-3- methyl-phenyl]-4-ureido-1H- pyrazole-3-carboxylic acid amide | 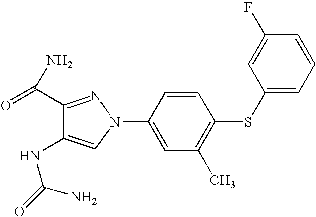 |
| 312 | 1-[4-(3-fluoro-benzenesulfonyl)-3- methyl-phenyl]-4-ureido-1H- pyrazole-3-carboxylic acid amide | 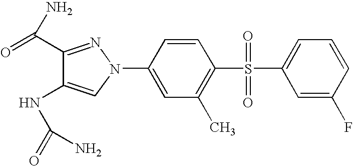 |
| 315 | 1-m-tolyl-4-ureido-1H-pyrazole-3- carboxylic acid amide | 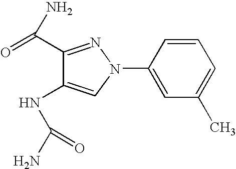 |
| 316 | 1-(3-fluoro-phenyl)-4-ureido-1H- pyrazole-3-carboxylic acid amide | 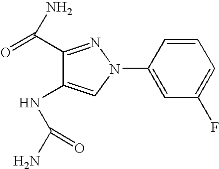 |
| 317 | 1-(3-trifluoromethyl-phenyl)-4- ureido-1H-pyrazole-3-carboxylic acid amide | 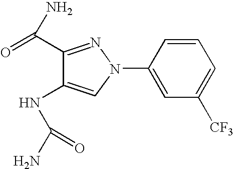 |
| 318 | 1-o-tolyl-4-ureido-1H-pyrazole-3- carboxylic acid amide | 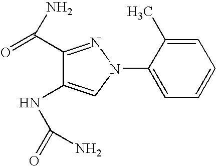 |
| 322 | 4-[(aminocarbonyl)amino]-1-(2- methylpyridin-4-yl)-1H-pyrazole-3- carboxamide | 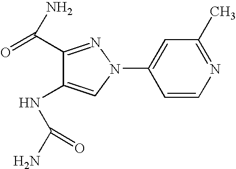 |
| 326 | 4-[(aminocarbonyl)amino]-1-(3- fluoro-4-methylphenyl)-1H- pyrazole-3-carboxamide | 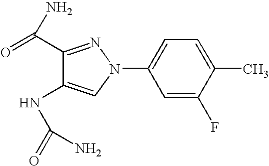 |
| 328 | 4-[(aminocarbonyl)amino]-1-[4- (trifluoromethoxy)phenyl]-1H- pyrazole-3-carboxamide | 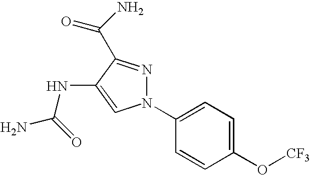 |
| 330 | 4-[(aminocarbonyl)aminol-1-(3,4- dimethoxyphenyl)-1H-pyrazole-3- carboxamide | 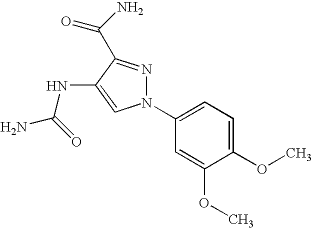 |
| 332 | 4-[(aminocarbonyl)amino]-1-(4- propoxyphenyl)-1H-pyrazole-3- carboxamide | 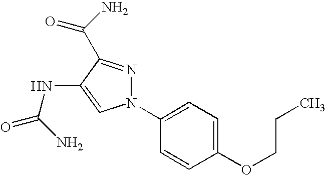 |
| 334 | 4-[(aminocarbonyl)amino]-1-(3- chloro-4-propoxyphenyl-1H- pyrazole-3-carboxamide | 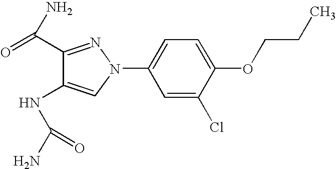 |
| 335 | 4-[(amiriocarbonyl)amino]-1-{3′-[2- (ethylamino)-2-oxoethoxy]-1,1′- biphenyl-4-yl}-1H-pyrazole-3- carboxamide | 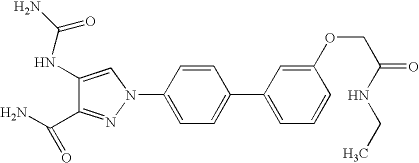 |
| 336 | 4-[(aminocarbonyl)amino]-1-{3′-[2- (diethylamino)-2-oxoethoxy]-1,1′- biphenyl-4-yl}-1H-pyrazole-3- carboxamide | 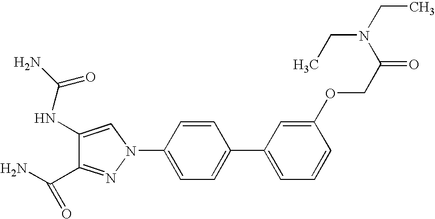 |
| 337 | 4-{3-(aminocarbonyl)-4- [(aminocarbonyl)amino]-1H- pyrazol-1-yl}phenylboronic acid | 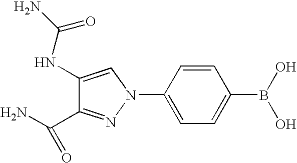 |
| 338 | 4-[(aminocarbonyl)amino]-1-[3′-(3- amino-3-oxopropyl)-1,1′-biphenyl- 4-yl]-1H-pyrazole-3-carboxamide | 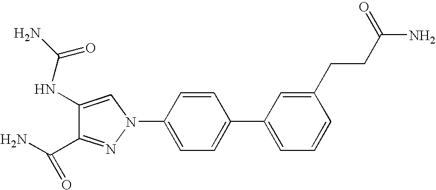 |
| 341 | 4-[(aminocarbonyl)amino]-1-(3′- cyano-1,1′-biphenyl-3-yl)-1H- pyrazole-3-carboxamide | 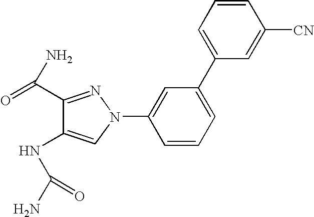 |
| 343 | 4-[(aminocarbonyl)amino]-1-(3′- methoxy-1,1′-biphenyl-3-yl)-1H- pyrazole-3-carboxamide | 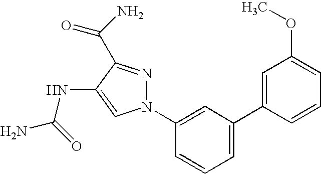 |
| 355 | 4-[(aminocarbonyl)amino]-1-(4′- ethoxy-1,1′-biphenyl-3-yl)-1H- pyrazole-3-carboxamide | 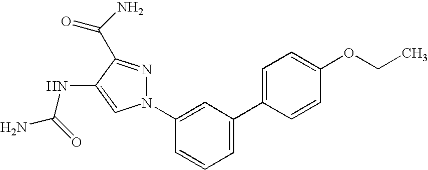 |
| 358 | 4-[(aminocarbonyl)aminol-1-[4′- (methylthio)-1,1′-biphenyl-3-yl]-1H- pyrazole-3-carboxamide | 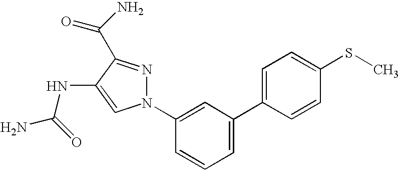 |
| 359 | 4-[(aminocarbonyl)amino]-1-(3′- fluoro-4′-methoxy-1,1′-biphenyl-3- yl)-1H-pyrazole-3-carboxamide | 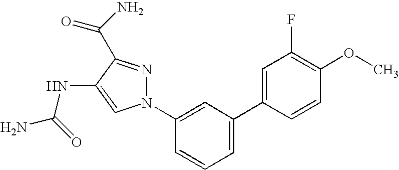 |
| 371 | 4-[(aminocarbonyl)amino]-1- (1,1′:2′,1″-terphenyl-3-yl)-1H- pyrazole-3-carboxamide | 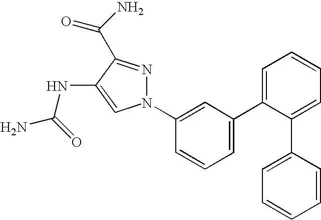 |
| 374 | 4-[(aminocarbonyl)amino]-1-[4′- (methylsulfonyl)-1,1′-biphenyl-3-yl]- 1H-pyrazole-3-carboxamide | 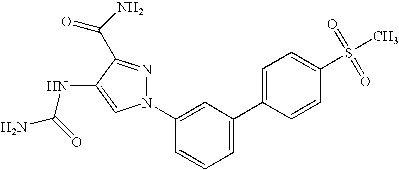 |
| 376 | 1-(1-(4-(4-fluorophenylthio)-2- chlorophenyl)-3-carbamoyl-1H- pyrazol-4-yl)urea | 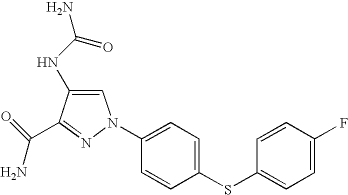 |
| 377 | 1-(1-(4-(4-fluorophenylsulfonyl)-2- chlorophenyl)-3-carbamoyl-1H- pyrazol-4-yl)urea | 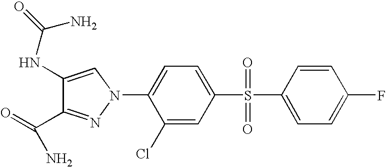 |
| 378 | 1-(1-(4-(4-fluorophenylsulfonyl)-2- fluorophenyl)-3-carbamoyl-1H- pyrazol-4-yl)urea | 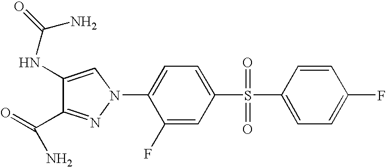 |
| 379 | 3-(4-{3-(aminocarbonyl)-4- [(aminocarbonyl)amino]-1H- pyrazol-1-yl}-1,1′-biphenyl-2- yl)propanoic acid | 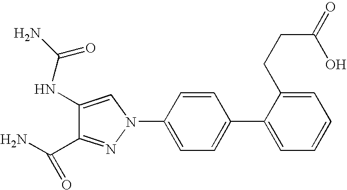 |
| 380 | 4-[(aminocarbonyl)amino]-1-(3′-{3- [(2,4-dimethoxybenzyl)amino]-3- oxopropyl}-1,1′-biphenyl-4-yl)-1H- pyrazole-3-carboxamide | 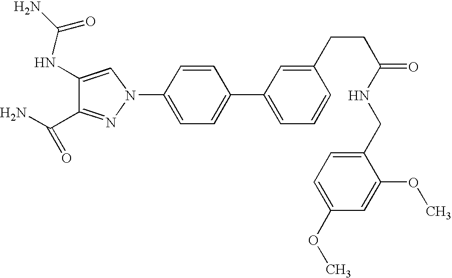 |
| 381 | 4-[(aminocarbonyl)amino]-1-{3′-[3- (methylamino)-3-oxopropyl]-1,1′- biphenyl-4-yl}-1H-pyrazole-3- carboxamide | 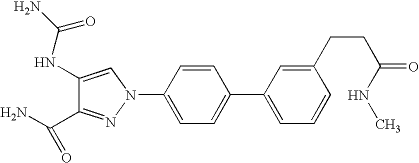 |
| 382 | 4-[(aminocarbonyl)amino]-1-{3′-[3- (diethylamino)-3-oxopropyl]-1,1′- biphenyl-4-yl}-1H-pyrazole-3- carboxamide | 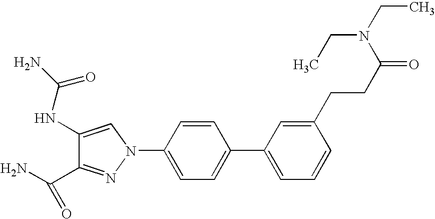 |
| 383 | 4-[(aminocarbonyl)amino]-1-{3′-[2- (methylamino)-2-oxoethoxy]-1,1′- biphenyl-4-yl}-1H-pyrazole-3- carboxamide | 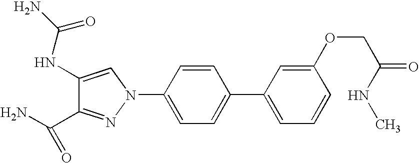 |

In one particularly preferred embodiment, the compound of Formula I is selected from the group of compounds consisting of:
4-[(aminocarbonyl)amino]-1-[4-bromo-3-(trifluoromethyl)phenyl]-1H-pyrazole-3-carboxamide;
4-[(aminocarbonyl)amino]-1-(4-bromo-3-ethoxyphenyl)-1H-pyrazole-3-carboxamide;
4-[(aminocarbonyl)amino]-1-(4-iodo-2-methylphenyl)-1H-pyrazole-3-carboxamide;
4-[(aminocarbonyl)amino]-1-(3′-hydroxy-3-methyl-1,1′-biphenyl-4-yl)-1H-pyrazole-3-carboxamide;
4-[(aminocarbonyl)amino]-1-(3-chloro-4-iodophenyl)-1H-pyrazole-3-carboxamide;
4-[(aminocarbonyl)amino]-1-(2-chloro-3′-hydroxy-1,1′-biphenyl-4-yl)-1H-pyrazole-3-carboxamide;
4-[(aminocarbonyl)amino]-1-(4-iodo-3-methylphenyl)-1H-pyrazole-3-carboxamide;
4-[(aminocarbonyl)amino]-1-[4-(morpholin-4-yl-carbonyl)phenyl]-1H-pyrazole-3-carboxamide;
4-[(aminocarbonyl)amino]-1-(4-methoxyphenyl)-1H-pyrazole-3-carboxamide trifluoroacetate;
4-[(aminocarbonyl)amino]-1-[3-(3,4-dimethoxybenzyl)phenyl]-1H-pyrazole-3-carboxamide;
4-[(aminocarbonothioyl)amino]-1-(3-bromophenyl)-1H-pyrazole-3-carboxamide;
4-[(aminocarbonyl)amino]-1-[3-(2-chloropyridin-4-yl)phenyl]-1H-pyrazole-3-carboxamide;
4-[(aminocarbonyl)amino]-1-(4-methoxyphenyl)-1H-pyrazole-3-carboxamide trifluoroacetate;
1-(3-bromophenyl)-4-{[(methylamino)carbonyl]amino}-1H-pyrazole-3-carboxamide;
1-(4-bromophenyl)-4-{[(methylamino)carbonyl]amino}-1H-pyrazole-3-carboxamide;
1-(4-iodophenyl)-4-{[(methylamino)carbonyl]amino}-1H-pyrazole-3-carboxamide;
4-[(aminocarbonyl)amino]-1-(4-methylphenyl)-1H-pyrazole-3-carboxamide;
4-[(aminocarbonyl)amino]-1-(4-bromophenyl)-1H-pyrazole-3-carboxamide;
4-[(aminocarbonyl)amino]-1-(3-bromophenyl)-1H-pyrazole-3-carboxamide;
4-[(aminocarbonyl)amino]-1-(4-pyridin-3-ylphenyl)-1H-pyrazole-3-carboxamide;
4-[(aminocarbonyl)amino]-1-(3′-hydroxy-1,1′-biphenyl-4-yl)-1H-pyrazole-3-carboxamide;
4-[(aminocarbonyl)amino]-1-(2′-hydroxy-1,1′-biphenyl-4-yl)-1H- pyrazole-3-carboxamide;
4-[(aminocarbonyl)amino]-1-[3′-(trifluoromethoxy)-1,1′-biphenyl-4-yl]-1H-pyrazole-3-carboxamide;
4-[(aminocarbonyl)amino]-1-(4′-fluoro-1,1′-biphenyl-4-yl)-1H-pyrazole-3-carboxamide;
4-[(aminocarbonyl)amino]-1-[4-(1H-pyrrol-2-yl)phenyl]-1H-pyrazole-3-carboxamide;
4-[(aminocarbonyl)amino]-1-[3′-(cyanomethyl)-1,1′-biphenyl-4-yl]-1H-pyrazole-3-carboxamide;
4-[(aminocarbonyl)amino]-1-[3′-(aminomethyl)-1,1′-biphenyl-4-yl]-1H-pyrazole-3-carboxamide;
methyl 2-amino-4′-{3-(aminocarbonyl)-4-[(aminocarbonyl)amino]-1H-pyrazol-1-yl}-1,1′-biphenyl-4-carboxylate;
4-[(aminocarbonyl)amino]-1-(2′-fluoro-1,1′-biphenyl-4-yl)-1H-pyrazole-3-carboxamide;
4-[(aminocarbonyl)amino]-1-{3′-[(tert-butylamino)carbonyl]-1,1′-biphenyl-4-yl}-1H-pyrazole-3-carboxamide;
4-[(aminocarbonyl)amino]-1-{3′-[(E)-2-cyanovinyl]-1,1′-biphenyl-4-yl}-1H-pyrazole-3-carboxamide;
4-[(aminocarbonyl)amino]-1-{4′-[(pyridin-2-ylamino)carbonyl]-1,1′-biphenyl-4-yl}-1H-pyrazole-3-carboxamide;
4-[(aminocarbonyl)amino]-1-[4′-(morpholin-4-ylmethyl)-1,1′-biphenyl-4-yl]-1H-pyrazole-3-carboxamide;
4-[(aminocarbonyl)amino]-1-{4′-[(benzylamino)carbonyl]-1,1′-biphenyl-4-yl}-1H-pyrazole-3-carboxamide;
4-[(aminocarbonyl)amino]-1-(4′-hydroxy-1,1′-biphenyl-4-yl)-1H-pyrazole-3-carboxamide;
4-[(aminocarbonyl)amino]-1-[4′-(cyanomethyl)-1,1′-biphenyl-4-yl]-1H-pyrazole-3-carboxamide;
4-[(aminocarbonyl)amino]-1-[4′-(hydroxymethyl)-1,1′-biphenyl-4-yl]-1H-pyrazole-3-carboxamide;
4-[(aminocarbonyl)amino]-1-[3′-(hydroxymethyl)-1,1′-biphenyl-4-yl]-1H-pyrazole-3-carboxamide;
4-[(aminocarbonyl)amino]-1-(4-pyridin-4-ylphenyl)-1H-pyrazole-3-carboxamide;
4-[(aminocarbonyl)amino]-1-(3′-fluoro-1,1′-biphenyl-3-yl)-1H-pyrazole-3-carboxamide;
4-[(aminocarbonyl)amino]-1-(4′-fluoro-1,1′-biphenyl-3-yl)-1H-pyrazole-3-carboxamide;
4-[(aminocarbonyl)amino]-1-(3-thien-3-ylphenyl)-1H-pyrazole-3-carboxamide;
4-[(aminocarbonyl)amino]-1-[4′-(cyanomethyl)-1,1′-biphenyl-3-yl]-1H-pyrazole-3-carboxamide;
4-[(aminocarbonyl)amino]-1-(3-pyridin-4-ylphenyl)-1H-pyrazole-3-carboxamide;
4-[(aminocarbonyl)amino]-1-(3-pyridin-4-ylphenyl)-1H-pyrazole-3-carboxamide;
4-[(aminocarbonyl)amino]-1-(4′-hydroxy-1,1′-biphenyl-3-yl)-1H-pyrazole-3-carboxamide;
4-[(aminocarbonyl)amino]-1-[4′-(hydroxymethyl)-1,1′-biphenyl-3-yl]-1H-pyrazole-3-carboxamide;
4-[(aminocarbonyl)amino]-1-[3′-(hydroxymethyl)-1,1′-biphenyl-3-yl]-1H-pyrazole-3-carboxamide;
1-(3′-amino-1,1′-biphenyl-3-yl)-4-[(aminocarbonyl)amino]-1H-pyrazole-3-carboxamide;
4-[(aminocarbonyl)amino]-1-[4′-(morpholin-4-ylmethyl)-1,1′-biphenyl-3-yl]-1H-pyrazole-3-carboxamide;
4-[(aminocarbonyl)amino]-1-[4′-(aminomethyl)-1,1′-biphenyl-3-yl]-1H-pyrazole-3-carboxamide;
1-(4′-acetyl-1,1′-biphenyl-3-yl)-4-[(aminocarbonyl)amino]-1H-pyrazole-3-carboxamide;
1-(4′-amino-1,1′-biphenyl-3-yl)-4-[(aminocarbonyl)amino]-1H-pyrazole-3-carboxamide;
1-[4′-(acetylamino)-1,1′-biphenyl-3-yl]-4-[(aminocarbonyl)amino]-1H-pyrazole-3-carboxamide;
4-[(aminocarbonyl)amino]-1-(3′,4′-dimethoxy-1,1′-biphenyl-3-yl)-1H-pyrazole-3-carboxamide;
4-[(aminocarbonyl)amino]-1-[3-(1H-pyrazol-4-yl)phenyl]-1H-pyrazole-3-carboxamide;
3′-{3-(aminocarbonyl)-4-[(aminocarbonyl)amino]-1H-pyrazol-1-yl}-1,1′-biphenyl-4-carboxylic acid;
4-[(aminocarbonyl)amino]-1-{4′-[(methylsulfonyl)amino]-1,1′-biphenyl-3-yl}-1H-pyrazole-3-carboxamide;
4-[(aminocarbonyl)amino]-1-(3-quinolin-5-ylphenyl)-1H-pyrazole-3-carboxamide;
4-[(aminocarbonyl)amino]-1-[4′-(dimethylamino)-1,1′-biphenyl-3-yl]-1H-pyrazole-3-carboxamide;
4-[(aminocarbonyl)amino]-1-[3-(1,3-benzodioxol-5-yl)phenyl]-1H-pyrazole-3-carboxamide;
4-[(aminocarbonyl)amino]-1-[3-(2-methylpyridin-4-yl)phenyl]-1H-pyrazole-3-carboxamide;
4-[(aminocarbonyl)amino]-1-[3-(4-methyl-3,4-dihydro-2H-1,4-benzoxazin-7-yl)phenyl]-1H-pyrazole-3-carboxamide;
4-[(aminocarbonyl)amino]-1-[3-(1H-indol-5-yl)phenyl]-1H-pyrazole-3-carboxamide;
4-[(aminocarbonyl)amino]-1-(3-pyrimidin-5-ylphenyl)-1H-pyrazole-3-carboxamide;
4-[(aminocarbonyl)amino]-1-{3-[(E)-2-phenylvinyl]phenyl}-1H-pyrazole-3-carboxamide;
4-[(aminocarbonyl)amino]-1-[3-(1H-pyrrol-2-yl)phenyl]-1H-pyrazole-3-carboxamide;
4-[(aminocarbonyl)amino]-1-[3-(3-methylpyridin-4-yl)phenyl]-1H-pyrazole-3-carboxamide;
4-[(aminocarbonyl)amino]-1-[3-(3,5-dimethylisoxazol-4-yl)phenyl]-1H-pyrazole-3-carboxamide;
4-[(aminocarbonyl)amino]-1-(2′-hydroxy-1,1′-biphenyl-3-yl)-1H-pyrazole-3-carboxamide;
4-[(aminocarbonyl)amino]-1-(3′-hydroxy-1,1′-biphenyl-3-yl)-1H-pyrazole-3-carboxamide;
3′-{3-(aminocarbonyl)-4-[(aminocarbonyl)amino]-1H-pyrazol-1-yl}-1,1′-biphenyl-3-carboxylic acid;
4-[(aminocarbonyl)amino]-1-{4′-[(methylamino)carbonyl]-1,1′-biphenyl-3-yl}-1H-pyrazole-3-carboxamide;
4-[(aminocarbonyl)amino]-1-{4′-[(propylamino)carbonyl]-1,1′-biphenyl-3-yl}-1H-pyrazole-3-carboxamide;
1-[3′-(acetylamino)-1,1′-biphenyl-3-yl]-4-[(aminocarbonyl)amino]-1H-pyrazole-3-carboxamide;
4-[(aminocarbonyl)amino]-1-(3′-amino-4′-methyl-1,1′-biphenyl-3-yl)-1H-pyrazole-3-carboxamide;
4-[(aminocarbonyl)amino]-1-[3′-(aminomethyl)-1,1′-biphenyl-3-yl]-1H-pyrazole-3-carboxamide;
4-[(aminocarbonyl)amino]-1-[3′-(aminocarbonyl)-1,1′-biphenyl-3-yl]-1H-pyrazole-3-carboxamide;
4-[(aminocarbonyl)amino]-1-{4′-[(cyclopropylamino)carbonyl]-1,1′-biphenyl-3-yl}-1H-pyrazole-3-carboxamide;
4-[(aminocarbonyl)amino]-1-{3′-[(cyclopropylamino)carbonyl]-1,1′-biphenyl-3-yl}-1H-pyrazole-3-carboxamide;
4-[(aminocarbonyl)amino]-1-(3′-{[(2-cyanoethyl)amino]carbonyl}-1,1′-biphenyl-3-yl)-1H-pyrazole-3-carboxamide;
4-[(aminocarbonyl)amino]-1-{3′-[(methylamino)carbonyl]-1,1′-biphenyl-3-yl}-1H-pyrazole-3-carboxamide;
4-[(aminocarbonyl)amino]-1-(4′-{[(2-cyanoethyl)amino]carbonyl}-1,1′-biphenyl-3-yl)-1H-pyrazole-3-carboxamide;
N-[(3′-{3-(aminocarbonyl)-4-[(aminocarbonyl)amino]-1H-pyrazol-1-yl}-1,1′-biphenyl-4-yl)carbonyl]glycine;
4-[(aminocarbonyl)amino]-1-[2′-(benzyloxy)-1,1′-biphenyl-3-yl]-1H-pyrazole-3-carboxamide;
4-[(aminocarbonyl)amino]-1-[2′-(hydroxymethyl)-1,1′-biphenyl-3-yl]-1H-pyrazole-3-carboxamide;
4-[(aminocarbonyl)amino]-1-(2′-methoxy-1,1′-biphenyl-3-yl)-1H-pyrazole-3-carboxamide;
4-[(aminocarbonyl)amino]-1-(4′-hydroxy-2′-methyl-1,1′-biphenyl-3-yl)-1H-pyrazole-3-carboxamide;
4-[(aminocarbonyl)amino]-1-{4′-[(4-oxopiperidin-1-yl)carbonyl]-1,1′-biphenyl-3-yl}-1H-pyrazole-3-carboxamide;
1-(2′-acetyl-1,1′-biphenyl-3-yl)-4-[(aminocarbonyl)amino]-1H-pyrazole-3-carboxamide;
4-[(aminocarbonyl)amino]-1-(4′-fluoro-2′-hydroxy-1,1′-biphenyl-3-yl)-1H-pyrazole-3-carboxamide;
4-[(aminocarbonyl)amino]-1-(5′-fluoro-2′-hydroxy-1,1′-biphenyl-3-yl)-1H-pyrazole-3-carboxamide;
4-[(aminocarbonyl)amino]-1-[2′-(cyanomethoxy)-5′-fluoro-1,1′-biphenyl-3-yl]-1H-pyrazole-3-carboxamide;
1-[4′-(aminocarbonyl)-1,1′-biphenyl-4-yl]-4-{[(methylamino)carbonyl]amino}-1H-pyrazole-3-carboxamide;
4′-{3-(aminocarbonyl)-4-[(aminocarbonyl)amino]-1H-pyrazol-1-yl}-1,1′-biphenyl-3-carboxylic acid;
4′-{3-(aminocarbonyl)-4-[(aminocarbonyl)amino]-1H-pyrazol-1-yl}-3-fluoro-1,1′-biphenyl-4-carboxylic acid;
4-[(aminocarbonyl)amino]-1-(4′-fluoro-2′-hydroxy-1,1′-biphenyl-4-yl)-1H-pyrazole-3-carboxamide;
4-[(aminocarbonyl)amino]-1-(5′-fluoro-2′-hydroxy-1,1′-biphenyl-4-yl)-1H-pyrazole-3-carboxamide;
4-[(aminocarbonyl)amino]-1-(4′-cyano-1,1′-biphenyl-4-yl)-1H-pyrazole-3-carboxamide;
4-[(aminocarbonyl)amino]-1-(1,1′-biphenyl-4-yl)-1H-pyrazole-3-carboxamide;
4-[(aminocarbonyl)amino]-1-(3′-methyl-1,1′-biphenyl-4-yl)-1H-pyrazole-3-carboxamide;
4-[(aminocarbonyl)amino]-1-(3′-cyano-1,1′-biphenyl-4-yl)-1H-pyrazole-3-carboxamide;
4-[(aminocarbonyl)amino]-1-[2′-(hydroxymethyl)-1,1′-biphenyl-4-yl]-1H-pyrazole-3-carboxamide;
4-[(aminocarbonyl)amino]-1-(2′-methoxy-1,1′-biphenyl-4-yl)-1H-pyrazole-3-carboxamide;
4-[(aminocarbonyl)amino]-1-(3′-methoxy-1,1′-biphenyl-4-yl)-1H-pyrazole-3-carboxamide;
4-[(aminocarbonyl)amino]-1-(4′-fluoro-2′-methyl-1,1′-biphenyl-4-yl)-1H-pyrazole-3-carboxamide;
4-[(aminocarbonyl)amino]-1-(4′-fluoro-3′-methyl-1,1′-biphenyl-4-yl)-1H-pyrazole-3-carboxamide;
4-[(aminocarbonyl)amino]-1-(2′-chloro-1,1′-biphenyl-4-yl)-1H-pyrazole-3-carboxamide;
4-[(aminocarbonyl)amino]-1-(3′-chloro-1,1′-biphenyl-4-yl)-1H-pyrazole-3-carboxamide;
4-[(aminocarbonyl)amino]-1-(2′,3′-difluoro-1,1′-biphenyl-4-yl)-1H-pyrazole-3-carboxamide;
4-[(aminocarbonyl)amino]-1-(2′,4′-difluoro-1,1′-biphenyl-4-yl)-1H-pyrazole-3-carboxamide;
4-[(aminocarbonyl)amino]-1-(3′,4′-difluoro-1,1′-biphenyl-4-yl)-1H-pyrazole-3-carboxamide;
4-[(aminocarbonyl)amino]-1-(3′,5′-difluoro-1,1′-biphenyl-4-yl)-1H-pyrazole-3-carboxamide;
4-[(aminocarbonyl)amino]-1-[3′-(methylthio)-1,1′-biphenyl-4-yl]-1H-pyrazole-3-carboxamide;
4-[(aminocarbonyl)amino]-1-[4′-(methylthio)-1,1′-biphenyl-4-yl]-1H-pyrazole-3-carboxamide;
4-[(aminocarbonyl)amino]-1-(2′-fluoro-3′-methoxy-1,1′-biphenyl-4-yl)-1H-pyrazole-3-carboxamide;
4-[(aminocarbonyl)amino]-1-(5′-fluoro-2′-methoxy-1,1′-biphenyl-4-yl)-1H-pyrazole-3-carboxamide;
4-[(aminocarbonyl)amino]-1-(3′-fluoro-4′-methoxy-1,1′-biphenyl-4-yl)-1H-pyrazole-3-carboxamide;
4-[(aminocarbonyl)amino]-1-[4-(1-naphthyl)phenyl]-1H-pyrazole-3-carboxamide;
4-[(aminocarbonyl)amino]-1-(3′-chloro-4′-fluoro-1,1′-biphenyl-4-yl)-1H-pyrazole-3-carboxamide;
4-[(aminocarbonyl)amino]-1-(5′-chloro-2′-methoxy-1,1′-biphenyl-4-yl)-1H-pyrazole-3-carboxamide;
4-[(aminocarbonyl)amino]-1-[3′-(1H-pyrazol-1-yl)-1,1′-biphenyl-4-yl]-1H-pyrazole-3-carboxamide;
4-[(aminocarbonyl)amino]-1-[3′-(trifluoromethyl)-1,1′-biphenyl-4-yl]-1H-pyrazole-3-carboxamide;
4-[(aminocarbonyl)amino]-1-(2′,3′-dichloro-1,1′-biphenyl-4-yl)-1H-pyrazole-3-carboxamide;
4-[(aminocarbonyl)amino]-1-(2′,4′-dichloro-1,1′-biphenyl-4-yl)-1H-pyrazole-3-carboxamide;
4-[(aminocarbonyl)amino]-1-(2′,5′-dichloro-1,1′-biphenyl-4-yl)-1H-pyrazole-3-carboxamide;
4-[(aminocarbonyl)amino]-1-(1,1′:3′,1″-terphenyl-4-yl)-1H-pyrazole-3-carboxamide;
4′-{3-(aminocarbonyl)-4-[(aminocarbonyl)amino]-1H-pyrazol-1-yl}-1,1′-biphenyl-4-carboxylic acid;
4-[(aminocarbonyl)amino]-1-{4′-[(diethylamino)carbonyl]-1,1′-biphenyl-4-yl}-1H-pyrazole-3-carboxamide;
ethyl 4′-{3-(aminocarbonyl)-4-[(aminocarbonyl)amino]-1H-pyrazol-1-yl}-1,1′-biphenyl-3-carboxylate;
4-[(aminocarbonyl)amino]-1-{4′-[(methylamino)carbonyl]-1,1′-biphenyl-4-yl}-1H-pyrazole-3-carboxamide;
4-[(aminocarbonyl)amino]-1-{4′-[(propylamino)carbonyl]-1,1′-biphenyl-4-yl}-1H-pyrazole-3-carboxamide;
ethyl 4′-{3-(aminocarbonyl)-4-[(aminocarbonyl)amino]-1H-pyrazol-1-yl}-1,1′-biphenyl-4-carboxylate;
4-[(aminocarbonyl)amino]-1-{3′-[(isopropylamino)carbonyl]-1,1′-biphenyl-4-yl}-1H-pyrazole-3-carboxamide;
4-[(aminocarbonyl)amino]-1-[4′-(aminocarbonyl)-1,1′-biphenyl-4-yl]-1H-pyrazole-3-carboxamide;
4-[(aminocarbonyl)amino]-1-(3′-fluoro-4′-{[(4-methoxybenzyl)amino]carbonyl}-1,1′-biphenyl-4-yl)-1H-pyrazole-3-carboxamide;
4-[(aminocarbonyl)amino]-1-{4′-[(benzylamino)carbonyl]-3′-fluoro-1,1′-biphenyl-4-yl}-1H-pyrazole-3-carboxamide;
4-[(aminocarbonyl)amino]-1-{3′-fluoro-4′-[(methylamino)carbonyl]-1,1′-biphenyl-4-yl}-1H-pyrazole-3-carboxamide;
4-[(aminocarbonyl)amino]-1-(3′-fluoro-4′-{[(3-methoxybenzyl)amino]carbonyl}-1,1′-biphenyl-4-yl)-1H-pyrazole-3-carboxamide;
4-[(aminocarbonyl)amino]-1-(3′-fluoro-4′-{[(2-methoxybenzyl)amino]carbonyl}-1,1′-biphenyl-4-yl)-1H-pyrazole-3-carboxamide;
4-[(aminocarbonyl)amino]-1-(3′-fluoro-4′-{[(4-fluorobenzyl)amino]carbonyl}-1,1′-biphenyl-4-yl)-1H-pyrazole-3-carboxamide;
4-[(aminocarbonyl)amino]-1-(3′-fluoro-4′-{[(3-fluorobenzyl)amino]carbonyl}-1,1′-biphenyl-4-yl)-1H-pyrazole-3-carboxamide;
4-[(aminocarbonyl)amino]-1-(3′-fluoro-4′-{[(2-fluorobenzyl)amino]carbonyl}-1,1′-biphenyl-4-yl)-1H-pyrazole-3-carboxamide;
4-[(aminocarbonyl)amino]-1-(3′-fluoro-4′-{[(4-fluorobenzyl)(methyl)amino]carbonyl}-1,1′-biphenyl-4-yl)-1H-pyrazole-3-carboxamide;
4-[(aminocarbonyl)amino]-1-{3′-fluoro-4′-[(1,2,3,4-tetrahydronaphthalen-1-ylamino)carbonyl]-1,1′-biphenyl-4-yl}-1H-pyrazole-3-carboxamide;
4-[(aminocarbonyl)amino]-1-(4′-{[[2-(dimethylamino)ethyl](methyl)amino]carbonyl}-3′-fluoro-1,1′-biphenyl-4-yl)-1H-pyrazole-3-carboxamide;
4-[(aminocarbonyl)amino]-1-[5′-fluoro-2′-(2-morpholin-4-yl-2-oxoethoxy)-1,1′-biphenyl-4-yl]-1H-pyrazole-3-carboxamide;
4′-{3-(aminocarbonyl)-4-[(aminocarbonyl)amino]-1H-pyrazol-1-yl}-1,1′-biphenyl-3-yl)methyl acetate;
4-[(aminocarbonyl)amino]-1-[3′-(cyanomethoxy)-1,1′-biphenyl-4-yl]-1H-pyrazole-3-carboxamide;
4-[(aminocarbonyl)amino]-1-[2′-(cyanomethoxy)-1,1′-biphenyl-4-yl]-1H-pyrazole-3-carboxamide;
4-[(aminocarbonyl)amino]-1-[3′-(pyridin-2-ylmethoxy)-1,1′-biphenyl-4-yl]-1H-pyrazole-3-carboxamide;
4-[(aminocarbonyl)amino]-1-[3′-(1-cyanoethoxy)-1,1′-biphenyl-4-yl]-1H-pyrazole-3-carboxamide;
4-[(aminocarbonyl)amino]-1-[3′-(pyridin-3-ylmethoxy)-1,1′-biphenyl-4-yl]-1H-pyrazole-3-carboxamide;
4-[(aminocarbonyl)amino]-1-[3′-(pyridin-4-ylmethoxy)-1,1′-biphenyl-4-yl]-1H-pyrazole-3-carboxamide;
4-[(aminocarbonyl)amino]-1-[2′-(cyanomethoxy)-5′-fluoro-1,1′-biphenyl-4-yl]-1H-pyrazole-3-carboxamide;
4-[(aminocarbonyl)amino]-1-[2′-(cyanomethoxy)-4′-fluoro-111′-biphenyl-4-yl]-1H-pyrazole-3-carboxamide;
tert-butyl [(4′-{3-(aminocarbonyl)-4-[(aminocarbonyl)amino]-1H-pyrazol-1-yl}-1,1′-biphenyl-3-yl)oxy]acetate;
4-[(aminocarbonyl)amino]-1-[3′-(2-amino-2-oxoethoxy)-1,1′-biphenyl-4-yl]-1H-pyrazole-3-carboxamide;
4-[(aminocarbonyl)amino]-1-[2′-(cyanomethoxy)-1,1′-biphenyl-3-yl]-1H-pyrazole-3-carboxamide;
4-[(aminocarbonyl)amino]-1-[4′-(cyanomethoxy)-1,1′-biphenyl-3-yl]-1H-pyrazole-3-carboxamide;
4-[(aminocarbonyl)amino]-1-[3′-(cyanomethoxy)-1,1′-biphenyl-3-yl]-1H-pyrazole-3-carboxamide;
4-[(aminocarbonyl)amino]-1-[3′-(cyanomethoxy)-3-fluoro-1,1′-biphenyl-4-yl]-1H-pyrazole-3-carboxamide;
4-[(aminocarbonyl)amino]-1-[2′-(1-cyanoethoxy)-5′-fluoro-1,1′-biphenyl-4-yl]-1H-pyrazole-3-carboxamide;
4-[(aminocarbonyl)amino]-1-{3′-[2-(dimethylamino)-2-oxoethoxy]-1,1′-biphenyl-4-yl}-1H-pyrazole-3-carboxamide;
4-[(aminocarbonyl)amino]-1-[4′-(aminocarbonyl)-3′-fluoro-1,1′-biphenyl-4-yl]-1H-pyrazole-3-carboxamide;
methyl 4-{3-(aminocarbonyl)-4-[(aminocarbonyl)amino]-1H-pyrazol-1-yl}benzoate;
methyl 3-{3-(aminocarbonyl)-4-[(aminocarbonyl)amino]-1H-pyrazol-1-yl}benzoate;
4-[(aminocarbonyl)amino]-1-[3′-(2-cyanoethyl)-1,1′-biphenyl-4-yl]-1H-pyrazole-3-carboxamide;
4-[(aminocarbonyl)amino]-1-[4′-(1H-tetrazol-5-yl)-1,1′-biphenyl-4-yl]-1H-pyrazole-3-carboxamide;
4-[(aminocarbonyl)amino]-1-(3,4-dichlorophenyl)-1H-pyrazole-3-carboxamide;
1-(3-carbamoyl-1-(4-(cyclohexylthio)phenyl)-1H-pyrazol-4-yl)urea;
1-(1-(4-(2-fluorophenylthio)phenyl)-3-carbamoyl-1H-pyrazol-4-yl)urea;
1-(3-carbamoyl-1-(4-(cyclohexylsulfinyl)phenyl)-1H-pyrazol-4-yl)urea;
1-(3-carbamoyl-1-(4-(cyclohexylsulfonyl)phenyl)-1H-pyrazol-4-yl)urea;
1-(1-(4-(2-fluorophenylsulfinyl)phenyl)-3-carbamoyl-1H-pyrazol-4-yl)urea;
1-(1-(4-(2-fluorophenylsulfonyl)phenyl)-3-carbamoyl-1H-pyrazol-4-yl)urea;
1-(3-carbamoyl-1-(3-iodophenyl)-1H-pyrazol-4-yl)urea;
1-(1-(4-(4-fluorophenylthio)phenyl)-3-carbamoyl-1H-pyrazol-4-yl)urea;
1-(1-(4-(3-fluorophenylthio)phenyl)-3-carbamoyl-1H-pyrazol-4-yl)urea;
1-(1-(3-(2-fluorophenylthio)phenyl)-3-carbamoyl-1H-pyrazol-4-yl)urea;
1-(1-(4-(4-hydroxyphenylthio)phenyl)-3-carbamoyl-1H-pyrazol-4-yl)urea;
1-(1-(4-(4-fluorophenylsulfinyl)phenyl)-3-carbamoyl-1H-pyrazol-4-yl)urea;
1-(1-(4-(3-fluorophenylsulfinyl)phenyl)-3-carbamoyl-1H-pyrazol-4-yl)urea;
1-(1-(3-(2-fluorophenylsulfinyl)phenyl)-3-carbamoyl-1H-pyrazol-4-yl)urea;
1-(1-(4-(3-fluorophenylsulfonyl)phenyl)-3-carbamoyl-1H-pyrazol-4-yl)urea;
1-(1-(4-(4-fluorophenylsulfonyl)phenyl)-3-carbamoyl-1H-pyrazol-4-yl)urea;
1-(3-carbamoyl-1-(4-(pyridin-2-ylthio)phenyl)-1H-pyrazol-4-yl)urea;
1-(3-carbamoyl-1-(4-(pyridin-4-ylthio)phenyl)-1H-pyrazol-4-yl)urea;
1-(3-carbamoyl-1-(4-(cyclopentylthio)phenyl)-1H-pyrazol-4-yl)urea;
1-(1-(4-(2-hydroxyphenylthio)phenyl)-3-carbamoyl-1H-pyrazol-4-yl)urea;
4-[(Aminocarbonyl)amino]-1-(3-chloro-4-methoxyphenyl)-1H-pyrazole-3-carboxamide;
1-(1-(4-(2-fluorophenylthio)-2-fluorophenyl)-3-carbamoyl-1H-pyrazol-4-yl)urea;
4-[(aminocarbonyl)amino]-1-(4-fluorophenyl)-1H-pyrazole-3-carboxamide;
4-[(aminocarbonyl)amino]-1-(1,3-benzodioxol-5-yl)-1H-pyrazole-3-carboxamide;
4-[(aminocarbonyl)amino]-1-(4-chlorophenyl)-1H-pyrazole-3-carboxamide;
4-[(aminocarbonyl)amino]-1-(4-iodophenyl)-1H-pyrazole-3-carboxamide;
4-[(aminocarbonyl)amino]-1-(4-isopropylphenyl)-1H-pyrazole-3-carboxamide;
4-[(aminocarbonyl)amino]-1-(1H-indol-5-yl)-1H-pyrazole-3-carboxamide;
4-[(aminocarbonyl)amino]-1-(3,4-difluorophenyl)-1H-pyrazole-3-carboxamide;
4-[(aminocarbonyl)amino]-1-(2-naphthyl)-1H-pyrazole-3-carboxamide;
4-[(aminocarbonyl)amino]-1-[2-chloro-3′-(cyanomethoxy)-1,1′-biphenyl-4-yl]-1H-pyrazole-3-carboxamide;
4-[(aminocarbonyl)amino]-1-{3-chloro-4-[(4-fluorophenyl)thio]phenyl}-1H-pyrazole-3-carboxamide;
4-[(aminocarbonyl)amino]-1-(2-chloro-2′-hydroxy-1,1′-biphenyl-4-yl)-1H-pyrazole-3-carboxamide;
4-[(aminocarbonyl)amino]-1-[2-chloro-2′-(cyanomethoxy)-1,1′-biphenyl-4-yl]-1H-pyrazole-3-carboxamide;
4-[(aminocarbonyl)amino]-1-(4-bromo-3-fluorophenyl)-1H-pyrazole-3-carboxamide;
4-[(aminocarbonyl)amino]-1-(2-fluoro-3′-hydroxy-1,1′-biphenyl-4-yl)-1H-pyrazole-3-carboxamide;
4-[(aminocarbonyl)amino]-1-[3′-(cyanomethoxy)-2-fluoro-1,1′-biphenyl-4-yl]-1H-pyrazole-3-carboxamide;
4-[(aminocarbonyl)amino]-1-{4-[(4-fluorophenyl)sulfonyl]-2-methylphenyl}-1H-pyrazole-3-carboxamide;
4-[(aminocarbonyl)amino]-1-(2-ethoxy-3′-hydroxy-1,1′-biphenyl-4-yl)-1H-pyrazole-3-carboxamide;
4-[(aminocarbonyl)amino]-1-[3′-(cyanomethoxy)-2-ethoxy-1,1′-biphenyl-4-yl]-1H-pyrazole-3-carboxamide;
4-[(aminocarbonyl)amino]-1-[3′-hydroxy-2-(trifluoromethyl)-1,1′-biphenyl-4-yl]-1H-pyrazole-3-carboxamide;
4-[(aminocarbonyl)amino]-1-[2′-hydroxy-2-(trifluoromethyl)-1,1′-biphenyl-4-yl]-1H-pyrazole-3-carboxamide;
4-[(aminocarbonyl)amino]-1-[2′-(cyanomethoxy)-2-ethoxy-1,1′-biphenyl-4-yl]-1H-pyrazole-3-carboxamide;
4-[(aminocarbonyl)amino]-1-[3′-(cyanomethoxy)-2-(trifluoromethyl)-1,1′-biphenyl-4-yl]-1H-pyrazole-3-carboxamide;
4-[(aminocarbonyl)amino]-1-[2′-(cyanomethoxy)-2-(trifluoromethyl)-1,1′-biphenyl-4-yl]-1H-pyrazole-3-carboxamide;
1-(3′-hydroxy-2-methyl-biphenyl-4-yl)-4-ureido-1H-pyrazole-3-carboxylic acid amide;
1-(2′-Hydroxy-2-methyl-biphenyl-4-yl)-4-ureido-1H-pyrazole-3-carboxylic acid amide;
1-(4′-Fluoro-2′-hydroxy-2-methyl-biphenyl-4-yl)-4-ureido-1H-pyrazole-3-carboxylic acid amide;
1-(5′-Fluoro-2′-hydroxy-2-methyl-biphenyl-4-yl)-4-ureido-1H-pyrazole-3-carboxylic acid amide;
1-(3′-Cyanomethoxy-2-methyl-biphenyl-4-yl)-4-ureido-1H-pyrazole-3-carboxylic acid amide;
1-(2′-Cyanomethoxy-4′-fluoro-2-methyl-biphenyl-4-yl)-4-ureido-1H-pyrazole-3-carboxylic acid amide;
1-(4-Iodo-3-methyl-phenyl)-4-(3-methyl-ureido)-1H-pyrazole-3-carboxylic acid amide;
1-(5-chloro-2-fluoro-phenyl)-4-ureido-1H-pyrazole-3-carboxylic acid amide;
4-[(aminocarbonyl)amino]-1-(3-fluoro-3′-hydroxy-1,1′-biphenyl-4-yl)-1H-pyrazole-3-carboxamide;
4′-{3-(aminocarbonyl)-4-[(aminocarbonyl)amino]-1H-pyrazol-1-yl}-3,3′-difluoro-1,1′-biphenyl-4-carboxylic acid;
4-[(aminocarbonyl)amino]-1-(3-fluoro-2′-hydroxy-1,1′-biphenyl-4-yl)-1H-pyrazole-3-carboxamide;
4-[(aminocarbonyl)amino]-1-[2′-(cyanomethoxy)-3-fluoro-1,1′-biphenyl-4-yl]-1H-pyrazole-3-carboxamide;
4-[(aminocarbonyl)amino]-1-(2-chloro-4-iodophenyl)-1H-pyrazole-3-carboxamide;
4-[(aminocarbonyl)amino]-1-[2′-(2-amino-2-oxoethoxy)-5′-fluoro-1,1′-biphenyl-4-yl]-1H-pyrazole-3-carboxamide;
4-[(aminocarbonyl)amino]-1-[3′-(2-oxopropoxy)-1,1′-biphenyl-4-yl]-1H-pyrazole-3-carboxamide;
4-[(aminocarbonyl)amino]-1-[3′-(1,3-oxazol-2-ylmethoxy)-1,1′-biphenyl-4-yl]-1H-pyrazole-3-carboxamide;
4-[(aminocarbonyl)amino]-1-[5′-fluoro-2′-(1,3-oxazol-2-ylmethoxy)-1,1′-biphenyl-4-yl]-1H-pyrazole-3-carboxamide;
4-[(aminocarbonyl)amino]-1-{5′-fluoro-2′-[2-(methylamino)-2-oxoethoxy]-1,1′-biphenyl-4-yl}-1H-pyrazole-3-carboxamide;
4-[(aminocarbonyl)amino]-1-{5′-fluoro-2′-[2-(ethylamino)-2-oxoethoxy]-1,1′-biphenyl-4-yl}-1H-pyrazole-3-carboxamide;
[(4′-{3-(aminocarbonyl)-4-[(aminocarbonyl)amino]-1H-pyrazol-1-yl}-1,1′-biphenyl-3-yl)oxy]acetic acid;
[(4′-{3-(aminocarbonyl)-4-[(aminocarbonyl)amino]-1H-pyrazol-1-yl}-5-fluoro-1,1′-biphenyl-2-yl)oxy]acetic acid;
4-[(aminocarbonyl)amino]-1-[4-(4,4,5,5-tetramethyl-1,3,2-dioxaborolan-2-yl)phenyl]-1H-pyrazole-3-carboxamide;
4-[(aminocarbonyl)amino]-1-(2-fluoro-2′-hydroxy-1,1′-biphenyl-4-yl)-1H-pyrazole-3-carboxamide;
4-[(aminocarbonyl)amino]-1-(2,4′-difluoro-2′-hydroxy-1,1′-biphenyl-4-yl)-1H-pyrazole-3-carboxamide;
4-[(aminocarbonyl)amino]-1-{3-chloro-4-[(2-fluorophenyl)thio]phenyl}-1H-pyrazole-3-carboxamide;
(2E)-2-[(4-bromo-2-ethylphenyl)hydrazono]-2-cyanoacetamide;
1-(3-carbamoyl-1-(4-(cyclopentylsulfinyl)phenyl)-1H-pyrazol-4-yl)urea;
1-(3-carbamoyl-1-(4-(pyridin-2-ylsulfinyl)phenyl)-1H-pyrazol-4-yl)urea;
1-(1-(4-(2-hydroxyphenylsulfinyl)phenyl)-3-carbamoyl-1H-pyrazol-4-yl)urea;
1-(3-carbamoyl-1-(4-(pyridin-2-ylsulfonyl)phenyl)-1H-pyrazol-4-yl)urea;
1-(3-carbamoyl-1-(4-(cyclopentylsulfonyl)phenyl)-1H-pyrazol-4-yl)urea;
1-(1-(4-(2-hydroxyphenylsulfonyl)phenyl)-3-carbamoyl-1H-pyrazol-4-yl)urea;
1-(1-(4-(4-chlorophenylthio)phenyl)-3-carbamoyl-1H-pyrazol-4-yl)urea;
1-(3-carbamoyl-1-(4-(isopentylthio)phenyl)-1H-pyrazol-4-yl)urea;
1-(2′-carbamoylmethoxy-4′-fluoro-2-methyl-biphenyl-4-yl)-4-ureido-1H-pyrazole-3-carboxylic acid amide;
1-[4-(4-fluoro-phenylsulfanyl)-3-methyl-phenyl]-4-ureido-1H-pyrazole-3-carboxylic acid amide;
1-[4-(4-fluoro-benzenesulfonyl)-3-methyl-phenyl]-4-ureido-1H-pyrazole-3-carboxylic acid amide;
1-(3-hydroxy-phenyl)-4-ureido-1H-pyrazole-3-carboxylic acid 4-methoxy-benzylamide;
4-[(aminocarbonyl)amino]-1-[4-(aminosulfonyl)phenyl]-1H-pyrazole-3-carboxamide;
4-[(aminocarbonyl)amino]-1-(4-chloro-3-methylphenyl)-1H-pyrazole-3-carboxamide;
4-[(aminocarbonyl)amino]-1-[4-(piperidin-1-ylcarbonyl)phenyl]-1H-pyrazole-3-carboxamide;
4-[(aminocarbonyl)amino]-1-(2-methylpyridin-4-yl)-1H-pyrazole-3-carboxamide;
4-[(aminocarbonyl)amino]-1-{3′-[2-(ethylamino)-2-oxoethoxy]-1,1′-biphenyl-4-yl}-1H-pyrazole-3-carboxamide;
4-[(aminocarbonyl)amino]-1-{3′-[2-(diethylamino)-2-oxoethoxy]-1,1′-biphenyl-4-yl}-1H-pyrazole-3-carboxamide;
4-{3-(aminocarbonyl)-4-[(aminocarbonyl)amino]-1H-pyrazol-1-yl}phenylboronic acid;
4-[(aminocarbonyl)amino]-1-[3′-(3-amino-3-oxopropyl)-1,1′-biphenyl-4-yl]-1H-pyrazole-3-carboxamide;
4-[(aminocarbonyl)amino]-1-(3′-cyano-1,1′-biphenyl-3-yl)-1H-pyrazole-3-carboxamide;
4-[(aminocarbonyl)amino]-1-(3′-methoxy-1,1′-biphenyl-3-yl)-1H-pyrazole-3-carboxamide;
4-[(aminocarbonyl)amino]-1-(4′-ethoxy-1,1′-biphenyl-3-yl)-1H-pyrazole-3-carboxamide;
4-[(aminocarbonyl)amino]-1-[4′-(methylthio)-1,1′-biphenyl-3-yl]-1H-pyrazole-3-carboxamide;
4-[(aminocarbonyl)amino]-1-(3′-fluoro-4′-methoxy-1,1′-biphenyl-3-yl)-1H-pyrazole-3-carboxamide;
4-[(aminocarbonyl)amino]-1-(1,1′:2′,1″-terphenyl-3-yl)-1H-pyrazole-3-carboxamide;
4-[(aminocarbonyl)amino]-1-[4′-(methylsulfonyl)-1,1′-biphenyl-3-yl]-1H-pyrazole-3-carboxamide;
1-(1-(4-(4-fluorophenylthio)-2-chlorophenyl)-3-carbamoyl-1H-pyrazol-4-yl)urea;
1-(1-(4-(4-fluorophenylsulfonyl)-2-chlorophenyl)-3-carbamoyl-1H-pyrazol-4-yl)urea;
1-(1-(4-(4-fluorophenylsulfonyl)-2-fluorophenyl)-3-carbamoyl-1H-pyrazol-4-yl)urea;
3-(4′-{3-(aminocarbonyl)-4-[(aminocarbonyl)amino]-1H-pyrazol-1-yl}-1,1′-biphenyl-2-yl)propanoic acid;
4-[(aminocarbonyl)amino]-1-(3′-{3-[(2,4-dimethoxybenzyl)amino]-3-oxopropyl}-1,1′-biphenyl-4-yl)-1H-pyrazole-3-carboxamide;
4-[(aminocarbonyl)amino]-1-{3′-[3-(methylamino)-3-oxopropyl]-1,1′-biphenyl-4-yl}-1H-pyrazole-3-carboxamide;
4-[(aminocarbonyl)amino]-1-{3′-[3-(diethylamino)-3-oxopropyl]-1,1′-biphenyl-4-yl}-1H-pyrazole-3-carboxamide; and
4-[(aminocarbonyl)amino]-1-{3′-[2-(methylamino)-2-oxoethoxy]-1,1′-biphenyl-4-yl}-1H-pyrazole-3-carboxamide.

In a still more particularly preferred embodiment, the compound of Formula I is selected from the group of compounds consisting of:
4-[(aminocarbonyl)amino]-1-[4-bromo-3-(trifluoromethyl)phenyl]-1H-pyrazole-3-carboxamide;
4-[(aminocarbonyl)amino]-1-(3-chloro-4-iodophenyl)-1H-pyrazole-3-carboxamide;
4-[(aminocarbonyl)amino]-1-(2-chloro-3′-hydroxy-1,1′-biphenyl-4-yl)-1H-pyrazole-3-carboxamide;
4-[(aminocarbonyl)amino]-1-(4-iodo-3-methylphenyl)-1H-pyrazole-3-carboxamide;
4-[(aminocarbonyl)amino]-1-[3-(2-chloropyridin-4-yl)phenyl]-1H-pyrazole-3-carboxamide;
4-[(aminocarbonyl)amino]-1-(4-methylphenyl)-1H-pyrazole-3-carboxamide;
4-[(aminocarbonyl)amino]-1-(4-bromophenyl)-1H-pyrazole-3-carboxamide;
4-[(aminocarbonyl)amino]-1-(3-bromophenyl)-1H-pyrazole-3-carboxamide;
4-[(aminocarbonyl)amino]-1-[4-(1H-pyrrol-2-yl)phenyl]-1H-pyrazole-3-carboxamide;
methyl 2-amino-4′-{3-(aminocarbonyl)-4-[(aminocarbonyl)amino]-1H-pyrazol-1-yl}-1,1′-biphenyl-4-carboxylate;
4-[(aminocarbonyl)amino]-1-{4′-[(pyridin-2-ylamino)carbonyl]-1,1′-biphenyl-4-yl}-1H-pyrazole-3-carboxamide;
4-[(aminocarbonyl)amino]-1-[4′-(cyanomethyl)-1,1′-biphenyl-4-yl]-1H-pyrazole-3-carboxamide;
4-[(aminocarbonyl)amino]-1-[4′-(hydroxymethyl)-1,1′-biphenyl-4-yl]-1H-pyrazole-3-carboxamide;
4-[(aminocarbonyl)amino]-1-(4-pyridin-4-ylphenyl)-1H-pyrazole-3-carboxamide;
4-[(aminocarbonyl)amino]-1-(3-pyridin-4-ylphenyl)-1H-pyrazole-3-carboxamide;
4-[(aminocarbonyl)amino]-1-(4′-hydroxy-1,1′-biphenyl-3-yl)-1H-pyrazole-3-carboxamide;
4-[(aminocarbonyl)amino]-1-[3-(1H-pyrazol-4-yl)phenyl]-1H-pyrazole-3-carboxamide;
4-[(aminocarbonyl)amino]-1-[3-(2-methylpyridin-4-yl)phenyl]-1H-pyrazole-3-carboxamide;
4-[(aminocarbonyl)amino]-1-[3-(1H-pyrrol-2-yl)phenyl]-1H-pyrazole-3-carboxamide;
4-[(aminocarbonyl)amino]-1-[3-(3-methylpyridin-4-yl)phenyl]-1H-pyrazole-3-carboxamide;
4-[(aminocarbonyl)amino]-1-[3-(3,5-dimethylisoxazol-4-yl)phenyl]-1H-pyrazole-3-carboxamide;
4-[(aminocarbonyl)amino]-1-(3′-hydroxy-1,1′-biphenyl-3-yl)-1H-pyrazole-3-carboxamide;
4-[(aminocarbonyl)amino]-1-{4′-[(methylamino)carbonyl]-1,1′-biphenyl-3-yl}-1H-pyrazole-3-carboxamide;
4-[(aminocarbonyl)amino]-1-{4′-[(propylamino)carbonyl]-1,1′-biphenyl-3-yl}-1H-pyrazole-3-carboxamide;
1-[3′-(acetylamino)-1,1′-biphenyl-3-yl]-4-[(aminocarbonyl)amino]-1H-pyrazole-3-carboxamide;
4-[(aminocarbonyl)amino]-1-(3′-amino-4′-methyl-1,1′-biphenyl-3-yl)-1H-pyrazole-3-carboxamide;
4-[(aminocarbonyl)amino]-1-[3′-(aminomethyl)-1,1′-biphenyl-3-yl]-1H-pyrazole-3-carboxamide;
4-[(aminocarbonyl)amino]-1-{4′-[(cyclopropylamino)carbonyl]-1,1′-biphenyl-3-yl}-1H-pyrazole-3-carboxamide;
N-[(3′-{3-(aminocarbonyl)-4-[(aminocarbonyl)amino]-1H-pyrazol-1-yl}-1,1′-biphenyl-4-yl)carbonyl]glycine;
4-[(aminocarbonyl)amino]-1-(4′-hydroxy-2′-methyl-1,1′-biphenyl-3-yl)-1H-pyrazole-3-carboxamide;
4-[(aminocarbonyl)amino]-1-[2′-(cyanomethoxy)-5′-fluoro-11′-biphenyl-3-yl]-1H-pyrazole-3-carboxamide;
4′-{3-(aminocarbonyl)-4-[(aminocarbonyl)amino]-1H-pyrazol-1-yl}-3-fluoro-1,1′-biphenyl-4-carboxylic acid;
4-[(aminocarbonyl)amino]-1-(5′-fluoro-2′-hydroxy-1,1′-biphenyl-4-yl)-1H-pyrazole-3-carboxamide;
4-[(aminocarbonyl)amino]-1-(3′-cyano-1,1′-biphenyl-4-yl)-1H-pyrazole-3-carboxamide;
4-[(aminocarbonyl)amino]-1-[2′-(hydroxymethyl)-1,1′-biphenyl-4-yl]-1H-pyrazole-3-carboxamide;
4-[(aminocarbonyl)amino]-1-(3′-methoxy-1,1′-biphenyl-4-yl)-1H-pyrazole-3-carboxamide;
4-[(aminocarbonyl)amino]-1-(4′-fluoro-2′-methyl-1,1′-biphenyl-4-yl)-1H-pyrazole-3-carboxamide;
4-[(aminocarbonyl)amino]-1-(2′-chloro-1,1′-biphenyl-4-yl)-1H-pyrazole-3-carboxamide;
4-[(aminocarbonyl)amino]-1-(2′,4′-difluoro-1,1′-biphenyl-4-yl)-1H-pyrazole-3-carboxamide;
4-[(aminocarbonyl)amino]-1-[4′-(methylthio)-1,1′-biphenyl-4-yl]-1H-pyrazole-3-carboxamide;
4-[(aminocarbonyl)amino]-1-(2′-fluoro-3′-methoxy-1,1′-biphenyl-4-yl)-1H-pyrazole-3-carboxamide;
4-[(aminocarbonyl)amino]-1-(5′-fluoro-2′-methoxy-1,1′-biphenyl-4-yl)-1H-pyrazole-3-carboxamide;
4′-{3-(aminocarbonyl)-4-[(aminocarbonyl)amino]-1H-pyrazol-1-yl}-1,1′-biphenyl-4-carboxylic acid;
4-[(aminocarbonyl)amino]-1-{4′-[(methylamino)carbonyl]-1,1′-biphenyl-4-yl}-1H-pyrazole-3-carboxamide;
4-[(aminocarbonyl)amino]-1-[4′-(aminocarbonyl)-1,1′-biphenyl-4-yl]-1H-pyrazole-3-carboxamide;
4-[(aminocarbonyl)amino]-1-(3′-fluoro-4′-{[(4-methoxybenzyl)amino]carbonyl}-1,1′-biphenyl-4-yl)-1H-pyrazole-3-carboxamide;
4-[(aminocarbonyl)amino]-1-{4′-[(benzylamino)carbonyl]-3′-fluoro-1,1′-biphenyl-4-yl}-1H-pyrazole-3-carboxamide;
4-[(aminocarbonyl)amino]-1-{3′-fluoro-4′-[(methylamino)carbonyl]-1,1′-biphenyl-4-yl}-1H-pyrazole-3-carboxamide;
4-[(aminocarbonyl)amino]-1-(3′-fluoro-4′-{[(3-methoxybenzyl)amino]carbonyl}-1,1′-biphenyl-4-yl)-1H-pyrazole-3-carboxamide;
4-[(aminocarbonyl)amino]-1-(3′-fluoro-4′-{[(2-methoxybenzyl)amino]carbonyl}-1,1′-biphenyl-4-yl)-1H-pyrazole-3-carboxamide;
4-[(aminocarbonyl)amino]-1-(3′-fluoro-4′-{[(4-fluorobenzyl)amino]carbonyl}-1,1′-biphenyl-4-yl)-1H-pyrazole-3-carboxamide;
4-[(aminocarbonyl)amino]-1-(3′-fluoro-4′-{[(3-fluorobenzyl)amino]carbonyl}-1,1′-biphenyl-4-yl)-1H-pyrazole-3-carboxamide;
4-[(aminocarbonyl)amino]-1-[3′-(cyanomethoxy)-1,1′-biphenyl-4-yl]-1H-pyrazole-3-carboxamide;
4-[(aminocarbonyl)amino]-1-[2′-(cyanomethoxy)-1,1′-biphenyl-4-yl]-1H-pyrazole-3-carboxamide;
4-[(aminocarbonyl)amino]-1-[2′-(cyanomethoxy)-5′-fluoro-1,1′-biphenyl-4-yl]-1H-pyrazole-3-carboxamide;
4-[(aminocarbonyl)amino]-1-[2′-(cyanomethoxy)-4′-fluoro-1,1′-biphenyl-4-yl]-1H-pyrazole-3-carboxamide;
4-[(aminocarbonyl)amino]-1-[3′-(2-amino-2-oxoethoxy)-1,1′-biphenyl-4-yl]-1H-pyrazole-3-carboxamide;
4-[(aminocarbonyl)amino]-1-[2′-(cyanomethoxy)-1,1′-biphenyl-3-yl]-1H-pyrazole-3-carboxamide;
4-[(aminocarbonyl)amino]-1-[3′-(cyanomethoxy)-3-fluoro-1,1′-biphenyl-4-yl]-1H-pyrazole-3-carboxamide;
4-[(aminocarbonyl)amino]-1-[2′-(1-cyanoethoxy)-5′-fluoro-1,1′-biphenyl-4-yl]-1H-pyrazole-3-carboxamide;
4-[(aminocarbonyl)amino]-1-{3′-[2-(dimethylamino)-2-oxoethoxy]-1,1′-biphenyl-4-yl}-1H-pyrazole-3-carboxamide;
4-[(aminocarbonyl)amino]-1-[4′-(aminocarbonyl)-3′-fluoro-1,1′-biphenyl-4-yl]-1H-pyrazole-3-carboxamide;
4-[(aminocarbonyl)amino]-1-[3′-(2-cyanoethyl)-1,1′-biphenyl-4-yl]-1H-pyrazole-3-carboxamide;
4-[(aminocarbonyl)amino]-1-[4′-(1H-tetrazol-5-yl)-1,1′-biphenyl-4-yl]-1H-pyrazole-3-carboxamide;
4-[(aminocarbonyl)amino]-1-(3,4-dichlorophenyl)-1H-pyrazole-3-carboxamide;
1-(1-(4-(2-fluorophenylthio)phenyl)-3-carbamoyl-1H-pyrazol-4-yl)urea;
1-(3-carbamoyl-1-(4-(cyclohexylsulfonyl)phenyl)-1H-pyrazol-4-yl)urea;
1-(1-(4-(2-fluorophenylsulfonyl)phenyl)-3-carbamoyl-1H-pyrazol-4-yl)urea;
1-(3-carbamoyl-1-(3-iodophenyl)-1H-pyrazol-4-yl)urea;
1-(1-(4-(4-fluorophenylthio)phenyl)-3-carbamoyl-1H-pyrazol-4-yl)urea;
1-(1-(4-(3-fluorophenylthio)phenyl)-3-carbamoyl-1H-pyrazol-4-yl)urea;
1-(1-(4-(4-fluorophenylsulfinyl)phenyl)-3-carbamoyl-1H-pyrazol-4-yl)urea;
1-(1-(4-(3-fluorophenylsulfinyl)phenyl)-3-carbamoyl-1H-pyrazol-4-yl)urea;
1-(1-(4-(3-fluorophenylsulfonyl)phenyl)-3-carbamoyl-1H-pyrazol-4-yl)urea;
1-(1-(4-(4-fluorophenylsulfonyl)phenyl)-3-carbamoyl-1H-pyrazol-4-yl)urea;
1-(3-carbamoyl-1-(4-(pyridin-4-ylthio)phenyl)-1H-pyrazol-4-yl)urea;
1-(1-(4-(2-hydroxyphenylthio)phenyl)-3-carbamoyl-1H-pyrazol-4-yl)urea;
1-(1-(4-(2-fluorophenylthio)-2-fluorophenyl)-3-carbamoyl-1H-pyrazol-4-yl)urea;
4-[(aminocarbonyl)amino]-1-(4-fluorophenyl)-1H-pyrazole-3-carboxamide;
4-[(aminocarbonyl)amino]-1-(4-chlorophenyl)-1H-pyrazole-3-carboxamide;
4-[(aminocarbonyl)amino]-1-(4-iodophenyl)-1H-pyrazole-3-carboxamide;
4-[(aminocarbonyl)amino]-1-(4-isopropylphenyl)-1H-pyrazole-3-carboxamide;
4-[(aminocarbonyl)amino]-1-(1H-indol-5-yl)-1H-pyrazole-3-carboxamide;
4-[(aminocarbonyl)amino]-1-(2-naphthyl)-1H-pyrazole-3-carboxamide;
4-[(aminocarbonyl)amino]-1-[2-chloro-2′-(cyanomethoxy)-1,1′-biphenyl-4-yl]-1H-pyrazole-3-carboxamide;
4-[(aminocarbonyl)amino]-1-(4-bromo-3-fluorophenyl)-1H-pyrazole-3-carboxamide;
4-[(aminocarbonyl)amino]-1-[3′-hydroxy-2-(trifluoromethyl)-1,1′-biphenyl-4-yl]-1H-pyrazole-3-carboxamide;
1-(3′-hydroxy-2-methyl-biphenyl-4-yl)-4-ureido-1H-pyrazole-3-carboxylic acid amide;
1-(2′-Cyanomethoxy-4′-fluoro-2-methyl-biphenyl-4-yl)-4-ureido-1H-pyrazole-3-carboxylic acid amide;
4-[(aminocarbonyl)amino]-1-(3-fluoro-3′-hydroxy-1,1′-biphenyl-4-yl)-1H-pyrazole-3-carboxamide;
4′-{3-(aminocarbonyl)-4-[(aminocarbonyl)amino]-1H-pyrazol-1-yl}-3,3′-difluoro-1,1′-biphenyl-4-carboxylic acid;
4-[(aminocarbonyl)amino]-1-(3-fluoro-2′-hydroxy-1,1′-biphenyl-4-yl)-1H-pyrazole-3-carboxamide;
4-[(aminocarbonyl)amino]-1-[2′-(cyanomethoxy)-3-fluoro-1,1′-biphenyl-4-yl]-1H-pyrazole-3-carboxamide;
4-[(aminocarbonyl)amino]-1-(2-chloro-4-iodophenyl)-1H-pyrazole-3-carboxamide;
4-[(aminocarbonyl)amino]-1-[2′-(2-amino-2-oxoethoxy)-5′-fluoro-1,1′-biphenyl-4-yl]-1H-pyrazole-3-carboxamide;
4-[(aminocarbonyl)amino]-1-[3′-(1,3-oxazol-2-ylmethoxy)-1,1′-biphenyl-4-yl]-1H-pyrazole-3-carboxamide;
4-[(aminocarbonyl)amino]-1-[5′-fluoro-2′-(1,3-oxazol-2-ylmethoxy)-1,1′-biphenyl-4-yl]-1H-pyrazole-3-carboxamide;
4-[(aminocarbonyl)amino]-1-[4-(4,4,5,5-tetramethyl-1,3,2-dioxaborolan-2-yl)phenyl]-1H-pyrazole-3-carboxamide;
4-[(aminocarbonyl)amino]-1-(2,4′-difluoro-2′-hydroxy-1,1′-biphenyl-4-yl)-1H-pyrazole-3-carboxamide;
1-(3-carbamoyl-1-(4-(cyclopentylsulfinyl)phenyl)-1H-pyrazol-4-yl)urea;
1-(3-carbamoyl-1-(4-(pyridin-2-ylsulfinyl)phenyl)-1H-pyrazol-4-yl)urea;
1-(1-(4-(2-hydroxyphenylsulfinyl)phenyl)-3-carbamoyl-1H-pyrazol-4-yl)urea;
1-(3-carbamoyl-1-(4-(pyridin-2-ylsulfonyl)phenyl)-1H-pyrazol-4-yl)urea;
1-(3-carbamoyl-1-(4-(cyclopentylsulfonyl)phenyl)-1H-pyrazol-4-yl)urea;
1-(1-(4-(2-hydroxyphenylsulfonyl)phenyl)-3-carbamoyl-1H-pyrazol-4-yl)urea;
1-(2′-carbamoylmethoxy-4′-fluoro-2-methyl-biphenyl-4-yl)-4-ureido-1H-pyrazole-3-carboxylic acid amide;
1-[4-(4-fluoro-benzenesulfonyl)-3-methyl-phenyl]-4-ureido-1H-pyrazole-3-carboxylic acid amide;
1-(3-hydroxy-phenyl)-4-ureido-1H-pyrazole-3-carboxylic acid 4-methoxy-benzylamide;
4-[(aminocarbonyl)amino]-1-[4-(aminosulfonyl)phenyl]-1H-pyrazole-3-carboxamide;
4-[(aminocarbonyl)amino]-1-(4-chloro-3-methylphenyl)-1H-pyrazole-3-carboxamide;
4-[(aminocarbonyl)amino]-1-{3′-[2-(ethylamino)-2-oxoethoxy]-1,1′-biphenyl-4-yl}-1H-pyrazole-3-carboxamide;
4-[(aminocarbonyl)amino]-1-{3′-[2-(diethylamino)-2-oxoethoxy]-1,1′-biphenyl-4-yl}-1H-pyrazole-3-carboxamide;
4-{3-(aminocarbonyl)-4-[(aminocarbonyl)amino]-1H-pyrazol-1-yl}phenylboronic acid;
4-[(aminocarbonyl)amino]-1-[3′-(3-amino-3-oxopropyl)-1,1′-biphenyl-4-yl]-1H-pyrazole-3-carboxamide;
4-[(aminocarbonyl)amino]-1-(3′-cyano-1,1′-biphenyl-3-yl)-1H-pyrazole-3-carboxamide;
4-[(aminocarbonyl)amino]-1-[4′-(methylsulfonyl)-1,1′-biphenyl-3-yl]-1H-pyrazole-3-carboxamide;
1-(1-(4-(4-fluorophenylthio)-2-chlorophenyl)-3-carbamoyl-1H-pyrazol-4-yl)urea;
1-(1-(4-(4-fluorophenylsulfonyl)-2-chlorophenyl)-3-carbamoyl-1H-pyrazol-4-yl)urea;
1-(1-(4-(4-fluorophenylsulfonyl)-2-fluorophenyl)-3-carbamoyl-1H-pyrazol-4-yl)urea;
4-[(aminocarbonyl)amino]-1-{3′-[3-(methylamino)-3-oxopropyl]-1,1′-biphenyl-4-yl}-1H-pyrazole-3-carboxamide;
4-[(aminocarbonyl)amino]-1-{3′-[3-(diethylamino)-3-oxopropyl]-1,1′-biphenyl-4-yl}-1H-pyrazole-3-carboxamide; and
4-[(aminocarbonyl)amino]-1-{3′-[2-(methylamino)-2-oxoethoxy]-1,1′-biphenyl-4-yl}-1H-pyrazole-3-carboxamide.

In another particularly preferred embodiment, the compound of Formula I is selected from the group of compounds consisting of:
4-[(aminocarbonyl)amino]-1-[4-bromo-3-(trifluoromethyl)phenyl]-1H-pyrazole-3-carboxamide;
4-[(aminocarbonyl)amino]-1-(4-bromo-3-ethoxyphenyl)-1H-pyrazole-3-carboxamide;
4-[(aminocarbonyl)amino]-1-(4-iodo-2-methylphenyl)-1H-pyrazole-3-carboxamide;
4-[(aminocarbonyl)amino]-1-(3′-hydroxy-3-methyl-1,1′-biphenyl-4-yl)-1H-pyrazole-3-carboxamide;
4-[(aminocarbonyl)amino]-1-(2-chloro-3′-hydroxy-1,1′-biphenyl-4-yl)-1H-pyrazole-3-carboxamide;
4-[(aminocarbonyl)amino]-1-(4-iodo-3-methylphenyl)-1H-pyrazole-3-carboxamide;
1-(3-bromophenyl)-4-{[(methylamino)carbonyl]amino}-1H-pyrazole-3-carboxamide;
1-(4-bromophenyl)-4-{[(methylamino)carbonyl]amino}-1H-pyrazole-3-carboxamide;
4-[(aminocarbonyl)amino]-1-(4-bromophenyl)-1H-pyrazole-3-carboxamide;
4-[(aminocarbonyl)amino]-1-(4′-fluoro-2′-hydroxy-1,1′-biphenyl-3-yl)-1H-pyrazole-3-carboxamide;
4-[(aminocarbonyl)amino]-1-(5′-fluoro-2′-hydroxy-1,1′-biphenyl-3-yl)-1H-pyrazole-3-carboxamide;
4-[(aminocarbonyl)amino]-1-[2′-(cyanomethoxy)-5′-fluoro-1,1′-biphenyl-3-yl]-1H-pyrazole-3-carboxamide;
4′-{3-(aminocarbonyl)-4-[(aminocarbonyl)amino]-1H-pyrazol-1-yl}-3-fluoro-1,1′-biphenyl-4-carboxylic acid;
4-[(aminocarbonyl)amino]-1-(4′-fluoro-2′-hydroxy-1,1′-biphenyl-4-yl)-1H-pyrazole-3-carboxamide;
4-[(aminocarbonyl)amino]-1-(5′-fluoro-2′-hydroxy-1,1′-biphenyl-4-yl)-1H-pyrazole-3-carboxamide;
4-[(aminocarbonyl)amino]-1-(4′-cyano-1,1′-biphenyl-4-yl)-1H-pyrazole-3-carboxamide;
4-[(aminocarbonyl)amino]-1-(1,1′-biphenyl-4-yl)-1H-pyrazole-3-carboxamide;
4-[(aminocarbonyl)amino]-1-[4-(1H-indol-2-yl)phenyl]-1H-pyrazole-3-carboxamide;
4-[(aminocarbonyl)amino]-1-(4′-{[[2-(dimethylamino)ethyl](methyl)amino]carbonyl}-3′-fluoro-1,1′-biphenyl-4-yl)-1H-pyrazole-3-carboxamide;
4-fluorobenzyl 4′-{3-(aminocarbonyl)-4-[(aminocarbonyl)amino]-1H-pyrazol-1-yl}-3-fluoro-1,1′-biphenyl-4-carboxylate;
4-[(aminocarbonyl)amino]-1-[2′-(cyanomethoxy)-5′-fluoro-1,1′-biphenyl-4-yl]-1H-pyrazole-3-carboxamide;
4-[(aminocarbonyl)amino]-1-[2′-(cyanomethoxy)-4′-fluoro-1,1′-biphenyl-4-yl]-1H-pyrazole-3-carboxamide;
tert-butyl [(4′-{3-(aminocarbonyl)-4-[(aminocarbonyl)amino]-1H-pyrazol-1-yl}-1,1′-biphenyl-3-yl)oxy]acetate;
4-[(aminocarbonyl)amino]-1-[3′-(2-amino-2-oxoethoxy)-1,1′-biphenyl-4-yl]-1H-pyrazole-3-carboxamide;
4-[(aminocarbonyl)amino]-1-[3′-(2-cyanoethyl)-1,1′-biphenyl-4-yl]-1H-pyrazole-3-carboxamide;
4-[(aminocarbonyl)amino]-1-[4′-(1H-tetrazol-5-yl)-1,1′-biphenyl-4-yl]-1H-pyrazole-3-carboxamide;
1-(3-carbamoyl-1-(4-(cyclohexylsulfonyl)phenyl)-1H-pyrazol-4-yl)urea;
1-(1-(4-(2-fluorophenylsulfonyl)phenyl)-3-carbamoyl-1H-pyrazol-4-yl)urea;
1-(1-(4-(4-fluorophenylsulfonyl)phenyl)-3-carbamoyl-1H-pyrazol-4-yl)urea;
1-(3-carbamoyl-1-(4-(pyridin-2-ylthio)phenyl)-1H-pyrazol-4-yl)urea;
4-[(Aminocarbonyl)amino]-1-(3-chloro-4-methoxyphenyl)-1H-pyrazole-3-carboxamide;
4-[(aminocarbonyl)amino]-1-(4-iodophenyl)-1H-pyrazole-3-carboxamide;
4-[(aminocarbonyl)amino]-1-(4-bromo-3-fluorophenyl)-1H-pyrazole-3-carboxamide;
4-[(aminocarbonyl)amino]-1-(3-fluoro-3′-hydroxy-1,1′-biphenyl-4-yl)-1H-pyrazole-3-carboxamide;
4-[(aminocarbonyl)amino]-1-(2,5′-difluoro-2′-hydroxy-1,1′-biphenyl-4-yl)-1H-pyrazole-3-carboxamide;
4-[(aminocarbonyl)amino]-1-(2-fluoro-4′-hydroxy-1,1′-biphenyl-4-yl)-1H-pyrazole-3-carboxamide;
4-[(aminocarbonyl)amino]-1-(2,3′-difluoro-4′-hydroxy-1,1′-biphenyl-4-yl)-1H-pyrazole-3-carboxamide;
4-[(aminocarbonyl)amino]-1-[2′-(2-amino-2-oxoethoxy)-2-fluoro-1,1′-biphenyl-4-yl]-1H-pyrazole-3-carboxamide;
4-[(aminocarbonyl)amino]-1-(2-fluoro-2′-methoxy-1,1′-biphenyl-4-yl)-1H-pyrazole-3-carboxamide;
4-[(aminocarbonyl)amino]-1-[2′-(2-amino-2-oxoethoxy)-2,4′-difluoro-1,1′-biphenyl-4-yl]-1H-pyrazole-3-carboxamide;
4-[(aminocarbonyl)amino]-1-(2,5′-difluoro-2′-methoxy-1,1′-biphenyl-4-yl)-1H-pyrazole-3-carboxamide;
4-[(aminocarbonyl)amino]-1-{3-chloro-4-[(2-fluorophenyl)sulfonyl]phenyl}-1H-pyrazole-3-carboxamide;
4-[(aminocarbonyl)amino]-1-(2-chloro-4′-hydroxy-1,1′-biphenyl-4-yl)-1H-pyrazole-3-carboxamide;
1-(3-chloro-2-fluoro-phenyl)-4-ureido-1 h-pyrazole-3-carboxylic acid amide;
4-[(aminocarbonyl)amino]-1-(4-bromo-3-hydroxyphenyl)-1H-pyrazole-3-carboxamide;
1-(1-(4-(m-tolylthio)phenyl)-3-carbamoyl-1H-pyrazol-4-yl)urea;
1-(1-(4-(3-bromophenylthio)phenyl)-3-carbamoyl-1H-pyrazol-4-yl)urea;
1-(3-carbamoyl-1-(4-(pyridin-4-ylsulfinyl)phenyl)-1H-pyrazol-4-yl)urea;
1-(3-carbamoyl-1-(4-(pyridin-4-ylsulfonyl)phenyl)-1H-pyrazol-4-yl)urea;
1-(3-hydroxy-phenyl)-4-ureido-1H-pyrazole-3-carboxylic acid amide;
4-[(aminocarbonyl)amino]-1-(4-fluoro-3-methylphenyl)-1H-pyrazole-3-carboxamide;
4-[(aminocarbonyl)amino]-1-(4-propylphenyl)-1H-pyrazole-3-carboxamide;
4-[(aminocarbonyl)amino]-1-(3-methyl-4-propoxyphenyl)-1H-pyrazole-3-carboxamide;
4-[(aminocarbonyl)amino]-1-[4-(benzyloxy)-3-methylphenyl]-1H-pyrazole-3-carboxamide;
1-(4′-hydroxy-2-methyl-biphenyl-4-yl)-4-ureido-1H-pyrazole-3-carboxylic acid amide;
1-(4′-hydroxy-2,2′-dimethyl-biphenyl-4-yl)-4-ureido-1H-pyrazole-3-carboxylic acid amide;
1-(3′-fluoro-4′-hydroxy-2-methyl-biphenyl-4-yl)-4-ureido-1H-pyrazole-3-carboxylic acid amide;
1-[4-(3-fluoro-phenylsulfanyl)-3-methyl-phenyl]-4-ureido-1H-pyrazole-3-carboxylic acid amide;
1-[4-(3-fluoro-benzenesulfonyl)-3-methyl-phenyl]-4-ureido-1H-pyrazole-3-carboxylic acid amide;
1-m-tolyl-4-ureido-1H-pyrazole-3-carboxylic acid amide;
1-(3-fluoro-phenyl)-4-ureido-1H-pyrazole-3-carboxylic acid amide;
1-(3-trifluoromethyl-phenyl)-4-ureido-1H-pyrazole-3-carboxylic acid amide;
1-o-tolyl-4-ureido-1H-pyrazole-3-carboxylic acid amide;
4-[(aminocarbonyl)amino]-1-(3-fluoro-4-methylphenyl)-1H-pyrazole-3-carboxamide;
4-[(aminocarbonyl)amino]-1-[4-(trifluoromethoxy)phenyl]-1H-pyrazole-3-carboxamide;
4-[(aminocarbonyl)amino]-1-(3,4-dimethoxyphenyl)-1H-pyrazole-3-carboxamide;
4-[(aminocarbonyl)amino]-1-(4-propoxyphenyl)-1H-pyrazole-3-carboxamide; and
4-[(aminocarbonyl)amino]-1-(3-chloro-4-propoxyphenyl)-1H-pyrazole-3-carboxamide.

In another still more particularly preferred embodiment, the compound of Formula I is selected from the group of compounds consisting of:
4-[(aminocarbonyl)amino]-1-[4-bromo-3-(trifluoromethyl)phenyl]-1H-pyrazole-3-carboxamide;
4-[(aminocarbonyl)amino]-1-(4-bromo-3-ethoxyphenyl)-1H-pyrazole-3-carboxamide;
4-[(aminocarbonyl)amino]-1-(2-chloro-3′-hydroxy-1,1′-biphenyl-4-yl)-1H-pyrazole-3-carboxamide;
4-[(aminocarbonyl)amino]-1-(4-iodo-3-methylphenyl)-1H-pyrazole-3-carboxamide;
4-[(aminocarbonyl)amino]-1-(4-bromophenyl)-1H-pyrazole-3-carboxamide;
4-[(aminocarbonyl)amino]-1-[2′-(cyanomethoxy)-5′-fluoro-1,1′-biphenyl-3-yl]-1H-pyrazole-3-carboxamide;
4′-{3-(aminocarbonyl)-4-[(aminocarbonyl)amino]-1H-pyrazol-1-yl}-3-fluoro-1,1′-biphenyl-4-carboxylic acid;
4-[(aminocarbonyl)amino]-1-(4′-fluoro-2′-hydroxy-1,1′-biphenyl-4-yl)-1H-pyrazole-3-carboxamide;
4-[(aminocarbonyl)amino]-1-(5′-fluoro-2′-hydroxy-1,1′-biphenyl-4-yl)-1H-pyrazole-3-carboxamide;
4-[(aminocarbonyl)amino]-1-(4′-cyano-1,1′-biphenyl-4-yl)-1H-pyrazole-3-carboxamide;
4-[(aminocarbonyl)amino]-1-[2′-(cyanomethoxy)-5′-fluoro-1,1′-biphenyl-4-yl]-1H-pyrazole-3-carboxamide;
4-[(aminocarbonyl)amino]-1-[2′-(cyanomethoxy)-4′-fluoro-1,1′-biphenyl-4-yl]-1H-pyrazole-3-carboxamide;
4-[(aminocarbonyl)amino]-1-[3′-(2-amino-2-oxoethoxy)-1,1′-biphenyl-4-yl]-1H-pyrazole-3-carboxamide;
4-[(aminocarbonyl)amino]-1-[3′-(2-cyanoethyl)-1,1′-biphenyl-4-yl]-1H-pyrazole-3-carboxamide;
4-[(aminocarbonyl)amino]-1-[4′-(1H-tetrazol-5-yl)-1,1′-biphenyl-4-yl]-1H-pyrazole-3-carboxamide;
1-(3-carbamoyl-1-(4-(cyclohexylsulfonyl)phenyl)-1H-pyrazol-4-yl)urea;
1-(1-(4-(2-fluorophenylsulfonyl)phenyl)-3-carbamoyl-1H-pyrazol-4-yl)urea;
1-(1-(4-(4-fluorophenylsulfonyl)phenyl)-3-carbamoyl-1H-pyrazol-4-yl)urea;
1-(3-carbamoyl-1-(4-(pyridin-2-ylthio)phenyl)-1H-pyrazol-4-yl)urea;
4-[(aminocarbonyl)amino]-1-(4-iodophenyl)-1H-pyrazole-3-carboxamide;
4-[(aminocarbonyl)amino]-1-(4-bromo-3-fluorophenyl)-1H-pyrazole-3-carboxamide;
4-[(aminocarbonyl)amino]-1-(3-fluoro-3′-hydroxy-1,1′-biphenyl-4-yl)-1H-pyrazole-3-carboxamide;
4-[(aminocarbonyl)amino]-1-(2,5′-difluoro-2′-hydroxy-1,1′-biphenyl-4-yl)-1H-pyrazole-3-carboxamide;
4-[(aminocarbonyl)amino]-1-(2-fluoro-4′-hydroxy-1,1′-biphenyl-4-yl)-1H-pyrazole-3-carboxamide;
4-[(aminocarbonyl)amino]-1-[2′-(2-amino-2-oxoethoxy)-2-fluoro-1,1′-biphenyl-4-yl]-1H-pyrazole-3-carboxamide;
4-[(aminocarbonyl)amino]-1-[2′-(2-amino-2-oxoethoxy)-2,4′-difluoro-1,1′-biphenyl-4-yl]-1H-pyrazole-3-carboxamide;
4-[(aminocarbonyl)amino]-1-(2,5′-difluoro-2′-methoxy-1,1′-biphenyl-4-yl)-1H-pyrazole-3-carboxamide;
4-[(aminocarbonyl)amino]-1-{3-chloro-4-[(2-fluorophenyl)sulfonyl]phenyl}-1H-pyrazole-3-carboxamide;
4-[(aminocarbonyl)amino]-1-(2-chloro-4′-hydroxy-1,1′-biphenyl-4-yl)-1H-pyrazole-3-carboxamide;
1-(3-chloro-2-fluoro-phenyl)-4-ureido-1 h-pyrazole-3-carboxylic acid amide;
4-[(aminocarbonyl)amino]-1-(4-bromo-3-hydroxyphenyl)-1H-pyrazole-3-carboxamide;
1-(3-carbamoyl-1-(4-(pyridin-4-ylsulfinyl)phenyl)-1H-pyrazol-4-yl)urea;
1-(3-carbamoyl-1-(4-(pyridin-4-ylsulfonyl)phenyl)-1H-pyrazol-4-yl)urea;
1-(3-hydroxy-phenyl)-4-ureido-1H-pyrazole-3-carboxylic acid amide;
4-[(aminocarbonyl)amino]-1-(4-fluoro-3-methylphenyl)-1H-pyrazole-3-carboxamide;
4-[(aminocarbonyl)amino]-1-(4-propylphenyl)-1H-pyrazole-3-carboxamide;
4-[(aminocarbonyl)amino]-1-(3-methyl-4-propoxyphenyl)-1H-pyrazole-3-carboxamide;
1-(4′-hydroxy-2-methyl-biphenyl-4-yl)-4-ureido-1H-pyrazole-3-carboxylic acid amide;
1-(4′-hydroxy-2,2′-dimethyl-biphenyl-4-yl)-4-ureido-1H-pyrazole-3-carboxylic acid amide;
1-(3′-fluoro-4′-hydroxy-2-methyl-biphenyl-4-yl)-4-ureido-1H-pyrazole-3-carboxylic acid amide;
1-[4-(3-fluoro-benzenesulfonyl)-3-methyl-phenyl]-4-ureido-1H-pyrazole-3-carboxylic acid amide;
1-m-tolyl-4-ureido-1H-pyrazole-3-carboxylic acid amide;
1-(3-fluoro-phenyl)-4-ureido-1H-pyrazole-3-carboxylic acid amide;
4-[(aminocarbonyl)amino]-1-(3-fluoro-4-methylphenyl)-1H-pyrazole-3-carboxamide; and
4-[(aminocarbonyl)amino]-1-[4-(trifluoromethoxy)phenyl]-1H-pyrazole-3-carboxamide.

In another particularly preferred embodiment, the compound of Formula I is selected from the group of compounds consisting of:
4-[(aminocarbonyl)amino]-1-[4-bromo-3-(trifluoromethyl)phenyl]-1H-pyrazole-3-carboxamide;
4-[(aminocarbonyl)amino]-1-(4-bromo-3-ethoxyphenyl)-1H-pyrazole-3-carboxamide;
4-[(aminocarbonyl)amino]-1-(4-iodo-2-methylphenyl)-1H-pyrazole-3-carboxamide;
4-[(aminocarbonyl)amino]-1-(3′-hydroxy-3-methyl-1,1′-biphenyl-4-yl)-1H-pyrazole-3-carboxamide;
4-[(aminocarbonyl)amino]-1-(2-chloro-3′-hydroxy-1,1′-biphenyl-4-yl)-1H-pyrazole-3-carboxamide;
4-[(aminocarbonyl)amino]-1-(4-iodo-3-methylphenyl)-1H-pyrazole-3-carboxamide;
1-(3-bromophenyl)-4-{[(methylamino)carbonyl]amino}-1H-pyrazole-3-carboxamide;
1-(4-bromophenyl)-4-{[(methylamino)carbonyl]amino}-1H-pyrazole-3-carboxamide;
4-[(aminocarbonyl)amino]-1-(4-bromophenyl)-1H-pyrazole-3-carboxamide;
4-[(aminocarbonyl)amino]-1-(4′-fluoro-2′-hydroxy-1,1′-biphenyl-3-yl)-1H-pyrazole-3-carboxamide;
4-[(aminocarbonyl)amino]-1-(5′-fluoro-2′-hydroxy-1,1′-biphenyl-3-yl)-1H-pyrazole-3-carboxamide;
4-[(aminocarbonyl)amino]-1-[2′-(cyanomethoxy)-5′-fluoro-1,1′-biphenyl-3-yl]-1H-pyrazole-3-carboxamide;
4′-{3-(aminocarbonyl)-4-[(aminocarbonyl)amino]-1H-pyrazol-1-yl}-3-fluoro-1,1′-biphenyl-4-carboxylic acid;
4-[(aminocarbonyl)amino]-1-(4′-fluoro-2′-hydroxy-1,1′-biphenyl-4-yl)-1H-pyrazole-3-carboxamide;
4-[(aminocarbonyl)amino]-1-(5′-fluoro-2′-hydroxy-1,1′-biphenyl-4-yl)-1H-pyrazole-3-carboxamide;
4-[(aminocarbonyl)amino]-1-(4′-cyano-1,1′-biphenyl-4-yl)-1H-pyrazole-3-carboxamide;
4-[(aminocarbonyl)amino]-1-(1,1′-biphenyl-4-yl)-1H-pyrazole-3-carboxamide;
4-[(aminocarbonyl)amino]-1-(4′-{[[2-(dimethylamino)ethyl](methyl)amino]carbonyl}-3′-fluoro-1,1′-biphenyl-4-yl)-1H-pyrazole-3-carboxamide;
4-[(aminocarbonyl)amino]-1-[2′-(cyanomethoxy)-5′-fluoro-1,1′-biphenyl-4-yl]-1H-pyrazole-3-carboxamide;
4-[(aminocarbonyl)amino]-1-[2′-(cyanomethoxy)-4′-fluoro-111′-biphenyl-4-yl]-1H-pyrazole-3-carboxamide;
tert-butyl [(4′-{3-(aminocarbonyl)-4-[(aminocarbonyl)amino]-1H-pyrazol-1-yl}-1,1′-biphenyl-3-yl)oxy]acetate;
4-[(aminocarbonyl)amino]-1-[3′-(2-amino-2-oxoethoxy)-1,1′-biphenyl-4-yl]-1H-pyrazole-3-carboxamide;
4-[(aminocarbonyl)amino]-1-[3′-(2-cyanoethyl)-1,1′-biphenyl-4-yl]-1H-pyrazole-3-carboxamide;
4-[(aminocarbonyl)amino]-1-[4′-(1H-tetrazol-5-yl)-1,1′-biphenyl-4-yl]-1H-pyrazole-3-carboxamide;
1-(3-carbamoyl-1-(4-(cyclohexylsulfonyl)phenyl)-1H-pyrazol-4-yl)urea;
1-(1-(4-(2-fluorophenylsulfonyl)phenyl)-3-carbamoyl-1H-pyrazol-4-yl)urea;
1-(1-(4-(4-fluorophenylsulfonyl)phenyl)-3-carbamoyl-1H-pyrazol-4-yl)urea;
1-(3-carbamoyl-1-(4-(pyridin-2-ylthio)phenyl)-1H-pyrazol-4-yl)urea;
4-[(Aminocarbonyl)amino]-1-(3-chloro-4-methoxyphenyl)-1H-pyrazole-3-carboxamide;
4-[(aminocarbonyl)amino]-1-(4-iodophenyl)-1H-pyrazole-3-carboxamide;
4-[(aminocarbonyl)amino]-1-(4-bromo-3-fluorophenyl)-1H-pyrazole-3-carboxamide; and
4-[(aminocarbonyl)amino]-1-(3-fluoro-3′-hydroxy-1,1′-biphenyl-4-yl)-1H-pyrazole-3-carboxamide.

In another still more particularly preferred embodiment, the compound of Formula I is selected from the group of compounds consisting of:
4-[(aminocarbonyl)amino]-1-[4-bromo-3-(trifluoromethyl)phenyl]-1H-pyrazole-3-carboxamide;
4-[(aminocarbonyl)amino]-1-(2-chloro-3′-hydroxy-1,1′-biphenyl-4-yl)-1H-pyrazole-3-carboxamide;
4-[(aminocarbonyl)amino]-1-(4-iodo-3-methylphenyl)-1H-pyrazole-3-carboxamide;
4-[(aminocarbonyl)amino]-1-(4-bromophenyl)-1H-pyrazole-3-carboxamide;
4-[(aminocarbonyl)amino]-1-[2′-(cyanomethoxy)-5′-fluoro-1,1′-biphenyl-3-yl]-1H-pyrazole-3-carboxamide;
4′-{3-(aminocarbonyl)-4-[(aminocarbonyl)amino]-1H-pyrazol-1-yl}-3-fluoro-1,1′-biphenyl-4-carboxylic acid;
4-[(aminocarbonyl)amino]-1-(5′-fluoro-2′-hydroxy-1,1′-biphenyl-4-yl)-1H-pyrazole-3-carboxamide;
4-[(aminocarbonyl)amino]-1-[2′-(cyanomethoxy)-5′-fluoro-1,1′-biphenyl-4-yl]-1H-pyrazole-3-carboxamide;
4-[(aminocarbonyl)amino]-1-[2′-(cyanomethoxy)-4′-fluoro-1,1′-biphenyl-4-yl]-1H-pyrazole-3-carboxamide;
4-[(aminocarbonyl)amino]-1-[3′-(2-amino-2-oxoethoxy)-1,1′-biphenyl-4-yl]-1H-pyrazole-3-carboxamide;
4-[(aminocarbonyl)amino]-1-[3′-(2-cyanoethyl)-1,1′-biphenyl-4-yl]-1H-pyrazole-3-carboxamide;
4-[(aminocarbonyl)amino]-1-[4′-(1H-tetrazol-5-yl)-1,1′-biphenyl-4-yl]-1H-pyrazole-3-carboxamide;
1-(3-carbamoyl-1-(4-(cyclohexylsulfonyl)phenyl)-1H-pyrazol-4-yl)urea;
1-(1-(4-(2-fluorophenylsulfonyl)phenyl)-3-carbamoyl-1H-pyrazol-4-yl)urea;
1-(1-(4-(4-fluorophenylsulfonyl)phenyl)-3-carbamoyl-1H-pyrazol-4-yl)urea;
4-[(aminocarbonyl)amino]-1-(4-iodophenyl)-1H-pyrazole-3-carboxamide;
4-[(aminocarbonyl)amino]-1-(4-bromo-3-fluorophenyl)-1H-pyrazole-3-carboxamide; and
4-[(aminocarbonyl)amino]-1-(3-fluoro-3′-hydroxy-1,1′-biphenyl-4-yl)-1H-pyrazole-3-carboxamide.
   Definitions

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical.

The term “halo” denotes halogen atoms such as fluorine, chlorine, bromine, or iodine.

The term “carbonyl”, whether used alone or with other terms such as “alkylcarbonyl”, denotes —(C═O)—.

The terms “carboxy” or “carboxyl”, whether used alone or with other terms, such as “carboxyalkyl”, denotes —CO₂H.

The term “sulfonyl,” whether used alone or linked to other terms such as alkylsulfonyl, denotes the divalent radical —SO₂—.

The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-N-hydroxyamidoalkyl”, embraces a carbonyl radical substituted with an amino radical.

The terms “N-alkylamido” and “N,N-dialkylamido” denote amido groups which have been substituted with one alkyl radical and with two alkyl radicals, respectively.

The terms “N-monoarylamido” and “N-alkyl-N-arylamido” denote amido radicals substituted, respectively, with one aryl radical, and one alkyl and one aryl radical.

The term “N-alkyl-N-hydroxyamido” embraces amido radicals substituted with a hydroxyl radical and with an alkyl radical.

The terms “sulfamyl” or “sulfonamidyl” denotes a sulfonyl radical substituted with an amino radical, forming a sulfonamide (—SO₂NH₂). The amino radical may be substituted with alkyl and/or aryl moieties to form, e.g., “N-alkylsulfamyl”, “N-arylsulfamyl”, “N,N-dialkylsulfamyl,” and “N-alkyl-N-arylsulfamyl” radicals.

The term “amidino” denotes a —C(═NH)NH₂ radical.

The term “cyanoamidino” denotes a —C(═N—CN)NH₂ radical.

The term “alkyl,” used alone or within other terms such as “haloalkyl” and “alkylsulfonyl,” embraces linear or branched radicals having one to about twenty carbon atoms. More preferred are “lower alkyl” radicals having one to about eight carbon atoms. Examples of alkyl radicals include methyl, ethyl, propyl (including n-propyl and isopropyl), butyl (including n-butyl, isobutyl, sec-butyl, and t-butyl), pentyl (including n-pentyl and isoamyl), hexyl, octyl and the like.

The term “cycloalkyl” embraces radicals having three to ten carbon atoms, and includes monocyclic, bicyclic, and tricyclic radicals. Examples of cycloalkyl radicals include cyclopropyl, cyclobutyl, cyclopentyl, cyclohexyl, cycloheptyl, decahydronaphthyl, octahydroindyl, octahydropentalene, bicyclo[1.1.0]butyl, bicyclo[2.1.0]pentyl, bicyclo[1.1.1]pentyl, bicyclo[2.1.1]hexyl, bicyclo[2.2.1]heptyl, bicyclo[3.1.1]heptyl, bicyclo[3.2.1]octyl, bicyclo[2.2.2]octyl, and bicyclo[4.2.2]decyl.

The term “alkylcarbonyl” embraces radicals having a carbonyl radical substituted with an alkyl radical. An example of an alkylcarbonyl radical is acetyl.

The term “alkylthio” embraces radicals containing a linear or branched alkyl radical, of one to ten carbon atoms, attached to a divalent sulfur atom. An example of an alkylthio radical is methylthio (CH₃S—).

The term “alkylsulfinyl” embraces radicals containing a linear or branched alkyl radical, of one to ten carbon atoms, attached to a divalent —S(═O)— radical. An example of an alkylsulfinyl radical is methylsulfinyl (CH₃S(═O)—).

The term “alkylsulfonyl” embraces alkyl radicals as defined above attached to a divalent sulfonyl radical, —SO₂—.

The term “amidoalkyl” embraces alkyl radicals substituted with amido radicals.

The term “N-alkyl-N-hydroxyamidoalkyl” embraces alkyl radicals substituted with an N-alkyl-N-hydroxyamido radical.

The term “aminoalkyl” embraces alkyl radicals substituted with amino radicals.

The term “carboxyalkyl” embraces radicals having a carboxyl moiety attached to an alkyl radical.

The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihaloalkyl radicals may have two of the same halo atoms or a combination of different halo radicals; polyhaloalkyl radicals may have more than two of the same halo atoms or a combination of different halo radicals.

The term “hydroxyalkyl” embraces linear or branched alkyl radicals having one to about ten carbon atoms, any of which may be substituted with one or more hydroxyl radicals.

The terms “N-alkylamino” and “N,N-dialkylamino” denote amino groups which have been substituted with one alkyl radical and with two alkyl radicals, respectively.

The term “alkoxy” embraces linear or branched oxy-containing alkyl radicals having one to about ten carbon atoms. Examples of “alkoxy” radicals include methoxy and butoxy.

The term “alkoxyalkyl” embraces linear or branched alkyl radicals having one to about ten carbon atoms substituted by one or more alkoxy radicals each having one to about ten carbon atoms.

“Alkoxy” or “alkoxyalkyl” radicals may be further substituted with one or more halo atoms, such as fluoro, chloro, or bromo, to provide “haloalkoxy” or “haloalkoxyalkyl” radicals.

The term “alkoxycarbonyl” means a radical containing an alkoxy radical, as defined above, attached via an oxygen atom to a carbonyl radical. Examples of such alkoxycarbonyl radicals include methoxycarbonyl and t-butoxycarbonyl.

The term “alkoxycarbonylalkyl” embraces radicals having alkoxycarbonyl moiety, as defined above substituted to an alkyl radical. Examples of such alkoxycarbonylalkyl radicals include methoxycarbonylethyl (—(CH₂)₂(O═)COCH₃) and t-butoxycarbonylethyl (—(CH₂)₂(O═)COC(CH₃)₃).

The term “alkylaminoalkyl” embraces aminoalkyl radicals wherein the nitrogen atom is substituted with an alkyl radical.

The term “alkylcarbonylalkyl” denotes an alkyl radical substituted with an “alkylcarbonyl” radical.

The term “alkenyl,” used alone or within other terms such as “haloalkenyl,” embraces unsaturated linear or branched radicals having two to about twenty carbon atoms and containing at least one carbon-carbon double bond. Examples of alkenyl radicals include ethenyl, propenyl butenyl, pentenyl, and the like.

The term “cycloalkenyl” embraces unsaturated radicals having three to ten carbon atoms and containing at least one carbon-carbon double bond, and includes monocyclic, bicyclic, and tricyclic radicals. Examples of cycloalkenyl radicals include cyclopropenyl, cyclobutenyl, cyclopentenyl, cyclohexenyl, cycloheptenyl, decahydronaphthenyl, hexahydroindenyl, hexahydropentalenyl, bicyclo[2.1.0]pentenyl, bicyclo[1.1.1]pentenyl, bicyclo[2.1.1]hexenyl, bicyclo[2.2.1]heptenyl, bicyclo[3.1.1]heptenyl, bicyclo[3.2.1]octenyl, bicyclo[2.2.2]octenyl, and bicyclo[4.2.2]decenyl.

The term “alkynyl,” used alone or within other terms such as “haloalkynyl,” embraces unsaturated linear or branched radicals having two to about twenty carbon atoms and containing at least one carbon-carbon triple bond. Examples of alkynyl radicals include ethynyl, propynyl butynyl, pentynyl, and the like.

The term “aryl”, alone or in combination, means a carbocyclic aromatic system containing one, two, or three rings wherein at least one of the rings is aromatic, and wherein such rings may be attached together in a pendant manner or may be fused. Examples of aryl radicals include phenyl, naphthyl, tetrahydronapthyl, indyl, and biphenyl. Aryl moieties, alone or in combination, may be optionally substituted by one or more substituents selected from the group consisting of amino, halo, cyano, hydroxyl, alkyl, alkoxy, and carboxyl.

The term “aralkyl” embraces aryl-substituted alkyl radicals such as benzyl, diphenylmethyl, triphenylmethyl, phenethyl, and diphenethyl.

The term “arylsulfonyl” embraces aryl radicals as defined above attached to a sulfonyl radical.

The term “acyl,” whether used alone or within a term such as “acylamino,” denotes a radical provided by the residue after removal of hydroxyl from an organic acid.

The term “acylamino” embraces an amino radical substituted with an acyl group. An examples of an “acylamino” radical is acetylamino (CH₃C(═O)NH—).

The term “heterocyclic” or “heterocycle” means a saturated or unsaturated mono- or multi-ring carbocyclic system wherein one or more carbon atoms in the system are replaced by nitrogen, sulfur, phosphorous, and/or oxygen. The term “heterocyclic” embraces “heteroaryl” groups, which means a carbocyclic aromatic system containing one, two, or three rings wherein at least one of the rings is aromatic, wherein such rings may be attached together in a pendant manner or may be fused, and wherein one or more carbon atoms in the system are replaced by nitrogen, sulfur, phosphorous, and/or oxygen. “Heterocyclic” includes, for example, the following structures: 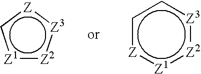
wherein Z, Z¹, Z², and Z³ are independently carbon, sulfur, phosphorous, oxygen, or nitrogen, with the proviso that one of Z, Z¹, Z², or Z³ is other than carbon, but is not oxygen or sulfur when attached to another Z atom by a double bond or when attached to another oxygen or sulfur atom. Furthermore, the optional substituents are understood to be attached to Z, Z¹, Z², or Z³ only when each is carbon. For example, the term “heterocyclyl” embraces each of the following groups, although this listing is not meant to limit the definition to these groups only: furanyl; thienyl; pyrrolyl; 2-isopyrrolyl; 3-isopyrrolyl; pyrazolyl; 2-isoimidazolyl; 1,2,3-triazolyl; 1,2,4-triazolyl; 1,2-dithiolyl; 1,3-dithiolyl; 1,2,3-oxathiolyl; isoxazolyl; oxazolyl; thiazolyl; isothiazolyl; 1,2,3-oxadiazolyl; 1,2,4-oxadiazolyl; 1,2,5-oxadiazolyl; 1,3,4-oxadiazolyl; 1,2,3,4-oxatriazolyl; 1,2,3,5-oxatriazolyl; 1,2,3-dioxazolyl; 1,2,4-dioxazolyl; 1,3,2-dioxazolyl; 1,3,4-dioxazolyl; 1,2,5-oxathiazolyl; 1,3-oxathiolyl; 1,2-pyranyl; 1,4-pyranyl; 1,2-pyranonyl; 1,4-pyranonyl; 1,2-dioxinyl; 1,3-dioxinyl; pyridyl; pyridazyl; pyrimidyl; pyrazinyl; piperazyl; 1,3,5-triazinyl; 1,2,4-triazinyl; 1,2,3-triazinyl; 1,2,4-oxazinyl; 1,3,2-oxazinyl; 1,3,6-oxazinyl; 1,2,6-oxazinyl; 1,4-oxazinyl; o-isoxazinyl; p-isoxazinyl; 1,2,5-oxathiazinyl; 1,4-oxazinyl; o-isoxazinyl; p-isoxazinyl; 1,2,5-oxathiainzyl; 1,2,6-oxathiainzyl; 1,4,2-oxadiainzyl; 1,3,5,2-oxadiainzyl; morpholino; azepinyl; oxepinyl; thiepinyl; 1,2,4-diazepinyl; benzofuranyl; isobenzofuranyl; benzothiofuranyl; isobenzothiofuranyl; indolyl; indoleninyl; 2-isobenzazolyl; 1,5-pyrindinyl; pyrano[3,4-b]pyrrolyl; isoindazolyl; indoxazinyl; benzoxazolyl; anthranilyl; 1,2-benzopyranyl; quinolyl; isoquinolyl; cinnolyl; quinazolyl; naphthyridyl; pyrido[3,4-b]pyridyl; pyrido[3,2-b]pyridyl; pyrido[4,3-b]pyridyl; 1,3,2-benzoxazyl; 1,4,2-benzoxazyl; 2,1,3-benzoxazyl; 3,1,4-benzoxazyl; 1,2-benzoisoxazyl; 1,4-benzoisoxazyl; carbazolyl; xanthenyl; acridinyl; purinyl; thiazolidyl; piperidyl; pyrrolidyl; 1,2-dihydroazinyl; 1,4-dihydroazinyl; 1,2,3,6-tetrahydro-1,3-diazinyl; perhydro-1,4-diazinyl; 1,2-thiapyranyl; and 1,4-thiapyranyl. Heterocyclic moieties, alone or in combination, may be optionally substituted by one or more substituents selected from the group consisting of amino, halo, cyano, hydroxyl, alkyl, alkoxy, and carboxyl.

The term “heteroaryl” also embraces radicals where heterocyclic radicals are fused with aryl radicals as defined herein. Examples of such fused bicyclic radicals include benzofuran, benzothiophene, and the like.

The term “heterocycloalkyl” embraces heterocyclic-substituted alkyl radicals such as pyridylmethyl and thienylmethyl.

The terms benzyl and phenylmethyl are interchangeable.

The phrases “combination therapy”, “co-administration”, “administration with”, or “co-therapy”, in defining the use of a selective IKK-2 inhibitory agent in combination with another therapeutic agent such as another analgesic agent, is intended to embrace administration of each agent in a sequential manner in a regimen that may provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

The term “subject” for purposes of treatment includes any human or animal subject who is in need of the prevention of, or who has pain, inflammation and/or any one of the known inflammation-associated disorders. The subject is typically a human subject.

The phrase “therapeutic combination” as used herein refers to the combination of two or more therapeutic compounds and, optionally, one or more pharmaceutically acceptable carrier used to provide dosage forms that produce a beneficial effect of each therapeutic compound in the subject at the desired time, whether the therapeutic compounds are administered substantially simultaneously, or sequentially.

The phrase “therapeutically effective” as used herein refers to an amount of a therapeutic compound, or amounts of combined therapeutic compounds in combination therapy. The amount or combined amounts achieve one or more of the goals of preventing, inhibiting, reducing or eliminating the inflammation or inflammation-related disease or condition. A “therapeutically-effective” amount of each agent in a combination therapy is expected to be less than an amount used in treatment using agent by itself, thus while avoiding adverse side effects typically associated with alternative therapies, namely higher dose monotherapy of each agent by itself.

The terms “treating” or “to treat” means to alleviate symptoms, eliminate the causation either on a temporary or permanent basis, or to prevent or slow the appearance of symptoms in a subject. The term “treatment” includes alleviation, elimination of causation of or prevention of pain and/or inflammation associated with, but not limited to, any of the diseases or disorders described above.

Pharmaceutically acceptable salts of the compounds of Formula I include the acid addition and base salts thereof.

Suitable acid addition salts are formed from acids that form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

Pharmaceutically acceptable salts of compounds of Formula I may be prepared by one or more of three methods: (i) by reacting the compound of Formula I with the desired acid or base; (ii) by removing an acid- or base-labile protecting group from a suitable precursor of the compound of Formula I or by ring-opening a suitable cyclic precursor, for example, a lactone or lactam, using the desired acid or base; or (iii) by converting one salt of the compound of Formula I to another by reaction with an appropriate acid or base or by means of a suitable ion exchange column. All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of the invention may exist in both unsolvated and solvated forms. The term “solvate” is used herein to describe a molecular complex comprising the compound of the invention and a stoichiometric amount of one or more pharmaceutically acceptable solvent molecules, for example, ethanol. The term “hydrate” is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionized, partially ionized, or non-ionized. For a review of such complexes, see Haleblian, J. Pharm. Sci., 64(8), 1269-1288 (1975).

Hereinafter all references to compounds of Formula I include references to salts, solvates and complexes thereof and to solvates and complexes of salts thereof.

The compounds of the invention include compounds of Formula I as hereinbefore defined, including all polymorphs and crystal habits thereof, prodrugs and isomers thereof (including optical, geometric and tautomeric isomers) as hereinafter defined and isotopically-labeled compounds of Formula I.

As indicated, so-called prodrugs of the compounds of Formula I are also within the scope of the invention. The term “prodrug” refers to a compound that is a drug precursor which, following administration to a subject and subsequent absorption, is converted to an active species in vivo via some process, such as a metabolic process. Other products from the conversion process are easily disposed of by the body. The more preferred prodrugs are those involving a conversion process that produces products that are generally accepted as safe.

Prodrugs in accordance with the invention can, for example, be produced by replacing appropriate functionalities present in the compounds of Formula I with certain moieties known to those skilled in the art as “pro-moieties.”

Some examples of prodrugs in accordance with the invention include: (i) where the compound of Formula I contains a carboxylic acid functionality (—CO₂H), an ester thereof, for example, a compound wherein the hydrogen of the carboxylic acid functionality of the compound of Formula I is replaced by C₁-C₈ alkyl;
(ii) where the compound of Formula I contains an alcohol functionality (—OH), an ether thereof, for example, a compound wherein the hydrogen of the alcohol functionality of the compound of Formula I is replaced by C₁-C₆ alkanoyloxymethyl; and (iii) where the compound of Formula I contains a primary or secondary amino functionality (—NH₂ or —NHR where R≠H), an amide thereof, for example, a compound wherein, as the case may be, one or both hydrogens of the amino functionality of the compound of Formula I is/are replaced by C₁-C₁₀ alkanoyl.

Further examples of replacement groups in accordance with the foregoing examples and examples of other prodrug types may be found in the aforementioned references.

Moreover, certain compounds of Formula I may themselves act as prodrugs of other compounds of Formula I.

Also included within the scope of the invention are metabolites of compounds of Formula I, that is, compounds formed in vivo upon administration of the drug. Some examples of metabolites in accordance with the invention include: (i) where the compound of Formula I contains a methyl group, an hydroxymethyl derivative thereof (—CH₃ →—CH₂OH); (ii) where the compound of Formula I contains an alkoxy group, an hydroxy derivative thereof (—OR⁴—OH); (iii) where the compound of Formula I contains a tertiary amino group, a secondary amino derivative thereof (—NR^{a}R^{b}→—NHR^{a} or —NHR^{b}); (iv) where the compound of Formula I contains a secondary amino group, a primary derivative thereof (—NHR→—NH₂); (v) where the compound of Formula I contains a phenyl moiety, a phenol derivative thereof (-Ph→-PhOH); and (vi) where the compound of Formula I contains an amide group, a carboxylic acid derivative thereof (—CONH₂—COOH).

Compounds of Formula I containing one or more asymmetric carbon atoms can exist as two or more stereoisomers. Where a compound of Formula I contains an alkenyl or alkenylene group, geometric cis/trans (or Z/E) isomers are possible. Where structural isomers are interconvertible via a low energy barrier, tautomeric isomerism (“tautomerism”) can occur. This can take the form of proton tautomerism in compounds of Formula I containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of Formula I, including compounds exhibiting more than one type of isomerism, and mixtures of one or more thereof. Also included are acid addition or base salts wherein the counterion is optically active, for example, d-lactate or I-lysine, or racemic, for example, dl-tartrate or dl-arginine.

Cis/trans isomers may be separated by conventional techniques well known to those skilled in the art, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (chiral HPLC).

Alternatively, the racemate (or a racemic precursor) may be reacted with a suitable optically active compound, for example, an alcohol, or, in the case where the compound of Formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2 to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art.

The present invention includes all pharmaceutically acceptable isotopically-labeled compounds of Formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁶F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labeled compounds of Formula I, for example, those incorporating a radioactive isotope, are useful in drug and/or substrate tissue distribution studies. The radioactive isotopes tritium (3H) and ¹⁴C are particularly useful for this purpose in view of their ease of incorporation and ready means of detection.

Substitution with heavier isotopes such as deuterium (2H) may afford certain therapeutic advantages resulting from greater metabolic stability, for example, increased in vivo half-life or reduced dosage requirements, and hence may be preferred in some circumstances.

Substitution with positron-emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula I can generally be prepared by conventional techniques known to those skilled in the art or by processes analogous to those described in the accompanying Examples using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, or d₆-DMSO.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

Generally, the compounds of the invention may be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

The compounds of the invention may be administered alone or in combination with one or more other compounds of the invention or in combination with one or more other drugs (or as any combination thereof). For example, compounds of Formula I may be used in co-therapies, partially or completely, in place of other conventional antiinflammatory therapies, such as together with other IKK-2 inhibitors, steroids, NSAIDs, COX-2 selective inhibitors, matrix metalloproteinase inhibitors, 5-lipoxygenase inhibitors, LTB₄ antagonists and LTA₄ hydrolase inhibitors.

Pharmaceutical compositions suitable for the delivery of compounds of the present invention and methods for their preparation will be readily apparent to those skilled in the art.

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Liang and Chen, Expert Opinion in Therapeutic Patents, 11(6), 981-986 (2001).

For tablet dosage forms, depending on dose, the drug may make up from 1 to 80 wt. % of the dosage form, more typically from 5 to 60 wt. % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 to 25 wt. %, preferably from 5 to 20 wt. % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 to 5 wt. % of the tablet, and glidants may comprise from 0.2 to 1 wt. % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 to 10 wt. %, preferably from 0.5 to 3 wt. % of the tablet.

Other possible ingredients include anti-oxidants, colorants, flavoring agents, preservatives and taste-masking agents.

Exemplary tablets contain up to about 80% drug, from about 10 to about 90 wt. % binder, from about 0 to about 85 wt. % diluent, from about 2 to about 10 wt. % disintegrant, and from about 0.25 to about 10 wt. % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

Consumable oral films for human or veterinary use are typically pliable water-soluble or water-swellable thin film dosage forms which may be rapidly dissolving or mucoadhesive and typically comprise a compound of Formula I, a film-forming polymer, a binder, a solvent, a humectant, a plasticiser, a stabilizer or emulsifier, a viscosity-modifying agent and a solvent. Some components of the formulation may perform more than one function.

The compound of Formula I may be water-soluble or insoluble. A water-soluble compound typically comprises from 1 to 80 wt. %, more typically from 20 to 50 wt. %, of the solutes. Less soluble compounds may comprise a greater proportion of the composition, typically up to 88 wt. % of the solutes. Alternatively, the compound of Formula I may be in the form of multiparticulate beads.

The film-forming polymer may be selected from natural polysaccharides, proteins, or synthetic hydrocolloids and is typically present in the range 0.01 to 99 wt. %, more typically in the range 30 to 80 wt. %.

Other possible ingredients include anti-oxidants, colorants, flavorings and flavor enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted- and programmed-release.

Suitable modified release formulations for the purposes of the invention are described in U.S. Pat. No. 6,106,864. Details of other suitable release technologies such as high energy dispersions and osmotic and coated particles are to be found in Verma et al., Pharmaceutical Technology On-line, 25(2), 1-14 (2001). The use of chewing gum to achieve controlled release is described in PCT Publication No. WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of Formula I used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted- and programmed-release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, e.g., Finnin and Morgan, J Pharm Sci, 88(10), 955-958 (1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted- and programmed-release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electrohydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 μm). This may be achieved by any appropriate comminuting method, such as spiral jet milling, fluid bed jet milling, supercritical fluid processing to form nanoparticles, high pressure homogenization, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of the compound of the invention per actuation and the actuation volume may vary from 1 to 100 μL. A typical formulation may comprise a compound of Formula I, propylene glycol, sterile water, ethanol and sodium chloride. Alternative solvents which may be used instead of propylene glycol include glycerol and polyethylene glycol.

Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted- and programmed-release.

In the case of dry powder inhalers and aerosols, the dosage unit is determined by means of a valve which delivers a metered amount. Units in accordance with the invention are typically arranged to administer a metered dose or “puff” containing from 20 to 1000 μg of the compound of Formula I. The overall daily dose will typically be in the range 100 μg to 10 mg which may be administered in a single dose or, more usually, as divided doses throughout the day, for example 2, 3, 4 or 8 times, giving for example, 1, 2 or 3 doses each time.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted- and programmed-release.

The compounds of the invention may also be administered directly to the eye or ear, typically in the form of drops of a micronized suspension or solution in isotonic, pH-adjusted, sterile saline. Other formulations suitable for ocular and aural administration include ointments, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted- or programmed-release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e., as a carrier, diluent, or solubilizer. Most commonly used for these purposes are α-, β- and γ-cyclodextrins, such as those described in PCT Publication No. WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Such kits comprises two or more separate pharmaceutical compositions, at least one of which contains a compound of Formula I in accordance with the invention, and means for separately retaining said compositions, such as a container, divided bottle, or divided foil packet. An example of such a kit is the familiar blister pack used for the packaging of tablets, capsules and the like.

Such kits are particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The amount of therapeutically active compounds that are administered and the dosage regimen for treating a disease condition with the compounds and/or compositions of this invention depends on a variety of factors, including the age, weight, sex and medical condition of the subject, the severity of the inflammation or inflammation related disorder, the route and frequency of administration, and the particular compound employed, and thus may vary widely. The pharmaceutical compositions may contain active ingredients in the range of about 0.1 to 1000 mg, preferably in the range of about 7.0 to 350 mg. A daily dose of about 0.01 to 100 mg/kg body weight, preferably between about 0.1 and about 50 mg/kg body weight and most preferably between about 0.5 to 30 mg/kg body weight, may be appropriate. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

These dosages are based on an average human subject having a weight of about 60 to 70 kg. The physician will readily be able to determine doses for subjects whose weight falls outside this range, such as infants and the elderly.

For the avoidance of doubt, references herein to “treatment” include references to curative, palliative and prophylactic treatment.

“DMF” is N,N-dimethylformamide.

“DMSO” is dimethylsulfoxide.

“ESI” is electrospray ionization Mass spectrometry.

“HATU” is O-(7-azabenzotriazol-1-yl)-N,N,N′,N′-tetramethyluronium hexafluorophosphate.

“HBTU” is O-benzotriazolo-1-yl)-N,N,N′,N′-tetramethyluronium.

“HRMS” is high resolution mass spectrometry.

“NMR” is nuclear magnetic resonance.

“Ph” is phenyl.

“i.d.” is inner diameter.

“R.O. water” is reverse osmosis water.

“EtOAc” is ethyl acetate.

“MCPBA” is meta-chloroperbenzoic acid.

“rt” is room temperature.

“h” is hour or hours.

“min” is minute or minutes.

### EXAMPLES

### Example

### 4-[(aminocarbonyl)amino]-1-[4-bromo-3-(trifluoromethyl)phenyl]-1H-pyrazole-3-carboxamide

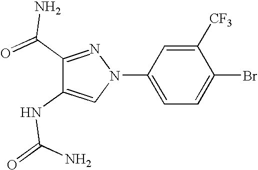

### Step 1: Preparation of (2E)-2-{[4-bromo-3-(trifluoromethyl)phenyl]hydrazono}-3-cyanopropanamide

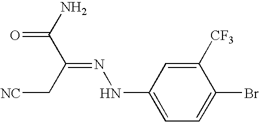

4-Bromo-3(trifluoromethyl)aniline (18.00 g, 75 mmol) was stirred with conc. HCl (18.75 mL, 187.5 mmol). After about 2-3 min. water (60 mL) was added and the reaction cooled to about −5° C. in an ice/acetone bath. A solution of sodium nitrite (5.174 g, 75 mmol) in water (18 mL) was added over about 15-20 min., maintaining the temperature between about −5 and 0° C. Meanwhile, a solution of cyano acetamide (18.9 g, 225 mmol) was prepared in ethanol (75 mL) and water (100 mL). A solution of sodium acetate trihydrate (10.5 g, 75 mmol) in 25 mL water was added and the mixture stirred in an ice/acetone bath. After the diazotization was complete a cold solution of sodium acetate trihydrate (30.6 g, 225 mmol) in water (75 mL) was added slowly, keeping the internal temperature of the reaction mixture about 0° C. The reaction mixture was then pipetted into the flask containing the cyano acetamide solution, 1 mL at a time over about 20 min. The temperature was maintained at 0 to −3° C. during the addition. After 1 h a milky brick red material was filtered and washed with water (2×150 mL), sucked dry for about 1 h then washed with ether (150 mL), removing much of the red color. After drying, an orangey solid was obtained and used directly in the next step.

### Step 2: Preparation of ethyl 4-amino-3-(aminocarbonyl)-1-[4-bromo-3-(trifluoromethyl)phenyl]-1H-pyrazole-5-carboxylate

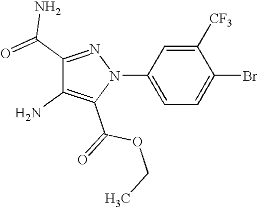

Crude hydrazone (13.94 g, 41.6 mmol) from the previous step and powdered anhydrous potassium carbonate (11.5 g, 83.2 mmol) were stirred in DMF (70 mL). Ethyl bromoacetate (11.1 g, 7.37 mL, 66.6 mmol) was added and the reaction heated to 130° C. for 1 h then allowed to cool to about 50° C.; triethylamine (8.42 g, 11.6 mL, 83.2 mmol) was then added. Upon cooling the reaction was poured into 650 mL ice/water mixture, filtered and washed with a little water. After drying, a dark blood-red solid was obtained which was used directly in the next step. ¹H NMR (300 MHz, DMSO-d₆): δ 1.31 (t, J=7.1 Hz, 3H), 4.17 (q, J=7.1 Hz, 2H), 5.80 (s, 2H), 7.41 (s, 1H), 7.74 (m, 2H), 8.03 (m, 2H).

### Step 3: Preparation of 4-amino-3-(aminocarbonyl)-1-[4-bromo-3-(trifluoromethyl)phenyl]-1H-pyrazole-5-carboxylic acid, potassium salt

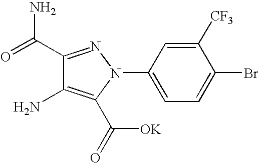

The crude ester (13.67 g, 32.5 mmol) from the previous step was stirred in 95% ethanol (100 mL) and a solution of 15% potassium hydroxide in ethanol (50 mL) was added. After 4 h the mixture was cooled to 0° C. and filtered. The precipitate was washed with ice cold ethanol (200 mL) and ether (200 mL) then dried to afford the potassium salt as a pinkish brown solid. This was used directly in the next step.

### Step 4: Preparation of 4-amino-1-[4-bromo-3-(trifluoromethyl)phenyl]-1H-pyrazole-3-carboxamide

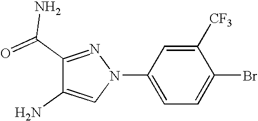

The potassium salt from the previous experiment (8.158 g, 18.9 mmol) was stirred with 85% phosphoric acid and heated to 75° C. for 2 h, and then cooled. Ice/water (150 mL) was added. Filtration gave a brown solid. ¹H NMR (300 MHz, DMSO-d₆): δ 7.32 (s, 1H), 7.73 (s, 1H), 7.96 (s, 2H), 8.00 (s, 1H), 8.04 (s, 1H), 8.31 (s, 1H); LCMS: m+=352, 350.

### Step 5: Preparation of 4-[(aminocarbonyl)amino]-1-[4-bromo-3-(trifluoromethyl)phenyl]-1H-pyrazole-3-carboxamide

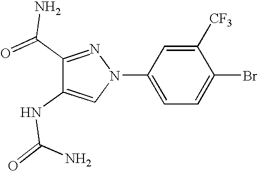

Crude amine (4.93 g, 14.12 mmol) from the previous step and potassium cyanate (3.436 g, 42.37 mmol) were stirred in THF (50 mL) and water (40 mL). Acetic acid (12 mL) was added and the mixture stirred for 1 hr. Ice/water was added and the resultant precipitate filtered and washed with water followed by ice cold ethanol (100 mL) and heptane (100 mL). The ethanol and heptane washings were evaporated to give an orange solid, which was chromatographed over silica gel (100 g). Elution with 0-10% CH₃OH—CHCl₃ gave the urea as a yellow solid. ¹H NMR (300 MHz, DMSO-d₆): δ 6.57 (s, 2H), 7.60 (s, 1H), 7.99 (d, J=9.0 Hz, 1H), 8.03 (s, 1H), 8.14 (dd, J=9.0, 2.4 Hz, 1H), 8.38 (d, J=2.4 Hz, 1H), 8.71 (s, 1H), 8.74 (s, 1H); LCMS: m−=392, 390.

### Example 2

### 4-({[(aminocarbonyl)amino]carbonyl}amino)-1-[4-bromo-3-(trifluoromethyl)phenyl]-1H-pyrazole-3-carboxamide

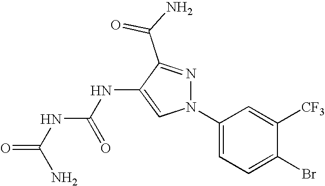

The filtered solid from Step 5 of Example 1 was identified as the desired product and isolated as a pinkish-gray solid. ¹H NMR (300 MHz, DMSO-d₆): δ 7.58 (s, 1H), 8.02 (m, 2H), 8.19 (m, 1H), 8.41 (m, 1H), 8.87 (m, 1H), 9.41 (s, 1H), 10.91 (s, 1H); LCMS: m+=437, 435; m−=435, 433.

### Example 3

### 4-[(aminocarbonyl)amino]-1-(4-bromo-3-ethoxyphenyl)-1H-pyrazole-3-carboxamide

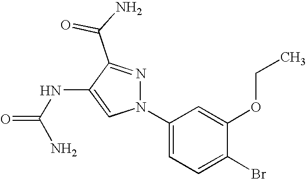

### Step 1: Preparation of (2E)-2-[(4-bromo-3-ethoxyphenyl)hydrazono]-2-cyanoacetamide

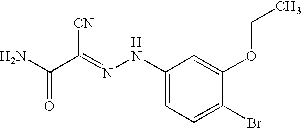

Concentrated HCl (11.9 mL, 119 mmol) was added over about 1-2 min. to 4-bromo-3-ethoxyaniline hydrochloride (20.00 g, 79.2 mmol) followed by water (65 mL) then cooled in an ice/acetone bath. A solution of sodium nitrite (5.46 g, 79.2 mmol) in water (20 mL) was added over about 15-20 min., maintaining the temperature between about −5 and 0° C. Meanwhile, a solution of cyano acetamide (20.0 g, 238 mmol) was prepared in ethanol (80 mL) and water (110 mL). A solution of sodium acetate trihydrate (11.0 g, 80.9 mmol) in water (25 mL) was added and the mixture stirred in an ice/acetone bath. After the diazotization was complete a cold solution of sodium acetate trihydrate (33.0 g, 242.6 mmol) in water (75 mL) was added slowly, keeping the internal temperature of the reaction mixture at about 0° C. This diazotization mixture was then pipetted into the cyano acetamide flask, 1 mL at a time over about 20 min. The reaction was filtered and the precipitate washed well with water, then dried. The hydrazone was obtained as a yellow solid. LCMS: m+=313, 311: m−=311, 309. This material was used directly in the next step.

### Step 2: Preparation of ethyl 4-amino-3-(aminocarbonyl)-1-(4-bromo-3-ethoxyphenyl)-1H-pyrazole-5-carboxylate

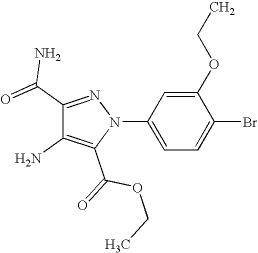

The hydrazone from the previous step (25.7 g, 82.6 mmol), anhydrous potassium carbonate (22.8 g, 165 mmol) and ethyl bromoacetate (22.1 g, 14.60 mL, 132 mmol) were heated to 130° C. in DMF (150 mL) for 4 h. The reaction mixture was cooled to 90° C., then triethylamine (16.7 g, 23.0 mL, 165 mmol) was added. The reaction mixture was cooled in ice and poured into ice/water (1400 mL). The precipitate was filtered and dried to afford the crude pyrazole as a brown solid. A small sample was chromatographed over silica gel (0-2% CH₃OH/CHCl₃) to give the title compound as a beige solid. ¹H NMR (300 MHz, DMSO-d₆): δ 1.11 (m, 3H), 1.31 (m, 3H), 4.12 (m, 4H), 5.70 (m, 2H), 6.96 (m, 1H), 7.22 (s, 1H), 7.33 (s, 1H), 7.62 (m, 2H); LCMS: m+=400, 398.

### Step 3: Preparation of 4-amino-3-(aminocarbonyl)-1-(4-bromo-3-ethoxyphenyl)-1H-pyrazole-5-carboxylic acid, potassium salt

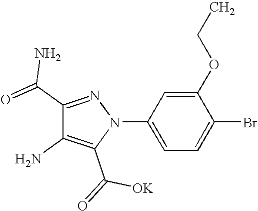

The crude ester from the previous step (19.156 g, 48.2 mmol) was stirred in 95% ethanol (180 mL) and a solution (90 mL) of 15% potassium hydroxide in ethanol was added; the reaction mixture was then heated at 45° C. for 4 h. The reaction mixutre was cooled in ice, filtered and the precipitate washed with ethanol (250 mL) and hexane (250 mL). The potassium salt was isolated as a khaki colored solid. LCMS: m+=372, 370; m−=369, 367. This was used directly in the next step.

### Step 4: Preparation of 4-amino-1-(4-bromo-3-ethoxyphenyl)-1H-pyrazole-3-carboxamide

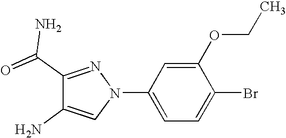

The potassium salt from the previous step (15.19 g, 37.3 mmol) was stirred with 85% phosphoric acid (50 mL) and heated at 75° C. for 2 h. After cooling, ice/water (600 mL) was added and the precipitate filtered. The product was obtained as a tan solid. A small sample was chromatographed over silica gel (0-3% CH₃OH—CHCl₃) to obtain the title compound as a beige solid. LCMS: m+=328, 326 and m−=325, 323; ¹H NMR (300 MHz, DMSO-d₆): δ 1.37 (t, J=6.9 Hz, 3H), 4.20 (q, J=6.9 Hz, 2H), 4.84 (s, 2H), 7.22 (s, 1H), 7.32 (m, 1H), 7.59 (m, 3H), 7.83 (s, 1H);

### Step 5: Preparation of 4-[(aminocarbonyl)amino]-1-(4-bromo-3-ethoxyphenyl)-1H-pyrazole-3-carboxamide

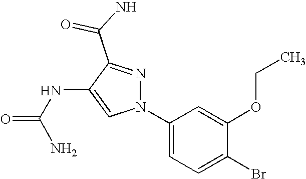

Potassium cyanate (3.38 g, 41.7 mmol) was added to a stirred suspension of amine (12.32 g, 37.9 mmol) from the previous step in a mixture of 2-propanol (180 mL), water (180 mL) and 75% acetic acid (180 mL). After 16 h water (450 mL) was added and the precipitate filtered. After washing with water (50 mL), CH₃OH (50 mL) and ether (100 mL), the urea was obtained as a tan solid. A small sample was chromatographed over silica gel (0-5% CH₃OH/CHCl₃) to afford the urea as a yellow solid. LCMS: m+=370, 368; m−=368, 366. ¹H NMR (300 MHz, DMSO-d₆): δ 1.37 (t, J=6.9 Hz, 3H), 4.22 (q, J=6.9 Hz, 2H), 6.51 (s, 2H), 7.40 (m, 1H), 7.61 (m, 3H), 7.88 (s, 1H), 8.60 (d, J=2.4 Hz, 1H), 8.65 (s, 1H).

### Example 4

### 4-[(aminocarbonyl)amino]-1-(4-iodo-2-methylphenyl)-1H-pyrazole-3-carboxamide

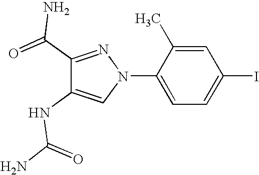

### Step 1: Preparation of (2E)-2-cyano-2-[(4-iodo-2-methylphenyl)hydrazono]acetamide

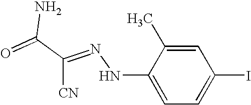

Preparation of Solution A: 2-Methyl-4-iodoaniline (23.3 g, 100 mmol) was treated with conc. HCl (25 mL) and diluted with water (100 mL). This mixture was stirred and cooled to −5 to 0° C. in an ice/acetone bath. To this was added a solution of sodium nitrite (6.90 g, 100 mmol) in water (20 mL) at −5 to 0° C. The resultant mixture was stirred 20 min. To this was added a −5° C. solution of sodium acetate trihydrate (40.8 g, 300 mmol) in water (100 mL).

Preparation of Solution B: Separately, cyanoacetamide (10.1 g, 120 mmol) was dissolved in water (180 mL) and ethanol (120 mL). The solution was cooled to −5° C. To this was added a cooled (−5° C.) solution of sodium acetate trihydrate (13.6 g, 100 mmol) in water (30 mL).

Solution A was added by pipette dropwise over about 30 min to Solution B at 5° C. to make a pasty mixture. After 3 h the mixture was filtered. The solids were then washed with ether (500 mL) and dried further to give a yellow brown solid. NMR indicated a mixture of stereomers.

### Step 2: Preparation of ethyl 4-amino-3-(aminocarbonyl)-1-(4-iodo-2-methylphenyl)-1H-pyrazole-5-carboxylate

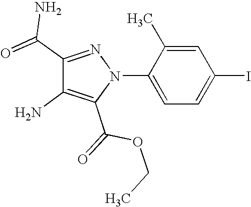

A mixture of the crude hydrazone from the previous step (20.0 g, 61.0 mmol), anhydrous potassium carbonate (16.8 g, 91.4 mmol) and ethyl bromoacetate (10.2 mL, 15.3 g, 122 mmol) in dry DMF was heated to 110° C. for 2 h. An additional 3.4 mL of ethyl bromoacetate (3.4 mL, 5.13 g, 30.7 mmol) was added. The reaction was allowed to cool and stand overnight then decanted from the solids and diluted with water (500 mL). The resultant solid precipitate was collected by filtration and dried under vacuum leaving a brown solid. A second precipitate was also collected and dried leaving another brown solid which was crystallized from ethyl acetate (75 mL) to give light tan crystals. LCMS: m+=416; ¹H NMR (300 MHz, DMSO-d₆): δ 1.05 (t, J=7.2 Hz, 3H), 1.95 (s, 3H), 4.09 (q, J=7.2 Hz, 2H), 5.69 (s, 2H), 7.08 (d, J=8.4 Hz, 1H), 7.32 (s, 1H), 7.65 (m, 2H), 7.78 (s, 1H).

### Step 3: Preparation of 4-amino-3-(aminocarbonyl)-1-(4-iodo-2-methylphenyl)-1H-pyrazole-5-carboxylic acid

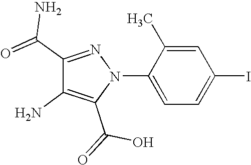

The ester from the previous step (20.7 g, 50 mmol) was treated with a 10% ethanolic solution of potassium hydroxide, stirred and warmed to 40° C. for 2 h. The reaction was cooled, treated with dry ice to about pH 9, diluted with water and extracted with ethyl acetate. The aqueous layer was neutralized to about pH 5 and the precipitate collected and dried giving the acid as a tan solid. LCMS: m+=388, m−=385; ¹H NMR (300 MHz, DMSO-d₆): δ 1.95 (s, 3H), 7.07 (d, J=8.0 Hz, 1H), 7.28 (s, 1H), 7.56 (m, 1H), 7.64 (d, J=8.0 Hz, 1H), 7.75 (s, 1H).

### Step 4: Preparation of 4-amino-1-(4-iodo-2-methylphenyl)-1H-pyrazole-3-carboxamide

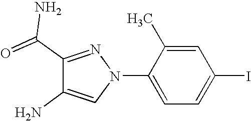

The carboxylic acid from the previous step (4.0 g, 10.4 mmol) was treated with 10.5 mL of 85% phosphoric acid (10.5 mL) and the mixture heated to 85° C. for 3 h. The reaction was then neutralized with a solution of sodium hydroxide (9.75 g) dissolved in water (50 mL). A sticky precipitate was formed which was dissolved in a mixture of THF and ethyl acetate then dried over magnesium sulfate. Filtration and evaporation provided a dark brown foamy gum which was dissolved in CH₃OH and treated with decolorizing carbon. Filtration and evaporation gave a brown gum. A small sample was chromatographed over silica gel (0-3% CH₃OH/CHCl₃) to provide the product as a yellow foam. LCMS: m+=344; ¹H NMR (300 MHz, DMSO-d₆): δ 2.20 (s, 3H), 4.77 (s, 2H), 7.14 (m, 2H), 7.34 (m, 2H), 7.65 (d, J=8.1 Hz, 1H), 7.76 (s, 1H).

### Step 5: Preparation of 4-[(aminocarbonyl)amino]-1-(4-iodo-2-methylphenyl)-1H-pyrazole-3-carboxamide

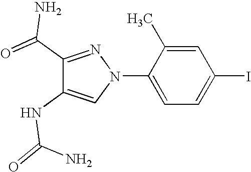

Crude amine from the previous step (264 mg, 0.77 mmol) was stirred in 2-propanol (4 mL), 75% acetic acid (4 mL) and water (4 mL). Potassium isocyanate (71 mg, 0.88 mmol) was added and the reaction stirred for 1 h. then the solvents evaporated to afford a damp sticky solid. Chromatography over silica gel (0-4% CH₃OH/CHCl₃) gave the urea as a pale yellow solid. LCMS: m+=386 and m−=384; ¹H NMR (300 MHz, DMSO-d₆): δ 2.19 (s, 3H), 6.46 (s, 2H), 7.19 (m, 1H), 7.42 (s, 1H), 7.65 (m, 2H), 7.79 (s, 1H), 8.12 (d, J=3.6 Hz, 1H), 8.67 (s, 1H).

### Example 5

### 4-[(aminocarbonyl)amino]-1-(3′-hydroxy-3-methyl-1,1′-biphenyl-4-yl)-1H-pyrazole-3-carboxamide

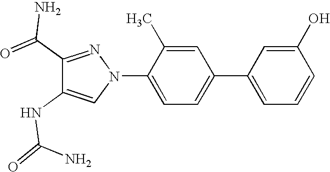

4-[(aminocarbonyl)amino]-1-(4-iodo-2-methylphenyl)-1H-pyrazole-3-carboxamide (Example 4, 117 mg, 0.30 mmol) was placed in a vial under N₂. 3-hydroxyphenylboronic acid (84 mg, 0.61 mmol) and tetrakis(triphenylphosphine)palladium(0) (53 mg, 0.046 mmol) were added followed by dry DMF (2 mL). N₂ was bubbled through the amber solution for 10 min. when a solution of Cs₂CO₃ (297 mg, 0.91 mmol) in water (0.5 mL) water was added. The reaction was heated to 80° C. for 16 h, cooled and added to ice/water (50 mL). Filtration gave a yellow solid which was chromatographed over silica gel (0-7% CH₃OH/CHCl₃) to provide the product as a cream solid. LCMS: m+=352 and m−=350. ¹H NMR (300 MHz, DMSO-d₆): δ 2.28 (s, 3H), 6.46 (s, 2H), 6.77 (m, 1H), 7.08 (m, 2H), 7.25 (m, 1H), 7.44 (m, 2H), 7.52 (m, 1H), 7.62 (m, 2H), 8.16 (s, 1H), 8.68 (s, 1H), 9.55 (s, 1H).

### Example 6

### 4-[(aminocarbonyl)amino]-1-(3-chloro-4-iodophenyl)-1H-pyrazole-3-carboxamide

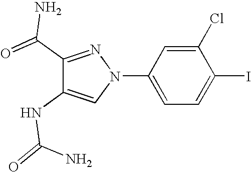

### Step 1: Preparation of (2E)-2-[(3-chloro-4-iodophenyl)hydrazono]-2-cyanoacetamide

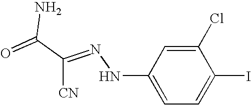

A 250 mL 2-neck flask, flushed with N₂, was charged with a stir bar and 3-chloro-4-Iodoaniline (19.01 g, 75 mmol) and was then cooled in acetone/ice bath to 0° C. To this was added conc. HCl (19 mL) dropwise at a rate that maintained the temperature below 5° C. Water (60 mL) was added followed by a solution of sodium nitrite (5.175 g, 75 mmol) in water (15 mL) over a 5 min period. After 75 mins, a solution of sodium acetate trihydrate (30.62 g, 225 mmol) in water (75 mL) was added over a 5 min. period with the temperature maintained at 5° C. The resultant yellowish slurry was then used as described in the following paragraph.

To a well-stirred solution of cyanoacetamide (12.61 g, 150 mmol) in water (115 mL) and ethanol (75 mL) in a 500-mL round bottom flask was added a solution of acetate trihydrate (10.5 g, 75 mmol) in water (25 mL) and this mixture was cooled to −7° C. in an acetone/water bath. To this solution was added the slurry from the previous paragraph dropwise over a 70-min period with the temperature maintained at 0° C. (±2° C.) during the addition. After 2.5 h, the cold mixture was filtered and the solids washed with water (3×100 mL) and air-dried. These solids were washed with CH₃OH (4×100 mL) and dried in vacuo to give a yellow solid. The CH₃OH washes were concentrated, cooled, and filtered to provide additional product. ¹H NMR (DMSO-d₆): δ 11.750 (m, 1H), 7.940 (m, 1H), 7.860 (m, 1H), 7.822 (m, 1H), 7.550 (m, 1H), 7.330 (m, 1H). MS (ESI) for C₉H₆ClIN₄O m/z 347.0 (M−H)⁻.

### Step 2: Preparation of ethyl 4-amino-3-(aminocarbonyl)-1-(3-chloro-4-iodophenyl)-1H-pyrazole-5-carboxylate

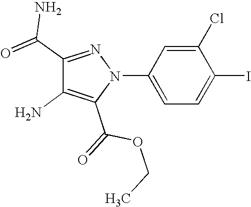

To a well stirred slurry of the hydrazone from the previous step (19 g, 55 mmol) and anhydrous potassium carbonate (15 g, 109 mmol) in DMF (150 mL) was added ethyl bromoacetate (6.1 mL, 55 mmol) and the reaction heated to 130° C. Additional aliquots of ethyl bromoacetate (3 mL, 17 mmol) were added at 3.5 h, 5.5 h and 6.25 h. After 6.5 h the reaction mixture was cooled, poured into ice/water (600 mL) and stored at 5° C. overnight. The slurry was then filtered, washed with water and dried in vacuo to give a reddish-brown solid. ¹H NMR (300 MHz, DMSO-d₆): δ 8.010 (m, 1H), 7.772 (m, 1H), 7.650 (m, 1H), 7.390 (m, 1H), 7.23 (m, 1H), 5.739 (m, 2H), 4.157 (m, 2H), 1.124 (m, 3H). MS (ESI+) for C₁₃H₁₂ClIN₄O₃ m/z 435.9 (M+H)⁺.

### Step 3: Preparation of 4-amino-3-(aminocarbonyl)-1-(3-chloro-4-iodophenyl)-1H-pyrazole-5-carboxylic acid, potassium salt

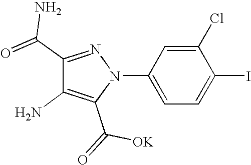

The ester from Step 2 (20.5 g, 47 mmol) was stirred with 15% KOH/ethanol (210 mL) at rt. After 2 h, the heterogeneous mix was transferred to freezer and stored overnight. The mixture was filtered, washed with cold ethanol (3×10 mL) and air-dried to give a reddish-brown solid.

### Step 4: Preparation of 4-amino-1-(4-iodo-2-methylphenyl)-1H-pyrazole-3-carboxamide

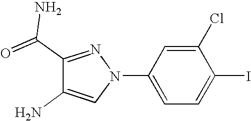

A slurry of phosphoric acid (50 mL, 85%) and the potassium salt from the preceeding step (9.5 g, 21.3 mmol) in a 500-mL round bottom flask was heated at 80° C. for 65 min. The reaction mixture was removed from heat, allowed to cool for 15 min, and then poured onto ice (500 mL). This mixture was stirred an additional 75 min, filtered and the solids washed with water (5×80 mL), aqueous sodium bicarbonate(1×30 mL) and air-dried to give a brown solid. ¹H NMR (300 MHz, DMSO-d₆): δ 8.158 (d, J=1.8 Hz, 1H), 8.011 (d, J=8.4 Hz, 1H), 7.872 (s, 1H), 7.670 (s, 1H), 7.594 (dd, J=2.7, 8.4 Hz, 1H), 7.298 (s, 1H), 4.90 (s, 1H). MS (ESI+) for C₁₀H₈ClIN₄O m/z 363.9 (M+H)⁺.

### Step 5: Preparation of 4-[(aminocarbonyl)amino]-1-(3-chloro-4-iodophenyl)-1H-pyrazole-3-carboxamide

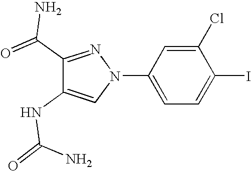

To a well stirred slurry of the amine from the previous step (2.62 g, 7 mmol) and potassium cyanate (1.32 g, 16 mmol) in CH₃OH (150 mL)/water (150 mL) at rt was added 70% acetic acid (80 mL). Additional aliquots of potassium cyanate were added at 60 min. (1.31 g, 16 mmol) and 18 h (0.330 g, 4 mmol). After 20 h, additional acetic acid solution was added (5 mL, 70%). At 24 h, the mixture was filtered, the solids washed with water (4×50 mL) and air-dried. This material was then washed with CH₃OH (3×50 mL) and CH₃OH/acetone (1/1, 6×100 mL). The latter CH₃OH/acetone extracts were combined, filtered and the filtrate concentrated to give a brown solid. ¹H NMR (300 MHz, DMSO-d₆): δ 8.684 (s, 1H), 8.646 (s, 1H), 8.220 (s, 1H), 8.00 (m, 2H), 7.62 (m, 2H), 6.548 (s, 2H). MS (ESI−) for C₁₁H₉ClIN₅O₂ m/z 404.0.

### Example 7

### 4-[(aminocarbonyl)amino]-1-(2-chloro-3′-hydroxy-1,1′-biphenyl-4-yl)-1H-pyrazole-3-carboxamide

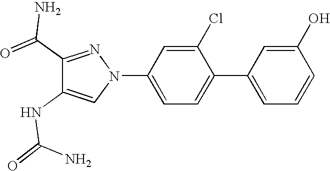

A 2-neck roundbottom flask was charged with 4-[(aminocarbonyl)amino]-1-(3-chloro-4-iodophenyl)-1H-pyrazole-3-carboxamide (Example 6, 0.214 g, 0.5 mmol), tetrakis(triphenylphosphine) Pd(0) (0.044 g, 0.038 mmol), 3-hydroxyphenylboronic acid (0.144 g, 1 mmol) and the mixture was flushed with N₂. An aqueous solution of Cs₂CO₃ (2M, 0.6 mL, 1.2 mmol, under N₂) was added via syringe followed by anhydrous DMF (5 mL, via syringe). The reaction was flushed again with N₂ and heated to 80° C. After 18 h, the reaction was removed from heat, poured onto ice (40 mL), and the mixture stirred for 10 min. The slurry was filtered and the collected solids washed with water (3×20 mL) and air dried to give a pale brown solid. ¹H NMR (300 MHz, DMSO-d₆): δ 9.561 (s, 1H), 8.649 (m, 2H), 8.139 (m, 1H), 7.885 (m, 2H), 7.470 (m, 2H), 7.245 (m, 1H), 6.815 (m, 3H), 6.508 (s, 2H). MS (ESI−) for C₁₇H₁₄ClN₅O₃ m/z 370.2 (M−H)⁻.

### Example 8

### 4-[(aminocarbonyl)amino]-1-(4-iodo-3-methylphenyl)-1H-pyrazole-3-carboxamide

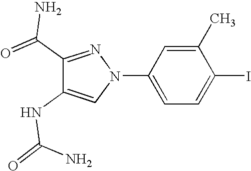

### Step 1: Preparation of (2E)-2-cyano-2-[(4-iodo-3-methylphenyl)hydrazono]acetamide

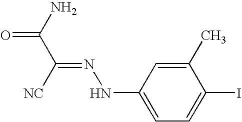

A mechanically stirred slurry of 3-methyl-4-iodoaniline (23.3 g, 0.1 mole) and concentrated HCl (25 mL) in water (80 mL) was cooled to 5° C. and a cold solution of sodium nitrite (6.9 g, 0.1 mole, dissolved in 20 mL water) was added slowly over a period of 30 min., maintaining the reaction temperature at 0-5° C. A solution of sodium acetate trihydrate (40.8 g, 0.3 mole) in water (100 mL) was then added dropwise over 15-30 min.

This mixture was then slowly added to a cold (5° C.) mechanically stirred solution of sodium cyanoacetamide [previously prepared by the dissolution of cyanoacetamide (12.6 g, 0.15 mol) in cold (5° C.) water (150 mL)/ethanol (100 mL) followed by the addition of a cold solution of sodium acetate trihydrate (13.6 g, 0.1 mol) in water (30 mL)] over a period of 30 min. A thick brown slurry was obtained which was stirred for 3 h in a wet ice bath. The slurry was filtered and the solids washed with water (200 mL). The material was dried in vacuo to give a reddish brown solid. ¹H NMR (300 MHz, DMSO-d₆): δ 8.13 (s, 1H), 8.07-7.98 (m, 3H), 7.81 (s, 1H), 7.50 (d, J=8.3 Hz, 1H), 2.65 (s, 3H). MS (ESI−) for C₁₀H₉IN₄O m/z 327 (M-H)—.

### Step 2: Preparation of ethyl 4-amino-3-(aminocarbonyl)-1-(4-iodo-3-methylphenyl)-1H-pyrazole-5-carboxylate

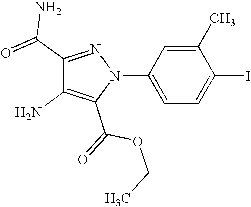

A slurry of freshly ground potassium carbonate (9.75 g, 70.6 mmol), ethyl bromoacetate (7.86g, 47 mmol), and the hydrazone from the previous step (10.29 g, 31.4 mmol) was heated to 90° C. under N₂ for 2.5 h. The solution was cooled and poured into ice (500 g). The aqueous mixture was then stirred at rt for 3 h and the precipitate was collected, washed with water and dried in vacuo to give a reddish-brown solid. ¹H NMR (300 MHz, DMSO-d₆): δ 7.87 (d, J=8.2 Hz, 1H), 7.58 (s, 1H), 7.44 (s, 1H), 7.33 (s, 1H), 7.02 (d, J=8.2 Hz, 1H), 5.7 (s, 2H), 4.13 (q, J=7.03 Hz, 2H), 2.38 (s, 3H), 1.10 (t, J=7.03 Hz, 3H). MS (ESI−) for C₁₄H₁₅IN₄O₃ m/z 413 (M−H).

### Step 3: Preparation of 4-amino-3-(aminocarbonyl)-1-(4-iodo-3-methylphenyl)-1H-pyrazole-5-carboxylic acid, potassium salt

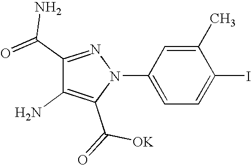

A slurry of potassium hydroxide (8.20 g, 148 mmol) and the ester from the previous step (8.23 g, 19.9 mmol) in ethanol (80 mL) was stirred at rt for 48 h. The solution was cooled to 5° C., filtered and the resulting solid dried in vacuo to a brown solid. ¹H NMR (300 MHz, DMSO-d₆): δ 7.72 (dd, J=8.6, 4.2 Hz, 1H), 7.36 (m, 1H), 7.18 (s, 1H), 7.00-6.96 (m, 2H), 5.27 (s, 2H), 2.33 (s, 3H). MS (ESI−) for C₁₂H₁₀₁KN₄O₃ m/z 385 (M⁻).

### Step 4: Preparation of 4-amino-1-(4-iodo-3-methylphenyl)-1H-pyrazole-3-carboxamide

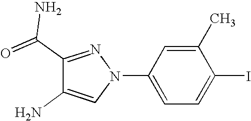

A well stirred slurry of the potassiuim salt from the previous step (2.40 g, 5.7 mmol) and phosphoric acid (10 mL) was heated to 45° C. for 2 h. The temperature was gradually increased at 60 min. intervals until gas evolution ceased. The final temperature was 65° C. The reaction mixture was cooled in an ice bath, neutralized with 50% sodium hydroxide and the mixture was repeatedly extracted with CHCl₃. The organics were combined, dried over anhydrous sodium sulfate and concentrated to give a red solid. ¹H NMR (300 MHz, DMSO-d₆): δ 7.86-7.83 (m, 2H), 7.76 (s, 1H), 7.49 (s, 1H), 7.41 (dd, J=8.3, 2.3 Hz, 1H), 7.22 (s, 1H), 4.85 (s, 2H), 2.39 (s, 3H). MS (ESI+) for C₁₁H₁₁N₄O m/z 343 (M+H)⁺.

### Step 5: Preparation of 4-[(aminocarbonyl)amino]-1-(4-iodo-3-methylphenyl)-1H-pyrazole-3-carboxamide

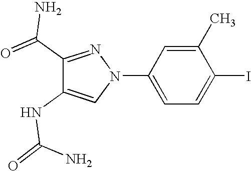

To a slurry of the amine prepared in the previous step (650 mg, 1.90 mmol) and potassium cyanate (185 mg, 2.28 mmol) in a solvent mixture of isopropanol/water (1:1, 20 mL) was added 75% acetic acid (10 mL) and the reaction mixture stirred at rt for 5 h. The mixture was diluted with water (30 mL) and the precipitate filtered, washed with water and air dried to give an off-white solid. This solid was dissolved in 1 mL of anhydrous DMSO and then crystallized through the careful addition of dichloromethane. This solid was filtered to provide the title compound. NMR (300 MHz, DMSO-d₆): δ 8.65 (s, 1H), 8.56 (m, 1H), 7.89-7.89 (m, 2H), 7.78 (s, 1H), 7.50-7.47 (m, 2H), 6.49 (s, 2H), 2.42 (s, 3H). MS (ESI−) for C₁₂H₁₂IN₅O₂,H m/z 384 (M−H)⁻.

### Example 9

### 4-[(aminocarbonyl)amino]-1-[4-(morpholin-4-yl-carbonyl)phenyl]-1H-pyrazole-3-carboxamide

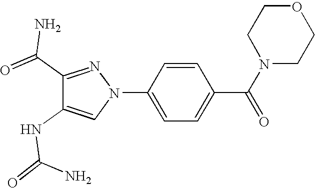

A suspension of 4-[(aminocarbonyl)amino]-1H-pyrazole-3-carboxamide (150 mg, 0.89 mmol) (prepared according to Example 19, from 4-amino-1H-pyrazole-3-carboxamide, see J. Am. Chem. Soc. 78, 2418-2422 (1956)) in pyridine (2.0 mL) and CH₂Cl₂ (1.0 mL) was treated with 4-(morpholine-4-carbonyl)phenylboronic acid (208 mg, 0.89 mmol) and Cu(OAc)₂ (161 mg, 0.89 mmol). The reaction was stirred under N₂ overnight. The crude mixture was concentrated, and redissolved in DMF (2 mL). The reaction mixture was then filtered through a syringe filter (0.45 μm), purified by prep. rpHPLC, and lyophilized to give the title compound as a white solid. ESI mass spectrum for C₁₆H₁₉N₆O₄⁺: 359.18 (M+1).

### Example 10

### 4-[(aminocarbonyl)amino]-1-(4-methoxyphenyl)-1H-pyrazole-3-carboxamide trifluoroacetate

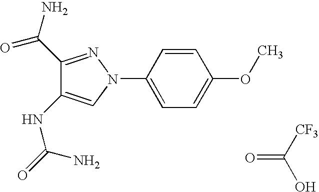

Prepared according to the same procedure as Example 9. ¹H NMR (300 MHz, DMSO-d₆): δ 3.80 (s, 3H) 6.52 (s, 2H) 7.02 (d, 2H) 7.49 (m, 1H) 7.70 (s, 1H) 7.77 (d, 2H) 8.49 (s, 1H) 8.67 (s, 1H). Mass of molecular ion (M+H): 276.1.

### Example 11

### 4-[(aminocarbonyl)amino]-1-[3-(3,4-dimethoxybenzyl)phenyl]-1H-pyrazole-3-carboxamide

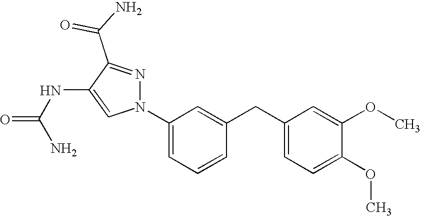

To a solution of 3,4-dimethoxybenzylzinc chloride (0.5 M in THF, 3.1 mL, 3.1 mmol) was added 4-[(aminocarbonyl)amino]-1-(3-bromophenyl)-1H-pyrazole-3-carboxamide (Example 20, 100 mg, 0.31 mmol), Pd(OAc)₂ (6.90 mg, 0.031 mmol) and triphenylphosphine (16.14 mg, 0.062 mmol). The reaction mixture was reflushed with Ar three times. The reaction was heated to 40° C. for 16 h in a sealed tube. The reaction was cooled to rt, filtered through a syringe filter (0.45 μm), purified by prep. rpHPLC, and lyophilized to give the title compound as a white solid. ¹H NMR (400 MHz, DMSO-d₆): δ 3.68 (s, 3H) 3.70 (s, 3H) 3.92 (s, 2H) 6.49 (m, 2H) 6.75 (dd, J=8.19, 2.01 Hz, 1H) 6.84 (d, J=8.32 Hz, 1H) 6.89 (d, J=1.88 Hz, 1H) 7.17 (m, 1H) 7.38 (t, J=7.92 Hz, 1H) 7.48 (m, 1H) 7.67 (m, 1H) 7.73 (m, 1H) 7.77. (t, J=1.75 Hz, 1H) 8.53 (s, 1H) 8.65 (s, 1H). Mass of molecular ion (M+H): 396.1658.

### Example 12

### 4-[(aminocarbonothioyl)amino]-1-(3-bromophenyl)-1H-pyrazole-3-carboxamide

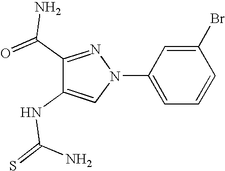

4-Amino-1-(3-bromophenyl)-1H-pyrazole-3-carboxamide (281 mg, 1 mmol, prepared according to the process of Example 214, Steps 1-3) and ammonium thiocyanate (228 mg, 3 mmol) was dissolved in a mixture of conc. HCl (0.15 mL) and distilled water (1 mL). The reaction was stirred at 80° C. overnight. After cooling to rt, a white solid precipitated out of the crude mixture. The solid was collected by filtration and washed several times with water and cold CH₃QH to give the title compound as a white solid. ¹H NMR (300 MHz, DMSO-d₆): δ 7.45-7.60 (m, 3H) 7.68 (s, 1H) 7.86 (m, 1H) 8.04 (s, 2H) 8.18 (t, J=1.91 Hz, 1H) 9.31 (m, 1H) 9.83 (s, 1H). Mass of molecular ion (M+H): 340.0.

### Example 13

### 4-[(aminocarbonyl)amino]-1-[3-(2-chloropyridin-4-yl)phenyl]-1H-pyrazole-3-carboxamide

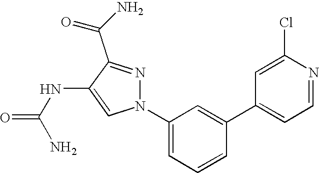

2-chloropyridine-4-boronic acid (37.4 mg, 0.2 mmol), 4-[(aminocarbonyl)amino]-1-(3-bromophenyl)-1H-pyrazole-3-carboxamide (Example 20, 78 mg, 0.24 mmol), and bis(triphenylphosphino)palladium dichloride (14 mg, 0.01 mmol) were sequentially added to degassed DMF (1 mL). The mixture was stirred at rt for 30 min. Degassed 2M aqueous Cs₂CO₃ (0.3 mL) was added to the mixture, and the reaction mixture was heated to 95° C. overnight. The reaction was cooled to rt, filtered through a syringe filter (0.45 μm), purified by prep. rpHPLC, and lyophilized to give the title compound as a white solid. ¹H NMR (300 MHz, DMSO-d₆): δ 6.51 (m, 2H) 7.55 (m, 1H) 7.65 (t, J=7.95 Hz, 1H) 7.81 (d, J=7.85 Hz, 1H) 7.90 (m, 2H) 8.03 (m, 2H) 8.35 (t, J=1.81 Hz, 1H) 8.51 (d, J=5.24 Hz, 1H) 8.68 (s, 1H) 8.76 (s, 1H). Mass of molecular ion (M+H): 357.1.

### Example 14

### 4-[(aminocarbonyl)amino]-1-(4-methoxyphenyl)-1H-pyrazole-3-carboxamide trifluoroacetate

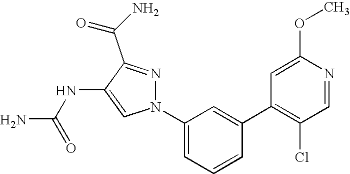

Prepared according to the same procedure as Example 13. ¹H NMR (300 MHz, DMSO-d₆): δ 3.31 (s, 3H) 6.50 (m, 2H) 7.32 (m, 1H) 7.50 (m, 4H) 7.76 (m, 1H) 7.87 (m, J=7.65 Hz, 2H) 8.55 (s, 1H) 8.67 (s, 1H). Mass of molecular ion (M+H): 387.6.

### Example 15

### 1-(3-bromophenyl)-4-{[(methylamino)carbonyl]amino}-1H-pyrazole-3-carboxamide

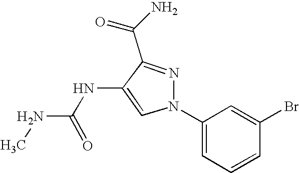

A solution of 4-amino-1-(3-bromophenyl)-1H-pyrazole-3-carboxamide (100 mg, 0.37 mmol, prepared according to the process of Example 214, Steps 1-3) was treated with methyl isocyanate (23 mg, 0.40 mmol) in THF (1 mL). The reaction was stirred under N₂ overnight. LC-MS indicated that the reaction was not complete, methyl isocyanate (23 mg, 0.40 mmol) was added to the mixture, and the reaction was stirred for another 4 h. Water (10 mL) was added to the mixture, and a white solid precipitated out of the solution. The solid was filtered, and washed several times with water and cold CH₃OH to give the title compound as a white solid. ¹H NMR (300 MHz, DMSO-d₆): δ 2.63 (d, J=4.63 Hz, 2H) 7.24 (m, 2H) 7.43 (m, 3H) 7.87 (m, 2H) 8.19 (m, 1H) 8.63 (s, 1H) 8.67 (s, 1H). Mass of molecular ion (M+H): 338.1.

Examples 16 and 17 were prepared according to the same procedure as Example 19.

| | | | MS (ES⁺) |
|---|---|---|---|
| Ex. | Name and Structure | ¹H NMR | (M + 1) |
| 16 | 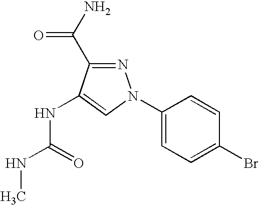 | (300 MHz, DMSO-d₆): δ2.63(d, J=4.63 Hz, 2 H), 7.20(m, 2H), 7.43 (s, 1H), 7.68(d, 2H), 7.80(m, 3H), 8.58(s, 1 H), 8.67(s, 1H) | 338.1 |
| 17 | 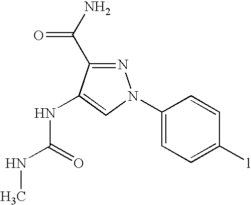 | (400 MHz, DMSO-d₆): δ2.61(d, J=3.76 Hz, 3 H), 7.23(s, 1H), 7.51(s, 1H), 7.76(m, 5H), 8.57 (s, 1H), 8.67(s, 1H) | 386.1 |

### Example 18

### 4-[(aminocarbonyl)amino]-1-(4-methylphenyl)-1H-pyrazole-3-carboxamide

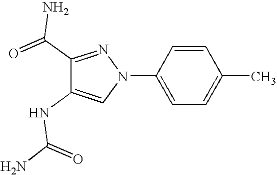

A suspension of 4-[(aminocarbonyl)amino]-1H-pyrazole-3-carboxamide (30 mg, 0.18 mmol) in DMF (1.0 mL) was treated with p-tolyboronic acid (29 mg, 0.22 mmol) and Cu(1)CI (3.4 mg, 0.03 mmol). Pyridine (0.022 mL) was added to the mixture. The vial was sealed with a crimped cap and the reaction mixture was heated in the CEM Microwave instrument at 75° C. for 2 h at full power (300 W). The reaction was cooled to rt, filtered through a syringe filter (0.45 μm), purified by prep. rpHPLC, and lyophilized to give the title compound as a white solid. ¹H NMR (300 MHz, DMSO-d₆): δ 2.33 (s, 3H) 6.48 (s, 2H) 7.29 (d, J=8.66 Hz, 2H) 7.49 (s, 1H) 7.73 (m, 3H) 8.49 (s, 1H) 8.65 (s, 1H). Mass of molecular ion (M+H): 260.1.

### Example 19

### 4-[(aminocarbonyl)amino]-1-(4-bromophenyl)-1H-pyrazole-3-carboxamide

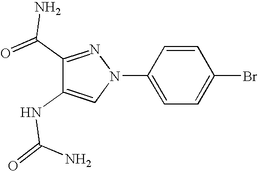

4-Amino-1-(4-bromophenyl)-1H-pyrazole-3-carboxamide (5 g, 17.8 mmol, prepared according to the process of Example 214, Steps 1-3) and sodium cyanate (4.045 g) were partially dissolved in a mixture of acetic acid (70 mL) and water (20 mL). The suspension was stirred overnight at rt. Most of solvents were removed under reduced pressure and water (100 mL) was added to the residue. After trituration for 3 h, the solid was filtered, washed with 2N sodium bicarbonate solution, and dried. The dry solid was dissolved in a minimal amount of DMSO. The obtained solution was added with stirring to 1 L of CH₂Cl₂ and after 1 h it was filtered and the solid dried to give the title compound. ¹H NMR (DMSO-d₆): δ 6.51 (br s, 2H), 7.54 (br s, 1H), 7.68 (dd, 2H, J=4.9, 2.0 Hz), 7.82 (br s, 1H), 7.86 (dd, 2H, J=5.0, 1.9 Hz), 8.58 (s, 1H), 8.67 (s, 1H). Mass of molecular ion (M+1): 324.

### Example 20

### 4-[(aminocarbonyl)amino]-1-(3-bromophenyl)-1H-pyrazole-3-carboxamide

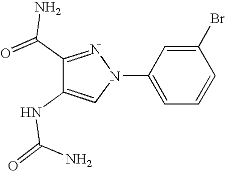

Prepared according to the same procedure as Example 19. ¹H NMR (300 MHz, DMSO-d₆): δ 6.52 (s, 2H) 7.43 (t, J=8.06 Hz, 1H) 7.49 (m, 1H) 7.52 (m, 1H) 7.86-7.92 (m, 2H) 8.19 (t, J=1.91 Hz, 1H) 8.61 (s, 1H) 8.67 (s, 1H). ESI mass spectrum for C₁₁H₁₁BrN₅O₂⁺: 324.0059 (M+1).

Examples 21-95: 4-[(aminocarbonyl)amino]-1-(4-arylphenyl)-1H-pyrazole-3-carboxamides

4-[(aminocarbonyl)amino]-1-(4-bromophenyl)-1H-pyrazole-3-carboxamide (Example 19, 0.19 g, 0.6 mmol), Pd[PPh₃]₄(50 mg), and 0.9 mmol of an arylboronic acid were added to a flask and placed under vacuum then refilled with N₂. The vacuum/N₂ cycle was repeated twice. DMF (3 mL) and 0.9 mL of 2M Cs₂CO₃ solution were injected under N₂. (The DMF and Cs₂CO₃ solutions were bubbled with N₂ for 15 min before addition). The suspension was heated and stirred at 80° C. overnight. Most of liquids were removed under reduced pressure. The solid was triturated with water (80 mL), filtered, triturated with CH₃OH (5-10 mL) for 1-3 h, filtered and dried to give the compounds shown in the following table.

| | | | MS (ES⁺) |
|---|---|---|---|
| Ex. | Name and Structure | ¹H NMR | (M + 1) |
| 21 | 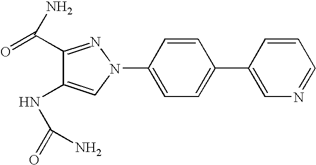 | (CD₃OD/DMSO-d₆(4:1)): δ 7.42-7.50(m, 1H), 7.82(d, 2H, J=8.7 Hz), 7.96(d, 2H, J=8.7 Hz), 8.04-8.14(m, 1H), 8.49-8.56(m, 1H), 8.59 (s, 1H), 8.89(s, 1H) | 323 |
| 22 | 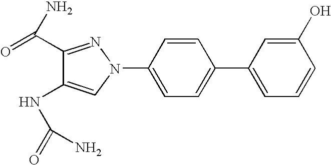 | (CD₃OD/DMSO-d₆(4:1)): δ 6.56(dm, 1H, J=6.4 Hz), 7.08(t, 1H, J=1.7 Hz), 7.12(d, 1H, J=7.8 Hz), 7.25(t, 1H, J=7.9 Hz), 7.73(d, 2H, J=8.8 Hz), 7.88(d, 2H, J=8.7 Hz), 8.55(s, 1H) | 338 |
| 23 | 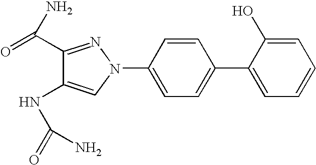 | (CD₃OD/DMSO-d₆(4:1)): δ 6.79-6.86(m, 2H), 7.04-7.12(m, 1H), 7.22(dd, 1H, J=6.2, 1.4 Hz), 7.62(d, 2H, J=8.7 Hz), 7.78(d, 2H, J=8.7 Hz), 8.47(s, 1H) | 338 |
| 24 | 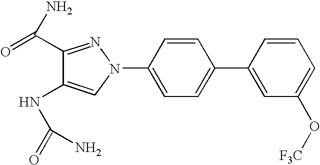 | (CD₃OD): δ 7.18(d, 1H, J=8.2 Hz), 7.42-7.62 (m, 2H), 7.68(d, 1H, J=8.1 Hz), 7.79(d, 2H, J=8.7 Hz), 7.95(d, 2H, J=8.7 Hz), 8.57(s, 1H) | 406 |
| 25 | 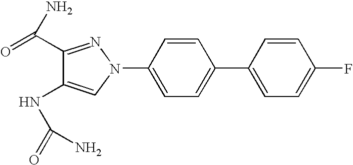 | (DMSO-d₆): δ 6.50(br s, 2H), 7.29(t, 2H, J=8.8 Hz), 7.53(br s, 1H), 7.70-7.87(m, 5H), 7.89(d, 2H, J=8.5 Hz), 8.62(s, 1H), 8.69(s, 1H) | 340 |
| 26 | 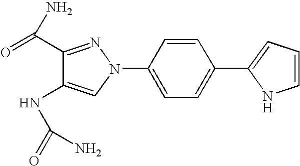 | (CD₃OD): δ 5.52-5.61 (m, 1H), 6.52-6.62(s, 1H), 6.71-7.77(m, 1H), 7.68(dd, 2H, J=8.7 Hz), 7.77(d, 2H, J=8.7 Hz), 8.49(s, 1H) | 311 |
| 27 | 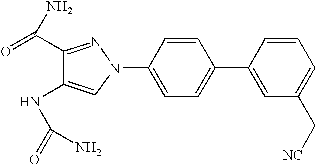 | (DMSO-d₆): δ 4.11(s, 2H), 6.55(br s, 2H), 7.37(d, 1H, J=7.8 Hz), 7.48-7.56(m, 2H), 7.66-7.73(m, 2H), 7.79 (d, 3H, J=8.7 Hz), 7.99 (d, 2H, J=8.7 Hz), 8.60 (s, 1H), 8.72(s, 1H) | 361 |
| 28 | 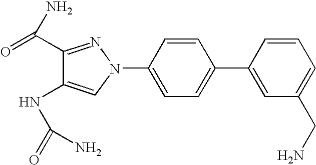 | (CD₃OD/DMSO-d₆(4:1)): δ 3.79(s, 2H), 7.26(d, 1H, J=7.5 Hz), 7.35(t, 1H, J=7.6 Hz), 7.79(d, 2H, J=8.7 Hz), 7.72(d, 1H, J=8.8 Hz), 7.62(s, 1H), 7.84(d, 2H, J=8.8 Hz), 8.50(s, 1H) | 351 |
| 29 | 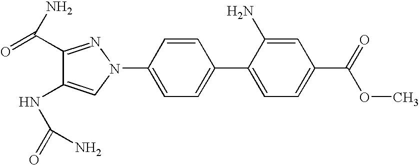 | (DMSO-d₆): δ 3.92(s, 3H), 5.18(s, 2H), 6.55 (br s, 2H), 7.15(d, 1H, J=7.9 Hz), 7.19(dd, 1H, J=6.3, 1.5 Hz), 7.42(d, 1H, J=1.5 Hz), 7.55(br s, 1H), 7.59(d, 2H, J=8.7 Hz), 7.90(br s, 1H)_, 7.99(d, 2H, J=8.7 Hz), 8.60(s, 1H), 8.70(s, 1H) | 395 |
| 30 | 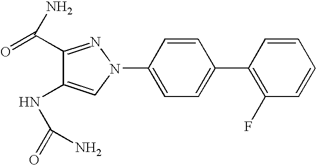 | (CD₃OD/DMSO-d₆(4:1)): δ 7.15-7.28(m, 2H), 7.30-2.40(m, 1H), 7.46-7.56(m, 1H), 7.63, (d, 2H, J=8.7 Hz), 7.88 (d, 2H, J=8.7 Hz), 8.55 (s, 1H) | 340 |
| 31 | 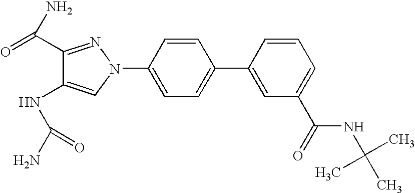 | (CD₃OD/DMSO-d₆(4:1)): δ 1.40(s, 9H), 7.46(t, 1H, J=7.8 Hz), 7.67(d, 1H, J=8.0 Hz), 7.73(s, 1H), 7.77(d, 2H, J=8.9 Hz), 7.94-8.00(m, 1H), 8.52(s, 1H) | 421 |
| 32 | 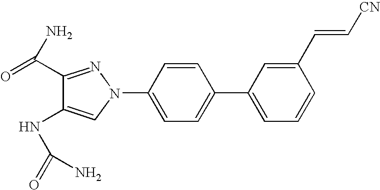 | (DMSO-d₆): δ 6.52(br s, 2H), 2.64(d, 1H, J=16.7 Hz), 7.45-7.60 (m, 2H), 7.66(d, 1H, J=7.8 Hz), 7.79-7.92 (m, 4H), 7.96-8.08(m, 4H), 8.63(s, 1H), 8.71 (s, 1H) | 373 |
| 33 | 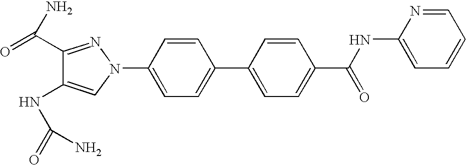 | (DMSO-d₆): δ 6.52(br s, 2H), 7.18(t, 1H, J=6.1 Hz), 7.55(br s, 1H), 7.80-7.90(m, 6H), 8.02(d, 2H, J=8.6 Hz), 8.15(d, 2H, J=8.5 Hz), 8.20(d, 1H, J=8.5 Hz), 8.38(d, 1H, J=3.4 Hz), 8.65(s, 1H), 8.70(s, 1H) | 442 |
| 34 | 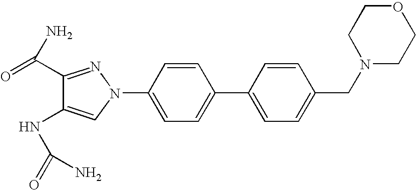 | (CD₃OD/DMSO-d₆(4:1)): δ 2.20-2.40(m, 4H), 3.50-3.70(m, 4H), 7.33(d, 2H, J=7.7 Hz), 7.56(d, 2H, J=7.7 Hz), 7.67(d, 2H, J=8.3 Hz), 7.81(d, 2H, J=8.2 Hz), 8.47(s, 1H) | 421 |
| 35 | 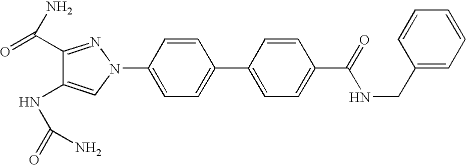 | (DMSO-d₆): δ 4.50(d, 2H, J=5.7 Hz), 6.51(br s, 2H), 7.20-7.40(m, 5H), 7.53(br s, 1H), 7.78-7.89(m, 5H), 7.98 (d, 4H, J=8.1 Hz), 8.62 (s, 1H), 8.68(s, 1H), 9.20(t, 1H, J=5.7 Hz) | 455 |
| 36 | 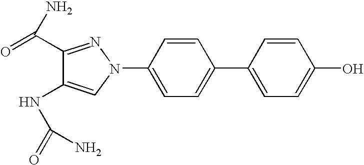 | (CD₃OD/DMSO-d₆(4:1)): δ 6.81(d, 2H, J=8.6 Hz), 7.46(d, 2H, J=8.6 Hz), 7.63(d, 2H, J=8.7 Hz), 7.79(d, 2H, J=8.7 Hz), 8.50(s, 1H) | 338 |
| 37 | 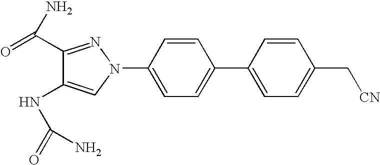 | (CD₃OD/DMSO-d₆(4:1)): δ 3.97(s, 2H), 7.40(d, 2H, J=8.2 Hz), 7.67(d, 2H, J=8.2 Hz), 7.75(d, 2H), J=8.7 Hz), 7.88(d, 2H, J=8.7 Hz), 8.55(s, 1H) | 361 |
| 38 | 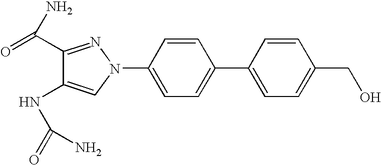 | (CD₃OD/DMSO-d₆(4:1)): δ 4.57(s, 2H), 7.37(d, 2H, J=8.1 Hz), 7.60(d, 2H, J=8.1 Hz), 7.733(d, 2H, J=8.6 Hz), 7.83(d, 2H, J=8.6 Hz), 8.52(s, 1H) | 352 |
| 39 | 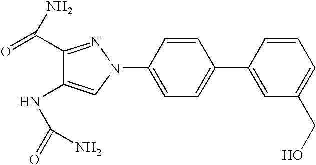 | (CD₃OD/DMSO-d₆(4:1)): δ 4.57(s, 2H), 7.22-7.28(m, 1H), 7.30-2.38(m, 1H), 7.43-7.50(m, 1H), 7.54-7.58(m, 1H), 7.67 (d, 2H, J=8.7 Hz), 7.80 (d, 2H, J=8.7 Hz), 8.46 (s, 1H) | |
| 40 | 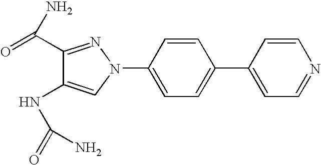 | (CD₃OD/DMSO-d₆(4:1)): δ 7.72(d, 2H, J=5.3 Hz), 7.91(d, 2H, J=8.5 Hz), 8.00(d, 2H, J=8.6 Hz), 8.56-8.64(s, 3H) | |
| 41 | 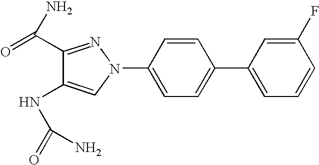 | (300 MHz, DMSO-d₆): δ6.51(br s, 2H), 7.24(m, 1H), 7.47-7.70(m, 6H), 7.88(s, 1H), 7.90(d, J₁=1.2 Hz, 1H), 8.22(m, 1H), 8.68(s, 1H), 8.71 (s, 1H) | 340.17 |
| 42 | 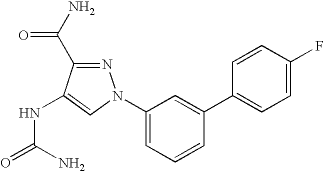 | (300 MHz, DMSO-d₆): δ6.51(br s, 2H), 7.28(t, J=8.9 Hz, 2H), 7.48-7.65(m, 3H), 7.77-7.91 (m, 4H), 8.16(m, 1H), 8.68(s, 2H) | 340.17 |
| 43 | 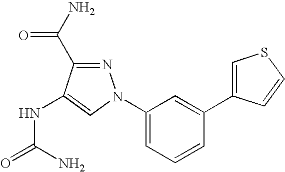 | (300 MHz, DMSO-d₆): δ6.52(br s, 2H), 7.50-7.60(m, 2H), 7.66-7.72 (m, 3H), 7.82(d, J=7.5 Hz, 1H), 7.87(s, 1H), 8.06(m, 1H), 8.23(s, 1H), 8.67(m, 2H) | 328.0867 |
| 44 | 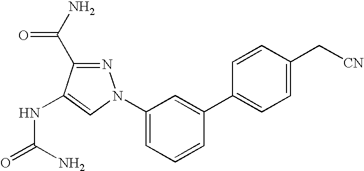 | (300 MHz, DMSO-d₆): δ4.10(s, 2H), 6.52(br s, 2H), 7.48(d, J=7.90 Hz, 2H), 7.53(s, 1H), 7.62(m, 2H), 7.860-7.90(m, 4H), 8.19(s, 1H), 8.67(m, 2H) | 361.1406 |
| 45 | 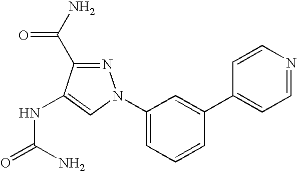 | (300 MHz, DMSO-d₆): δ6.52(br s, 2H), 7.55(br s, 1H), 7.64(t, J=8.02 Hz, 1H), 7.76(d, J=8.33 Hz, 1H), 7.84 (m, 2H), 7.90(s, 1H), 7.99(d, J=8.02 Hz, 1H), 8.32(s, 1H), 8.67 (m, 3H), 8.73(s, 1H) | 323.1270 |
| 46 | 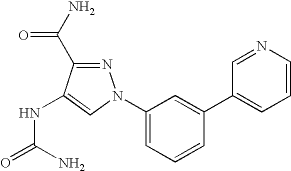 | (300 MHz, DMSO-d₆): δ6.51(m, 2H), 7.50-7.58 (m, 2H), 7.61(t, J=7.95 Hz, 1H), 7.70(m, 1H), 7.88-7.95(m, 2H), 8.20 (m, 1H), 8.29(t, J=1.91 Hz, 1H), 8.62(m, 1H), 8.68(s, 1H), 8.72(s, 1H), 9.02(d, J=2.22 Hz, 1H) | 323.1 |
| 47 | 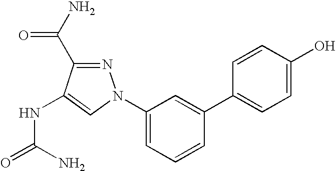 | | 338.1 |
| 48 | 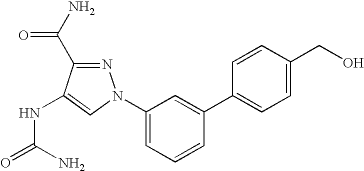 | (300 MHz, DMSO-d₆): δ4.55(d, J=4.03 Hz, 2H), 5.24(t, J=4.03 Hz, 1H), 6.51(s, 2H), 7.43(d, J=8.05 Hz, 2H), 7.60-7.65(m, 3H), 7.75(d, J=8.05 Hz, 2H), 7.83 (m, 1H), 7.88(s, 1H), 8.18(t, J=1.81 Hz, 1H), 8.67(s, 1H), 8.69(s, 1H) | 352.1 |
| 49 | 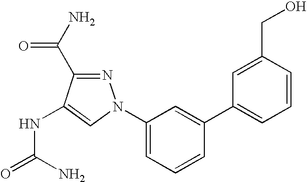 | (300 MHz, DMSO-d₆): δ4.59(d, J=5.64 Hz, 2H), 5.26(t, J=5.64 Hz,1H), 6.52(sq, 2H), 7.30-7.75(m, 7H), 7.85(m, 2H), 8.14(m, 1H), 8.66 (s, 1H), 8.69(s, 1H) | 352.1380 |
| 50 | 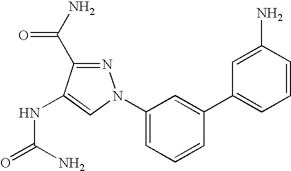 | (300 MHz, DMSO-d₆): δ5.19(s, 2H), 6.52(s, 2H), 6.60(d, J=7.85 Hz, 1H), 6.90-6.97(m, 2H), 7.12(t, J=7.75 Hz, 1H), 7.48-7.60(m, 3H), 7.80 (d, J=7.05 Hz, 1H), 7.85(s, 1H), 8.03(s, 1H), 8.62(s, 1H), 8.69(s, 1H) | 337.1400 |
| 51 | 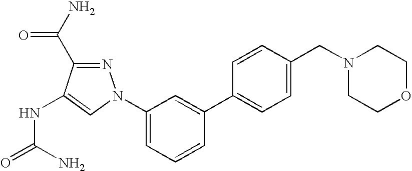 | (300 MHz, DMSO-d₆): δ2.37(m, 4H), 3.50(s, 2H), 3.57(m, 4H), 6.52 (s, 2H), 7.41(d, J=8.06 Hz, 2H), 7.50-7.90(m, 7H), 8.17(s, 1H), 8.67 (s, 1H), 8.69(s, 1H) | 421.1987 |
| 52 | 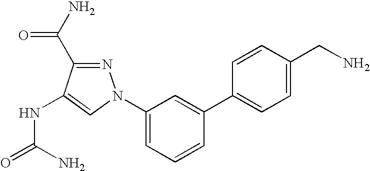 | (300 MHz, DMSO-d₆): δ3.80(s, 2H), 6.50(br s, 2H), 7.50(d, J=8.1 Hz, 2H), 7.51-7.64(m, 3H), 7.73(d, J=8.3 Hz, 2H), 7.82(d, 1H), 7.88(1, 1H), 8.17(m, 1H), 8.60 (s, 1H), 8.69(s, 1H) | 351.1595 |
| 53 | 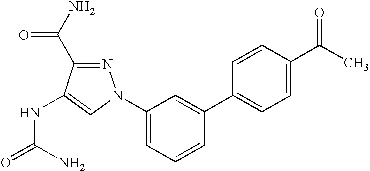 | (300 MHz, DMSO-d₆): δ2.62(s, 3H), 6.52(br s, 2H), 7.51-7.79(m, 3H), 7.89(d, J=6.3 Hz, 2H), 7.94(d, J=8.1 Hz, 2H), 8.05(d, J=8.1 Hz, 2H), 8.26(1, 1H), 8.69(s, 1H), 8.71(s, 1H) | 364.1435 |
| 54 | 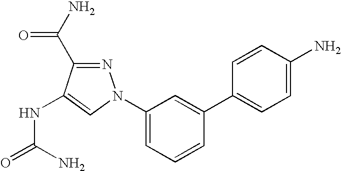 | (300 MHz, DMSO-d₆): δ6.52(br s, 2H), 6.66(d, 2H), 7.39-7.60(m, 6H), 7.86(s, 1H), 7.98(s, 1H), 8.57(s, 1HH), 8.69 (s, 1H) | 337.1 |
| 55 | 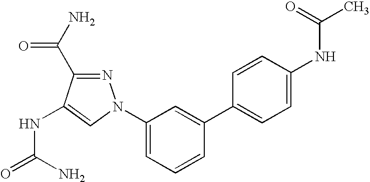 | (300 MHz, DMSO-d₆): δ2.06(s, 1H), 6.52(br s, 2H), 7.50-7.95(m, 9H), 8.32(s, 1H), 8.66(s, 1H), 8.68(s, 1H), 10.06 (s, 1H) | 379.1 |
| 56 | 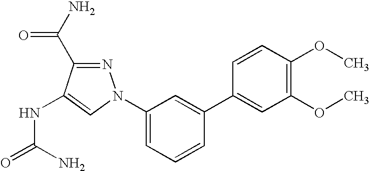 | (300 MHz, DMSO-d₆): δ3.80(s, 3H), 3.87(s, 3H), 6.52(br s, 2H), 7.04 (d, J=9 Hz, 1H), 7.31 (m, 2H), 7.56(m, 3H), 7.78(d, J=9 Hz, 1H), 7.86(s, 1HH), 8.12(s, 1H), 8.66(s, 1H), 8.69 (m, 1H) | 382.1550 |
| 57 | 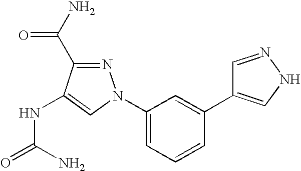 | | 312.1 |
| 58 | 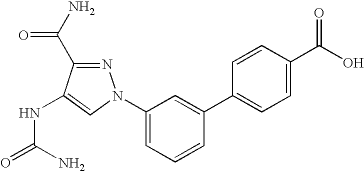 | (300 MHz, DMSO-d₆): δ6.52(br s, 2H), 7.54(s, 1H), 7.62(m, 1H), 7.69 (d, J=7.8 Hz, 1H), 7.87-8.00(m, 4H), 8.06(d, J=8.7 Hz, 2H), 8.26(s, 1H), 8.68(s, 1H), 8.71 (s, 1H) | 366.1190 |
| 59 | 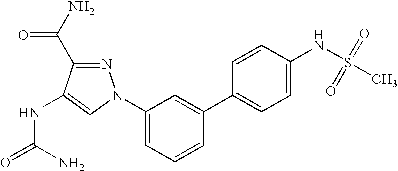 | (300 MHz, DMSO-d₆): δ3.091(s, 3H), 6.51(s, 2H), 7.31(m, 2H), 7.56 (m, 3H), 7.77(m, 2H), 7.82(dt, J=7.60, 1.74 Hz, 1H), 7.86(s, 1H), 8.15(t, J=1.71 Hz, 1H), 8.66(s, 1H), 8.68(s, 1H), 9.90(m, 1H) | 415.1226 |
| 60 | 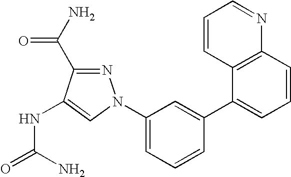 | (300 MHz, DMSO-d₆): δ6.48(m, 2H), 7.40-8.00 (m, 8H), 8.07(m, 2H), 8.21(m, 1H), 8.68(m, 2H), 8.95(dd, J=4.23, 1.61 Hz, 1H) | 373.1 |
| 61 | 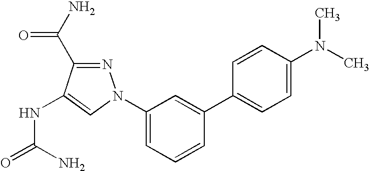 | (300 MHz, DMSO-d₆): δ2.95(s, 6H), 6.47(m, 2H), 6.81(d, J=8.86 Hz, 2H), 7.52(m, 3H), 7.64 (d, J=8.86 Hz, 2H), 7.71(m, 1H), 7.86(m, 1H), 8.08(m, 1H), 8.63 (s, 1H), 8.68(s, 1H) | 365.2 |
| 62 | 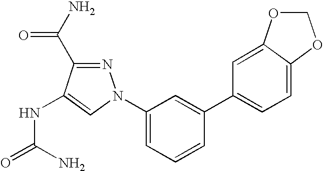 | (300 MHz, DMSO-d₆): δ6.07(s, 2H), 6.51(m, 2H), 7.02(d, J=8.06 Hz, 1H), 7.28(dd, J=8.16, 1.91 Hz, 1H), 7.40(d, J=1.81 Hz, 1H), 7.52 (m, 3H), 7.80(m, 1H), 7.88(m, 1H), 8.11(t, J=1.71 Hz, 1H), 8.67 (m, 2H) | 366.1 |
| 63 | 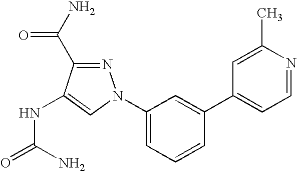 | (300 MHz, DMSO-d₆): δ2.55(s, 3H), 6.51(m, 2H), 7.55(m, 1H), 7.62 (m, 2H), 7.72(m, 2H), 7.89(m, 1H), 7.97(d, J=8.05 Hz, 1H), 8.28(t, J=1.91 Hz, 1H), 8.53 (d, J=5.24 Hz, 1H), 8.68(m, 1H), 8.71(s, 1H) | 337.1 |
| 64 | 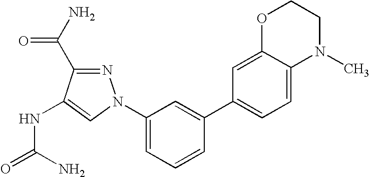 | (300 MHz, DMSO-d₆): δ2.86(m, 3H), 3.26(m, 2H), 4.25(m, 2H), 6.48 (m, 2H), 6.77(d, J=8.46 Hz, 1H), 7.12(m, 1H), 7.23(d, J=6.65 Hz, 1H), 7.51(m, 3HH), 7.70 (d, J=6.65 Hz, 1H), 7.88(m, 1H), 8.04(m, 1H), 8.63(s, 1H), 8.69(s, 1H) | 393.1663 |
| 65 | 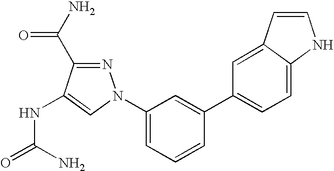 | (300 MHz, DMSO-d₆): δ6.51(m, 3H), 7.39(m, 1H), 7.50(m, 4H), 7.63 (m, 1HH), 7.76(m, 1H), 7.88(m, 1H), 7.95(m, 1H), 8.17(t, J=1.81 Hz, 1H), 8.65(s, 1H), 8.69(s, 1HH), 11.17(s, 1H) | 361.1403 |
| 66 | 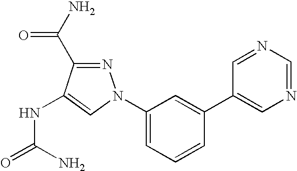 | (300 MHz, DMSO-d₆): δ6.51(m, 2H), 7.53(m, 1H), 7.65(*t, J=7.985 Hz, 1H), 7.78(m, 1H), 7.91 (m, 2H), 8.01(m, 1H), 8.40(m, 1H), 8.66(m, 1H), 8.76(s, 1H), 9.22(s, 1H), 9.28(s, 1H) | 324.1 |
| 67 | 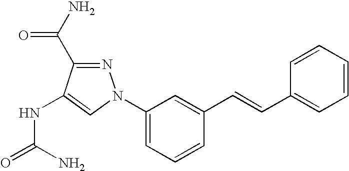 | (300 MHz, DMSO-d₆): δ6.51(m, 2H), 7.26-7.69 (m, 1)H), 7.79(m, 2H), 8.14(m, 1H), 8.643(s, 1H), 8.69(s, 1H) | 348.1 |
| 68 | 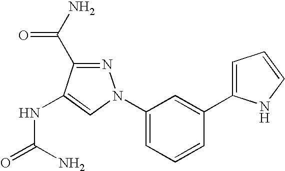 | | 311.1256 |
| 69 | 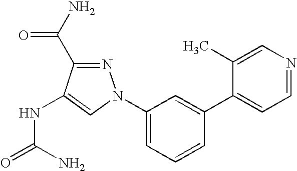 | (400 MHz, DMSO-d₆): δ2.36(s, 3H), 6.50(m, 2H), 7.41(m, 1H), 7.52 (s, 1H), 7.64(t, J=8.19 Hz, 1H), 7.72(d, J=5.37 Hz, 1H), 7.82(s, 1H), 8.00(dd, J=2.28, 0.94 Hz, 1H), 8.02(d, J=0.81 Hz, 1H), 8.66 (m, 2H), 8.69(d, J=5.37 Hz, 1H), 8.76(s, 1H) | 337.1422 |
| 70 | 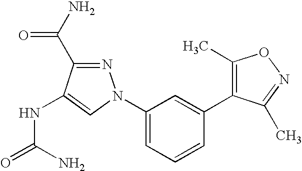 | | 341.1290 |
| 71 | 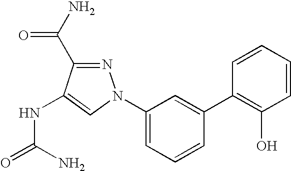 | (400 MHz, DMSO-d₆): δ6.48(m, 2H), 6.90(m, 2H), 7.16(m, 1H), 7.32 (m, 1H), 7.48(m, 3H), 7.76(m, 2H), 7.99(m, 1H), 8.56(s, 1H), 8.67 (m, 1H), 9.62(s, 1H) | 338.1247 |
| 72 | 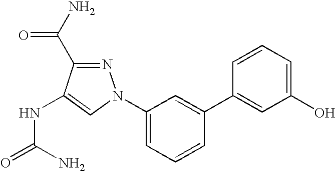 | (400 MHz, DMSO-d₆): δ6.50(m, 2H), 6.79(m, 1H), 7.10(m, 1H), 7.16 (d, J=8.32 Hz, 1H), 7.27(t, J=7.79 Hz, 1H) 7.49(s, 1H), 7.54(m, 3H), 7.82(m, 2H), 8.08 (m, J=1.34 Hz, 1H), 8.63(s, 1H), 8.67(s, 1H), 9.54(m, 1H) | 338.1 |
| 73 | 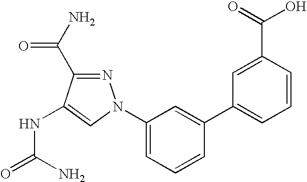 | (300 MHz, DMSO-d₆): δ6.48(m, 2H), 7.51(m, 1H), 7.63(m, 3H), 7.9 (m, 2H), 7.98(d, J=7.65 Hz, 1H), 8.04(d, J=7.85 Hz, 1H), 8.23 (dt, J=15.71, 1.61 Hz, 2H), 8.68(s, 1HJ), 8.70(s, 1H), 13.21(m, 1H) | 366.1 |
| 74 | 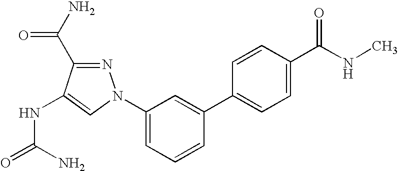 | (300 MHz, DMSO-d₆): δ2.80(m, 3H), 6.51(s, 2H), 7.52(m, 1H), 7.59(t, J=7.85 Hz, 1H), 7.69 (d, J=8.05 Hz, 1H), 7.84-8.03(m, 6H), 8.24 (t, J=1.81 Hz, 1H), 8.52 (d, J=4.63 Hz, 1H), 8.69(s, 1H), 8.70(s, 1H) | 379.1543 |
| 75 | 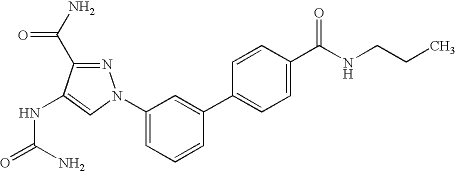 | (300 MHz, DMSO-d₆): δ0.90(m, 3H), 1.54(m, 2H) 3.23(m, 2H), 6.50 (m, 2H), 7.52(m, 1H), 7.59(t, J=7.95 Hz, 1H), 7.69(m, 1H), 7.84-8.01 (m, 5H), 8.12(s, 1H), 8.24(t, J=1.81 Hz, 1H), 8.53(t, J=5.54 Hz, 1H), 8.69(s, 1H), 8.70(s, 1H) | 407.1821 |
| 76 | 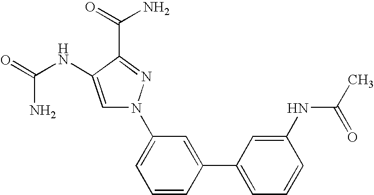 | (300 MHz, DMSO-d₆): δ2.05(m, J=2.42 Hz, 3H), 6.51(m, 2H), 7.30-7.70(m, 6H), 7.85(m, 3H), 8.09(m, 1H), 8.64 (s, 1H), 8.68(s, 1H), 10.02(s, 1H) | 379.1559 |
| 77 | 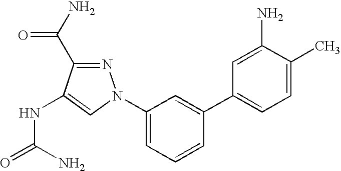 | (300 MHz, DMSO-d₆): δ2.22(s, 3H), 6.50(m, 2H), 7.16-7.36(m, 3H), 7.56(m, 3H), 7.84(m, 2H), 8.06(m, J=1.41 Hz, 1H), 8.63(s, 1H), 8.69 (s, 1H) | 351.1540 |
| 78 | 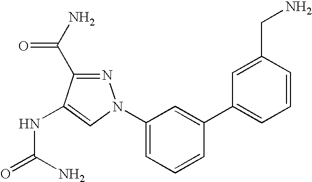 | (300 MHz, DMSO-d₆): δ4.05(s, 2H), 6.50(m, 2H), 7.37-7.69(m, 5H), 7.75(d, 1H), 7.81-7.96 (m, 3H), 8.17(s, 1H), 8.65(s, 1H), 8.69(s, 1H) | 351.1 |
| 79 | 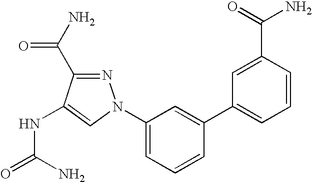 | (300 MHz, DMSO-d₆): δ6.48(m, 2H), 7.41-7.74 (m, 5H), 7.85-7.97(m, 4H), 8.13(m, 1H), 8.23 (m, 2H), 8.69(m, 2H) | 365.1 |
| 80 | 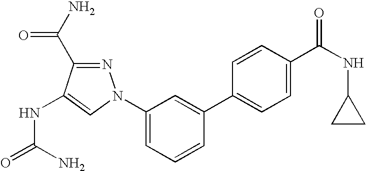 | (300 MHz, DMSO-d₆): δ0.50-0.80(m, 4H), 2.87 (m, 1H), 6.51(m, 2H), 7.49-7.71(m, 4H), 7.83-8.00(m, J=9.46 Hz, 5H), 8.22(m, 1H), 8.52(m, 1H), 8.68(s, 1H), 8.69(s, 1H) | 405.1 |
| 81 | 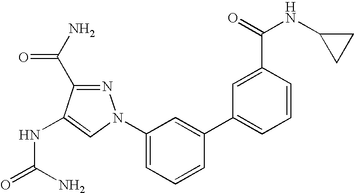 | (300 MHz, DMSO-d₆): δ0.52-0.80(m, 4H), 2.87 (m, 1H), 6.44(m, 2H), 7.47-7.74(m, 4H), 7.83-7.96(m, 4H), 8.19 (m, 2H), 8.57(d, J=4.03 Hz, 1H), 8.69(m, 2H) | 405.1 |
| 82 | 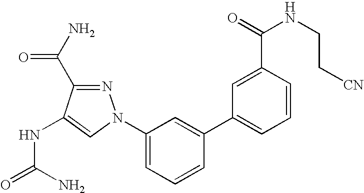 | (300 MHz, DMSO-d₆): δ2.80(t, J=6.44 Hz, 2H), 3.53(t, J=6.44 Hz, 2H), 6.48(m, 2H), 7.49-7.63 (m, 4H), 7.80-8.03(m, 4H), 8.21(m, 2H), 8.69 (s, 2H), 8.99(s, 1H) | 418.1 |
| 83 | 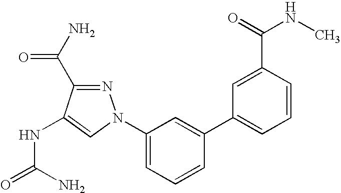 | (400 MHz, DMSO-d₆): δ2.80(d, J=4.56 Hz, 3H), 6.53(m, 2H), 7.50-7.70(m, 4H), 7.81-8.00 (m, 4H), 8.17(d, J=1.61 Hz, 1H), 8.20(t, J=1.88 Hz, 1H), 8.56(d, J=4.56 Hz, 1H), 8.68(s, 2H) | 379.1498 |
| 84 | 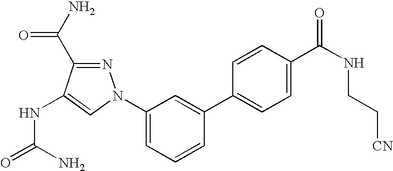 | (300 MHz, DMSO-d₆): δ2.78(t, J=6.76 Hz, 2H), 3.51(t, J=6.76 Hz, 2H), 6.48(s, 2H), 7.52(s, 1H), 7.61(t, J=7.64 Hz, 1H), 7.70(d, J=8.17 Hz, 1H), 7.82-8.02(m, 6H), 8.23(m, 1H), 8.67(s, 1H), 8.68(s, 1H), 8.91(m, 1H) | 418.1623 |
| 85 | 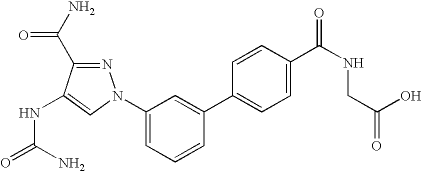 | (400 MHz, DMSO-d₆): δ3.93(d, J=5.91 Hz, 2H), 6.51(m, 2H), 7.52 (m, 1H)(, 7.59(t, J=7.92 Hz, 1H), 7.70(d, J=8.59 Hz, 1H)(, 7.84-8.012(m, J=17.99 Hz, 7H), 8.25(m, 1H), 8.67 (m, 1H), 8.69(s, 1H), 8.91(t, J=5.91 Hz, 1H) | 423.1412 |
| 86 | 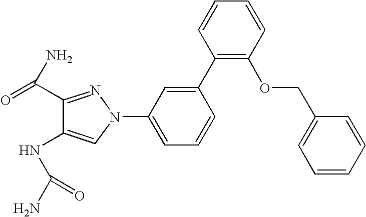 | (400 MHz, DMSO-d₆): δ5.12(s, 2H), 6.50(m, 2H), 7.06(td, J=7.45, 0.94 Hz, 1H), 7.19-7.29 (m, 4H), 7.32-7.52(m, 7H), 7.75(s, 1H), 7.78 (m, 1H), 8.09(t, J=1.75 Hz, 1H), 8.59(s, 1H), 8.68(s, 1H) | 428.1694 |
| 87 | 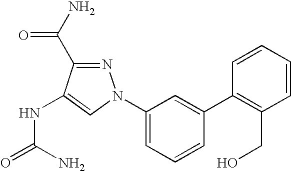 | (400 MHz, DMSO-d₆): δ4.40(s, 2H), 6.50(m, 2H), 7.30-7.60(m, 7H), 7.78(s, 1H), 7.85(m, 1H), 7.91(m, 1H), 8.61 (s, 1H), 8.68(s, 1H) | 352.1430 |
| 88 | 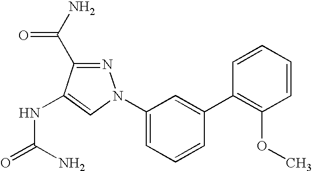 | (300 MHz, DMSO-d₆): δ3.78(s, 3H), 6.50(m, 2H), 7.05(t, J=7.55 Hz, 1H), 7.14(d, J=8.66 Hz, 1H), 7.33-7.58(m, 5H), 7.79(m, 2H), 7.94(m, 1H), 8.59(m, 1H), 8.68 (m, 1HH) | 352.1425 |
| 89 | 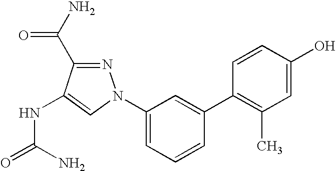 | (400 MHz, DMSO-d₆): δ2.17(s, 3H), 6.47(m, 2H), 6.65(dd, J=8.59, 2.69 Hz, 1H), 6.69(d, J=2.42 Hz, 1H), 7.06 (d, J=8.32 Hz, 1H), 7.22(d, J=8.32 Hz, 1H), 7.45(m, 1H), 7.48(t, J=7.92 Hz, 1H), 7.78 (m, 3H), 8.58(s, 1H), 8.66(s, 1H), 9.38(m, 1H) | 352.1403 |
| 90 | 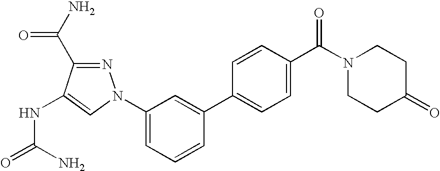 | (300 MHz, DMSO-d₆): δ3.84(m, 8H), 6.52(m, 2H), 7.55-7.70(m, 5H), 7.90(m, 4H), 8.24(t, J=1.81 Hz, 1H), 8.69(s, 1H), 8.70(s, 1H) | 447.1 |
| 91 | 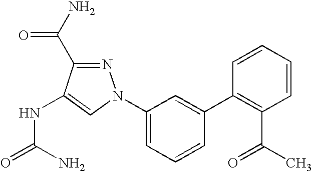 | (300 MHz, DMSO-d₆): δ2.24(s, 3H), 6.44(m, 2H), 7.19(d, J=7.45 Hz, 1H), 7.47-7.70(m, 6H), 7.82-7.90(m, 3H), 8.63 (s, 1H), 8.68(s, 1H) | 364.1 |
| 92 | 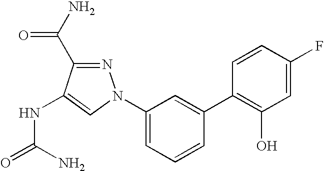 | (300 MHz, DMSO-d₆): δ6.49(s, 2H), 6.66(m, 2H), 7.35(m, 1H), 7.49 (m, 3H), 7.77(m, 2H), 7.97(m, 1H), 8.56(s, 1H), 8.68(s, 1H) | 356.1190 |
| 93 | 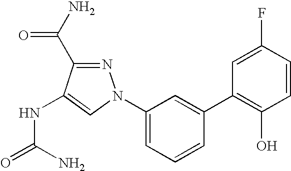 | (400 MHz, DMSO-d₆): δ6.49(s, 2H), 6.93(dd, J=8.86, 5.10 Hz, 1H), 7.02(td, J=8.53, 3.09 Hz, 1H), 7.21(dd, J=9.67, 3.22 Hz, 1H), 7.50(m, 4H), 7.80(m, 2H), 8.01(m, 1H), 8.59 (s, 1H), 8.67(s, 1H) | 356.1196 |
| 94 | 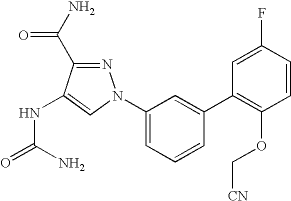 | (300 MHz, DMSO-d₆): δ5.15(s, 2H), 6.50(s, 2H), 7.30-7.60(m, 6H), 7.77(s, 1H), 7.87(d, J=8.46 Hz, 1H), 7.99 (m, 1H), 8.63(s, 1H), 8.67(s, 1H) | 395.1 |
| 95 | 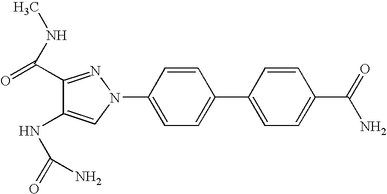 | (400 MHz, DMSO-d₆): δ2.63(d, J=4.30 Hz, 3H), 7.24(m, 1H), 7.37 (s, 1H), 7.55(s, 1H), 7.75-8.15(m, 10H), 8.64(s, 1H), 8.70(s, 1H) | 379.10 |

### Examples 96-141

### 4-[(aminocarbonyl)amino]-1-(4-arylphenyl)-1H-pyrazole-3-carboxamides

4-[(aminocarbonyl)amino]-1-(4-iodophenyl)-1H-pyrazole-3-carboxamide (0.22 g, 0.6 mmol, prepared according to the process of Example 214), Pd[PPh₃]₄ (50 mg), and 0.9 mmol of an arylboronic acid were added to a flask and placed under vacuum then refilled with N₂. The vacuum/N₂ cycle was repeated twice. DMF (3 mL) and 0.9 mL of 2M Cs₂CO₃ solution were injected under N₂. (The DMF and Cs₂CO₃ solutions were bubbled with N₂ for 15 min before addition). The suspension was heated and stirred at 80° C. overnight. Most of liquids were removed under reduced pressure. The solid was triturated with water (80 mL), filtered, triturated with CH₃OH (5-10 mL) for 1-3 h, filtered and dried to give the compounds shown in the following table.

| | | | MS (ES⁺) |
|---|---|---|---|
| Ex. | Name and Structure | ¹H NMR | (M + 1) |
| 96 | 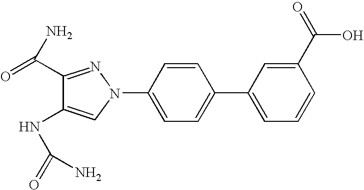 | (CD₃OD)/DMSO-d₆ (4:1): δ7.48(t, 1H, J=8.7 Hz), 7.72-7.86(m, 3H), 7.87-8.00(m, 3H), 8.20(s, 1H), 8.58(s, 1H) | 366 |
| 97 | 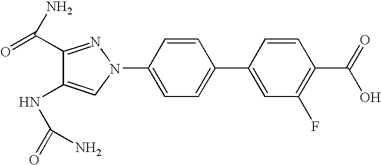 | (CD₃OD)/DMSO-d₆ (4:1): δ7.63-7.71(m, 2H), 7.83-8.02 (m, 5H), 8.62(s, 1H) | 384 |
| 98 | 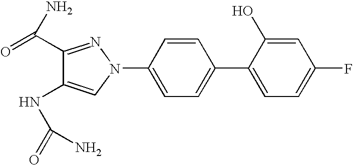 | DMSO-d₆: δ 6.51(br s, 2H), 6.63-6.79(m, 2H), 7.32(t, 1H, J=7.8 Hz), 7.50(br s, 1H), 7.65(d, 2H, J=8.7 Hz), 7.78(br s, 1H), 7.88(d, 2H, J=8.7 Hz), 8.58(s, 1H), 8.68 (s, 1H), 10.20(br s, 1H) | 356 |
| 99 | 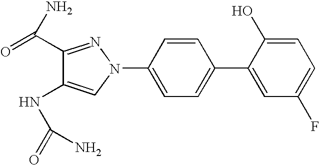 | DMSO-d₆: δ 6.51(br s, 2H), 6.91-7.154(m, 2H), 7.35(dd, 1H, J₁=2.8 Hz, J₂= 6.8 Hz), 7.51(br s, 1H), 7.71(d, 2H, J=8.6 Hz), 7.78(br s, 1H), 7.88(d, 2H, J=8.6 Hz), 8.58 (s, 1H), 8.68(s, 1H), 10.20 (br s, 1H) | 356 |
| 100 | 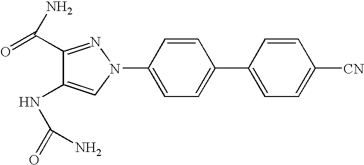 | DMSO-d₆: δ 6.53(br s, 2H), 7.55(br s, 1H), 7.83(br s, 1H), 7.88-8.00(m, 6H), 8.02 (d, 2H, J=8.7 Hz), 8.64(s, 1H), 8.70(s, 1H) | 347 |
| 101 | 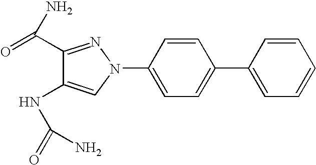 | DMSO-d₆: δ 6.53(br s, 2H), 7.32-7.56(m, 4H), 7.72(d, 2H, J=7.4 Hz), 7.80(d, 3H, J=8.6 Hz), 7.96(d, 2H, J=8.7 Hz), 8.62(s, 1H), 8.69(s, 1H) | 322 |
| 102 | 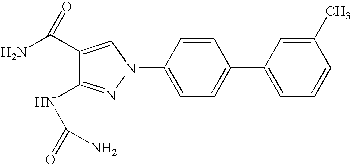 | | 336 |
| 103 | 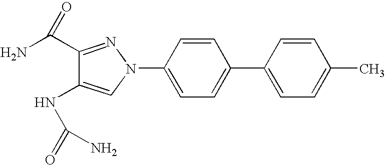 | | 336 |
| 104 | 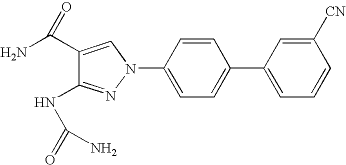 | | 347 |
| 105 | 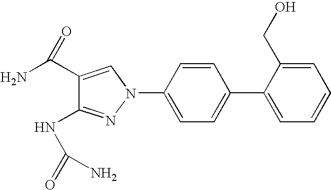 | | 352 |
| 106 | 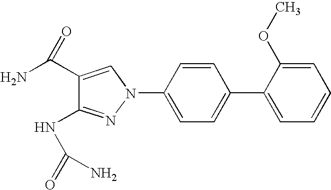 | | 352 |
| 107 | 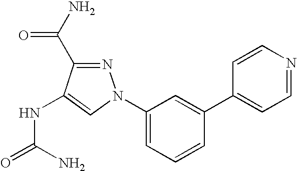 | | 352 |
| 108 | 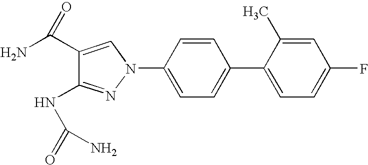 | | 354 |
| 109 | 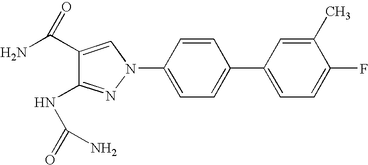 | | 354 |
| 110 | 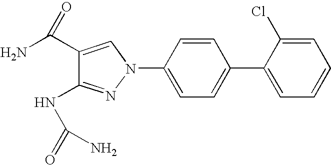 | | 356 |
| 111 | 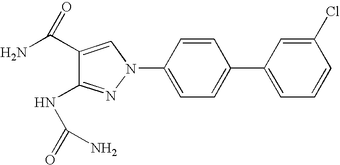 | | 356 |
| 112 | 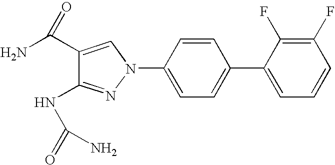 | | 358 |
| 113 | 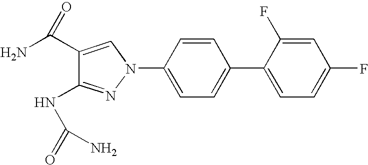 | | 358 |
| 114 | 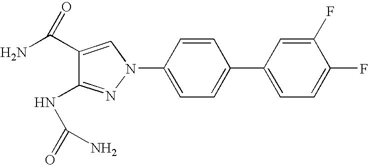 | | 358 |
| 115 | 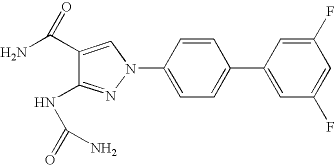 | | 358 |
| 116 | 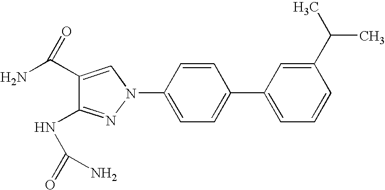 | | 364 |
| 117 | 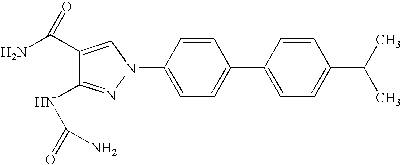 | | 364 |
| 118 | 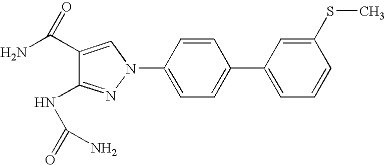 | | 368 |
| 119 | 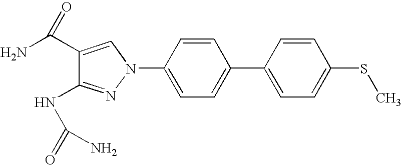 | | 368 |
| 120 | 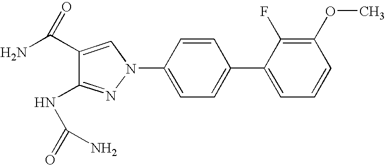 | | 370 |
| 121 | 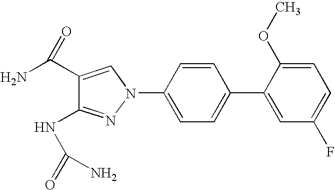 | | 370 |
| 122 | 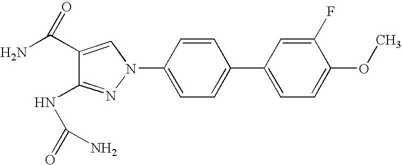 | | 370 |
| 123 | 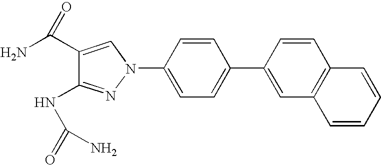 | | 372 |
| 124 | 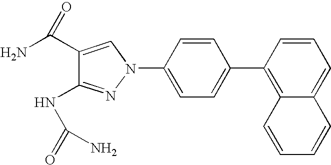 | | 372 |
| 125 | 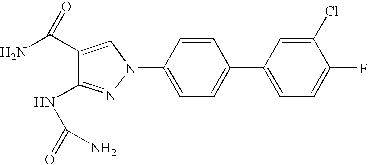 | | 374 |
| 126 | 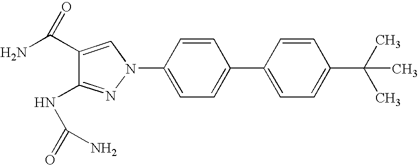 | | 378 |
| 127 | 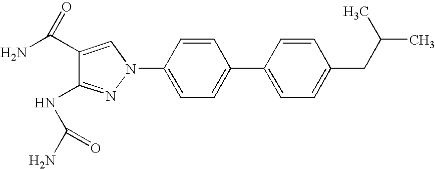 | | 378 |
| 128 | 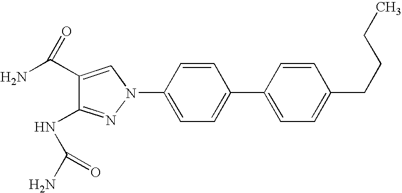 | | 378 |
| 129 | 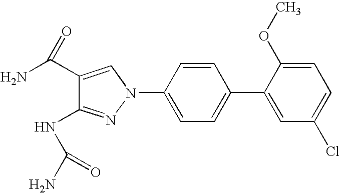 | | 386 |
| 130 | 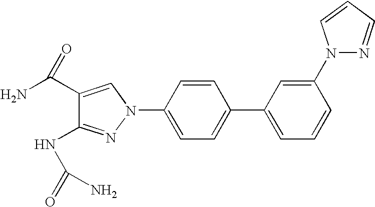 | | 388 |
| 131 | 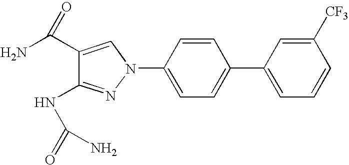 | | 390 |
| 132 | 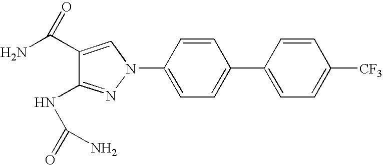 | | 390 |
| 133 | 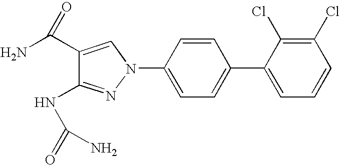 | | 390 |
| 134 | 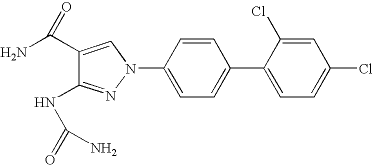 | | 390 |
| 135 | 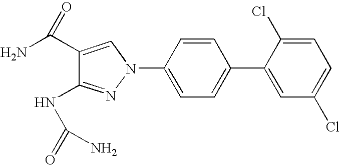 | | 390 |
| 136 | 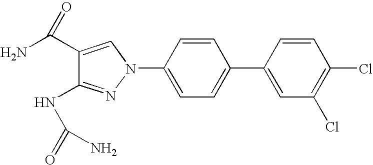 | | 390 |
| 137 | 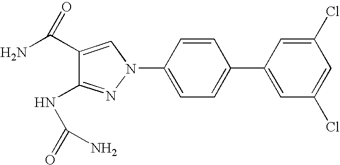 | | 390 |
| 138 | 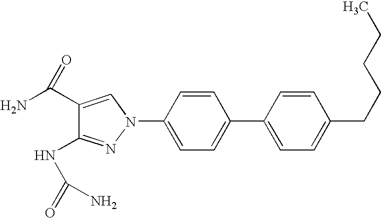 | | 392 |
| 139 | 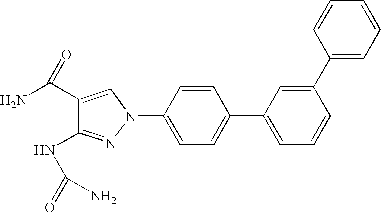 | | 398 |
| 140 | 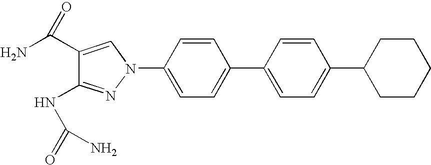 | | 404 |
| 141 | 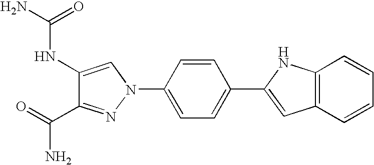 | | |

### Example 142

### 4′-{3-(aminocarbonyl)-4-[(aminocarbonyl)amino]-1H-pyrazol-1-yl}-1,1′-biphenyl-4-carboxylic acid

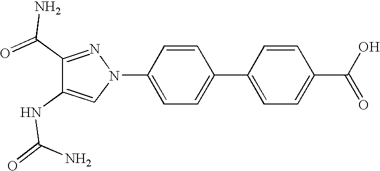

4-[(aminocarbonyl)amino]-1-(4-bromophenyl)-1H-pyrazole-3-carboxamide (Example 19, 0.19 g, 0.6 mmol), Pd[PPh₃]₄(50 mg), and 0.9 mmol of an arylboronic acid were added to a flask and placed under vacuum then refilled with N₂. The vacuum/N₂ cycle was repeated twice. DMF (3 mL) and 0.9 mL of 2M Cs₂CO₃ solution were injected under N₂. (The DMF and Cs₂CO₃ solutions were bubbled with N₂ for 15 min before addition). The suspension was heated and stirred at 80° C. overnight. Most of liquids were removed under reduced pressure. The solid was triturated with 1 N HCl (80 mL), filtered, triturated with a saturated bicarbonate solution (5-10 mL) for 1-3 h, filtered and dried. ¹H NMR (CD₃OD)/DMSO-d₆(4:1): δ 7.18-7.25 (m, 4H), 7.26-7.42 (m, 4H), 8.00 (s, 1H). ESI mass spectrum for C₁₈H₁₆N₅O₄⁺: 366 (M+1).

### Examples 143-164

### 4-[(aminocarbonyl)amino]-1-{4′-[(R,R′-amino)carbonyl]-1,1′-biphenyl-4-yl}-1H-pyrazole-3-carboxamides and alkyl 4′-{3-(aminocarbonyl)-4-[(aminocarbonyl)amino]-1H-pyrazol-1-yl}-1,1′-biphenyl-4-carboxylates

An arylcarboxylic acid (0.03 mmol), prepared according to Example 10142, was combined with 0.06 mmol of an amine, or an alcohol, HBTU (BF₄) (0.06 mmol), N,N-dimethylethylamine (0.06 mL), and DMSO (1.0 mL). The mixture was stirred overnight, then most of liquids were stripped off. The residue was triturated in 50 mL of water for 6 h and filtered. The solid was triturated with ethanol (5 mL) for 3 h and filtered, then dried under reduced pressure.

| | | | MS(ES⁺) |
|---|---|---|---|
| Ex. | Name and Structure | ¹H NMR | (M + 1) |
| 143 | 4-[(aminocarbonyl)amino]-1-{4′- | (CD₃OD)/DMSO-d₆ | 421 |
| | [(diethylamino)carbonyl]-1,1′-biphenyl-4-yl}- | (4:1): δ 1.00-1.22 (m, | |
| | 1H-pyrazole-3-carboxamide | 6H), 3.21 (br s, 2H), | |
| | 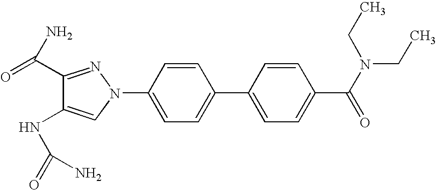 | 3.44 (br s, 2H), 7.39 (d, 2H, J=8.2 Hz), 7.62-7.80 (m, 4H), 7.89 (d, 2H, J=8.6 Hz), 8.5 (s, 1H) | |
| 144 | ethyl 4′-{3-(aminocarbonyl)-4- | (CD₃OD): δ 1.42 (t, | 394 |
| | [(aminocarbonyl)amino]-1H-pyrazol-1-yl}-1,1′- | 3H, J=7.1 Hz), 4.42 | |
| | biphenyl-3-carboxylate | (q, 2H, J=7.1 Hz), | |
| | 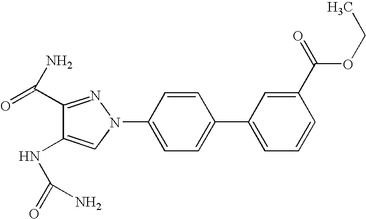 | 7.58 (t, 1H, J=7.8 Hz), 7.82(d, 2H, J=8.7 Hz), 7.91 (s, 1H), 7.95 (d, 2H, J=8.7 Hz), 8.25 (d, 1H, J=8.7 Hz), 8.30 (s, 1H), 8.57 (s, 1H) | |
| 145 | 4-[(aminocarbonyl)amino]-1-{4′- | (CD₃OD)/DMSO-d₆ | 379 |
| | [(methylamino)carbonyl]-1,1′-biphenyl-4-yl}-1H- | (4:1): δ 2.80 (s, 3H), | |
| | pyrazole-3-carboxamide | 7.70 (d, 2H, J=8.3 | |
| | 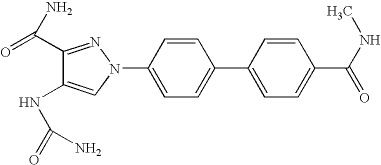 | Hz), 7.75 (d, 2H, J=8.7 Hz), 7.82 (d, 2H, J=8.3 Hz), 7.87 (d, 2H, J=8.7 Hz), 8.48 (s, 1H) | |
| 146 | 4-[(aminocarbonyl)amino]-1-{4′- | (CD₃OD)/DMSO-d₆ | 407 |
| | [(propylamino)carbonyl]-1,1′-biphenyl-4-yl}-1H- | (4:1): δ 0.95 (t, 3H, | |
| | pyrazole-3-carboxamide | J=7.3 Hz), 1.55 | |
| | 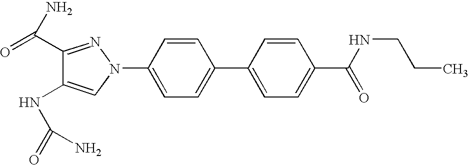 | (sextet, 2H, J=7.2 Hz), 3.22 (t, 2H, J=7.2 Hz), 7.73 (d, 2H, J=8.3 Hz), 7.90 (d, 2H, J=8.7 Hz), 7.83-7.93 (m, 4H), 8.48 (s, 1H) | |
| 147 | ethyl 4′-{3-(aminocarbonyl)-4- | DMSO-d₆: δ 1.38 (t, | 394 |
| | [(aminocarbonyl)amino]-1H-pyrazol-1-yl}-1,1′- | 3H, J=7.1 Hz), 4.38 | |
| | biphenyl-4-carboxylate | (q, 2H, J=7.1 Hz), | |
| | 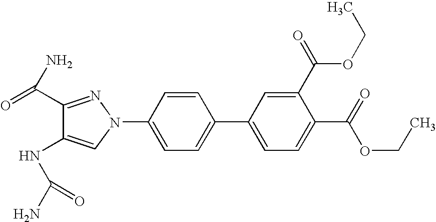 | 6.50 (br s, 2H), 7.52 (br s, 1H), 7.78-7.93 (m, 5H), 7.99-8.09 (m, 4H), 8.62 (s, 1H), 8.73 (s, 1H) | |
| 148 | 4-[(aminocarbonyl)amino]-1-{3′- | CD₃OD: δ 1.29 (d, | 407 |
| | [(isopropylamino)carbonyl]-1,1′-biphenyl-4-yl}-1H- | 6H, J = 6.6 Hz), 4.38 | |
| | pyrazole-3-carboxamide | (septet, 1H, J=6.6 | |
| | 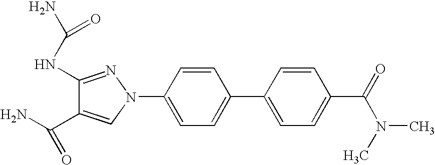 | Hz), 7.48 (t, 1H, J=7.8 Hz), 7.78-7.88 (m, 4H), 7.85 (d, 2H, J=8.8 Hz), 8.12 (t, 1H, J=1.6 Hz), 8.58 (s, 1H) | |
| 149 | 4-[(aminocarbonyl)amino]-1-[4′-(aminocarbonyl)-1,1′- | (CD₃OD)/DMSO-d₆ | 365 |
| | biphenyl-4-yl]-1H-pyrazole-3-carboxamide | (4:1): δ 7.72 (d, 2H, | |
| | 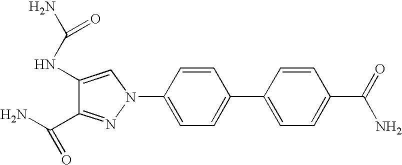 | J=8.3 Hz), 7.76 (d, 2H, J=8.6 Hz), 7.83-7.93 (m, 4H), 8.51 (s, 1H) | |
| 150 | 4-[(aminocarbonyl)amino]-1-(3′-fluoro-4′-{[(4- | (CD₃OD)/DMSO-d₆ | 503 |
| | methoxybenzyl) amino]carbonyl}-1,1′-biphenyl-4-yl)- | (4:1): δ 3.65 (s, 3H), | |
| | 1H-pyrazole-3-carboxamide | 4.40 (s, 2H), 6.81 (d, | |
| | 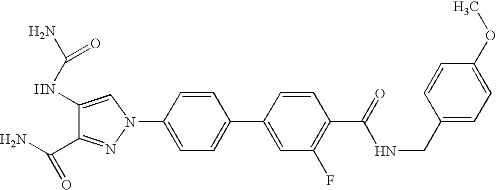 | 2H, J=8.7 Hz), 7.21 (d, 2H, J=8.7 Hz), 7.46-7.57 (m, 2H), 7.70 (d, 1H, J=8.0 Hz), 7.76 (d, 2H, J=8.9 Hz), 7.88 (d, 2H, J=8.9 Hz), 8.52 (s, 1H) | |
| 151 | 4-[(aminocarbonyl)amino]-1-{4′- | DMSO-d₆: δ 4.48 (d, | 473 |
| | [(benzylamino)carbonyl]-3′-fluoro-1,1′-biphenyl-4-yl}- | 2H, J=5.9 Hz), 6.52 | |
| | 1H-pyrazole-3-carboxamide | (br s, 2H), 7.20-7.30 | |
| | 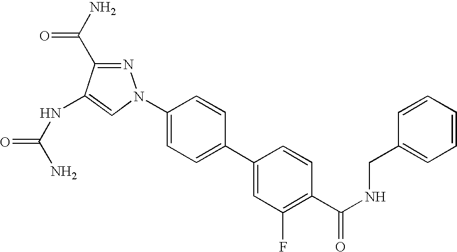 | (m, 1H), 7.35 (d, 4H, J=4.4 Hz), 7.55 (br s, 1H), 7.65-7.78 (m, 3H), 7.84 (br s, 1H), 7.91 (d, 2H, J=8.8 Hz), 8.05 (d, 2H, J=8.8 Hz), 8.64 (s, 1H), 8.69 (s, 1H) | |
| 152 | 4-[(aminocarbonyl)amino]-1-{3′-fluoro-4′- | DMSO-d₆: δ 2.79(d, | 397 |
| | [(methylamino)carbonyl]-1,1′-biphenyl-4-yl}-1H- | 3H, J=4.5 Hz), 6.52 | |
| | pyrazole-3-carboxamide | (br s, 2H), 7.45(br s, | |
| | 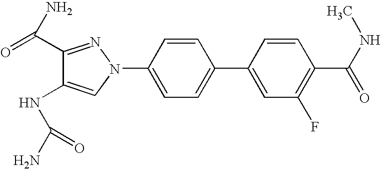 | 1H), 7.62-7.78 (m, 3H), 7.84(br s, 1H), 7.90(d, 2H, J=8.8 Hz),8.00(d, 2H, J=8.8Hz), 8.20-8.29 (m, 1H), 8.63(s, 1H), 8.68(s, 1H) | |
| 153 | 4-[(aminocarbonyl)amino]-1-(3′-fluoro-4′-{[(3- | DMSO-d₆: δ 3.75(s, | |
| | methoxybenzyl)amino]carbonyl}-1,1′-biphenyl-4-yl)- | 3H), 4.47(d, 2H, | |
| | 1H-pyrazole-3-carboxamide | J=5.7Hz), 6.52(br s, | |
| | 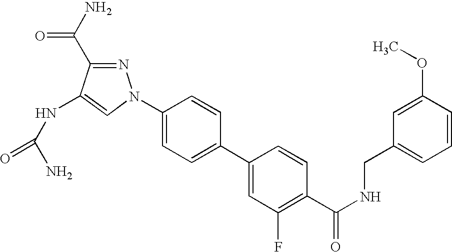 | 2H), 6.71(d, 1H, J=8.1Hz), 6.81(d, 2H, J=2.4Hz), 7.25 (t, 1H, J=8.0Hz), 7.55(br s, 1H), 7.60-7.80(m, 3H), 7.85(br s, 1H), 7.90(d, 2H, J=8.7Hz), 8.05(d, 2H, J=8.7Hz), 8.64 (s, 1H), 8.69(s, 1H), 8.88(t, 1H, J=5.7Hz) | |
| 154 | 4-[(aminocarbonyl)amino]-1-(3′-fluoro-4′-{[(2- | DMSO-d₆: δ 3.92(s, | |
| | methoxybenzyl)amino]carbonyl}-1,1′-biphenyl-4-yl)- | 3H), 4.48(d, 2H, | |
| | 1H-pyrazole-3-carboxamide | J=5.7 Hz), 6.55(br s, | |
| | 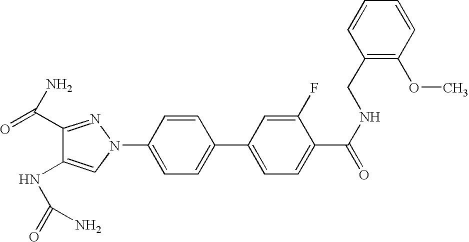 | 2H), 6.95(t, 1H, J=7.5Hz), 7.00(d, 1H, J=8.5Hz), 7.20-7.33(m, 2H), 7.55 (br s, 1H), 7.65-7.82 (m, 3H), 7.85(br s, 1H), 7.92(d, 2H, J=8.7Hz), 8.05(d, 2H, J=8.7Hz), 8.60-8.75(m, 3H) | |
| 155 | 4-[(aminocarbonyl)amino]-1-(3′-fluoro-4′-{[(4- | DMSO-d₆: δ 4.45(d, | 491 |
| | fluorobenzyl)amino]carbonyl}-1,1′-biphenyl-4-yl)-1H- | 2H, J=5.7Hz), 6.51 | |
| | pyrazole-3-carboxamide | (br s, 2H), 7.24(t, 2H, | |
| | 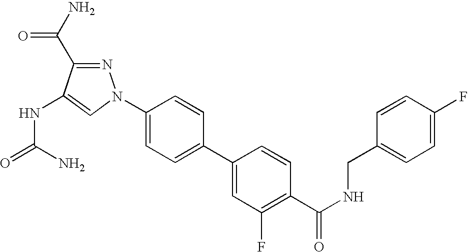 | J=8.9 Hz), 7.32-7.45 (m, 2H), 7.53(br s, 1H), 7.61-7.80(m, 3H), 7.85(br s, 1H), 7.93(d, 2H, J=8.8 Hz), 8.03(d, 2H, J=8.8Hz), 8.62(s, 1H), 8.68(s, 1H), 9.90(t, 1H, J=5.7 Hz) | |
| 156 | 4-[(aminocarbonyl)amino]-1-(3′-fluoro-4′-{[(3- | DMSO-d₆: δ 4.49(d, | 491 |
| | fluorobenzyl)amino]carbonyl}-1,1′-biphenyl-4-yl)-1H- | 2H, J=5.7 Hz), 6.52 | |
| | pyrazole-3-carboxamide | (br s, 2H), 7.02-7.22 | |
| | 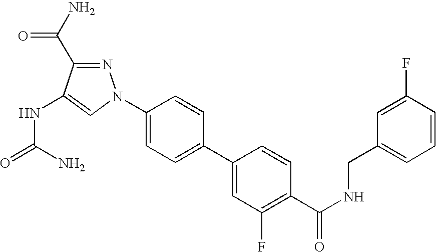 | (m, 3H), 7.32-7.45 (m, 1H), 7.53(br s, 1H), 7.65-7.80(m, 3H), 7.85(br s, 1H), 7.93(d, 2H, J=8.8 Hz), 8.03(d, 2H, J=8.8Hz), 8.64(s, 1H), 8.70(s, 1H), 9.95(t, 1H, J=5.7Hz) | |
| 157 | 4-[(aminocarbonyl)amino]-1-(3′-fluoro-4′-{[(2- | DMSO-d₆: δ 4.53(d, | 491 |
| | fluorobenzyl)amino]carbonyl}-1,1′-biphenyl-4-yl)-1H- | 2H, J=5.6Hz), 6.52 | |
| | pyrazole-3-carboxamide | (br s, 2H), 7.12-7.45 | |
| | 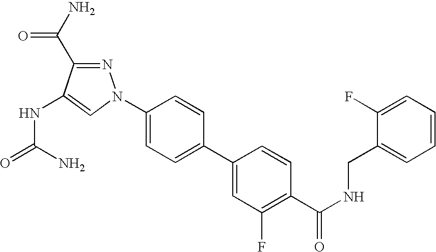 | (m, 4H), 7.55(br s, 1H), 7.65-7.80(m, 3H), 7.85(br s, 1H), 7.94(d, 2H, J=8.8 Hz), 8.05(d, 2H, J=8.8Hz), 8.64(s, 1H), 8.69(s, 1H), 9.89(t, 1H, J=5.6Hz) | |
| 158 | 4-[(aminocarbonyl)amino]-1-(3′-fluoro-4′-{[(4- | DMSO-d₆: δ 2.80(s, | 505 |
| | fluorobenzyl)(methyl)amino]carbonyl}-1,1′-biphenyl- | 3H), 4.73(s, 2H), | |
| | 4-yl)-1H-pyrazole-3-carboxamide | 6.52(br s, 2H), 7.12- | |
| | 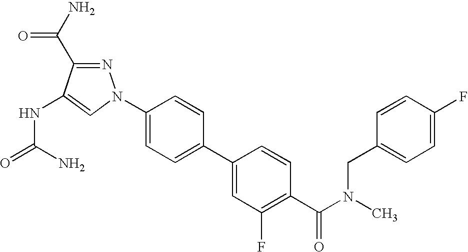 | 7.28(m, 3H), 7.34-7.44(m, 1H), 7.54(t, 2H, J=7.2Hz), 7.62-7.78(m, 2H), 7.79-7.95(m, 3H), 7.95-8.08(m, 2H), 8.64(s, 1H), 8.69(s, 1H) | |
| 159 | 4-[(aminocarbonyl)amino]-1-{3′-fluoro-4′-[(1,2,3,4- | DMSO-d₆: δ 1.73- | 505 |
| | tetrahydronaphthalen-1-ylamino)carbonyl]-1,1′- | 2.05(m, 4H), 2.67- | |
| | biphenyl-4-yl}-1H-pyrazole-3-carboxamide | 2.81(m, 2H), 5.17- | |
| | 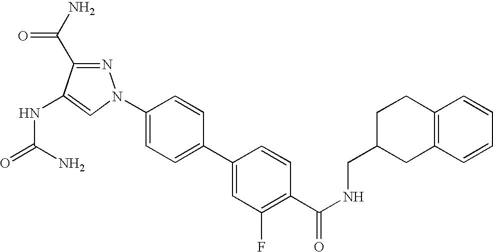 | 5.28(m, 1H), 6.52(br s, 2H), 7.06-7.20(m, 3H), 7.26-7.33(m, 1H), 7.56(br s, 1H), 7.63-7.73(m, 3H), 7.83(br s, 1H), 7.86-7.93(m, 2H), 8.01(d, 2H, J=8.8Hz), 8.62 (s, 1H), 8.64-8.69(m, 2H) | |
| 160 | 4-[(aminocarbonyl)amino]-1-(4′-{[[2- | (CD₃OD): δ 2.98(s, | 468 |
| | (dimethylamino)ethyl](methyl)amino]carbonyl}-3′- | 6H), 3.07(s, 3H), | |
| | fluoro-1,1′-biphenyl-4-yl)-1H-pyrazole-3- | 3.45(t, 2H, J=6.2 | |
| | carboxamide | Hz), 3.94(t, 2H, | |
| | 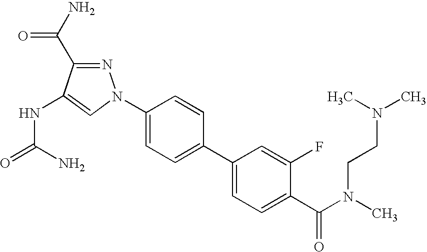 | J=6.2Hz), 7.52-7.70 (m, 3H), 7.84(d, 2H, J=8.6Hz), 7.96(d, 2H, J=8.6Hz), 8.59 (s, 1H) | |
| 161 | 4-[(aminocarbonyl)amino]-1-[5′-fluoro-2′-(2- | CD₃OD: δ 3.38-3.66 | 482 |
| | morpholin-4-yl-2-oxoethoxy)-1,1′-biphenyl-4-yl]-1H- | (m, 8H), 4.79(s, 2H), | |
| | pyrazole-3-carboxamide | 7.04-7.10(m, 2H), | |
| | 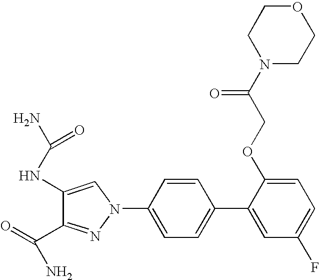 | 7.12-7.20(m, 1H), 7.72(d, 2H, J=8.7 Hz), 7.89(d, 2H, J=8.7Hz), 8.58(s, 1H) | |
| 162 | 2-fluorobenzyl 4′-{3-(aminocarbonyl)-4- | DMSO-d₆: δ 5.38(s, | 492 |
| | [(aminocarbonyl)amino]-1H-pyrazol-1-yl}-3-fluoro- | 2H), 6.53(br s, 2H), | |
| | 1,1′-biphenyl-4-carboxylate | 7.20-7.63(m, 5H), | |
| | 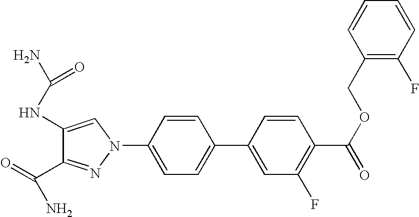 | 7.72-7.90(m, 3H), 7.90-8.08(m, 5H), 8.63(s, 1H), 8.69(s, 1H) | |
| | 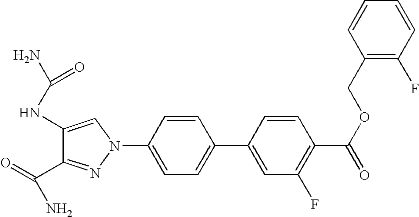 | | |
| 163 | 3-fluorobenzyl 4′-{3-(aminocarbonyl)-4- | DMSO-d₆: δ 5.38(s, | 492 |
| | [(aminocarbonyl)amino]-1H-pyrazol-1-yl}-3-fluoro- | 2H), 6.52(br s, 2H), | |
| | 1,1′-biphenyl-4-carboxylate | 7.18(t, 1H, J=8.9 | |
| | 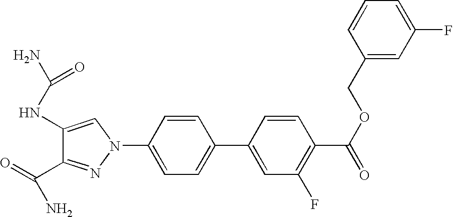 | Hz), 7.33(d, 2H, J=8.9Hz), 7.40-7.60 (m, 2H), 7.70-7.90 (m, 3H), 7.90-8.10 (m, 5H), 8.62(s, 1H), 8.67(s, 1H) | |
| 164 | 4-fluorobenzyl 4′-{3-(aminocarbonyl)-4- | DMSO-d₆: δ 5.38(s, | |
| | [(aminocarbonyl)amino]-1H-pyrazol-1-yl}-3-fluoro- | 2H), 6.53(br s, 2H), | |
| | 1,1′-biphenyl-4-carboxylate | 7.25(t, 2H, J=8.9 | |
| | 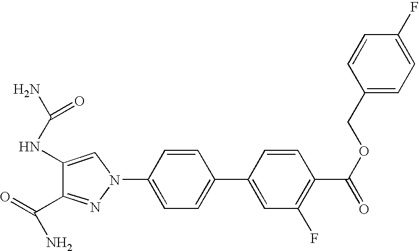 | Hz), 7.48-7.60(m, 3H), 7.71-7.89(m, 3H), 7.90-8.08(m, 5H), 8.63(s, 1H), 8.68(s, 1H) | |

### Example 165

### 4′-{3-(aminocarbonyl)-4-[(aminocarbonyl)amino]-1H-pyrazol-1-yl}-1,1′-biphenyl-3-yl)methyl acetate

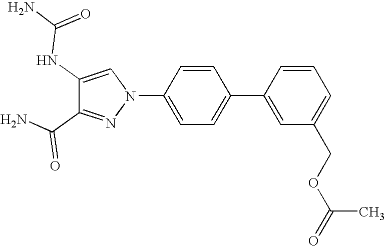

4-[(aminocarbonyl)amino]-1-[4′-(hydroxymethyl)-1,1′-biphenyl-4-yl]-1H-pyrazole-3-carboxamide (Example 38, 0.105 g, 0.03 mmol) was combined with 0.06 mmol of an acetic acid, HBTU (BF₄) (0.06 mmol), N,N-dimethylethylamine (0.06 mL), and DMSO (1.0 mL). The mixture was stirred overnight, then most of the liquids were stripped off. The residue was triturated in 25 mL of water for 6 h and filtered. The solid was triturated with ethanol (5 mL) for 3 h and filtered. The product was dried under reduced pressure. ¹H NMR ((CD₃OD)/DMSO-d₆ (4:1)): δ 2.10 (s, 3H), 5.20 (s, 2H), 7.38 (d, 1H, J=7.4 Hz), 7.47 (t, 1H, J=7.6 Hz), 7.64 (d, 1H, J=7.5 Hz), 7.68 (s, 1H), 7.78 (d, 2H, J=8.7 Hz), 7.93 (d, 2H, J=8.7 Hz), 8.57 (s, 1H). ESI mass spectrum for C₂₀H₂₀N₅O₄⁺: 394 (M+1).

### Examples 166-182

### Alkylation of Phenols

A mixture of phenol prepared according to Examples 21-141 (0.03 mmol), K₂CO₃ (0.062 g, 0.045 mmol), and 0.045 mmol of a bromomethyl-derivative (bromoacetonitrile, bromomethylpyridines, bromoacetic acid and its esters or amide) in a DMF/acetone (1:1) mixture (3 mL) was stirred at rt for 48 h. Solvents were removed, and water (30 mL) was added. The suspension was sonicated for 30 min and triturated for 3 h to give the desired product after drying.

| | | ¹H NMR (DMSO-d₆, unless | MS(ES⁺) |
|---|---|---|---|
| Ex. | Name and Structure | indicated otherwise) | (M + 1) |
| 166 | 4-[(aminocarbonyl)amino]-1-[3′-(cyanomethoxy)- | δ 5.28(s, 2H), 6.50(br s, | 377 |
| | 1,1′-biphenyl-4-yl]-1H-pyrazole-3-carboxamide | 2H), 7.08(d, 1H, J=7.2Hz), | |
| | 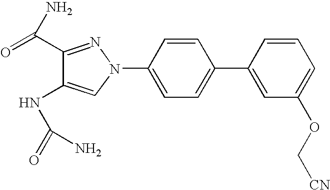 | 7.39-7.50(m, 3H), 7.54(br s, 1H), 7.67(d, 2H, J=8.7Hz), 7.78-7.90(m, 3H), 8.62(s, 1H),8.92(s, 1H) | |
| 167 | 4-[(aminocarbonyl)amino]-1-[2′-(cyanomethoxy)- | δ 5.18(s, 2H), 6.50(br s, | 377 |
| | 1,1′-biphenyl-4-yl]-1H-pyrazole-3-carboxamide | 2H), 7.88(t, 1H, J=7.5Hz), | |
| | 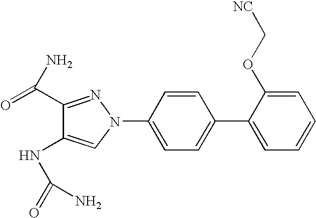 | 7.26(d, 1H, J=8.1Hz), 7.42 (d, 2H, J=7.4Hz), 7.52(br s, 1H), 7.59(d, 2H, J=8.3Hz), 7.79(br s, 1H), 7.93(d, 2H, J=8.3Hz), 8.62(s, 1H), 8.72 (s, 1H) | |
| 168 | 4-[(aminocarbonyl)amino]-1-[3′-(pyridin-2- | (CD₃OD)/DMSO-d₆(4:1): δ | 429 |
| | ylmethoxy)-1,1′-biphenyl-4-yl]-1H-pyrazole-3- | 5.28(s, 2H), 6.93(dd, 1H, | |
| | carboxamide | J₁=6.3, 1.7Hz), 7.15-7.33(m, | |
| | 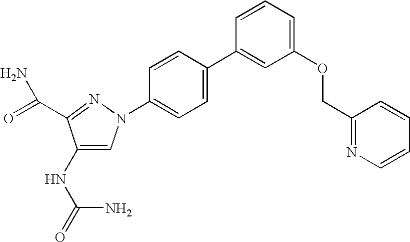 | 4H), 7.53(d, 1H, J=7.9Hz), 7.65(d, 2H, J=8.8Hz), 7.75 (dd, 1H, J₁=6.2, 1.7Hz), 7.80 (d, 2H, J=8.8Hz), 8.40-8.50 (m, 2H) | |
| 169 | 4-[(aminocarbonyl)amino]-1-[3′-(1-cyanoethoxy)- | δ 1.71(d, 3H, J=6.7Hz), 5.60 | 391 |
| | 1,1′-biphenyl-4-yl]-1H-pyrazole-3-carboxamide | (q, 1H, J=6.7Hz), 6.51(br s, | |
| | 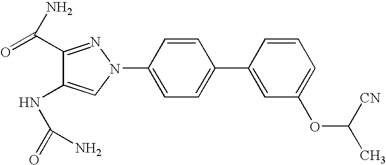 | 2H), 7.15-7.23(m, 1H), 7.40-7.48(m, 3H), 7.53(br s, 1H), 7.75-7.87(m, 3H), 7.98(d, 2H, J=8.7Hz), 8.61(s, 1H), 8.68(s, 1H) | |
| 170 | 4-[(aminocarbonyl)amino]-1-[3′-(pyridin-3- | δ 5.23(s, 2H), 6.55(br s, | 429 |
| | ylmethoxy)-1,1′-biphenyl-4-yl]-1H-pyrazole-3- | 2H), 7.10(dd, 1H, J=5.8, 2.2 | |
| | carboxamide | Hz), 7.30-7.48(m, 4H), 7.53 | |
| | 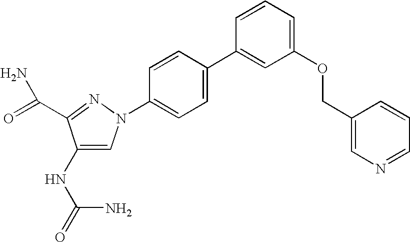 | (br s, 1H), 7.78-7.86(m, 3H), 7.82(d, 1H, J=7.9Hz), 7.95 (d, 2H, J=8.8Hz), 8.55(d, 1H, J=3.5Hz), 8.61(s, 1H), 8.65-8.75(m, 2H) | |
| 171 | 4-[(aminocarbonyl)amino]-1-[3′-(pyridin-4- | δ 5.58(s, 2H), 6.54(br s, | 429 |
| | ylmethoxy)-1,1′-biphenyl-4-yl]-1H-pyrazole-3- | 2H), 7.10(dd, 1H, J₁6.4, 1.7 | |
| | carboxamide | Hz), 7.30-7.45(m, 3H), 7.48 | |
| | 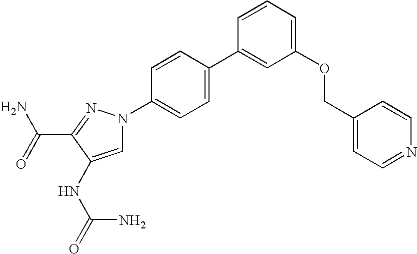 | (d, 2H, J=7.9Hz), 7.53(br s, 1H), 7.78-7.86(m, 3H), 7.93 (d, 2H, J=8.8Hz), 8.55-8.65 (m, 3H), 8.68(s, 1H) | |
| 172 | 4-[(aminocarbonyl)amino]-1-[2′-(cyanomethoxy)-5′- | δ 5.27(s, 2H), 6.53(br s, | 395 |
| | fluoro-1,1′-biphenyl-4-yl]-1H-pyrazole-3- | 2H), 6.25-7.38(m, 3H), 7.52 | |
| | carboxamide | (brs, 1H), 7.65(d, 2H, J=8.7 | |
| | 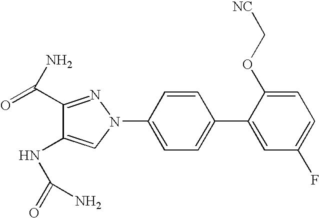 | Hz), 7.82(br s, 1H), 7.95 (d, 2H, J=8.7Hz), 8.62(s, 1H), 8.69(s, 1H) | |
| 173 | 4-[(aminocarbonyl)amino]-1-[2′-(cyanomethoxy)-4′- | δ 5.24(s, 2H), 6.53(br s, | 395 |
| | fluoro-1,1′-biphenyl-4-yl]-1H-pyrazole-3- | 2H), 7.04(dt, 1H, J=6.1, 2.1 | |
| | carboxamide | Hz), 7.25(dd, 1H, J=8.9, 2.1 | |
| | 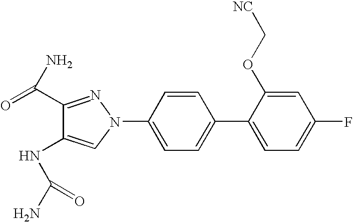 | Hz), 7.40-7.62(m, 4H), 7.78 (br s, 1H), 7.93(d, 2H, J=8.7 Hz), 8.60(s, 1H), 8.69(s, 1H) | |
| 174 | tert-butyl [(4′-{3-(aminocarbonyl)-4- | (CD₃OD): δ 1.51(s, 9H), 4.42 | 452 |
| | [(aminocarbonyl)amino]-1H-pyrazol-1-yl}-1,1′- | (s, 2H), 6.94(d, 1H, J=9.0 | |
| | biphenyl-3-yl)oxy]acetate | Hz), 7.18-7.22(m, 1H), 7.23- | |
| | 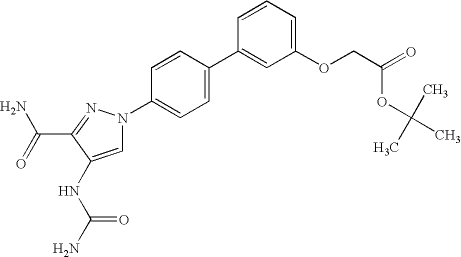 | 7.34(m, 1H), 7.37-7.42(m, 1H), 7.78(d, 2H, J=8.6Hz), 7.95(d, 2H, J=8.6Hz), 8.58 (s, 1H) | |
| 175 | 4-[(aminocarbonyl)amino]-1-[3′-(2-amino-2- | δ 4.48(s, 2H), 6.55(br s, | 395 |
| | oxoethoxy)-1,1′-biphenyl-4-yl]-1H-pyrazole-3- | 2H), 6.99(d, 1H J=7.7Hz), | |
| | carboxamide | 7.28-7.46(m, 4H), 7.56(d, | |
| | 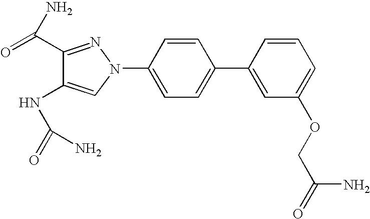 | 2H, J=7.4 Hz), 7.77-7.86(m, 3H), 7.99(d, 2H, J=8.7Hz), 8.62(s, 1H), 8.72(s, 1H) | |
| 176 | 4-[(aminocarbonyl)amino]-1-[2′-(cyanomethoxy)- | δ 2.90(s, 2H)6.50(s, 2H) | 377.1 |
| | 1,1′-biphenyl-3-yl]-1H-pyrazole-3-carboxamide | 7.20(t, 1H)7.30(d, 1H) | |
| | 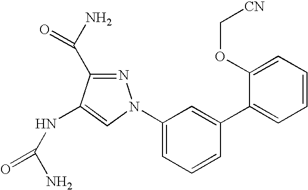 | 7.40-7.60(m, 5H)7.75(s, 1 H)7.83(d, 1H)8.00(m, 1H) 8.65(s, 1H)8.69(s, 1H) | |
| 177 | 4-[(aminocarbonyl)amino]-1-[4′-(cyanomethoxy)- | δ 5.21(m, 2H)6.50(m, 2H) | 377.1 |
| | 1,1′-biphenyl-3-yl]-1H-pyrazole-3-carboxamide | 7.19(m, J=9.06Hz, 2H) | |
| | 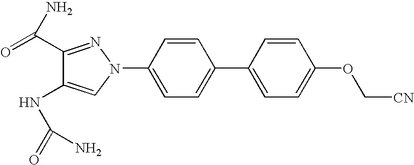 | 7.47-7.66(m, 3H)7.77-7.94 (m, 4H)8.17(m, 1H)8.65 (m, 2H) | |
| 178 | 4-[(aminocarbonyl)amino]-1-[3′-(cyanomethoxy)- | δ 5.28(s, 2H)6.50(m, 2H) | 377.1326 |
| | 1,1′-biphenyl-3-yl]-1H-pyrazole-3-carboxamide | 7.13(dd, J=4.13, 2.92Hz, 1 | |
| | 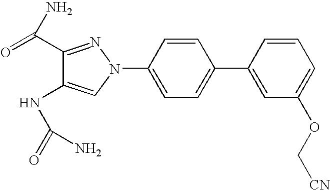 | H)7.45-7.70(m, 6H)7.89 (m, 2H)8.19(t, J=1.81Hz, 1 H)8.69(s, 2H) | |
| 179 | 4-[(aminocarbonyl)amino]-1-[3′-(cyanomethoxy)-3- | δ 5.30(s, 2H), 6.55(br s, | 395 |
| | fluoro-1,1′-biphenyl-4-yl]-1H-pyrazole-3- | 2H), 7.10-7.18(m, 1H), 7.45- | |
| | carboxamide | 7.62(m, 4H), 7.72-7.90(m, | |
| | 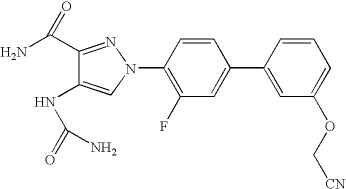 | 3H), 8.05(t, 1H, J=11.9.7 Hz), 8.51(s, 1H), 8.71(s, 1H) | |
| 180 | 4-[(aminocarbonyl)amino]-1-[2′-(1-cyanoethoxy)-5′- | δ 1.58(d, 3H, J=6.7Hz), 5.34 | 409 |
| | fluoro-1,1′-biphenyl-4-yl]-1H-pyrazole-3- | (q, 1H, J=6.7Hz), 6.53(br s, | |
| | carboxamide | 2H), 7.24-7.40(m, 3H), 7.53 | |
| | 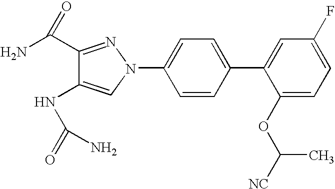 | (br s, 1H), 7.65(d, 2H, J=8.7 Hz), 7.82(br s, 1H), 7.96(d, 2H, J=8.7Hz), 8.63(s, 1H), 8.69(s, 1H) | |
| 181 | 4-[(aminocarbonyl)amino]-1-{3′-[2-(dimethylamino)- | δ 2.85(s, 3H), 3.03(s, 3H), | |
| | 2-oxoethoxy]-1,1′-biphenyl-4-yl}-1H-pyrazole-3- | 4.90(s, 2H), 6.53(br s, 2H), | |
| | carboxamide | 6.78-6.92(m, 1H), 7.20-7.42 | |
| | 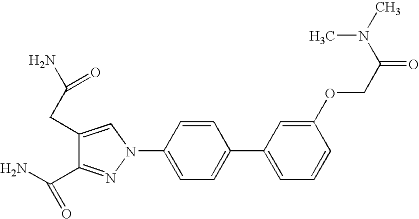 | (m, 3H), 7.53(br s, 1H), 7.78 (d, 3H, J=8.4Hz), 7.93(d, 2H, J=8.4Hz), 8.63(s, 1H), 8.69(s, 1H) | |
| 182 | tert-butyl [(4′-{3-(aminocarbonyl)-4- | δ 1.40(s, 9H), 5.39(s, 2H), | |
| | [(aminocarbonyl)amino]-1H-pyrazol-1-yl}-5-fluoro- | 6.52(br s, 2H), 6.98-7.17(m, | |
| | 1,1′-biphenyl-2-yl)oxy]acetate | 1H), 7.20-7.32(m, 1H), 7.25 | |
| | 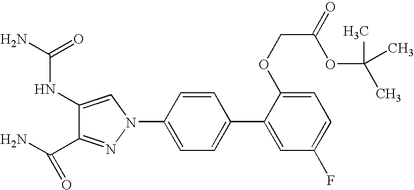 | (dd, 1H, J=6.2, 3.0Hz), 7.53 (br s, 1H), 7.75(d, 2H, J=8.8 Hz), 7.80(s, 1H), 7.94(d, 2H, J=8.8Hz), 8.60(s, 1H), 8.69 (s, 1H) | |

### Example 183

### 4-[(aminocarbonyl)amino]-1-[4′-(aminocarbonyl)-3′-fluoro-1,1′-biphenyl-4-yl]-1H-pyrazole-3-carboxamide

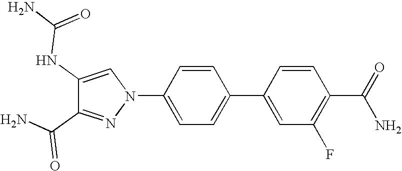

A mixture of 4-[(aminocarbonyl)amino]-1-(3′-fluoro-4′-{[(4-methoxybenzyl)amino]carbonyl}-1,1′-biphenyl-4-yl)-1H-pyrazole-3-carboxamide (Example 150, 0.104 g, 0.2 mmol), 2.5 mL of TFA, 0.5 mL of triflic acid, and anisole (1 mL) was placed in a flask with stir-bar, under N₂. After stirring overnight the liquids were removed under reduced pressure, and the solid was triturated with a saturated sodium bicarbonate solution (15 mL), filtered, washed with water, partially dried, and triturated with CH₃OH (5 mL) for 1 h. After filtration and drying, the desired product was obtained. ¹H NMR (DMSO-d₆: δ 6.52 (br s, 2H), 7.45 (br s, 1H), 7.62-7.80 (m, 5H), 7.83 (br s, 1H), 7.90 (d, 2H, J=8.8 Hz), 8.02 (d, 2H, J=8.8 Hz), 8.63 (s, 1H), 8.69 (s, 1H). ESI mass spectrum for CH₁₈H₁₆N₆O₃: 383 (M+1).

### Example 184

### methyl 4-{3-(aminocarbonyl)-4-[(aminocarbonyl)amino]-1H-pyrazol-1-yl}benzoate

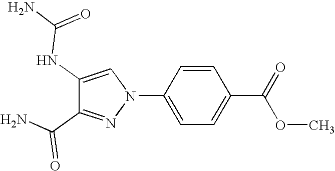

### Step 1: Preparation of methyl 4-[4-amino-3-(aminocarbonyl)-1H-pyrazol-1-yl]benzoate

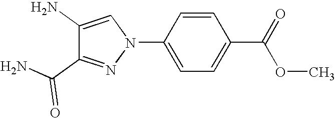

4-Amino-1-(4-bromophenyl)-1H-pyrazole-3-carboxamide (5 g, 17.8 mmol, prepared according to the process of Example 214, Steps 1-3), DPPF (0.382 g, 0.6 mmol), DMF (24 mL), and CH₃OH (20 mL) were combined in a tube containing a stir-bar. N₂ was bubbled through the solution for 30 min to remove oxygen, after that Pd(OAc)₂ (0.166 g, 0.74 mmol) was added, and the reaction mixture was purged again with N₂. The tube was placed in an oil bath at 80° C., and CO was bubbled in for 6 h under vigorous stirring. The reaction mixture was filtered through celite and washed with 10 mL of DMF. Most of the liquids were removed under reduced pressure, and 60 mL of CH₂Cl₂ was added to the residue. The mixture was triturated for 2 h, filtered, and dried. The solid was triturated with water (60 mL) for 5 h, filtered, dried, and triturated with 25 mL of ethanol overnight, to give after filtration and drying the desired product. ¹H NMR (CD₃OD): δ 3.95 (s, 3H), 7.84 (s, 1H), 7.91 (d, 2H, J=8.8 Hz), 8.12 (d, 2H, J=8.8 Hz). ESI mass spectrum for C₁₂H₁₃N₄O₃⁺: 261 (M+1).

### Step 2: Preparation of methyl 4-{3-(aminocarbonyl)-4-[(aminocarbonyl)amino]-1H-pyrazol-1-yl}benzoate

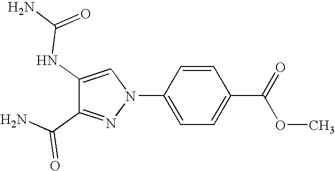

Methyl 4-[4-amino-3-(aminocarbonyl)-1H-pyrazol-1-yl]benzoate (Step 1, 0.465 g, 1.78 mmol) and sodium cyanate (0.405 g) were partially dissolved in a mixture of acetic acid (7 mL) and water (2 mL). The suspension was stirred overnight at rt. Most of solvents were removed after reduced pressure and water (25 mL) was added to the residue. After trituration for 3 h, the solid was filtered, washed with 2N sodium bicarbonate solution, and dried. The dry solid was dissolved in a minimal amount of DMSO. The obtained solution was added with stirring to 100 mL of CH₂Cl₂ and after 1 h was filtered and dried to give the desired product. ¹H NMR (DMSO-d₆): δ 3.85 (s, 3H 6.57 (br s, 2H), 7.58 (br s, 1H), 7.85 (br s, 1H), 8.07 (s, 4H), 8.65 (s, 1H), 8.7 (s, 1H). ESI mass spectrum for C₁₃H₁₄N₅O₄⁺: 304 (M+1).

### Example 185

### methyl 3-{3-(aminocarbonyl)-4-[(aminocarbonyl)amino]-1H-pyrazol-1-yl}benzoate

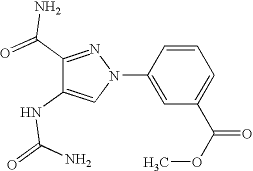

Prepared according to the same procedure as Example 184. ¹H NMR (300 MHz, DMSO-d₆): δ 3.90 (s, 3H) 6.54 (m, 2H) 7.55 (m, 1H) 7.65 (t, J=7.85 Hz, 1H) 7.89 (m, 2H) 8.14 (m, 1H) 8.37 (m, 1H) 8.62 (s, 1H) 8.70 (s, 1H). ESI mass spectrum for C₁₃H₁₄N₅O₄⁺: 304.1 (M+1).

### Example 186

### 4-[(aminocarbonyl)amino]-1-[3′-(2-cyanoethyl)-1,1′-biphenyl-4-yl]-1H-pyrazole-3-carboxamide

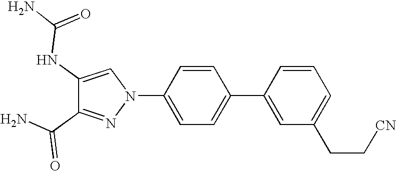

4-[(aminocarbonyl)amino]-1-{3′-[(E)-2-cyanovinyl]-1,1′-biphenyl-4-yl}-1H-pyrazole-3-carboxamide (Example 32, 0.126 g, 0.3 mmol) and sodium borohydride (0.015 g, 0.4 mmol) were partially dissolved in the mixture of pyridine (2 mL) and CH₃OH (4 mL). The suspension was stirred 24 h under reflux. Most of solvents were removed after reduced pressure and water (25 mL) was added to the residue. After trituration for 3 h, the solid was filtered, and dried. HPLC/MS showed the presence of the starting material and the desired product. A preparative reverse phase chromatography was used to isolate the desired product. ¹H NMR (DMSO-d₆): δ 2.85-3.03 (m, 4H), 6.53 (br s, 2H), 7.32 (d, 1H, J=7.6 Hz), 7.45 (t, 1H, J=7.6 Hz), 7.55 (br s, 1H), 7.62 (d, 1H, J=7.6 Hz), 7.69 (s, 1H), 7.80-7.87 (m, 3H), 8.00 (d, 2H, J=8.8 Hz), 8.62 (s, 1H), 8.71 (s, 1H). ESI mass spectrum for C₂₀H₁₁N₆O₂⁺: 375 (M+1).

### Example 187

### 4-[(aminocarbonyl)amino]-1-[4′-(1H-tetrazol-5-yl)-1,1′-biphenyl-4-yl]-1H-pyrazole-3-carboxamide

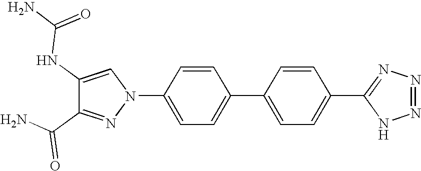

To a solution of 4-[(aminocarbonyl)amino]-1-(4′-cyano-1,1′-biphenyl-4-yl)-1H-pyrazole-3-carboxamide (Example 100, 0.29 g, 0.84 mmol) in DMF (3 mL), sodium azide (0.06 g, 0.92 mmol), and NH₄Cl (0.05 g, 0.92 mmol) were added and the mixture was heated to 100° C. for 24 h. The solvent was removed under reduced pressure, and the residue was triturated with 25 mL of water for 3 h, filtered, partially dried and triturated with CH₃OH (10 mL) for 3 h. After filtration and drying, the desired product was obtained. ¹H NMR (DMSO-d₆: 6.55 (br s, 2H), 7.56 (br s, 1H), 7.67-8.20 (m, 10H), 8.62 (s, 1H), 8.72 (s, 1H). ESI mass spectrum for C₁₈H₁₆N₉O₂⁺: 390 (M+1).

### Example 188

### 4-[(aminocarbonyl)amino]-1-(3,4-dichlorophenyl)-1H-pyrazole-3-carboxamide

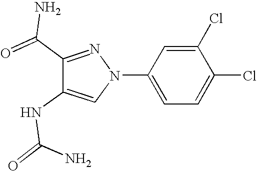

Prepared according to Example 214, step 4. ¹H NMR (300 MHz, DMSO-d₆): δ 6.53 (s, 2H) 7.55 (s, 1H) 7.73 (d, J=8.86 Hz, 1H) 7.90 (d, J=2.62 Hz, 1H) 7.93 (d, J=2.62 Hz, 1H) 8.28 (d, J=2.42 Hz, 1H) 8.65 (s, 1H) 8.67 (s, 1H). ESI mass spectrum for C₁₁H₁₀C₁₂N₅O₂⁺: 314.0 (M+1).

### Example 189

### 1-(3-carbamoyl-1-(4-(cyclohexylthio)phenyl)-1H-pyrazol-4-yl)urea

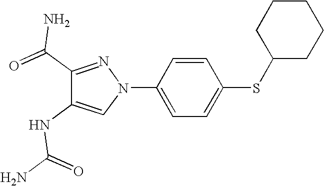

A mixture (0.106 g) containing 67 mole % 4-[(aminocarbonyl)amino]-1-(4-iodophenyl)-1H-pyrazole-3-carboxamide (0.200 mmol, prepared according to the process of Example 214) and 33 mole % 4-[(aminocarbonyl)amino]-1-(4-bromophenyl)-1H-pyrazole-3-carboxamide (Example 19, 0.098 mmol) was combined with copper(1) iodide (0.061 g, 0.32 mmol), cesium acetate (0.168 g, 0.875 mmol), and anhydrous DMF (0.40 mL). The reaction flask was partially evacuated and backfilled with N₂ three times, then cyclohexyl mercaptan (0.038 mL, 0.31 mmol) was added and the flask placed in an oil bath at 90° C. Upon heating the excess pressure was vented off. After 16 h, 1.0 mL anhydrous DMF was added and the flask heated at 100° C. for 24.5 h. The DMF was then stripped, the contents sonicated in 25 mL water for 30 min., filtered, and then the product washed with 25 mL water. The product was then mostly dissolved in 100 mL of 20:80 CH₃OH:EtOAc and filtered through a silica plug. The solvents were then stripped and the product further purified by reverse phase preparative HPLC. The fractions containing product were then partially stripped of solvents, the resulting precipitate was filtered, and the product was washed with 25 mL water. The purified product was dried under vacuum to give a white solid. ¹H NMR (CD₃OD): δ 1.24-1.44 (m, 5H), 1.58-1.66 (m,1H), 1.72-1.83 (m, 2H), 1.93-2.04 (m, 2H),3.15-3.24 (m, 1H), 7.49 (dm, 2H, J=8.9 Hz), 7.75 (dm, 2H, J=8.9 Hz), 8.48 (s, 1H). ESI mass spectrum: 360 (M+1).

### Example 190

### 1-(1-(4-(2-fluorophenylthio)phenyl)-3-carbamoyl-1H-pyrazol-4-yl)urea

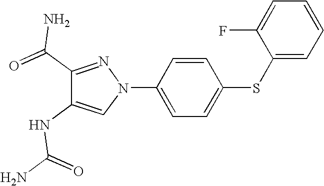

A mixture (0.499 g) containing 67 mole % 4-[(aminocarbonyl)amino]-1-(4-iodophenyl)-1H-pyrazole-3-carboxamide (prepared according to the process of Example 214) and 33 mole % 4-[(aminocarbonyl)amino]-1-(4-bromophenyl)-1H-pyrazole-3-carboxamide (Example 19, 0.463 mmol) was combined with copper(1) iodide (0.274 g, 1.44 mmol), cesium acetate (0.536 g, 2.79 mmol), and anhydrous DMF (7 mL). The reaction flask was partially evacuated and backfilled with N₂ three times, then 2-fluorobenzenethiol (0.16 mL, 1.5 mmol) was added and the flask placed in an oil bath at 100° C. Upon heating the excess pressure was vented off. After 37 h at 100° C. the solvent was stripped and the residue was sonicated in 100 mL water for 30 min., filtered, and washed with 50 mL water. The product was then purified by reverse phase preparative HPLC and then dried under vacuum to give a white solid. ¹H NMR (CD₃OD): δ 7.14-7.21 (m, 2H), 7.33-7.43 (m, 4H), 7.79 (dm, 2H, J=8.9 Hz), 8.49 (s, 1H). ESI mass spectrum: 372 (M+1).

### Example 191

### 1-(3-carbamoyl-1-(4-(cyclohexylsulfinyl)phenyl)-1H-pyrazol-4-yl)urea

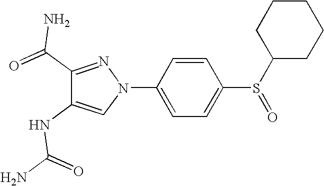

1-(3-carbamoyl-1-(4-(cyclohexylthio)phenyl)-1H-pyrazol-4-yl)urea (Example 189, 0.034 g, 0.095 mmol), glacial acetic acid (0.30 mL), and 30% hydrogen peroxide (0.011 mL, 0.11 mmol) were combined in a vial and the mixture stirred overnight at rt. Water was added, and the resulting precipitate was filtered and washed several times with 1-2 mL water. The product was air dried to give the title compound as a white solid. ¹H NMR (DMSO-d₆): δ 1.03-1.36 (m, 5H), 1.44-1.52 (m, 1H), 1.52-1.60 (m, 1H), 1.67-1.79 (m, 2H), 1.82-1.90 (m, 1H), 2.68-2.77 (m, 1H), 6.51 (br s, 2H), 7.54 (br s, 1H), 7.69 (dm, 2H, J=8.9 Hz), 7.82 (br s, 1H), 8.08 (dm, 2H, J=8.9 Hz), 8.63 (s, 1H), 8.67 (s, 1H). ESI mass spectrum: 376 (M+1).

### Example 192

### 1-(3-carbamoyl-1-(4-(cyclohexylsulfonyl)phenyl)-1H-pyrazol-4-yl)urea

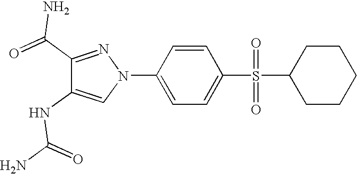

1-(3-carbamoyl-1-(4-(cyclohexylthio)phenyl)-1H-pyrazol-4-yl)urea (Example 189, 0.038 g, 0.11 mmol), glacial acetic acid (0.30 mL), and 30% hydrogen peroxide (0.030 mL, 0.29 mmol), were combined in a vial and the vial was placed in an oil bath at 100° C. for 60 min. The acetic acid was stripped, then the residue triturated in 5 mL water for 30 min, followed by trituration in 1.4 mL anhydrous diethyl ether overnight, filtering, and washing with 2 mL anhydrous diethyl ether. The solid was then dried under vacuum to give a tan solid. ¹H NMR (DMSO-d₆): δ 1.00-1.32 (m, 6H), 1.52-1.61 (m, 1H), 1.68-1.77 (m, 2H), 1.83-1.92 (m, 2H), 6.54 (br s, 2H), 7.60 (br s, 1H), 7.90 (dm+br s, 3H, J=9.0 Hz), 8.16 (dm, 2H, J=8.9 Hz), 8.69 (s, 2H). ESI mass spectrum: 392 (M+1).

### Example 193

### 1-(1-(4-(2-fluorophenylsulfinyl)phenyl)-3-carbamoyl-1H-pyrazol-4-yl)urea

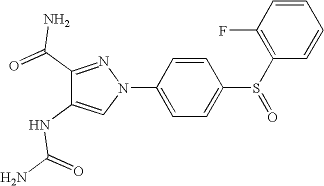

1-(1-(4-(2-fluorophenylthio)phenyl)-3-carbamoyl-1H-pyrazol-4-yl)urea (Example 190, 0.050 g, 0.13 mmol), glacial acetic acid (0.4 mL), and 30% hydrogen peroxide (0.0145 mL, 0.14 mmol) were combined in a vial and the mixture stirred overnight at rt. 5 mL water was added and the resulting precipitate was filtered and washed with 5 mL water. Then glacial acetic acid (0.5 mL), and 30% hydrogen peroxide (0.009 mL, 0.009 mmol) were added to the product and the mixture was stirred for 20 h at rt. Again 5 mL water was added, and the resulting precipitate was filtered and washed twice with 5 mL water. Then glacial acetic acid (0.4 mL), and 30% hydrogen peroxide (0.011 mL, 0.011 mmol) were added to the product and the mixture was stirred overnight at rt. 5 mL water was added and the resulting precipitate was filtered and washed twice with 5 mL water. The product was dried under vacuum to give a white solid. ¹H NMR (DMSO-d₆): δ 6.50 (br s, 2H), 7.29-7.35 (m, 1H), 7.47 (td, 1H, J=7.6, 0.94 Hz), 7.53-7.62 (m, 2H), 7.75-7.86 (m, 4H), 8.06 (dm, 2H, J=8.9 Hz), 8.54 (s, 1H), 8.60 (s, 1H). ESI mass spectrum: 388 (M+1).

### Example 194

### 1-(1-(4-(2-fluorophenylsulfonyl)phenyl)-3-carbamoyl-1H-pyrazol-4-yl)urea

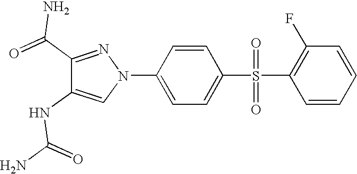

1-(1-(4-(2-fluorophenylthio)phenyl)-3-carbamoyl-1H-pyrazol-4-yl)urea (Example 190, (0.049 g, 0.13 mmol), glacial acetic acid (0.6 mL), and 30% hydrogen peroxide (0.040 mL, 0.39 mmol) were combined in a vial and the vial was placed in an oil bath at 100° C. for 1.2 h. The solvent was then mostly stripped, 5 mL water was added, the resulting precipitate was filtered, and washed with 5 mL water. Then glacial acetic acid (0.50 mL) and 30% hydrogen peroxide (0.023 mL, 0.22 mmol) was added to the product and the mixture was placed in an oil bath at 100° C. for 1.0 h. Water (5 mL) was then added, the resulting precipitate filtered, and washed twice with 5 mL water. The product was then triturated overnight with roughly 2 mL anhydrous diethyl ether, filtered, and washed with 2 mL anhydrous diethyl ether. The product was dried under vacuum to give a tan solid. ¹H NMR (CD₃OD): δ 7.22-7.29 (m, 1H), 7.41-7.46 (m, 1H), 7.67-7.75 (m, 1H), 8.05-8.15 (m, 5H), 8.62 (s, 1H). ESI mass spectrum: 404 (M+1).

### Example 195

### 1-(3-carbamoyl-1-(3-iodophenyl)-1H-pyrazol-4-yl)urea

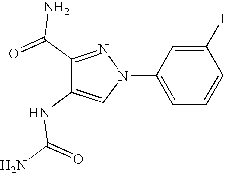

4-[(aminocarbonyl)amino]-1-(3-bromophenyl)-1H-pyrazole-3-carboxamide (Example 20, 1.736 g, 5.356 mmol), copper(1) iodide (1.008 g, 5.29 mmol), sodium iodide (1.611 g, 10.75 mmol), N,N′-dimethylethylenediamine (1.1 mL, 10.3 mmol), and anhydrous 1,4-dioxane (11 mL) were combined in a Fisher Porter bottle. The bottle was flushed with N₂, sealed, and then placed in an oil bath at 110° C. After 42 h the bottle was removed from the oil bath. The contents were stripped of solvent, 200 mL water was added, then sonicated for 30 min, filtered, and washed twice with 50 mL portions of water. The product was then purified by reverse phase preparative HPLC. The purified product was dried under vacuum to give a white solid. ¹H NMR (CD₃OD): δ 7.25 (t, 1H, J=8.1 Hz), 7.69 (dm, 1H J=7.8 Hz), 7.80 (dm, 1H, J=8.2 Hz), 8.24 (t, 1H, J=1.9 Hz), 8.50 (s, 1H). ESI mass spectrum: 372 (M+1).

### Example 196

### 1-(1-(4-(4-fluorophenylthio)phenyl)-3-carbamoyl-1H-pyrazol-4-yl)urea

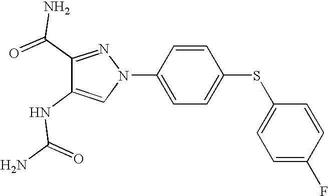

A mixture (0.502 g) containing 67 mole % 4-[(aminocarbonyl)amino]-1-(4-iodophenyl)-1H-pyrazole-3-carboxamide (0.946 mmol, prepared according to the process of Example 214) and 33 mole % 1-(1-(4-bromophenyl)-3-carbamoyl-1H-pyrazol-4-yl)urea (Example 19, 0.466 mmol) was combined with copper(1) iodide (0.285 g, 1.5 mmol), cesium acetate (0.872 g, 4.54 mmol), and anhydrous DMF (7 mL). The reaction flask was partially evacuated and backfilled with N₂ three times, then 4-fluorobenzenethiol (0.175 mL, 1.63 mmol) was added and the flask placed in an oil bath at 100° C. Upon heating the excess pressure was vented off. After 22.5 h at 100° C. the solvent was stripped and the residue sonicated in 100 mL water for 30 min., filtered, and washed with 50 mL water. After air drying, sodium cyanate (0.145 g, 2.23 mmol), water (2.4 mL), and glacial acetic acid (8.5 mL) were added and stirred overnight. Then 3.0 mL water was added, the slurry filtered, and washed twice with 5 mL water. After air drying, the product was sonicated in 25 mL ethanol, filtered, and the solid washed with 5 mL ethanol. The ethanol was then stripped down to roughly 10 mL and 100 mL water was added. The resulting precipitate was then filtered, washed with 50 mL water, and allowed to air dry 3 days. The title compound was a yellow solid. ¹H NMR (CD₃OD): δ 7.13 (t m, 2H, J=1.9 Hz), 7.36 (dm, 2H, J=8.9 Hz), 7.42-7.48 (m, 2H), 7.77 (dm, 2H, J=8.9 Hz), 8.48 (s, 1H). ESI mass spectrum: 372 (M+1).

### Example 197

### 1-(1-(4-(3-fluorophenylthio)phenyl)-3-carbamoyl-1H-pyrazol-4-yl)urea

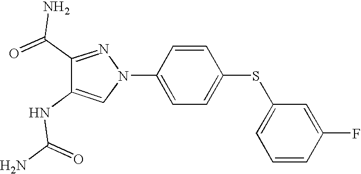

A mixture (0.407 g) containing 67 mole % 4-[(aminocarbonyl)amino]-1-(4-iodophenyl)-1H-pyrazole-3-carboxamide (0.767 mmol, prepared according to the process of Example 214) and 33 mole % 1-(1-(4-bromophenyl)-3-carbamoyl-1H-pyrazol-4-yl)urea (Example 19, 0.378 mmol) was combined with copper(1) iodide (0.227 g, 1.19 mmol), cesium acetate (0.537 g, 2.80 mmol), and anhydrous DMF (6 mL). The reaction flask was partially evacuated and backfilled with N₂ three times, then 3-fluorobenzenethiol (0.110 mL, 1.30 mmol) was added and the flask placed in an oil bath at 100° C. Upon heating the excess pressure was vented off. After 40 h at 100° C., the DMF was stripped and the residue sonicated in 50 mL water for 30 min., filtered, and washed with 25 mL water. The product was partially purified by reverse phase preparative HPLC. The fractions containing product were combined and partially stripped of solvent. Then saturated sodium bicarbonate was added, the mixture filtered, and the product washed with water. The product was the sonicated for 30 min. in 2.0 mL ethanol and filtered. After air drying, sodium cyanate (0.044 g, 0.68 mmol), water (1.3 mL), and glacial acetic acid (4.5 mL) were added and stirred overnight. Then 1.5 mL water was added, the slurry filtered, and the solid washed twice with 5 mL water. The product precipitated in the filtrate when the water washes were added and the resulting mixture was filtered, washed with 5 mL water, and then dried under vacuum. The title compound was a tan solid. ¹H NMR (CD₃OD): δ 6.95-7.03 (m, 2H), 7.11 (dm, 1H, J=7.9 Hz), 7.30-7.37 (m, 1H), 7.52 (dm, 2H, J=8.9 Hz), 7.85 (dm, 2H, J=8.9 Hz), 8.53 (s, 1H). ESI mass spectrum: 372 (M+1)

### Example 198

### 1-(1-(3-(2-fluorophenylthio)phenyl)-3-carbamoyl-1H-pyrazol-4-yl)urea

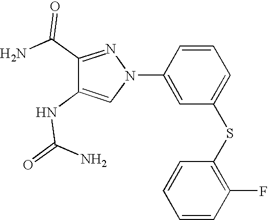

1-(3-carbamoyl-1-(3-iodophenyl)-1H-pyrazol-4-yl)urea (Example 195, 0.100 g, 0.269 mmol), copper(1) iodide (0.062 g, 0.33 mmol), cesium acetate (0.162 g, 0.844 mmol), and anhydrous DMF (1.4 mL) were combined. The reaction flask was partially evacuated and backfilled with N₂ three times, then 2-fluorobenzenethiol (0.040 mL, 0.37 mmol) was added and the flask placed in an oil bath at 100° C. Upon heating the excess pressure was vented off. After 24 h at 100° C., the DMF was stripped and the residue was sonicated in 25 mL water for 30 min., filtered, and washed with 25 mL water. After air drying, sodium cyanate (0.045 g, 0.69 mmol), water (1.3 mL), and glacial acetic acid (4.5 mL) were added and stirred overnight. Then 1.5 mL water was added, the slurry filtered, and the solid washed twice with 5 mL water. Product precipitated in the filtrate when the water washes were added and the resulting mixture was filtered, washed with 5 mL water, and then dried under vacuum. The title compound was a light pink solid. ¹H NMR (CD₃OD): δ 7.15-7.23 (m, 3H), 7.37-7.47 (m, 3H), 7.69 (ddd, 1H, J=8.2, 2.3, 0.88 Hz), 7.79 (t, 1H, J=1.9 Hz), 8.45 (s, 1H). ESI mass spectrum: 372 (M+1)

### Example 199

### 1-(1-(4-(4-hydroxyphenylthio)phenyl)-3-carbamoyl-1H-pyrazol-4-yl)urea

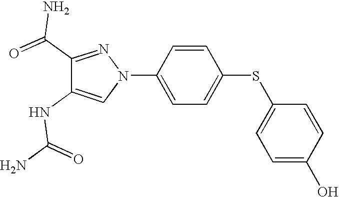

A mixture (0.500 g) containing 67 mole % 4-[(aminocarbonyl)amino]-1-(4-iodophenyl)-1H-pyrazole-3-carboxamide (0.942 mmol, prepared according to the process of Example 214) and 33 mole % 1-(1-(4-bromophenyl)-3-carbamoyl-1H-pyrazol-4-yl)urea (Example 19, 0.464 mmol) was combined with 4-mercaptophenol (0.230 g, 1.82 mmol), copper(1) iodide (0.287 g, 1.51 mmol), cesium acetate (0.675 g, 3.52 mmol), and anhydrous DMF (7 mL). The reaction flask was partially evacuated and backfilled with N₂ three times, then placed in an oil bath at 100° C. Upon heating the excess pressure was vented off. After 22.5 h at 100° C. the solvent was stripped and the residue sonicated in 100 mL water for 30 min., filtered, and washed with 50 mL water. The product was then triturated in 25 mL CHCl₃ for 1.5 h, sonicated for 30 min., filtered, and washed with 25 mL CHCl₃. The product was then sonicated for 30 min. in 25 mL ethanol, filtered, and the solid washed with 5-6 mL ethanol. The ethanol filtrate was stripped to 10 mL, 100 mL water was added, the resulting slurry filtered, and the product washed with 25 mL water. The product was sonicated for 5 min. in 150 mL 1 N NaOH solution, filtered, and the solid washed with 25 mL water. The filtrate was neutralized with 150 mL 1 N HCl solution. The resulting precipitate was filtered and washed with 25 mL water. After air drying, sodium cyanate (0.265 g, 4.08 mmol), water (1.3 mL), and glacial acetic acid (4.5 mL) were added and stirred overnight. Then 1.5 mL water was added, the slurry filtered, and the solid washed twice with 5 mL water. Product precipitated in the filtrate when the water washes were added, and the mixture was then filtered and washed with 5 mL water. The product was further purified by reverse phase preparative HPLC, and dried under vacuum to give a white solid. ¹H NMR (CD₃OD): δ 6.83 (dm, 2H, J=8.7 Hz), 7.19 (dm, 2H, J=8.9 Hz), 7.35 (dm, 2H, J=8.7 Hz), 7.68 (dm, 2H, J=8.9 Hz), 8.43 (s, 1H). ESI mass spectrum: 370 (M+1).

### Example 200

### 1-(1-(4-(4-fluorophenylsulfinyl)phenyl)-3-carbamoyl-1H-pyrazol-4-yl)urea

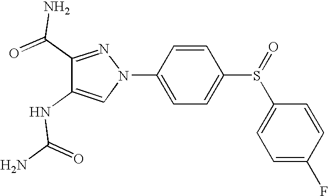

1-(1-(4-(4-fluorophenylthio)phenyl)-3-carbamoyl-1H-pyrazol-4-yl)urea (Example 196, 0.080 g, 0.19 mmol), glacial acetic acid (0.80 mL), and 30% hydrogen peroxide (0.026 mL, 0.25 mmol), were combined in a vial and the mixture stirred for 89 h at rt. The solvent was then stripped off, and the product purified by reverse phase preparative HPLC. The purified product was dried under vacuum to give a yellow solid. ¹H NMR (CD₃OD/DMSO-d₆(1:4)): δ 7.31 (t m, 2H, J=8.9), 7.72-7.79 (m, 4H), 7.99 (dm, 2H, J=8.9 Hz), 8.57 (s, 1H). ESI mass spectrum: 388 (M+1).

### Example 201

### 1-(1-(4-(3-fluorophenylsulfinyl)phenyl)-3-carbamoyl-1H-pyrazol-4-yl)urea

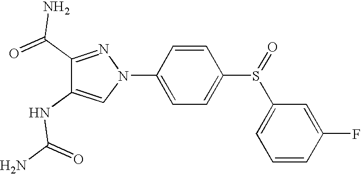

Prepared according to Example 200 (starting with Example 197).

The title compound was a white solid. ¹H NMR (CD₃OD/DMSO-d₆(1:4)): δ 7.23-7.29 (m, 1H), 7.49-7.57 (m, 3H), 7.81 (dm, 2H, J=8.9 Hz), 7.99 (dm, 2H, J=8.9 Hz), 8.56 (s, 1H). ESI mass spectrum: 388 (M+1).

### Example 202

### 1-(1-(3-(2-fluorophenylsulfinyl)phenyl)-3-carbamoyl-1H-pyrazol-4-yl)urea

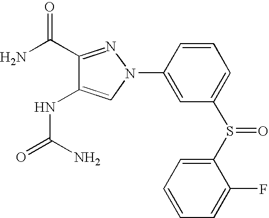

Prepared according to Example 200 (starting with Example 198).

The title compound was a yellow solid. ¹H NMR, (CD₃OD/DMSO-d₆(1:4)): δ 7.21-7.27 (m, 1H), 7.40 (td, 1H, J=7.6, 1.0 Hz), 7.50-7.57 (m, 2H), 7.62 (t, 1H, J=7.9 Hz), 7.80-7.86 (m, 1H), 7.97 (ddd, 1H, J=8.1, 1.1, 0.87 Hz), 8.17-8.20 (m, 1H), 8.57 s, 1H). ESI mass spectrum: 388 (M+1)

### Example 203

### 1-(1-(4-(3-fluorophenylsulfonyl)phenyl)-3-carbamoyl-1H-pyrazol-4-yl)urea

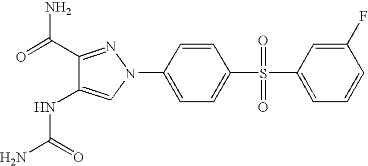

1-(1-(4-(3-fluorophenylthio)phenyl)-3-carbamoyl-1H-pyrazol-4-yl)urea (Example 197, 0.22 g, 0.052 mmol, glacial acetic acid (0.40 mL), and 30% hydrogen peroxide (0.014 mL, 0.14 mmol) were combined in a vial and then the vial was placed in an oil bath at 100C for 68 h. The vial was removed from the oil bath, the solvent stripped, and the product was then purified by reverse phase preparative HPLC. The purified product was dried under vacuum to give a tan solid. ¹H NMR (CD₃OD/DMSO-d₆(1:4)): δ 7.41-7.49 (m, 1H), 7.58-7.65 (m, 1H), 7.74-7.81 (m, 2H), 8.05 (s, 4H), 8.62 (s, 1H). ESI mass spectrum: 404 (M+1).

### Example 204

### 1-(1-(4-(4-fluorophenylsulfonyl)phenyl)-3-carbamoyl-1H-pyrazol-4-yl)urea

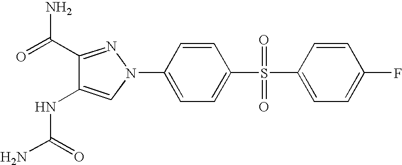

1-(3-carbamoyl-1-(4-iodophenyl)-1H-pyrazol-4-yl)urea (Example 215, 1.004 g, 2.71 mmol), copper (I) iodide (0.051 g, 0.27 mmol), (trans,6E,7E)—N1,N2-bis((pyridin-2-yl)methylene)cyclohexane-1,2-diamine (0.161 g, 0.551 mmol), Cs₂CO₃ (0.878 g, 2.69 mmol), 4A molecular sieves (0.809 g), and anhydrous DMF (4.0 mL) were combined. The reaction flask was partially evacuated and backfilled with N₂ several times, then 4-fluorobenzenethiol (0.43 mL, 4.0 mmol) was added and the flask placed in an oil bath at 110° C. Upon heating the excess pressure was vented off. After 4.0 h at 110° C., 40 mL DMF was added, the mixture was filtered at rt, and the solid was washed with 10 mL of DMF. The DMF filtrate was stripped of solvent, the residue was sonicated for 30 min. in 75 mL of water, filtered, and the solid was washed with 25 mL of water. The solid was then dissolved in 250 mL THF, filtered, the filtrate was stripped of solvent, and the residue dried under vacuum. The residue was then combined with 11.5 mL glacial acetic acid, and 30% hydrogen peroxide (0.80 mL, 7.8 mmol). The reaction vessel was then place in a 100C oil bath. After 1 h, hydrogen peroxide (0.55 mL, 5.4 mmol) was added. After another 1.1 h, the vessel was removed from the oil bath, 90 mL water was added, and the resulting precipitate filtered. The product was then partially purified by reverse phase preparative HPLC. The purer fractions were combined, partially stripped of solvent, and filtered. The less pure fractions were combined, stripped of solvent, and were recrystalized from acetonitrile and water several times. The purified product was dried under vacuum to give a yellow solid. ¹H NMR (DMSO-d₆): δ 6.53 (br s, 2H), 7.42-7.49 (m, 2H), 7.59 (brs, 1H), 7.88 (brs, 1H), 8.03-8.15 (m, 6H), 8.65 (s, 1H), 8.67 (s, 1H). ESI mass spectrum: 404 (M+1)

### Example 205

### 1-(3-carbamoyl-1-(4-(pyridin-2-ylthio)phenyl)-1H-pyrazol-4-yl)urea

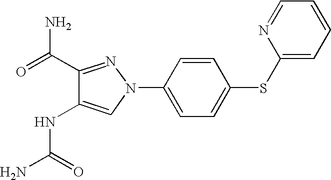

4-[(aminocarbonyl)amino]-1-(4-iodophenyl)-1H-pyrazole-3-carboxamide (0.595 g, 1.60 mmol, prepared according to the process of Example 214), 2-mercaptopyridine (0.272 g, 2.45 mmol), copper (I) iodide (0.033 g, 0.17 mmol), (trans,6E,7E)-N1,N2-bis((pyridin-2-yl)methylene)cyclohexane-1,2-diamine (0.095 g, 0.32 mmol), Cs₂CO₃ (0.689 g, 2.11 mmol), 4A molecular sieves (0.495 g), and anhydrous DMF (2.0 mL) were combined. The reaction flask was partially evacuated and backfilled with N₂ three times, and the flask placed in an oil bath at 110° C. Upon heating the excess pressure was vented off. After 23 h at 110° C., 70 mL DMF was added after cooling to rt, the mixture filtered, and the filtrate stripped of solvent. The residue was then sonicated in 75 mL water for 30 min., filtered, and washed with 25 mL water. The product was then partially purified by reverse phase preparative HPLC. The product was then mostly dissolved in 25 mL 1 N HCl solution and filtered. The filtrate was then neutralized with saturated sodium bicarbonate, the resulting precipitate filtered, and the product washed with 25 mL water. Then 20 mL of 0.2N HCl solution was added, followed by more HCl until roughly 25 mL 0.6N HCl was added. The slurry was then filtered, 25-30 mL 1 N HCl was added to the HCl salt, and the slurry filtered. Then 100 mL water was added, and the product salt partially dissolved. The resulting mixture was neutralized with saturated sodium bicarbonate, the slurry was then filtered, washed with 25 mL water, and air dried overnight to give a white solid. ¹H NMR (DMSO-d₆): δ 6.51 (br s, 2H), 7.00 (d, 1H, J=8.1 Hz), 7.12-7.17 (m, 1H), 7.53 (brs, 1H), 7.61-7.70 (m, 3H), 7.81 (brs, 1H), 7.99 (dm, 2H, J=8.6 Hz), 8.39 (dm, 1H, J=3.9 Hz), 8.61 (s, 1H), 8.67 (s, 1H). ESI mass spectrum: 355 (M+1)

### Example 206

### 1-(3-carbamoyl-1-(4-(pyridin-4-ylthio)phenyl)-1H-pyrazol-4-yl)urea

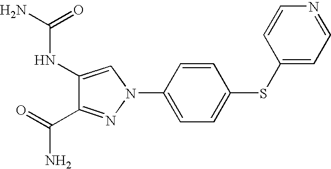

Made similarly to Example 205, except 4-mercaptopyridine was used, and when the 0.2N HCl was added, the product dissolved. The HCl solution was then partially neutralized with saturated sodium bicarbonate and the resulting precipitate filtered. The filtrate was then neutralized with more saturated sodium bicarbonate and the resulting precipitate was filtered, washed with water, and purified by reverse phase preparative HPLC. The product was dried under vacuum.

The title compound was a yellow solid. ¹H NMR (DMSO-d₆): δ 6.52 (br s, 2H), 7.04 (dd, 2H, J=4.7, 1.5 Hz), 7.55 (br s, 1H), 7.69 (dm, 2H, J=8.7 Hz), 7.83 (br s, 1H), 8.04 (dm, 2H, J=8.9 Hz), 8.36 (br d, 2H, J=5.2 Hz), 8.63 (s, 1H), 8.68 (s, 1H). ESI mass spectrum: 355 (M+1).

### Example 207

### 1-(3-carbamoyl-1-(4-(cyclopentylthio)phenyl)-1H-pyrazol-4-yl)urea

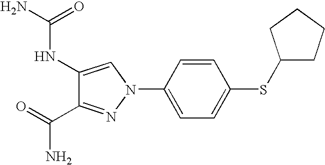

Made similarly to Example 205, except that the cyclopentyl mercaptan was added after the flask was partially evacuated and backfilled with N₂ several times and after 6 h at 110° C., the flask was removed from the oil bath and additional copper (I) iodide (0.030 g, 0.16 mmol), (trans,6E,7E)-N1,N2-bis((pyridin-2-yl)methylene)cyclohexane-1,2-diamine (0.093 g, 0.32 mmol), and anhydrous DMF (2.0 mL) were added. The reaction flask was then partially evacuated and backfilled with N₂ as before, and the flask placed back in the 110° C. oil bath. The excess pressure was again vented upon heating. Then after another 5 h at 110° C., 40 mL DMF was added, the mixture was filtered, and the solid was washed with 10 mL of DMF. The DMF filtrate was stripped of solvent, the residue sonicated for 30 min. in 75 mL of water, filtered, and the solid was washed with 25 mL of water. The solid was then mostly dissolved in 500 mL boiling THF and filtered hot. The THF was then stripped and the residue purified by reverse phase preparative HPLC. The purified product was dried under vacuum. The title compound was a light tan solid. ¹H NMR (DMSO-d₆): δ 1.44-1.63 (m, 4H), 1.63-1.75 (m, 2H), 1.98-2.08 (m, 2H), 3.68-3.76 (m, 1H), 6.49 (br s, 2H), 7.43 (dm, 2H, J=8.7 Hz), 7.49 (br s, 1H), 7.74 (br s, 1H), 7.80 (dm, 2H, J=8.7 Hz), 8.52 (s, 1H), 8.65 (s, 1H). ESI mass spectrum: 346 (M+1).

### Example 208

### 1-(1-(4-(2-hydroxyphenylthio)phenyl)-3-carbamoyl-1H-pyrazol-4-yl)urea

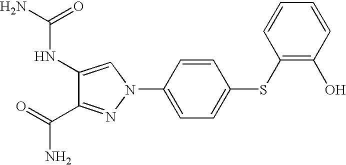

Made similarly to Example 205, except the 2-mercaptophenol was added after the flask was partially evacuated and backfilled with N₂ several times and after 4 h at 110° C., 40 mL DMF was added, the mixture filtered, and the solid washed with 10 mL of DMF. The DMF filtrate was stripped of solvent, the residue was sonicated for 30 min. in 75 mL of water, filtered, and the solid was washed with 25 mL of water. The solid was then mostly dissolved in 500 mL boiling THF and filtered hot. The THF filtrate was then stripped of solvent and the residue purified by reverse phase preparative HPLC. The purified product was dried under vacuum. The title compound was a light tan solid. ¹H NMR (DMSO-d₆): δ 6.48 (br s, 2H), 6.79 (tm, 1H, J=7.5 Hz), 6.92 (dd, 1H, J=8.1, 0.9 Hz), 7.14 (dd, 1H, J=7.7, 1.4 Hz), 7.19 (tm, 1H, J=7.7 Hz),7.27 (dm, 2H, J=8.7 Hz), 7.49 (br s, 1H), 7.72 (br s, 1H), 7.81 (dm, 2H, J=8.9 Hz), 8.51 (s, 1H), 8.65 (s, 1H), 10.01 (br s, 1H). ESI mass spectrum: 370 (M+1).

### Example 209

### 4-[(Aminocarbonyl)amino]-1-(3-chloro-4-methoxyphenyl)-1H-pyrazole-3-carboxamide

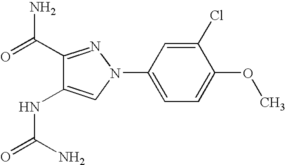

### Step 1: Preparation of ethyl 4-nitro-1H-pyrazole-3-carboxylate

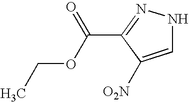

4-Nitro-1H-pyrazole-3-carboxylic acid (2 g, 12.73 mmol; Aldrich) was refluxed in absolute ethanol (25 mL) containing trace sulfuric acid (0.2 mL) for 5 h. The reaction was evaporated and partitioned between EtOAc and 5% NaHCO₃. The EtOAc layer was separated, dried over MgSO₄ and filtered. The EtOAc was removed to give a white solid. ¹H NMR (300 MHz, DMSO-d₆): δ 1.28 (t, J=7 Hz, 3H), 4.34 (q, J=7 Hz, 2H), 8.91 (s, 1H), 14.39 (br s, 1H); MS (ESI+) for C₆H₇N₃O₄ m/z 186.1 (M+H)⁺.

### Step 2: Preparation of N-(2,4-Dimethoxybenzyl)-4-nitro-1H-pyrazole-3-carboxamide

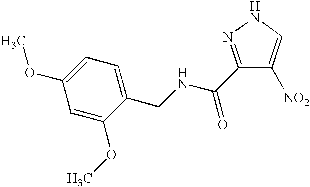

The carboxylate from Step 1 (3.32 g, 17.93 mmol) was stirred in excess 2,4-dimethoxybenzylamine at 120° C. for 1.5 h. The resulting product was triturated with hot EtOH and filtered to give 8.45 g of product as the 2,4-dimethoxybenzylamine salt (1:1). The salt was dissolve in MeOH (300 mL, 60° C. for 0.5 h), acidified using conc. HCl and diluted to a total volume of 1 L with water. The resulting precipitate was filtered, rinsed with water and air-dried to give an off-white solid. ¹H NMR (300 MHz, DMSO-d₆): δ 3.74 (s, 3H), 3.79 (s, 3H), 4.33 (d, 2H), 6.50 (m, 2H), 7.19 (d, 1H), 8.8 (br s, 1H), 8.87 (br s, 1H), 14.05 (br s, 1H); MS (ESI+) for C₁₃H₁₄N₄O₅ m/z 307.0 (M+H)⁺.

### Step 3: Preparation of 4-amino-N-(2,4-dimethoxybenzyl)-1H-pyrazole-3-carboxamide

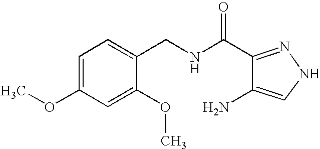

The carboxamide from Step 2 (4.60 g, 15 mmol) was hydrogenated using 10% Pd/C (0.4 g) in MeOH/THF (30 mL/200 mL) at atmospheric pressure (H₂ balloon) for 4 h. The reaction was filtered through Celite and evaporated to give the desired product as a foam. ¹H NMR (300 MHz, DMSO-d₆): δ 3.72 (s, 3H), 3.79 (s, 3H), 4.29 (d, 2H), 4.55 (s, 2H), 6.42 (m, 1H), 6.54 (m, 1H), 7.07 (m, 2H), 7.9 (br s, 1H), 12.53 (s, 1H); MS (ESI+) for C₁₃H₁₆N₄O₃ m/z 277.0 (M+H)⁺.

### Step 4: Preparation of 4-[(aminocarbonyl)amino]-N-(2,4-dimethoxybenzyl)-1H-pyrazole-3-carboxamide

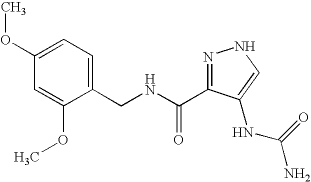

The product of Step 3 (4.07 g, 14.73 mmol) and sodium cyanate (1.91 g, 29.4 mmol) were suspended in water (40 mL). Tetrahydrofuran (40 mL) was added, followed by HOAc (15 mL) and the reaction was stirred at rt for 10 min. The reaction was evaporated and partitioned between EtOAc and 1 N NaOH. The EtOAc layer was separated, dried over MgSO₄, filtered and evaporated to give 5.25 g of product. A portion of this was triturated from EtOAc to give the title compound. ¹H NMR (300 MHz, DMSO-d₆): δ 3.72 (s, 3H), 3.79 (s, 3H), 4.33 (d, 2H), 6.32 (br s, 2H), 6.47 (m, 1H), 6.54 (s, 1H), 7.07 (m, 1H), 7.95 (s, 1H), 8.24 (m, 1H), 8.57 (s, 1H), 12.95 (br s, 1H); MS (ESI+) for C₁₄H₁₇N₅O₄ m/z 320.0 (M+H)⁺.

### Step 5: Preparation of 4-[(Aminocarbonyl)amino]-1-(3-chloro-4-methoxyphenyl)-N-(2,4-dimethoxybenzyl)-1H-pyrazole-3-carboxamide

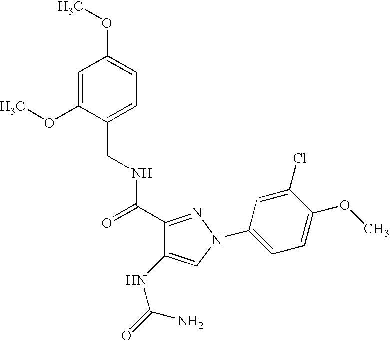

The product of Step 4 (0.250 g, 0.783 mmol), 3-chloro-4-methoxyphenylboronic acid (0.146 g, 0.783 mmol), copper(II) acetate (0.014 g, 0.078 mmol) and pyridine (0.093 g, 1.17 mmol) were combined in MeOH (3 mL) and heated overnight at 70° C. The reaction was evaporated, partitioned between EtOAc and 1 N HCl and the EtOAc layer was removed to give a brown oil. The product was purified on silica gel, eluting with 5% MeOH/CH₂Cl₂ to give the product as a foam. ¹H NMR (300 MHz, CDCl₃): δ 3.79 (s, 3H), 3.86 (s, 3H), 3.92 (s, 3H), 4.53 (d, 2H), 5.06 (m, 2H), 6.46 (m, 2H), 6.94 (m, 1H), 7.21 (m, 1H), 7.34 (m, 1H), 7.48 (m, 1H), 7.78 (m, 1H), 8.43 (s, 1H), 8.93 (s, 1H); MS (ESI+) for C₂₁H₂₂ClN₅O₅ m/z 460.0 (M+H)⁺.

### Step 6: Preparation of 4-[(aminocarbonyl)amino]-1-(3-chloro-4-methoxyphenyl)-1H-pyrazole-3-carboxamide

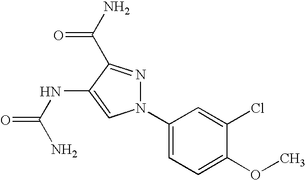

The product of Step 5 (0.022 g, 0.048 mmol) was stirred in neat trifluoroacetic acid for 10 min. and evaporated to give the title compound. ¹H NMR (300 MHz, DMSO-d₆): δ 3.89 (s, 3H), 6.5 (br s, 2H), 7.22 (m, 1H), 7.43 (s, 1H), 7.82 (m, 2H), 8.03 (m, 1H), 8.52 (s, 1H), 8.65 (s, 1H); MS (ESI+) for C₁₂H₁₂ClN₅O₃ m/z 310.0 (M+H)⁺.

### Example 210

### 1-(1-(4-(2-fluorophenylthio)-2-fluorophenyl)-3-carbamoyl-1H-pyrazol-4-yl)urea

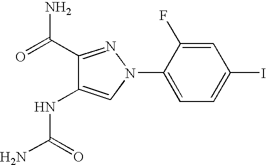

### Step 1: Preparation of (2Z)-2-cyano-2-[(2-fluoro-4-iodophenyl)hydrazono]acetamide

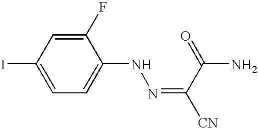

A suspension of 2-fluoro-4-iodoaniline (24.7 g, 104.2 mM) in concentrated HCl (25 mL) and R.O. water (75 mL) was cooled in an ice salt water bath to −10° C. To this suspension was added a solution of sodium nitrite (7.28 g, 105.5 mM) in 20 mL distilled water over 5 min. The mixture was stirred for 25 min., keeping the reaction mixture cold (0-5° C.). An ice-cold solution of sodium acetate trihydrate (41.13 g, 302 mM) in 100 mL water was added to the diazonium salt suspension. This mixture was stirred at −10° C. for 10 min, then added through an ice-jacketed dropping funnel to a −4° C. mixture of 2-cyanoacetamide (25 g, 313 mM), 95% ethanol (95 mL), R.O. water (140 mL)+ice cold sodium acetate (13.865 g, 102 mM) in 30 mL water. During the addition and reaction, the reaction was vigorously stirred with a mechanical stirrer. The addition took 15 min. The orange suspension was stirred an additional 90 min., then the solid product was filtered out, washing with R.O. water (almost 2 gal.) until little yellow color was left in the washings. A sample of the solid product was chromatographed via HPLC, confirming the presence of only the desired product. The product was dried open to the air in a dark hood for three days to give an orange solid. ¹H NMR (300 MHz, DMSO-d₆): δ 7.59 (m, 2H); 7.75 (m, 2H); 7.90 (bs, 1H); 11.11 (s, 1H). LCMS (ESI+) for C₉H₆F₁I₁N₄O₁: m/z 332.9 (M+H)⁺.

### Step 2: Preparation of 4-amino-1-(2-fluoro-4-iodophenyl)-5-propionyl-1H-pyrazole-3-carboxamide

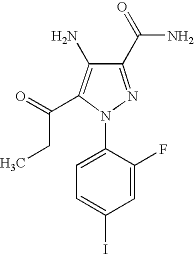

A pressure bottle was charged with the hydrazone from Step 1 (21.2 g, 64 mM), DMF (55 mL), ground potassium carbonate (18.6 g), and ethyl bromoacetate (7.6 mL, 68.5 mM). The solution was purged with N₂, the cap was screwed onto the bottle, and the reaction was heated to 110° C. for 1 h. The reaction was cooled and poured onto ice. The dark mass at the bottom of the flask was poured on ice separately. Both suspensions were filtered and dried in the vacuum oven to give the desired product. A sample of the product was purified by extraction into ethyl acetate followed by crystallization from ethyl acetate-heptane. R_{f}=0.58 in 5% CH₃OH—CHCl₃. ¹H NMR (300 MHz, DMSO-d₆): δ 1.11 (t, 3H); 4.15 (q, 2H); 5.73 (s, 2H); 7.38 (m, 2H); 7.71 (m, 2H); 7.89 (m, 1H). LCMS (ESI+) for C₁₃H₁₂F₁I₁N₄O₃ m/z 419.6 (M+2H)⁺.

### Step 3: Preparation of 4-amino-3-carbamoyl-1-(2-fluoro-4-iodophenyl)-1H-pyrazole-5-carboxylic acid

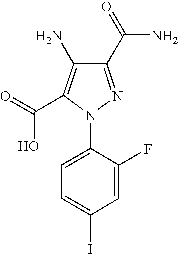

The product from Step 2 (22.5 g, 54 mM) was treated with 95% ethanol (90 mL) followed by 45 mL of 3.5 M KOH in ethanol. The reaction was stirred at rt overnight, then warmed to 46° C. for 105 min. The reaction was partially evaporated, cooled and treated with 6N HCl to pH 3. The filtered product was washed with water, and dried in vacuum oven. ¹H NMR (300 MHz, DMSO-d₆): δ 7.38 (m, 2H); 7.71 (m, 2H); 7.90 (m, 1H). LCMS (ESI+) for C₁₁H₈F₁I₁N₄O₃ m/z 391.9 (M+2H)⁺.

### Step 4: Preparation of 4-amino-1-(2-fluoro-4-iodophenyl)-1H-pyrazole-3-carboxamide

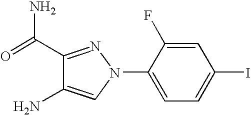

The carboxylic acid from Step 3 (1.84 g, 4.71 mmol) was treated with 10 mL 85% phosphoric acid. The reaction was heated to 68° C. for 1-1.5 h. The reaction was cooled in an ice bath and diluted with 20 mL water. The pH was adjusted to 7 by the addition of most of a solution of 11.7 g sodium hydroxide in 60 mL water, and the product was filtered, washed with water and dried under high vacuum to give a tan solid. By thin-layer chromatography (5% CH₃OH—CH₂Cl₂), the starting material appeared at the origin, while the product had an R^{f} of 0.55. ¹H NMR (300 MHz, DMSO-d₆): δ 4.90 (bs, 1H); 7.30 (s, 1H); 7.55 (m, 2H); 7.71 (m, 2H); 7.92 (m, 1H). LCMS (ESI+) for C₁₀H₈F₁₁₁N₄O₁ m/z 348.0 (M+2H)⁺.

### Step 5: Preparation of 1-(1-(4-(2-fluorophenylthio)-2-fluorophenyl)-3-carbamoyl-1H-pyrazol-4-yl)urea

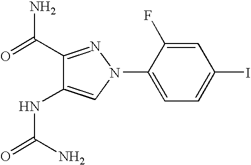

The amide from Step 4 (1.004 g, 2.90 mM) was added to a 100-mL round bottom flask and rotary evaporated from toluene in order to dry the solid and flask. N₂ was let in the flask as it was removed from the rotary evaporator, and the flask was septum-stoppered. The solid was then dissolved in 10 mL dry THF and treated with diisopropylethylamine (0.51 mL). The solution was cooled in a dry ice-CH₃OH bath under N₂. Commercial 20% phosgene in toluene (1.6 mL) was added by syringe, forming a yellow precipitate. Ammonia gas was condensed into a microware flask, allowed to evaporate through a tygon tube and recondense intod 5 mL THF at −78° C. One equivalent (0.5 mL) of diisopropylethylamine was added to the ammonia solution, and the carbamoyl chloride suspension was added to the ammonia solution. The reaction was then removed from the cold bath and stirred at rt overnight. The mixture was evaporated and treated with water, filtered, and the solid triturated with CH₂Cl₂. The solid was then dissolved in about 300 mL of CH₃QH-CH₂Cl₂ and filtered; evaporation of the CH₂Cl₂ resulted in crystallization. ¹H NMR (300 MHz, DMSO-d₆): δ 6.50 (bs, 2H); 7.54 (s, 1H); 7.70 (m, 2H); 7.80 (s, 1H); 7.91 (m, 1H); 8.41 (s, 1H); 8.66 (s, 1H). LCMS (ESI−) for C₁₁H₉F₁I₁N₅O₂ m/z 388.0 (M−H).

### Example 211

### 1-(1-(4-(2-fluorophenylthio)-2-fluorophenyl)-3-carbamoyl-1H-pyrazol-4-yl)urea

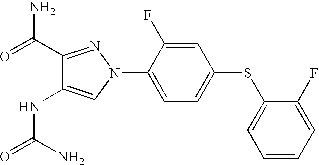

A 4 mL screw-capped vial was charged with 1-(1-(4-(2-fluorophenylthio)-2-fluorophenyl)-3-carbamoyl-1H-pyrazol-4-yl)urea (Example 210, 797 mg, 2.0 mM). To this was added potassium carbonate (569 mg, 4.1 mM), isopropanol (3 mL), and ethylene glycol (240 μL). N₂ was bubbled through the suspension. Copper (I) iodide (32.7 mg) was added, followed by 2-fluorobenzenethiol (230 μL, 2.1 mM). The vial was capped tightly and heated to 80° C. overnight. The starting material never appeared to go into solution. The reaction mixture was cooled, then worked up with CH₂Cl₂ and water. The organic layer was washed with dilute sodium hydroxide to remove any remaining thiol. Crude product was filtered from the CH₂Cl₂ layer, adsorbed onto silica gel from DMF and chromatographed on a 70 g silica column in 10% and 20% DMF-toluene. Fractions containing the desired product were evaporated and the title compound was recrystallized a number of times from minimal DMF, adding CH₃OH or water. ¹H NMR (300 MHz, DMSO-d₆): δ 6.49 (bs, 2H); 7.17 (m, 1H); 7.30 (m, 3H); 7.50 (m, 3H); 7.75 (s, 1H); 7.87 (m, 1H); 8.39 (m, 1H); 8.64 (m, 1H). LCMS (ESI+) for C₁₇H₁₃F₂N₅O₂S₁ m/z 390.0 (M+H)⁺.

### Example 212

### 4-[(aminocarbonyl)amino]-1-(4-fluorophenyl)-1H-pyrazole-3-carboxamide

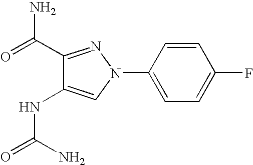

### Step 1: Preparation of N-cyano-2-[(4-fluorophenyl)hydrazono]acetamide

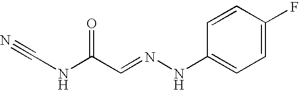

Following the procedure of Stanczak et al., Pharamzie 49, 884-89, 1994), the title material was prepared by coupling the diazonium salt from 4-fluoroaniline and cyanoacetamide in the presence of sodium acetate. The crude material was recrystallized from ethanol. C₉H₇FN₄O. MW=206.18. Calc: C, 52.43; H, 3.42; N, 27.17. Found: C, 52.20; H, 3.55; N, 27.27. mp=265-267° C.

### Step 2: Preparation of 1-(4-fluorophenyl)-3-carboxamido-4-amino-5-carboxyethylpyrazole

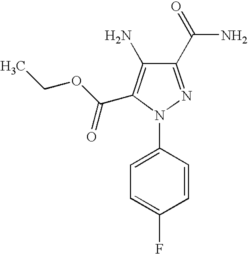

N-cyano-2-[(4-fluorophenyl)hydrazono]acetamide from Step 1 (2.06 g, 10 mmol) was dissolved in DMF (20 mL). K₂CO₃ (2.8 g, 20 mmol) and ethylbromoacetate (2.2 g 15 mmol) was added to the mixture and heated to 130° C. under N₂ for 2 h. The mixture was cooled to 90° C., excess triethylamine (2 mL) was added and the temperature maintained for 1 h. The mixture was cooled to rt, poured into ice water (160 mL), and stirred for 1 h. The mixture was cooled in the refrigerator overnight and filtered. The crude product was recrystallized from ethanol to give the title compound. C₁₃H₁₃FN₄O₃. MW=292.27. Calc: C, 53.42; H, 4.48; N, 19.17. Found: C, 53.22; H, 4.61; N, 19.00.

### Step 3: Preparation of 1-(4-fluorophenyl)-3-carboxamido-4-amino-5-carboxypyrazole

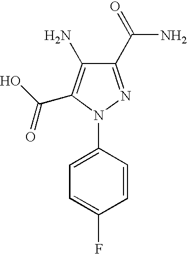

1-(4-fluorophenyl)-3-carboxamido-4-amino-5-carboxyethylpyrazole from Step 2 (1.5 g, 5 mmol) was warmed to reflux in a 20% solution of KOH (3 g) in ethanol (15 mL) for 1 h. The mixture was cooled and diluted with water (25 mL). The mixture was cooled in an ice bath and acidified to pH=4-6 with 10% HCl. The precipitate formed was filtered cold and air dried to give the title compound. C₁₁H₉FN₄O₃. MW=264.22. Calc: C, 50.00; H, 3.43; N, 21.20. Found: C, 49.60; H, 3.66; N, 20.82.

### Step 4: Preparation of 1-(4-fluorophenyl)-3-carboxamido-4-aminopyrazole

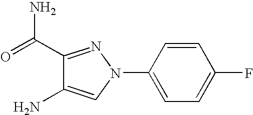

1-(4-fluorophenyl)-3-carboxamido-4-amino-5-carboxypyrazole from Step 3 (750 mg, 3 mmol) and H₃PO₄ (5 mL) were warmed to 80° C. for 45 min until evolution of CO₂ ceased. Sufficient water was added to bring the volume of the mixture to approximately 30 mL, then neutralized with K₂CO₃ to pH=8-9. The precipitate was filtered and washed with aqueous K₂CO₃. The crude material was recrystallized from ethanol to give the title compound. C₁₀H₉FN₄OMW=220.21. Calc: C, 54.54; H, 4.12; N, 25.44. Found: C, 54.18; H, 4.38; N, 25.01.

### Step 5: Preparation of 4-[(aminocarbonyl)amino]-1-(4-fluorophenyl)-1H-pyrazole-3-carboxamide

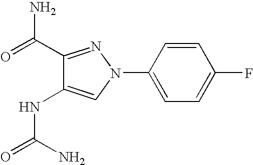

1-(4-fluorophenyl)-3-carboxamido-4-aminopyrazole from Step 4 (220 mg, 1 mmol) was stirred in a mixture of acetic acid (4 mL) and water (1 mL). NaCNO (275 mg, 4 mmol) was added and the contents stirred 3 days at rt. The contents were diluted with water (25 mIs) and filtered. The crude material was recrystallized from ethanol/water to give the title compound. C₁₁H₁₀FN₅O₂. MW=263.23. Calc: C, 50.19; H, 3.83; N, 26.61. Found: C, 50.01; H, 3.99; N, 26.13.

### Example 213

### 4-[(aminocarbonyl)amino]-1-(1,3-benzodioxol-5-yl)-1H-pyrazole-3-carboxamide

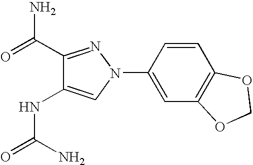

Prepared according to the procedure of Example 212. C₁₂H₁₁N₅O₄H₂O. MW=307.27. Calc: C, 46.91; H, 4.26; N, 22.79. Found: C, 46.55; H, 4.33; N, 22.16.

### Example 214

### 4-[(aminocarbonyl)amino]-1-(4-chlorophenyl)-1H-pyrazole-3-carboxamide

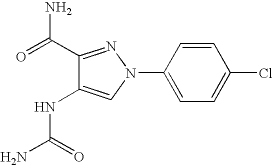

### Step 1: Preparation of (2E)-3-(aminooxy)-2-[(4-chlorophenyl)hydrazono]-3-oxopropanen itrile

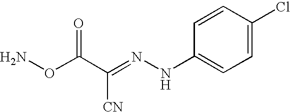

A stirred mixture of 4-chlorobenzenamine (12.8 g, 100 mmol), conc. HCl (25 mL) and water (75 mL) at 0-5C was added portion-wise to a solution of NaNO₂ (6.9 g, 100 mmol) in water (20 mL) over 30 min. NaOAc (25 g) in water (100 mL) was added at 0° C., followed by portion-wise addition of a mixture of cyanoacetamide (8.4 g, 100 mmol), NaOAc (25 g), ethanol (100 mL) and water (100 mL). The mixture was stored at −20° C. overnight, filtered, washed with water (5×100 mL), and dried over 60° C. in a vacuum to give the title compound as a yellow solid. LCMS (M+1)=254.

### Step 2: Preparation of ethyl 4-amino-3-(aminocarbonyl)-1-(4-chlorophenyl)-1H-pyrazole-5-carboxylate

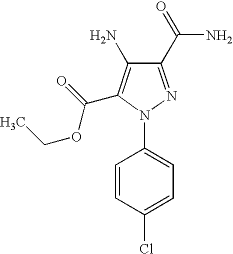

A mixture of (2E)-3-(aminooxy)-2-[(4-chlorophenyl)hydrazono]-3-oxopropanenitrile from Step 1 (11.15 g, 50 mmol), bromo ethylacetate (8.35 g, 50 mmol), K₂CO₃ (13.8 g, 100 mmol) in DMF (100 mL) was heated to 130° C. over 2 h. The mixture was cooled to 90° C., TEA (20 mL) was added and the mixture stirred for 2 h. The mixture was cooled, water (150 mL) added and filtered, washed with water, and air dry to give the title compound as a brown solid. ¹H NMR, LCMS (M+1=310) confirmed its structure.

### Step 3: Preparation of 4-amino-1-(4-chlorophenyl)-1H-pyrazole-3-carboxamide

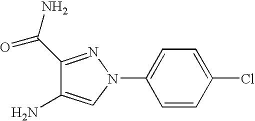

A mixture of the ester from Step 2 (6.18 g, 20 mmol) and KOH (11 g) in EtOH (100 mL) was heated to reflux for 2 h. The mixture was cooled to 0° C., and ice-water (100 mL) and conc. HCl (17 mL) were added. The mixture was filtered, washed with water, and air dry to give the acid. This acid (1.41 g, 5 mmol) was heated with LiI (0.66 g, 25 mmol) in DMF (10 mL) to 150° C. for 6 h. The mixture was cooled and filtered. The DMF solution was added to 30 mL water, filtered to give a yellow solid with excellent ¹H NMR, LCMS, CNH profile. Calculated for C₁₀H₉ClN₄O: C, 50.75; H, 3.83; N, 23.67. Found C, 50.12; H, 3.88; N, 23.11.

### Step 4: Preparation of 4-[(aminocarbonyl)amino]-1-(4-chlorophenyl)-1H-pyrazole-3-carboxamide

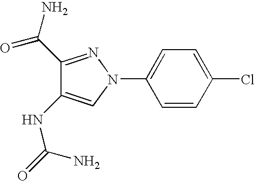

A mixture of the amide from Step 3 (319 mg, 1.35 mmol) in CH₃CN (8 mL) at rt was added to trichloroacetyl isocyanate (267 mg, 1.41 mmol), stirred for 2 h, and NH₃ in CH₃OH (2 M, 10 mL) was added. After stirring overnight, the mixture was filtered, the solid washed with ethanol and air dried to give the title compound as an off white solid with excellent ¹H NMR and LCMS profiles. ¹H NMR (400 MHz, DMSO-d₆): δ 6.50 (s, 2H) 7.50 (m, 2H) 7.54 (d, J=8.86 Hz, 2H) 7.91 (d, J=8.86 Hz, 2H) 8.56 (s, 1H) 8.66 (s, 1H). LC MS (M+H): 280.18. Calculated for C₁₁H₁₀ClN₅O₂(H₂O)₀₃: C, 46.34; H, 3.75: N, 24.57. Found C, 46.68; H, 3.56; N, 24.40.

### Example 215

### 4-[(aminocarbonyl)amino]-1-(4-iodophenyl)-1H-pyrazole-3-carboxamide

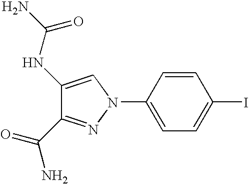

Prepared according to the same procedure as Example 214.

### Example 216

### 4-[(aminocarbonyl)amino]-1-(4-isopropylphenyl)-1H-pyrazole-3-carboxamide

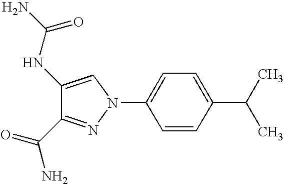

A suspension of 4-[(aminocarbonyl)amino]-N-(2,4-dimethoxybenzyl)-1H-pyrazole-3-carboxamide (Example 209, Step 4,100 mg, 0.31 mmol) in DMF (5.0 mL) was treated with p-isopropylbenzeneboronic acid (61 mg, 0.37 mmol) and Cu(I)Cl (6.1 mg, 0.062 mmol). Pyridine (0.047 mL, 0.465 mmol) was added to the mixture. The reaction mixture was stirred in open air overnight at 80° C. DMF was evaporated under pressure. The crude mixture was stirred in neat trifluoroacetic acid (2 mL) for 10 min, evaporated, re-dissolved in DMF, and filtered through a syringe filter (0.45 μm) and purified by prep rpHPLC, and lyophilized to give the title compound as a white solid. ¹H NMR (400 MHz, DMSO-d₆): δ 1.19 (s, 3H) 1.21 (s, 3H) 2.91 (m, 1H) 6.48 (m, 2H) 7.34 (m, 2H) 7.47 (m, 1H) 7.69 (m, 1H) 7.75 (m, 2H) 8.49 (s, 1H) 8.63 (s, 1H). ESI Mass Spectrum for C₁₄H₁₈N₅O₂⁺: 288.1 (M+H).

Examples 217-219 were prepared according to the same procedure as Example 216.

| | | ¹H NMR | MS |
|---|---|---|---|
| Example | Name and Structure | (500 MHz, CD₃OD) | (M + H) |
| 217 | 4-[(aminocarbonyl)amino]-1-(1H-indol-5-yl)- | δ 6.56(d, 1H), 7.36(d, 1H), 7.51 | 285.10 |
| | 1H-pyrazole-3-carboxamide | (d, 1H), 7.55(dd, 1H), 7.93(d, 1 | |
| | 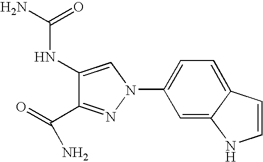 | H), 8.46(s, 1H) | |
| 218 | 4-[(aminocarbonyl)amino]-1-(3,4- | δ 7.43(m, 1H), 7.65(m, 1H), | 282.1 |
| | difluorophenyl)-1H-pyrazole-3-carboxamide | 7.88(m, 1H), 8.52(s, 1H) | |
| | 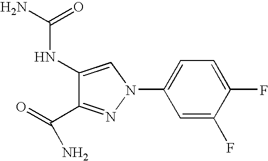 | | |
| 219 | 4-[(aminocarbonyl)amino]-1-(2-naphthyl)- | | 296.1 |
| | 1H-pyrazole-3-carboxamide | | |
| | 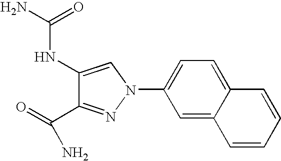 | | |

### Example 220

### 4-[(aminocarbonyl)amino]-1-[2-chloro-3′-(cyanomethoxy)-1,1′-biphenyl-4-yl]-1H-pyrazole-3-carboxamide

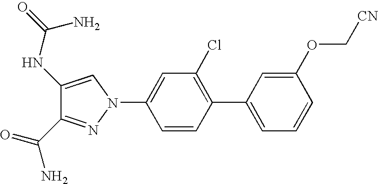

To a flask were added 4-[(aminocarbonyl)amino]-1-(2-chloro-3′-hydroxy-1,1′-biphenyl-4-yl)-1H-pyrazole-3-carboxamide (Example 7, 0.087 g, 0.23 mmol), DMF (5 mL, dry), K₂CO₃ (0.097 g, 0.7 mmol); the mixture was then flushed with vacuum/N₂ and then acetone (1 mL) and bromoacetonitrile (0.04 mL, 57 mmol) were added. The mixture was stirred under N₂. After overnight stirring, the mixture was evaporated to a dark, oily solid, stirred with H₂O (20 mL), filtered and the filtered brown solid washed with H₂O (5×5 mL) to give a brown solid. Trituration with CH₂Cl₂(4×5 ml) gave the title compound as a light brown solid. ¹H NMR (300 MHz, DMSO-d₆): δ 8.66 (brs, 2H), 8.18 (brs, 1H), 7.92 (m, 2H), 7.50 (m, 3H), 7.14 (m, 3H), 6.52 (brs, 2H), 5.21 (brs, 2H). MS (ESI−) for C₁₉H₁₅ClN₆O₃: m/z 409.1 (M−H)⁻.

### Example 221

### 4-[(aminocarbonyl)amino]-1-{3-chloro-4-[(4-fluorophenyl)thio]phenyl}-1H-pyrazole-3-carboxamide

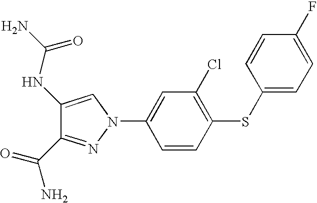

A stirring solution of 4-[(aminocarbonyl)amino]-1-(3-chloro-4-iodophenyl)-1H-pyrazole-3-carboxamide (Example 6, 0.610 g, 1.5 mmol), CuI (0.29 g, 1.5 mmol), N,N-diisopropylethylamine (5 ml), ethylene glycol (1 mL, 75 mmol), 4-fluorothiophenol (0.20 mL, 4.5 mmol), and DMF (10 mL) was placed under vacuum for 20 min and then placed under N₂. The stirring mixture was then immersed in an oil bath (105° C.). After 3.5 h, the heat was turned off and the mixture stirred overnight. The mixture was then poured onto ice (100 mL) plus Na₂CO₃ (0.3 g in 10 mL H₂O); after stirring for 1 h, the mixture was filtered to give a brown solid. This solid was triturated with CH₂Cl₂ (5×20 mL) to give the title compound as a light brown solid. ¹H NMR (300 MHz, DMSO-d₆): δ 8.65 (s, 1H), 8.57 (m, 1H), 8.18 (m, 1H), 7.88 (brs, 1H), 7.80 (m, 1H), 7.53 (m, 3H), 7.30 (m, 2H), 7.03 (m, 1H), 6.51 (brs, 2H). MS (ESI−) for C₁₇H₁₃ClFN₅O₂S m/z 404.0 (M−H)⁻.

### Example 222

### 4-[(aminocarbonyl)amino]-1-{3-chloro-4-[(4-fluorophenyl)sulfonyl]phenyl}-1H-pyrazole-3-carboxamide

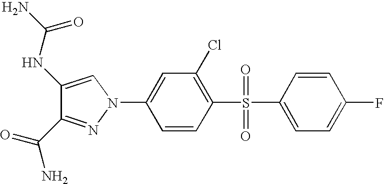

To a flask containing 4-[(aminocarbonyl)amino]-1-{3-chloro-4-[(4-fluorophenyl)thio]phenyl}-1H-pyrazole-3-carboxamide (Example 221, 0.220 g, 0.54 mmol) and meta-chloroperbenzoic acid (0.617 g, about 77%, 2.7 mmol) was added CH₂Cl₂ (10 mL). After overnight stirring, more meta-chloroperbenzoic acid (0.29 g, 1.6 mmol) was added. After the reaction stirred for five days, Na₂CO₃ (1 g, 10 mmol, in 30 mL H₂O) was added to the heterogeneous reaction mixture; after stirring another hour, the mixture was filtered through a sintered glass funnel (5×10 mL H₂O washes), the filtered solid air dried 3 h and then placed under high vacuum for 2 days to give the title compound as a cream-colored solid. ¹H NMR (300 MHz, DMSO-d₆): δ 8.70 (m, 2H), 8.30 (m, 2H), 8.17 (m, 1H), 8.01 (m, 3H), 7.63 (s, 1H), 7.45 (m, 2H), 6.57 (brs, 2H). MS (ESI−) for C₁₇H₁₃ClFN₅O₄S m/z 435.9 (M−H)⁻.

### Example 223

### 4-[(aminocarbonyl)amino]-1-(2-chloro-2′-hydroxy-1,1′-biphenyl-4-yl)-1H-pyrazole-3-carboxamide

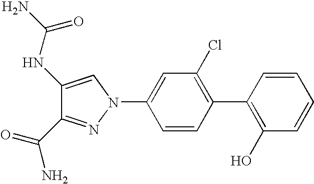

To a 2-neck flask were added a stir bar, 4-[(aminocarbonyl)amino]-1-(3-chloro-4-iodophenyl)-1H-pyrazole-3-carboxamide (Example 6, 0.302 g, 0.745 mmol), tetrakis(triphenylphosphine)rhodium(0) (0.086 g, 0.0745 mmol), 2-hydroxyphenylboronic acid (0.205 g, 1.5 mmol), and dry DMF (5 mL). This stirring solution was flushed with vacuum/N₂ for 10 min. Cs₂CO₃ (0.588 g, 1.8 mmol) was mixed with H₂O (0.9 mL) and N₂ was bubbled through the resultant solution for 10 min. The solution was then added by syringe to the reaction mixture above which was then immersed in an oil bath (80° C.). After overnight stirring, the heat bath was removed and, after 10 min, the mix was poured onto ice (100 mL), stirred 20 min, and filtered (4×30 ml H₂O washes). A greyish tan solid was isolated; this solid was triturated with CH₂Cl₂ (50 mL) and air-dried to give the title compound as a light brown solid. ¹H NMR (300 MHz, DMSO-d₆): δ 9.62 (brs, 1H), 8.68 (m, 1H), 8.63 (m, 1H), 8.10 (m, 1H), 7.86 (m, 2H), 7.54 (brs, 1H), 7.40 (m, 1H), 7.19 (m, 1H), 7.10 (m, 1H), 6.89 (m, 2H), 6.53 (brs, 2H). MS (ESI−) for C₁₇H₁₄ClN₅O₃ m/z 370.1 (M−H)⁻.

### Example 224

### 4-[(aminocarbonyl)amino]-1-[2-chloro-2′-(cyanomethoxy)-1,1′-biphenyl-4-yl]-1H-pyrazole-3-carboxamide

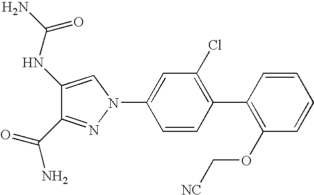

To a stirring solution of 4-[(aminocarbonyl)amino]-1-(2-chloro-2′-hydroxy-1,1′-biphenyl-4-yl)-1H-pyrazole-3-carboxamide (Example 223, 0.080 g, 0.22 mmol) in DMF (4 mL) and acetone (1 mL) was added bromoacetonitrile (0.030 mL, 0.43 mmol) and K₂CO₃ (0.094 g, 0.65 mmol). After stirring for 20 h, the reaction mixture was evaporated to a dark, oily solid which was stirred with H₂O (15 mL) for 60 min then filtered (4×15 mL H₂O washes) and air dried to give the title compound as a brown solid. ¹H NMR (300 MHz, DMSO-d₆): δ 8.66 (m, 2H), 8.14 (brs, 1H), 7.88 (m, 2H), 7.50 (m, 3H), 7.21 (m, 3H), 6.53 (brs, 2H), 5.15 (s, 2H). MS (ESI−) for C₁₉H₁₅ClN₆O₃ m/z 408.9 (M−H)⁻.

### Example 225

### 4-[(aminocarbonyl)amino]-1-(4-bromo-3-fluorophenyl)-1H-pyrazole-3-carboxamide

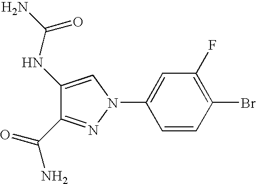

### Step 1: Preparation of 2-[(4-bromo-3-fluorophenyl)hydrazono]-2-cyanoacetamide

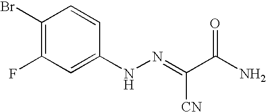

To a 500 mL flask was added 3-fluoro-4-bromoaniline (23.902 g, 190.0 mmol) and a stir bar; the flask cooled to −4° C. in acetone/ice bath. To this was added conc. HCl (31.5 ml) dropwise over a period of 5 min, the temperature staying below 5° C. To this was added H₂O (100 mL) in 3 portions over 3 min, temperature rising to 22° C. before dropping back below 5° C. To this slurry was added a solution of NaNO₂ (8.68 g, 69 mmol) in H₂O (25 mL plus a 3 mL rinse) over a 5 min period, the temp staying below 5° C. (10 mL ice was added to aid in temp control; a little more liquid also appeared to help with stirrability of the mixture). This mixture was a heterogeneous yellow/light brown. After 90 min, a cooled solution of NaOAc3H₂O (51.4 g, 136.1 mmol) in H₂O (125 mL) was added over a 5 min period, the temp staying below 5C. The resultant yellowish slurry was then used as described in the following paragraph.

A stirring solution of cyanoacetamide (21.15 g, 251 mmol)), and NaOAC 3H₂O (17.1 g, 208 mmol) in H₂O (2400 mL) and ethanol (125 mL) in a 1000-mL beaker was cooled in an acetone/water bath. To this stirring solution was added (dropwise over a 75-min period) the slurry from the previous paragraph, a mostly bright yellow(heterogeneous) mixture resulting and becoming difficult to stir. 40 min later, the still cold (5° C.) mixture was filtered (sintered glass funnel, 5×200 ml H₂O rinses), air-dried 10 min, and dried on rotary evaporator. The solid material was triturated with CH₃OH (6×150 mL) until orange color no longer came off. Air drying gave the title compound as a bright yellow solid. ¹H NMR (300 MHz, DMSO-d₆): δ 11.83 (s, 1H), 7.98 (s, 1H), 7.80 (m, 1H), 7.68 (m, 2H), 7.36 (m, 1H). MS (ESI−) for C₉H₆BrFN₄O m/z 285.0 (M−H)⁻.

### Step 2: Preparation of ethyl 4-amino-3-(aminocarbonyl)-1-(4-bromo-3-fluorophenyl)-1H-pyrazole-5-carboxylate

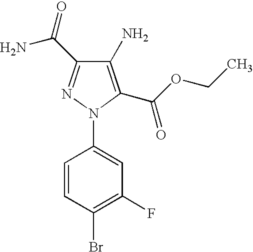

A stirring mixture of the cyanoacetamide from Step 1 (33.00 g, 116 mmol), potassium carbonate (16 g, 116 mmol), ethyl bromoacetate (19 mL, 174 mmol), and DMF (250 mL) under N₂ was immersed in an oil bath (80° C.). After 3 hrs, more ethyl bromoacetate (10 mL, 87 mmol) and K₂CO₃ (1.0 g, 7 mmol) were added. After 30 min, Et₃N (15 mL, 108 mmol) was added to the mixture; after 90 more min, the heat was turned off, the stirring mixture being allowed to gradually cool to rt overnight. The red mixture was poured onto ice (500 mL), stirred and then allowed to sit for 2 h, followed by filtration through a sintered glass funnel (5×200 mL H₂O washes) to give a reddish-brown solid which was triturated with CH₂Cl₂ (3×50 mL) to give the title compound as a white solid. ¹H NMR (300 MHz, DMSO-d₆): δ 7.79 (m, 1H), 7.66 (m, 2H), 7.39 (brs, 1H), 7.32 (m, 1H), 5.68 (m, 2H), 4.15 (m, 2H), 1.12 (m, 3H). MS (ESI+) for C₁₃H₁₂BrFN₄O₃ m/z 371.9 (M+H)⁺.

### Step 3: Preparation of 4-amino-3-(aminocarbonyl)-1-(4-bromo-3-fluorophenyl)-1H-pyrazole-5-carboxylic acid, potassium salt

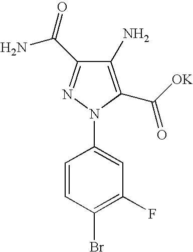

A stirring mixture of the ester from Step 2 (33.81 g, 91.1 mmol), KOH (6.64 g, 118.0 mmol), and ethanol (180 mL, anhydrous), was immersed in a water bath at 80° C. After 3.5 h, the mixture was removed from heat and filtered (still warm), washed with CH₃OH (2×200 mL), and air dried to give the title compound as a white solid. ¹H NMR (300 MHz, DMSO-d₆): δ 7.62 (m, 1H), 7.50 (m, 1H), 7.29 (m, 2H), 7.09 (brs, 1H), 5.33 (brs, 2H). MS (ESI+) for C₁₁H₇BrFKN₄O₃ m/z 344.0 (M+H)⁺.

### Step 4: Preparation of 4-amino-1-(4-bromo-3-fluorophenyl)-1H-pyrazole-3-carboxamide

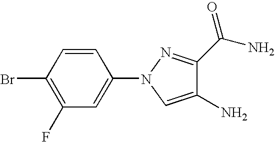

The potassium salt from Step 3 (26.53 g, 70 mmol) was placed in a 500-mL flask; to this was added H₃PO₄ (200 mL) and the stirring mixture immersed in oil bath (80° C.). At 4 h, more H₃PO₄ (50 mL) added. At 5 h; the reaction mixture was removed from heat, poured onto ice (800 mL) and filtered, washed with H₂O (5×150 mL), sat'd aqueous sodium bicarbonate (100 mL), and H₂O (3×100 ml), and then air-dried to give the title compound as an off-white solid. ¹H NMR (300 MHz, DMSO-d₆): δ 7.93 (m, 1H), 7.79 (m, 2H), 7.64 (m, 2H), 7.27 (m, 1H), 4.90 (brs, 2H). MS (ESI+) for C₁₀H₈BrFN₄O m/z 300.1 (M+H)⁺.

### Step 5: Preparation of 4-[(aminocarbonyl)amino]-1-(4-bromo-3-fluorophenyl)-1H-pyrazole-3-carboxamide

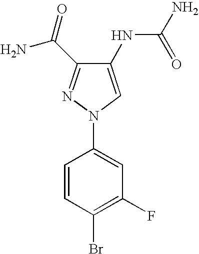

A mixture of 4-amino-1-(4-bromo-3-fluorophenyl)-1H-pyrazole-3-carboxamide from Step 4 (5.33 g, 18 mmol), 70% acetic acid (220 mL) and 2-propanol (330 mL) was stirred at rt. To this was added a solution of potassium cyanate (1.73 g, 21 mmol) in H₂O (330 mL). After overnight stirring, more KOCN (1 g, 12 mmol, in 5 mL H₂O) was added. After a second night, more KOCN (1 g, 12 mmol, in 5 mL H₂O) was added. After a third night, the mixture was diluted with H₂O (100 mL) and filtered (4×150 mL H₂O washes), air-dried to give the title compound as a white solid. ¹H NMR (300 MHz, DMSO-d₆): δ 8.64 (m, 2H), 8.03 (m, 1H), 7.67 (m, 3H), 7.56 (m, 1H), 6.53 (brs, 2H). MS (ESI−) for C₁₁H₉BrFN₅O₂ m/z 340.0 (M−H)⁻.

### Example 226

### 4-[(aminocarbonyl)amino]-1-(2-fluoro-3′-hydroxy-1,1′-biphenyl-4-yl)-1H-pyrazole-3-carboxamide

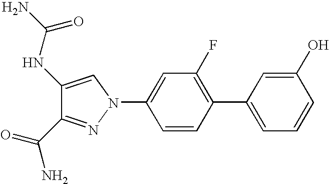

To a 2-neck flask were added a stir bar, 4-[(aminocarbonyl)amino]-1-(4-bromo-3-fluorophenyl)-1H-pyrazole-3-carboxamide (Example 225, 0.558 g, 1.6 mmol), tetrakis(triphenylphosphine)palladium(0) (0.188 g, 0.16 mmol), 3-hydroxyphenylboronic acid (0.450 g, 3.3 mmol), and dry DMF (10 mL) via syringe; this stirring solution was flushed with vacuum/N₂. Cs₂CO₃ (1.29 g, 3.9 mmol) was mixed with H₂O (1.8 mL) and N₂ was bubbled through the resultant solution for 10 min. The solution was then added by syringe to the reaction mixture above which was then immersed in an oil bath (80° C.). After overnight stirring, the heat bath was removed and after 10 min, the reaction mixture was poured onto ice (200 ml), stirred 30 min, and filtered (4×30 ml H₂O washes); the filtered solid was air-dried to give an off-white solid. This off-white solid was stirred with CH₂Cl₂(3×30 mL) and filtered to give the title compound as an ivory solid. ¹H NMR (300 MHz, DMSO-d₆): δ 9.60 (m, 1H), 8.67 (m, 2H), 7.84 (m, 3H), 7.54 (m, 2H), 7.24 (m, 1H), 6.96 (m, 2H), 6.78 (m, 1H), 6.54 (brs, 2H). MS (ESI−) for C₁₇H₁₄FN₅O₃ m/z 354.1 (M−H)⁻.

### Example 227

### 4-[(aminocarbonyl)amino]-1-[3′-(cyanomethoxy)-2-fluoro-1,1′-biphenyl-4-yl]-1H-pyrazole-3-carboxamide

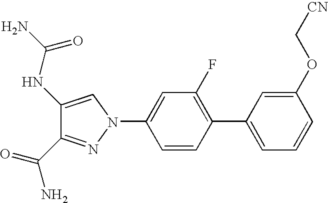

To a stirring solution of 4-[(aminocarbonyl)amino]-1-(2-fluoro-3′-hydroxy-1,1′-biphenyl-4-yl)-1H-pyrazole-3-carboxamide (Example 226, 0.186 g, 0.52 mmol) in DMF (10 mL) and acetone (2 mL) was added bromoacetonitrile (0.070 mL, 1.0 mmol) and K₂CO₃ (0.207 g, 1.5 mmol). The reaction was stirred three days and then more K₂CO₃ (0.020 g, 0.14 mmol) was added, followed 1 h later by more bromoacetonitrile (0.010 mL, 0.14 mmol). After another night of stirring, the mixture was evaporated to a brown solid, mixed with water (30 mL), stirred 30 min and filtered (5×5 mL H₂O washes) to give a dark brown solid. This solid was washed sequentially with 2-propanol (3×5 mL), ethanol (95%, 3×5 mL), CH₃OH (3×5 mL), and CH₂Cl₂ (3×5 mL); air drying gave the title compound as a light brown solid. ¹H NMR (300 MHz, DMSO-d₆): δ 8.67 (brs, 2H), 7.90 (m, 3H), 7.58 (m, 3H), (, H), 7.26 (m, 2H), 7.11 (m, 1H), 6.53 (brs, 2H), 5.20 (m, 2H). MS (ESI−) for C₁₉H₁₅FN₆O₃ m/z 393.1 (M−H)⁻.

### Example 228

### 4-[(aminocarbonyl)amino]-1-{4-[(4-fluorophenyl)thio]-2-methylphenyl}-1H-pyrazole-3-carboxamide

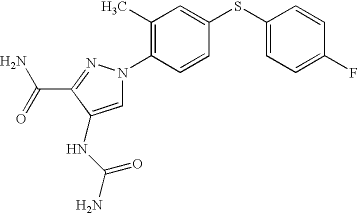

To a mixture of 1-(4-iodo-2-methyl-phenyl)-4-ureido-1H-pyrazole-3-carboxylic acid amide (Example 4, 501 mg, 1.30 mmol) and cuprous iodide (248 mg, 1.30 mmol) was added dry DMF (8 mL). The mixture was placed under N₂ and ethylene glycol (364 UL, 404 mg, 6.50 mmol), di-isopropylethylamine (1.13 mL, 841 mg, 6.50 mmol) and 4-fluorothiophenol (279 μL, 333 mg, 2.60 mmol) was added. The reaction was then heated at 105° C. for 5 h, cooled and poured into ice cold aqueous 1% sodium hydroxide solution. Filtration afforded a dirty yellow solid which was washed with CH₃OH (12 mL), ether (12 mL), acetone (15 mL) and hexane (10 mL). The resulting solid was digested with a boiling mixture (50 mL) of CHCl₃-CH₃OH-acetone, cooled and filtered to afford the sulfide as a yellow solid. ¹H NMR (300 MHz, DMSO-d₆): δ 2.16 (s, 3H), 6.45 (s, 2H), 7.12 (d, J=7.8 Hz, 1H), 7.35 (m, 7H), 7.63 (s, 1H), 8.10 (s, 1H), 8.65 (s, 1H); LCMS: m+=386, m−=384.

### Example 229

### 4-[(aminocarbonyl)amino]-1-{4-[(4-fluorophenyl)sulfonyl]-2-methylphenyl}-1H-pyrazole-3-carboxamide

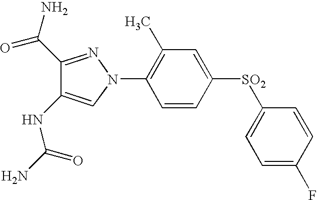

To a stirred suspension of 4-[(aminocarbonyl)amino]-1-{4-[(4-fluorophenyl)thio]-2-methylphenyl}-1H-pyrazole-3-carboxamide (Example 228,127 mg, 0.330 mmol) in CH₂Cl₂ (7 mL) was added m-chloroperbenzoic acid (227 mg, 1.32 mmol) After 18 h additional m-chloroperbenzoic acid (124 mg, 0.72 mmol) was added. After 1 h the reaction was filtered and the precipitate washed with CH₂Cl₂ (10 mL) and a 1% aqueous solution of sodium hydroxide (12 mL). The organic layer was evaporated and the resulting solid washed with 1% aqueous sodium hydroxide (20 mL) and water (10 mL). The resulting solid was chromatographed over silica gel (5% CH₃OH—CHCl₃) to afford the sulfone as a yellow solid. ¹H NMR (300 MHz, DMSO-d₆): δ 2.36 (s, 3H), 6.47 (s, 2H), 7.46 (m, 3H), 7.65 (m, 2H), 7.88 (d, J=8.1 Hz, 1H), 8.06 (m, 3H), 8.24 (s, 1H), 8.66 (s, 1H); LCMS: m+=418, m−=416.

### Example 230

### 4-[(aminocarbonyl)amino]-1-(2-ethoxy-3′-hydroxy-1,1′-biphenyl-4-yl)-1H-pyrazole-3-carboxamide

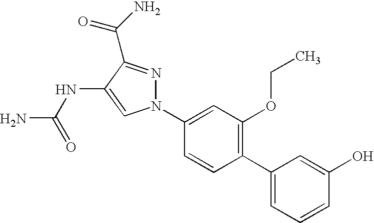

To a suspension of 1-(4-bromo-3-ethoxyphenyl)-4-ureido-1H-pyrazole-3-carboxylic acid amide (517 mg, 1.40 mmol, prepared according to the process of Example 3), 3-hydroxyphenylboronic acid (387 mg, 2.81 mmol) and tetrakis(triphenylphosphine)palladium(0) (243 mg, 0.211 mmol) in dry DMF (10 mL) under N₂ was added a solution of Cs₂CO₃ (1.37 g, 4.21 mmol) in water (2.5 mL). The stirred mixture was heated at 80° C. for 16 h, cooled and poured into ice-water (100 mL). The precipitate was filtered, washed with water, dried then dissolved in hot 10% CH₃OH—CHCl₃ and filtered through a plug of silica gel. The product was obtained as a sand-colored solid. ¹H NMR (300 MHz, DMSO-d₆): δ 1.29 (t, J=6.9 Hz, 3H), 4.15 (q, J=6.9 Hz, 2H), 6.51 (s, 2H), 6.69 (m, 1H), 6.93 (m, 2H), 7.17 (m, 1H), 7.35 (m, 1H), 7.46 (m, 1H), 7.54 (s, 1H), 7.59 (s, 1H), 7.86 (s, 1H), 8.60 (s, 1H), 8.66 (s, 1H), 9.38 (s, 1H); LCMS: m+=382 and m−=380.

### Example 231

### 4-[(aminocarbonyl)amino]-1-[3′-(cyanomethoxy)-2-ethoxy-1,1′-biphenyl-4-yl]-1H-pyrazole-3-carboxamide

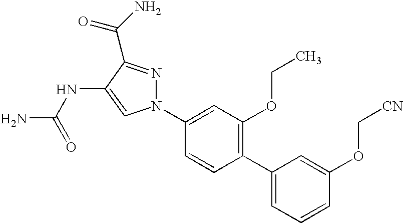

Bromoacetonitrile (100 μL, 180 mg, 1.50 mmol) was added to a stirred suspension of 4-[(aminocarbonyl)amino]-1-(2-ethoxy-3′-hydroxy-1,1′-biphenyl-4-yl)-1H-pyrazole-3-carboxamide (Example 230,192 mg, 0.503 mmol) and Cs₂CO₃ (328 mg, 1.01 mmol) in dry DMF (6.0 mL) and stirred under N₂ for 40 h. The solvent was evaporated and the residue treated with water (30 mL) then filtered. The residue was chromatographed over silica (5% CH₃OH—CHCl₃) to afford the product as a white solid. ¹H NMR (300 MHz, DMSO-d₆): δ 1.31 (t, J=6.5 Hz, 3H), 4.18 (q, J=6.5 Hz, 2H), 5.19 (s, 2H), 6.51 (s, 2H), 7.00 (m, 1H), 7.24 (s, 2H), 7.40 (m, 2H), 7.50 (m, 2H), 7.62 (s, 1H), 7.87 (s, 1H), 8.62 (s, 1H), 8.67 (s, 1H); LCMS: m+=421 and m−=419.

### Example 232

### 4-[(aminocarbonyl)amino]-1-(2-ethoxy-2′-hydroxy-1,1′-biphenyl-4-yl)-1H-pyrazole-3-carboxamide

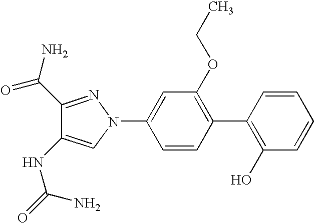

To a stirred mixture of 1-(4-bromo-3-ethoxyphenyl)-ureido-1H-pyrazole-3-carboxylic acid amide (485 mg, 1.32 mmol), 2-hydroxyphenylboronic acid (363 mg, 2.63 mmol) and tetrakis(triphenylphosphine)palladium(0) (228 mg, 0.198 mmol) in dry DMF (10 mL) under N₂ a solution of Cs₂CO₃ (1.29 g, 3.95 mmol) in water (2.5 mL) was added. The mixture was heated at 80° C. for 15 h then cooled and poured into water (80 mL). Filtration gave a brown solid which was chromatographed over silica gel (4% CH₃OH—CHCl₃) to afford the product as a cream solid. ¹H NMR (300 MHz, DMSO-d₆): δ 1.21 (t, J=6.9 Hz, 3H), 4.11 (q, J=6.9 Hz, 2H), 6.48 (s, 2H), 6.79 (m, 1H), 6.86 (m, 1H), 7.11 (m, 2H), 7.24 (d, J=7.8 Hz, 1H), 7.40 (m, 1H), 7.53 (m, 2H), 7.81 (s, 1H), 8.58 (s, 1H), 8.66 (s, 1H), 9.17 (s, 1H); LCMS: m+=382, m−=380.

Examples 233-235 were prepared according to the same procedure as Example 232.

| | | ¹H NMR | |
|---|---|---|---|
| Example | Name and Structure | (300 MHz, DMSO-d₆) | LCMS |
| 233 | 4-[(aminocarbonyl)amino]-1-[3′-hydroxy-2- | δ 6.55(s, 2H), 6.72(m, 2H), | M + = 406 |
| | (trifluoromethyl)-1,1′-biphenyl-4-yl]-1H- | 6.80(d, J=8.4Hz, 1H), 7.24(m, | M − = 404 |
| | pyrazole-3-carboxamide | 1H), 7.47(d, J=8.1Hz, 1H), | |
| | 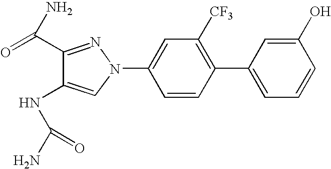 | 7.57(s, 1H), 7.98(s, 1H), 8.17 (d, J=7.8Hz, 1H), 8.32(s, 1H), 8.71(m, 2H), 9.59(s, 1H) | |
| 234 | 4-[(aminocarbonyl)amino]-1-[2′-hydroxy-2- | δ 6.54(s, 2H), 6.81(m, 1H), | M + = 406 |
| | (trifluoromethyl)-1,1′-biphenyl-4-yl]-1H- | 6.89(d, J=7.2Hz, 1H), 7.04(m, | M − = 404 |
| | pyrazole-3-carboxamide | 1H), 7.20(m, 1H), 7.40(d, | |
| | 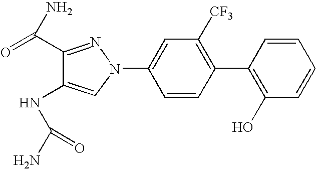 | J=7.8Hz, 1H), 7.56(s, 1H), 7.96(s, 1H), 8.13(d, J=7.8Hz, 1H), 8.29(s, 1H), 8.71(s, 2H), 9.48(s, 1H) | m+ = 406 m− = 404 |
| 235 | 4-[(aminocarbonyl)amino]-1-(2′-hydroxy-3- | δ 3.31(s, 2H), 6.46(s, 2H), | M + = 352 |
| | methyl-1,1′-biphenyl-4-yl)-1H-pyrazole-3- | 6.90(m, 2H), 7.16(m, 1H), | M − = 350 |
| | carboxamide | 7.28(d, J=7.5Hz, 1H), 7.45(m, | |
| | 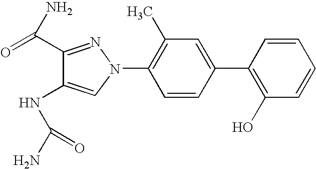 | 5H), 7.63(s, 1H), 8.14(s, 1H), 8.68(s, 1H), 9.61(s, 1H) | |

### Example 236

### 4-[(aminocarbonyl)amino]-1-[2′-(cyanomethoxy)-2-ethoxy-1,1′-biphenyl-4-yl]-1H-pyrazole-3-carboxamide

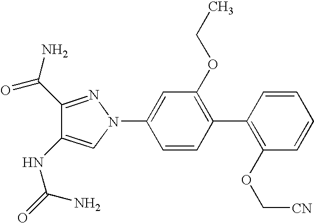

Bromoacetonitrile (78 μL, 141 mg, 1.17 mmol) was added to a stirred mixture of 4-[(aminocarbonyl)amino]-1-(2-ethoxy-2′-hydroxy-1,1′-biphenyl-4-yl)-1H-pyrazole-3-carboxamide (Example 232,149 mg, 0.391 mmol) and anhydrous potassium carbonate (108 mg, 0.781 mmol) in dry DMF (4 mL). Stirring was continued for 60 h then the solvent evaporated and water (25 mL) added to the residue. Filtration gave a brown solid which was chromatographed over silica gel (4% CH₃OH—CHCl₃) to afford the product as a cream solid. ¹H NMR (300 MHz, DMSO-d₆): δ 1.21 (t, J=6.9 Hz, 3H), 4.12 (q, J=6.9 Hz, 2H), 5.09 (s, 2H), 6.50 (s, 2H), 7.20 (m, 4H), 7.40 (m, 2H), 7.55 (m, 2H), 7.85 (s, 1H), 8.60 (s, 1H), 8.66 (s, 1H); LCMS: m+=421, m−=419.

Examples 237 and 238 were prepared according to the same procedure as Example 236.

| | | ¹H NMR | |
|---|---|---|---|
| Example | Name and Structure | (300 MHz, DMSO-d₆) | LCMS |
| 237 | 4-[(aminocarbonyl)amino]-1-[3′-(cyanomethoxy)- | δ 5.20(s, 2H), 6.54(s, 2 | m + = 445 |
| | 2-(trifluoromethyl)-1,1′-biphenyl-4-yl]-1H- | H), 7.04(s, 2H), 7.13(d, | m − = 443 |
| | pyrazole-3-carboxamide | J=7.5Hz, 1H), 7.11(m, 1 | |
| | 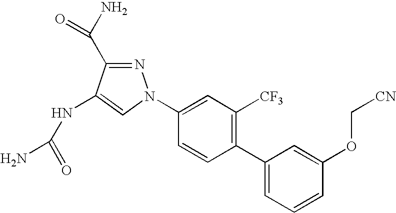 | H), 7.50(m, 1H), 7.57(s, 1 H), 7.99(s, 1H), 8.22(d, J=7.5Hz, 1H), 8.37(s, 1 H), 8.70(s, 1H), 8.73(s, 1 H) | |
| 238 | 4-[(aminocarbonyl)amino]-1-[2′-(cyanomethoxy)- | (300 MHz, DMSO-d₆): δ | m + = 445 |
| | 2-(trifluoromethyl)-1,1′-biphenyl-4-yl]-1H- | 5.11(s, 2H), 6.53(s, 2H), | m − = 443 |
| | pyrazole-3-carboxamide | 7.15(m, 1H), 7.22(m, 2 | |
| | 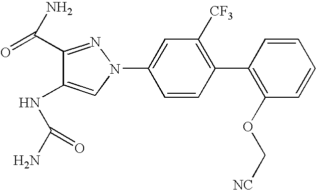 | H), 7.47(m, 2H), 7.56(s, 1 H), 7.97(s, 1H), 8.18(d, J=7.8Hz, 1H), 8.33(s, 1 H), 8.69(s, 1H), 8.71(s, 1H) | |

### Example 239

### 1-(3′-hydroxy-2-methyl-biphenyl-4-yl)-4-ureido-1H-pyrazole-3-carboxylic acid amide

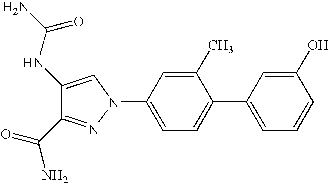

A slurry of 1-(4-iodo-3-methyl-phenyl)-4-ureido-1H-pyrazole-3-carboxylic acid amide (Example 8, 385 mg, 1.0 mmol), 3-hydroxyphenylboronic acid (276 mg, 2.0 mmol)) and (PPh₃)₄Pd (173 mg, 0.15 mmol) in anhydrous DMF (8 mL) was stirred at rt and degassed with N₂ for 10 min. A solution of Cs₂CO₃ (977 ng, 3.0 mmol) in water (2 mL) was added and degassing continued for an additional 10 min. The reaction mixture was sealed under N₂, heated to 800C for 7 h, cooled to rt and diluted with water. The resulting precipitate was collected, washed with water and air-dried. The residue was adsorbed onto silica gel (CH₃OH/acetone) and purified by column chromatography on silica gel (Biotage i40S; elution with 10% MeOH/CHCl₃) to give the title compound. ¹H NMR (300 MHz, DMSO-d₆): δ 9.48 (d, J=6.37 Hz, 1H), 8.66 (d, J=5.71 Hz, 1H), 8.56 (d, J=6.37 Hz, 1H), 7.80-7.69 (m, 3H), 7.49 (brs, 1H), 7.27-7.17 (m, 2H), 6.76-6.69 (m, 3H), 6.49 (brs, 2H), 2.27 (s, 3H). MS (ESI−) for C₈H₁₇N₅O₃ m/z 350.1 (M−H)⁻.

### Example 240

### 1-(2′-Hydroxy-2-methyl-biphenyl-4-yl)-4-ureido-1H-pyrazole-3-carboxylic acid amide

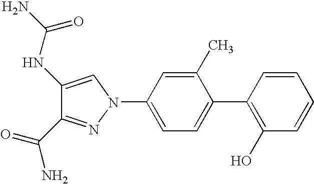

A slurry of 1-(4-iodo-3-methyl-phenyl)-4-ureido-1H-pyrazole-3-carboxylic acid amide (Example 8, 385 mg, 1.0 mmol), 2-hydroxyphenylboronic acid (276 mg, 2.0 mmol)) and (PPh₃)₄Pd (173 mg, 0.15 mmol) in anhydrous DMF (8 mL) was stirred at rt and degassed with N₂ for 10 min. A solution of Cs₂CO₃ (977 ng, 3.0 mmol) in water (2 mL) was added and degassing continued for an additional 10 min. The reaction mixture was sealed under N₂, heated to 80° C. for 7 h, cooled to rt and diluted with water. The resulting precipitate was collected, washed with water and air-dried. The residue was adsorbed onto silica gel (CH₃OH/acetone) and purified by column chromatography on silica gel (Biotage i40S; elution gradient from 5% to 20% MeOH/CHCl₃) to give the desired product. ¹H NMR (300 MHz, DMSO-d₆): δ 9.43 (s, 1H), 8.68 (s, 1H), 8.56 (s, 1H), 7.76-7.51 (m, 4H), 7.20-7.15 (m, 2H); 7.05-7.03 (m, 1H), 6.92-6.82 (m, 2H), 6.52 (brs, 2H), 2.17 (s, 3H). MS (ESI−) for C₁₈H₁₇N₅O₃ m/z350.1 (M−H)⁻.

### Example 241

### 1-(4′-Fluoro-2′-hydroxy-2-methyl-biphenyl-4-yl)-4-ureido-1H-pyrazole-3-carboxylic acid amide

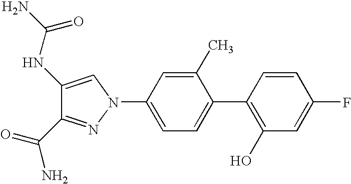

A slurry of 1-(4-iodo-3-methyl-phenyl)-4-ureido-1H-pyrazole-3-carboxylic acid amide (Example 8, 385 mg, 1.0 mmol), 2-hydroxy-4-fluorophenylboronic acid (155 mg, 1.0 mmol)) and (PPh₃)₄Pd (173 mg, 0.15 mmol) in anhydrous DMF (8 mL) was stirred at rt and degassed with N₂ for 10 min. A solution of Cs₂CO₃ (977 ng, 3.0 mmol) in water (2 mL) was added and degassing continued for an additional 10 min. The reaction mixture was sealed under N₂, heated to 80° C. for 14 h, cooled to rt and diluted with water. The resulting precipitate was collected, washed with water and air-dried. The residue was purified by column chromatography on silica gel (Biotage i40S; elution with 5% MeOH/CHCl₃) to give the desired product. ¹H NMR (300 MHz, DMSO-d₆): δ 9.95 (s, 1H), 8.67 (s, 1H), 8.55 (s, 1H), 7.76 (s, 2H), 7.67 (d, J=7.79 Hz, 1H), 7.49 (s, 1H), 7.18 (d, J=8.39 Hz, 1H), 7.09-7.05 (m, 1H), 6.70-6.65 (m, 2H), 6.50 (s, 2H), 2.15 (s, 3H). MS (ESI−) for C₁₈H₁₆FN₅O₃ m/z 368.0 (M−H)⁻.

### Example 242

### 1-(5′-Fluoro-2′-hydroxy-2-methyl-biphenyl-4-yl)-4-ureido-1H-pyrazole-3-carboxylic acid amide

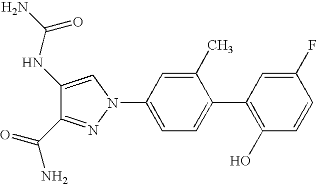

A slurry of 1-(4-iodo-3-methyl-phenyl)-4-ureido-1H-pyrazole-3-carboxylic acid amide (Example 8, 385 mg, 1.0 mmol), 2-hydroxy-5-fluorophenylboronic (155 mg, 1.0 mmol) and (PPh₃)₄Pd (173 mg, 0.15 mmol) in anhydrous DMF (8 mL) was stirred at rt and degassed with N₂ for 10 min. A solution of Cs₂CO₃ (977 ng, 3.0 mmol) in water (2 mL) was added and degassing continued for an additional 10 min. The reaction mixture was sealed under N₂, heated to 80° C. for 14 h, cooled to rt and diluted with water. The resulting precipitate was collected, washed with water and air-dried. The residue was purified by column chromatography on silica gel (Biotage i40S; elution with 4% CH₃OH/CHCl₃) to give the title compound. ¹H NMR (300 MHz, DMSO-d₆): δ 9.45 (s, 1H), 8.68 (s, 1H), 8.57 (s, 1H), 7.78-7.50 (m, 4H), 7.20 (d, J=8.20 Hz, 1H), 7.04-6.86 (m, 3H), 6.52 (s, 2H), 2.19 (s, 3H). MS (ESI−) for C₁₈H₁₆FN₅O₃ m/z 368.0 (M−H)⁻.

### Example 243

### 1-(3′-Cyanomethoxy-2-methyl-biphenyl-4-yl)-4-ureido-1H-pyrazole-3-carboxylic acid amide

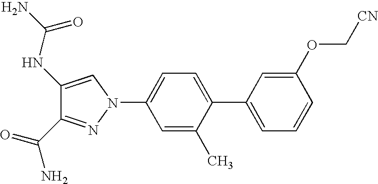

A slurry of 1-(3′-hydroxy-2-methyl-biphenyl-4-yl)-4-ureido-1H-pyrazole-3-carboxylic acid amide (Example 239, 82 mg, 0.23 mmol), bromoacetonitile (84 mg, 0.70 mmol) and potassium carbonate (65 mg, 0.47 mmol) in dry DMF (5 mL) was stirred under N₂ for 18 h. The reaction was concentrated in vacuo, the residue was re-suspended in water and the resulting precipitate collected via filtration. The solid was triturated with ether and dichloromethane. Chromatography on silica (Biotage i40S, elution with 3% MeOH/CHCl₃) was followed by trituration with ether to the title compound as a white solid. ¹H NMR (300 MHz, DMSO-d₆): δ 8.68 (s, 1H), 8.59 (s, 1H), 7.86-7.76 (m, 3H), 7.52 (s, 1H), 7.43 (t, J=7.19 Hz, 1H), 7.33 (d, J=8.39 Hz, 1H), 7.08-7.04 (m, 3H), 6.51 (s, 2H), 5.21 (s, 2H), 2.32 (s, 3H). MS (ESI−) for C₂₀H₁₈N₆O₃ m/z 389.1 (M−H).

### Example 244

### 1-(2′-Cyanomethoxy-4′-fluoro-2-methyl-biphenyl-4-yl)-4-ureido-1H-pyrazole-3-carboxylic acid amide

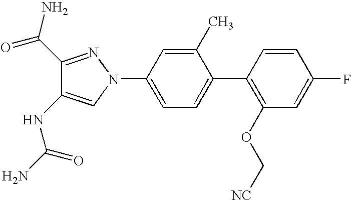

A slurry of 1-(4′-fluoro-2′-hydroxy-2-methyl-biphenyl-4-yl)-4-ureido-1H-pyrazole-3-carboxylic acid amide (Example 241, 61 mg, 0.17 mmol), bromoacetonitile (62 mg, 0.52 mmol) and potassium carbonate (48 mg, 0.35 mmol) in dry DMF (5 mL) was stirred under N₂ for 18 h. The reaction was concentrated in vacuo, the residue was re-suspended in water and the resulting precipitate collected via filtration. The precipitate was triturated with dichloromethane to the title compound as an off-white solid. ¹H NMR (300 MHz, DMSO-d₆): δ 8.67 (s, 1H), 8.58 (s, 1H), 7.81-7.71 (m, 3H), 7.52 (s, 1H), 7.31-7.19 (m, 3H), 6.99 (t, J=8.39 Hz, 1H), 6.51 (s, 2H), 5.18 (s, 2H), 2.18 (s, 3H). MS (ESI−) for C₂₀H₁₇FN₆O₃ m/z 407.0 (M−H)⁻.

### Example 245

### 1-(4-Iodo-3-methyl-phenyl)-4-(3-methyl-ureido)-1H-pyrazole-3-carboxylic acid amide

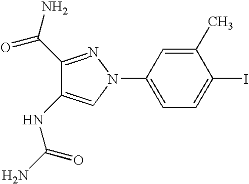

A slurry of 4-amino-1-(4-iodo-3-methyl-phenyl)-1H-pyrazole-3-carboxylic acid amide (Example 8,111 mg, 0.32 mmol) and p-nitrophenylchloroformate (79 mg, 0.39 mmol) in pyridine (2 mL) was stirred under N₂ at rt for 4 h. The thick white slurry was filtered and the solids washed with water and air dried to give [3-carbamoyl-1-(4-iodo-3-methyl-phenyl)-1H-pyrazol-4-yl]-carbamic acid 4-nitro-phenyl ester. A slurry of this ester (59 mg, 0.10 mmol) and methylamine (2 mL, 2.0M in THF, 4 mmol) was stirred under N₂ at rt for 5 days. The reaction mixture was diluted with water and filterd. The solids were washed with additional water and air dried to give the title compound. ¹H NMR (300 MHz, DMSO-d₆): δ 8.66 (s, 1H), 8.59 (s, 1H), 7.95-7.85 (m, 2H), 7.80 (s, 1H), 7.51-7.45 (m, 2H), 7.22 (s, 1H), 2.61 (d, J=3.9 Hz, 3H), 2.41 (s, 3H). MS (ESI−) for C₁₃H₁₄IN₅O₂ m/z 398.0 (M−H)⁻.

### Example 246

### 1-(5-chloro-2-fluoro-phenyl)-4-ureido-1H-pyrazole-3-carboxylic acid amide

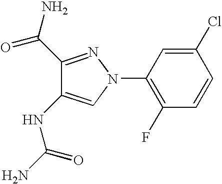

### Step 1: Preparation of 2-[(5-chloro-2-fluoro-phenyl)-hydrazono]-2-cyano-acetamide

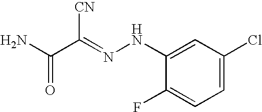

A well stirred slurry of 5-chloro-2-fluoroaniline (13.4 g, 89 mmol) and concentrated HCl (22 mL) in water (80 mL) was cooled to 5° C. and a cold solution of sodium nitrite (6.1 g, 89 mmol) in water (20 mL) was added slowly over a period of 30 min, maintaining the reaction temperature at 5° C. A cold solution of sodium acetate trihydrate (36 g, 266 mmol) in water (100 mL) was then added dropwise over 15-30 min, again maintaining the reaction temperature at 5° C. This viscous, yellow mixture was then added over a period of 30 min to a cold (5° C.) solution of sodium cyanoacetamide [previously prepared by the dissolution of cyanoacetamide (22 g, 266 mmol) in cold (5° C.) water (150 mL)/ethanol (100 mL) followed by the addition of a cold solution of sodium acetate trihydrate (12.1 g, 89 mmol) in water (30 mL)]. A thick orange slurry was obtained which was stirred for 3 h in a wet ice bath. The slurry was filtered and the solids washed with water (200 mL). This material was air-dried overnight to give an orange solid. This material was washed repeatedly with dichloromethane and the remaining yellow solid collected to provide the title compound. MS (ESI−) for C₉H₆ClFN₄O m/z 239.1 (M−H)⁻.

### Step 2: Preparation of 4-amino-5-carbamoyl-2-(5-chloro-2-fluoro-phenyl)-2H-pyrazole-3-carboxylic acid ethyl ester

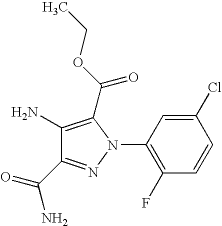

A slurry of the acetamide from Step 1 (2.04 g, 8.48 mmol) and ethyl bromoacetate (2.12 g, 12.7 mmol) was heated to 90° C. under N₂ for 5 h. The solution was cooled and poured into ice. The aqueous mixture was then stirred at rt for 3 h and filtered. The precipitate was washed with water and dried in vacuo to give the title compound as a reddish-brown solid. MS (ESI+) for C₁₃H₁₂ClFN₄O m/z 328.1 (M+H)⁺.

### Step 3: Preparation of 4-amino-5-carbamoyl-2-(5-chloro-2-fluoro-phenyl)-2H-pyrazole-3-carboxylic acid, potassium salt

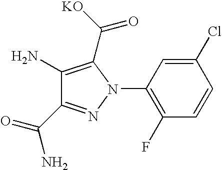

To a well stirred slurry of the ester from Step 2 (1.0 g, 3.1 mmol) in ethanol (10 mL) was added solid potassium hydroxide (1.0 g, 18 mmol) and the solution stirred at rt overnight. The reaction mixture was cooled to 5° C., filtered and the solids washed with cold ethanol and air dried to give the title compound as a light brown solid.

### Step 4: Preparation of

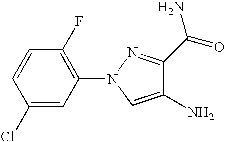

A well stirred slurry of the potassium salt from Step 3 (650 mg, 2.0 mmol) in phosphoric acid (5 mL) was slowly heated to 45° C. The mixture was held at that temperature for 2 h. The temperature was increased to 65° C. until gas evolution ceased (3 h). The solution was diluted with water (10 mL), neutralized (pH 7) with 50% sodium hydroxide solution, and the precipitate collected via filtration. The solids were washed with 5% sodium carbonate solution and air-dried to give the title compound as a light yellow solid. MS (ESI+) for C₁₀H₈ClFN₄O m/z 256.0 (M+H)⁺.

### Step 5: Preparation of 1-(5-chloro-2-fluoro-phenyl)-4-ureido-1H-pyrazole-3-carboxylic acid amide

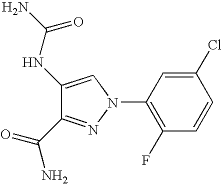

To a slurry of the amide from Step 4 (260 mg, 1.0 mmol) and potassium cyanate (99 mg, 1.2 mmol) in a solvent mixture of isopropanol/water (14 mL, 1:1) was added 75% acetic acid (7 mL) and the reaction mixture stirred at rt for 18 h. The mixture was diluted with water (30 mL) and filtered. The precipitate was washed with water and dried in vacuo to give the title compound as a light yellow solid. ¹H NMR (300 MHz, DMSO-d₆): δ 8.65 (s, 1H), 8.47 (s, 1H), 8.09 (d, J=6.81 Hz, 1H), 7.94 (s, 1H), 7.56-7.46 (m, 3H), 6.51 (s, 2H). MS (ESI−) for C₁₁H₉ClFN₅O₂ m/z296.1 (M−H)⁻.

### Example 247

### 4-[(aminocarbonyl)amino]-1-(3-fluoro-3′-hydroxy-1,1′-biphenyl-4-yl)-1H-pyrazole-3-carboxamide

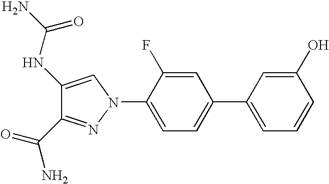

A 113 mg quantity (0.29 mmol) of Example 210 was combined in a screw-capped vial with 3-hydroxyboronic acid (0.083 g, 0.6 mmol). Cs₂CO₃ (0.295 g, 0.91 mmol) was weighed out and dissolved in 350 uL water and purged with N₂. The starting material and boronic acid were dissolved in 2.5 mL DMF, and N₂ was bubbled slowly through the solution. During the bubbling, the Cs₂CO₃ solution was added, then tetrakistriphenylphosphine palladium (0) (23 mg) was added and the vial was sealed. The reaction was heated to 83° C. overnight. The reaction was diluted with water and brought to neutrality with a few drops of 6N HCl. The product was filtered and dried under high vacuum, then triturated with CH₃OH to give the title compound. ¹H NMR (300 MHz, DMSO-d₆): δ 6.50 (bs, 2H); 6.79 (m, 1H); 7.08-7.40 (m, 4H); 7.50-7.86 (m, 4H); 7.98 (m, 1H); 8.45 (s, 1H); 8.67 (s, 1H). LCMS (ESI−) for C₁₇H₁₄F₁N₅O₃ m/z 354.2 (M−H)⁻.

### Example 248

### 4′-{3-(aminocarbonyl)-4-[(aminocarbonyl)amino]-1H-pyrazol-1-yl}-3,3′-difluoro-1,1′-biphenyl-4-carboxylic acid

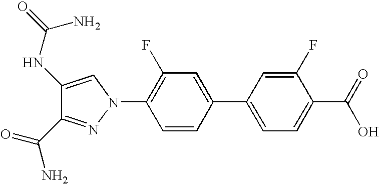

A 150 mg quantity (0.39 mmol) of Example 210 was combined in a screw-capped vial with (4-carboxy-3-fluorophenyl)boronic acid (0.1408 g, 0.771 mmol). Cs₂CO₃ (0.421 g, 1.29 mmol) was weighed out and dissolved in 0.5 mL of water and purged with N₂. The starting material and boronic acid were dissolved in 3.4 mL DMF, and N₂ was bubbled slowly through the solution. During the bubbling, the Cs₂CO₃ solution was added, then tetrakistriphenylphosphine palladium (0) (30.1 mg) was added and the vial was sealed. The reaction was heated to 83° C. overnight. The reaction was diluted with water and brought to neutrality with a few drops of 6N HCl. It was then filtered and triturated with CH₃OH. The product was purified by recrystallization from CH₃OH—CH₂Cl₂ to give the title compound. ¹H NMR (300 MHz, DMSO-d₆): δ 6.53 (bs, 2H); 7.55 (s, 1H); 7.72-8.14 (m, 8H); 8.53 (s, 1H); 8.67 (s, 1H). LCMS (ESI−) for C₁₈H₁₄F₂N₅O₄ m/z 400.1 (M−H)⁻.

### Example 249

### 4-[(aminocarbonyl)amino]-1-(3-fluoro-2′-hydroxy-1,1′-biphenyl-4-yl)-1H-pyrazole-3-carboxamide

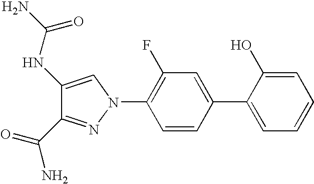

A 330 mg quantity (0.85 mmol) of Example 210 was combined in a screw-capped vial with (2-hydroxyphenyl)boronic acid (0.221 g, 0.1.6 mmol). Cs₂CO₃ (0.9104 g, 2.79 mmol) was weighed out and dissolved in 1 mL of water and purged with N₂. The starting material and boronic acid were dissolved in 6 mL DMF, and N₂ was bubbled slowly through the solution. During the bubbling, the Cs₂CO₃ solution was added, then tetrakistriphenylphosphine palladium (0) (60 mg) was added and the vial was sealed. The reaction was heated to 83° C. overnight. The reaction mixture was diluted with water and brought to neutrality with a few drops of 6N HCl. The reaction was filtered, dissolved in CH₃OH, and refiltered. The product was then recrystallized from CH₃OH-toluene and DMF-water, and triturated with CH₃OH—CH₂Cl₂. The remainder of material was purified by reverse phase chromatography on a C-8 column in DMF-water mixtures. ¹H NMR (300 MHz, DMSO-d₆): δ 6.68 (bs, 2H); 7.10 (m, 2H); 7.38 (m, 1H); 7.55 (m, 1H); 7.71 (m, 2H); 7.83 (m, 1H); 7.96 (S, 1H); 8.10 (m, 1H); 8.62 (s, 1H); 8.85 (s, 1H). LCMS (ESI−) for C₁₇H₁₄F₁N₅O₃ m/z 354.1 (M−H)⁻.

### Example 250

### 4-[(aminocarbonyl)amino]-1-[2′-(cyanomethoxy)-3-fluoro-1,1′-biphenyl-4-yl]-1H-pyrazole-3-carboxamide

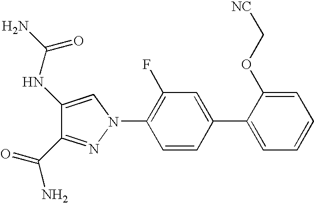

The starting material phenol (Example 249, 90 mg, 0.3 mmol) was treated with 2 mL dry DMF, ground potassium carbonate (73 mg, 0.53 mmol), and bromoacetonitrile (50 μL, 0.75 mmol). The reaction was stirred at rt overnight, then evaporated under high vacuum, taken up in water, and filtered. The product was suspended in CH₃OH and filtered. ¹H NMR (300 MHz, DMSO-d₆): δ 5.20 (s, 2H); 6.52 (bs, 2H); 7.19 (m, 1H); 7.28 (m, 1H); 7.50 (m, 5H); 7.81 (bs, 1H); 7.96 (m, 1H); 8.47 (s, 1H); 8.69 (s, 1H). LCMS (ESI+) for C₁₉H₁₅F₁N₆O₃ m/z 395.0 (M+H)⁺.

### Example 251

### 4-[(aminocarbonyl)amino]-1-(2-chloro-4-iodophenyl)-1H-pyrazole-3-carboxamide

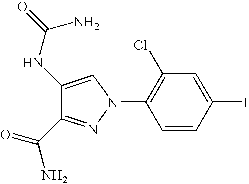

### Step 1: Preparation of 2-[(2-chloro-4-iodophenyl)hydrazono]-2-cyanoacetamide

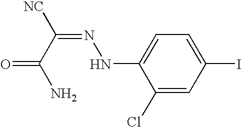

A suspension of 2-chloro-4-iodoaniline (16 g, 63 mmol) in conc. HCl (16 mL) and distilled water (50 mL) was cooled in an ice bath to 0° C. To this suspension was added a solution of sodium nitrite (4.355 g, 63 mmol) in 14 mL water over 5 min. The solution was stirred cold for 20 min. An ice-cold solution of sodium acetate trihydrate (26.048 g, 0.191 mol) in 75 mL water was added to the diazonium salt solution, and the solution was stirred a few min. In another flask, 2-cyanoacetamide (9.83 g, 0.117 mol) in 40 mL 95% ethanol and 60 ml water was cooled in an ice/salt-water bath and treated with an ice-cold solution of sodium acetate trihydrate (8.74 g, 64 mmol) in 20 mL water. The diazonium salt suspension was added to the cold cyanoacetamide with an ice-jacketed dropping funnel, over about 13 min with fast stirring. The reaction mixture was stirred cold for 15 min. The reaction was filtered on a large Buchner funnel and vacuum dried to give a brick red solid in chunks. The solid was evaporated 5 days in the vacuum oven, and purified by HPLC to yield the title compound. ¹H NMR (300 MHz, DMSO-d₆): δ 7.40 (m, 2H); 7.53 (m, 1H); 7.64 (m, 1H); 7.77 (bs, 1H). LCMS (ESI+) for C₉H₆Cl₁I₁N₄O₁ m/z 348.9 (m+H)⁺.

### Step 2: Preparation of 2-amino-3-(aminocarbonyl)-5-(2-chloro-4-iodophenyl)cyclopenta-1,3-diene-1-carboxylic acid

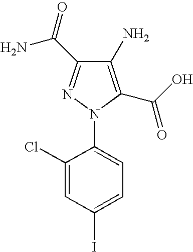

A pressure bottle was charged with pulverized 2-[(2-chloro-4-iodophenyl)hydrazono]-2-cyanoacetamide from Step 1 (19.858g, 57 mmol), DMF (50 mL), ground potassium carbonate (16 g, 116 mM), and 10.0 mL ethyl bromoacetate (85.5 mM). The bottle was flushed with N₂ and heated to 90° C. for 2 h. The crude product was saponified by adding a solution of KOH (7.63 g) in 95% ethanol (50 ml). The reaction was warmed (43° C.) for 40 min then allowed to stir at rt overnight. It was diluted with water and filtered. The filtrate was then cooled in ice and acidified with 6 N HCl. The product was obtained by filtration and analyzed by HPLC.

The red solid filtered out from the basic solution was analyzed by HPLC, and appeared to contain considerable unhydrolyzed ester. It was re-treated with KOH (9.92 g, 177 mmol) in 95% ethanol (150 mL) at 47° C. for 1 h, then stirred at rt overnight. The reaction mixture was diluted with water and filtered to give a dark red material. The pH was then adjusted to approx. 8 and the solution was filtered, giving a reddish solid. The filtrate was then acidified further to pH 1-2, giving the product as a yellow solid. The product was recrystallized (twice) by dissolving in MeOH, adding a little water, and evaporating until crystallization started.

### Step 3: Preparation of 5-amino-3-(2-chloro-4-iodophenyl)cyclopenta-1,4-diene-1-carboxamide

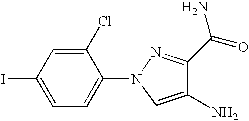

2-amino-3-(aminocarbonyl)-5-(2-chloro-4-iodophenyl)cyclopenta-1,3-diene-1-carboxylic acid from Step 2 (1.6746 g, 4.1 mmol) was treated with 8.0 mL of 85% phosphoric acid. The reaction was heated to 80° C. for 100 min, then cooled and neutralized with 2N NaOH. The reaction was filtered to give a light yellow solid, which was then triturated with CH₃OH. The total sample was treated with acetic acid and filtered. The solid was washed with water, and then washed with CH₃OH to give the title compound. ¹H NMR (300 MHz, DMSO-d₆): δ 7.19 (s, 1H); 7.37 (m, 2H); 7.45 (s, 1H); 7.83 (m, 1H); 8.04 (m, 1H). LCMS (ESI+) for C₁₀H₈C₁I₁N₄O₁ m/z 363.9 (M+2H)⁺.

### Step 4: Preparation of 4-[(aminocarbonyl)amino]-1-(2-chloro-4-iodophenyl)-1H-pyrazole-3-carboxamide

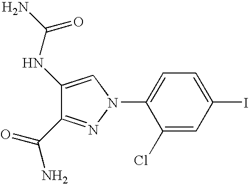

A 50-mL round bottom flask was charged with 2 mL water, 3 mL acetic acid and 3 mL isopropanol. To this mixture was added sodium cyanate (38 mg, 0.586 mmol) followed by 5-amino-3-(2-chloro-4-iodophenyl)cyclopenta-1,4-diene-1-carboxamide from Step 3 (146 mg, 0.403 mM). The reaction was stirred at rt overnight. Upon addition of water, the product crystallized. The solid was filtered and washed with water, triturated with CH₃OH—CH₂Cl₂, and then dissolved partially in DMF and water added. The final purification was done by dissolving in DMSO and adding CH₂Cl₂ to yield crystals of the title compound. ¹H NMR (300 MHz, DMSO-d₆): δ 6.48 (bs, 1H); 7.43 (m, 2H); 7.70 (bs, 1H); 7.85 (m, 1H); 8.07 (s, 1H); 8.27 (m, 1H); 8.67 (s, 1H). LCMS (ESI−) for C₁₁H₉Cl₁I₁N₀O₂ m/z 403.8 (M−H)⁻.

### Examples 252-257

### Alkylation of Phenols

A mixture of phenol (0.03 mmol) prepared according to Examples 21-141, K₂CO₃ (0.062 g, 0.045 mmol), KI (0.003 g, 0.02 mmol), and 0.045 mmol of an appropriate chloromethyl-derivative (esters or amides of chloroacetic acid, 2-chloromethyloxazole, or chloroacetone) in DMF (2 mL) was stirred at rt for 24-48 h. Solvents were removed, and water (30 mL) was added. The suspension was sonicated for 30 min and triturated for 3 h to give, after drying, the desired product.

| | | | MS |
|---|---|---|---|
| Example | Name and Structure | ¹H NMR | (M + 1) |
| 252 | 4-[(aminocarbonyl)amino]-1-[2′-(2-amino-2- | DMSO-d₆: δ 4.49(s, 2H), 6.51 | 413 |
| | oxoethoxy)-5′-fluoro-1,1′-biphenyl-4-yl]-1H- | (br s, 2H), 6.99-7.18(m, 1H), | |
| | pyrazole-3-carboxamide | 7.12-7.30(m, 3H), 7.48(br s, | |
| | 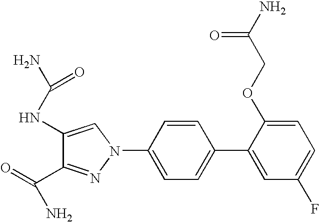 | 1H), 7.52(br s, 1H), 7.75(d, 2H, J=8.7 Hz), 7.61(br s, 1H), 7.94(d, 2H, J=8.7Hz), 8.61(s, 1H), 8.69(s, 1H) | |
| 253 | 4-[(aminocarbonyl)amino]-1-[3′-(2- | DMSO-d₆: δ 2.19(s, 3H), 4.91 | 394 |
| | oxopropoxy)-1,1′-biphenyl-4-yl]-1H-pyrazole- | (s, 2H), 6.53(br s, 2H), 6.77- | |
| | 3-carboxamide | 6.93(m, 1H), 7.20-7.44(m, | |
| | 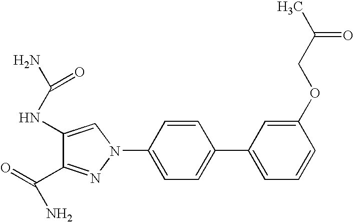 | 3H), 7.53(br s, 1H), 7.65-7.80 (m, 3H), 7.95(d, 2H, J=8.4Hz), 8.63(s, 1H), 8.69 (s, 1H) | |
| 254 | 4-[(aminocarbonyl)amino]-1-[3′-(1,3-oxazol-2- | DMSO-d₆: δ 5.35(s, 2H), 6.53 | 419 |
| | ylmethoxy)-1,1′-biphenyl-4-yl]-1H-pyrazole-3- | (br s, 2H), 7.05(d, 1H, J=7.3 | |
| | carboxamide | Hz), 7.29(s, 1H), 7.30-7.42 | |
| | 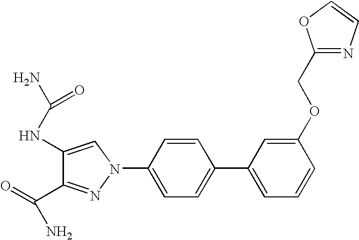 | (m, 3H), 7.53(br s, 1H), 7.75-7.88(m, 3H), 7.96(d, 2H, J=8.7Hz), 7.99(s, 1H), 8.62 (s, 1H), 8.69(s, 1H) | |
| 255 | 4-[(aminocarbonyl)amino]-1-[5′-fluoro-2′-(1,3- | CD₃OD: δ 5.13(s, 2H), 7.02- | |
| | oxazol-2-ylmethoxy)-1,1′-biphenyl-4-yl]-1H- | 7.25(m, 4H), 7.66(d, 2H, | |
| | pyrazole-3-carboxamide | J=8.7 Hz), 7.85(d, 2H, J=8.7 | |
| | 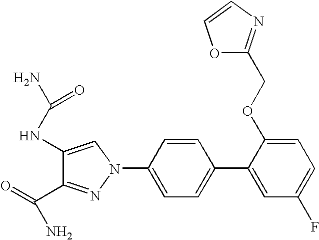 | Hz), 7.90(d, 1H, J=0.7 Hz), 8.55(s, 1H) | |
| 256 | 4-[(aminocarbonyl)amino]-1-{5′-fluoro-2′-[2- | DMSO-d₆: δ 2.63(d, 3H, | 427 |
| | (methylamino)-2-oxoethoxy]-1,1′-biphenyl-4- | J=4.5 Hz), 4.48(s, 2H), 6.52 | |
| | yl}-1H-pyrazole-3-carboxamide | (br s, 2H), 6.98-7.07(m, 1H), | |
| | 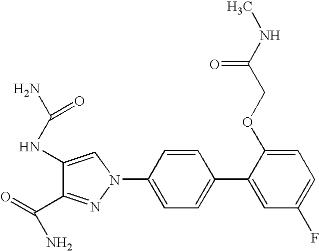 | 7.12-7.23(m, 1H), 7.26(dd, 1H, J=6.2, 3.1Hz), 7.53(br s, 1H), 7.72-7.88(m, 3H), 7.95 (d, 2H, J=8.8Hz), 8.60(s, 1H), 8.69(s, 1H) | |
| 257 | 4-[(aminocarbonyl)amino]-1-{5′-fluoro-2′-[2- | DMSO-d₆: δ 0.99(t, 3H, J=7.2 | 441 |
| | (ethylamino)-2-oxoethoxy]-1,1′-biphenyl-4-yl}- | Hz), 3.10(quintet, 2H, J=7.1 | |
| | 1H-pyrazole-3-carboxamide | Hz), 4.46(s, 2H), 6.51(br s, | |
| | 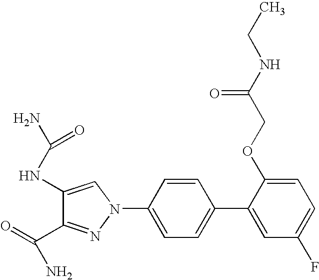 | 2H), 6.98-7.08(m, 1H), 7.12-7.22(m, 1H), 7.26(dd, 1H, J=6.2, 3.1Hz), 7.53(br s, 1H), 7.70-7.87(m, 3H), 7.95 (d, 2H, J=8.7Hz), 8.61(s, 1H), 8.69(s, 1H) | |

### Examples 258-259

### Hydrolysis of t-Butyl Esters

Concentrated HCl (3 mL) was added dropwise at rt over 3 min to 0.1 g of tert-butyl [(4′-{3-(aminocarbonyl)-4-[(aminocarbonyl)amino]-1H-pyrazol-1-yl}-1,1′-biphenyl-3-yl)oxy]acetate (Example 174) or tert-butyl [(4′-{3-(aminocarbonyl)-4-[(aminocarbonyl)amino]-1H-pyrazol-1-yl}-5-fluoro-1,1′-biphenyl-2-yl)oxy]acetate (Example 182). The suspension was stirred for 15 min, filtered, and washed with 10 mL of CHCl₃ to give, after drying under reduced pressure, the desired products.

| | | | MS |
|---|---|---|---|
| Example | Name and Structure | ¹H NMR (DMSO-d₆) | (M + 1) |
| 258 | [(4′-{3-(aminocarbonyl)-4- | δ 5.88(s, 2H), 6.00-6.90 | 396 |
| | [(aminocarbonyl)amino]-1H-pyrazol-1-yl}-1,1′- | (br s, 2H), 6.94(dd, 1H, | |
| | biphenyl-3-yl)oxy]acetic acid | J=8.0, 1.7Hz), 7.20-7.25 | |
| | 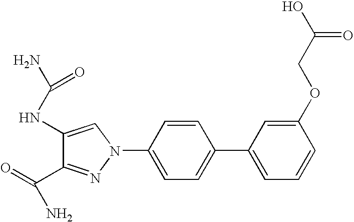 | (m, 1H), 7.28-7.42(m, 2H), 7.53(br s, 1H), 7.79 (d, 3H, J=8.7Hz), 7.95(d, 2H, J=8.7Hz), 8.51(s, 1H), 8.67(s, 1H) | |
| 259 | [(4′-{3-(aminocarbonyl)-4-[(aminocarbonyl)amino]- | δ 4.90(s, 2H), 6.53(br s, | 414 |
| | 1H-pyrazol-1-yl}-5-fluoro-1,1′-biphenyl-2- | 2H), 6.86-6.93(m, 1H), | |
| | yl)oxy]acetic acid | 7.22(br s, 1H), 7.25-7.40 | |
| | 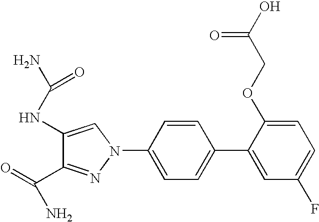 | (m, 2H), 7.53(br s, 1H), 7.75-7.85(m, 3H), 7.95 (d, 2H, J=8.4Hz), 8.62(s, 1H), 8.69(s, 1H) | |

### Example 260

### 4-[(aminocarbonyl)amino]-1-[4-(4,4,5,5-tetramethyl-1,3,2-dioxaborolan-2-yl)phenyl]-1H-pyrazole-3-carboxamide

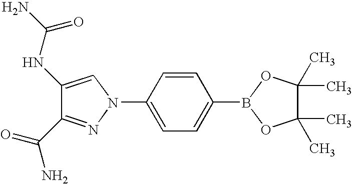

4-[(Aminocarbonyl)amino]-1-(4-iodophenyl)-1H-pyrazole-3-carboxamide (1.93 g, 5.2 mmol, prepared according to the process of Example 214), palladium acetate (0.04 g, 0.16 mmol), potassium acetate (1.53 g, 15.6 mmol), and 1.43 g (5.62 mmol) of bis(pinacolato)diboron were added to a flask and placed under vacuum, then refilled with N₂. The vacuum-N₂ cycle was repeated twice. The DMF (20 mL) was injected under N₂. (The DMF was bubbled with N₂ for 15 min before addition). The mixture was heated and stirred at 85° C. for 5 h. Most of liquids were removed under reduced pressure. The solid was triturated with water (80 mL) for 30 min., filtered, triturated with the petroleum ether (50 mL) for 1 h, filtered and dried. Most of the solid was dissolved in 1 L of the CHCl₃ and filtered. The solvent was removed under reduced pressure to give, after drying, the desired product. ¹H NMR (CDCl₃): δ 1.37 (s, 12H), 4.70 (br s, 2H), 5.46 (br s, 1H), 6.88 (br s, 1H), 7.38 (d, 2H, J=8.7 Hz), 7.90 (d, 2H, J=8.7 Hz), 8.59 (s, 1H), 8.69 (br s, 1H). ESI mass spectrum for C₁₇H₂₃BN₅O₄⁺: 372 (M+1).

### Example 261

### 4-[(aminocarbonyl)amino]-1-(2-fluoro-2′-hydroxy-1,1′-biphenyl-4-yl)-1H-pyrazole-3-carboxamide

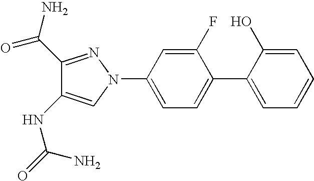

To a 2-neck flask were added a stir bar, 4-[(aminocarbonyl)amino]-1-(4-bromo-3-fluorophenyl)-1H-pyrazole-3-carboxamide (Example 225, 0.398 g, 1.2 mmol), tetrakis(triphenylphosphine)palladium(0) (0.13 g, 0.12 mmol), 2-hydroxyphenylboronic acid (0.32 g, 2.3 mmol), and dry DMF (8 mL) via syringe; this stirring solution was flushed with vacuum/N₂. Cs₂CO₃ (0.92 g, 2.8 mmol) was mixed with H₂O (1.5 mL) and N₂ was bubbled through the resultant solution for 10 min. The solution was then added by syringe to the reaction mixture above which was then immersed in an oil bath (80° C.). After stirring overnight, the heat was removed; after 5 min, the mixture was poured onto ice (200 mL), stirred 30 minutes, and filtered (4×30 mL H₂O washes); air-dried to give an off-white solid. This solid was stirred with CH₂Cl₂ (100 mL) and filtered (3×20 mL CH₂Cl₂ rinses) to give an off-white solid which was stirred with methanol (70 mL) and filtered (1×10 mL methanol rinse); airdrying and then high vacuum drying overnight of the filtered solid gave the title compound as an off-white powder. ¹H NMR (300 MHz, DMSO-d₆) δ 9.63 (s, 1H), 8.68 (s, 1H), 8.63 (s, 1H), 7.86 (m, 2H), 7.75 (m, 1H), 7.56 (s, 1H), 7.46 (m, 1H), 7.21 (m, 2H), 6.90 (m, 2H), 6.53 (brs, 2H); MS (ESI−) for C₁₇H₁₄FN₅O₃ m/z 354.0 (M−H)⁻.

### Example 262

### 4-[(aminocarbonyl)amino]-1-(2,4′-difluoro-2′-hydroxy-1,1′-biphenyl-4-yl)-1H-pyrazole-3-carboxamide

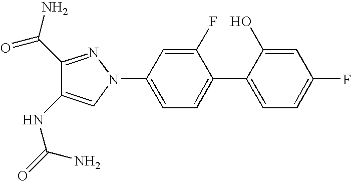

To a 2-neck flask were added a stir bar, 4-[(aminocarbonyl)amino]-1-(4-bromo-3-fluorophenyl)-1H-pyrazole-3-carboxamide (Example 225, 0.406 g, 1.2 mmol), tetrakis(triphenylphosphine)palladium(0) (0.137 g, 0.12 mmol), 2-hydroxy-4-fluorophenylboronic acid (0.370 g, 2.4 mmol), and dry DMF (8 mL) via syringe; this stirring solution was flushed with vacuum/N₂. Cs₂CO₃ (0.936 g, 2.9 mmol) was mixed with H₂O (1.5 mL) and N₂ was bubbled through the resultant solution for 10 min. The solution was then added by syringe to the reaction mixture above which was then immersed in an oil bath (80° C.). After stirring overnight, the heat bath was removed; after 10 min. the reaction mixture was poured onto ice (200 mL), stirred 30 minutes, and filtered (4×30 mL H₂O washes). The filtered solid was air-dried to give a grey solid that was transferred to a Si gel column and chromatographed (gradient from 2% to 10% DMF/toluene). Fractions containing the desired product were stripped to near dryness and then mixed with CH₂Cl₂ (5 mL), resulting in a white precipitate. This precipitate was filtered (4×5 mL CH₂Cl₂ washes) and dried (air—followed by high vacuum) to give a white solid. This white solid was stirred with CH₂Cl₂ (3×30 mL) and filtered to give the title compound as an ivory solid. ¹H NMR (300 MHz, DMSO-d₆) δ 10.18 (s, 1H), 8.68 (s, 1H), 8.63 (s, 1H), 7.87 (m, 2H), 7.75 (m, 1H), 7.55 (brs, 1H), 7.44 (m, 1H), 7.23 (m, 1H), 6.71 (m, 2H), 6.53 (brs, 2H); MS (ESI−) for C₁₇H₁₃F₂N₅O₃ m/z 372.1 (M−H)⁻.

### Example 263

### 4-[(aminocarbonyl)amino]-1-(2,5′-difluoro-2′-hydroxy-1,1′-biphenyl-4-yl)-1H-pyrazole-3-carboxamide

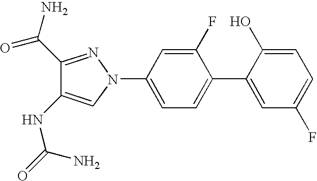

To a 2-neck flask were added a stir bar, 4-[(aminocarbonyl)amino]-1-(4-bromo-3-fluorophenyl)-1H-pyrazole-3-carboxamide (Example 225, 0.421 g, 1.2 mmol), tetrakis(triphenylphosphine)palladium(0) (0.142 g, 0.12 mmol), 2-hydroxy-5-fluorophenylboronic acid (0.384 g, 2.5 mmol), and dry DMF (8 mL) via syringe; this stirring solution was flushed with vacuum/N₂. Cs₂CO₃ (0.970 g, 3.0 mmol) was mixed with H₂O (1.5 mL) and N₂ was bubbled through the resultant solution for 10 min. The solution was then added by syringe to the reaction mixture above which was then immersed in an oil bath (80° C.). After stirring overnight, the heat bath was removed; after 10 min. the reaction mixture was poured onto ice (200 mL), stirred 30 minutes, and filtered (4×30 mL H₂O washes). The filtered solid was air-dried to give a brown solid. Trituration with CH₂Cl₂ (3×5 mL) followed by trituration with methanol (3×5 mL) gave the title compound as an off-white solid. ¹H NMR (300 MHz, DMSO-d₆) δ 9.64 (s, 1H), 8.68 (s, 1H), 8.64 (s, 1H), 7.88 (m, 2H), 7.77 (m, 1H), 7.56 (brs, 1H), 7.49 (m, 1H), 7.07 (m, 2H), 6.91 (m, 1H), 6.53 (brs, 2H); MS (ESI−) for C₁₇H₁₃F₂N₅O₃ m/z 372.0 (M−H)⁻.

### Example 264

### 4-[(aminocarbonyl)amino]-1-(2-fluoro-4′-hydroxy-1,1′-biphenyl-4-yl)-1H-pyrazole-3-carboxamide

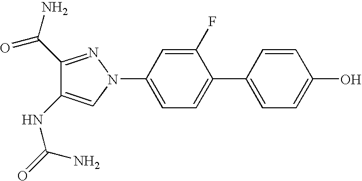

To a reaction vial were added 4-[(aminocarbonyl)amino]-1-(4-bromo-3-fluorophenyl)-1H-pyrazole-3-carboxamide (Example 225, 0.193 g, 0.56 mmol), tetrakis(triphenylphosphine)palladium(0) (0.065 g, 0.056 mmol), 4-hydroxyphenylboronic acid (0.156 g, 1.13 mmol), and dry DMF (10 mL) via syringe; this was flushed with vacuum/N₂. To the reaction mixture was added an N₂-purged aqueous solution of Cs₂CO₃ (0.6 mL, 2.2M) which was then placed in a shaker block at 80° C. After 62 h the heat was turned off and the mixture allowed to cool to room temperature overnight. The mixture was then poured onto ice (100 mL), stirred 30 min, filtered through a sintered glass funnel, and the filtered solid washed with H₂O (8×20 mL). The light brown solid was washed with methanol (3×5 mL) and air-dried to give the title compound as an off-white solid. ¹H NMR (300 MHz, DMSO-d₆) δ 9.67 (brs, 1H), 8.67 (brs, 1H), 8.63 (brs, 1H), 7.90 (m, 2H), 7.77 (m, 1H), 7.56 (m, 2H), 7.40 (m, 2H), 6.85 (m, 2H), 6.52 (brs, 2H); MS (ESI−) for C₁₇H₁₄FN₅O₃ m/z. 355.3 (M−H)⁻.

### Example 265

### 4-[(aminocarbonyl)amino]-1-(2,3′-difluoro-4′-hydroxy-1,1′-biphenyl-4-yl)-1H-pyrazole-3-carboxamide

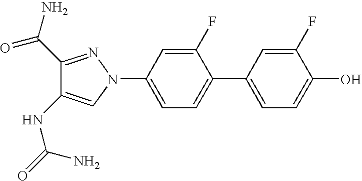

To a reaction vial were added 4-[(aminocarbonyl)amino]-1-(4-bromo-3-fluorophenyl)-1H-pyrazole-3-carboxamide (Example 225, 0.262 g, 0.77 mmol), tetrakis(triphenylphosphine)palladium(0) (0.088 g, 0.077 mmol), 3-fluoro-4-hydroxyphenylboronic acid (0.24 g, 1.5 mmol), and dry DMF (10 mL) via syringe; this was flushed with vacuum/N₂. To the reaction mixture above was added an N₂-purged aqueous solution of Cs₂CO₃ (0.84 mL, 2.2M) which was then placed in a shaker block at 80° C. After 62 h the heat was turned off and the mixture allowed to cool to room temperature overnight. The mixture was then poured onto ice (100 mL), stirred 30 min, filtered through a sintered glass funnel, and the filtered solid washed with H₂O (8×20 mL). The light brown solid was washed with THF (3×5 mL), dissolved in DMF (1.5 mL), poured into CH₂Cl₂ (10 mL), stirred 10 min, filtered through a sintered glass funnel (4×5 mL CH₂Cl₂ washes) and air-dried to give the title compound as an off-white solid. ¹H NMR (300 MHz, DMSO-d₆) δ 10.15 (brs, 1H), 8.68 (s, 1H), 8.64 (m, 1H), 7.91 (m, 2H), 7.79 (m, 1H), 7.63 (m, 1H), 7.55 (brs, 1H), 7.39 (m, 1H), 7.24 (m, 1H), 7.04 (m, 1H), 6.53 (brs, 2H); MS (ESI−) for C₁₇H₁₃F₂N₅O₃ m/z 373.3 (M−H)⁻.

### Example 266

### 4-[(aminocarbonyl)amino]-1-[2′-(2-amino-2-oxoethoxy)-2-fluoro-1,1′-biphenyl-4-yl]-1H-pyrazole-3-carboxamide

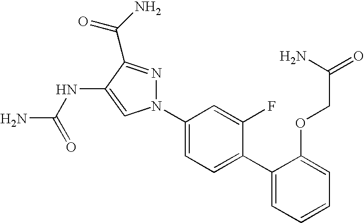

To a flask were added 4-[(aminocarbonyl)amino]-1-(2-fluoro-2′-hydroxy-1,1′-biphenyl-4-yl)-1H-pyrazole-3-carboxamide (Example 261, 0.082 g, 0.23 mmol), dry DMF (5 mL), K₂CO₃ (0.096 g, 0.69 mmol), and 2-bromoacetamide (0.064 g, 0.46 mmol); the reaction mixture was flushed with vacuum/N₂ and stirred under N₂. After stirring overnight, the mixture was stripped to a dark, oily solid which was stirred with H₂O (20 mL), filtered and the filtered brown solid washed with H₂O (5×5 mL) to give a brown solid. Trituration with a 1:1 mix of CH₂Cl₂ and methanol (4×5 mL) gave the title compound as an off white solid. ¹H NMR (300 MHz, DMSO-d₆) δ 8.71 (brs, 1H), 8.68 (m, 1H), 7.94 (m, 2H), 7.82 (m, 1H), 7.61 (m, 2H), 7.42 (m, 2H), 7.34 (m, 1H), 7.08 (m, 2H), 6.99 (brs, 1H), 6.57 (brs, 2H), 4.48 (s, 2H); MS (ESI−) for C₁₉H₁₇FN₆O₄ m/z 412.3 (M−H)⁻.

### Example 267

### 4-[(aminocarbonyl)amino]-1-(2-fluoro-2′-methoxy-1,1′-biphenyl-4-yl)-1H-pyrazole-3-carboxamide

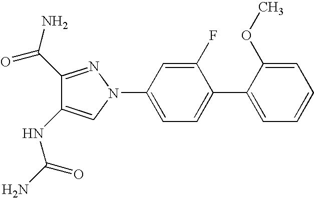

A mixture of 4-[(aminocarbonyl)amino]-1-(2-fluoro-2′-hydroxy-1,1′-biphenyl-4-yl)-1H-pyrazole-3-carboxamide (Example 261, 0.0900 g, 0.25 mmol), K₂CO₃ (0.079 g, 0.57 mmol), and DMF (5 mL) was flushed with vacuum/N₂. To this stirring mix was added CH₃I (0.037 mL, 0.59 mmol). After three nights, the mixture was stripped to dryness, stirred 1 h with H₂O (5 mL), filtered and washed with H₂O (3×5 mL), CH₂Cl₂ (3×5 mL), and methanol (3×5 mL). Air-drying 1 h gave the title compound as an off-white solid. ¹H NMR (300 MHz, DMSO-d₆) δ 8.69 (m, 1H), 8.64 (m, 1H), 7.87 (m, 2H), 7.77 (m, 1H), 7.56 (brs, 1H), 7.43 (m, 2H), 7.27 (m, 1H), 7.13 (m, 1H), 7.04 (m, 1H), 6.54 (brs, 2H)., 3.75 (s, 3H); MS (ESI−) for C₁₈H₁₆FN₅O₃ m/z 369.35 (M−H).

### Example 268

### 4-[(aminocarbonyl)amino]-1-[2′-(2-amino-2-oxoethoxy)-2,4′-difluoro-1,1′-biphenyl-4-yl]-1H-pyrazole-3-carboxamide

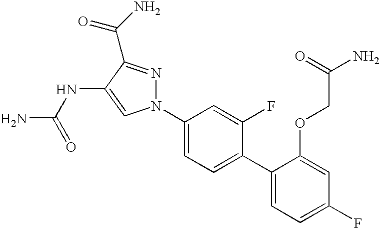

To a flask were added 4-[(aminocarbonyl)amino]-1-(2,4′-difluoro-2′-hydroxy-1,1′-biphenyl-4-yl)-1H-pyrazole-3-carboxamide (Example 262, 0.067 g, 0.2 mmol), DMF (5 mL, dry), K₂CO₃ (0.074 g, 0.5 mmol), and 2-bromoacetamide (0.049 g, 0.4 mmol); the reaction mixture was flushed with vacuum/N₂ and stirred under N₂. After 3 days, the mixture was poured onto ice (50 mL), stirred for 1 h, and then filtered (4×2 mL H₂O rinses); the filtered solid was air-dried for an hour, then placed under high vacuum overnight to give the title compound as a pale grey solid. ¹H NMR (300 MHz, DMSO-d₆) δ 8.67 (brs, 1H), 8.65 (m, 1H), 7.89 (m, 2H), 7.78 (m, 1H), 7.56 (m, 2H), 7.36 (m, 2H), 6.95 (m, 3H), 6.53 (brs, 2H), 4.50 (s, 2H); MS (ESI−) for C₁₉H₁₆F₂N₆O₄ m/z 430.36 (M−H)⁻.

### Example 269

### 4-[(aminocarbonyl)amino]-1-(2,5′-difluoro-2′-methoxy-1,1′-biphenyl-4-yl)-1H-pyrazole-3-carboxamide

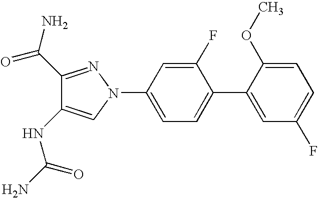

A mixture of 4-[(aminocarbonyl)amino]-1-(2,5′-difluoro-2′-hydroxy-1,1′-biphenyl-4-yl)-1H-pyrazole-3-carboxamide (Example 263, 0.088 g, 0.24 mmol), K₂CO₃ (0.079 g, 0.57 mmol), and DMF (3 mL) was flushed with vacuum/N₂. To this stirring mix was added CH₃I (0.036 mL, 0.59 mmol). After three nights, the mixture was stripped to dryness, stirred 1 h with H₂O (5 mL), filtered and washed with H₂O (3×5 mL), CH₂Cl₂ (3×5 mL), and methanol (3×5 mL). Air-drying 1 h gave the title compound as an off-white solid. ¹H NMR (300 MHz, DMSO-d₆) δ 8.67 (m, 1H), 8.64 (m, 1H), 7.90 (m, 2H), 7.78 (m, 1H), 7.56 (brs, 1H), 7.47 (m, 1H), 7.19 (m, 3H), 6.53 (brs, 2H), 3.77 (s, 3H); MS (ESI−) for C₁₈H₁₅F₂N₅O₃ m/z 387.3 (M−H)⁻.

### Example 270

### 4-[(aminocarbonyl)amino]-1-{3-chloro-4-[(2-fluorophenyl)thio]phenyl}-1H-pyrazole-3-carboxamide

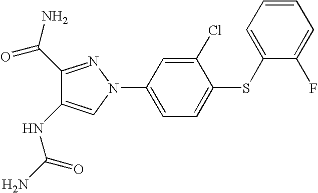

To a pressure bottle were added a stir bar, 4-[(aminocarbonyl)amino]-1-(3-chloro-4-iodophenyl)-1H-pyrazole-3-carboxamide (Example 6, 0.71 g, 1.7 mmol), CuI (0.050 g, 0.2 mmol), N,N-diisopropylethylamine (0.5 mL, 2.7 mmol), 2-propanol (150 mL), 2-fluorothiophenol (0.5 mL, 4.6 mmol), ethylene glycol (0.20 mL, 3.2 mmol). The mixture was flushed (vacuum/N₂), capped and immersed in an oil bath (105° C.). After overnight stirring, more N,N-diisopropylethylamine (1 mL, 5.4 mmol) and CuI (0.07 g, 3.6 mmol) were added. After a second night at 105° C., the mixtrure was removed from the heating bath and allowed to cool. The rt mixture was filtered (2×5 mL 2-propanol washes); the filtrate was evaporated to dryness, stirred with CHCl₃ (30 mL) and filtered to give a light brown solid. This material was transferred to a silica gel chromatography column and elutec with a gradient (0 to 5% methanol/chloroform) to give a white solid (mixture of 27% starting material (4-[(aminocarbonyl)amino]-1-(3-chloro-4-iodophenyl)-1H-pyrazole-3-carboxamide) and 63% product (4-[(aminocarbonyl)amino]-1-{3-chloro-4-[(2-fluorophenyl)thio]phenyl}-1H-pyrazole-3-carboxamide)). This mixture was used as described in the following paragraph.

The mixture of 4-[(aminocarbonyl)amino]-1-(3-chloro-4-iodophenyl)-1H-pyrazole-3-carboxamide (0.18 g, 0.44 mmol) and 4-[(aminocarbonyl)amino]-1-{3-chloro-4-[(2-fluorophenyl)thio]phenyl}-1H-pyrazole-3-carboxamide (0.315 g, 0.77 mmol) was stirred with DMF (12 mL) and flushed with vacuum/N₂. To this was added CuI (0.057 g, 0.3 mmol), N,N-diisopropylethylamine (3 mL, 17 mmol), ethylene glycol (0.1 mL, 4 mmol), and 2-fluorothiophenol (0.20 mL, 2 mmol). The stirring mixture was then immersed in an oil bath (80° C.). After four nights, the reaction was removed from heat, poured onto ice (100 mL) and Na₂CO₃ (0.7 g in 10 mL H₂O), stirred 90 min and then filtered (5×5 mL H₂O washes) to give a yellow-brown solid which was triturated with CH₂Cl₂ (4×10 mL) to give a brown liquid and a pale yellow solid. This solid was stirred with THF (20 mL), filtered (3×20 mL THF rinses), and the filtrate evaporated to dryness, triturated with methanol (4×5 mL) and air-dried to give the title compound as an ivory solid. ¹H NMR (300 MHz, DMSO-d₆) δ 8.66 (brs, 1H), 8.59 (m, 1H), 8.21 (m, 1H), 7.90 (brs, 1H), 7.82 (m, 1H), 7.55 (brs, 1H), 7.50 (m, 1H), 7.39 (m, 2H), 7.30 (m, 1H), 7.09 (d, J=9.7 Hz, 1H), 6.53 (brs, 2H); MS (ESI−) for C₁₇H₁₃ClFN₅O₂S m/z 405.83 (M−H)⁻.

### Example 271

### 4-[(aminocarbonyl)amino]-1-{3-chloro-4-[(2-fluorophenyl)sulfonyl]phenyl}-1H-pyrazole-3-carboxamide

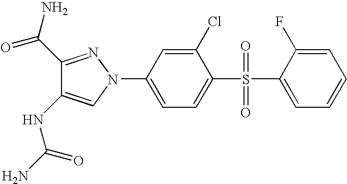

To a flask containing 4-[(aminocarbonyl)amino]-1-{3-chloro-4-[(2-fluorophenyl)thio]phenyl}-1H-pyrazole-3-carboxamide (Example 270, 0.102 g, 0.25 mmol), and MCPBA (0.212 g, 77%, 0.9 mmol) was added CH₂Cl₂ (10 mL). The mixture was stirred at room temperature. After 9 d, more MCPBA (0.14 g, 77%, 0.6 mmol) was added. After another 7 d, a solution of Na₂CO₃ (1.0 g in 10 mL H₂O) was added to the reaction mixture. After stirring 30 min, the mixture was filtered and the solid washed with H₂O (3×5 mL) and CH₂Cl₂ (3×5 mL). The solid was air-dried 60 min, and then dried under high vacuum overnight to give the title compound as a white solid. ¹H NMR (300 MHz, DMSO-d₆) δ 8.74 (m, 1H), 8.69 (brs, 1H), 8.32 (m, 2H), 8.16 (m, 2H), 8.03 (brs, 1H), 7.83 (m, 1H), 7.64 (brs, 1H), 7.52 (m, 1H), 7.41 (m, 1H), 6.58 (brs, 2H); MS (ESI−) for C₁₇H₁₃ClFN₅O₄S m/z 437.83 (M−H)⁻.

### Example 272

### 4-[(aminocarbonyl)amino]-1-(2-chloro-4′-hydroxy-1,1′-biphenyl-4-yl)-1H-pyrazole-3-carboxamide

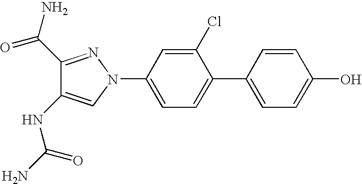

To a reaction vial were added 4-[(aminocarbonyl)amino]-1-(3-chloro-4-iodophenyl)-1H-pyrazole-3-carboxamide (Example 6, 0.255 g, 0.63 mmol), tetrakis(triphenylphosphine)palladium(0) (0.073 g, 0.063 mmol), 4-hydroxyphenylboronic acid (0.173 g, 1.26 mmol), and dry DMF (10 mL) via syringe; this was flushed with vacuum/N₂. To the reaction mixture above was added an N₂-purged aqueous solution of Cs₂CO₃ (0.7 mL, 2.2M) which was then placed in a shaker block at 80° C. After 14 h the mixture was removed from the heating block and then poured onto ice (100 mL), stirred 30 min, filtered through a sintered glass funnel, and the filtered solid washed with H₂O (8×20 mL). The brown solid was then washed with methanol/THF (1:1, 4×20 mL) and the combined filtrates mixed with silica gel, evaporated to dryness and eluted through a 70 g silica gel column utilizing a gradient from 5% to 25% DMF/toluene. Rotary evaporation of fractions gave the title compound as an ivory solid. ¹H NMR (300 MHz, DMSO-d₆) δ 9.64 (brs, 1H), 8.68 (s, 1H), 8.63 (s, 1H), 8.13 (m, 1H), 7.88 (m, 2H), 7.53 (brs, 1H), 7.44 (m, 1H), 7.28 (m, 2H), 6.83 (m, 2H), 6.52 (brs, 2H); MS (ESI−) for C₁₇H₁₄ClN₅O₃ m/z 371.78 (M−H)⁻.

### Example 273

### 1-(3-chloro-2-fluoro-phenyl)-4-ureido-1 h-pyrazole-3-carboxylic acid amide

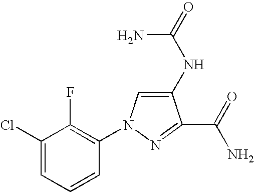

### Step 1: Preparation of 2-cyano-2-[(3-chloro-2-fluoro-phenyl)-hydrazono]-acetamide

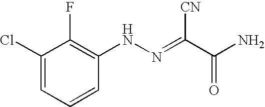

A well stirred solution of 3-chloro-2-fluoro-aniline (1.0 g, 6.9 mmol) and conc. HCl (1.7 mL) in water (40 mL) was cooled to −2° C. and a cold solution of sodium nitrite (470 mg, 6.9 mmole in 1 mL water) was added slowly over a period of 15 min, maintaining the reaction temperature between −2 and −1.5° C. A bright yellow reaction mixture resulted and was allowed to stir for 30 min. This mixture was slowly added to a cold (5° C.), rapidly stirred solution of sodium cyanoacetamide [previously prepared by the dissolution of cyanoacetamide (1.7 g, 21 mmol) in cold (5° C.) water (4 mL)/ethanol (25 mL) followed by the addition of a cold solution of sodium acetate trihydrate (0.94 g, 5 mmol) in water (5 mL)] over a period of 15 min. A bright terra cotta red slurry was obtained and stirred for 1 h. The solids were filtered, washed with water and with ether. The solids were dried in vacuo to give the title compound. MS (ESI+) for C₉H₆ClFN₄O m/z 241 (M+H)⁺.

### Step 2: Preparation of 4-amino-5-carbamoyl-2-(3-chloro-2-fluoro-phenyl)-2H-pyrazole-3-carboxylic acid ethyl ester

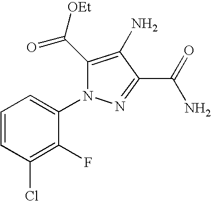

A slurry of freshly ground potassium carbonate (670 mg, 2.3 mmol), 2-cyano-2-[(3-chloro-2-fluoro-phenyl)-hydrazono]-acetamide (Step 1, 510 mg, 2.1 mmol) and ethyl bromoacetate (530 mg, 1.5 mmol) in DMF (5 mL) was heated to 100° C. with a drying tube for 4 h. The solution was cooled, and ice was added. The resulting slurry was then stirred at rt for 30 min and filtered. The precipitate was washed with water and minimal cold ethanol, which removed all color from the solid. The solids were dried in vacuo to give the title compound as an off-white solid. MS (ESI+) for C₁₃H₁₂ClFN₄O₃ m/z 328.0 (M+H)⁺.

### Step 3: Preparation of 4-amino-5-carbamoyl-2-(3-chloro-2-fluoro-phenyl)-2H-pyrazole-3-carboxylic acid potassium salt

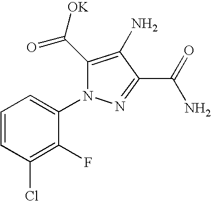

A slurry of potassium hydroxide (500 mg, 8.9 mmol) and 4-amino-5-carbamoyl-2-(3-chloro-2-fluoro-phenyl)-2H-pyrazole-3-carboxylic acid ethyl ester (Step 2, 410 mg, 1.3 mmol) in 95% ethanol (8 mL) was stirred at rt for 4 h. Additional potassium hydroxide (1.0 g, 17.8 mmol) was added and at 5 h, the temperature was increased from rt to 45° C. for one additional hour. The reaction was allowed to cool and the precipitate was collected, washed with ethanol and dried in vacuo to give the title compound.

### Step 4: Preparation of 4-Amino-1-(3-chloro-2-fluoro-phenyl)-1H-pyrazole-3-carboxylic acid amide

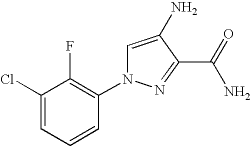

A well stirred slurry of the potassium salt from Step 3 (250 mg, 0.74 mmol) in 85% phosphoric acid (3 mL) was slowly heated to 45° C. and held at that temperature for 2 h. The reaction temperature was increased to 65° C. for an additional 2 h. The reaction mixture was then cooled to 5° C., diluted with water (15 mL), neutralized (to pH 6) with 50% sodium hydroxide solution and filtered. The resulting solid was washed with water, filtered, and dried in vacuo to give the title compound as a white solid. ¹H NMR (300 MHz, DMSO-d₆) δ 7.65-7.60 (m, 1H), 7.41-7.30 (m, 3H), 7.18-7.09 (m, 2H), 4.68 (brs, 2H).

### Step 5: Preparation of 1-(3-Chloro-2-fluoro-phenyl)-4-ureido-1H-pyrazole-3-carboxylic acid amide

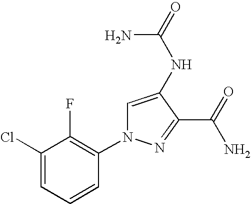

To a slurry of 4-amino-1-(3-chloro-2-fluoro-phenyl)-1H-pyrazole-3-carboxylic acid amide (Step 4,150 mg, 0.59 mmol) and potassium cyanate (57 mg, 0.71 mmol) in a solvent mixture of isopropanol/water (10 mL, 1:1) was added 75% acetic acid (5 mL) and the reaction mixture stirred at rt for 3 h. The mixture was diluted with water (10 mL) and the precipitate filtered, washed with water and dried in vacuo to give crude product as an off-white solid. This solid was triturated with CH₂Cl₂ to provide the title compound. MS (ESI−) for C₁₁H₉ClFN₅O₂ m/z 295.9 (M−H)⁻. ¹H NMR (300 MHz, DMSO-d₆) δ 8.63-8.60 (m, 1H), 8.41-8.38 (m, 1H), 7.85-7.74 (m, 2H), 7.60-7.52 (m, 2H), 7.49-7.26 (m, 1H), 6.47 (brs, 2H).

### Example 274

### 4-[(aminocarbonyl)amino]-1-(2-ethoxy-4′-hydroxy-1,1′-biphenyl-4-yl)-1H-pyrazole-3-carboxamide

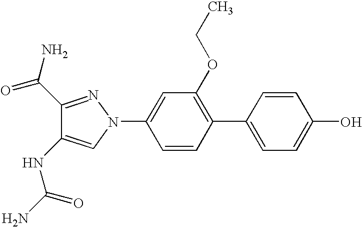

N₂ was bubbled through a mixture of 1-(4-bromo-3-ethoxyphenyl)-4-ureido-1H-pyrazole-3-carboxylic acid amide (Example 3, 486 mg, 1.32 mmol), 4-hydroxyphenylboronic acid (363 mg, 2.63 mmol) and Pd(PPh₃)₄ (228 mg, 0.198 mmol) in dry DMF(10 mL). A solution of Cs₂CO₃ (1.29 g, 3.95 mmol) in water (2.5 mL) was added and the mixture heated at 80° C. for 15 h. Upon cooling, ice-water (100 mL) was added and the precipitate filtered. Chromatography over silica gel (0-10% methanol-chloroform) afforded the title compound as a tan solid. ¹H NMR (300 MHz, DMSO-d₆) δ 1.30 (t, J=6.7 Hz, 3H), 4.14 (q, J=6.7 Hz, 2H), 6.51 (s, 2H), 6.78 (d, J=7.5 Hz, 1H), 7.34 (m, 3H), 7.46 (m, 1H), 7.56 (m, 2H), 7.85 (s, 1H), 8.59 (s, 1H), 8.67 (s, 1H), 9.47 (s, 1H); LCMS: m+=382 and m−=380.

### Example 275

### 4-[(aminocarbonyl)amino]-1-[4′-(2-amino-2-oxoethoxy)-2-ethoxy-1,1′-biphenyl-4-yl]-1H-pyrazole-3-carboxamide

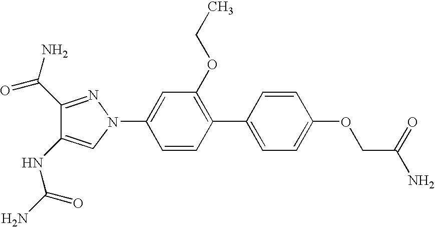

To 4-[(aminocarbonyl)amino]-1-(2-ethoxy-4′-hydroxy-1,1′-biphenyl-4-yl)-1H-pyrazole-3-carboxamide (Example 274,120 mg, 0.315 mmol) and bromoacetamide (130 mg, 0.944 mmol) was added dry DMF (4 mL) and powdered potassium carbonate (87 mg, 0.629 mmol). The mixture was stirred at 25° C. for 2 days then at 45° C. for 24 h. Upon cooling, water (60 mL) was added and the precipitate filtered. Repeated trituration with 10% CH₃OH-chloroform gave the title compound as a beige solid. ¹H NMR (300 MHz, DMSO-d₆) δ 1.30 (t, J=6.6 Hz, 3H), 4.16 (q, J=6.6 Hz, 2H), 4.44 (s, 2H), 6.51 (s, 2H), 6.97 (d, J=8.1 Hz, 2H), 7.45 (m, 8H), 7.86 (m, 1H), 8.59 (s, 1H), 8.66 (s, 1H); LCMS: m+=439, m−=437.

### Example 276

### 4-[(aminocarbonyl)amino]-1-(4-bromo-3-hydroxyphenyl)-1H-pyrazole-3-carboxamide

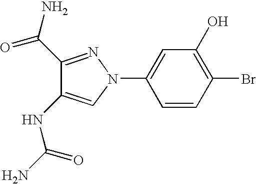

1-(4-bromo-3-ethoxyphenyl)-4-ureido-1H-pyrazole-3-carboxylic acid amide (Example 3, 368 mg, 1.00 mmol) was stirred in dry CH₂Cl₂ (5 mL) under N₂ then cooled to −78° C. A 1 M solution of boron tribromide in CH₂Cl₂ (4.00 mL, 4.00 mmol) was added and the reaction stirred at this temperature for 30 min when the cooling bath was removed. After 20 h, water (25 mL) was added and the brown solid filtered. Chromatography over silica gel (10% methanol-chloroform) afforded the product which was washed with 5 mL each of acetone, methanol, ethyl acetate, ether, and hexane to give the title compound as a beige solid. ¹H NMR (300 MHz, DMSO-d₆) δ 6.53 (s, 2H), 7.30 (d, J=8.4 Hz, 1H), 7.35 (s, 1H), 7.56 (m, 2H), 7.72 (s, 1H), 8.44 (s, 1H), 8.66 (s, 1H), 10.68 (s, 1H); LCMS: m−=338/340.

### Example 277

### (2E)-2-[(4-bromo-2-ethylphenyl)hydrazono]-2-cyanoacetamide

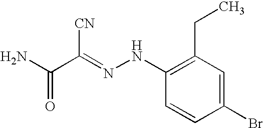

To 2-ethyl-4-bromoaniline (20.0 g, 100 mmol) was added conc. HCl (25 mL, 300 mmol) and water (100 mL). The stirred mixture was cooled in an ice-acetone bath and a solution on sodium nitrite (6.90 g, 100 mmol) in water (20 mL) was added over 20 min. To this solution was added at 0° C. a solution of sodium acetate trihydrate (40.8 g, 300 mmol) in water (100 mL). Separately cyanoacetamide (10.1 g, 120 mmol) was dissolved in water (180 mL) and ethanol (120 mL) then cooled in an ice-acetone bath. A solution of sodium acetate trihydrate (13.6 g, 100 mmol) in water (30 mL) was added. To this stirred mixture was added the diazotization mixture, dropwise over 30 min. After 1.5 h the the title compound (a yellow solid) was filtered and dried. ¹H NMR (300 MHz, DMSO-d₆) δ 1.18 (t, J=7.5 Hz, 3H), 2.76 (q, J=7.5 Hz, 2H), 7.43 (m, 3H), 7.70 (d, J=8.4 Hz, 1H), 7.81 (s, 1H), 10.47 (s, 1H).

### Example 278

### ethyl 4-amino-3-(aminocarbonyl)-1-(4-bromo-2-ethylphenyl)-1H-pyrazole-5-carboxylate

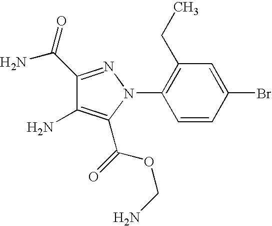

Ethyl bromoacetate (14.50 mL, 22.0 g, 131 mmol) was added to a stirred mixture of (2 E)-2-[(4-bromo-2-ethylphenyl)hydrazono]-2-cyanoacetamide (Example 277, 24.25 g, 82.2 mmol), anhydrous potassium carbonate (22.7 g, 164 mmol) and DMF (150 mL). The reaction was heated to 110° C. for 5 h then cooled to 90° C. when triethylamine (22.9 mL, 16.6 g, 164 mmol) was added. After cooling to 25° C. the mixture was poured into ice-water (1 L) and filtered. The precipitate was washed with methanol (4×50 mL) and dried to afford the title compound as a dirty yellow solid sufficiently pure for the next step. An analytical sample was obtained as a white solid by chromatography over silica gel (2% methanol-chloroform). ¹H NMR (300 MHz, DMSO-d₆) δ 0.99 (m, 6H), 2.26 (m, 2H), 4.05 (q, J=7.2 Hz, 2H), 5.67 (s, 2H), 7.24 (m, 1H), 7.30 (s, 1H), 7.49 (m, 1H), 7.59 (s, 2H); LCMS: m+=382/384.

### Example 279

### 4-amino-3-(aminocarbonyl)-1-(4-bromo-2-ethylphenyl)-1H-pyrazole-5-carboxylic acid, potassium salt

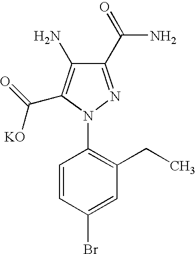

Ethyl 4-amino-3-(aminocarbonyl)-1-(4-bromo-2-ethylphenyl)-1H-pyrazole-5-carboxylate (Example 278, 11.98 g, 31.42 mmol) was stirred at 45° C. in 10% ethanolic (absolute) potassium hydroxide (100 mL) for 4.5 h then cooled in ice. The precipitate was filtered and washed with abs ethanol (2×50 mL), ether (50 mL) and hexane (50 mL) to afford the title compound as a yellow solid. ¹H NMR (300 MHz, DMSO-d₆) δ 0.94 (t, J=7.4 Hz, 3H), 2.26 (q, J=7.4 Hz, 2H), 5.26 (s, 2H), 6.94 (s, 1H), 7.01 (d, J=8.4 Hz, 1H), 7.16 (s, 1H), 7.35 (d, J=8.4 Hz, 1H), 7.42 (s, 1H); LCMS: m+=354/356 and m−=351/353.

### Example 280

### 4-amino-1-(4-bromo-2-ethylphenyl)-1H-pyrazole-3-carboxamide

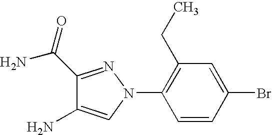

4-amino-3-(aminocarbonyl)-1-(4-bromo-2-ethylphenyl)-1H-pyrazole-5-carboxylic acid, potassium salt (Example 279, 7.608 g, 19.44 mmol) was stirred and heated to 75° C. with 85% phosphoric acid (100 mL) for 7 h then cooled in ice. Water (1600 mL) was added and the precipitate filtered and dried. The title compound was obtained as a pale yellow solid. ¹H NMR (300 MHz, DMSO-d₆) δ 0.99 (t, J=7.5 Hz, 3H), 2.55 (q, J=7.5 Hz, 2H), 7.16 (s, 1H), 7.30 (m, 3H), 7.52 (m, 1H), 7.60 (s, 1H); MS: m+=310/312.

### Example 281

### 4-[(aminocarbonyl)amino]-1-(4-bromo-2-ethylphenyl)-1H-pyrazole-3-carboxamide

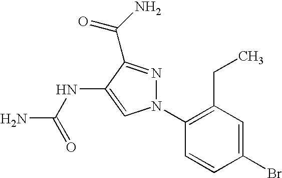

4-amino-1-(4-bromo-2-ethylphenyl)-1H-pyrazole-3-carboxamide (Example 280, 528 mg, 1.71 mmol) was stirred in a mixture of 2-propanol (7.5 mL), 75% acetic acid (7.5 mL) and water (7.5 mL). Potassium cyanate (152 mg, 1.88 mmol) was added over 1 min and the reaction stirred for 1 h then cooled in ice and water (80 mL) added. The precipitate was filtered and chromatographed over silica gel (5% methanol-chloroform) to afford the title compound as a white solid. ¹H NMR (300 MHz, DMSO-d₆) δ 0.98 (t, J=7.5 Hz, 3H), 2.54 (q, J=7.5 Hz, 2H), 6.48 (s, 2H), 7.33 (m, 1H), 7.43 (s, 1H), 7.50 (m, 1H), 7.63 (s, 2H), 8.11 (s, 1H), 8.689 (s, 1H); LCMS: m−=350/352.

### Example 282

### 4-[(aminocarbonyl)amino]-1-(3-ethyl-3′-hydroxy-1,1′-biphenyl-4-yl)-1H-pyrazole-3-carboxamide

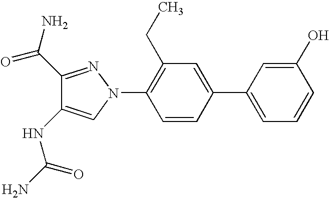

N₂ was bubbled through a mixture of 4-[(aminocarbonyl)amino]-1-(4-bromo-2-ethylphenyl)-1H-pyrazole-3-carboxamide (Example 281, 401 mg, 1.14 mmol), 3-hydroxyphenylboronic acid (314 mg, 2.28 mmol)) and Pd(PPh₃)₄ (197 mg, 0.171 mmol) in dry DMF(8 mL). A solution of Cs₂CO₃ (1.11 g, 3.42 mmol) in water (2.3 mL) was added and the mixture heated at 80° C. for 6 h. After cooling the mixture was poured into ice-water (100 mL) and filtered. The solid was chromatographed over silica gel (0-7% methanol-chloroform) to afford the title compound as a cream solid. ¹H NMR (300 MHz, DMSO-d₆) δ 1.04 (t, J=7.4 Hz, 3H), 2.61 (q, J=7.4 Hz, 2H), 6.48 (s, 2H), 6.78 (m, 1H), 7.06 (s, 1H), 7.12 (m, 1H), 7.26 (m, 1H), 7.42 (m, 2H), 7.53 (m, 1H), 7.61 (s, 2H), 8.14 (s, 1H), 8.69 (s, 1H), 9.56 (s, 1H);LCMS: m+=366, m−=364.

### Example 283

### 1-(3-carbamoyl-1-(4-(cyclopentylsulfinyl)phenyl)-1H-pyrazol-4-yl)urea

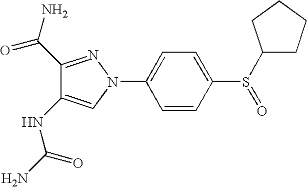

1-(3-carbamoyl-1-(4-(cyclopentylthio)phenyl)-1H-pyrazol-4-yl)urea (Example 207, 0.060 g, 0.16 mmol), glacial acetic acid (1 mL), and 30% hydrogen peroxide (0.018 mL, 0.18 mmol), were combined in a vial and the mixture stirred for 20 h at rt The solvent was then stripped off and the residue sonicated for 30 min. in 5.0 mL water. Then after settling, part of the supernatant was removed, the remaining water was stripped off, and the product was dried under vacuum. The title compound was an off white solid. ¹H NMR (DMSO-d₆): δ 1.38-1.64 (m, 5H), 1.70-1.90 (m, 3H), 6.51 (br s, 2H), 7.54 (br s, 1H), 7.74 (dm, 2H, J=8.7 Hz), 7.83 (br s, 1H), 8.07 (dm, 2H, J=8.9 Hz), 8.63 (s, 1H), 8.67 (s, 1H). [cyclopentyl H₁ appears to be overlapped by water, 3.29 δ, in DMSO based on another spectrum recorded in 60:40 acetonitrile:TFA]. Mass of Molecular Ion: 362 (M+1).

### Example 284

### 1-(3-carbamoyl-1-(4-(pyridin-2-ylsulfinyl)phenyl)-1H-pyrazol-4-yl)urea

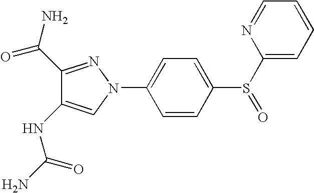

Prepared according to the procedure of Example 283, except the reaction was stirred for 45 h at rt. The title compound was an off white solid. ¹H NMR (DMSO-d₆): δ 6.50 (br s, 2H), 7.47-7.52 (m, 1H), 7.54 (br s, 1H), 7.76-7.85 (m, 3H), 7.95-8.11 (m, 4H), 8.57-8.62 (m, 2H), 8.65 (s, 1H). Mass of Molecular Ion: 371 (M+1).

### Example 285

### 1-(1-(4-(2-hydroxyphenylsulfinyl)phenyl)-3-carbamoyl-1H-pyrazol-4-yl)urea

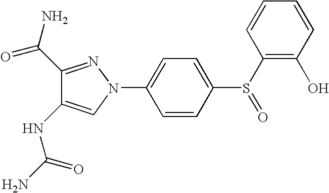

1-(1-(4-(2-hydroxyphenylthio)phenyl)-3-carbamoyl-1H-pyrazol-4-yl)urea (Example 208, 0.100 g, 0.15 mmol), glacial acetic acid (1.0 mL), and 30% hydrogen peroxide (0.017 mL, 0.17 mmol), were combined in a vial and the mixture stirred for 21 h at rt. Then glacial acetic acid (1.0 mL), and 30% hydrogen peroxide (0.017 mL, 0.17 mmol) were added and stirred for another 22 h at rt. The vial was then placed in a 100° C. oil bath for 10 min. The solvent was then stripped off, and the product purified by reverse phase preparative HPLC. The purified product was dried under vacuum. The title compound was a white solid. ¹H NMR (DMSO-d₆): δ 6.50 (br s, 2H), 6.84 (d, 1H, J=8.2 Hz), 6.99-7.04 (m, 1H), 7.27-7.33 (m, 1H), 7.53 (br s, 1H), 7.63 (dd, 1H, J=7.9, 1.7 Hz), 7.73-7.80 (m, 3H), 8.00 (dm, 2H, J=8.7 Hz), 8.57 (s, 1H), 8.65 (s, 1H), 10.59 (br s, 1H). Mass of Molecular Ion: 386 (M+1).

### Example 286

### 1-(3-carbamoyl-1-(4-(pyridin-2-ylsulfonyl)phenyl)-1H-pyrazol-4-yl)urea

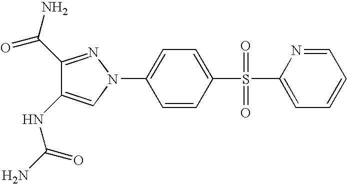

1-(3-carbamoyl-1-(4-(pyridin-2-ylthio)phenyl)-1H-pyrazol-4-yl)urea (Example 205, 0.014 g, 0.031 mmol), glacial acetic acid (0.30 mL), and 30% hydrogen peroxide (0.0090 mL, 0.088 mmol) were combined in a vial and then the vial was placed in an oil bath at 100C. After 2.0 h, 30% hydrogen peroxide (0.0040 mL, 0.039 mmol) was added, and the vial returned to the 100° C. oil bath for another 1.0 h. The solvent was then stripped off and the residue sonicated for 30 min. in 5.0 mL water. Then after settling, part of the supernatant was removed, the remaining water was stripped off, and the product was dried under vacuum. The title compound was a yellow solid. ¹H NMR (DMSO-d₆): δ 6.54 (br s, 2H), 7.59 (br s, 1H), 7.64-7.72 (m, 1H), 7.88 (br s, 1H), 7.98-8.26 (m, 6H), 8.63-8.73 (m, 3H). Mass of Molecular Ion: 387 (M+1).

### Example 287

### 1-(3-carbamoyl-1-(4-(cyclopentylsulfonyl)phenyl)-1H-pyrazol-4-yl)urea

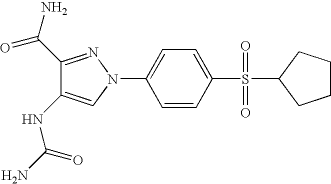

Prepared according to the procedure of Example 286, except the reaction was complete after 1.0 h and no additional peroxide was added. The title compound was a light yellow solid. ¹H NMR (DMSO-d₆): δ 1.47-1.66 (m, 4H), 1.73-1.90 (m, 4H), 3.76-3.85 (m, 1H), 6.54 (br s, 2H), 7.59 (br s, 1H), 7.90 (br s, 1H), 7.96 (dm, 2H, J=8.9 Hz), 8.16 (dm, 2H, J=8.9 Hz), 8.69 (s, 2H). Mass of Molecular Ion: 378 (M+1).

### Example 288

### 1-(1-(4-(2-hydroxyphenylsulfonyl)phenyl)-3-carbamoyl-1H-pyrazol-4-yl)urea

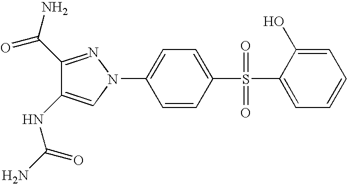

1-(1-(4-(2-hydroxyphenylthio)phenyl)-3-carbamoyl-1H-pyrazol-4-yl)urea (Example 208, 0.093 g, 0.14 mmol), glacial acetic acid (2 mL), and 30% hydrogen peroxide (0.043 mL, 0.42 mmol) were combined in a vial and then the vial was placed in an oil bath at 100° C. After 2.0 h, 30% hydrogen peroxide (0.014 mL, 0.14 mmol) was added, and the vial returned to the 100° C. oil bath for another 1.0 h. The solvent was then stripped off, the residue purified by reverse phase preparative HPLC, and the purified product was dried under vacuum. The title compound was a white solid. ¹H NMR (DMSO-d₆): δ 6.53 (br s, 2H), 6.89 (d, 1H, J=8.2 Hz), 6.98-7.03 (m, 1H), 7.45-7.51 (m, 1H), 7.58 (brs, 1H), 7.85 (brs, 1H), 7.91 (dd, 1H, J=7.9, 1.6 Hz), 7.97 (dm, 2H, J=9.0 Hz), 8.08 (dm, 2H, J=8.9 Hz), 8.64 (s, 1H), 8.67 (s, 1H), 10.82 (s, 1H). Mass of Molecular Ion: 402 (M+1).

### Example 289

### 1-(1-(4-(3-chlorophenylthio)phenyl)-3-carbamoyl-1H-pyrazol-4-yl)urea

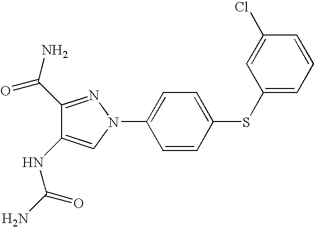

1-(3-carbamoyl-1-(4-iodophenyl)-1H-pyrazol-4-yl)urea (Example 215, 0.600 g, 1.62 mmol), copper (I) iodide (0.035 g, 0.18 mmol), (trans,6E,7E)—N1,N2-bis((pyridin-2-yl)methylene)cyclohexane-1,2-diamine (0.097 g, 0.33 mmol), cesium carbonate (0.536 g, 1.65 mmol), 4A molecular sieves (0.501 g), and anhydrous DMF (3.0 mL) were combined. The reaction flask was partially evacuated and backfilled with nitrogen three times, then 3-chlorobenzenethiol (0.28 mL, 2.4 mmol) was added and the flask placed in an oil bath at 110° C. Upon heating, the excess pressure was vented off. After 4.0 h at 110° C. the reaction was complete. The flask was then allowed to reach rt, 40 mL DMF was then added, the contents were filtered, and the solid was washed with 10 mL DMF. The DMF filtrate was then stripped of solvent, the residue sonicated for 30 min. in 75 mL water, filtered, and the solid washed with 25 mL water. The solid was then purified by reverse phase preparative HPLC, and the purified product was dried under vacuum. The title compound was a tan solid. ¹H NMR (DMSO-d₆): δ 6.50 (br s, 2H), 7.21 (dt, 1H, J=7.5, 1.5 Hz), 7.26 (t, 1H, J=1.7 Hz), 7.30-7.39 (m, 2H), 7.51-7.57 (m, 3H), 7.79 (br s, 1H), 7.94 (dm, 2H, J=8.7 Hz), 8.58 (s, 1H), 8.66 (s, 1H). Mass of Molecular Ion: 388 (M+1).

### Example 290

### 1-(1-(4-(4-chlorophenylthio)phenyl)-3-carbamoyl-1H-pyrazol-4-yl)urea

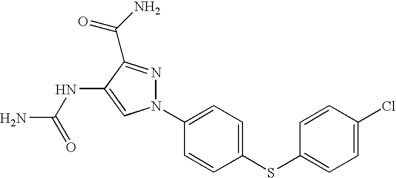

Prepared according to the procedure of Example 289, except the 4-chlorobenzenethiol was added to the flask before partial evacuation and backfilling with nitrogen. The title compound was a tan solid. ¹H NMR (DMSO-d₆): δ 6.50 (br s, 2H), 7.30 (dm, 2H, J=8.6 Hz), 7.42 (dm, 2H, J=8.6 Hz), 7.48 (dm, 2H, J=8.7 Hz), 7.51 (br s, 1H), 7.77 (br s, 1H), 7.91 (dm, 2H, J=8.7 Hz), 8.56 (s, 1H), 8.66 (s, 1H). Mass of Molecular Ion: 388 (M+1).

### Example 291

### 1-(3-carbamoyl-1-(4-(isopentylthio)phenyl)-1H-pyrazol-4-yl)urea

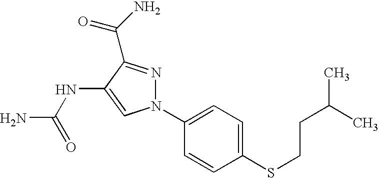

Prepared according to the procedure of Example 289, except that copper (I) iodide (0.061 g, 0.32 mmol), (trans,6E,7E)-N1,N2-bis((pyridin-2-yl)methylene)cyclohexane-1,2-diamine (0.190 g, 0.65 mmol), and isoamyl mercaptan (0.30 mL, 2.4 mmol) were used, and that the reaction ran for 6 h. The title compound was a tan solid. ¹H NMR (DMSO-d₆): δ 0.87 (d, 6H, J=6.7 Hz), 1.41-1.49 (m, 2H), 1.68 (sep, 1H, J=6.7 Hz), 2.95-3.01 (m, 2H), 6.48 (br s, 2H), 7.41 (dm, 2H, J=8.7 Hz), 7.48 (br s, 1H), 7.74 (br s, 1H), 7.80 (dm, 2H, J=8.7 Hz), 8.52 (s, 1H), 8.65 (s, 1H). Mass of Molecular Ion: 348 (M+1).

### Example 292

### 1-(1-(4-(m-tolylthio)phenyl)-3-carbamoyl-1H-pyrazol-4-yl)urea

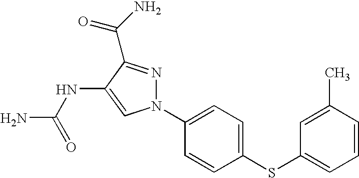

Prepared according to the procedure of Example 289. The title compound was a tan solid. ¹H NMR (DMSO-d₆): δ 2.26 (s, 3H), 6.50 (br s, 2H), 7.10-7.14 (m, 2H), 7.17 (s, 1H), 7.23-7.29 (m, 1H), 7.41 (dm, 2H, J=8.9 Hz), 7.50 (brs, 1H), 7.75 (br s, 1H), 7.87 (dm, 2H, J=8.9 Hz), 8.54 (s, 1H), 8.65 (s, 1H). Mass of Molecular Ion: 368 (M+1).

### Example 293

### 1-(1-(4-(3-bromophenylthio)phenyl)-3-carbamoyl-1H-pyrazol-4-yl)urea

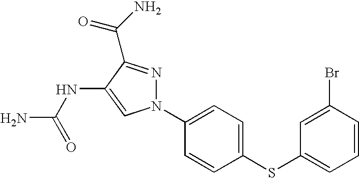

Prepared according to the procedure of Example 289. The title compound was a tan solid. ¹H NMR (DMSO-d₆): δ 6.50 (br s, 2H), 7.25 (dm, 1H, J=8.1 Hz), 7.30 (t, 1H, J=7.8 Hz), 7.40 (t, 1H, J=1.7 Hz), 7.45 (dm, 1H, J=7.7 Hz), 7.51-7.57 (m, 3H), 7.79 (br s, 1H), 7.94 (dm, 2H, J=8.7 Hz), 8.58 (s, 1H), 8.66 (s, 1H). Mass of Molecular Ion: 432 (M+1).

### Example 294

### 1-(3-carbamoyl-1-(4-(pyridin-4-ylsulfinyl)phenyl)-1H-pyrazol-4-yl)urea

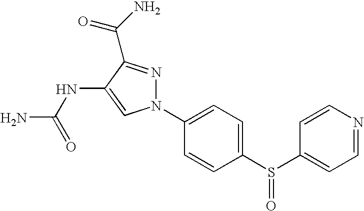

1-(3-carbamoyl-1-(4-(pyridin-4-ylthio)phenyl)-1H-pyrazol-4-yl)urea (Example 206, 0.050 g, 0.13 mmol), glacial acetic acid (1.0 mL), and 30% hydrogen peroxide (1.6 mL, 16 mmol) were combined in a vial and stirred at rt for 9.0 h. The reaction solution was then purified by reverse phase preparative HPLC, and the purified product was dried under vacuum. The title compound was a yellow solid. ¹H NMR (DMSO-d₆): δ 6.51 (br s, 2H), 7.55 (br s, 1H), 7.73 (dd, 2H, J=4.5, 1.5 Hz), 7.81 (br s, 1H), 7.89 (dm, 2H, J=8.9 Hz), 8.07 (dm, 2H, J=8.9 Hz), 8.60 (s, 1H), 8.65 (s, 1H), 8.71 (dd, 2H, J=4.6, 1.5 Hz). Mass of Molecular Ion: 371 (M+1).

### Example 295

### 1-(3-carbamoyl-1-(4-(pyridin-4-ylsulfonyl)phenyl)-1H-pyrazol-4-yl)urea

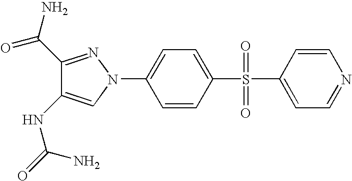

1-(3-carbamoyl-1-(4-(pyridin-4-ylthio)phenyl)-1H-pyrazol-4-yl)urea (Example 206, 0.050 g, 0.13 mmol), glacial acetic acid (1.0 mL), and 30% hydrogen peroxide (0.40 mL, 3.9 mmol) were combined in a vial and stirred at rt for 11.0 h, then 2 h at 55° C. The reaction solution was then purified by reverse phase preparative HPLC, and the purified product was dried under vacuum. The title compound was a yellow solid. ¹H NMR (DMSO-d₆): δ 6.53 (br s, 2H), 7.60 (br s, 1H), 7.90 (br s, 1H), 7.92 (dd, 2H, J=4.4, 1.6 Hz), 8.10 (dm, 2H, J=9.0 Hz), 8.17 (dm, 2H, J=9.0 Hz), 8.65-8.69 (m, 2H), 8.87 (dd, 2H, J=4.5, 1.6 Hz). Mass of Molecular Ion: 387 (M+1).

### Example 296

### 1-(2′-carbamoylmethoxy-4′-fluoro-2-methyl-biphenyl-4-yl)-4-ureido-1H-pyrazole-3-carboxylic acid amide

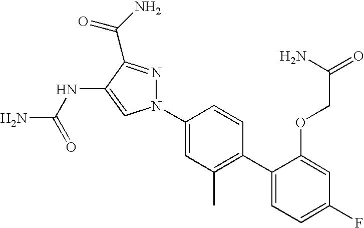

A slurry of 1-(4′-fluoro-2′-hydroxy-2-methyl-biphenyl-4-yl)-4-ureido-1H-pyrazole-3-carboxylic acid amide (Example 241, 75 mg, 0.20 mmol), 2-bromoacetamide (84 mg, 0.61 mmol) and potassium carbonate (56 mg, 0.41 mmol) in dry DMF (5 mL) was stirred at room temperature under nitrogen for 24 h. The reaction was concentrated in vacuo and the residue was re-suspended in water. The resulting precipitate collected via filtration and triturated with diethyl ether to give the title compound as an off-white solid. MS (ESI+) for C₂₀H₁₉FN₆O₄ m/z 427.1 (M+H)⁺. ¹H NMR (300 MHz, DMSO-d₆) δ 8.68 (s, 1H), 8.58 (s, 1H), 7.70-7.80 (m, 3H), 7.51 (s, 1H), 7.16-7.28 (m, 3H), 6.85-6.94 (m, 3H), 6.52 (brs, 2H), 5.44 (s, 2H), 2.16 (s, 3H).

### Example 297

### 1-[4-(4-fluoro-phenylsulfanyl)-3-methyl-phenyl]-4-ureido-1H-pyrazole-3-carboxylic acid amide

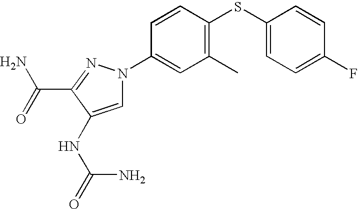

A slurry of 1-(4-iodo-3-methyl-phenyl)-4-ureido-1H-pyrazole-3-carboxylic acid amide (Example 8, 384 mg, 1.0 mmol), and copper(1) iodide (190 mg, 1.0 mmol) in DMF (8 mL) was degassed with N₂ for 10 min. Diisopropylethylamine (644 mg, 5.0 mmol) and ethylene glycol (309 mg, 5.0 mmol) were added, followed by 4-fluorothiophenol (256 mg, 2.0 mmol) and the reaction mixture heated to 90° C. for 24 h. The mixture was cooled to room temperature and concentrated in vacuo. The residue was triturated with water, filtered and the solids were slurried in THF. This mixture was filtered and the filtrate subjected to chromatography on silica gel (Biotage 40i, elution with 5% MeOH/chloroform). This provided a light brown solid that was triturated with dichloromethane to provide the title compound as an off-white solid. MS (ESI−) for C₁₈H₁₆FN₅O₂S m/z 384.0 (M−H)⁻. ¹H NMR (300 MHz, DMSO-d₆) δ 8.66 (s, 1H), 8.55 (s, 1H), 7.90 (d, J=2.1 Hz, 1H), 7.76 (s, 1H), 7.70 (dd, J=8.4, 2.1 Hz, 2H), 7.51 (s, 1H), 7.32-7.18 (m, 4H), 6.51 (brs, 2H), 2.54 (s, 3H).

### Example 298

### 1-[4-(4-fluoro-benzenesulfonyl)-3-methyl-phenyl]-4-ureido-1H-pyrazole-3-carboxylic acid amide

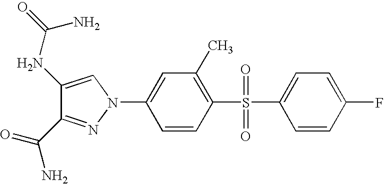

A slurry of 1-[4-(4-fluoro-phenylsulfanyl)-3-methyl-phenyl]-4-ureido-1H-pyrazole-3-carboxylic acid amide (Example 297, 108 mg, 0.28 mmol) and 3-chloroperoxybenzoic acid (150 mg, 0.84 mmol) in dichloromethane (5 mL) was stirred at room temperature for 24 h. The mixture was then diluted with THF (10 mL) and filtered. The precipitate was washed with DCM and air dried to give the title compound as a white solid. MS (ESI−) for C₁₈H₁₆FN₅O₄S m/z 416.0 (M−H)⁻. ¹H NMR (300 MHz, DMSO-d₆) δ 8.68 (s, 2H), 8.20 (d, J=8.4 Hz, 1H), 8.04-7.89 (m, 5H), 7.59 (s, 1H), 7.49-7.43 (m, 2H), 6.53 (brs, 2H), 2.56 (s, 3H).

### Example 299

### 1-(3-hydroxy-phenyl)-4-ureido-1H-pyrazole-3-carboxylic acid 4-methoxy-benzylamide

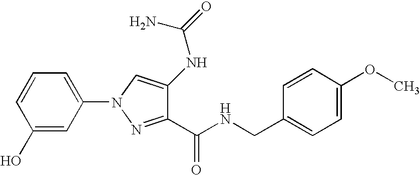

A slurry of 4-ureido-1H-pyrazole-3-carboxylic acid 4-methoxy-benzylamide (Example 321, Step 4, (100 mg, 0.35 mmol), copper(1) chloride (Strem 97%; 6.2 mg, 0.063 mmol), 3-hydroxyphenylboronic acid (58 mg, 0.42 mmol), pyridine (54 μL, 0.67 mmol) and anhydrous DMF (5 mL) was sealed under air in a capped 20 mL scintillation vial and heated to 80° C. for 24 h in a shaker block. The reaction was allowed to cool to rt and the solvent removed in vacuo. The residue was subjected to chromatography on silica gel (Biotage 40S, elution with 3% MeOH/CHCl₃) to give the title compound. MS (ESI−) for C₁₉H₁₉N₅O₄ m/z 380.0 (M−H)⁻. ¹H NMR (300 MHz, DMSO-d₆) δ 9.91 (s, 1H), 8.89 (m, 1H), 8.71 (s, 1H), 8.46 (s, 1H), 7.28-7.23 (m, 5H), 6.99-6.86 (m, 2H), 6.75-6.71 (m, 1H), 6.51 (brs, 2H), 4.38 (d, J=6.0 Hz, 2H), 3.95 (s, 3H).

### Example 300

### 1-(3-hydroxy-phenyl)-4-ureido-1H-pyrazole-3-carboxylic acid amide

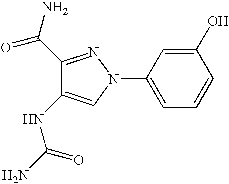

A solution of 1-(3-hydroxy-phenyl)-4-ureido-1H-pyrazole-3-carboxylic acid 4-methoxy-benzylamide (Example 299,169 mg, 0.44 mmol) in TFA (2 mL) was heated at 60° C. for 2 h. The resulting violet colored mixture was concentrated and subjected to chromatography on silica gel (Biotage 40S, elution with 4% MeOH/DCM). Concentration provided the title compound as an off-white solid. MS (ESI−) for C₁₁H₁₁N₅O₃ m/z 260.1 (M−H)⁻. ¹H NMR (300 MHz, DMSO-d₆) δ 9.89 (s, 1H), 8.65 (s, 1H), 8.44 (s, 1H), 7.71 (s, 1H), 7.49 (s, 1H), 7.29-7.21 (m, 3H), 6.74-6.71 (m, 1H), 6.50 (brs, 2H).

Examples 301-307 were prepared according to the same procedure as Example 216.

| | | ¹H NMR | MS |
|---|---|---|---|
| Example | Name and Structure | (500 MHz, CD₃OD) | (M + H) |
| 301 | 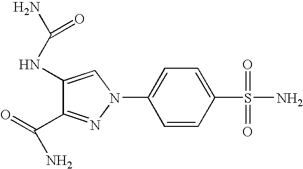 | m 6.51 (s, 2 H) 7.38 (s, 2 H) 7.58 (s, 1 H) 7.84 (s, 1 H) 7.90 (d, J=8.86 Hz, 2 H) 8.08 (d, J=8.86 Hz, 2 H) 8.65 (s, 1 H) 8.68 (s, 1 H) | 325.1 |
| | 4-[(aminocarbonyl)amino]-1-[4- | | |
| | (aminosulfonyl)phenyl]-1H-pyrazole-3- | | |
| | carboxamide | | |
| 302 | 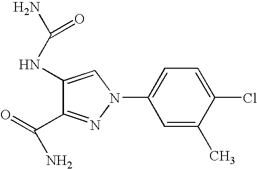 | 2.38 (s, 3 H) 6.50 (s, 2 H) 7.50 (m, 2 H) 7.73 (m, 1 H) 7.79 (m, 1 H) 7.92 (d, J=2.15 Hz, 1 H) 8.56 (s, 1 H) 8.66 (s, 1 H) | 294.1 |
| | 4-[(aminocarbonyl)amino]-1-(4-chloro-3- | | |
| | methylphenyl)-1H-pyrazole-3-carboxamide | | |
| 303 | 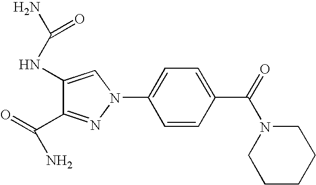 | 1.54 (m, 6 H) 3.30 (m, 4H) 6.48 (s, 2 H) 7.47 (m, 2 H) 7.52 (s, 1 H) 7.79 (s, 1 H) 7.92 (m,2 H) 8.59 (s, 1 H) 8.67 (s, 1 H) | 357.2 |
| | 4-[(aminocarbonyl)amino]-1-[4-(piperidin-1- | | |
| | ylcarbonyl)phenyl]-1H-pyrazole-3- | | |
| | carboxamide | | |
| 304 | 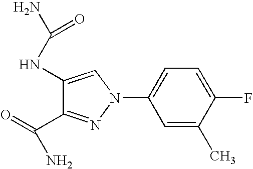 | 2.27 (s, 3 H) 6.46 (s, 2 H) 7.24 (t, J=9.13 Hz, 1 H) 7.47 (m, 1 H) 7.71 (m, 2 H) 7.82 (m, 1 H) 8.51 (s, 1 H) 8.65 (s, 1 H) | 278.2 |
| | 4-[(aminocarbonyl)amino]-1-(4-fluoro-3- | | |
| | methylphenyl)-1H-pyrazole-3-carboxamide | | |
| 305 | 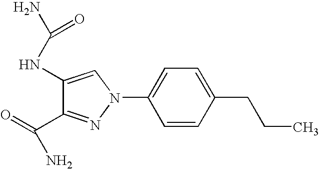 | 0.88 (t, J=7.25 Hz, 3 H) 1.56 (m, 2 H) 2.57 (m, 2 H) 6.48 (m, 2 H) 7.29 (d, J=8.59 Hz, 2 H) 7.45 (m,1 H) 7.70 (m,1 H) 7.74 (d, J=8.59 Hz, 2 H) 8.49 (s, 1 H) 8.64 (m, 1 H) | 288.2 |
| | 4-[(aminocarbonyl)amino]-1-(4-propylphenyl)- | | |
| | 1H-pyrazole-3-carboxamide | | |
| 306 | 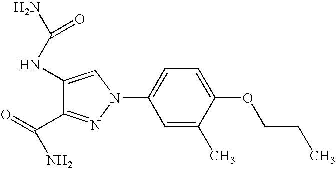 | 0.99 (t, J=7.38 Hz, 3 H) 1.78 (m, 2 H) 2.20 (s, 3 H) 3.95 (t, J=6.44 Hz, 2 H) 6.43 (s, 2 H) 6.99 (d, J=8.86 Hz, 1H) 7.44 (s, 1 H) 7.57 (m, 1 H) 7.67 (m, 2 H) 8.42 (s, 1 H) 8.64 (s, 1 H) | 318.2 |
| | 4-[(aminocarbonyl)amino]-1-(3-methyl-4- | | |
| | propoxyphenyl)-1H-pyrazole-3-carboxamide | | |
| 307 | 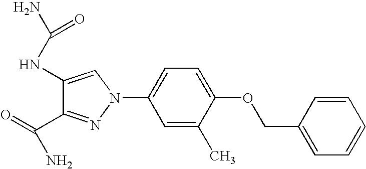 | | 366.2 |
| | 4-[(aminocarbonyl)amino]-1-[4-(benzyloxy)-3- | | |
| | methylphenyl]-1H-pyrazole-3-carboxamide | | |

### Example 308

### 1-(4′-hydroxy-2-methyl-biphenyl-4-yl)-4-ureido-1H-pyrazole-3-carboxylic acid amide

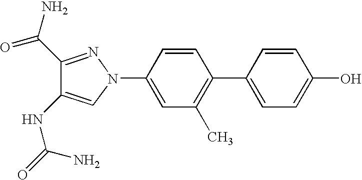

To a mixture of 1-(4-iodo-3-methyl-phenyl)-4-ureido-1H-pyrazole-3-carboxylic acid amide (Example 8, 384 mg, 0.997 mmol) and (PPh₃)₄Pd (173 mg, 0.150 mmol) in DMF (8 mL) was added (4-hydroxyphenyl)boronic acid (275 mg, 1.99 mmol). The mixture was sparged with nitrogen for 10 min and a solution of cesium carbonate (975 mg, 2.99 mmol) in water (2 mL) added. The mixture was sparged with nitrogen for an additional 10 min and heated in a sealed tube at 80° C. for 16 h. Volatiles were removed in vacuo and the residue purified by Biotage chromatography (40s silica gel column) using chloroform/CH₃OH (0-4%) as eluant to afford the title compound as a brown solid. ¹H NMR (300 MHz, DMSO-d₆) δ 1.21 (s, 3H), 6.51 (br s, 2H), 6.81 (d, J=8 Hz, 2H), 7.16 (d, J=8 Hz, 2H), 7.25 (d, J=8 Hz, 1H), 7.50 (s, 1H), 7.70 (d, J=8 Hz, 1H), 8.56 (s, 1H), 8.68 (s, 1H), 9.52 (s, 1H); MS (ESI−) for C₁₁H₁₇NO₃ m/z 350 (M−H)⁻.

### Example 309

### 1-(4′-hydroxy-2,2′-dimethyl-biphenyl-4-yl)-4-ureido-1H-pyrazole-3-carboxylic acid amide

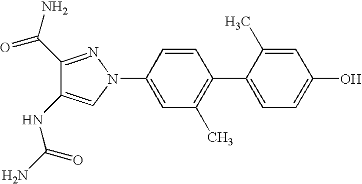

To a mixture of 1-(4-iodo-3-methyl-phenyl)-4-ureido-1H-pyrazole-3-carboxylic acid amide (Example 8, 384 mg, 0.997 mmol) and (PPh₃)₄Pd (173 mg, 0.150 mmol) in DMF (8 mL) was added (4-hydroxy-2-methylphenyl)boronic acid (151 mg, 1.00 mmol). The mixture was sparged with nitrogen for 10 min and a solution of cesium carbonate (975 mg, 2.99 mmol) in water (2 mL) added. The mixture was sparged with nitrogen for an additional 10 min and heated in a sealed tube at 80° C. for 16 h. Volatiles were removed in vacuo and the residue purified by Biotage chromatography (40s silica gel column) using chloroform/CH₃OH (0-4%) as eluant to afford the title compound as a tan solid. ¹H NMR (300 MHz, DMSO-d₆) δ 1.92 (s, 3H), 2.05 (s, 3H), 6.52 (br s, 2H), 6.61 (d, J=8 Hz, 1H), 6.65 (s, 1H), 6.85 (d, J=8 Hz, 1H), 7.18 (d, J=8 Hz, 1H), 7.46 (s, 1H), 7.65 (d, J=8 Hz, 1H), 7.78 (s, 2H), 8.56 (s, 1H), 8.64 (s, 1H), 9.34 (s, 1H); MS (ESI−) for C₁₉H₁₉N₅O₃ m/z 364 (M−H)⁻.

### Example 310

### 1-(3′-fluoro-4′-hydroxy-2-methyl-biphenyl-4-yl)-4-ureido-1H-pyrazole-3-carboxylic acid amide

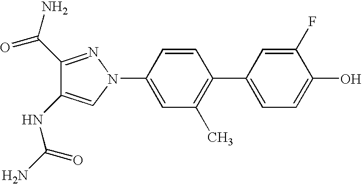

To a mixture of 1-(4-iodo-3-methyl-phenyl)-4-ureido-1H-pyrazole-3-carboxylic acid amide (Example 8, 384 mg, 0.997 mmol) and (PPh₃)₄Pd (173 mg, 0.150 mmol) in DMF (8 mL) was added (4-hydroxy-2-methylphenyl)boronic acid (155 mg, 1.00 mmol). The mixture was sparged with nitrogen for 10 min and a solution of cesium carbonate (975 mg, 2.99 mmol) in water (2 mL) added. The mixture was sparged with nitrogen for an additional 10 min and heated in a sealed tube at 80° C. for 16 h. Volatiles were removed in vacuo and the residue purified by Biotage chromatography (40s silica gel column) using chloroform/CH₃OH (0-6%) as eluant to afford the title compound as a brown solid. ¹H NMR (300 MHz, DMSO-d₆) δ 2.24 (s, 3H), 6.51 (brs, 2H), 6.99-7.01 (m, 2H), 7.16 (d, J=12 Hz, 1H), 7.28 (d, J=8 Hz, 1H), 7.50 (s, 1H), 7.72 (d, J=8 Hz, 1H), 7.77-7.79 (m, 2H), 8.57 (s, 1H), 8.67 (s, 1H), 9.95 (s, 1H); MS (ESI+) for C₁₈H₁₆FN₅O₃ m/z 368 (M+H)⁺.

### Example 311

### 1-[4-(3-fluoro-phenylsulfanyl)-3-methyl-phenyl]-4-ureido-1H-pyrazole-3-carboxylic acid amide

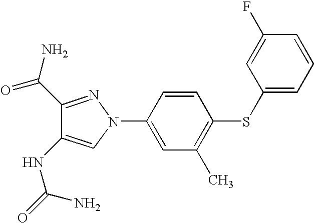

To a solution of 1-(4-iodo-3-methyl-phenyl)-4-ureido-1H-pyrazole-3-carboxylic acid amide (Example 8, 384 mg, 0.997 mmol) in DMF (8 mL) was added CuI (190 mg, 1.00 mmol). After sparging the mixture with nitrogen for 10 min, ethylene glycol (280 μL, 5.00 mmol), DIEA (870 μL, 5.00 mmol) and 3-fluorothiophenol (170 μL, 2.00 mmol) were added. The mixture was sparged with nitrogen for an additional 10 min and heated in a sealed tube at 105° C. for 16 h. Volatiles were removed in vacuo and the residue washed with cold 1% aqueous NaOH. The solid was purified by Biotage chromatography (40s silica gel column) using chloroform/CH₃OH (0-6%) as eluant to afford the title compound as a white solid. ¹H NMR (300 MHz, DMSO-d₆) δ 2.38 (s, 3H), 6.55 (br s, 2H), 6.89-6.91 (m, 2H), 7.01-7.03 (m, 1H), 7.32-7.34 (m, 1H), 7.51-7.53 (m, 3H), 7.81-7.83 (m, 2H), 7.99 (s, 1H), 8.62 (s, 1H), 8.68 (s, 1H); MS (ESI−) for C₁₈H₁₆FN O₂S m/z 384 (M−H)⁻.

### Example 312

### 1-[4-(3-fluoro-benzenesulfonyl)-3-methyl-phenyl]-4-ureido-1H-pyrazole-3-carboxylic acid amide

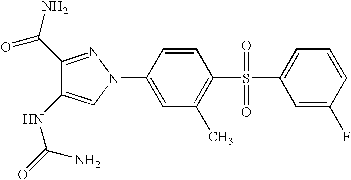

To a stirred mixture of 1-[4-(3-fluoro-phenylsulfanyl)-3-methyl-phenyl]-4-ureido-1H-pyrazole-3-carboxylic acid amide (Example 311, 70 mg, 0.18 mmol) in CH₂Cl₂ (5 mL) was added m-chloroperbenzoic acid (94 mg, 0.54 mmol). After 16 h, the reaction was diluted with THF (10 mL), filtered and washed with CH₂Cl₂. The combined filtrates were concentrated in vacuo and the residue purified by Biotage chromatography (40s silica gel column) using chloroform/CH₃OH (0-4%) as eluant to afford the title compound as a white solid. ¹H NMR (300 MHz, DMSO-d₆) δ 2.49 (s, 3H), 6.55 (br s, 2H), 7.48-7.77 (m, 7H), 7.89-7.93 (m, 2H), 8.04-8.10 (m, 2H), 8.23 (d, J=8 Hz, 1H), 8.69 (s, 2H); MS (ESI+) for C₁₈H₁₆FN₅O₄S m/z 418 (M+H)⁺.

### Example 313

### 1-(3-ethoxy-phenyl)-4-ureido-1H-pyrazole-3-carboxylic acid amide

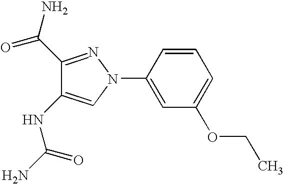

To a vial containing ammonium formate (260 mg, 4.12 mmol) and Pd/C (10% w/w, 100 mg) was added a solution of 1-(4-bromo-3-ethoxy-phenyl)-4-ureido-1H-pyrazole-3-carboxylic acid amide (Example 3, 100 mg, 0.272 mmol) in ethanol (3 mL) under an atmosphere of nitrogen. The vial was sealed and heated to 75° C. After 4 h, the mixture was cooled, filtered through a pad of celite and washed with ethanol. The combined filtrates were concentrated in vacuo and the residue partitioned between ethyl acetate and water. The organic layer was washed with water, brine, dried (Na₂SO₄), filtered and evaporated in vacuo to afford the title compound as a white solid. ¹H NMR (300 MHz, DMSO-d₆) δ 1.43 (t, J=7 Hz, 3H), 4.17 (q, J=7 Hz, 2H), 6.58 (br s, 2H), 6.92-6.96 (m, 1H), 7.41-7.45 (m, 2H), 7.52-7.59 (m, 2H), 7.90 (s, 1H), 8.62 (s, 1H), 8.72 (m, 1H); MS (ESI+) for C₁₃H₁₅N₅O₃ m/z 290 (M+H)⁺.

### Example 314

### 1-(2-ethyl-phenyl)-4-ureido-1H-pyrazole-3-carboxylic acid amide

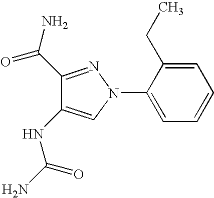

To a vial containing ammonium formate (269 mg, 4.26 mmol) and Pd/C (10% w/w, 100 mg) was added a solution of 1-(4-bromo-2-ethyl-phenyl)-4-ureido-1H-pyrazole-3-carboxylic acid amide (Example 281,100 mg, 0.284 mmol) in ethanol (3 mL) under an atmosphere of nitrogen. The vial was sealed and heated to 75° C. After 6 h, the mixture was cooled and filtered through a pad of celite and washed with ethanol. The combined filtrates were concentrated in vacuo and the residue partitioned between ethyl acetate and water. The organic layer was washed with water, brine, dried (Na₂SO₄), filtered and concentrated in vacuo. Purification of the residue by Biotage chromatography (40s silica gel column) using CH₂Cl₂/CH₃OH (0-5%) as eluant afforded the title compound as a white solid. ¹H NMR (300 MHz, DMSO-d₆) δ 0.99 (t, J=7 Hz, 3H), 2.50 (q, J=7 Hz, 2H), 6.48 (br s, 2H), 7.32-7.42 (m, 5H), 7.61 (s, 1H), 8.09 (s, 1H), 8.68 (s, 1H); MS (ESI−) for C₁₃H₁₅N₅O₂ m/z 272 (M−H)⁻.

### Example 315

### 1-m-tolyl-4-ureido-1H-pyrazole-3-carboxylic acid amide

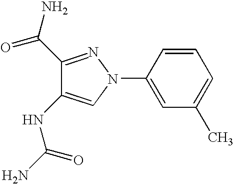

To a vial containing ammonium formate (340 mg, 4.26 mmol) and Pd/C (10% w/w, 140 mg) was added a solution of 1-(4-iodo-3-methyl-phenyl)-4-ureido-1H-pyrazole-3-carboxylic acid amide (Example 8, 140 mg, 0.360 mmol) in ethanol (3 mL) under an atmosphere of nitrogen. The vial was sealed and heated to 75° C. After 6 h, the mixture was cooled and filtered through a pad of celite and washed with ethanol. The combined filtrates were concentrated in vacuo and the residue partitioned between ethyl acetate and water. The organic layer was washed with water, brine, dried (Na₂SO₄), filtered and concentrated in vacuo. Purification of the residue by Biotage chromatography (40s silica gel column) using CH₂Cl₂/CH₃OH (0-4%) as eluant afforded the title compound as a tan solid. ¹H NMR (300 MHz, DMSO-d₆) δ 2.37 (s, 3H), 6.50 (br s, 2H), 7.14 (d, J=7 Hz, 1H), 7.36 (t, J=7 Hz, 1H), 7.49 (s, 1H), 7.64 (d, J=8 Hz, 1H), 7.70-7.77 (m, 1H), 8.54 (s, 1H), 8.66 (s, 1H); MS (ESI+) for C₁₂H₁₃N₅O₂ m/z 260 (M+H)⁺.

### Example 316

### 1-(3-fluoro-phenyl)-4-ureido-1H-pyrazole-3-carboxylic acid amide

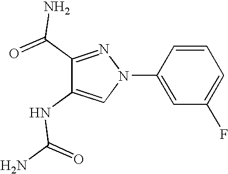

To a vial containing ammonium formate (553 mg, 8.77 mmol) and Pd/C (10% w/w, 100 mg) was added a solution of 1-(4-bromo-3-fluoro-phenyl)-4-ureido-1H-pyrazole-3-carboxylic acid amide (Example 225, 200 mg, 0.585 mmol) in ethanol (3 mL) under an atmosphere of nitrogen. The vial was sealed and shaken at ambient temperature. After 4 h, the mixture was cooled, filtered through a pad of celite and washed with ethanol. The combined filtrates were concentrated in vacuo and the residue partitioned between ethyl acetate and water. The organic layer was washed with water, brine, dried (Na₂SO₄), filtered and evaporated in vauco to afford the title compound as a white solid. ¹H NMR (300 MHz, DMSO-d₆) δ 6.53 (br s, 2H), 7.16 (t, J=8 Hz, 1H), 7.48-7.52 (m, 2H), 7.73 (d, J=8 Hz, 1H), 7.80-7.86 (m, 2H), 8.62 (s, 1H), 8.66 (s, 1H); MS (ESI−) for C₁₁H₁₀FN₅O₂ m/z 262 (M−H)⁻.

### Example 317

### 1-(3-trifluoromethyl-phenyl)-4-ureido-1H-pyrazole-3-carboxylic acid amide

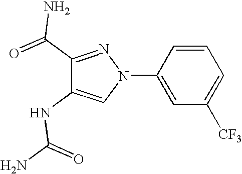

To a vial containing ammonium formate (180 mg, 2.90 mmol) and Pd/C (10% w/w, 125 mg) was added a solution of 1-(4-bromo-3-trifluoromethyl-phenyl)-4-ureido-1H-pyrazole-3-carboxylic acid amide (Example 1, 75 mg, 0.19 mmol) in ethanol (3 mL) under an atmosphere of nitrogen. The vial was sealed and shaken at ambient temperature. After 4 h, the mixture was cooled, filtered through a pad of celite and washed with ethanol. The combined filtrates were concentrated in vacuo and the residue partitioned between ethyl acetate and water. The organic layer was washed with water, brine, dried (Na₂SO₄), filtered and evaporated in vauco to afford the title compound as a white solid. ¹H NMR (300 MHz, DMSO-d₆) δ 6.53 (br s, 2H), 7.56 (s, 1H), 7.65-7.74 (m, 2H), 7.97 (s, 1H), 8.19 (d, J=7 Hz, 1H), 8.31 (s, 1H), 8.68 (s, 1H), 8.71 (s, 1H); MS (ESI−) for C₁₂H₁₀F₃N₅O₂ m/z 312 (M−H)—N

### Example 318

### 1-o-tolyl-4-ureido-1H-pyrazole-3-carboxylic acid amide

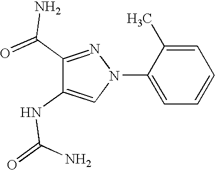

To a vial containing ammonium formate (250 mg, 3.90 mmol) and Pd/C (10% w/w, 100 mg) was added a solution of 1-(4-iodo-2-methyl-phenyl)-4-ureido-1H-pyrazole-3-carboxylic acid amide (Example 4,100 mg, 0.260 mmol) in ethanol (3 mL) under an atmosphere of nitrogen. The vial was sealed and heated to 75° C. After 6 h, the mixture was cooled and filtered through a pad of celite and washed with ethanol. The combined filtrates were concentrated in vacuo and the residue partitioned between ethyl acetate and water. The organic layer was washed with water, brine, dried (Na₂SO₄), filtered and concentrated in vacuo. Purification of the residue by Biotage chromatography (40s silica gel column) using CH₂Cl₂/CH₃OH (0-5%) as eluant afforded the title compound as a white solid. ¹H NMR (300 MHz, DMSO-d₆) δ 2.30 (s, 3H), 6.46 (br s, 2H), 7.25-7.43 (m, 6H), 7.64 (s, 1H), 8.10 (s, 1H), 8.67 (s, 1H); MS (ESI+) for C₁₂H₁₃N₅O₂Na m/z 282 (M+H)⁺.

### Example 319

### 1-(4-ethoxy-phenyl)-4-ureido-1H-pyrazole-3-carboxylic acid 4-methoxy-benzylamide

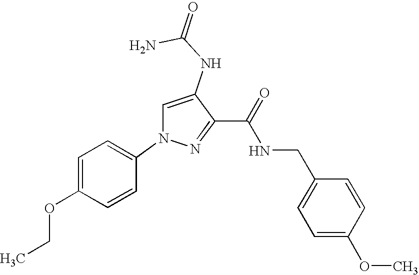

To a vial containing 4-ureido-1H-pyrazole-3-carboxylic acid 4-methoxy-benzylamide (Example 321, Step 4, 350 mg, 1.21 mmol), CuI (46 mg, 0.24 mmol), rac-trans-1,2-bis(2′-pyridylidenamino)-cyclohexane (prepared as described by Cristau et al., Org. Lett. 2004, 6, 913.) (141 mg, 0.484 mmol) and cesium carbonate (0.789 mg, 2.42 mmol) was added a solution of 4-iodophenetole (345 mg, 1.39 mmol) in DMF (3 mL). The mixture was sparged with nitrogen for 10 min, sealed and heated at 100C for 16 h. Volatiles were removed in vacuo and the residue purified by Biotage chromatography (40M silica gel column) using hexane/ethyl acetate (20-60%) containing 0.1% isopropanol as eluant to afford the title compound as a white solid. ¹H NMR (300 MHz, CDCl₃) δ 1.41 (t, J=7 Hz, 3H), 3.72 (s, 3H), 4.04 (q, J=7 Hz, 2H), 4.50-4.54 (m, 2H), 5.11 (br s, 2H), 6.84-6.92 (m, 4H), 7.18-7.30 (m, 3H), 7.53-7.58 (m, 2H), 8.42 (s, 1H), 8.89 (s, 1H); MS (ESI+) for C₂₁H₂₃N₅O₄ m/z 410 (M+H)⁺.

### Example 320

### 1-pyridin-2-yl-4-ureido-1H-pyrazole-3-carboxylic acid 4-methoxy-benzylamide

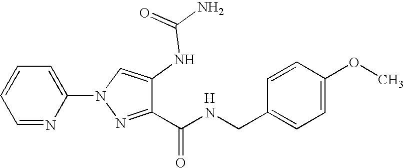

To a vial containing 4-ureido-1H-pyrazole-3-carboxylic acid 4-methoxy-benzylamide (Example 321, Step 4, 500 mg, 1.73 mmol), CuI (66 mg, 0.35 mmol), rac-trans-1,2-bis(2′-pyridylidenamino)-cyclohexane (202 mg, 0.69 mmol) and cesium carbonate (1.130 g, 3.46 mmol) was added a solution of 2-iodopyridine (407 mg, 1.99 mmol) in DMF (8 mL). The mixture was sparged with nitrogen for 10 min, sealed and heated at 100° C. for 16 h. Volatiles were removed in vacuo and the residue purified by Biotage chromatography (40M silica gel column) using CH₂Cl₂/CH₃OH (0-5%) as eluant to afford the title compound as a white solid. ¹H NMR (300 MHz, CDCl₃) δ 3.81 (s, 3H), 4.48-4.58 (m, 2H), 4.86 (br s, 2H), 6.93 (d, J=8 Hz, 2H), 7.20-7.36 (m, 4H), 7.75-7.85 (m, 2H), 8.42-8.45 (m, 1H), 8.82 (s, 1H), 9.08 (s, 1H); MS (ESI+) for C₁₈H₁₈N₆O₃ m/z 367 (M+H)⁺.

### Example 321

### 4-[(aminocarbonyl)amino]-N-(4-methoxybenzyl)-1-(2-methylpyridin-4-yl)-1H-pyrazole-3-carboxamide

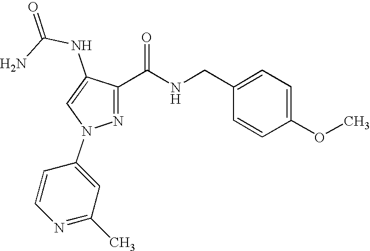

### Step 1: Preparation of ethyl 4-nitro-1H-pyrazole-3-carboxylate

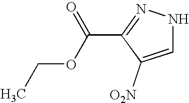

4-Nitro-1H-pyrazole-3-carboxylic acid (2 g, 12.73 mmol; Aldrich) was refluxed in absolute ethanol (25 mL) containing trace sulfuric acid (0.2 mL) for 5 h. The reaction was evaporated and partitioned between EtOAc and 5% NaHCO₃. The EtOAc layer was separated, dried over MgSO₄ and filtered. The EtOAc was removed to give the title compound as a white solid. ¹H NMR (300 MHz, DMSO-d₆) δ 1.28 (t, J=7 Hz, 3H), 4.34 (q, J=7 Hz, 2H), 8.91 (s, 1H), 14.39 (br s, 1H); MS (ESI+) for C₆H₇N₃O₄ m/z 186.1 (M+H)⁺.

### Step 2: Preparation of N-(4-methoxybenzyl)-4-nitro-1H-pyrazole-3-carboxamide

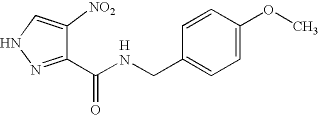

Ethyl 4-nitro-1H-pyrazole-3-carboxylate (Step 1, 0.100 g, 0.54 mmol) was stirred in excess 4-methoxybenzylamine at 120° C. for 0.5 h. The resulting product was triturated with EtOAc and filtered to give the title compound as the 4-methoxybenzylamine salt (1:1). ¹H NMR (300 MHz, DMSO-d₆) δ 3.72 (s, 3H), 3.73 (s, 3H), 3.81 (s, 2H), 4.36 (d, J=6 Hz, 2H), 5.9 (br s, 3H), 6.89 (m, 4H), 7.28 (m, 4H), 8.235 (s, 1H), 8.79 (m, 1H); MS (ESI+) for C₁₂H₁₂N₄O₄ m/z 277.1 (M+H)⁺.

### Step 3: Preparation of N-(4-methoxybenzyl)-4-amino-1H-pyrazole-3-carboxamide

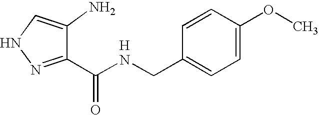

The salt from Step 2 (0.350 g, 1.27 mmol) was hydrogenated using 10% Pd/C (0.08 g) in EtOH/THF (8 mL, 1:1) at atmospheric pressure (H₂ balloon) for 72 h. The reaction was filtered through Celite and evaporated to give the product as a solid. ¹H NMR (300 MHz, DMSO-d₆) δ 3.70 (s, 3H), 4.13 (d, 1H), 4.31 (d, 1H), 4.60 (br s, 1H), 6.28 (m, 1H), 6.86 (m, 2H), 7.18 (m, 2H), 8.3 (s, 1H), 12.6 (s, 1H); MS (ESI+) for C₁₂H₁₄N₄O₂ m/z 247.1 (M+H)⁺.

### Step 4: Preparation of 4-[(aminocarbonyl)amino]-N-(4-methoxybenzyl)-1H-pyrazole-3-carboxamide

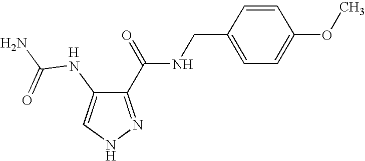

The product from Step 3 (0.188 g, 0.763 mmol) and sodium cyanate (0.099 g, 1.52 mmol) were suspended in water (1 mL). Tetrahydrofuran (1 mL) was added, followed by HOAc (0.5 mL) and the reaction was stirred at rt for 10 min. The reaction was evaporated and partitioned between EtOAc and water, the EtOAc layer was separated and evaporated to give the title compound as an oil. The product was purified on silica gel, eluting with 5% MeOH/CH₂Cl₂ to give the title compound. ¹H NMR (300 MHz, DMSO-d₆) δ 3.7 (s, 3H), 4.32 (d, 2H), 6.30 (br s, 2H), 6.84 (d, 2H), 7.22 (d, 2H), 7.92 (s, 1H), 8.62 (m, 2H), 12.89 (s, 1H); MS (ESI+) for C₁₃H₁₅NNO₃ m/z 290.0 (M+H)⁺.

### Step 5: Preparation of 4-[(aminocarbonyl)amino]-N-(4-methoxybenzyl)-1-(2-methylpyridin-4-yl)-1H-pyrazole-3-carboxamide

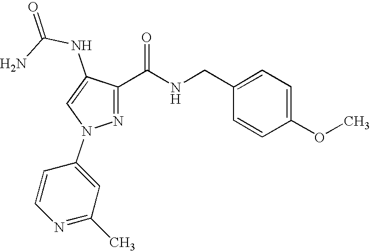

4-[(aminocarbonyl)amino]-N-(4-methoxybenzyl)-1H-pyrazole-3-carboxamide from Step 4 (0.150 g, 0.518 mmol), 2-picoline-4-boronic acid (0.106 g, 0.78 mmol), copper(1) chloride (0.010 g, 0.10 mmol) and pyridine (0.062 g, 0.78 mmol) were combined in DMF (3 mL) and heated overnight at 80° C. The reaction was cooled to room temperature, filtered through Celite and evaporated to give an oil. The product was purified on silica gel, eluting with 5% MeOH/CH₂Cl₂, to give the title compound as an oil. ¹H NMR (300 MHz, CDCl₃) δ 2.58 (s, 3H), 3.48 (s, 1H), 3.78 (s, 3H), 4.53 (d, 2H), 5.31 (s, 2H), 6.87 (d, 2H), 7.27 (m, 2H), 7.39 (d, 1H), 7.47 (s, 1H), 8.48 (d, 1H), 8.65 (s, 1H), 8.91 (s, 1H); MS (ESI+) for C₁₉H₂₀N₆O₃ m/z 381.1 (M+H)⁺.

### Example 322

### 4-[(aminocarbonyl)amino]-1-(2-methylpyridin-4-yl)-1H-pyrazole-3-carboxamide

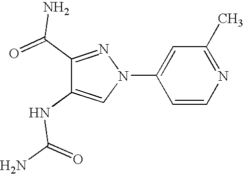

4-[(aminocarbonyl)amino]-N-(4-methoxybenzyl)-1-(2-methylpyridin-4-yl)-1H-pyrazole-3-carboxamide (Example 321, 0.022 g, 0.048 mmol) was stirred in neat trifluoroacetic acid at 60° C. for 4 h and evaporated to give the product as a foam. The material was partitioned between ethyl acetate and saturated sodium bicarbonate, the layers were separated and the ethyl acetate layer was evaporated to give the title compound. ¹H NMR (300 MHz, CDCl₃with 1 drop CD₃OD) δ 2.59 (s, 3H), 7.28 (m, 2H), 8.42 (d, 1H), 8.58 (s, 1H), 8.63 (s, 1H); MS (ESI+) for C₁₁H₁₂N₆O₂ m/z261.1 (M+H)⁺.

### Example 323

### 4-[(aminocarbonyl)amino]-1-dibenzo[b,d]thien-4-yl-N-(4-methoxybenzyl)-1H-pyrazole-3-carboxamide

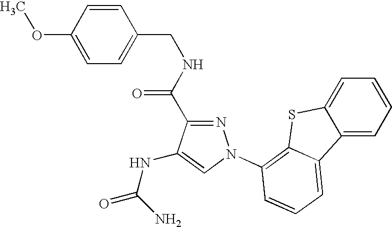

Prepared according to the same procedure as Example 321, using 4-dibenzothiopheneboronic acid (0.177 g) to give the title compound as a solid. ¹H NMR (300 MHz, CDCl₃) δ 3.81 (s, 3H), 4.64 (d, 2H), 4.9 (br s, 2H), 6.91 (d, 2H), δ 7.34 (m, 3H), 7.48 (m, 3H), 7.66 (d, 1H), 7.83 (m, 1H), 8.13 (m, 2H), 8.72 (s, 1H), 8.92 (s, 1H); MS (ESI+) for C₂₁H₂₁N₅O₃S m/z 472.1 (M+H)⁺.

### Example 324

### 4-[(aminocarbonyl)amino]-1-dibenzo[b,d]thien-4-yl-1H-pyrazole-3-carboxamide

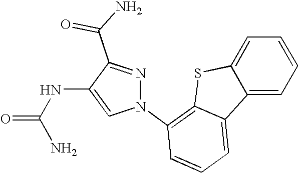

Prepared from 4-[(aminocarbonyl)amino]-1-dibenzo[b,d]thien-4-yl-N-(4-methoxybenzyl)-1H-pyrazole-3-carboxamide (Example 323) according to the same procedure as Example 322. The material was triturated from MeOH to give the title compound. ¹H NMR (300 MHz, DMSO-d₆) δ 6.57 (br s, 2H), 7.37 (s, 1H), 7.56 (m, 2H), 7.64 (m, 1H), 7.77 (s, 1H), 7.96 (d, 1H), 8.05 (d, 1H), 8.39 (m, 2H), 8.75 (d, 2H); MS (ESI+) for C₁₇H₁₃N₅O₂S m/z 352.1

### Example 325

### 4-[(aminocarbonyl)amino]-1-(3-fluoro-4-methylphenyl)-N-(4-methoxybenzyl)-1H-pyrazole-3-carboxamide

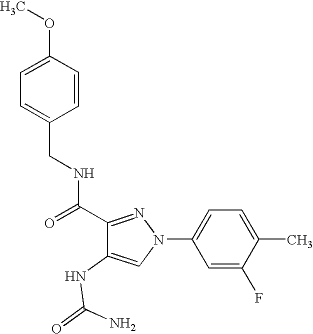

Prepared according to the same procedure as Example 321, using (3-fluoro-4-methylphenyl)boronic acid (0.12 g) to give the title compound as a solid. ¹H NMR (300 MHz, CDCl₃) δ 2.28 (s, 3H), 3.49 (d, 1H), 3.80 (s, 3H), 4.55 (d, 2H), 4.87 (s, 2H), 6.89 (d, 2H), 7.29 (m, 5H), 8.48 (s, 1H), 8.87 (s, 1H); MS (ESI+) for C₂₀H₂₀FN₅O₃ m/z 398.1

### Example 326

### 4-[(aminocarbonyl)amino]-1-(3-fluoro-4-methylphenyl)-1H-pyrazole-3-carboxamide

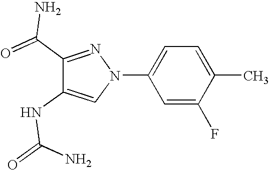

Prepared from 4-[(aminocarbonyl)amino]-1-(3-fluoro-4-methylphenyl)-N-(4-methoxybenzyl)-1H-pyrazole-3-carboxamide (Example 325) according to the same procedure as Example 322. The material was triturated from methylene chloride to give the title compound as a solid. ¹H NMR (300 MHz, DMSO-d₆) δ 2.24 (s, 3H), 6.6 (br s, 2H), 7.37 (m, 1H), 7.51 (s, 1H), 7.62 (d, 1H), 7.78 (m, 2H), 8.56 (s, 1H), 8.65 (s, 1H); MS (ESI+) for C₁₂H₁₂FN₅O₂ m/z 278.1.

### Example 327

### 4-[(aminocarbonyl)amino]-N-(4-methoxybenzyl)-1-[4-(trifluoromethoxy)phenyl]-1H-pyrazole-3-carboxamide

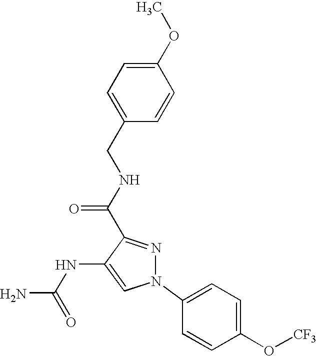

Prepared according to the same procedure as Example 321, using 4-(trifluoromethoxy)phenylboronic acid (0.16 g) to give the title compound. ¹H NMR (300 MHz, CDCl₃) δ 3.78 (s, 3H), 4.52 (d, 2H), 5.32 (s, 2H), 6.88 (d, 2H), 7.26 (m, 5H), 7.67 (d, 2H), 8.52 (s, 1H), 8.95 (s, 1H); MS (ESI+) for C₂₀H₁₈F₃N₅O₄ m/z 450.1.

### Example 328

### 4-[(aminocarbonyl)amino]-1-[4-(trifluoromethoxy)phenyl]-1H-pyrazole-3-carboxamide

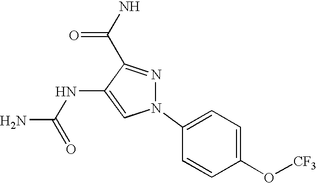

Prepared from 4-[(aminocarbonyl)amino]-N-(4-methoxybenzyl)-1-[4-(trifluoromethoxy)phenyl]-1H-pyrazole-3-carboxamide (Example 327) according to the same procedure as Example 322 to give the title compound as a solid. ¹H NMR (300 MHz, DMSO-d₆) δ 6.5 (br s, 2H), 7.51 (m, 3H), 7.8 (s, 1H), 8.01 (d, 2H), 8.59 (s, 1H), 8.67 (s, 1H); MS (ESI+) for C₁₂H₁₀F₃N₅O₃ m/z 330.1.

### Example 329

### 4-[(aminocarbonyl)amino]-1-(3,4-dimethoxyphenyl)-N-(4-methoxybenzyl)-1H-pyrazole-3-carboxamide

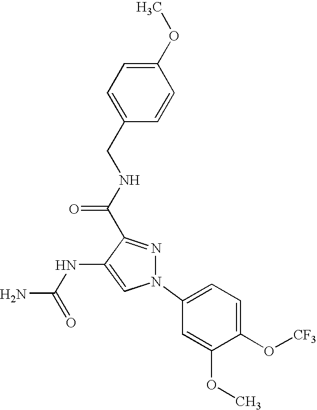

Prepared according to the same procedure as Example 321, using 3,4-dimethoxyphenylboronic acid (0.141 g) to give the title compound. ¹H NMR (300 MHz, CDCl₃) δ 3.80 (s, 3H), 3.91 (s, 3H), 3.93 (s, 3H), 4.56 (d, 2H), 4.81 (br s, 2H), 6.89 (m, 3H), 7.26 (m, 5H), 8.44 (s, 1H), 8.95 (br s, 1H); MS (ESI+) for C₂₁H₂₃N₅O₅ m/z 426.2.

### Example 330

### 4-[(aminocarbonyl)amino]-1-(3,4-dimethoxyphenyl)-1H-pyrazole-3-carboxamide

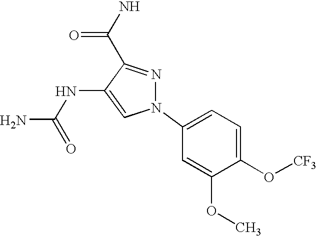

Prepared from 4-[(aminocarbonyl)amino]-1-(3,4-dimethoxyphenyl)-N-(4-methoxybenzyl)-1H-pyrazole-3-carboxamide (Example 329) according to the same procedure as Example 322. The material was triturated from ethyl acetate to 5 give the title compound. ¹H NMR (300 MHz, DMSO-d₆) δ 3.77 (s, 3H), 3.85 (s, 3H), 6.5 (br s, 2H), 7.02 (d, 1H), 7.35 (m, 1H), 7.48 (m, 2H), 7.78 (s, 1H), 8.48 (s, 1H), 8.64 (s, 1H); MS (ESI+) for C₁₃H₁₅N₅O₄ m/z 306.25.

### Example 331

### 4-[(aminocarbonyl)amino]-N-(4-methoxybenzyl)-1-(4-propoxyphenyl)-1H-pyrazole-3-carboxamide

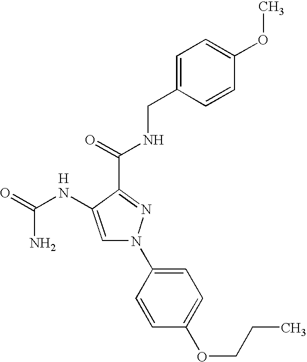

Prepared according to the same procedure as Example 321, using 4-propoxyphenylboronic acid (0.14 g) to give the title compound. ¹H NMR (300 MHz, CDCl₃) δ 1.05 (t, 3H), 1.81 (m, 2H), 3.80 (s, 3H), 3.94 (m, 2H), 4.55 (d, 2H), 4.8 (br s, 2H), 6.90 (m, 4H), 7.26 (m, 3H), 7.57 (d, 2H), 8.42 (s, 1H), 8.9 (br s, 1H); MS (ESI+) for C₂₂H₂₅N₅O₄ m/z 424.37.

### Example 332

### 4-[(aminocarbonyl)amino]-1-(4-propoxyphenyl)-1H-pyrazole-3-carboxamide

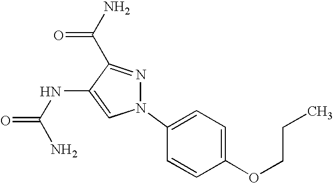

Prepared from 4-[(aminocarbonyl)amino]-N-(4-methoxybenzyl)-1-(4-propoxyphenyl)-1H-pyrazole-3-carboxamide (Example 331) according to the same procedure as Example 322. The material was recrystallized from MeOH to give the title compound. ¹H NMR (300 MHz, DMSO-d₆) δ 0.97 (t, 3H), 1.74 (m, 2H), 3.95 (m, 2H), 6.48 (br s, 2H), 7.02 (d, 2H), 7.45 (s, 1H), 7.73 (m, 3H), 8.44 (s, 1H), 8.65 (s, 1H); MS (ESI+) for C₁₄H₁₇N₅O₃ m/z 304.28.

### Example 333

### 4-[(aminocarbonyl)amino]-1-(3-chloro-4-propoxyphenyl)-N-(4-methoxybenzyl)-1H-pyrazole-3-carboxamide

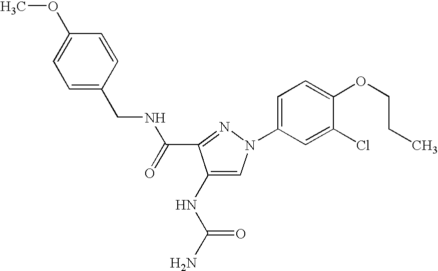

Prepared according to the same procedure as Example 321, using 3-chloro-4-propoxyphenylboronic acid (0.167 g) to give the title compound. ¹H NMR (300 MHz, CDCl₃) δ 1.08 (t, 3H), 1.87 (m, 2H), 3.80 (s, 3H), 4.01 (m, 2H), 4.55 (d, 2H), 4.81 (br s, 2H), 6.91 (m, 3H), 7.26 (m, 3H), 7.45 (m, 1H), 7.77 (m, 1H), 8.42 (s, 1H), 8.9 (br s, 1H); MS (ESI+) for C₂₂H₂₄ClN₅O₄ m/z 458.29.

### Example 334

### 4-[(aminocarbonyl)amino]-1-(3-chloro-4-propoxyphenyl)-1H-pyrazole-3-carboxamide

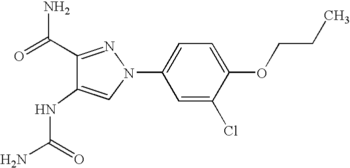

Prepared from 4-[(aminocarbonyl)amino]-1-(3-chloro-4-propoxyphenyl)-N-(4-methoxybenzyl)-1H-pyrazoie-3-carboxamide (Example 333) according to the same procedure as Example 322. The material was recrystallized from MeOH to give the title compound. ¹H NMR (300 MHz, DMSO-d₆) δ 0.997 (t, 3H), 1.75 (m, 2H), 4.05 (m, 2H), 6.49 (br s, 2H), 7.22 (d, 1H), 7.47 (s, 1H), 7.78 (m, 2H), 8.02 (d, 1H), 8.51 (s, 1H), 8.65 (s, 1H); MS (ESI+) for C₁₄H₁₆ClN₅O₃ m/z 338.28.

Examples 335 and 336: Prepared according to the same procedure as Examples 252-257.

| | | ¹H NMR | MS |
|---|---|---|---|
| Example | Name and Structure | (500 MHz, CD₃OD) | (M + H) |
| 335 | 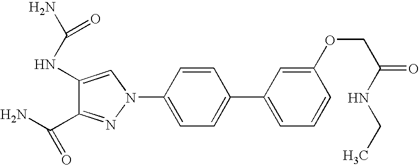 | 1.04 (t, 3H, J=7.2 Hz), 3.18 (quintet, 2H, J=7.1 Hz), 4.58 (s, 2H), 6.53 (br s, 2H), 6.98 (d 1H, J=9.1 Hz), 7.28-7.45 (m, 1 H), 7.53 (br s, 1H), 7.76-7.86 (m, 3H), 7.95 (d, 2H, J=8.7 Hz), 8.08-8.18 (m, 1H), 8.62 (s, 1H, 8.69 (s, 1H) | 423 |
| | 4-[(aminocarbonyl)amino]-1-{3′-[2-(ethylamino)-2- | | |
| | oxoethoxy]-1, 1′-biphenyl-4-yl}-1H-pyrazole-3- | | |
| | carboxamide | | |
| 336 | 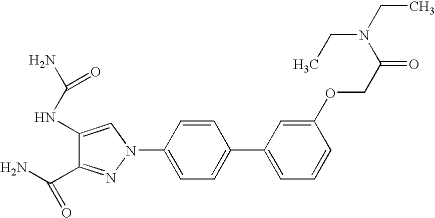 | 0.96-1.14 (m, 6H), 3.20-3.42 (m, 4H), 4.92 (s, 2H), 6.54(br s, 2H), 6.92(d, 1H, J=8.0 Hz), 7.20 (s, 1H), 7.38 (d, 1H, J=7.8 Hz), 7.47 (t, 1H, J=8.0 Hz), 7.54 (s, 1H), 7.75-7.85 (m, 3H), 7.96 (d, 2H, J=8.8 Hz), 8.62 (s, 1H), 8.69 (s, 1H) | 451 |
| | 4-[(aminocarbonyl)amino]-1-{3′-[2-(diethylamino)- | | |
| | 2-oxoethoxy]-1,1′-biphenyl-4-yl}-1H-pyrazole-3- | | |
| | carboxamide | | |

### Example 337

### 4-{3-(aminocarbonyl)-4-[(aminocarbonyl)amino]-1H-pyrazol-1-yl}phenylboronic acid

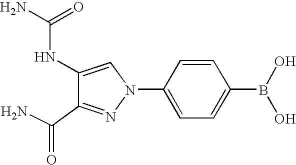

[(4′-{3-(aminocarbonyl)-4-[(aminocarbonyl)amino]-1H-pyrazol-1-yl}-5-fluoro-1,1′-biphenyl-2-yl)oxy]acetic acid (Example 259, 1.42 g) was partially dissolved in 30 mL of conc. HCl and stirred for 1 h; the liquid was removed under reduced pressure. The solid was triturated with CHCl₃, filtered, and dried under reduced pressure to give the desired product. ¹H NMR DMSO-d₆: δ 6.51 (br s, 2H), 7.50 (s, 1H), 7.78(s, 1H), 7.85 (d, 2H, J=8.6 Hz), 7.90 (d, 2H, J=8.6 Hz), 8.12 (brs 2H), 8.58 (s, 1H), 8.67 (s, 1H). Mass of Molecular Ion: 290

### Example 338

### 4-[(aminocarbonyl)amino]-1-[3′-(3-amino-3-oxopropyl)-1,1′-biphenyl-4-yl]-1H-pyrazole-3-carboxamide

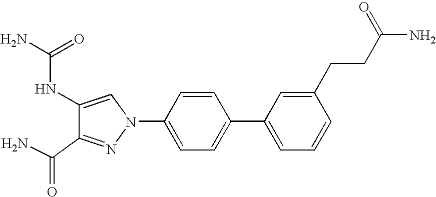

Prepared according to the procedure of Example 183, except 4-[(aminocarbonyl)amino]-1-(3′-{3-[(2,4-dimethoxybenzyl)amino]-3-oxopropyl}-1,1′-biphenyl-4-yl)-1H-pyrazole-3-carboxamide (Example 380, 0.2 mmol) was used as a starting material. ¹H NMR (CD₃OD)/DMSO-d₆(4:1): δ 2.40 (t, 2H, J=7.4 Hz), 2.85 (t, 2H, J=7.4 Hz), 6.53 (brs, 2H), 7.17 (d, 1H, J=7.6 Hz), 7.28 (t, 1H, J=7.6 Hz), 7.38-7.48 (m, 2H), 7.68 (d, 2H, J=8.7 Hz), 7.82 (d, 2H, J=8.7 Hz), 8.49 (s, 1H). Mass of Molecular Ion: 393.

Examples 339-362: Prepared according to the procedure of Examples 21-95 with the following exceptions: 4-[(aminocarbonyl)amino]-1-(3-bromophenyl)-1H-pyrazole-3-carboxamide (Example 20, 0.075 g, 0.231 mmol), Pd(OAc)₂(2.1 mg, 0.009 mmol), PPh3 (poly-supp) (11.5 mg, 0.018 mmol), 0.303 mmol of an arylboronic acid, DMF (2 mL) and 0.3 mL of 2M Cs2CO3 solution were used and reactions were run in parallel. Crude reaction mixtures were purified by reverse phase chromatography.

| | | MS | |
|---|---|---|---|
| Example | Name and Structure | (ES+) | HRMS |
| 339 | 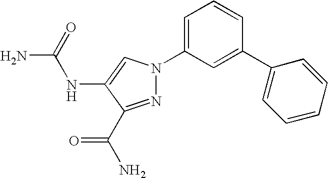 | 322 | 322.1299 |
| | 4-[(aminocarbonyl)amino]-1-(1,1′-biphenyl-3-yl)-1H-pyrazole-3- | | |
| | carboxamide | | |
| 340 | 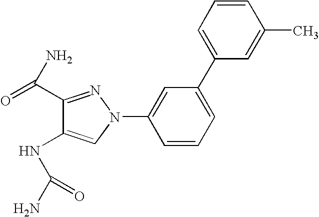 | 336 | 336.1433 |
| | 4-[(aminocarbonyl)amino]-1-(3′-methyl-1,1′-biphenyl-3-yl)-1H- | | |
| | pyrazole-3-carboxamide | | |
| 341 | 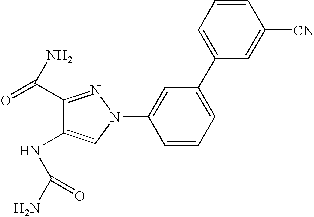 | 347 | 347.1263 |
| | 4-[(aminocarbonyl)amino]-1-(3′-cyano-1,1′-biphenyl-3-yl)-1H- | | |
| | pyrazole-3-carboxamide | | |
| 342 | 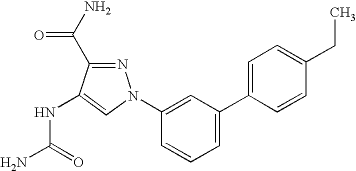 | 350 | 350.1612 |
| | 4-[(aminocarbonyl)amino]-1-(4′-ethyl-1,1′-biphenyl-3-yl)-1H-pyrazole- | | |
| | 3-carboxamide | | |
| 343 | 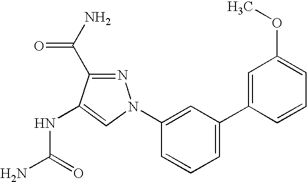 | 352 | 352.1387 |
| | 4-[(aminocarbonyl)amino]-1-(3′-methoxy-1,1′-biphenyl-3-yl)-1H- | | |
| | pyrazole-3-carboxamide | | |
| 344 | 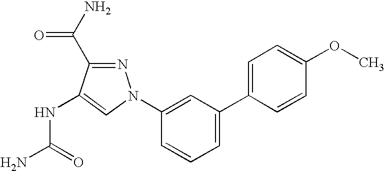 | 352 | 352.1374 |
| | 4-[(aminocarbonyl)amino]-1-(4′-methoxy-1,1′-biphenyl-3-yl)-1H- | | |
| | pyrazole-3-carboxamide | | |
| 345 | 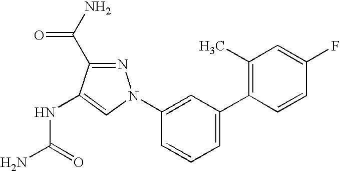 | 354 | 354.1326 |
| | 4-[(aminocarbonyl)amino]-1-(4′-fluoro-2′-methyl-1,1′-biphenyl-3-yl)- | | |
| | 1H-pyrazole-3-carboxamide | | |
| 346 | 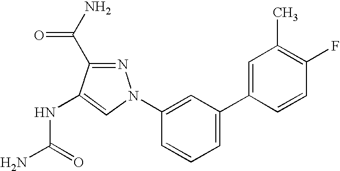 | 354 | 354.1341 |
| | 4-[(aminocarbonyl)amino]-1-(4′-fluoro-3′-methyl-1,1′-biphenyl-3-yl)- | | |
| | 1H-pyrazole-3-carboxamide | | |
| 347 | 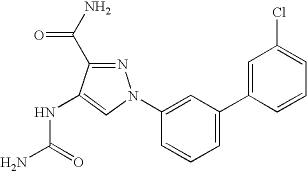 | 356 | 356.0957 |
| | 4-[(aminocarbonyl)amino]-1-(3′-chloro-1,1′-biphenyl-3-yl)-1H- | | |
| | pyrazole-3-carboxamide | | |
| 348 | 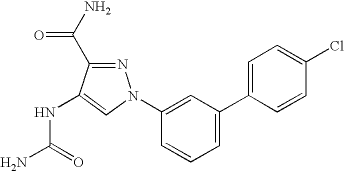 | 356 | 356.0947 |
| | 4-[(aminocarbonyl)amino]-1-(4′-chloro-1,1′-biphenyl-3-yl)-1H- | | |
| | pyrazole-3-carboxamide | | |
| 349 | 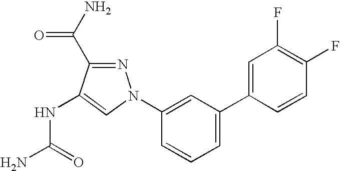 | 358 | 358.1138 |
| | 4-[(aminocarbonyl)amino]-1-(3′,4′-difluoro-1,1′-biphenyl-3-yl)-1H- | | |
| | pyrazole-3-carboxamide | | |
| 350 | 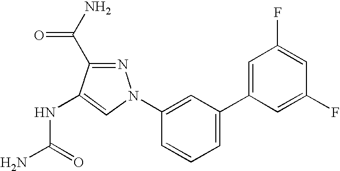 | 358 | 358.1150 |
| | 4-[(aminocarbonyl)amino]-1-(3′,5′-difluoro-1,1′-biphenyl-3-yl)-1H- | | |
| | pyrazole-3-carboxamide | | |
| 351 | 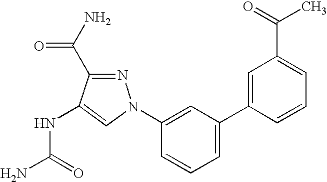 | 364 | 364.1446 |
| | 1-(3′-acetyl-1,1′-biphenyl-3-yl)-4-[(aminocarbonyl)amino]-1H- | | |
| | pyrazole-3-carboxamide | | |
| 352 | 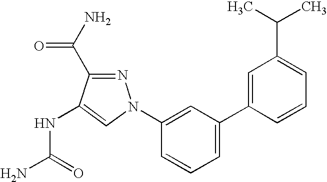 | 364 | 364.1790 |
| | 4-[(aminocarbonyl)amino]-1-(3′-isopropyl-1,1′-biphenyl-3-yl)-1H- | | |
| | pyrazole-3-carboxamide | | |
| 353 | 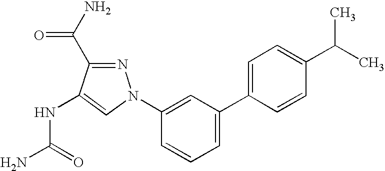 | 364 | 364.1777 |
| | 4-[(aminocarbonyl)amino]-1-(4′-isopropyl-1,1′-biphenyl-3-yl)-1H- | | |
| | pyrazole-3-carboxamide | | |
| 354 | 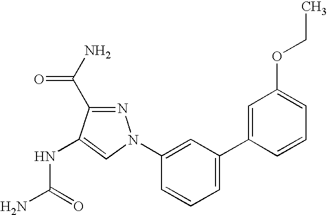 | 366 | 366.1564 |
| | 4-[(aminocarbonyl)amino]-1-(3′-ethoxy-1,1′-biphenyl-3-yl)-1H- | | |
| | pyrazole-3-carboxamide | | |
| 355 | 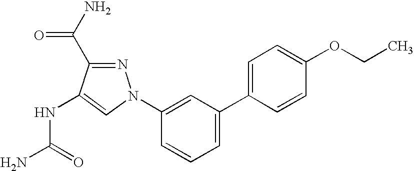 | 366 | 366.1540 |
| | 4-[(aminocarbonyl)amino]-1-(4′-ethoxy-1,1′-biphenyl-3-yl)-1H- | | |
| | pyrazole-3-carboxamide | | |
| 356 | 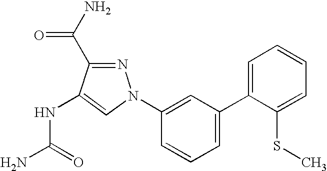 | | 368.1183 |
| | 4-[(aminocarbonyl)amino]-1-[2′-(methylthio)-1,1′-biphenyl-3-yl]-1H- | | |
| | pyrazole-3-carboxamide | | |
| 357 | 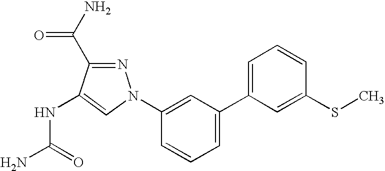 | 368 | 368.1182 |
| | 4-[(aminocarbonyl)amino]-1-[3′-(methylthio)-1,1′-biphenyl-3-yl]-1H- | | |
| | pyrazole-3-carboxamide | | |
| 358 | 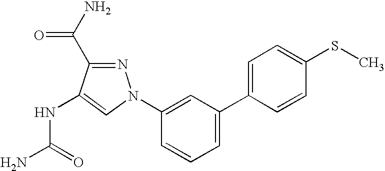 | 368 | 368.1221 |
| | 4-[(aminocarbonyl)amino]-1-[4′-(methylthio)-1,1′-biphenyl-3-yl]-1H- | | |
| | pyrazole-3-carboxamide | | |
| 359 | 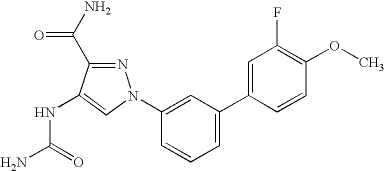 | 370 | 370.1330 |
| | 4-[(aminocarbonyl)amino]-1-(3′-fluoro-4′-methoxy-1,1′-biphenyl-3-yl)- | | |
| | 1H-pyrazole-3-carboxamide | | |
| 360 | 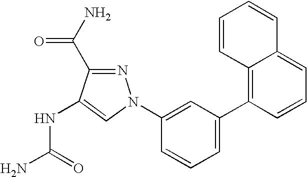 | 372 | 372.1438 |
| | 4-[(aminocarbonyl)amino]-1-[3-(1-naphthyl)phenyl]-1H-pyrazole-3- | | |
| | carboxamide | | |
| 361 | 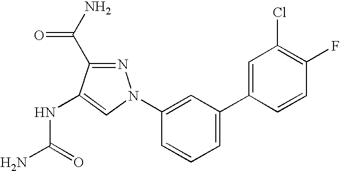 | 374 | 374.0798 |
| | 4-[(aminocarbonyl)amino]-1-(3′-chloro-4′-fluoro-1,1′-biphenyl-3-yl)- | | |
| | 1H-pyrazole-3-carboxamide | | |
| 362 | 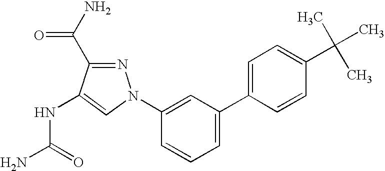 | 378 | 378.1941 |
| | 4-[(aminocarbonyl)amino]-1-(4′-tert-butyl-1,1′-biphenyl-3-yl)-1H- | | |
| | pyrazole-3-carboxamide | | |

### Examples 363-375

Prepared according to the procedure of Examples 339-362, with the following exceptions: 4-[(aminocarbonyl)amino]-1-(3-bromophenyl)-1H-pyrazole-3-carboxamide (Example 20, 0.075 g, 0.231 mmol), Pd[Ph₃]₄ (27 mg, 0.023 mmol), 0.303 mmol of an arylboronic acid, DMF (2 mL) and 0.347 mL of 2M Cs2CO3 solution were used and reactions were run in parallel. Crude reaction mixtures were purified by reverse phase chromatography.

| | | MS | |
|---|---|---|---|
| Example | Name and Structure | (ES+) | HRMS |
| 363 | 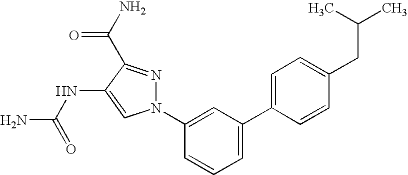 | 378 | 378.1943 |
| | 4-[(aminocarbonyl)amino]-1-(4′-isobutyl-1,1′-biphenyl-3-yl)-1H- | | |
| | pyrazole-3-carboxamide | | |
| 364 | 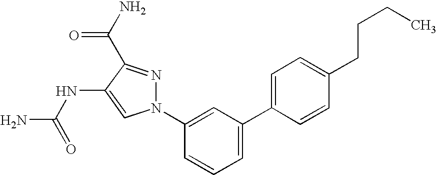 | 378 | 378.1968 |
| | 4-[(aminocarbonyl)amino]-1-(4′-butyl-1,1′-biphenyl-3-yl)-1H-pyrazole- | | |
| | 3-carboxamide | | |
| 365 | 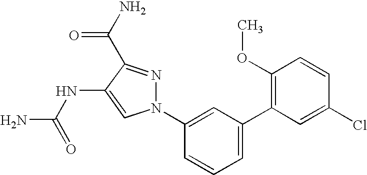 | 386 | 386.0998 |
| | 4-[(aminocarbonyl)amino]-1-(5′-chloro-2′-methoxy-1,1′-biphenyl-3- | | |
| | yl)-1H-pyrazole-3-carboxamide | | |
| 366 | 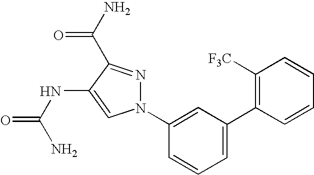 | 390 | 390.1220 |
| | 4-[(aminocarbonyl)amino]-1-[2′-(trifluoromeethyl)-1,1′-biphenyl-3-yl]- | | |
| | 1H-pyrazole-3-carboxamide | | |
| 367 | 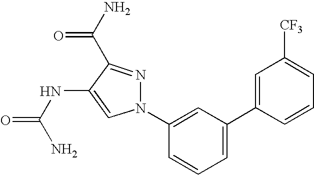 | 390 | 390.1169 |
| | 4-[(aminocarbonyl)amino]-1-[3′-(trifluoromethyl)-1,1′-biphenyl-3-yl]- | | |
| | 1H-pyrazole-3-carboxamide | | |
| 368 | 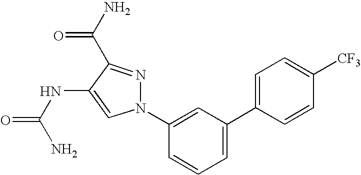 | 390 | 390.1176 |
| | 4-[(aminocarbonyl)amino]-1-[4′-(trifluoromethyl)-1,1′-biphenyl-3-yl]- | | |
| | 1H-pyrazole-3-carboxamide | | |
| 369 | 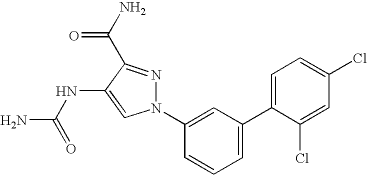 | 390 | 390.0498 |
| | 4-[(aminocarbonyl)amino]-1-(2′,4′-dichloro-1,1′-biphenyl-3-yl)-1H- | | |
| | pyrazole-3-carboxamide | | |
| 370 | 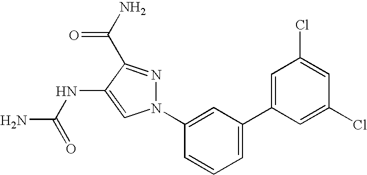 | 390 | 390.0461 |
| | 4-[(aminocarbonyl)amino]-1-(3′,5′-dichloro-1,1′-biphenyl-3-yl)-1H- | | |
| | pyrazole-3-carboxamide | | |
| 371 | 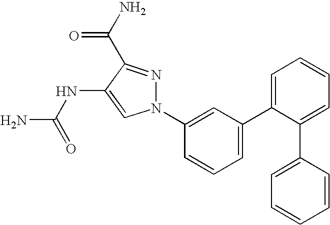 | 398 | 398.1628 |
| | 4-[(aminocarbonyl)amino]-1-(1,1′:2′,1″-terphenyl-3-yl)-1H-pyrazole-3- | | |
| | carboxamide | | |
| 372 | 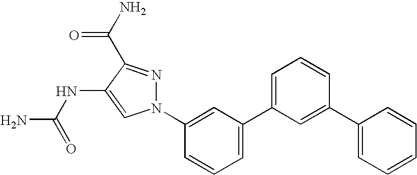 | 398 | 398.1599 |
| | 4-[(aminocarbonyl)amino]-1-(1,1′:4+,1″-terphenyl-3-yl)-1H-pyrazole-3- | | |
| | carboxamide | | |
| 373 | 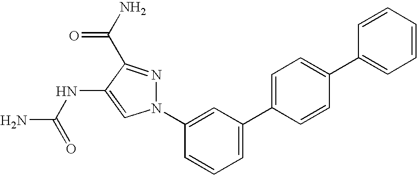 | 398 | 398.1597 |
| | 4-[(aminocarbonyl)amino]-1-(1,1′:4′,1″-terphenyl-3-yl)-1H-pyrazole-3- | | |
| | carboxamide | | |
| 374 | 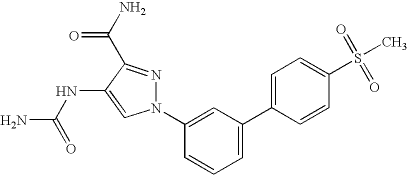 | 400 | 400.1101 |
| | 4-[(aminocarbonyl)amino]-1-[4′-(methylsulfonyl)-1,1′-biphenyl-3-yl]- | | |
| | 1H-pyrazole-3-carboxamide | | |
| 375 | 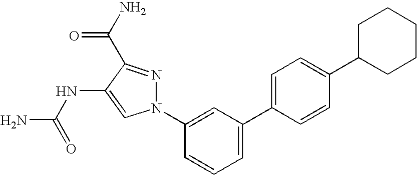 | 404 | 404.2101 |
| | 4-[(aminocarbonyl)amino]-1-(4′-cyclohexyl-1,1′-biphenyl-3-yl)-1H- | | |
| | pyrazole-3-carboxamide | | |

### Example 376

### 1-(1-(4-(4-fluorophenylthio)-2-chlorophenyl)-3-carbamoyl-1H-pyrazol-4-yl)urea

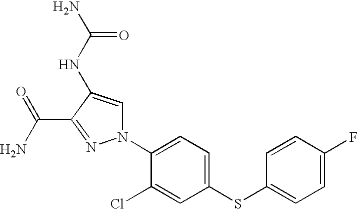

A vial was charged with 4-[(aminocarbonyl)amino]-1-(2-chloro-4-iodophenyl)-1H-pyrazole-3-carboxamide (Example 251, 0.19 g, 0.47 mM). To this was added copper [I] iodide (0.087 g, 0.46 mM) and N,N-diisoproplylethylamine (1 mL, 4.6 mM). Nitrogen was bubbled through the solution as ethylene glycol (0.24 mL, 4.3 mM), 4-fluorobenzenethiol (0.1 mL, 0.9 mM) and DMF (1.5 mL) were added. The vial was tightly capped and heated to 105° C. for 4 h. The reaction was added to dilute aqueous HCl and filtered. It was then washed with dilute sodium hydroxide solution and water. HPLC of the yellow solid showed desired product plus a 6% by-product which was more polar. In order to remove trace copper residue, the crude product was dissolved in pyridine and exposed to air for 10-15 minutes, then dilute ammonium hydroxide (about 25%) was added. The solid was filtered and washed with water. ¹H NMR: (300 MHz, DMSO-d₆) δ 6.47 (bs, 2H); 7.10-7.76 (m, 11H); 8.24 (s, 1H); 8.66 (s, 1H). LCMS (ES I—) for C₁₇H₁₃Cl₁F₁NO₂S₁ m/z 403.9 (M−H)⁻.

### Example 377

### 1-(1-(4-(4-fluorophenylsulfonyl)-2-chlorophenyl)-3-carbamoyl-1H-pyrazol-4-yl)urea

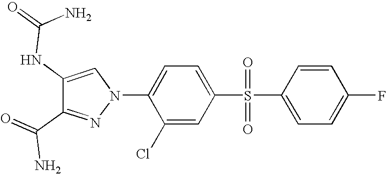

1-(1-(4-(4-fluorophenylthio)-2-chlorophenyl)-3-carbamoyl-1H-pyrazol-4-yl)urea (Example 376, 0.103 g, 0.26 mM) was treated with 0.256 g of 77% 3-chloroperoxybenzoic acid and 10 mL methylene chloride. The reaction was stirred at room temperature over the weekend. The reaction was not complete. It was treated with 6 mL THF and 175 mg more MCPBA and stirred at room temperature overnight. The crude product was applied to silica gel in a 30 mL scintered glass funnel and eluted with 5, 10, and 20% methanol in methylene chloride. The desired product 3 came off in 10 and 20% methanol-methylene chloride. It was evaporated to a small amount of methanol, whereupon it crystallized. It was then evaporated on high vacuum. A 22% yield was obtained. ¹H NMR: (300 MHz, DMSO-d₆) δ 6.47 (bs, 2H); 7.50-7.60 (m, 4H); 7.72 (bs, 1H); 7.92 (m, 1H); 8.00-8.20 (m, 3H); 8.67 (s, 1H). LCMS (ESI−) for C₁₇H₁₃Cl₁F₁N₅O₄Sl m/z 436.0 (M−H)⁻.

### Example 378

### 1-(1-(4-(4-fluorophenylsulfonyl)-2-fluorophenyl)-3-carbamoyl-1H-pyrazol-4-yl)urea

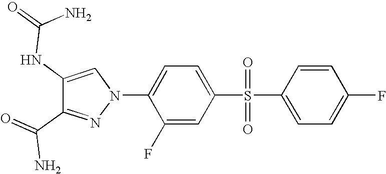

1-(1-(4-(4-fluorophenylthio)-2-fluorophenyl)-3-carbamoyl-1H-pyrazol-4-yl)urea (0.130 g, 0.33 mM) was treated with 0.545 g of 77% 3-chloroperoxybenzoic acid and 8 mL methylene chloride. The reaction was stirred at room temperature overnight. The reaction was diluted with methylene chloride and extracted with saturated sodium carbonate 3 times. The methylene chloride layer was washed with brine, dried and evaporated. The crude product weighed 111 mg. The water layer containing a little methylene chloride and some emulsion was concentrated partially and filtered. Approximately 50 mg of a solid containing more product was obtained. The product was purified by chromatography on silica gel in methanol-methylene chloride mixtures. The fractions were evaporated, and resuspended in methanol-methylene chloride and filtered. The solvent was partially evaporated to precipitate/crystallize the product. The desired product was filtered and dried. A 22% yield was obtained. ¹H NMR: (300 MHz, DMSO-d₆) δ 6.53 (bs, 2H); 7.40-7.60 (m, 2H); 7.63 (s, 1H); 7.80-8.00 (m, 2H); 8.00-8.15 (m, 4H); 8.53 (m, 1H); 8.66 (s, 1H). LCMS (ESI−) for C₁₇H₁₃F₂N₅O₄S₁ m/z 420.0 (M−H)⁻. ¹³C NMR(75 MHz, DMSO-d₆) δ 117.67, 117.98, 121.3, 125.19, 125.57, 127.37, 131.57, 131.71, 135.45, 137.4, 140.8, 151.2, 154.8, 155.96, 164.3, 165.06, 167.8.

### Example 379

### 3-(4′-{3-(aminocarbonyl)-4-[(aminocarbonyl)amino]-1H-pyrazol-1-yl}-1,1′-biphenyl-2-yl)propanoic acid

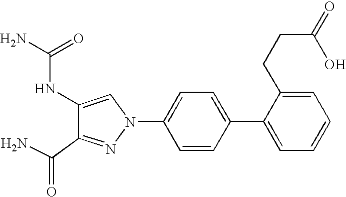

Prepared according to the procedure of Examples 21-95. ¹H NMR DMSO-d₆: δ 2.56 (t, 2H, J=7.4 Hz), 2.88 (t, 2H, J=7.4 Hz), 6.53 (br s, 2H), 7.22 (d, 1H, J=7.6 Hz), 7.36 (t, 1H, J=7.6 Hz), 7.48-7.55 (m, 2H), 7.56 (br s, 1H), 7.74-7.86 (m, 3H), 7.95 (d, 2H, J=8.8 Hz), 8.62 (s, 1H), 8.69 (s, 1H). Mass of Molecular Ion: 394.

Examples 380-382 were prepared according to the procedure of Examples 143-164.

| | | | Mass of |
|---|---|---|---|
| | | | Molecular |
| Example | Name and Structure | ¹H NMR | Ion |
| 380 | 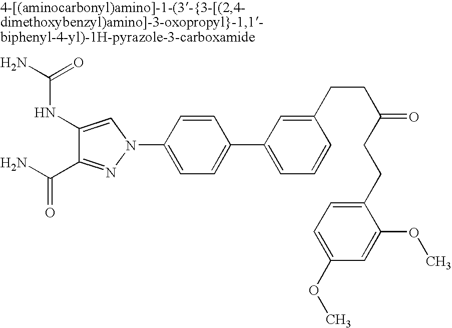 | (CD₃OD): δ 2.58(t, 2H, J=7.5 Hz), 3.02(t, 2H, J=7.4 Hz), 3.65(s, 3H), 3.71(s, 3H), 4.22(s, 2H), 6.29(dd, 1H, J=10.6, J=2.3 Hz), 6.44 (d, 1H, J=2.2 Hz), 6.87 (d, 1H, J=8.4 Hz), 7.21 (d, 1H, J=7.4 Hz), 7.35 (t, 1H, J=8.3 Hz), 7.46-7.55(m, 2H), 7.72(d, 2H, J=8.7 Hz), 7.95(d, 2H, J=8.7 Hz), 8.60(s, 1H) | 543 |
| 381 | 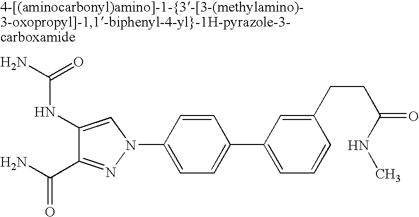 | DMSO-d₆ δ 2.42(t, 2H, J=7.3 Hz), 2.56(d, 3H, J=4.6 Hz), 2.88(t, 2H, J=7.3 Hz), 6.56(br s, 2H0, 7.20(d, 1H, J=7.5 Hz), 7.37(t, 1H, J=7.5 Hz), 7.49-7.57(m, 3H), 7.72-7.83(m, 4H0, 7.96 (d, 2H, J=8.8 Hz), 8.62 (s, 1H0, 8.71(s, 1H) | 407 |
| 382 | 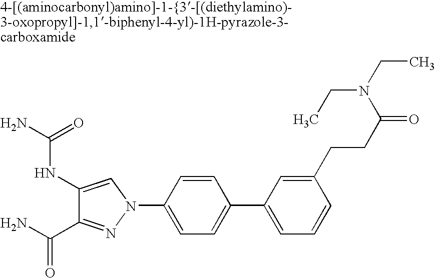 | (CD₃OD): δ 1.02-1.14 (m, 6H), 2.72(t, 2H, J=7.4 Hz), 3.04(t, 2H, J=7.4 Hz), 3.22-3.40(m, 4H0, 7.25(d, 1H, J=7.6), 7.37(t, 1H, J=7.6 Hz), 7.48-7.58(m, 3H), 7.76 (d, 2H, J=8.8 Hz), 7.90 (d, 2H, J=8.8 Hz), 8.56 (s, 1H) | 449 |

### Example 383

### 4-[(aminocarbonyl)amino]-1-{3′-[2-(methylamino)-2-oxoethoxy]-1,1′-biphenyl-4-yl}-1H-pyrazole-3-carboxamide

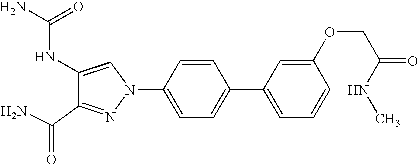

Prepared according to the procedure of Examples 252-257. ¹H NMR (DMSO-d₆) δ 2.63 (d, 3H, J=4.6 Hz), 4.55 (s, 2H), 6.52 (br s, 2H), 6.98 (d, 1H, J=9.0 Hz), 7.28-7.48 (m, 3H), 7.52 (br s, 1H), 7.75-7.85 (m, 3H), 8.00-8.12 (m, 1H), 7.95 (d, 2H, J=8.8 Hz), 8.62 (s, 1H), 8.70 (s, 1H). Mass of Molecular Ion: 409.

### Example 384

### Human IKK-2 Enzyme Assay

Materials: SAM²™96 Biotin capture plates were from Promega. Anti-FLAG affinity resin, FLAG-peptide, NP-40 (Nonidet P-40), BSA, ATP, ADP, AMP, LPS (E. coli serotype 0111:B4), and dithiothreitol were obtained from Sigma Chemicals. Antibodies specific for NEMO (IKK-γ) (FL-419), IKK-1 (H-744), IKK-2(H-470) and IκBa(C-21) were purchased from Santa Cruz Biotechnology. Ni-NTA resin was purchased from Qiagen. Peptides were purchased from American Peptide Company. Protease inhibitor cocktail tablets were from Boehringer Mannheim. Sephacryl S-300 column was from Pharmacia LKB Biotechnology. Centriprep-10 concentrators with a molecular weight cutoff of 10 kDa and membranes with molecular weight cut-off of 30 kDa were obtained from Amicon. [γ-³³P] ATP (2500 Ci/mmol) and [γ-³²P] ATP (6000 Ci/mmol) were purchased from Amersham. The other reagents used were of the highest grade commercially available.

Cloning and Expression: cDNAs of human IKK-1 and IKK-2 were amplified by reverse transcriptase-polymerase chain reaction from human placental RNA (Clonetech). hlKK-1 was subcloned into pFastBac HTa (Life Technologies) and expressed as N-terminal His₆-tagged fusion protein. The hlKK-2 cDNA was amplified using a reverse oligonucleotide primer which incorporated the peptide sequence for a FLAG-epitope tag at the C-terminus of the IKK-2 coding region (DYKDDDDKD). The hlKK-2:FLAG cDNA was subcloned into the baculovirus vector pFastBac. The rhIKK-2 (Si77S, E177E) mutant was constructed in the same vector used for wild type rhIKK-2 using a QuikChange™ mutagenesis kit (Stratagene). Viral stocks of each construct were used to infect insect cells grown in 40 L suspension culture. The cells were lysed at a time that maximal expression and rhIKK activity were demonstrated. Cell lysates were stored at −80° C. until purification of the recombinant proteins was undertaken as described below.

Enzyme Isolation: All purification procedures were carried out at 4° C. unless otherwise noted. Buffers used are: buffer A: 20 mM Tris-HCl, pH 7.6, containing 50 mM NaCl, 20 mM NaF, 20 mM β-Glycerophosphate, 500 μM sodium orthovanadate, 2.5 mM metabisulfite, 5 mM benzamidine, 1 mM EDTA, 0.5 mM EGTA, 10% glycerol, 1 mM DTT, 1× Complete™ protease inhibitors; buffer B: same as buffer A, except 150 mM NaCl, and buffer C: same as buffer A, except 500 mM NaCl.

Isolation of rhIKK-1 homodimer: Cells from an 8-liter fermentation of baculovirus-expressed IKK-1 tagged with His peptide were centrifuged and the cell pellet (MOI 0.1, I=72 h) was re-suspended in 100 mL of buffer C. The cells were microfluidized and centrifuged at 100,000×g for 45 min. The supernatant was collected, imidazole added to the final concentration of 10 mM and incubated with 25 mL of Ni-NTA resin for 2 h. The suspension was poured into a 25 mL column and washed with 250 mL of buffer C and then with 125 mL of 50 mM imidazole in buffer C. rhIKK-1 homodimer was eluted using 300 mM imidazole in buffer C. BSA and NP-40 were added to the enzyme fractions to the final concentration of 0.1%. The enzyme was dialyzed against buffer B, aliquoted and stored at −80° C.

Isolation of rhIKK-2 homodimer: A 10-liter culture of baculovirus-expressing IKK-2 tagged with FLAG peptide was centrifuged and the cell pellet (MOI=0.1 and 1=72 h) was re-suspended in buffer A. These cells were microfluidized, and centrifuged at 100,000×g for 45 min. Supernatant was passed over a G-25 column equilibrated with Buffer A. Protein peak was collected and incubated with anti-FLAG affinity resin on a rotator overnight in buffer B. The resin was washed in batch with 10-15 bed volumes of buffer C. Washed resin was poured into a column and rhIKK-2 homodimer was eluted using 5 bed volumes of buffer B containing FLAG peptide. 5 mM DTT, 0.1% NP-40 and BSA (concentrated to 0.1% in final amount) was added to the eluted enzyme before concentrating in using an Amicon membrane with a molecular weight cut-off of 30 kDa. Enzyme was aliquoted and stored at −80° C.

Isolation of rhIKK-1/IKK-2 heterodimer: The heterodimer enzyme was produced by coinfection in a baculovirus system (FLAG IKK-2/IKK-1 His; MOI=0.1 and 1=72 h). Infected cells were centrifuged and the cell pellet (10.0 g) was suspended in 50 mL of buffer A. The protein suspension was microfluidized and centrifuged at 100,000×g for 45 min. Imidazole was added to the supernatant to a final concentration of 10 mM. The protein was allowed to bind 25 mL of Ni-NTA resin by mixing for 2 h. The protein-resin slurry was poured into a 25 mL column and washed with 250 mL of buffer A containing 10 mM imidazole followed by 125 mL of buffer A containing 50 mM imidazole. Buffer A, containing 300 mM imidazole, was then used to elute the protein. A 75 mL pool was collected and NP-40 was added to a final concentration of 0.1%. The protein solution was then dialyzed against buffer B. The dialyzed heterodimer enzyme was then allowed to bind to 25 mL of anti-FLAG M2 agarose affinity gel overnight with constant mixing. The protein-resin slurry was then centrifuged for 5 min at 2,000 rpm. The supernatant was collected and the resin re-suspended in 100 mL of buffer C containing 0.1% NP-40. The resin was washed with 375 mL of buffer C containing 0.1% NP-40. The protein-resin was poured into a 25 mL column and the enzyme eluted using buffer B containing FLAG peptide. Enzyme fractions (100 mL) were collected and concentrated to 20 mL using an Amicon membrane with molecular weight cut-off of 30 kDa. Bovine serum albumin was added to the concentrated enzyme to final concentration of 0.1%. The enzyme was then aliquoted and stored at −80° C.

Cell Culture: The wild type (wt) human pre-B cell line, 70Z/3, and its mutant, 1.3E2, were generously provided by Dr. Carol Sibley. Wt 70Z/3 and 1.3E2 cells were grown in RPMI 1640 (Gibco) supplemented with 7% defined bovine serum (Hyclone) and 50 μM 2-mercaptoethanol. Human monocytic leukemia THP-1 cells, obtained from ATCC, were cultured in RPMI 1640 supplemented with 10% defined bovine serum, 10 mM HEPES, 1.0 mM sodium pyruvate and 50 μM 2-mercaptoethanol. For experiments, cells were plated in 6 well plates at 1×10⁶ cells/mL in fresh media. Pre-B cells were stimulated by the addition of 10 μg/mL LPS for varying lengths of time ranging from 0-4 h. THP-1 cells were stimulated by the addition of 1 μg/mL LPS for 45 min. Cells were pelleted, washed with cold 50 mM sodium phosphate buffer, pH 7.4 containing 0.15 M NaCl and lysed at 4° C. in 20 mM Hepes buffer, pH 7.6 containing 50 mM NaCl, 1 mM EDTA, 1 mM EGTA, 1 mM sodium orthovanadate, 10 mM β-glycerophosphate, 1 mM NaF, 1 mM PMSF, 1 mM DTT and 0.5% NP40 (lysis buffer). The cytosolic fractions obtained following centrifugation at 10,000×g were stored at −80° C. until used.

Immunoprecipitation and Western Blotting: SF9 cells paste containing rhIKKs were centrifuged (100,000×g, 10 min) to remove debris. rhIKKs were immunoprecipitated (100 μg of cell paste) from the cell supernatant using 3 μg of anti-NEMO antibody (FL-419), followed by coupling to protein A sepharose beads. rhIKKs were also immunoprecipitated from affinity chromatography purified protein preparations (1 μg) using anti-FLAG, anti-His or anti-NEMO antibodies (1-4 μg) followed by protein A sepharose coupling. The native, human IKK complex was immunoprecipitated from THP-1 cell homogenates (300 μg/condition) using the anti-NEMO antibody. Immune complexes were pelleted and washed 3 times with 1 mL cold lysis buffer. Immunoprecipitated rhIKKs were chromatographed by SDS-PAGE (8% Tris-glycine) and transferred to nitrocellulose membranes (Novex) and detected by chemiluminescense (SuperSignal) using specific anti-IKK antibodies (IKK-2H-470, IKK-1H-744). Native IKK-2, IκBA, and NEMO proteins from cytosolic lysates (20-80 μg) were separated by SDS-PAGE and visualized by chemiluminescense using specific antibodies.

Phosphatase Treatment: Immunoprecipitated rhIKKs were washed 2 times in 50 mM Tris-HCl, pH 8.2 containing 0.1 mM EDTA, 1 mM DTT, 1 mM PMSF and 2 mM MnCl₂ and resuspended in 50 μL. Phosphatase (APPase, 1000U) was pre-diluted in the same buffer and added to the IKK samples. Following incubation at rt for 30 min with intermittent mixing, cold lysis buffer was added to the tubes to stop the reaction. After several washes, 10% of the beads were removed for Western analysis, and the remaining material was pelleted and resuspended in 100 μL of the buffer used for the in vitro kinase assay.

IKK-1 SAM Enzyme Assay: IKK-1 kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu), a SAM²™96 Biotin capture plate and a vacuum system. The standard reaction mixture contained 5 μM biotinylated IκBα peptide, 1 μM [γ-³³P] ATP (about 1×10⁵ cpm), 1 mM DTT, 50 mM KCl, 2 mM MgCl₂, 2 mM MnCl₂, 10 mM NaF, 25 mM Hepes buffer, pH. 7.6 and enzyme solution (1-10 μL) in a final volume of 50 μL. After incubation at 25° C. for 30 min, 25 μL of the reaction mixture was withdrawn and added to a SAM²™96 Biotin capture 96-well plate. Each well was then washed successively with 800 μL 2 M NaCl, 1.2 mL of NaCl containing 1% H₃PO₄, 400 μL H₂O, and 200 μL 95% ethanol. The plate was allowed to dry in a hood at 25° C. for 1 h and then 25 μL of scintillation fluid (Microscint 20) was added to each well. Incorporation of [γ-³³P] ATP was measured using a Top-Count NXT (Packard). Under each assay condition, the degree of phosphorylation of IκBα peptide substrate was linear with time and concentration for all purified enzymes. Results from the biotinylated peptide assay were confirmed by SDS-PAGE analysis of kinase reaction utilizing a GST-IκBa, ₅₄ and [γ-³²P] ATP. The resulting radiolabeled substrate was quantitated by Phosphoimager (Molecular Dynamics). An ion exchange resin assay was also employed using [γ-³³P] ATP and GST-IκBα₁₋₅₄ fusion protein as the substrates. Each assay system yielded consistent results in regard to Kₘ and specific activities for each of the purified kinase isoforms. One unit of enzyme activity was defined as the amount required to catalyze the transfer of 1 nmole of phosphate from ATP to IκBA peptide per min. Specific activity was expressed as units per mg of protein. For experiments related to Kₘ determination of purified enzymes, various concentrations of ATP or IκBα peptide were used in the assay at either a fixed IκBα or ATP concentration. For IκBα peptide Kₘ, assays were carried out with 0.1 μg of enzyme, 5 μM ATP and IκBα peptide from 0.5 to 20 μM. For ATP Km, assays were carried out with 0.1 μg of enzyme, 10 PM IκBα peptide and ATP from 0.1 to 10 μM. For Km determination of rhIKK-1 homodimer, due to its low activity and higher Kₘ for IκBA peptide, rhIKK-1 homodimer (0.3 μg) was assayed with 125 μM IκBα peptide and a 5-fold higher specific activity of ATP (from 0.1 to 10 μM) for ATP Kₘ experiments and a 5-fold higher specific activity of 5 μM ATP and IκBα peptide (from 5 to 200 μM) for IκBA peptide Kₘ experiments.

IKK heterodimer Resin Enzyme Assay: IKK heterodimer kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu) (American Peptide Co.). 20 μL of the standard reaction mixture contained 5 μM biotinylated IκBα peptide, 0.1 μCi/reaction [γ-³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH 7.6 and 20 μL enzyme solution and 10 μl inhibitor in a final volume of 50 μL. After incubation at 25° C. for 30 min, 150 μL resin (Dowex anion-exchange resin AG1X8 200-400 mesh) in 900 mM formate, pH 3.0 was added to each well to stop the reaction. Resin was allowed to settle for 1 h and 50 μL of supernatant was removed to a Micolite-2 flat bottom plate (Dynex). 150 μL of scintillation fluid (Microscint 40) (Packard) was added to each well. Incorporation of [γ-³³P] ATP was measured using a Top-Count NXT (Packard).

IKK-2 Resin Enzyme Assay: IKK-2 kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu) (American Peptide Co.). 20 μL of the standard reaction mixture contained 5 μM biotinylated IκBα peptide, 0.1 μCi/reaction [γ-³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH 7.6 and 20 μL enzyme solution and 10 μL inhibitor in a final volume of 50 μL. After incubation at 25° C. for 30 min, 150 μL resin (Dowex anion-exchange resin AG1X8 200-400 mesh) in 900 mM formate, pH 3.0 was added to each well to stop the reaction. Resin was allowed to settle for 1 h and 50 μL of supernatant was removed to a Micolite-2 flat bottom plate (Dynex). 150 μL of scintillation fluid (Microscint 40) (Packard) was added to each well. Incorporation of [γ-³³P] ATP was measured using a Top-Count NXT (Packard).

IKK-2 IC₅₀ values obtained from the assay described above are shown below (Example 385).

### Example 385

### Rat IKK-2 enzyme assay

Kinase activity was measured using a biotinylated IκBβ peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu) (American Peptide Co.). 20 μL of the standard reaction mixture contained 5 μM biotinylated IκBα peptide, 0.1 μCi/reaction [γ-³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH. 7.6 and 20 μL enzyme solution (50 nM/rxn or 200 ng/rxn) and 10 μL inhibitor in a final volume of 50 μL. After incubation at 25° C. for 60 min, 150 μL resin was added to each well to stop the reaction. Resin was mixed and allowed to settle for 1 h and 50 μL of supernatant was removed to a Micolite 2 flat bottom plate (Dynex). 150 μL of scintillation fluid (Microscint 40) (Packard) was added to each well. Incorporation of [γ-³³P] ATP was measured using a Top-Count NXT (Packard).

IKK-2 IC₅₀ values obtained from the assay described above are shown in the following table.

| | |
|---|---|
| Ex. | IC₅₀ (μM) |

| No. | hIKK-2 | rIKK-2 |
|---|---|---|
| 1 | 0.307 | 0.108 |
| 2 | 10.7 | |
| 3 | 1.35 | 0.787 |
| 4 | 2.87 | 1.933 |
| 5 | 5.09 | 3.710 |
| 6 | 0.546 | |
| 7 | 0.355 | 0.121 |
| 8 | 0.0676 | 0.089 |
| 9 | 1.43 | |
| 10 | 2.91 | |
| 11 | 6.36 | |
| 12 | 1.43 | |
| 13 | 0.38 | |
| 14 | 8.95 | |
| 15 | 8.05 | 4.449 |
| 16 | 3.10 | 2.828 |
| 17 | 1.72 | |
| 18 | 0.30 | |
| 19 | 0.31 | 0.352 |
| 20 | 0.61 | |
| 21 | 2.86 | |
| 22 | 1.20 | |
| 23 | 1.64 | |
| 24 | 7.73 | |
| 25 | 1.55 | |
| 26 | 0.59 | |
| 27 | 1.36 | |
| 28 | 2.79 | |
| 29 | 0.53 | |
| 30 | 3.00 | |
| 31 | 4.79 | |
| 32 | 1.16 | |
| 33 | 0.89 | |
| 34 | 4.21 | |
| 35 | 1.33 | |
| 36 | 1.63 | |
| 37 | 0.79 | |
| 38 | 0.83 | |
| 39 | 1.34 | |
| 40 | 0.351 | |
| 41 | 7.59 | |
| 42 | 9.22 | |
| 43 | 8.54 | |
| 44 | 2.08 | |
| 45 | 0.31 | |
| 46 | 5.20 | |
| 47 | 0.66 | |
| 48 | 1.04 | |
| 49 | 2.48 | |
| 50 | 2.08 | |
| 51 | 5.99 | |
| 52 | 4.86 | |
| 53 | 4.07 | |
| 54 | 1.16 | |
| 55 | 1.04 | |
| 56 | 2.16 | |
| 57 | 0.29 | |
| 58 | 2.81 | |
| 59 | 1.64 | |
| 60 | 3.75 | |
| 61 | 6.21 | |
| 62 | 7.91 | |
| 63 | 0.59 | |
| 64 | 6.08 | |
| 65 | 1.80 | |
| 66 | 5.20 | |
| 67 | 5.24 | |
| 68 | 0.55 | |
| 69 | 0.65 | |
| 70 | 0.88 | |
| 71 | 2.25 | |
| 72 | 0.44 | |
| 73 | 8.03 | |
| 74 | 0.94 | |
| 75 | 0.48 | |
| 76 | 0.90 | |
| 77 | 0.80 | |
| 78 | 0.81 | |
| 79 | 5.67 | |
| 80 | 0.60 | |
| 81 | 1.65 | |
| 82 | 1.19 | |
| 83 | 1.29 | |
| 84 | 2.06 | |
| 85 | 0.58 | |
| 86 | 5.91 | |
| 87 | 1.52 | |
| 88 | 3.20 | |
| 89 | 0.70 | |
| 90 | 2.81 | |
| 91 | 3.45 | |
| 92 | 6.61 | 2.656 |
| 93 | 3.10 | 1.816 |
| 94 | 0.22 | 0.220 |
| 95 | 2.68 | |
| 96 | 1.45 | |
| 97 | 0.14 | 0.180 |
| 98 | 1.04 | 0.668 |
| 99 | 0.75 | 0.555 |
| 100 | 1.15 | 0.171 |
| 101 | 8.07 | 1.885 |
| 102 | 2.82 | |
| 103 | >20 | |
| 104 | 0.677 | |
| 105 | 0.351 | |
| 106 | 1.06 | |
| 107 | 0.651 | |
| 108 | 0.947 | |
| 109 | 5.52 | |
| 110 | 0.743 | |
| 111 | 4.38 | |
| 112 | 1.99 | |
| 113 | 0.355 | |
| 114 | 3.19 | |
| 115 | 5.81 | |
| 116 | 11 | |
| 117 | >20 | |
| 118 | 3.07 | |
| 119 | 0.664 | |
| 120 | 0.972 | |
| 121 | 0.385 | |
| 122 | 6.99 | |
| 123 | >20.0 | |
| 124 | 7.41 | |
| 125 | 3.95 | |
| 126 | >20.0 | |
| 127 | >20.0 | |
| 128 | >20.0 | |
| 129 | 1.82 | |
| 130 | 1.79 | |
| 131 | 3.15 | |
| 132 | 18.8 | |
| 133 | 1.63 | |
| 134 | 3.84 | |
| 135 | 1.66 | |
| 136 | >20.0 | |
| 137 | >20.0 | |
| 138 | >20.0 | |
| 139 | 7.82 | |
| 140 | >20.0 | |
| 141 | 11.4 | 3.996 |
| 142 | 0.17 | |
| 143 | 3.31 | |
| 144 | 5.05 | |
| 145 | 0.24 | |
| 146 | 6.28 | |
| 147 | 6.01 | |
| 148 | 7.14 | |
| 149 | 0.05 | |
| 150 | 0.63 | |
| 151 | 0.31 | |
| 152 | 0.11 | |
| 153 | 0.32 | |
| 154 | 0.26 | |
| 155 | 0.45 | |
| 156 | 0.61 | |
| 157 | 8.08 | |
| 158 | 4.04 | |
| 159 | 5.97 | |
| 160 | 4.79 | 7.190 |
| 161 | 1.36 | |
| 162 | 13.7 | 13.605 |
| 163 | 16.6 | 17.353 |
| 164 | 11.3 | 7.270 |
| 165 | 5.99 | |
| 166 | 0.38 | |
| 167 | 0.50 | |
| 168 | 1.99 | |
| 169 | 1.83 | |
| 170 | 1.58 | |
| 171 | 6.84 | |
| 172 | 0.044 | 0.033 |
| 173 | 0.15 | 0.105 |
| 174 | 3.67 | 2.488 |
| 175 | 0.18 | 0.181 |
| 176 | 0.18 | |
| 177 | 1.02 | |
| 178 | 1.99 | |
| 179 | 0.510 | |
| 180 | 0.228 | |
| 181 | 0.105 | |
| 182 | 11.2 | |
| 183 | 0.13 | |
| 184 | 5.49 | |
| 185 | 5.88 | |
| 186 | 0.86 | 0.475 |
| 187 | 0.821 | 0.441 |
| 188 | 0.46 | |
| 189 | 1.04 | |
| 190 | 0.831 | |
| 191 | 2.54 | |
| 192 | 0.237 | 0.297 |
| 193 | 3.75 | |
| 194 | 0.150 | 0.173 |
| 195 | 0.387 | |
| 196 | 0.718 | |
| 197 | 0.561 | |
| 198 | 6.52 | |
| 199 | 6.56 | |
| 200 | 0.326 | |
| 201 | 0.532 | |
| 202 | 5.97 | |
| 203 | 0.153 | |
| 204 | 0.013 | 0.023 |
| 205 | 1.13 | 0.258 |
| 206 | 0.0514 | |
| 207 | 1.32 | |
| 208 | 0.328 | |
| 209 | 1.04 | 1.078 |
| 210 | 0.0947 | |
| 211 | 10.5 | |
| 212 | 0.844 | |
| 213 | 3.91 | |
| 214 | 0.382 | |
| 215 | 0.322 | 0.091 |
| 216 | 0.159 | |
| 217 | 0.383 | |
| 218 | 1.31 | |
| 219 | 0.175 | |
| 220 | 1.57 | |
| 221 | 2.60 | |
| 222 | >20 | |
| 223 | 1.15 | |
| 224 | 0.327 | |
| 225 | 0.378 | 0.292 |
| 226 | 1.06 | |
| 227 | 1.53 | |
| 228 | >20.0 | |
| 229 | 2.19 | |
| 230 | 2.02 | |
| 231 | 4.88 | |
| 232 | 10.3 | |
| 233 | 0.375 | |
| 234 | 1.71 | |
| 235 | 13.2 | |
| 236 | 3.05 | |
| 237 | 3.90 | |
| 238 | 1.11 | |
| 239 | 0.161 | |
| 240 | 2.02 | |
| 241 | 1.68 | |
| 242 | 1.35 | |
| 243 | 8.11 | |
| 244 | 0.866 | |
| 245 | 5.51 | |
| 246 | 3.30 | |
| 247 | 0.457 | 0.249 |
| 248 | 0.386 | |
| 249 | 0.892 | |
| 250 | 0.279 | |
| 251 | 0.662 | |
| 252 | 0.492 | |
| 253 | 1.01 | |
| 254 | 0.352 | |
| 255 | 0.762 | |
| 256 | 1.07 | |
| 257 | 1.35 | |
| 258 | 1.02 | |
| 259 | 4.01 | |
| 260 | 0.512 | |
| 261 | 1.18 | |
| 262 | 0.842 | |
| 263 | | 0.482 |
| 264 | | 0.170 |
| 265 | | 6.16 |
| 266 | | 0.392 |
| 267 | | 2.75 |
| 268 | | 0.175 |
| 269 | | 0.781 |
| 270 | 3.70 | |
| 271 | | 0.956 |
| 272 | | 0.167 |
| 273 | | 0.203 |
| 274 | >20 | |
| 275 | | 10.8 |
| 276 | | 0.194 |
| 277 | 7.59 | |
| 281 | 10.6 | |
| 282 | 12.6 | |
| 283 | 0.431 | |
| 284 | 0.986 | |
| 285 | 0.578 | |
| 286 | 0.663 | |
| 287 | 0.187 | |
| 288 | 0.252 | |
| 289 | 10.6 | |
| 290 | 4.56 | |
| 291 | 6.23 | |
| 292 | | 4.16 |
| 293 | | 4.82 |
| 294 | | 0.237 |
| 295 | | 0.0369 |
| 296 | 0.656 | |
| 297 | 1.94 | |
| 298 | 0.724 | |
| 299 | 0.595 | |
| 300 | | 0.378 |
| 301 | 0.123 | |
| 302 | 0.229 | |
| 303 | 1.4 | |
| 304 | | 0.547 |
| 305 | | 0.098 |
| 306 | | 0.766 |
| 307 | | 6.44 |
| 308 | | 0.099 |
| 309 | | 0.086 |
| 310 | | 0.095 |
| 311 | | 2.33 |
| 312 | | 0.975 |
| 314 | | >20 |
| 315 | | 0.632 |
| 316 | | 0.533 |
| 317 | | 1.46 |
| 318 | | 8.52 |
| 322 | 2.19 | |
| 324 | | >20 |
| 326 | | 0.365 |
| 328 | | 0.446 |
| 330 | | 1.78 |
| 332 | | 1.08 |
| 334 | | 2.42 |
| 335 | 0.21 | |
| 336 | 0.42 | |
| 337 | 0.75 | |
| 338 | 0.62 | |
| 339 | >20.0 | |
| 340 | >20.0 | |
| 341 | 0.984 | |
| 342 | >20.0 | |
| 343 | 5.18 | |
| 344 | >20.0 | |
| 345 | >20.0 | |
| 346 | >20.0 | |
| 347 | >20.0 | |
| 348 | >20.0 | |
| 349 | >20.0 | |
| 350 | >20.0 | |
| 351 | >20.0 | |
| 352 | >20.0 | |
| 353 | >20.0 | |
| 354 | >20.0 | |
| 355 | 6.15 | |
| 356 | >20.0 | |
| 357 | >20.0 | |
| 358 | 8.57 | |
| 359 | 1.18 | |
| 360 | >20.0 | |
| 361 | >20.0 | |
| 362 | >20.0 | |
| 363 | >20.0 | |
| 364 | 16.2 | |
| 365 | 11.6 | |
| 366 | >20.0 | |
| 367 | >20.0 | |
| 368 | >20 | |
| 369 | 16.7 | |
| 370 | >20.0 | |
| 371 | 1.09 | |
| 372 | >20.0 | |
| 373 | >20.0 | |
| 374 | 0.544 | |
| 375 | >20.0 | |
| 376 | 0.31 | |
| 377 | 0.113 | |
| 378 | 0.315 | |
| 379 | 1.95 | |
| 380 | 1.54 | |
| 381 | 0.42 | |
| 382 | 0.90 | |
| 383 | 0.07 | |

1. A compound of Formula I: 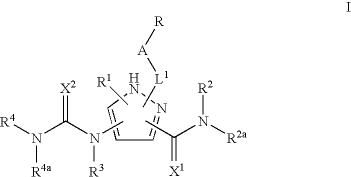
wherein X¹ is selected from the group consisting of O, S, and NR^{6a};
wherein X² is selected from the group consisting of O, S, and NR^{6b};
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)OR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═)OR^{13a}, —NR^{13a}SO₂R^{14a}, —NR^{13a}SO₂NR^{14a}R^{14b}, —NR^{13a}C(═O)NR^{14a}R^{14b}, C(═O)R^{13a}, and —C(═O)NR^{12a}R^{12b};
wherein R is selected from the group consisting of hydrido and -L²R⁵;
wherein L¹ and L² are independently selected from the group consisting of a bond, —O—, —S(═O)ᵣ—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, alkylene, alkenylene, and alkynylene, wherein said R⁷, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, (CH)OR^{8a}, —(CH₂)ₑNR^{8a}R^{8b}, and —(CH₂)ₑS(═O)_{f}R¹¹, or R¹ together with R^{4a} and the atoms to which they are attached form a heterocyclic ring moiety having the structure: 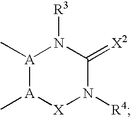
wherein R², R^{2a}, and R³ are independently selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁴, is selected from the group consisting of hydrido, hydroxyl, alkoxy, alkyl, haloalkyl, aryl, and heteroaryl, or R^{4a} together with R¹ and the atoms to which they are attached form a heterocyclic ring having the structure: 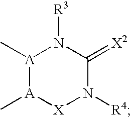
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl, and wherein R⁴, when R¹ together with R^{4a} and the atoms to which they are attached form a heterocyclic ring having 6 or more members, may form a double bond between the nitrogen to which R⁴ is attached and X;
wherein -A-A- represents any two adjacent atoms of the pyrazole ring in Formula I;
wherein X is selected from the group consisting of a bond, —(CHR^{9a})ₓ(CHR^{9b})_{y}(CHR^{c})_{z}—, —NR^{9a}—, —CR^{a} ═CR^{9b}—(CHR^{9c})_{g}, —(CH₂)ₕO—, —(CH₂)ₕS—, —NR^{9a}—NR^{9b}, —N═N—, —O—NR⁹, —S—NR^{9a}, —CH═CH—CH₂—, —CH═CH—NR^{9a}, —CH₂—O—CH₂—, —CH₂—S—CH₂—, —CH₂—N═N—, —NR^{9a}—O—CH₂—, —CH₂—NR⁹a—S—, and —NR^{9a}—S—CH₂—;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, SO₂NR^{12a}R^{12b}, —NR^{13a}R^{13b}, —NR^{12a}C(═O)R^{13a}, —NR¹²C(═O)OR^{13a}, —NR^{13a}SO₂R^{14a}, —NR^{13a}SO₂NR^{14a}R^{14b}, —NR^{13a}C(═O)NR^{14a}R^{14b} C(═O)R^{13a} and C(═O)NR^{12a}R^{12b};
wherein R^{6a}, R^{6b}, R⁷, R^{8a}, R^{8b}, R^{9a}, R^{b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, N, and NR^{13a};
wherein a, d, and e are independently selected from the group consisting of 0, 1, 2, 3, 4, 5, 6, 7, or 8;
wherein b, c, f, and h are independently selected from the group consising of 0, 1, or 2;
wherein g is 0 or 1;
wherein x, y, and z are independently 0, 1, 2, or 3, and wherein x+y+z≦4;
or a pharmaceutically-acceptable salt thereof.

2. A compound according to claim 1, wherein the compound of Formula I is a compound of Formula II: 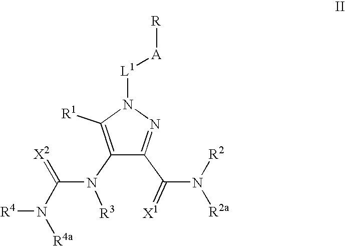
wherein X¹, X², L¹, L², A, R, R¹, R², R^{2a}, R³, R⁴, and R^{4a} are as defined in claim 1.

3. A compound according to claim 2:
wherein A is selected from the group consisting of C₃₋₁₂ cycloalkyl, C₃₋₁₂ cycloalkenyl, 3- to 12-membered heterocycloalkyl, 3- to 12-membered heterocycloalkenyl, C₅₋₁₂ aryl, and 5- to 12-membered heteroaryl, wherein A may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(=°)OR^{13a}, —NR^{13a}So ₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ and L² are independently selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, C₁₋₆ alkylene, C₂₋₆ alkenylene, and C₂₋₆ alkynylene, wherein said R⁷, alkylene, alkenylene, and alkynylene moieties may be substituted by one or more substituents independently selected from R¹⁵;
wherein R¹ is selected from the group consisting of hydrido, cyano, C₁₋₆ alkyl, C₂₋₆ alkenyl, halo, C₁₋₆ haloalkyl, C₁₋₆ hydroxyalkyl, cyano(C₁₋₆ alkyl), C₅₋₁₂ aryl, 5- to 12-membered heteroaryl, —OR^{8aa}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, —(CH₂)ₑOR^{8a}, —(CH₂)ₑNR^{8a}R^{b}, and (CH₂)ₑS(═O)_{f}R¹¹, or R¹ together with R^{4a} and the atoms to which they are attached form a 5- to 8-membered heterocyclic ring moiety having the structure: 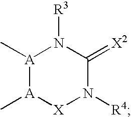
wherein R², R^{2a}, and R¹³ are independently selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R^{4a} is selected from the group consisting of hydrido, hydroxyl, C₁₋₆ alkoxy, C₁₋₆ alkyl, C₁₋₆ haloalkyl, C₅₋₁₂ aryl, and 5- to 12-membered heteroaryl, or R^{4a} together with R¹ and the atoms to which they are attached form a 5- to 8-membered heterocyclic ring having the structure: 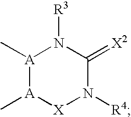
wherein R⁴ is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of C₁₋₆ alkyl, C₃₋₁₂ cycloalkyl, C₃₋₁₂ cycloalkenyl, 3- to 12-membered heterocycloalkyl, 3- to 12-membered heterocycloalkenyl, 5- to 12-membered heteroaryl, and C₅₋₁₂ aryl, wherein R⁵ may be optionally substituted by one or more substituents selected from the group consisting of halo, C₁₋₆ alkylsulfinyl, C₁₋₆ alkylsulfonyl, cyano, C₂, alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂, —NR^{14a}R^{14b}, —NR^{13a}C(═O)NR^{14a}R^{14b}, —C(═O)R^{13a}, and —C(═O)NR^{12a}R^{12b};
wherein R^{6a}, R^{6b}, R⁷, R^{8a}, R^{8b}, R^{9a}, R^{9b}, R^{9c}, and R¹⁵ are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁ alkyl, C₁₋₆ alkylthio, C₁₋₆ alkylsulfinyl, C₁ alkylsulfonyl, N—(C₁ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R¹⁰ is selected from the group consisting of C₃₋₁₂ cycloalkyl, C₃₋₁₂ cycloalkenyl, C₅₋₁₂ aryl, 3- to 12-membered heterocycloalkenyl, 5- to 12-membered heteroaryl, and C₂₋₆ alkenyl, wherein R¹⁰ is optionally substituted with one or more substituents selected from the group consisting of R^{13a};
wherein R¹¹, R^{12a}, and R^{12b} are independently selected from the group consisting of hydrido, C₅₋₁₂ aryl, 5- to 12-membered heteroaryl, 4- to 18-membered heteroaralkyl, C₁₋₆ alkyl, C₁₋₆ haloalkyl, C₂₋₆ alkenyl, C₂₋₆ alkynyl, C₁₋₆ hydroxyalkyl, C₁₋₆ aminoalkyl, C₂₋₁₂ alkylaminoalkyl, N-N-di(C₁₋₆alkyl)amino(C₁₋₆alkyl), C₁₋₆ alkoxy, C₂₋₁₂ alkoxyalkyl, 3- to 12-membered heterocycloalkyl, 3- to 12-membered heterocycloalkenyl, C₃₋₁₂ cycloalkyl, C₄₋₁₈ cycloalkylalkyl, C₄₋₁₈ aralkyl, and C₄₋₁₈ aralkylamino, wherein said aryl is optionally substituted with one or more radicals selected from the group consisting of C₁₋₆ alkyl, C₁₋₆ aminoalkyl, C₁₋₆ alkoxy and halo, wherein R¹² and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{14a} and R^{14b} are independently selected from the group consisting of hydrido, C₁₋₆ alkyl, 5- to 12-membered heteroaryl, 3- to 12-membered heterocycloalkenyl, C₁₋₆ haloalkyl, C₄₋₁₈ aralkylamino, 4- to 18-membered heteroaralkyl, C₅₋₁₂ aryl, and C₄₋₁₈ aralkyl, wherein said aryl, heteroaryl, heterocycloalkenyl, and aralkyl moieties are optionally substituted with one or more substituents selected from the group consisting of C₁₋₄ alkyl, C₁₋₆ alkoxy, halo, C₁₋₆ haloalkyl, cyano, C₁₋₆ haloalkoxy, C₂₋₁₀ acyl, carboxyl, hydroxy, C₁₋₆ hydroxyalkoxy, phenoxy, benzyloxy, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

4. A compound according to claim 3:
wherein A is selected from the group consisting of cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein A may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, C(═O)R¹³ and —C(═O)NR^{12a}R^{12b};
wherein L¹ and L² are independently selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, methylene, ethylene, propylene, butylene, pentylene, hexylene, ethenylene, propenylene, butenylene, pentenylene, and ethynylene, propynylene, butynylene, pentynylene, wherein said R⁷, methylene, ethylene, propylene, butylene, pentylene, hexylene, ethenylene, propenylene, butenylene, pentenylene, and ethynylene, propynylene, butynylene, pentynylene moieties may be substituted by one or more substituents independently selected from R¹⁵;
wherein R¹ is selected from the group consisting of hydrido, cyano, methyl, ethyl, propyl, butyl, pentyl, hexyl, ethenyl, propenyl, butenyl, pentenyl, chloro, fluoro, bromo, iodo, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, cyanomethyl, cyanoethyl, cyanopropyl, cyanobutyl, cyanopentyl, cyanohexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{b}, —(CH₂)ₑOR^{8a}, (CH)e NR^{8a}R^{b} nd —(CH₂)ₑS(═O)_{f}R¹¹, or R^{4a} together with R^{4a} and the atoms to which they are attached form a 5- to 8-membered heterocyclic ring moiety having the structure: 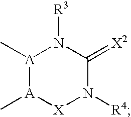
wherein R², R^{2a}, and R³ are independently selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{4a} is selected from the group consisting of hydrido, hydroxyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, or R^{4a} together with R¹ and the atoms to which they are attached form a 5- to 8-membered heterocyclic ring having the structure: 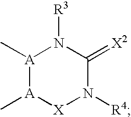
wherein R⁴ is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of methyl, ethyl, propyl, butyl, pentyl, hexyl, cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and phenyl, biphenyl, naphthyl, indenyl, wherein R⁵ may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein R^{6a}, R^{6b}, R⁷, R⁸, R^{8b}, R^{9a}, R^{9b}, R^{9c}, and R¹⁵ are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R¹⁰ is selected from the group consisting of cyanomethyl, cyanoethyl, cyanopropyl, cyanobutyl, cyanopentyl, cyanohexyl, cyclopropenyl, cyclobutenyl, cyclopentenyl, cyclohexenyl, phenyl, biphenyl, naphthyl, indenyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, ethenyl, propenyl, butenyl, and pentenyl, wherein R¹⁰ is optionally substituted with one or more substituents selected from the group consisting of R^{13a};
wherein R¹¹, R^{12a}, and R^{12b} are independently selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, methyl, ethyl, propyl, butyl, pentyl, hexyl, chloromethyl, dichloromethyl, trichloromethyl, fluoromethyl, difluoromethyl, trifluoromethyl, ethenyl, propenyl, butenyl, pentenyl, ethynyl, propynyl, butynyl, pentynyl, hydroxymethyl, hydroxyethyl, hydroxypropyl, hydroxybutyl, hydroxypentyl, hydroxyhexyl, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, N,N-dimethylaminomethyl, N,N-dimethylaminoethyl, N-methyl-N-ethylaminomethyl, N-methyl-N-ethylaminoethyl, N-methyl-N-propylaminomethyl, N-methyl-N-propylaminoethyl, N,N-diethylaminomethyl, N,N-diethylaminoethyl, N-ethyl-N-propylaminomethyl, N-ethyl-N-propylaminoethyl, N,N-dipropylaminomethyl, N,N-dipropylaminoethyl, methoxy, ethoxy, propoxy, butoxy, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl, cyclohexylethyl, benzyl, phenylethyl, benzylamino, and phenylethylamino, wherein said phenyl, biphenyl, naphthyl, indenyl is optionally substituted with one or more radicals selected from the group consisting of methyl, ethyl, propyl, butyl, pentyl, hexyl, aminomethyl, aminoethyl, aminopropyl, aminobutyl, aminopentyl, aminohexyl, methoxy, ethoxy, propoxy, butoxy and chloro, fluoro, bromo, iodo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{14a} and R^{14b} are independently selected from the group consisting of hydrido, methyl, ethyl, propyl, butyl, pentyl, hexyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, chloromethyl, dichloromethyl, trichloromethyl, fluoromethyl, difluoromethyl, trifluoromethyl, benzylamino, phenylethylamino, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, phenyl, biphenyl, naphthyl, indenyl, benzyl, and phenylethyl, wherein said phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, benzyl, and phenylethyl moieties are optionally substituted with one or more substituents selected from the group consisting of methyl, ethyl, propyl, butyl, pentyl, hexyl, methoxy, ethoxy, propoxy, butoxy, chloro, fluoro, bromo, iodo, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, benzyloxy, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

5. A compound of Formula VII: 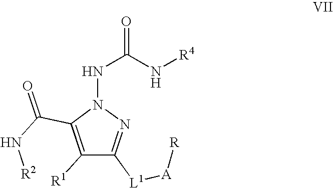
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b} —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}—NR^{13a}C(═O)NR^{14a}R^{14b} —C(═O)R^{13a} and C(═O)NR^{12a}R^{12b};
wherein R is selected from the group consisting of hydrido and -L²R⁵;
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}, —C(═O)R^{7a}—, R^{7a}C(═O)—, —OR^{7a}C(═O)—, and alkynylene, wherein said R^{7a}, alkylene, alkenylene, and alkynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R^{7b}, —C(═O)R^{7b}—, —R^{7b}C(═O)—, —OR^{7b}C(═O)—, alkylene, alkenylene, and alkynylene, wherein said R^{7b}, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR⁸, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, (CH₂)e OR^{8a}, —(CH₂)ₑNR^{8a}R^{8b}, and —(CH₂)ₑS(═O)_{f}R¹¹;
wherein R¹² is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR¹³SO₂NR^{14a}R^{14b}, —NR^{13a}C(═O)NR^{14a}R^{14b}, —C(═O)R^{13a}, and C(═O)NR^{12a}R^{12b};
wherein R^{7a} is selected from the group consisting of hydrido, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{7b}, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, O, N, and NR^{13a};
wherein a, d, and e are independently selected from the group consisting of 0, 1, 2, 3, 4, 5, 6, 7, or 8;
wherein b, c, and f are independently selected from the group consising of 0, 1, or 2;
or a pharmaceutically-acceptable salt thereof.

6. A compound of Formula X: 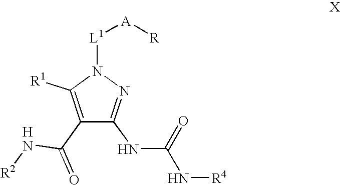
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹¹, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R is selected from the group consisting of hydrido and -L²R⁵;
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}, —C(═O)R^{7a}, R^{7a}C(═O)—, —OR^{7a}C(═O)—, and alkynylene, wherein said R^{7a}, alkylene, alkenylene, and alkynylene moieties may be substituted by one or more substituents independently selected from R⁵;
wherein L² is selected from the group consisting of a bond, —O—, —S(═O)_{c}—, —O(CH₂)_{d}—, —C(═O)—, —OC(═O)—, —C(═O)O—, —OC(═O)R^{7b}—, —C(═O)R^{7b}—, —R^{7b}C(═O)—, —OR^{7b}C(═O)—, alkylene, alkenylene, and alkynylene, wherein said R^{7b}, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR^{8a}, (CH₂)ₑC(═O)R^{8a}, —(CH₂)ₑC(═O)OR^{8a}, (CH₂)ₑC(O)NR⁸R^{b}, —NR^{8a}R^{8b}, (CH₂)ₑOR^{8a}, —(CH₂)ₑNR^{8a}R^{8b}, and —(CH₂)ₑS(═O)_{f}R¹¹;
wherein R² is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂, —NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R^{7a} is selected from the group consisting of hydrido, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{7b}, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, SO₂₁₀, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, O, N, and NR^{13a};
wherein a, d, and e are independently selected from the group consisting of 0, 1, 2, 3, 4, 5, 6, 7, or 8;
wherein b, c, and f are independently selected from the group consising of 0, 1, or 2;
or a pharmaceutically-acceptable salt thereof.

7. A compound according to claim 5 or 6:
wherein A is selected from the group consisting of C₃₋₁₂ cycloalkyl, C₃₋₁₂ cycloalkenyl, 3- to 12-membered heterocycloalkyl, 3- to 12-membered heterocycloalkenyl, C₅₋₁₂ aryl, and 5- to 12-membered heteroaryl, wherein A may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, (CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂, —NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}, —R^{7a}C(═O)—, —OR^{7a}C(═O)—, and C₂₋₆ alkynylene, wherein said R⁷ and alkynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, C₁₋₆ alkylene, C₂₋₆ alkenylene, and C₂₋₆ alkynylene, wherein said R⁷, alkylene, alkenylene, and alkynylene moieties may be substituted by one or more substituents independently selected from R¹⁵;
wherein R¹ is selected from the group consisting of hydrido, cyano, C₁₋₆ alkyl, C₂₋₆ alkenyl, halo, C₁₋₆ haloalkyl, C₁₋₆ hydroxyalkyl, cyano(C₁₋₆ alkyl), C₅₋₁₂ aryl, 5- to 12-membered heteroaryl, —OR^{8a}, —(CH₂)ₑC(═O)R⁸, —(CH₂)ₑC(═O)OR^{8a}, —(CH₂)ₑC(═O)NR^{8a}R^{b}, —NR^{8a}R^{8b}, —(CH₂)ₑOR^{8a}, —(CH₂)ₑNR^{8a}R^{8b}, and —(CH₂)ₑS(═O)_{f}R¹¹;
wherein R² is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R¹⁴ is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R⁵ is selected from the group consisting of C₁₋₆ alkyl, C₃₋₁₂ cycloalkyl, C₃₋₁₂ cycloalkenyl, 3- to 12-membered heterocycloalkyl, 3- to 12-membered heterocycloalkenyl, 5- to 12-membered heteroaryl, and C₅₋₁₂ aryl, wherein R⁵ may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)OR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO NR^{14a}R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{7b}, R^{8a}, R^{8b}, R^{9a}, R^{9b}, R^{9c}, and R¹⁵ are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₂₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R¹⁰ is selected from the group consisting of C₃₋₁₂ cycloalkyl, C₃₋₁₂ cycloalkenyl, C₅₋₁₂ aryl, 3- to 12-membered heterocycloalkenyl, 5- to 12-membered heteroaryl, and C₂₋₆ alkenyl, wherein R¹⁰ is optionally substituted with one or more substituents selected from the group consisting of R^{13a};
wherein R¹¹, R^{12a}, and R^{12b} are independently selected from the group consisting of hydrido, C₅₋₁₂ aryl, 5- to 12-membered heteroaryl, 4- to 18-membered heteroaralkyl, C₁₋₆ alkyl, C₁₋₆ haloalkyl, C₂₋₆ alkenyl, C₂₋₆ alkynyl, C₁₋₆ hydroxyalkyl, C₁₋₆ aminoalkyl, C₂₋₁₂ alkylaminoalkyl, N-N-di(C₁₋₆alkyl)amino(C₁₋₆alkyl), C₁₋₆ alkoxy, C₂₋₁₂ alkoxyalkyl, 3- to 12-membered heterocycloalkyl, 3- to 12-membered heterocycloalkenyl, C₃₋₁₂ cycloalkyl, C₄₋₁₈ cycloalkylalkyl, C₄₋₁₈, aralkyl, and C₄₋₁₈ aralkylamino, wherein said aryl is optionally substituted with one or more radicals selected from the group consisting of C₆ alkyl, C₁₋₆ aminoalkyl, C₁₋₆ alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C-₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{14a} and R^{14b} are independently selected from the group consisting of hydrido, C₁₋₆ alkyl, 5- to 12-membered heteroaryl, 3- to 12-membered heterocycloalkenyl, C₁₋₆ haloalkyl, C₄₋₁₈ aralkylamino, 4- to 18-membered heteroaralkyl, C₅₋₁₂ aryl, and C₄₋₁₈ aralkyl, wherein said aryl, heteroaryl, heterocycloalkenyl, and aralkyl moieties are optionally substituted with one or more substituents selected from the group consisting of C₁₋₆ alkyl, C₁₋₆ alkoxy, halo, C₁₋₆ haloalkyl, cyano, C₁₋₆ haloalkoxy, C₂₋₁₀ acyl, carboxyl, hydroxy, C₁₋₆ hydroxyalkoxy, phenoxy, benzyloxy, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl, wherein R¹⁴⁸ and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

8. A compound according to claim 7:
wherein A is selected from the group consisting of cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein A may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, (CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}, R^{7a}C(═O)—, —OR^{7a}C(═O)—, ethynylene, propynylene, butynylene, and pentynylene, wherein said R⁷, ethynylene, propynylene, butynylene, and pentynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, methylene, ethylene, propylene, butylene, pentylene, hexylene, ethenylene, propenylene, butenylene, pentenylene, ethynylene, propynylene, butynylene, and pentynylene, wherein said R⁷, methylene, ethylene, propylene, butylene, pentylene, hexylene, ethenylene, propenylene, butenylene, pentenylene, ethynylene, propynylene, butynylene, and pentynylene moieties may be substituted by one or more substituents independently selected from R¹⁵;
wherein R¹ is selected from the group consisting of hydrido, cyano, methyl, ethyl, propyl, butyl, pentyl, hexyl, ethenyl, propenyl, butenyl, pentenyl, chloro, fluoro, bromo, iodo, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, cyanomethyl, cyanoethyl, cyanopropyl, cyanobutyl, cyanopentyl, cyanohexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, —OR^{a}, —(CH₂)ₑC(═O)R⁸⁸, (CH₂)ₑC(═O)OR^{8a}, —(CH₂)ₑC(═O)NR^{8a}R^{8b}, —NR^{8a}R^{8b} —(CH₂)e OR⁸⁸ (CH₂)ₑNR^{8a}R^{8b}, —(CH₂)ₑS(═O)_{f}R¹¹;
wherein R² is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonyl propyl, butoxycarbonyl propyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R⁴ is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R⁵ is selected from the group consisting of methyl, ethyl, propyl, butyl, pentyl, hexyl, cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and phenyl, biphenyl, naphthyl, indenyl, wherein R⁵ may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), (CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R^{7b}, R^{8a}, R^{8b}, R^{9a}, R^{9b}, R^{9c}, and R¹⁵ are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R¹⁰ is selected from the group consisting of cyanomethyl, cyanoethyl, cyanopropyl, cyanobutyl, cyanopentyl, cyanohexyl, cyclopropenyl, cyclobutenyl, cyclopentenyl, cyclohexenyl, phenyl, biphenyl, naphthyl, indenyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, ethenyl, propenyl, butenyl, and pentenyl, wherein R¹⁰ is optionally substituted with one or more substituents selected from the group consisting of R^{13a};
wherein R¹¹, R^{12a}, and R^{12b} are independently selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, methyl, ethyl, propyl, butyl, pentyl, hexyl, chloromethyl, dichloromethyl, trichloromethyl, fluoromethyl, difluoromethyl, trifluoromethyl, ethenyl, propenyl, butenyl, pentenyl, ethynyl, propynyl, butynyl, pentynyl, hydroxymethyl, hydroxyethyl, hydroxypropyl, hydroxybutyl, hydroxypentyl, hydroxyhexyl, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, N,N-dimethylaminomethyl, N,N-dimethylaminoethyl, N-methyl-N-ethylaminomethyl, N-methyl-N-ethylaminoethyl, N-methyl-N-propylaminomethyl, N-methyl-N-propylaminoethyl, N,N-diethylaminomethyl, N,N-diethylaminoethyl, N-ethyl-N-propylaminomethyl, N-ethyl-N-propylaminoethyl, N,N-dipropylaminomethyl, N,N-dipropylaminoethyl, methoxy, ethoxy, propoxy, butoxy, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl, cyclohexylethyl, benzyl, phenylethyl, benzylamino, and phenylethylamino, wherein said phenyl, biphenyl, naphthyl, indenyl is optionally substituted with one or more radicals selected from the group consisting of methyl, ethyl, propyl, butyl, pentyl, hexyl, aminomethyl, aminoethyl, aminopropyl, aminobutyl, aminopentyl, aminohexyl, methoxy, ethoxy, propoxy, butoxy and chloro, fluoro, bromo, iodo, wherein R^{12a} and R^{12b} may be taken together to form a 3- to 7-membered carbocyclic ring having from 1 to 3 heteroatoms selected from S, SO, SO₂, O, N, and NR^{13a};
wherein R¹³a and R^{13b} are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{14a} and R^{14b} are independently selected from the group consisting of hydrido, methyl, ethyl, propyl, butyl, pentyl, hexyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, chloromethyl, dichloromethyl, trichloromethyl, fluoromethyl, difluoromethyl, trifluoromethyl, benzylamino, phenylethylamino, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, phenyl, biphenyl, naphthyl, indenyl, benzyl, and phenylethyl, wherein said phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, benzyl, and phenylethyl moieties are optionally substituted with one or more substituents selected from the group consisting of methyl, ethyl, propyl, butyl, pentyl, hexyl, methoxy, ethoxy, propoxy, butoxy, chloro, fluoro, bromo, iodo, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, benzyloxy, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

9. A compound of Formula VIIIl: 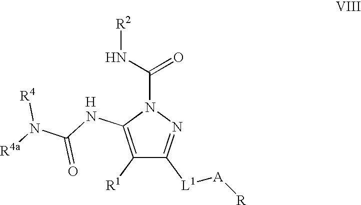
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{a}SO₂NR^{14a}R^{14b}, —NR^{13a}C(═O)NR^{14a}R^{14b}, C(═O)R^{13a}, and —C(═O)NR^{12a}R^{12b};
wherein R is selected from the group consisting of hydrido and -L²R⁵;
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, C(═O)R^{7a}—, R^{7a}C(═O)—, —OR^{7a}C(═O)—, and alkynylene, wherein said R^{7a}, alkylene, alkenylene, and alkynylene moieties may be substituted by one or more substituents independently selected from R¹¹;
wherein L² is selected from the group consisting of a bond, —O—, —S(═O)_{c}—, —O(CH₂)_{d}—, —C(═O)—, —OC(═O)—, —C(═O)O—, —OC(═O)R^{7b}—, C(═O)R^{7b}—, —R^{7b}C(═O), —OR^{7b}C(═O)—, alkylene, alkenylene, and alkynylene, wherein said R^{7b}, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, —(CH₂)ₑOR^{a}, —(CH₂)ₑNR^{8a}R^{8b}, and —(CH₂)ₑS(═O)_{f}R¹¹, or R^{4a} together with R^{14a} and the atoms to which they are attached form a heterocyclic ring moiety having the structure: 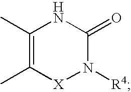
wherein R¹² is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R^{4a} is selected from the group consisting of hydrido, hydroxyl, alkoxy, alkyl, haloalkyl, aryl, and heteroaryl, or R^{4a} together with R¹ and the atoms to which they are attached form a heterocyclic ring having the structure: 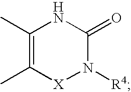
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl, and wherein R⁴, when R¹ together with R^{4a} and the atoms to which they are attached form a heterocyclic ring having 6 or more members, may form a double bond between the nitrogen to which R⁴ is attached and X;
wherein X is selected from the group consisting of a bond, (CHR^{9a})ₓ(CR^{9b})_{y}(CHR^{c})_{z}—, —NR^{9a}—, —CR⁹a═CR^{9b}—(CHR^{9c})_{g}, —(CH₂)ₙO—, —(CH₂)ₕS—, —NR^{9a}—NR^{9b}—, —N═N—, —O—NR^{9a}, —S—NR^{9a}—, —CH═CH—CH₂—, —CH═CH—NR^{9a} —, —CH₂—O—CH₂—, —CH₂—S—CH₂—, —CH₂—N═N—, —NR^{9a}—O—CH₂—, —CH₂—NR^{9a}—S—, and —NR^{9a}—S—CH₂—;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)₀CO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R^{7a} is selected from the group consisting of hydrido, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{7b}, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R¹², and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, O, N, and NR^{13a};
wherein a, d, and e are independently selected from the group consisting of 0, 1, 2, 3, 4, 5, 6, 7, or 8;
wherein b, c, f, and h are independently selected from the group consising of 0, 1, or 2;
wherein g is 0 or 1;
wherein x, y, and z are independently 0, 1, 2, or 3, and wherein x+y+z<4;
or a pharmaceutically-acceptable salt thereof.

10. A compound of Formula IX: 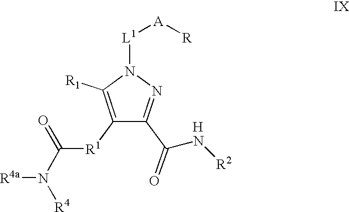
wherein A is selected from the group consisting of cycloalkyl, cycloalkenyl, heterocycloalkyl, heterocycloalkenyl, aryl, and heteroaryl, wherein A may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R is selected from the group consisting of hydrido and -L²R⁵;
wherein L¹ is selected from the group consisting of a bond, —O—, —S(═O)_{c}—, —O(CH₂)_{d}—, —C(═O)—, —OC(═O)—, —C(═O)O—, —OC(═O)R^{a}, —C(═O)R^{7a}, R^{7a}C(═O), —OR^{a}C(═O)—, and alkynylene, wherein said R^{7a}, alkylene, alkenylene, and alkynylene moieties may be substituted by one or more substituents independently selected from R¹⁵;
wherein L² is selected from the group consisting of a bond, —O—, —S(═O)ᵣ—, —O(CH₂)_{d}—, —C(═O)—, —OC(═O)—, —C(═O)O—, —OC(═O)R^{7b}—, —C(═O)R^{7b}, —R^{7b}C(═O)—, —OR^{7b}C(═O)—, alkylene, alkenylene, and alkynylene, wherein said R^{7b}, alkylene, alkenylene, and alkynylene moieties may be substituted by one or more substituents independently selected from R¹⁵;
wherein R¹ is selected from the group consisting of hydrido, cyano, alkyl, alkenyl, halo, haloalkyl, hydroxyalkyl, cyanoalkyl, aryl, heteroaryl, —OR⁸, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, —(CH₂)ₑOR^{8a}, —(CH₂)₆NR^{8a}R^{8b}, and —(CH₂)ₑS(═O)_{f}R¹¹, or R¹ together with R^{4a} and the atoms to which they are attached form a heterocyclic ring moiety having the structure: 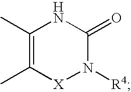
wherein R² is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl;
wherein R^{4a} is selected from the group consisting of hydrido, hydroxyl, alkoxy, alkyl, haloalkyl, aryl, and heteroaryl, or R^{4a} together with R¹ and the atoms to which they are attached form a heterocyclic ring having the structure: 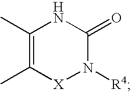
wherein R⁴ is selected from the group consisting of hydrido, hydroxyl, amino, hydroxyalkyl, alkoxyalkyl, aminoalkyl, alkoxycarbonylalkyl, alkylcarbonylalkyl, aminocarbonylalkyl, heterocycloalkyl, alkoxy, alkyl, haloalkyl, aryl, and heteroaryl, and wherein R⁴, when R¹ together with R^{4a} and the atoms to which they are attached form a heterocyclic ring having 6 or more members, may form a double bond between the nitrogen to which R⁴ is attached and X;
wherein X is selected from the group consisting of a bond, —(CHR^{9a})ₓ(CHR^{9b})_{y}(CHR^{9c})_{z}—, —NR^{9a}—, —CR^{9a}═CR^{9b}—(CHR^{9c})_{g}—, —(CH₂)ₕO—, —(CH₂)ₕS—, —NR^{9a}NR^{9b}—, —N═N—, —ONR^{9a}, SNR^{9a}, —CH═CH—CH₂—, —CH═CH—NR^{9a}—, —CH₂—O—CH₂—, —CH₂—S—CH₂—, —CH₂—N═N—, —NR^{9a}—O—CH₂—, —CH₂—NR^{9a}—S—, and —NR^{9a}—S—CH₂—;
wherein R⁵ is selected from the group consisting of alkyl, cycloalkyl, cycloalkenyl, heterocycloalkyl, heterocycloalkenyl, heteroaryl, and aryl, wherein R⁵ may be optionally substituted by one or more substituents selected from the group consisting of halo, alkylsulfinyl, alkylsulfonyl, cyano, alkoxycarbonyl, alkylaryl, alkylheterocycloalkyl, alkylheterocycloalkenyl, alkylheteroaryl, alkyl, alkenyl, alkynyl, haloalkyl, hydroxyalkyl, haloalkoxy, nitro, acylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)S(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and C(═O)NR^{12a}R^{12b};
wherein R^{7a} is selected from the group consisting of hydrido, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{7b}, R^{8a}, R^{8b}, R^{9a}, R^{9b}, R^{9c}, and R¹⁵ are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R¹⁰ is selected from the group consisting of cycloalkyl, cycloalkenyl, aryl, heterocycloalkenyl, heteroaryl, and alkenyl, wherein R¹⁰ is optionally substituted with one or more substituents selected from the group consisting of R^{13a};
wherein R¹¹, R^{12a}, and R^{12b} are independently selected from the group consisting of hydrido, aryl, heteroaryl, heteroaralkyl, alkyl, haloalkyl, alkenyl, alkynyl, hydroxyalkyl, aminoalkyl, alkylaminoalkyl, N,N-dialkylaminoalkyl, alkoxy, alkoxyalkyl, heterocycloalkyl, heterocycloalkenyl, cycloalkyl, cycloalkylalkyl, aralkyl, and aralkylamino, wherein said aryl is optionally substituted with one or more radicals selected from the group consisting of alkyl, aminoalkyl, alkoxy and halo, wherein R^{12a} and R^{12b} may be taken together to form a 3- to 7-membered carbocyclic ring having from 1 to 3 heteroatoms selected from S, SO, S O, N, and NR^{13a};
wherein R^{13a} and R^{13b} are independently selected from the group consisting of hydrido, alkyl, aryl, heteroaryl, aralkyl, heterocycloalkenyl, cycloalkyl, heterocycloalkyl, haloalkyl, aralkylamino, amino, aminoalkyl, aminoacyl, nitro, azido, and heteroaralkyl, wherein said alkyl, aryl, heteroaryl, aminoalkyl, and aralkyl moieties are optionally substituted with one or more substituents selected from the group consisting of alkylsulfonamido, sulfamyl, alkyl, alkylthio, alkylsulfinyl, alkylsulfonyl, N-alkylamino, aminoalkyl, alkylaminoalkyl, alkoxy, halo, acyloxy, oxy, formyl, haloalkyl, cyano, haloalkoxy, acyl, carboxyl, hydroxy, hydroxyalkoxy, phenoxy, nitro, azido, benzyloxy, N,N-dialkylaminoacyl, thioalkyl, aminoacyloxy, thiocyanato, isothiocyanato, alkyldioxy, hydroxyalkyl, N-alkylamino, alkoxycarbonyl, alkoxyalkyl, alkenylamino, alkynylamino, alkenyl, alkynyl, N,N-dialkylaminoalkoxy, heterocycloalkyl, heterocycloalkenyl, and heteroaryl;
wherein R^{14a} and R^{14b} are independently selected from the group consisting of hydrido, alkyl, heteroaryl, heterocycloalkenyl, haloalkyl, aralkylamino, heteroaralkyl, aryl, and aralkyl, wherein said aryl, heteroaryl, heterocycloalkenyl, and aralkyl moieties are optionally substituted with one or more substituents selected from the group consisting of alkyl, alkoxy, halo, haloalkyl, cyano, haloalkoxy, acyl, carboxyl, hydroxy, hydroxyalkoxy, phenoxy, benzyloxy, N,N-dialkylaminoalkoxy, heterocycloalkyl, heterocycloalkenyl, and heteroaryl, wherein R^{14a} and R^{14b} may be taken together to form a 3- to 7-membered carbocyclic ring having 1 to 3 heteroatoms selected from S, SO, SO₂, O, N, and NR^{13a};
wherein a, d, and e are independently selected from the group consisting of 0, 1, 2, 3, 4, 5, 6, 7, or 8;
wherein b, c, f, and h are independently selected from the group consising of 0, 1, or 2;
wherein g is 0 or 1;
wherein x, y, and z are independently 0, 1, 2, or 3, and wherein x+y+z<4;
or a pharmaceutically-acceptable salt thereof.

11. A compound according to claim 9 or 10:
wherein A is selected from the group consisting of C₃₋₁₂ cycloalkyl, C₃₋₁₂ cycloalkenyl, 3- to 12-membered heterocycloalkyl, 3- to 12-membered heterocycloalkenyl, C₅₋₁₂ aryl, and 5- to 12-membered heteroaryl, wherein A may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a} (═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, C(═O)R^{7a}—, R^{7a}C(═O), —OR^{7a}C(═O)—, and C₂₋₆ alkynylene, wherein said R⁷ and alkynylene moieties may be substituted by one or more substituents independently selected from R⁵;
wherein L² is selected from the group consisting of a bond, —O—, —S(═O)_{c}, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, C₁₋₆ alkylene, C₂₋₆ alkenylene, and C₂₋₆ alkynylene, wherein said R⁷, alkylene, alkenylene, and alkynylene moieties may be substituted by one or more substituents independently selected from R¹⁵;
wherein R¹ is selected from the group consisting of hydrido, cyano, C₁₋₆ alkyl, C₂₋₆ alkenyl, halo, C₁₋₄ haloalkyl, C₁₋₆ hydroxyalkyl, cyano(C₁₋₆ alkyl), C₅₋₁₂ aryl, 5- to 12-membered heteroaryl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, —(CH₂)ₑOR^{8a}, —(CH₂)ₑNR^{8a}R^{8b}, and —(CH₂)ₑS(═O)_{f}R¹¹, or R¹ together with R^{4a} and the atoms to which they are attached form a 5- to 8-membered heterocyclic ring moiety having the structure: 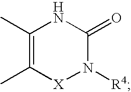
wherein R² is selected from the group consisting of hydrido, hydroxyl, amino, C₁₋₆ hydroxyalkyl, C₂₋₁₂ alkoxyalkyl, C₁₋₆ aminoalkyl, C₃₋₁₃ alkoxycarbonylalkyl, C₃₋₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl;
wherein R^{4a} is selected from the group consisting of hydrido, hydroxyl, C₁₋₆ alkoxy, C₁₋₆ alkyl, C₁₋₆ haloalkyl, C₅₋₁₂ aryl, and 5- to 12-membered heteroaryl, or R^{4a} together with R¹ and the atoms to which they are attached form a 5- to 8-membered heterocyclic ring having the structure: 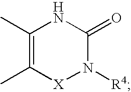
wherein R⁴ is selected from the group consisting of hydrido, hydroxyl, amino, C₁₋₆ hydroxyalkyl, C₂₋₁₂ alkoxyalkyl, C₁₋₆ aminoalkyl, C₃₋₃ alkoxycarbonylalkyl, C₃₋₁₃ alkylcarbonylalkyl, C₂₋₇ aminocarbonylalkyl, 3- to 12-membered heterocycloalkyl, C₁₋₆ alkoxy, C₁₋₆ alkyl, C₁₋₆ haloalkyl, C₅₋₁₂ aryl, and 5- to 12-membered heteroaryl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of C₁₋₄ alkyl, C₃₋₁₂ cycloalkyl, C₃₋₁₂ cycloalkenyl, 3- to 12-membered heterocycloalkyl, 3- to 12-membered heterocycloalkenyl, 5- to 12-membered heteroaryl, and C₅₋₁₂ aryl, wherein R⁵ may be optionally substituted by one or more substituents selected from the group consisting of halo, C₁₋₆ alkylsulfinyl, C₁₋₆ alkylsulfonyl, cyano, C₂₋₇ alkoxycarbonyl, C₄₋₁₈ alkylaryl, (C₁₋₆ alkyl)(3- to 12-membered heterocycloalkyl), (C₁₋₆ alkyl)(3- to 12-membered heterocycloalkenyl), (C₁₋₆ alkyl)(5- to 12-membered heteroaryl), C₁₋₆ alkyl, C₂₋₆ alkenyl, C₂₋₆ alkynyl, C₁₋₆ haloalkyl, C₁₋₆ hydroxyalkyl, C₁₋₆ haloalkoxy, nitro, C₂₋₁₀ acylamino, R¹⁰, —OR¹¹, C(═N)NR^{12a}R^{12b}, —NR^{12a}—N(═CR^{13a}R^{13b}), —(CH₂)ₐOR¹¹, (CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₂₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{7b}, R^{7a}, R^{8b}, R^{9a}, R^{9b}, R^{9c}, and R¹⁵ are independently selected from the group consisting of hydrido, C₁₋₆ alkyl, C₅₋₁₂ aryl, 5- to 12-membered heteroaryl, C₄₋₁₈ aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁₋₆ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₆ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₇ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R¹⁰ is selected from the group consisting of C₃₋₁₂ cycloalkyl, C₃₋₁₂ cycloalkenyl, C₅₋₁₂ aryl, 3- to 12-membered heterocycloalkenyl, 5- to 12-membered heteroaryl, and C₂₋₆ alkenyl, wherein R¹⁰ is optionally substituted with one or more substituents selected from the group consisting of R^{13a};
wherein R¹, R^{12a}, and R^{12b} are independently selected from the group consisting of hydrido, C₅₋₁₂ aryl, 5- to 12-membered heteroaryl, 4- to 18-membered heteroaralkyl, C₁₋₆ alkyl, C₁₋₆ haloalkyl, C₂₋₆ alkenyl, C₂₋₆ alkynyl, C₁₋₆ hydroxyalkyl, C₁₋₆ aminoalkyl, C₂₋₁₂ alkylaminoalkyl, N-N-di(C₁₋₆alkyl)amino(C₁₋₁₆alkyl), C₁₋₆ alkoxy, C₂₋₁₂ alkoxyalkyl, 3- to 12-membered heterocycloalkyl, 3- to 12-membered heterocycloalkenyl, C₃₋₁₂ cycloalkyl, C₄₋₁₈ cycloalkylalkyl, C₄₋₁₈ aralkyl, and C₄₋₁₈ aralkylamino, wherein said aryl is optionally substituted with one or more radicals selected from the group consisting of C₁₋₆ alkyl, C₁₋₆ aminoalkyl, C₁₋₆ alkoxy and halo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, C₁₋₆ alkyl, C₁₋₂ aryl, 5- to 12-membered heteroaryl, C₄₋₁₈, aralkyl, 3- to 12-membered heterocycloalkenyl, C₃₋₁₂ cycloalkyl, 3- to 12-membered heterocycloalkyl, C₁₋₆ haloalkyl, C₄₋₁₈ aralkylamino, amino, C₁₋₆ aminoalkyl, C₂₋₁₀ aminoacyl, nitro, azido, and 4- to 18-membered heteroaralkyl, wherein said alkyl, aryl, heteroaryl, aminoalkyl, and aralkyl moieties are optionally substituted with one or more substituents selected from the group consisting of C₁₋₆ alkylsulfonamido, sulfamyl, C₁₋₆ alkyl, C₁₋₆ alkylthio, C₁₋₆ alkylsulfinyl, C₁₋₆ alkylsulfonyl, N-(C₁₋₆ alkyl)amino, C₁₋₆ aminoalkyl, C₂₋₁₂ alkylaminoalkyl, C₁ alkoxy, halo, C₂₋₁₀ acyloxy, oxy, formyl, C₁₋₆ haloalkyl, cyano, C₁₋₆ haloalkoxy, C₂₋₁₀ acyl, carboxyl, hydroxy, C₁₋₆ hydroxyalkoxy, phenoxy, nitro, azido, benzyloxy, N,N-di(C₁₋₁ alkyl)amino(C₂₋₁₀ acyl), C₁₋₆ thioalkyl, C₂₋₁₀ aminoacyloxy, thiocyanato, isothiocyanato, C₁₋₆ alkyldioxy, C₁₋₆ hydroxyalkyl, N-(C₁₋₆ alkyl)amino, C₂₋₄ alkoxycarbonyl, C₂₋₁₂ alkoxyalkyl, C₂₋₆ alkenylamino, C₂₋₆ alkynylamino, C₂₋₆ alkenyl, C₂₋₆ alkynyl, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl;
wherein R^{14a} and R^{14b} are independently selected from the group consisting of hydrido, C₁₋₆ alkyl, 5- to 12-membered heteroaryl, 3- to 12-membered heterocycloalkenyl, C₁₋₆ haloalkyl, C₄₋₁₈ aralkylamino, 4- to 18-membered heteroaralkyl, C₅₋₁₂ aryl, and C₄₋₁₈ aralkyl, wherein said aryl, heteroaryl, heterocycloalkenyl, and aralkyl moieties are optionally substituted with one or more substituents selected from the group consisting of C₁₋₆ alkyl, C₁₋₆ alkoxy, halo, C₁₋₆ haloalkyl, cyano, C₁₋₆ haloalkoxy, C₂₋₁₀ acyl, carboxyl, hydroxy, C₁₋₆ hydroxyalkoxy, phenoxy, benzyloxy, N,N-di(C₁₋₆ alkyl)amino(C₁₋₆ alkoxy), 3- to 12-membered heterocycloalkyl, 3- to 12-membered heterocycloalkenyl, and 5- to 12-membered heteroaryl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

12. A compound according to claim 11:
wherein A is selected from the group consisting of cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein A may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, —C(═N)NR^{12a}R^{12b}, —NR^{12a}—N(═CR^{13a}R^{13b}), —(CH₂)ₐOR¹¹, —(CH₂)ₐNR^{12a}R^{12b}, —(CH₂)ₐS(═O)_{b}R¹¹, (CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein L¹ is selected from the group consisting of a bond, —O—, —S(═O)_{c}—, —O(CH₂)_{d}—, —C(═O)—, —OC(═O)—, —C(═O)O—, —OC(═O)R^{7a}—, —C(═O)R^{7a}—, R^{7a}C(═O)—, —OR^{7a}C(═O)—, ethynylene, propynylene, butynylene, and pentynylene, wherein said R⁷, ethynylene, propynylene, butynylene, and pentynylene moieties may be substituted by one or more substituents independently selected from R¹⁵;
wherein L² is selected from the group consisting of a bond, —O—, —S(═O)_{c}—, —O(CH₂)_{d}—, —C(═O)—, —OC(═O)—, —C(═O)O—, —OC(═O)R⁷—, —C(═O)R⁷—, —R⁷C(═O)—, —OR⁷C(═O)—, methylene, ethylene, propylene, butylene, pentylene, hexylene, ethenylene, propenylene, butenylene, pentenylene, ethynylene, propynylene, butynylene, and pentynylene, wherein said R⁷, methylene, ethylene, propylene, butylene, pentylene, hexylene, ethenylene, propenylene, butenylene, pentenylene, ethynylene, propynylene, butynylene, and pentynylene moieties may be substituted by one or more substituents independently selected from R⁵;
wherein R¹ is selected from the group consisting of hydrido, cyano, methyl, ethyl, propyl, butyl, pentyl, hexyl, ethenyl, propenyl, butenyl, pentenyl, chloro, fluoro, bromo, iodo, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, cyanomethyl, cyanoethyl, cyanopropyl, cyanobutyl, cyanopentyl, cyanohexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, —OR^{8a}, —(CH₂)ₑC(═O)R^{8a}, —(CH₂)ₑC(═O)OR^{8a}, —(CH₂)ₑC(═O)NR^{8a}R^{8b}, —NR^{8a}R^{8b}, O(CH₂)ₑR^{8a}, (CH₂)ₑNR^{8a}R^{b}, and —(CH₂)ₑS(═O)_{f}R¹¹, or R¹ together with R^{14a} and the atoms to which they are attached form a 5- to 8-membered heterocyclic ring moiety having the structure: 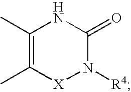
wherein R² is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{4a} is selected from the group consisting of hydrido, hydroxyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, or R^{4a} together with R¹ and the atoms to which they are attached form a 5- to 8-membered heterocyclic ring having the structure: 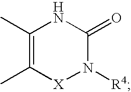
wherein R⁴ is selected from the group consisting of hydrido, hydroxyl, amino, hydroxymethyl, hydroxyethyl, hydroxypropyl, hydroxybutyl, hydroxypentyl, hydroxyhexyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, aminomethyl, aminoethyl, aminopropyl, aminobutyl, aminopentyl, aminohexyl, methoxycarbonylmethyl, ethoxycarbonylmethyl, propoxycarbonylmethyl, butoxycarbonylmethyl, methoxycarbonylethyl, ethoxycarbonylethyl, propoxycarbonylethyl, butoxycarbonylethyl, methoxycarbonylpropyl, ethoxycarbonylpropyl, propoxycarbonylpropyl, butoxycarbonylpropyl, methoxycarbonylbutyl, ethoxycarbonylbutyl, propoxycarbonylbutyl, butoxycarbonylbutyl, methylcarbonylmethyl, ethylcarbonylmethyl, propylcarbonylmethyl, butylcarbonylmethyl, pentylcarbonylmethyl, hexylcarbonylmethyl, methylcarbonylethyl, ethylcarbonylethyl, propylcarbonylethyl, butylcarbonylethyl, pentylcarbonylethyl, hexylcarbonylethyl, methylcarbonylpropyl, ethylcarbonylpropyl, propylcarbonylpropyl, butylcarbonylpropyl, pentylcarbonylpropyl, hexylcarbonylpropyl, methylcarbonylbutyl, ethylcarbonylbutyl, propylcarbonylbutyl, butylcarbonylbutyl, pentylcarbonylbutyl, hexylcarbonylbutyl, aminocarbonylmethyl, aminocarbonylethyl, aminocarbonylpropyl, aminocarbonylbutyl, aminocarbonylpentyl, aminocarbonylhexyl, piperidinyl, pyrrolidinyl, pyrazolidinyl, imidazolidinyl, isoxazolidinyl, oxazolidinyl, methoxy, ethoxy, propoxy, butoxy, methyl, ethyl, propyl, butyl, pentyl, hexyl, chloromethyl, dichloromethyl, trichloromethyl, fluoromethyl, difluoromethyl, trifluoromethyl, phenyl, biphenyl, naphthyl, indenyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and wherein R⁴, when R¹ together with R^{4a} and the atoms to which they are attached form a heterocyclic ring having 6 or more members, may form a double bond between the nitrogen to which R⁴ is attached and X;
wherein R⁵ is selected from the group consisting of methyl, ethyl, propyl, butyl, pentyl, hexyl, cyclopropyl, cyclobutyl, cyclopentyl, cyclohexyl, cyclopropenyl, cyclobutenyl, cyclopentenyl, cyclohexenyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, and phenyl, biphenyl, naphthyl, indenyl, wherein R⁵ may be optionally substituted by one or more substituents selected from the group consisting of chloro, fluoro, bromo, iodo, methylsulfinyl, ethylsulfinyl, propylsulfinyl, butylsulfinyl, methylsulfonyl, ethylsulfonyl, propylsulfonyl, butylsulfonyl, cyano, methoxycarbonyl, ethoxycarbonyl, propoxycarbonyl, butoxycarbonyl, methylphenyl, ethylphenyl, propylphenyl, butylphenyl, pentylphenyl, hexylphenyl, methylnaphthyl, ethylnaphthyl, propylnaphthyl, butylnaphthyl, pentylnaphthyl, hexylnaphthyl, methylpiperidinyl, methylpyrrolidinyl, methylpyrazolidinyl, methylimidazolidinylmethyl, methylisoxazolidinyl, methyloxazolidinyl, ethylpiperidinyl, ethylpyrrolidinyl, ethylpyrazolidinyl, ethylimidazolidinyl, ethylisoxazolidinyl, ethyloxazolidinyl, propylpiperidinyl, propylpyrrolidinyl, propylpyrazolidinyl, propylimidazolidinyl, propylisoxazolidinyl, propyloxazolidinyl, methylisoindolyl, methyldihydroindolyl, methylisoindoline, methyldihydrothiophenyl, methyldihydropyrrolyl, methyldihydrofuryl, methyldihydropyrazolyl, methyldihydroimidazolyl, methyldihydroisoxazolyl, methyldihydrooxazolyl, ethylisoindolyl, ethyldihydroindolyl, ethylisoindoline, ethyldihydrothiophenyl, ethyldihydropyrrolyl, ethyldihydrofuryl, ethyldihydropyrazolyl, ethyldihydroimidazolyl, ethyldihydroisoxazolyl, ethyldihydrooxazolyl, propylisoindolyl, propyldihydroindolyl, propylisoindoline, propyldihydrothiophenyl, propyldihydropyrrolyl, propyldihydrofuryl, propyldihydropyrazolyl, propyldihydroimidazolyl, propyldihydroisoxazolyl, propyldihydrooxazolyl, methylpyridinyl, methylbenzothiophenyl, methylindolyl, methylisoquinolinyl, methylquinolinyl, methylthienyl, methylpyrrolyl, methylfuryl, methylpyrazolyl, imidazolylmethyl, methylisoxazolyl, methyloxazolyl, methylisoindoledionyl, ethylpyridinyl, ethylbenzothiophenyl, ethylindolyl, ethylisoquinolinyl, ethylquinolinyl, ethylthienyl, ethylpyrrolyl, ethylfuryl, ethylpyrazolyl, ethylimidazolyl, ethylisoxazolyl, ethyloxazolyl, ethylisoindoledionyl, propylpyridinyl, propylbenzothiophenyl, propylindolyl, propylisoquinolinyl, propylquinolinyl, propylthienyl, propylpyrrolyl, propylfuryl, propylpyrazolyl, propylimidazolyl, propylisoxazolyl, propyloxazolyl, propylisoindoledionyl, methyl, ethyl, propyl, butyl, pentyl, hexyl, ethenyl, propenyl, butenyl, pentenyl, ethynyl, propynyl, butynyl, pentynyl, chloromethyl, dichloromethyl, trichloromethyl, fluoromethyl, difluoromethyl, trifluoromethyl, hydroxymethyl, hydroxyethyl, hydroxypropyl, hydroxybutyl, hydroxypentyl, hydroxyhexyl, chloromethoxy, dichloromethoxy, trichloromethoxy, fluoromethoxy, difluoromethoxy, trifluoromethoxy, nitro, methylcarbonylamino, ethylcarbonylamino, propylcarbonylamino, butylcarbonylamino, pentylcarbonylamino, hexylcarbonylamino, phenylcarbonylamino, benzylcarbonylamino, R¹⁰, —OR¹¹, C(═N)NR^{12a}R^{12b}, —NR^{12a}—N(═CR^{13a}R^{13b}), (CH₂)ₐOR¹¹, —(CH₂)ₐNR^{12a}R^{12b}, —(CH₂)ₐS(═O)_{b}R¹¹, —(CH₂)ₐCO₂R¹¹, —SO₂NR^{12a}R^{12b}, —NR^{13a}R^{13b}, —NR^{12a}C(═O)R^{13a}, —NR^{12a}C(═O)OR^{13a}, —NR^{13a}SO₂R^{14a}, —NR^{13a}SO₂NR^{14a}R^{14b}, —NR^{13a}C(═O)NR^{14a}R^{14b}, —C(═O)R^{13a}, and —C(═O)NR^{12a}R^{12b};
wherein R^{7a} is selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R^{7b}, R^{8a}, R^{8b}, R^{9a}, R^{9b}, R^{9c}, and R¹⁵ are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, benzyl, and phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl;
wherein R¹⁰ is selected from the group consisting of cyanomethyl, cyanoethyl, cyanopropyl, cyanobutyl, cyanopentyl, cyanohexyl, cyclopropenyl, cyclobutenyl, cyclopentenyl, cyclohexenyl, phenyl, biphenyl, naphthyl, indenyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, ethenyl, propenyl, butenyl, and pentenyl, wherein R¹⁰ is optionally substituted with one or more substituents selected from the group consisting of R^{13a};
wherein R¹¹, R^{12a}, and R^{12b} are independently selected from the group consisting of hydrido, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, methyl, ethyl, propyl, butyl, pentyl, hexyl, chloromethyl, dichloromethyl, trichloromethyl, fluoromethyl, difluoromethyl, trifluoromethyl, ethenyl, propenyl, butenyl, pentenyl, ethynyl, propynyl, butynyl, pentynyl, hydroxymethyl, hydroxyethyl, hydroxypropyl, hydroxybutyl, hydroxypentyl, hydroxyhexyl, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, N,N-dimethylaminomethyl, N,N-dimethylaminoethyl, N-methyl-N-ethylaminomethyl, N-methyl-N-ethylaminoethyl, N-methyl-N-propylaminomethyl, N-methyl-N-propylaminoethyl, N,N-diethylaminomethyl, N,N-diethylaminoethyl, N-ethyl-N-propylaminomethyl, N-ethyl-N-propylaminoethyl, N,N-dipropylaminomethyl, N,N-dipropylaminoethyl, methoxy, ethoxy, propoxy, butoxy, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, cyclopropylmethyl, cyclopropylethyl, cyclobutylmethyl, cyclobutylethyl, cyclopentylmethyl, cyclopentylethyl, cyclohexylmethyl, cyclohexylethyl, benzyl, phenylethyl, benzylamino, and phenylethylamino, wherein said phenyl, biphenyl, naphthyl, indenyl is optionally substituted with one or more radicals selected from the group consisting of methyl, ethyl, propyl, butyl, pentyl, hexyl, aminomethyl, aminoethyl, aminopropyl, aminobutyl, aminopentyl, aminohexyl, methoxy, ethoxy, propoxy, butoxy and chloro, fluoro, bromo, iodo, wherein R^{12a} and R^{12b} may be taken together to form a 3- to 7-membered carbocyclic ring having from 1 to 3 heteroatoms selected from S, SO, SO₂, O, N, and NR^{13a};
wherein R^{13a} and R^{13b} are independently selected from the group consisting of hydrido, methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, benzyl, phenylethyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, cyclopropyl, cyclobutyl, cyclopentyl, cyclohexyl, piperidinyl, pyrrolidinyl, pyrazolidinyl, imidazolidinyl, isoxazolidinyl, oxazolidinyl, chloromethyl, dichloromethyl, trichloromethyl, fluoromethyl, difluoromethyl, trifluoromethyl, benzylamino, phenylethylamino, amino, aminomethyl, aminoethyl, aminopropyl, aminobutyl, aminopentyl, aminohexyl, aminomethylcarbonyl, aminoethylcarbonyl, aminopropylcarbonyl, aminobutylcarbonyl, aminopentylcarbonyl, aminohexylcarbonyl, aminophenylcarbonyl, aminobenzylcarbonyl, nitro, azido, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, and isoindoledionylethyl, wherein said methyl, ethyl, propyl, butyl, pentyl, hexyl, phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, aminomethyl, aminoethyl, aminopropyl, aminobutyl, aminopentyl, aminohexyl, and benzyl, phenylethyl moieties are optionally substituted with one or more substituents selected from the group consisting of methylsulfonamido, ethylsulfonamido, propylsulfonamido, butylsulfonamido, sulfamyl, methyl, ethyl, propyl, butyl, pentyl, hexyl, methylthio, ethylthio, propylthio, butylthio, methylsulfinyl, ethylsulfinyl, propylsulfinyl, butylsulfinyl, methylsulfonyl, ethylsulfonyl, propylsulfonyl, butylsulfonyl, N-methylamino, N-ethylamino, N-propylamino, aminomethyl, aminoethyl, aminopropyl, aminobutyl, aminopentyl, aminohexyl, methylaminomethyl, ethylaminomethyl, propylaminomethyl, methylaminoethyl, ethylaminoethyl, propylaminoethyl, methylaminopropyl, ethylaminopropyl, propylaminopropyl, methylaminobutyl, ethylaminobutyl, propylaminobutyl, methylaminopentyl, ethylaminopentyl, propylaminopentyl, methylaminohexyl, ethylaminohexyl, propylaminohexyl, methoxy, ethoxy, propoxy, butoxy, chloro, fluoro, bromo, iodo, methylcarbonyloxy, ethylcarbonyloxy, propylcarbonyloxy, butylcarbonyloxy, pentylcarbonyloxy, hexylcarbonyloxy, phenylcarbonyloxy, benzylcarbonyloxy, oxy, formyl, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, nitro, azido, benzyloxy, N,N-dimethylaminomethylcarbonyl, N,N-dimethylaminoethylcarbonyl, N,N-dimethylaminophenylcarbonyl, N-methyl-N-ethylaminomethylcarbonyl, N-methyl-N-ethylaminoethylcarbonyl, N-methyl-N-ethylaminophenylcarbonyl, N-methyl-N-propylaminomethylcarbonyl, N-methyl-N-propylaminoethylcarbonyl, N-methyl-N-propylaminophenylcarbonyl, N,N-diethylaminomethylcarbonyl, N,N-diethylaminoethylcarbonyl, N,N-diethylaminophenylcarbonyl, N-ethyl-N-propylaminomethylcarbonyl, N-ethyl-N-propylaminoethylcarbonyl, N-ethyl-N-propylaminophenylcarbonyl, N,N-dipropylaminomethylcarbonyl, N,N-dipropylaminoethylcarbonyl, N,N-dipropylaminophenylcarbonyl, thiomethyl, thioethyl, thiopropyl, thiobutyl, thiopentyl, thiohexyl, aminomethylcarbonyloxy, aminoethylcarbonyloxy, aminopropylcarbonyloxy, aminobutylcarbonyloxy, aminopentylcarbonyloxy, aminohexylcarbonyloxy, aminophenylcarbonyloxy, aminobenzylcarbonyloxy, thiocyanato, isothiocyanato, methyldioxy, ethyldioxy, propyldioxy, butyldioxy, pentyldioxy, hexyldioxy, hydroxymethyl, hydroxyethyl, hydroxypropyl, hydroxybutyl, hydroxypentyl, hydroxyhexyl, N-methylamino, N-ethylamino, N-propylamino, methoxycarbonyl, ethoxycarbonyl, propoxycarbonyl, butoxycarbonyl, methoxymethyl, methoxyethyl, methoxypropyl, ethoxymethyl, ethoxyethyl, ethoxypropyl, propoxymethyl, propoxyethyl, propoxypropyl, butoxymethyl, butoxyethyl, butoxypropyl, ethenylamino, propenylamino, butenylamino, pentenylamino, ethynylamino, propynylamino, butynylamino, pentynylamino, ethenyl, propenyl, butenyl, pentenyl, ethynyl, propynyl, butynyl, pentynyl, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, and pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl;
wherein R^{14a} and R^{14b} are independently selected from the group consisting of hydrido, methyl, ethyl, propyl, butyl, pentyl, hexyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, chloromethyl, dichloromethyl, trichloromethyl, fluoromethyl, difluoromethyl, trifluoromethyl, benzylamino, phenylethylamino, pyridinylmethyl, pyridinylethyl, benzothiophenylmethyl, benzothiophenylethyl, indolylmethyl, indolylethyl, isoquinolinylmethyl, isoquinolinylethyl, quinolinylmethyl, quinolinylethyl, thienylmethyl, thienylethyl, pyrrolylmethyl, pyrrolylethyl, furylmethyl, furylethyl, pyrazolylmethyl, pyrazolylethyl, imidazolylmethyl, imidazolylethyl, isoxazolylmethyl, isoxazolylethyl, oxazolylmethyl, oxazolylethyl, isoindoledionylmethyl, isoindoledionylethyl, phenyl, biphenyl, naphthyl, indenyl, benzyl, and phenylethyl, wherein said phenyl, biphenyl, naphthyl, indenyl, pyridinyl, benzothiophenyl, indolyl, isoquinolinyl, quinolinyl, thienyl, pyrrolyl, furyl, pyrazolyl, imidazolyl, isoxazolyl, oxazolyl, isoindoledionyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, benzyl, and phenylethyl moieties are optionally substituted with one or more substituents selected from the group consisting of methyl, ethyl, propyl, butyl, pentyl, hexyl, methoxy, ethoxy, propoxy, butoxy, chloro, fluoro, bromo, iodo, chloromethyl, dichloromethyl, trichloromethyl, fluoromethyl, difluoromethyl, trifluoromethyl, cyano, chloromethoxy, dichloromethoxy, trichloromethoxy, fluoromethoxy, difluoromethoxy, trifluoromethoxy, methylcarbonyl, ethylcarbonyl, propylcarbonyl, butylcarbonyl, pentylcarbonyl, hexylcarbonyl, phenylcarbonyl, benzylcarbonyl, carboxyl, hydroxy, hydroxymethoxy, hydroxyethoxy, hydroxypropoxy, hydroxybutoxy, phenoxy, benzyloxy, N,N-dimethylaminomethoxy, N,N-dimethylaminoethoxy, N-methyl-N-ethylaminomethoxy, N-methyl-N-ethylaminoethoxy, N-methyl-N-propylaminomethoxy, N-methyl-N-propylaminoethoxy, N,N-diethylaminomethoxy, N,N-diethylaminoethoxy, N-ethyl-N-propylaminomethoxy, N-ethyl-N-propylaminoethoxy, N,N-dipropylaminomethoxy, N,N-dipropylaminoethoxy, piperidinyl, pyrrolidinyl, pyrazolidinyl, imidazolidinyl, isoxazolidinyl, oxazolidinyl, isoindolyl, dihydroindolyl, isoindoline, dihydrothiophenyl, dihydropyrrolyl, dihydrofuryl, dihydropyrazolyl, dihydroimidazolyl, dihydroisoxazolyl, dihydrooxazolyl, pyridinyl, benzothiophenyl, indolyl, isoquinolinyl, quinolinyl, thienyl, pyrrolyl, furyl, pyrazolyl, imidazolyl, isoxazolyl, oxazolyl, and isoindoledionyl, wherein R^{14a} and R^{14b} may be taken together to form a 3- to 7-membered carbocyclic ring having 1 to 3 heteroatoms selected from S, SO, SO₂, O, N, and NR^{13a};
or a pharmaceutically-acceptable salt thereof.

13. A pharmaceutical composition comprising a compound according to claim 1 or a pharmaceutically-acceptable salt thereof, and a pharmaceutically acceptable carrier, diluent, or adjuvant.

14. A method of treating cancer, inflammation, or an inflammation-associated disorder in a subject, said method comprising administering to the subject having or susceptible to such cancer, inflammation, or an inflammation-associated disorder, a therapeutically-effective amount of a compound according to claim 1.

15. A method according to claim 14 wherein said method is a method of treating arthritis, cancer, asthma, COPD, frailty, diabetes, atherosclerosis, pain, and/or dermatological disease.
